(19) United States
(12) Patent Application Publication
(10) Pub. No.: US 2026/0022386 A1
(43) Pub. Date: Jan. 22, 2026

(54) Single Construct Platform for Simultaneous Delivery of Gene Editing Machinery and Nucleic Acid Cargo

(71) Applicant: Basecamp Research Ltd, London (GB)

(72) Inventors: Jonathan Douglas Finn, Watertown, MA (US); Rahul Kakkar, Sudbury, MA (US); Brett Joseph Gordon Estes, Arlington, MA (US); Yijun Zhang, Braintree, MA (US)

(21) Appl. No.: 18/705,515

(22) PCT Filed: Nov. 1, 2022
(86) PCT No.: PCT/US2022/079035

§ 371 Date: Apr. 26, 2024

Related U.S. Application Data
(60) Provisional application No. 63/274,483, filed on Nov. 1, 2021.
(60) Provisional application No. 63/282,055, filed on Nov. 22, 2021.
(60) Provisional application No. 63/298,941, filed on Jan. 12, 2022.
(60) Provisional application No. 63/318,344, filed on Mar. 9, 2022.
(60) Provisional application No. 63/352,897, filed on Jun. 16, 2022.

(51) Int. Cl. C12N 15/52; A61K 48/00; C12N 9/12; C12N 9/16; C12N 15/113; C12N 15/62; C12N 15/85

(52) U.S. Cl. CPC C12N 15/52; A61K 48/005; C12N 9/1276; C12N 9/16; C12N 15/113; C12N 15/62; C12N 15/85; C12Y 207/07049; C12N 2310/532

(57) ABSTRACT

The present disclosure provides nucleic acid compositions, methods, and an overall platform for site-specific genetic engineering using Programmable Addition via Site-Specific Targeting Elements (PASTE), transposon-mediated gene editing, or other suitable gene editing or gene incorporation technology packaged into a single nucleic acid construct.

### 1. CROSS-REFERENCE TO RELATED APPLICATIONS

This application claims the benefit of and priority to U.S. Provisional Application No. 63/274,483, filed on Nov. 1, 2021; U.S. Provisional Application No. 63/282,055, filed on Nov. 22, 2021; U.S. Provisional Application No. 63/298,941, filed on Jan. 12, 2022; U.S. Provisional Application No. 63/318,344, filed on Mar. 9, 2022; and U.S. Provisional Application No. 63/352,897, filed on Jun. 16, 2022, each of which is hereby incorporated by reference in its entirety.

### 2. SEQUENCE LISTING

The instant application contains a Sequence Listing with 559 sequences, which has been submitted electronically in XML format and is hereby incorporated herein by reference in its entirety. Said XML copy, created on Oct. 31, 2022, is named 50408WO_CRF_sequencelisting.xml, and is 789,348 bytes in size.

### 3. BACKGROUND OF THE INVENTION

Programmable, efficient, and multiplexed genome integration of large, diverse DNA cargo independent of DNA repair remains an unsolved challenge of genome editing. Current gene integration approaches require double strand breaks that evoke DNA damage responses and rely on repair pathways that are inactive in terminally differentiated cells. Furthermore, CRISPR-based approaches that bypass double stranded breaks, such as Prime editing, are limited to modification or insertion of short sequences.

There is a need in the art for techniques which address and overcome these shortcomings and enable the insertion and/or deletion of large sequences into cells for therapeutic and circuit-based uses for broad purposes, across eukaryotic as well as prokaryotic systems.

### 4. SUMMARY OF THE INVENTION

A single nucleic acid construct is described herein that allows for incorporation of any template into any DNA locus using DNA delivery of a single component DNA. Additionally, a physical portion of the nucleic acid construct is capable of self-circularizing, forming a circular construct that contains a DNA template. Further, the nucleic acid construct can be packaged and delivered in any viral or non-viral delivery vector including a recombinant adenovirus, helper dependent adenovirus, AAV, HSV, annelovirus, retrovirus, lentivirus, Doggybone™ DNA (dbDNA™), minicircle, plasmid, miniDNA, LNP, or nanoplasmid. Delivery of the nucleic acid construct can also be by fusosome or exosome, (See, e.g., WO2019222403 which is incorporated by reference herein). Delivery of nucleic acid construct can also be by VesiCas (See, e.g., US20210261957A1 which is incorporated by reference herein).

The present disclosure provides nucleic acid compositions, methods, and an overall platform for site-specific genetic engineering using Programmable Addition via Site-Specific Targeting Elements (PASTE) (see Ionnidi et al.; doi: 10.1101/2021.11.01.466786; the entirety of which is incorporated herein by reference), transposon-mediated gene editing, or other suitable gene editing or gene incorporation technology packaged into a single nucleic acid construct, (described in some instances as an “installer”). Non-limiting examples of PASTE include those as described in U.S. Patent Publication No. 2022/0154224, which is herein incorporated by reference in its entirety. Described herein are “installer” nucleic constructs that encode for a prime editor system or a gene writer protein, one or more attachment site-containing guide RNA (atgRNA), optionally a nickase guide RNA (ngRNA), an integrase, a nucleic acid cargo, and optionally a recombinase. The integrase may be directly linked, for example by a peptide linker, to the prime editor fusion or gene writer protein. The nucleic acid construct described herein can be used to introduce, delete, or delete and introduce large pieces of DNA (as well as small pieces of DNA) to any genomic site in any organism. The technology described herein can be used broadly in therapeutic, diagnostic, agricultural, research, and for the general inclusion of genetic- and protein-based circuits.

In one aspect, this disclosure features a nucleic acid construct comprising: a nucleotide sequence encoding a prime editor system; a nucleotide sequence encoding at least a first attachment site-containing guide RNA (atgRNA); a nucleotide sequence encoding at least a first integrase; a nucleic acid cargo; optionally, a nucleotide sequence encoding a nickase guide RNA (ngRNA); and optionally a nucleotide sequence encoding a recombinase.

In some embodiments, the prime editor system comprises a nucleotide sequence encoding a nickase and a nucleotide sequence encoding a reverse transcriptase.

In some embodiments, the nucleotide sequence encoding the nickase and the nucleotide sequence encoding the reverse transcriptase are positioned in the construct such that when expressed the gene editor system comprises a fusion protein comprising the nickase and the reverse transcriptase.

In some embodiments, the first integrase that is encoded by a nucleotide sequence in the nucleic acid construct is fused to the prime editor system, the nickase, or the reverse transcriptase by a linker.

In some embodiments, the first atgRNA comprises a domain that is capable of guiding the prime editor system to a target sequence; and a reverse transcriptase (RT) template that comprises at least a portion of a first integration recognition site.

In some embodiments, the RT template comprises the entirety of the first integration recognition site.

In some embodiments, upon introducing the nucleic acid construct into a cell, the first atgRNA incorporates the first integrase recognition site into the cell's genome at the target sequence.

In some embodiments, the nucleic acid construct further comprises a second atgRNA.

In some embodiments, the first atgRNA and the second atgRNA are an at least first pair of atgRNAs, wherein the at least first pair of atgRNAs have domains that are capable of guiding the prime editor system to a target sequence, the first atgRNA further includes a first RT template that comprises at least a portion of the first integration recognition site; and the second atgRNA further includes a second RT template that comprises at least a portion of the first integration recognition site, and the first atgRNA and the second atgRNAs collectively encode the entirety of the first integration recognition site.

In some embodiments, upon introducing the nucleic acid construct into a cell, the first pair of atgRNAs incorporate the first integrase recognition site into the cell's genome at the target sequence.

In some embodiments, the nucleic acid construct further comprises a second integrase recognition site.

In some embodiments, the second integrase recognition site and the first integrase recognition site are a first cognate pair.

In some embodiments, nucleic acid construct further comprises a third integrase recognition site.

In some embodiments, the nucleic acid construct further comprises a fourth integrase recognition site.

In some embodiments, the third integrase recognition site and the fourth integrase recognition site are a second cognate pair.

In some embodiments, the second cognate pair has a faster integration rate than the first cognate pair, whereby in the presence of the first integrase the second cognate pair recombines prior to recombination of the first cognate pair.

In some embodiments, the nucleic acid construct further comprises a nucleotide sequence encoding a second integrase.

In some embodiments, the first integrase, the second integrase, or both, are selected from B×B1, Bcec, Sscd, Sacd, Int10, or Pa01.

In some embodiments, the first integrase and the second integrase recognize different integration recognition sites.

In some embodiments, the nucleic acid construct further comprises at least a first recombinase recognition site.

In some embodiments, the nucleic acid construct further comprises a second recombinase recognition site.

In some embodiments, the recombinase is FLP or Cre.

In some embodiments, the nucleic acid cargo comprises at least one of the following: a gene, an expression cassette, a logic gate system, or any combination thereof.

In some embodiments, the nucleic acid construct further comprises a sub-sequence of the nucleic acid construct that is capable of self-circularizing to form a self-circular nucleic acid.

In some embodiments, the sub-sequence of the nucleic acid construct that is capable of self-circularizing includes the nucleic acid cargo, whereby upon self-circularizing the self-circular nucleic acid comprises the nucleic acid cargo.

In some embodiments, the sub-sequence is flanked by the third integrase recognition site and the fourth integrase recognition site.

In some embodiments, the sub-sequence includes the second integrase recognition site.

In some embodiments, self-circularizing is mediated by recombination of the third integrase recognition site and the fourth integration recognition site by the first integrase.

In some embodiments, the sub-sequence is flanked by the first recombinase recognition site and the second recombinase recognition site.

In some embodiments, self-circularizing is mediated by recombination of the first recombinase recognition site and a second recombinase recognition site by the recombinase.

In some embodiments, the self-circular nucleic acid comprises one or more additional integration recognition sites that enable integration of additional nucleic acid cargo.

In some embodiments, upon introducing the nucleic acid construct into a cell and after self-circularizing to form the self-circular nucleic acid, the self-circular nucleic acid comprising the second integrase recognition site is capable of being integrated into the cell's genome at the target sequence that contains the first integrase recognition site.

In some embodiments, self-circularization to form the self-circular nucleic acid is effected by the first integrase and integration of the self-circular nucleic acid is effected by the second integrase.

In some embodiments, the nucleic acid construct further comprises a 5′ inverted terminal repeat (ITR).

In some embodiments, the nucleic acid construct further comprises a 3′ inverted terminal repeat (ITR).

In another aspect, this disclosure features a vector comprising any of the nucleic acid constructs described herein.

In some embodiments, the vector is recombinant adenovirus, helper dependent adenovirus, AAV, lentivirus, HSV, annelovirus, retrovirus, Doggybone™ DNA (dbDNA™), minicircle, plasmid, miniDNA, or nanoplasmid.

In another aspect, this disclosure features a pharmaceutical composition comprising any of the nucleic acid constructs described herein or any of the vectors described herein.

In another aspect, this disclosure features a method comprising administering an effective amount of any of the pharmaceutical compositions described herein to a patient in need thereof.

### 5. BRIEF DESCRIPTION OF THE DRAWINGS

These and other features, aspects, and advantages of the present invention will become better understood with regard to the following description, and accompanying drawings, where:

FIG. 1 illustrates a single construct that contains a prime editor fusion protein or gene writer protein, the attachment site-containing guide RNA (atgRNA), a nickase guide RNA (ngRNA), an integrase, a recombinase, recombination target sites, integration target site, a DNA of interest, and flanking ITRs. Recombinase expression leads to self-circularization of a sub-sequence of the single nucleic acid construct. DNA of interest contained within the self-circularized nucleic acid is capable of being integrated into a genomic locus of interest via an integrase.

FIG. 2 illustrates a single construct that contains a prime editor fusion protein or gene writer protein, the attachment site-containing guide RNA (atgRNA), a nickase guide RNA (ngRNA), an integrase, integration target sites, a DNA of interest, and flanking ITRs. Integrase expression leads to self-circularization of a subsequence of the single nucleic acid construct. Optionally, the integrase may be directly linked or fused to the prime editor protein or Gene Writer and expression driven from a single promoter. Self-circularization occurs at an integrase recognition target sequence (attB2/attP2). Additionally, a DNA of interest contained within the self-circularized nucleic acid is capable of being integrated into a genomic locus of interest via the integrase at an orthogonal integration target site (i.e., cognate pairs (e.g., attP1/attB1)). Initial self-circularization, prior to genomic integration, is achieved via the use of att integrase recognition target sites (i.e., attB2/attP2 and attP1/attB1) that are cognate pairs. The orthogonal integrase sites display an integrase-mediated recombination rate difference to allow for template/cargo circularization prior to genomic integration.

FIGS. 3A-3E show multiplex and orthogonal gene insertion with PASTE. FIG. 3A shows a schematic of AttP mutations tested for improving integration efficiency (SEQ ID NOS 394 and 540-542, respectively, in order of appearance). FIG. 3B shows integration efficiencies of wildtype and mutant AttP sites across a panel of AttB lengths. FIG. 3C shows a schematic of multiplexed integration of different cargo sets at specific genomic loci. Three fluorescent cargos (GFP, mCherry, and YFP) are inserted orthogonally at three different loci (ACTB, LMNB1, NOLC1) for in-frame gene tagging. FIG. 3D shows orthogonality of top 4 AttB/AttP dinucleotide pairs evaluated for GFP integration with PASTE at the ACTB locus. FIG. 3E shows efficiency of multiplexed PASTE insertion of combinations of fluorophores at ACTB, LMNB1, and NOLC1 loci. Data are mean (n=3)±s.e.m.

FIGS. 4A-4E show additional characterization of AttP mutants for improved editing and multiplexing. FIG. 4A shows AttP single mutants are characterized for PASTE EGFP integration at the ACTB locus. FIG. 4B shows characterization of integration of a 5 kb payload at the ACTB locus with all 16 possible dinucleotides for AttB/AttP pairs between the atgRNA and minicircle. FIG. 4C shows a schematic of the pooled AttB/AttP dinucleotide orthogonality assay. Each AttB dinucleotide sequence is cotransfected with a barcoded pool of all 16 AttP dinucleotide sequences and BxbINT, and relative integration efficiencies are determined by next generation sequencing of barcodes. All 16 AttB dinucleotides are profiled in an arrayed format with AttP pools. FIG. 4D illustrates relative insertion preferences for all possible AttB/AttP dinucleotide pairs determined by the pooled orthogonality assay. FIG. 4E shows orthogonality of BxbINT dinucleotides as measured by a pooled reporter assay. Each web logo motif shows the relative integration of different AttP sequences in a pool at a denoted AttB sequence with the listed dinucleotide.

FIG. 5 illustrates a schematic of single atgRNA and dual atgRNA approaches for beacon placement.

FIG. 6 illustrates the six different C-terminus to N-terminus arrangements (C-to-N) of exemplary nucleic acid programmable DNA binding proteins (napDNAbp), the RT, and the integrase is be fused or linked.

FIG. 7 illustrates the extrachromosomal circular DND (EccDNA) sensor assay to detect template circularization, beacon placement, and gene insertion. AttP (GT) for genome insertion. AttB′-AG and AttP′-AG at both ends for circularization in presence of Bxb1. EF1a promoter will drive NanoLuc and GFP expression. Screen for efficient di-nucleotides and configuration. Based on FG- and HD-AdV vector, tested in plasmid and virus format Abbreviations: Nanoluc=Nanoluc luciferase; GFP=green fluorescent protein; EF1α=elongation factor 1 alpha promoter; ori=origin of replication; and AmpR=gene encoding an Ampicillin resistance protein.

FIG. 8 illustrates transfection screening conditions for circularization detection and ACTB beacon placement and gene insertion.

FIG. 9 illustrates EccDNA ddPCR analysis.

FIG. 10 illustrates EccDNA ddPCR analysis with PE2, atgRNA, ngRNA components co-transfected.

FIG. 11 illustrates ACTB beacon placement analysis.

FIG. 12 illustrates EccDNA ACTB gene insertion analysis at a placed beacon.

FIG. 13 illustrates transfection screening conditions for circularization detection and LMNB beacon placement and gene insertion.

FIG. 14 illustrates in cell EccDNA circularization detection by GFP detection.

FIG. 15 illustrates EccDNA ddPCR analysis.

FIG. 16 illustrates EccDNA LMNB beacon placement analysis.

FIG. 17 illustrates LMNB gene insertion analysis at a placed beacon.

FIG. 18 illustrates a single construct that contains a prime editor fusion protein, dual attachment site-containing guide RNA (atgRNAs) (i.e., atgF and atgR), a tet-inducible integrase, an integration target site, a DNA of interest, and flanking ITRs. Abbreviations: ITR=inverted terminal repeat; Ad5 v=Adenovirus 5 packaging domain; atgR=atgRNA reverse; U6=U6 promoter; atgF=atgRNA forward; U6=U6 promoter; PE2=prime editing complex PE2 (as described herein); tet-off=tetracyline off promoter; EF1a=elongation factor 1 alpha promoter; mScarlet=a red fluorescent protein; Nanoluc=Nanoluc luciferase; GFP=green fluorescent protein; ori=origin of replication; and AmpR=gene encoding an Ampicillin resistance protein.

FIGS. 19A-19J show brightfield (FIGS. 19A, 19C, 19E, 19G, and 19I) and RFP (FIGS. 19B, 19D, 19F, 19H, and 19J) on day 2 following transfection with the single nucleic acid construct depicted in FIG. 18.

FIGS. 20A-20B illustrates beacon placement (BP) at the Nolc1 locus. FIG. 20A shows raw data from a ddPCR assay at the Nolc1 locus. FIG. 20B shows summary of the data in FIG. 20A. Abbreviation: AIO-all-in-one (also referred to herein as the single nucleic acid construct).

FIGS. 21A-21B illustrates programmable gene insertion (PGI) at the Nolc1 locus. FIG. 21A shows raw data from a ddPCR assay at the Nolc1 locus. FIG. 21B shows summary of the data in FIG. 21A. Abbreviation: AIO-all-in-one (also referred to herein as the single nucleic acid construct).

FIG. 22 shows PGI conversion rate (=PGI %/(PGI%+BP %)) for the data in FIGS. 20A-20B and FIGS. 21A-21B.

FIGS. 23A-23B show next generation sequence data confirming beacon placement and PGI. FIG. 23A shows next generation sequencing data for beacon placement. FIG. 23B shows next generation sequencing data for PGI.

FIG. 24 shows next generation sequence data from FIG. 22A and FIG. 22B as PGI conversion rate (=PGI %/(PGI %+BP %)).

FIGS. 25A-25L show brightfield (FIG. 25A-25D), RFP (FIG. 25E-25H), and GFP (FIG. 251-25L) on day 2 following transection with the single nucleic acid construct depicted in FIG. 18 or a four plasmid system.

FIGS. 26A-26B illustrates beacon placement (BP) at the human factor IX (“hF9”) locus. FIG. 26A shows raw data from a ddPCR assay at the hF9 locus. FIG. 26B shows summary of the data in FIG. 26A. Abbreviation: AIO-all-in-one (also referred to herein as the single nucleic acid construct).

FIGS. 27A-27B illustrates programmable gene insertion (PGI) at the hl P locus. FIG. 27A shows raw data from a ddPCR assay at the hF9 locus. FIG. 27B shows summary of the data in FIG. 27A. Abbreviation: AIO-all-in-one (also referred to herein as the single nucleic acid construct).

### 6. DETAILED DESCRIPTION OF THE INVENTION

### 6.1. Gene Editors

Unless defined otherwise, all technical and scientific terms used herein have the meaning commonly understood by a person skilled in the art to which this invention belongs. As used herein, the following terms have the meanings ascribed to them below.

“Gene editor” as used herein, is a protein that that can be used to perform gene editing, gene modification, gene insertion, gene deletion, or gene inversion. Such an enzyme or enzyme fusion may contain DNA or RNA targetable nuclease protein (i.e., Cas protein, ADAR, or ADAT), wherein target specificity is mediated by a complexed nucleic acid (i.e., guide RNA). Such an enzyme or enzyme fusion may be a DNA/RNA targetable protein, wherein target specificity is mediated by internal, conjugated, fused, or linked amino acids, such as within TALENs, ZFNs, or meganucleases. The skilled person in the art would appreciate that the gene editor can demonstrate targeted nuclease activity, targeted binding with no nuclease activity, or targeted nickase activity (or cleavase activity). A gene editor comprising a targetable protein may be fused or linked to one or more proteins or protein fragment motifs. Gene editors may be fused, linked, complexed, operate in cis or trans to one or more integrase, recombinase, polymerase, telomerase, reverse transcriptase, or invertase. A gene editor can be a prime editor fusion protein or a gene writer fusion protein.

“Prime editor fusion protein” as used herein, describes a protein that is used in prime editing. “Prime editor system” as used herein, describes the components used in prime editing. Prime editing uses CRISPR enzyme that nicks or cuts only single strand of double stranded DNA, i.e., a nickase; the nickase can occur either naturally or by mutation or modification of a nuclease that makes double stranded cuts. Such an enzyme can be a catalytically-impaired Cas9 endonuclease (a nickase). Such an enzyme can be a Casl2a/b, MAD7, or variant thereof. The nickase is fused to an engineered reverse transcriptase (RT). The nickase is programmed (directed) with a prime-editing guide RNA (pegRNA). The skilled person in the art would appreciate that the pegRNA both specifies the target site and encodes the desired edit. Described herein, are attachment site-containing guide RNA (atgRNA) that both specify the target and encode for the desired integrase target recognition site. The nickase may be programmed (directed) with an atgRNA. Advantageously the nickase is a catalytically-impaired Cas9 endonuclease, a Cas9 nickase, that is fused to the reverse transcriptase. During genetic editing, the Cas9 nickase part of the protein is guided to the DNA target site by the pegRNA (or atgRNA), whereby a nick or single stranded cut occurs. The reverse transcriptase domain then uses the pegRNA (or atgRNA) to template reverse transcription of the desired edit, directly polymerizing DNA onto the nicked target DNA strand. The edited DNA strand replaces the original DNA strand, creating a heteroduplex containing one edited strand and one unedited strand. Afterward, optionally, the prime editor (PE) guides resolution of the heteroduplex to favor copying the edit onto the unedited strand, completing the process (typically achieved with a nickase gRNA). Other enzymes that can be used to nick or cut only a single strand of double stranded DNA includes a cleavase (e.g., cleavase I enzyme).

In some embodiments, an additional agent or agents may be added that improve the efficiency and outcome purity of the prime edit. In some embodiments, the agent may be chemical or biological and disrupt DNA mismatch repair (MMR) processes at or near the edit site (i.e., PE4 and PE5 and PEmax architecture by Chen et al. Cell, 184, 1-18, Oct. 28, 2021; Chen et al. is incorporated herein by reference). In typical embodiments, the agent is a MMR-inhibiting protein. In certain embodiments, the MMR-inhibiting protein is dominant negative MMR protein. In certain embodiments, the dominant negative MMR protein is MLH1dn. In particular embodiments, the MMR-inhibiting agent is incorporated into the single nucleic acid construct design described herein. In some embodiments, the MMR-inhibiting agent is linked or fused to the prime editor protein fusion, which may or may not have a linked or fused integrase. In some embodiments, the MMR-inhibiting agent is linked or fused to the Gene Writer™ protein, which may or may not have a linked or fused integrase.

The prime editor or gene editor system can be used to achieve DNA deletion and replacement. In some embodiments, the DNA deletion replacement is induced using a pair of pegRNA or atgRNAs that target opposite DNA strands, programming not only the sites that are nicked but also the outcome of the repair (i.e., PrimeDel by Choi et al. Nat. Biotechnology, Oct. 14, 2021; Choi et al. is incorporated herein by reference and TwinPE by Anzalone et al. BioRxiv, Nov. 2, 2021; Anzalone et al. is incorporated herein by reference). In some embodiments described herein, the DNA deletion is induced using a single atgRNA. In some embodiments, the DNA deletion and replacement is induced using a wild type Cas9 prime editor (PE-Cas9) system (i.e., PEDAR by Jiang et al. Nat. Biotechnology, Oct. 14, 2021; Jiang et al. is incorporated herein by reference) In some embodiments, the DNA replacement is an integrase target recognition site or recombinase target recognition site. In certain embodiments, the constructs and methods described herein may be utilized to incorporate the pair of pegRNAs used in PrimeDel, TwinPE (WO2021226558 incorporated by reference herein), or PEDAR, the prime editor fusion protein or Gene Writer protein, optionally a nickase guide RNA (ngRNA), an integrase, a nucleic acid cargo, and optionally a recombinase into a single nucleic acid construct described herein. The integrase may be directly linked, for example by a peptide linker, to the prime editor fusion or gene writer protein.

In some embodiments, the prime editors can refer to a retrovirus or lentivirus reverse transcriptase such as a Moloney Murine Leukemia Virus (M-MLV) reverse transcriptase (RT) fused to a CRISPR enzyme nickase such as a Cas9 H840A nickase, a Cas9nickase. In some embodiments, the prime editors can refer to a retrovirus or lentivirus reverse transcriptase such as a Moloney Murine Leukemia Virus (M-MLV) reverse transcriptase (RT) fused to a cleavase. In some embodiments the RT can be fused at, near or to the C-terminus of a Cas9nickase, e.g., Cas9 H840A. Fusing the RT to the C-terminus region, e.g., to the C-terminus, of the Cas9 nickase may result in higher editing efficiency. Such a complex is called PEI. In some embodiments, the CRISPR enzyme nickase, e.g., Cas9 (H840A), i.e., a Cas9nickase, can be linked to a non-M-MLV reverse transcriptase such as an AMV-RT or XRT (Cas9 (H840A)-AMV-RT or XRT). In some embodiments, instead of the CRISPR enzyme nickase being a Cas9 (H840A), i.e., instead of being a Cas9 nickase, the CRISPR enzyme nickase instead can be a CRISPR enzyme that naturally is a nickase or cuts a single strand of double stranded DNA; for instance, the CRISPR enzyme nickase can be Cas12a/b. Alternatively, the CRISPR enzyme nickase can be another mutation of Cas9, such as Cas9 (D10A). A CRISPR enzyme, such as a CRISPR enzyme nickase, such as Cas9 (wild type), Cas9 (H840A), Cas9 (D10A) or Cas 12a/b nickase can be fused in some embodiments to a pentamutant of M-MLV RT (D200N/L603W/T330P/T306K/W313F), whereby there can be up to about 45-fold higher efficiency, and this is called PE2. In some embodiments, the M-MLV RT comprise one or more of the mutations Y8H, P51L, S56A, S67R, E69K, VI29P, L139P, T197A, H204R, V223H, T246E, N249D, E286R, Q2911, E302K, E302R, F309N, M320L, P330E, L435G, L435R, N454K, D524A, D524G, D524N, E562Q, D583N, H594Q, E607K, D653N, and L671P. Specific M-MLV RT mutations are shown in Table 1.

TABLE 1

| SEQ ID | | Forward Sequence |
|---|---|---|
| NO | Description | (5′-3′) |
| SEQ ID | RT_mut_ | ttgagcgggCCCccaccgt |
| NO: 01 | L139P | |
| SEQ ID | RT_mut_ | cagcgggctCAGctgatagca |
| NO: 02 | E562Q | |
| SEQ ID | RT_mut_ | cggatggctAACcaagcggcc |
| NO: 03 | D653N | |

In some embodiments, the reverse transcriptase can also be a wild-type or modified transcription xenopolymerase (RTX), avian myeloblastosis virus reverse transcriptase (AMV RT), Feline Immunodeficiency Virus reverse transcriptase (FIV-RT), FeLV-RT (Feline leukemia virus reverse transcriptase), HIV-RT (Human Immunodeficiency Virus reverse transcriptase). In some embodiments, the reverse transcriptase can be a fusion of MMuLV to the Sto7d DNA binding domain (see Ionnidi et al.; https://doi.org/10.1101/2021.11.01.466786). The fusion of MMuL V to the Sto7d DNA binding domain sequence is given in Table 2.

TABLE 2

| | | SEQ |
|---|---|---|
| Descrip- | | ID |
| tion | Forward Sequence (5′-3′) | NO: |
| RT(1- | atgactcactatcag | 4 |
| 478)_ | gccttgcttttggacacggaccgg | |
| Sto7d | gtccagttcggaccggtggtagcc | |
| fusion | ctgaacccggctacgctgctccca | |
| [MMulv | ctgcctgaggaagggctgcaacac | |
| se- | aactgccttgatGGGACAGGTGGC | |
| quence | GGTGGTGTCACCGTCAAGTTCAAG | |
| (in | TACAAGGGTGAGGAACTTGAAGTT | |
| bold), | GATATTAGCAAAATCAAGAAGGTT | |
| Sto7d | TGGCGCGTTGGTAAAATGATATCT | |
| se- | TTTACTTATGACGACAACGGCAAG | |
| quence] | ACAGGTAGAGGGGCAGTGTCTGAG | |
| | AAAGACGCCCCCAAGGAGCTGTTG | |
| | CAAATGTTGGAAAAGTCTGGGAAA | |
| | AAGtctggcggctcaaaaagaacc | |
| | gccgacggcagcgaattcgagccc | |
| | aagaagaagaggaaagtc | |

PE3, PE3b, PE4, PE5, and/or PEmax, which a skilled person can incorporate into the gene editor (and express from a single nucleic acid construct, e.g., any of the single nucleic acid constructs described herein), involves nicking the non-edited strand, potentially causing the cell to remake that strand using the edited strand as the template to induce HR. The nicking of the non-edited strand can involve the use of a nicking guide RNA (ngRNA).

The skilled person can readily incorporate into a gene editor single nucleic acid construct (“installer”) described herein a prime editing or CRISPR system. Examples of prime editors can be found in the following: WO2020/191153, WO2020/191171, WO2020/191233, WO2020/191234, WO2020/191239, WO2020/191241, WO2020/191242, WO2020/191243, WO2020/191245, WO2020/191246, WO2020/191248, WO2020/191249, each of which is incorporated by reference herein in its entirety. In addition, mention is made, and can be used herein, of CRISPR Patent Applications and Patents of the Zhang laboratory and/or Broad Institute, Inc, and Massachusetts Institute of Technology and/or Broad Institute, Inc., Massachusetts Institute of Technology and President and Fellows of Harvard College and/or Editas Medicine, Inc. Broad Institute, Inc., The University of Iowa Research Foundation and Massachusetts Institute of Technology, including those claiming priority to U.S. Application 61/736,527, filed Dec. 12, 2012, including U.S. Pat. Nos. 11,104,937, 11,091,798, 11,060,115, 11,041,173, 11,021,740, 11,008,588, 11,001,829, 10,968,257, 10,954,514, 10,946,108, 10,930,367, 10,876,100, 10,851,357, 10,781,444, 10,711,285, 10,689,691, 10,648,020, 10,640,788, 10,577,630, 10,550,372, 10,494,621, 10,377,998, 10,266,887, 10,266,886, 10,190,137, 9,840,713, 9,822,372, 9,790,490, 8,999,641, 8,993,233, 8,945,839, 8,932,814, 8,906,616, 8,895,308, 8,889,418, 8,889,356, 8,871,445, 8,865,406, 8,795,965, 8,771,945, and 8,697,359; CRISPR Patent Applications and Patents of the Doudna laboratory and/or of Regents of the University of California, the University of Vienna and Emmanuelle Charpentier, including those claiming priority to U.S, application 61/652,086, filed May 25, 2012, and/or 61/716,256, filed Oct. 19, 2012, and/or 61/757,640, filed Jan. 28, 2013, and/or 61/765,576, filed Feb. 15, 2013 and/or 13/842,859, including U.S. Pat. Nos. 11,028,412, 11,008,590, 11,008,589, 11,001,863, 10,988,782, 10,988,780, 10,982,231, 10,982,230, 10,900,054, 10,793,878, 10,774,344, 10,752,920, 10,676,759, 10,669,560, 10,640,791, 10,626,419, 10,612,045, 10,597,680, 10,577,631, 10,570,419, 10,563,227, 10,550,407, 10,533,190, 10,526,619, 10,519,467, 10,513,712, 10,487,341, 10,443,076, 10,428,352, 10,421,980, 10,415,061, 10,407,697, 10,400,253, 10,385,360, 10,358,659, 10,358,658, 10,351,878, 10,337,029, 10,308,961, 10,301,651, 10,266,850, 10,227,611, 10,113,167, and 10,000,772; CRISPR Patent Applications and Patents of Vilnius University and/or the Siksnys laboratory, including those claiming priority to U.S, application 62/046,384 and/or 61/625,420 and/or 61/613,373 and/or PCT/IB2015/056756, including U.S. Pat. No. 10,385,336; CRISPR Patent Applications and Patents of the President and Fellows of Harvard College, including those of George Church's laboratory and/or claiming priority to U.S, application 61/738,355, filed Dec. 17, 2012, including 11,111,521, 11,085,072, 11,064,684, 10,959,413, 10,925,263, 10,851,369, 10,787,684, 10,767,194, 10,717,990, 10,683,490, 10,640,789, 10,563,225, 10,435,708, 10,435,679, 10,375,938, 10,329,587, 10,273,501, 10,100,291, 9,970,024, 9,914,939, 9,777,262, 9,587,252, 9,267,135, 9,260,723, 9,074,199, 9,023,649; CRISPR Patent Applications and Patents of the President and Fellows of Harvard College, including those of David Liu's laboratory, including 11,111,472, 11, 104,967, 11,078,469, 11,071,790, 11,053,481, 11,046,948, 10,954,548, 10,947,530, 10,912,833, 10,858,639, 10,745,677, 10,704,062, 10,682,410, 10,612,011, 10,597,679, 10,508,298, 10,465,176, 10,323,236, 10,227,581, 10,167,457, 10,113,163, 10,077,453, 9,999,671, 9,840,699, 9,737,604, 9,526,784, 9,388,430, 9,359,599, 9,340,800, 9,340,799, 9,322,037, 9,322,006, 9,228,207, 9,163,284, and 9,068,179; and CRISPR Patent Applications and Patents of Toolgen Incorporated and/or the Kim laboratory and/or claiming priority to U.S, application 61/717,324, filed Oct. 23, 2012 and/or 61/803,599, filed Mar. 20, 2013 and/or 61/837,481, filed Jun. 20, 2013 and/or 62/033,852, filed Aug. 6, 2014 and/or PCT/KR2013/009488 and/or PCT/KR2015/008269, including U.S. Pat. Nos. 10,851,380, and 10,519,454; and CRISPR Patent Applications and Patents of Sigma and/or Millipore and/or the Chen laboratory and/or claiming priority to U.S, application 61/734,256, filed Dec. 6, 2012 and/or 61/758,624, filed Jan. 30, 2013 and/or 61/761,046, filed Feb. 5, 2013 and/or 61/794,422, filed Mar. 15, 2013, including U.S. Pat. No. 10,731,181, each of which is hereby incorporated herein by reference, and from the disclosures of the foregoing, the skilled person can readily make and use a prime editing or CRISPR system, and can especially appreciate impaired endonucleases, such as a mutated Cas9 that only nicks a single strand of DNA and is hence a nickase, or a CRISPR enzyme that only makes a single-stranded cut that can be employed in a PASTE system of the invention. Further, from the disclosures of the foregoing, the skilled person can incorporate the selected CRISPR enzyme, as part of the prime editor fusion or gene editor fusion, into a single nucleic acid construct (“installer”) described herein.

Prior to RT-mediated edit incorporation, the prime editor protein (1) site-specifically targets a genomic locus and (2) performs a catalytic cut or nick. These steps are typically performed by a CRISPR-Cas. However, in some embodiments the Cas protein may be substituted by other nucleic acid programmable DNA binding proteins (napDNAbp) such as zinc finger nucleases (ZFNs), transcription activator-like effector nucleases (TALENs), or meganucleases. In addition, to the extent the “targeting rules” of other napDNAbp are known or are newly determined, it becomes possible to use new napDNAbp, beyond Cas9, to site specifically target and modify genomic sites of interest.

Similar to a prime editor protein, a Gene Writer can introduce novel DNA elements, such as an integration target site, into a DNA locus. A Gene Writer protein comprises: (A) a polypeptide or a nucleic acid encoding a polypeptide, wherein the polypeptide comprises (i) a reverse transcriptase domain, and either (x) an endonuclease domain that contains DNA binding functionality or (y) an endonuclease domain and separate DNA binding domain; and (B) a template RNA comprising (i) a sequence that binds the polypeptide and (ii) a heterologous insert sequence. Examples of such Gene Writer™ proteins and related systems can be found in US20200109398, which is incorporated by reference herein in its entirety.

In some embodiments, the prime editor or Gene Writer protein fusion or prime editor protein linked or fused to an integrase is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more single nucleic acid constructs described herein.

In some embodiments, an integrase or recombinase is directly linked or fused, for example by a peptide linker, which may be cleavable or non-cleavable, to the prime editor fusion protein (i.e, fused Cas9 nickase-reverse transcriptase) or Gene Writer protein. Suitable linkers, for example between the Cas9, RT, and integrase, may be selected from Table 3:

TABLE 3

| | | SEQ | Amino | SEQ |
|---|---|---|---|---|
| | Sequence | ID | acid | ID |
| | (5′-3′) | NO: | sequence | NO: |
| A-P2A | GGAAGCGGAGC | 5 | GSGATNFSL | 13 |
| | TACTAACTTCA | | LKQAGDVEE | |
| | GCCTGCTGAAG | | NPGP | |
| | CAGGCTGGC | | | |
| | GACGTGGAGG | | | |
| | AGAACCCTGGA | | | |
| | CCT | | | |
| B- | GGGGGAGGAGG | 6 | GGGGSGGGG | 14 |
| (GGGS)3 | TTCTGGAGGCG | | SGGGGS | |
| | GAGG | | | |
| | CTCCGGAGGCG | | | |
| | GAGGGTCA | | | |
| C- | GGAGGTGGCGG | 7 | GGGGS | 15 |
| GGGGS | GAGC | | | |
| D- | CCCGCACCAGC | 8 | PAPAP | 16 |
| PAPAP | GCCT | | | |
| E- | GAGGCAGCTGC | 9 | EAAAKEAAA | 17 |
| (EAAAK) | CAAGGAAGCCG | | KEAAAK | |
| 3 | CTGCCAAGGAG | | | |
| | GCGGCCGCAAA | | | |
| | G | | | |
| F-XTEN | AGTGGGAGCGA | 10 | SGSETPGTS | 18 |
| | GACCCCTGGGA | | ESATPES | |
| | CTAGCGAGTCA | | | |
| | GCTACACCCGA | | | |
| | AAGC | | | |
| G- | GGGGGGTCAGG | 11 | GGSGGSGGS | 19 |
| (GGS)6 | TGGATCCGGCG | | GGSGGSGGS | |
| | GAAGTGGCGGA | | | |
| | TCCGGTGGATC | | | |
| | TGGCGGCAGT | | | |
| H- | GAAGCTGCTGC | 12 | EAAAK | 20 |
| EAAAK | TAAG | | | |
| (GGGGS) | GGCGGCGGCGG | 543 | GGGGSGGGG | 551 |
| 4 | CAGCGGCGGCG | | SGGGGSGGG | |
| | GCGGCAGCGGC | | GS | |
| | GGCGGCGGCAG | | | |
| | CGGCGGCGGCG | | | |
| | GCAGC | | | |
| PAS8 | GGCGGCGCGAG | 544 | GGASPAGG | 552 |
| | CCCGGCGGGCG | | | |
| | GC | | | |
| PAS12 | GGCGGCGCGAGC | 545 | GGASPAAPA | 553 |
| | CCGGCGGCGCCG | | PAG | |
| | GCGCCGGCGGGC | | | |
| A(EAAK) | GCGGAAGCGGCG | 546 | AEAAKEAAK | 554 |
| 4ALEA(E | AAAGAAGCGGCG | | EAAKEAAKA | |
| AAAK)4A | AAAGAAGCGGCG | | LEAEAAAKE | |
| | AAAGAAGCGGCG | | AAAKEAAAK | |
| | AAAGCGCTGGAA | | EAAAKA | |
| | GCGGAAGCGGCG | | | |
| | GCGAAAGAAGCG | | | |
| | GCGGCGAAAGAA | | | |
| | GCGGCGGCGAAA | | | |
| | GAAGCGGCGGCG | | | |
| | AAAGCG | | | |
| Camel | GCGCATCATAGC | 547 | AHHSEDPGG | 555 |
| | GAAGATCCGGGC | | GGSGGGGSG | |
| | GGCGGCGGCAGC | | GGGS | |
| | GGCGGCGGCGGC | | | |
| | AGCGGCGGCGGC | | | |
| | GGCAGC | | | |
| FRF | GGCGGCGGCGGC | 548 | GGGGSEAAA | 556 |
| | AGCGAAGCGGCG | | KGGGGS | |
| | GCGAAAGGCGGC | | | |
| | GGCGGCAGC | | | |
| RFR | GAAGCGGCGGCG | 549 | EAAAKGGGG | 557 |
| | AAAGGCGGCGGC | | SEAAAK | |
| | GGCAGCGAAGCG | | | |
| | GCGGCGAAA | | | |
| Modified | AGCGGCGGCAGC | 550 | SGGSSGGSS | 558 |
| XTEN | AGCGGCGGCAGC | | GSETPGTSE | |
| (mXTEN) | AGCGGCAGCGAA | | SATPESSGG | |
| | ACCCCGGGCACC | | SSGGSST | |
| | AGCGAAAGCGCG | | | |
| | ACCCCGGAAAGC | | | |
| | AGCGGCGGCAGC | | | |
| | AGCGGCGGCAGC | | | |
| | AGCACC | | | |

In some embodiments, the prime editor or Gene Writer protein fusion or prime editor protein linked or fused to an integrase is expressed as a split construct. In typical embodiments, the split construct in reconstituted in a cell. In some embodiments, the split construct can be fused or ligated via intein protein splicing. In some embodiments, the split construct can be reconstituted via protein-protein inter-molecular bonding and/or interactions. In some embodiments, the split construct can be reconstituted via chemical, biological, or environmental induced oligomerization. In certain embodiments, the split construct can be adapted into one or more nucleic acid constructs described herein.

### 6.2. Type II CRISPR Proteins

The skilled person can incorporate a selected CRISPR enzyme, described below, as part of the prime editor fusion, into a single nucleic acid construct (“installer”) described herein. Streptococcus pyogenes Cas9 (SpCas9), the most common enzyme used in genome-editing applications, is a large nuclease of 1368 amino acid residues. Advantages of SpCas9 include its short, 5′-NGG-3′ PAM and very high average editing efficiency. SpCas9 consists of two lobes: a recognition (REC) lobe and a nuclease (NUC) lobe. The REC lobe can be divided into three regions, a long a helix referred to as the bridge helix (residues 60-93), the REC1 (residues 94-179 and 308-713) domain, and the REC2 (residues 180-307) domain. The NUC lobe consists of the RuvC (residues 1-59, 718-769, and 909-1098), HNH (residues 775-908), and PAM-interacting (PI) (residues 1099-1368) domains. The negatively charged sgRNA: target DNA heteroduplex is accommodated in a positively charged groove at the interface between the REC and NUC lobes. In the NUC lobe, the RuvC domain is assembled from the three split RuvC motifs (RuvC I-III) and interfaces with the PI domain to form a positively charged surface that interacts with the 30 tail of the sgRNA. The HNH domain lies between the RuvC II-III motifs and forms only a few contacts with the rest of the protein. Structural aspects of SpCas9 are described by Nishimasu et al., Crystal Structure of Cas9 in Complex with Guide RNA and Target DNA, Cell 156, 935-949, Feb. 27, 2014.

REC lobe: The REC lobe includes the REC1 and REC2 domains. The REC2 domain does not contact the bound guide: target heteroduplex, indicating that truncation of REC lobe may be tolerated by SpCas9. Further, SpCas9 mutant lacking the REC2 domain (D175-307) retained ˜50% of the wild-type Cas9 activity, indicating that the REC2 domain is not critical for DNA cleavage. In striking contrast, the deletion of either the repeat-interacting region (D97-150) or the anti-repeat-interacting region (D312-409) of the REC1 domain abolished the DNA cleavage activity, indicating that the recognition of the repeat: anti-repeat duplex by the REC1 domain is critical for the Cas9 function.

PAM-Interacting domain: The NUC lobe contains the PAM-interacting (PI) domain that is positioned to recognize the PAM sequence on the noncomplementary DNA strand. The PI domain of SpCas9 is required for the recognition of 5′-NGG-3′ PAM, and deletion of the PI domain (41099-1368) abolished the cleavage activity, indicating that the PI domain is critical for SpCas9 function and a major determinant for the PAM specificity.

RuvC′ domain: The RuvC nucleases of SpCas9 have an RNase H fold and four catalytic residues, Asp10 (Ala), Glu762, His983, and Asp986, that are critical for the two-metal cleavage of the noncomplementary strand of the target DNA. In addition to the conserved RNase H fold, the Cas9 RuvC domain has other structural elements involved in interactions with the guide: target heteroduplex (an end-capping loop between a42 and a43) and the PI domain/stem loop 3 (β hairpin formed by β3 and β4).

HNH domain: SpCas9 HNH nucleases have three catalytic residues, Asp839, His840, and Asn863 and cleave the complementary strand of the target DNA through a single-metal mechanism.

sgRNA: DNA recognition: The sgRNA guide region is primarily recognized by the REC lobe. The backbone phosphate groups of the guide region (nucleotides 2, 4-6, and 13-20) interact with the REC1 domain (Arg165, Glyl66, Arg403, Asn407, Lys510, Tyr515, and Arg661) and the bridge helix (Arg63, Arg66, Arg70, Arg71, Arg74, and Arg78). The 20-hydroxyl groups of G1, C15, U16, and G19 hydrogen bond with Val1009, Tyr450, Arg447/Ile448, and Thr404, respectively.

A mutational analysis demonstrated that the R66A, R70A, and R74A mutations on the bridge helix markedly reduced the DNA cleavage activities, highlighting the functional significance of the recognition of the sgRNA “seed” region by the bridge helix. Although Arg78 and Arg165 also interact with the “seed” region, the R78A and R165A mutants showed only moderately decreased activities. These results are consistent with the fact that Arg66, Arg70, and Arg74 form multiple salt bridges with the sgRNA backbone, whereas Arg78 and Arg165 form a single salt bridge with the sgRNA backbone. Moreover, the alanine mutations of the repeat: anti-repeat duplex-interacting residues (Arg75 and Lys163) and the stemloop-1-interacting residue (Arg69) resulted in decreased DNA cleavage activity, confirming the functional importance of the recognition of the repeat: anti-repeat duplex and stem loop 1 by Cas9.

RNA-guided DNA targeting: SpCas9 recognizes the guide: target heteroduplex in a sequence-independent manner. The backbone phosphate groups of the target DNA (nucleotides 1, 9-11, 13, and 20) interact with the REC1 (Asn497, Trp659, Arg661, and Gln695), RuvC (Gln926), and PI (Glu1108) domains. The C2′ atoms of the target DNA (nucleotides 5, 7, 8, 11, 19, and 20) form van der Waals interactions with the REC1 domain (Leu169, Tyr450, Met495, Met694, and His698) and the RuvC domain (Ala728). The terminal base pair of the guide: target heteroduplex (G1: C20′) is recognized by the RuvC domain via end-capping interactions; the sgRNA G1 and target DNA C20′ nucleobases interact with the Tyr1013 and Val1015 side chains, respectively, whereas the 20-hydroxyl and phosphate groups of sgRNA G1 interact with Val1009 and Gln926, respectively.

Repeat: Anti-Repeat duplex recognition: The nucleobases of U23/A49 and A42/G43 hydrogen bond with the side chain of Arg1122 and the main-chain carbonyl group of Phe351, respectively. The nucleobase of the flipped U44 is sandwiched between Tyr325 and His328, with its N3 atom hydrogen bonded with Tyr325, whereas the nucleobase of the unpaired G43 stacks with Tyr359 and hydrogen bonds with Asp364.

The nucleobases of G21 and U50 in the G21: U50 wobble pair stack with the terminal C20: G10 pair in the guide: target heteroduplex and Tyr72 on the bridge helix, respectively, with the U50 04 atom hydrogen bonded with Arg75. Notably, A51 adopts the syn conformation and is oriented in the direction opposite to U50. The nucleobase of A51 is sandwiched between Phe1105 and U63, with its N1, N6, and N7 atoms hydrogen bonded with G62, Glyl103, and Phe1 105, respectively.

Stem-loop recognition: Stem loop 1 is primarily recognized by the REC lobe, together with the PI domain. The backbone phosphate groups of stem loop 1 (nucleotides 52, 53, and 59-61) interact with the REC1 domain (Leu455, Ser460, Arg467, Thr472, and Ile473), the PI domain (Lys1123 and Lys1124), and the bridge helix (Arg70 and Arg74), with the 20-hydroxyl group of G58 hydrogen bonded with Leu455. A52 interacts with Phe1 105 through a face-to-edge p-p stacking interaction, and the flipped U59 nucleobase hydrogen bonds with Asn77.

The single-stranded linker and stem loops 2 and 3 are primarily recognized by the NUC lobe. The backbone phosphate groups of the linker (nucleotides 63-65 and 67) interact with the RuvC domain (Glu57, Lys742, and Lys1097), the PI domain (Thr1102), and the bridge helix (Arg69), with the 20-hydroxyl groups of U64 and A65 hydrogen bonded with Glu57 and His721, respectively. The C67 nucleobase forms two hydrogen bonds with Val1100.

Stem loop 2 is recognized by Cas9 via the interactions between the NUC lobe and the non-Watson-Crick A68: G81 pair, which is formed by direct (between the A68 N6 and G81 06 atoms) and water-mediated (between the A68 Nl and G81 N1 atoms) hydrogen-bonding interactions. The A68 and G81 nucleobases contact Ser1351 and Tyr1356, respectively, whereas the A68: G81 pair interacts with Thr1358 via a water-mediated hydrogen bond. The 20-hydroxyl group of A68 hydrogen bonds with His1349, whereas the G81 nucleobase hydrogen bonds with Lys33.

Stem loop 3 interacts with the NUC lobe more extensively, as compared to stem loop 2. The backbone phosphate group of G92 interacts with the RuvC domain (Arg40 and Lys44), whereas the G89 and U90 nucleobases hydrogen bond with Gln1272 and Glu1225/Ala1227, respectively. The A88 and C91 nucleobases are recognized by Asn46 via multiple hydrogen-bonding interactions.

Cas9 proteins smaller than SpCas9 allow more efficient packaging of nucleic acids encoding CRISPR systems, e.g., Cas9 and sgRNA into one rAAV (“all-in-one-AAV”) particle. In addition, efficient packaging of CRISPR systems can be achieved in other viral vector systems (i.e., lentiviral, hd-AAV, etc.) and non-viral vector systems (i.e., lipid nanoparticle). Small Cas9 proteins can be advantageous for multidomain-Cas-nuclease-based systems for prime editing. Well characterized smaller Cas9 proteins include Staphylococcus aureus (SauCas9, 1053 amino acid residues) and Campylobacter jejuni (CjCas9, 984 amino residues). However, both recognize longer PAMs, 5′-NNGRRT-3′ for SauCas9 (R=A or G) and 5′-NNNNRYAC-3′ for CjCas9 (Y=C or T), which reduces the number of uniquely addressable target sites in the genome, in comparison to the NGG SpCas9 PAM. Among smaller Cas9s, Schmidt et al. identified Staphylococcus lugdunensis (Slu) Cas9 as having genome-editing activity and provided homology mapping to SpCas9 and SauCas9 to facilitate generation of nickases and inactive (“dead”) enzymes (Schmidt et al., 2021, Improved CRISPR genome editing using small highly active and specific engineered RNA-guided nucleases. Nat Commun 12, 4219. doi.org/10.1038/s41467-021-24454-5) and engineered nucleases with higher cleavage activity by fragmenting and shuffling Cas9 DNAs. The small Cas9s and nickases are useful in the instant invention.

Besides dead Cas9 and Cas9 nickase variants, the Cas9 proteins used herein may also include other “Cas9 variants” having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, the disclosure also may utilize Cas9 fragments that retain their functionality and that are fragments of any herein disclosed Cas9 protein. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In various embodiments, the prime editors disclosed herein may comprise one of the Cas9 variants described as follows, or a Cas9 variant thereof having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 variants.

TABLE 4
Cas9 orthologs

| | | |
|---|---|---|
| Streptococcus | MDKKYSIGLD IGTNSVGWAV ITDEYKVPSK KFKVLGNTDR HSIKKNLIGA | (SEQ |
| pyogenes | LLFDSGETAE ATRLKRTARR RYTRRKNRIC YLQEIFSNEM AKVDDSFFHR | ID |
| AJN60024.1 | LEESFLVEED KKHERHPIFG NIVDEVAYHE KYPTIYHLRK KLVDSTDKAD | NO: |
| GI: 757015980 | LRLIYLALAH MIKFRGHFLI EGDLNPDNSD VDKLFIQLVQ TYNQLFEENP | 21) |
| WP_010922251.1 | INASGVDAKA ILSARLSKSR RLENLIAQLP GEKKNGLFGN LIALSLGLTP | |
| | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNFDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IEMARENQTT QKGQKNSRER MKRIEEGIKE LGSQILKEHP | |
| | VENTQLQNEK LYLYYLQNGR DMYVDQELDI NRLSDYDVDH IVPQSFLKDD | |
| | SIDNKVLTRS DKNRGKSDNV PSEEVVKKMK NYWRQLLNAK LITQRKFDNL | |
| | TKAERGGLSE LDKAGFIKRQ LVETRQITKH VAQILDSRMN TKYDENDKLI | |
| | REVKVITLKS KLVSDFRKDF QFYKVREINN YHHAHDAYLN AVVGTALIKK | |
| | YPKLESEFVY GDYKVYDVRK MIAKSEQEIG KATAKYFFYS NIMNFFKTEI | |
| | TLANGEIRKR PLIETNGETG EIVWDKGRDF ATVRKVLSMP QVNIVKKTEV | |
| | QTGGFSKESI LPKRNSDKLI ARKKDWDPKK YGGFDSPTVA YSVLVVAKVE | |
| | KGKSKKLKSV KELLGITIME RSSFEKNPID FLEAKGYKEV KKDLIIKLPK | |
| | YSLFELENGR KRMLASAGEL QKGNELALPS KYVNFLYLAS HYEKLKGSPE | |
| | DNEQKQLFVE QHKHYLDEII EQISEFSKRV ILADANLDKV LSAYNKHRDK | |
| | PIREQAENII HLFTLTNLGA PAAFKYFDTT IDRKRYTSTK EVLDATLIHQ | |
| | SITGLYETRI DLS | |
| AJN60021.1 | MKRNYILGLD IGITSVGYGI IDYETRDVID AGVRLFKEAN VENNEGRRSK | (SEQ |
| GI: 757015977 | RGARRLKRRR RHRIQRVKKL LFDYNLLTDH SELSGINPYE ARVKGLSQKL | ID |
| J7RUA5.1 | SEEEFSAALL HLAKRRGVHN VNEVEEDTGN ELSTKEQISR NSKALEEKYV | NO |
| WP_053019794.1 | AELQLERLKK DGEVRGSINR FKTSDYVKEA KQLLKVQKAY HQLDQSFIDT | 22) |
| Staphylococcuss | YIDLLETRRT YYEGPGEGSP FGWKDIKEWY EMLMGHCTYF PEELRSVKYA | |
| aureus | YNADLYNALN DLNNLVITRD ENEKLEYYEK FQIIENVFKQ KKKPTLKQIA | |
| | KEILVNEEDI KGYRVTSTGK PEFTNLKVYH DIKDITARKE IIENAELLDQ | |
| | IAKILTIYQS SEDIQEELTN LNSELTQEEI EQISNLKGYT GTHNLSLKAI | |
| | NLILDELWHT NDNQIAIFNR LKLVPKKVDL SQQKEIPTTL VDDFILSPVV | |
| | KRSFIQSIKV INAIIKKYGL PNDIIIELAR EKNSKDAQKM INEMQKRNRQ | |
| | TNERIEEIIR TTGKENAKYL IEKIKLHDMQ EGKCLYSLEA IPLEDLLNNP | |
| | FNYEVDHIIP RSVSFDNSFN NKVLVKQEEN SKKGNRTPFQ YLSSSDSKIS | |
| | YETFKKHILN LAKGKGRISK TKKEYLLEER DINRFSVQKD FINRNLVDTR | |
| | YATRGLMNLL RSYFRVNNLD VKVKSINGGF TSFLRRKWKF KKERNKGYKH | |
| | HAEDALIIAN ADFIFKEWKK LDKAKKVMEN QMFEEKQAES MPEIETEQEY | |
| | KEIFITPHQI KHIKDFKDYK YSHRVDKKPN RELINDTLYS TRKDDKGNTL | |
| | IVNNLNGLYD KDNDKLKKLI NKSPEKLLMY HHDPQTYQKL KLIMEQYGDE | |
| | KNPLYKYYEE TGNYLTKYSK KDNGPVIKKI KYYGNKLNAH LDITDDYPNS | |
| | RNKVVKLSLK PYRFDVYLDN GVYKFVTVKN LDVIKKENYY EVNSKCYEEA | |
| | KKLKKISNQA EFIASFYNND LIKINGELYR VIGVNNDLLN RIEVNMIDIT | |
| | YREYLENMND KRPPRIIKTI ASKTQSIKKY STDILGNLYE VKSKKHPQII | |
| | KKG | |
| AJN60008.1 | MARILAFDIG ISSIGWAFSE NDELKDCGVR IFTKVENPKT GESLALPRRL | (SEQ |
| GI: 757015964 | ARSARKRLAR RKARLNHLKH LIANEFKLNY EDYQSFDESL AKAYKGSLIS | ID |
| WP_002864485.1 | PYELRFRALN ELLSKQDFAR VILHIAKRRG YDDIKNSDDK EKGAILKAIK | NO: |
| Campylobacter | QNEEKLANYQ SVGEYLYKEY FQKFKENSKE FTNVRNKKES YERCIAQSFL | 23) |
| jejuni subsp. | KDELKLIFKK QREFGFSFSK KFEEEVLSVA FYKRALKDFS HLVGNCSFFT | |
| jejuni NCTC | DEKRAPKNSP LAFMFVALTR IINLLNNLKN TEGILYTKDD LNALLNEVLK | |
| 11168 = | NGTLTYKQTK KLLGLSDDYE FKGEKGTYFI EFKKYKEFIK ALGEHNLSQD | |
| ATCC 700819 | DLNEIAKDIT LIKDEIKLKK ALAKYDLNQN QIDSLSKLEF KDHLNISFKA | |
| | LKLVTPLMLE GKKYDEACNE LNLKVAINED KKDFLPAFNE TYYKDEVTNP | |
| | VVLRAIKEYR KVLNALLKKY GKVHKINIEL AREVGKNHSQ RAKIEKEQNE | |
| | NYKAKKDAEL ECEKLGLKIN SKNILKLRLF KEQKEFCAYS GEKIKISDLQ | |
| | DEKMLEIDHI YPYSRSFDDS YMNKVLVFTK QNQEKLNQTP FEAFGNDSAK | |
| | WQKIEVLAKN LPTKKQKRIL DKNYKDKEQK NFKDRNLNDT RYIARLVLNY | |
| | TKDYLDFLPL SDDENTKLND TQKGSKVHVE AKSGMLTSAL RHTWGFSAKD | |
| | RNNHLHHAID AVIIAYANNS IVKAFSDFKK EQESNSAELY AKKISELDYK | |
| | NKRKFFEPFS GFRQKVLDKI DEIFVSKPER KKPSGALHEE TFRKEEEFYQ | |
| | SYGGKEGVLK ALELGKIRKV NGKIVKNGDM FRVDIFKHKK TNKFYAVPIY | |
| | TMDFALKVLP NKAVARSKKG EIKDWILMDE NYEFCFSLYK DSLILIQTKD | |
| | MQEPEFVYYN AFTSSTVSLI VSKHDNKFET LSKNQKILFK NANEKEVIAK | |
| | SIGIQNLKVF EKYIVSALGE VTKAEFRQRE DFKK | |
| Streptococcus | MSDLVLGLDI GIGSVGVGIL NKVTGEIIHK NSRIFPAAQA ENNLVRRTNR | (SEQ |
| thermophilus | QGRRLARRKK HRRVRLNRLF EESGLITDFT KISINLNPYQ LRVKGLTDEL | ID |
| LMD-9 | SNEELFIALK NMVKHRGISY LDDASDDGNS SVGDYAQIVK ENSKQLETKT | NO: |
| AJN60026.1 | PGQIQLERYQ TYGQLRGDFT VEKDGKKHRL INVFPTSAYR SEALRILQTQ | 24) |
| GI: 757015982 | QEFNPQITDE FINRYLEILT GKRKYYHGPG NEKSRTDYGR YRTSGETLDN | |
| WP_011680957.1 | IFGILIGKCT FYPDEFRAAK ASYTAQEFNL LNDLNNLTVP TETKKLSKEQ | |
| | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARETNEDDEK KAIQKIQKAN KDEKDAAMLK AANQYNGKAE | |
| | LPHSVFHGHK QLATKIRLWH QQGERCLYTG KTISIHDLIN NSNQFEVDHI | |
| | LPLSITFDDS LANKVLVYAT ANQEKGQRTP YQALDSMDDA WSFRELKAFV | |
| | RESKTLSNKK KEYLLTEEDI SKFDVRKKFI ERNLVDTRYA SRVVLNALQE | |
| | HFRAHKIDTK VSVVRGQFTS QLRRHWGIEK TRDTYHHHAV DALIIAASSQ | |
| | LNLWKKQKNT LVSYSEDQLL DIETGELISD DEYKESVFKA PYQHFVDTLK | |
| | SKEFEDSILF SYQVDSKFNR KISDATIYAT RQAKVGKDKA DETYVLGKIK | |
| | DIYTQDGYDA FMKIYKKDKS KFLMYRHDPQ TFEKVIEPIL ENYPNKQINE | |
| | KGKEVPCNPF LKYKEEHGYI RKYSKKGNGP EIKSLKYYDS KLGNHIDITP | |
| | KDSNNKVVLQ SVSPWRADVY FNKTTGKYEI LGLKYADLQF EKGTGTYKIS | |
| | QEKYNDIKKK EGVDSDSEFK FTLYKNDLLL VKDTETKEQQ LFRFLSRTMP | |
| | KQKHYVELKP YDKQKFEGGE ALIKVLGNVA NSGQCKKGLG KSNISIYKVR | |
| | TDVLGNQHII KNEGDKPKLD F | |
| Parvibaculum | MERIFGFDIG TTSIGFSVID YSSTQSAGNI QRLGVRIFPE ARDPDGTPLN | (SEQ |
| lavamentivorans | QQRRQKRMMR RQLRRRRIRR KALNETLHEA GFLPAYGSAD WPVVMADEPY | ID |
| DS-1 | ELRRRGLEEG LSAYEFGRAI YHLAQHRHFK GRELEESDTP DPDVDDEKEA | NO: |
| AJN60020.1 | ANERAATLKA LKNEQTTLGA WLARRPPSDR KRGIHAHRNV VAEEFERLWE | 25) |
| GI: 757015976 | VQSKFHPALK SEEMRARISD TIFAQRPVFW RKNTLGECRF MPGEPLCPKG | |
| WP_011995013.1 | SWLSQQRRML EKLNNLAIAG GNARPLDAEE RDAILSKLQQ QASMSWPGVR | |
| | SALKALYKQR GEPGAEKSLK FNLELGGESK LLGNALEAKL ADMFGPDWPA | |
| | HPRKQEIRHA VHERLWAADY GETPDKKRVI ILSEKDRKAH REAAANSFVA | |
| | DFGITGEQAA QLQALKLPTG WEPYSIPALN LFLAELEKGE RFGALVNGPD | |
| | WEGWRRTNFP HRNQPTGEIL DKLPSPASKE ERERISQLRN PTVVRTQNEL | |
| | RKVVNNLIGL YGKPDRIRIE VGRDVGKSKR EREEIQSGIR RNEKQRKKAT | |
| | EDLIKNGIAN PSRDDVEKWI LWKEGQERCP YTGDQIGFNA LFREGRYEVE | |
| | HIWPRSRSFD NSPRNKTLCR KDVNIEKGNR MPFEAFGHDE DRWSAIQIRL | |
| | QGMVSAKGGT GMSPGKVKRF LAKTMPEDFA ARQLNDTRYA AKQILAQLKR | |
| | LWPDMGPEAP VKVEAVTGQV TAQLRKLWTL NNILADDGEK TRADHRHHAI | |
| | DALTVACTHP GMTNKLSRYW QLRDDPRAEK PALTPPWDTI RADAEKAVSE | |
| | IVVSHRVRKK VSGPLHKETT YGDTGTDIKT KSGTYRQFVT RKKIESLSKG | |
| | ELDEIRDPRI KEIVAAHVAG RGGDPKKAFP PYPCVSPGGP EIRKVRLTSK | |
| | QQLNLMAQTG NGYADLGSNH HIAIYRLPDG KADFEIVSLF DASRRLAQRN | |
| | PIVQRTRADG ASFVMSLAAG EAIMIPEGSK KGIWIVQGVW ASGQVVLERD | |
| | TDADHSTTTR PMPNPILKDD AKKVSIDPIG RVRPSND | |
| Corynebacterium | MKYHVGIDVG TFSVGLAAIE VDDAGMPIKT LSLVSHIHDS GLDPDEIKSA | (SEQ |
| diphtheriae | VTRLASSGIA RRTRRLYRRK RRRLQQLDKF IQRQGWPVIE LEDYSDPLYP | ID |
| NCTC 13129 | WKVRAELAAS YIADEKERGE KLSVALRHIA RHRGWRNPYA KVSSLYLPDG | NO: |
| AJN60012.1 | PSDAFKAIRE EIKRASGQPV PETATVGQMV TLCELGTLKL RGEGGVLSAR | 26) |
| GI: 757015968 | LQQSDYAREI QEICRMQEIG QELYRKIIDV VFAAESPKGS ASSRVGKDPL | |
| WP_010933968.1 | QPGKNRALKA SDAFQRYRIA ALIGNLRVRV DGEKRILSVE EKNLVFDHLV | |
| | NLTPKKEPEW VTIAEILGID RGQLIGTATM TDDGERAGAR PPTHDTNRSI | |
| | VNSRIAPLVD WWKTASALEQ HAMVKALSNA EVDDFDSPEG AKVQAFFADL | |
| | DDDVHAKLDS LHLPVGRAAY SEDTLVRLTR RMLSDGVDLY TARLQEFGIE | |
| | PSWTPPTPRI GEPVGNPAVD RVLKTVSRWL ESATKTWGAP ERVIIEHVRE | |
| | GFVTEKRARE MDGDMRRRAA RNAKLFQEMQ EKLNVQGKPS RADLWRYQSV | |
| | QRQNCQCAYC GSPITFSNSE MDHIVPRAGQ GSTNTRENLV AVCHRCNQSK | |
| | GNTPFAIWAK NTSIEGVSVK EAVERTRHWV TDTGMRSTDF KKFTKAVVER | |
| | FQRATMDEEI DARSMESVAW MANELRSRVA QHFASHGTTV RVYRGSLTAE | |
| | ARRASGISGK LKFFDGVGKS RLDRRHHAID AAVIAFTSDY VAETLAVRSN | |
| | LKQSQAHRQE APQWREFTGK DAEHRAAWRV WCQKMEKLSA LLTEDLRDDR | |
| | VVVMSNVRLR LGNGSAHKET IGKLSKVKLS SQLSVSDIDK ASSEALWCAL | |
| | TREPGFDPKE GLPANPERHI RVNGTHVYAG DNIGLFPVSA GSIALRGGYA | |
| | ELGSSFHHAR VYKITSGKKP AFAMLRVYTI DLLPYRNQDL FSVELKPQTM | |
| | SMRQAEKKLR DALATGNAEY LGWLVVDDEL VVDTSKIATD QVKAVEAELG | |
| | TIRRWRVDGF FSPSKLRLRP LQMSKEGIKK ESAPELSKII DRPGWLPAVN | |
| | KLFSDGNVTV VRRDSLGRVR LESTAHLPVT WKVQ | |
| Streptococcus | MTNGKILGLD IGIASVGVGI IEAKTGKVVH ANSRLFSAAN AENNAERRGF | (SEQ |
| pasteurianus | RGSRRLNRRK KHRVKRVRDL FEKYGIVTDF RNLNLNPYEL RVKGLTEQLK | ID |
| WP_013852048.1 | NEELFAALRT ISKRRGISYL DDAEDDSTGS TDYAKSIDEN RRLLKNKTPG | NO: |
| | QIQLERLEKY GQLRGNFTVY DENGEAHRLI NVFSTSDYEK EARKILETQA | 27) |
| | DYNKKITAEF IDDYVEILTQ KRKYYHGPGN EKSRTDYGRF RTDGTTLENI | |
| | FGILIGKCNF YPDEYRASKA SYTAQEYNFL NDLNNLKVST ETGKLSTEQK | |
| | ESLVEFAKNT ATLGPAKLLK EIAKILDCKV DEIKGYREDD KGKPDLHTFE | |
| | PYRKLKFNLE SINIDDLSRE VIDKLADILT LNTEREGIED AIKRNLPNQF | |
| | TEEQISEIIK VRKSQSTAFN KGWHSFSAKL MNELIPELYA TSDEQMTILT | |
| | RLEKFKVNKK SSKNTKTIDE KEVTDEIYNP VVAKSVRQTI KIINAAVKKY | |
| | GDFDKIVIEM PRDKNADDEK KFIDKRNKEN KKEKDDALKR AAYLYNSSDK | |
| | LPDEVFHGNK QLETKIRLWY QQGERCLYSG KPISIQELVH NSNNFEIDHI | |
| | LPLSLSFDDS LANKVLVYAW TNQEKGQKTP YQVIDSMDAA WSFREMKDYV | |
| | LKQKGLGKKK RDYLLTTENI DKIEVKKKFI ERNLVDTRYA SRVVLNSLQS | |
| | ALRELGKDTK VSVVRGQFTS QLRRKWKIDK SRETYHHHAV DALIIAASSQ | |
| | LKLWEKQDNP MFVDYGKNQV VDKQTGEILS VSDDEYKELV FQPPYQGFVN | |
| | TISSKGFEDE ILFSYQVDSK YNRKVSDATI YSTRKAKIGK DKKEETYVLG | |
| | KIKDIYSQNG FDTFIKKYNK DKTQFLMYQK DSLTWENVIE VILRDYPTTK | |
| | KSEDGKNDVK CNPFEEYRRE NGLICKYSKK GKGTPIKSLK YYDKKLGNCI | |
| | DITPEESRNK VILQSINPWR ADVYFNPETL KYELMGLKYS DLSFEKGTGN | |
| | YHISQEKYDA IKEKEGIGKK SEFKFTLYRN DLILIKDIAS GEQEIYRFLS | |
| | RTMPNVNHYV ELKPYDKEKF DNVQELVEAL GEADKVGRCI KGLNKPNISI | |
| | YKVRTDVLGN KYFVKKKGDK PKLDFKNNK K | |
| Neisseria | MAAFKPNPMN YILGLDIGIA SVGWAIVEID EEENPIRLID LGVRVFERAE | (SEQ |
| cinerea ATCC | VPKTGDSLAA ARRLARSVRR LTRRRAHRLL RARRLLKREG VLQAADFDEN | ID |
| 14685 | GLIKSLPNTP WQLRAAALDR KLTPLEWSAV LLHLIKHRGY LSQRKNEGET | NO: |
| AJN60019.1 | ADKELGALLK GVADNTHALQ TGDFRTPAEL ALNKFEKESG HIRNQRGDYS | 28) |
| GI: 757015975 | HTFNRKDLQA ELNLLFEKQK EFGNPHVSDG LKEGIETLLM TQRPALSGDA | |
| WP_003676410.1 | VQKMLGHCTF EPTEPKAAKN TYTAERFVWL TKLNNLRILE QGSERPLTDT | |
| | ERATLMDEPY RKSKLTYAQA RKLLDLDDTA FFKGLRYGKD NAEASTLMEM | |
| | KAYHAISRAL EKEGLKDKKS PLNLSPELQD EIGTAFSLFK TDEDITGRLK | |
| | DRVQPEILEA LLKHISFDKF VQISLKALRR IVPLMEQGNR YDEACTEIYG | |
| | DHYGKKNTEE KIYLPPIPAD EIRNPVVLRA LSQARKVING VVRRYGSPAR | |
| | IHIETAREVG KSFKDRKEIE KRQEENRKDR EKSAAKFREY FPNFVGEPKS | |
| | KDILKLRLYE QQHGKCLYSG KEINLGRLNE KGYVEIDHAL PFSRTWDDSF | |
| | NNKVLALGSE NQNKGNQTPY EYFNGKDNSR EWQEFKARVE TSRFPRSKKQ | |
| | RILLQKFDED GFKERNLNDT RYINRFLCQF VADHMLLTGK GKRRVFASNG | |
| | QITNLLRGFW GLRKVRAEND RHHALDAVVV ACSTIAMQQK ITRFVRYKEM | |
| | NAFDGKTIDK ETGEVLHQKA HFPQPWEFFA QEVMIRVFGK PDGKPEFEEA | |
| | DTPEKLRTLL AEKLSSRPEA VHKYVTPLFI SRAPNRKMSG QGHMETVKSA | |
| | KRLDEGISVL RVPLTQLKLK DLEKMVNRER EPKLYEALKA RLEAHKDDPA | |
| | KAFAEPFYKY DKAGNRTQQV KAVRVEQVQK TGVWVHNHNG IADNATIVRV | |
| | DVFEKGGKYY LVPIYSWQVA KGILPDRAVV QGKDEEDWTV MDDSFEFKFV | |
| | LYANDLIKLT AKKNEFLGYF VSLNRATGAI DIRTHDTDST KGKNGIFQSV | |
| | GVKTALSFQK YQIDELGKEI RPCRLKKRPP VR | |
| AJN60009.1 | MSDLVLGLDI GIGSVGVGIL NKVTGEIIHK NSRIFPAAQA ENNLVRRTNR | (SEQ |
| GI: 757015965 | QGRRLARRKK HRRVRLNRLF EESGLITDFT KISININPYQ LRVKGLTDEL | ID |
| St1Cas9 + SpCas9 | SNEELFIALK NMVKHRGISY LDDASDDGNS SVGDYAQIVK ENSKQLETKT | NO: |
| | PGQIQLERYQ TYGQLRGDFT VEKDGKKHRL INVFPTSAYR SEALRILQTQ | 29 |
| | QEFNPQITDE FINRYLEILT GKRKYYHGPG NEKSRTDYGR YRTSGETLDN | |
| | IFGILIGKCT FYPDEFRAAK ASYTAQEFNL LNDLNNLTVP TETKKLSKEQ | |
| | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARENQTTQKG QKNSRERMKR IEEGIKELGS QILKEHPVEN | |
| | TQLQNEKLYL YYLQNGRDMY VDQELDINRL SDYDVDHIVP QSFLKDDSID | |
| | NKVLTRSDKN RGKSDNVPSE EVVKKMKNYW RQLLNAKLIT QRKFDNLTKA | |
| | ERGGLSELDK AGFIKRQLVE TRQITKHVAQ ILDSRMNTKY DENDKLIREV | |
| | KVITLKSKLV SDFRKDFQFY KVREINNYHH AHDAYLNAVV GTALIKKYPK | |
| | LESEFVYGDY KVYDVRKMIA KSEQEIGKAT AKYFFYSNIM NFFKTEITLA | |
| | NGEIRKRPLI ETNGETGEIV WDKGRDFATV RKVLSMPQVN IVKKTEVQTG | |
| | GFSKESILPK RNSDKLIARK KDWDPKKYGG FDSPTVAYSV LVVAKVEKGK | |
| | SKKLKSVKEL LGITIMERSS FEKNPIDFLE AKGYKEVKKD LIIKLPKYSL | |
| | FELENGRKRM LASAGELQKG NELALPSKYV NFLYLASHYE KLKGSPEDNE | |
| | QKQLFVEQHK HYLDEIIEQI SEFSKRVILA DANLDKVLSA YNKHRDKPIR | |
| | EQAENIIHLF TLTNLGAPAA FKYFDTTIDR KRYTSTKEVL DATLIHQSIT | |
| | GLYETRIDLS QLGGD | |
| Campylobacter | MRILGFDIGI NSIGWAFVEN DELKDCGVRI FTKAENPKNK ESLALPRRNA | (SEQ |
| lari Cas9 | RSSRRRLKRR KARLIAIKRI LAKELKLNYK DYVAADGELP KAYEGSLASV | ID |
| BAK69486.1 | YELRYKALTQ NLETKDLARV ILHIAKHRGY MNKNEKKSND AKKGKILSAL | NO: |
| | KNNALKLENY QSVGEYFYKE FFQKYKKNTK NFIKIRNTKD NYNNCVLSSD | 30) |
| | LEKELKLILE KQKEFGYNYS EDFINEILKV AFFQRPLKDF SHLVGACTFF | |
| | EEEKRACKNS YSAWEFVALT KIINEIKSLE KISGEIVPTQ TINEVLNLIL | |
| | DKGSITYKKF RSCINLHESI SFKSLKYDKE NAENAKLIDF RKLVEFKKAL | |
| | GVHSLSRQEL DQISTHITLI KDNVKLKTVL EKYNLSNEQI NNLLEIEFND | |
| | YINLSFKALG MILPLMREGK RYDEACEIAN LKPKTVDEKK DFLPAFCDSI | |
| | FAHELSNPVV NRAISEYRKV LNALLKKYGK VHKIHLELAR DVGLSKKARE | |
| | KIEKEQKENQ AVNAWALKEC ENIGLKASAK NILKLKLWKE QKEICIYSGN | |
| | KISIEHLKDE KALEVDHIYP YSRSFDDSFI NKVLVFTKEN QEKLNKTPFE | |
| | AFGKNIEKWS KIQTLAQNLP YKKKNKILDE NFKDKQQEDF ISRNLNDTRY | |
| | IATLIAKYTK EYLNFLLLSE NENANLKSGE KGSKIHVQTI SGMLTSVLRH | |
| | TWGFDKKDRN NHLHHALDAI IVAYSTNSII KAFSDFRKNQ ELLKARFYAK | |
| | ELTSDNYKHQ VKFFEPFKSF REKILSKIDE IFVSKPPRKR ARRALHKDTF | |
| | HSENKIIDKC SYNSKEGLQI ALSCGRVRKI GTKYVENDTI VRVDIFKKQN | |
| | KFYAIPIYAM DFALGILPNK IVITGKDKNN NPKQWQTIDE SYEFCFSLYK | |
| | NDLILLQKKN MQEPEFAYYN DFSISTSSIC VEKHDNKFEN LTSNQKLLFS | |
| | NAKEGSVKVE SLGIQNLKVF EKYIITPLGD KIKADFQPRE NISLKTSKKY | |
| | GLR | |
| AJN60010.1 | MDKKYSIGLD IGTNSVGWAV ITDEYKVPSK KFKVLGNTDR HSIKKNLIGA | (SEQ |
| GI: 757015966 | LLFDSGETAE ATRLKRTARR RYTRRKNRIC YLQEIFSNEM AKVDDSFFHR | ID |
| SpCas9 + St1Cas9 | LEESFLVEED KKHERHPIFG NIVDEVAYHE KYPTIYHLRK KLVDSTDKAD | NO: |
| | LRLIYLALAH MIKFRGHFLI EGDLNPDNSD VDKLFIQLVQ TYNQLFEENP | 31) |
| | INASGVDAKA ILSARLSKSR RLENLIAQLP GEKKNGLFGN LIALSLGLTP | |
| | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNFDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IEMARETNED DEKKAIQKIQ KANKDEKDAA MLKAANQYNG | |
| | KAELPHSVFH GHKQLATKIR LWHQQGERCL YTGKTISIHD LINNSNQFEV | |
| | DHILPLSITF DDSLANKVLV YATANQEKGQ RTPYQALDSM DDAWSFRELK | |
| | AFVRESKTLS NKKKEYLLTE EDISKFDVRK KFIERNLVDT RYASRVVLNA | |
| | LQEHFRAHKI DTKVSVVRGQ FTSQLRRHWG IEKTRDTYHH HAVDALIIAA | |
| | SSQLNLWKKQ KNTLVSYSED QLLDIETGEL ISDDEYKESV FKAPYQHFVD | |
| | TLKSKEFEDS ILFSYQVDSK FNRKISDATI YATRQAKVGK DKADETYVLG | |
| | KIKDIYTQDG YDAFMKIYKK DKSKFLMYRH DPQTFEKVIE PILENYPNKQ | |
| | INEKGKEVPC NPFLKYKEEH GYIRKYSKKG NGPEIKSLKY YDSKLGNHID | |
| | ITPKDSNNKV VLQSVSPWRA DVYFNKTTGK YEILGLKYAD LQFEKGTGTY | |
| | KISQEKYNDI KKKEGVDSDS EFKFTLYKND LLLVKDTETK EQQLFRFLSR | |
| | TMPKQKHYVE LKPYDKQKFE GGEALIKVLG NVANSGQCKK GLGKSNISIY | |
| | KVRTDVLGNQ HIIKNEGDKP KLDF | |
| SpCas9 | MDKKYSIGLD IGTNSVGWAV ITDEYKVPSK KFKVLGNTDR HSIKKNLIGA | (SEQ |
| inactive | LLFDSGETAE ATRLKRTARR RYTRRKNRIC YLQEIFSNEM AKVDDSFFHR | ID |
| AJN60011.1 | LEESFLVEED KKHERHPIFG NIVDEVAYHE KYPTIYHLRK KLVDSTDKAD | NO: |
| GI: 757015967 | LRLIYLALAH MIKFRGHFLI EGDLNPDNSD VDKLFIQLVQ TYNQLFEENP | 32) |
| | INASGVDAKA ILSARLSKSR RLENLIAQLP GEKKNGLFGN LIALSLGLTP | |
| | NFKSNFDLAE DAKLQLSKDT YDDDLDNLLA QIGDQYADLF LAAKNLSDAI | |
| | LLSDILRVNT EITKAPLSAS MIKRYDEHHQ DLTLLKALVR QQLPEKYKEI | |
| | FFDQSKNGYA GYIDGGASQE EFYKFIKPIL EKMDGTEELL VKLNREDLLR | |
| | KQRTFDNGSI PHQIHLGELH AILRRQEDFY PFLKDNREKI EKILTFRIPY | |
| | YVGPLARGNS RFAWMTRKSE ETITPWNFEE VVDKGASAQS FIERMTNFDK | |
| | NLPNEKVLPK HSLLYEYFTV YNELTKVKYV TEGMRKPAFL SGEQKKAIVD | |
| | LLFKTNRKVT VKQLKEDYFK KIECFDSVEI SGVEDRFNAS LGTYHDLLKI | |
| | IKDKDFLDNE ENEDILEDIV LTLTLFEDRE MIEERLKTYA HLFDDKVMKQ | |
| | LKRRRYTGWG RLSRKLINGI RDKQSGKTIL DFLKSDGFAN RNFMQLIHDD | |
| | SLTFKEDIQK AQVSGQGDSL HEHIANLAGS PAIKKGILQT VKVVDELVKV | |
| | MGRHKPENIV IAMARENQTT QKGQKNSRER MKRIEEGIKE LGSQILKEHP | |
| | VENTQLQNEK LYLYYLQNGR DMYVDQELDI NRLSDYDVDA IVPQSFLKDD | |
| | SIDAKVLTRS DKARGKSDNV PSEEVVKKMK NYWRQLLNAK LITQRKFDNL | |
| | TKAERGGLSE LDKAGFIKRQ LVETRQITKH VAQILDSRMN TKYDENDKLI | |
| | REVKVITLKS KLVSDFRKDF QFYKVREINN YHHAHAAYLN AVVGTALIKK | |
| | YPKLESEFVY GDYKVYDVRK MIAKSEQEIG KATAKYFFYS NIMNFFKTEI | |
| | TLANGEIRKR PLIETNGETG EIVWDKGRDF ATVRKVLSMP QVNIVKKTEV | |
| | QTGGFSKESI LPKRNSDKLI ARKKDWDPKK YGGFDSPTVA YSVLVVAKVE | |
| | KGKSKKLKSV KELLGITIME RSSFEKNPID FLEAKGYKEV KKDLIIKLPK | |
| | YSLFELENGR KRMLASAGEL QKGNELALPS KYVNFLYLAS HYEKLKGSPE | |
| | DNEQKQLFVE QHKHYLDEII EQISEFSKRV ILADANLDKV LSAYNKHRDK | |
| | PIREQAENII HLFTLINLGA PAAFKYFDTT IDRKRYTSTK EVLDATLIHQ | |
| | SITGLYETRI DLSQLGGD | |
| AJN60013.1 | MTQSERRFSC SIGIDMGAKY TGVFYALFDR EELPTNLNSK AMTLVMPETG | (SEQ |
| GI: 757015969 | PRYVQAQRTA VRHRLRGQKR YTLARKLAFL VVDDMIKKQE KRLTDEEWKR | ID |
| WP_005430658.1 | GREALSGLLK RRGYSRPNAD GEDLTPLENV RADVFAAHPA FSTYFSEVRS | NO: |
| Sutterella | LAEQWEEFTA NISNVEKFLG DPNIPADKEF IEFAVAEGLI DKTEKKAYQS | 33) |
| wadsworthensis | ALSTLRANAN VLTGLRQMGH KPRSEYFKAI EADLKKDSRL AKINEAFGGA | |
| 3_1_45B | ERLARLLGNL SNLQLRAERW YFNAPDIMKD RGWEPDRFKK TLVRAFKFFH | |
| | PAKDQNKQHL ELIKQIENSE DIIETLCTLD PNRTIPPYED QNNRRPPLDQ | |
| | TLLLSPEKLT RQYGEIWKTW SARLTSAEPT LAPAAEILER STDRKSRVAV | |
| | NGHEPLPTLA YQLSYALQRA FDRSKALDPY ALRALAAGSK SNKLTSARTA | |
| | LENCIGGQNV KTFLDCARRY YREADDAKVG LWFDNADGLL ERSDLHPPMK | |
| | KKILPLLVAN ILQTDETTGQ KFLDEIWRKQ IKGRETVASR CARIETVRKS | |
| | FGGGFNIAYN TAQYREVNKL PRNAQDKELL TIRDRVAETA DFIAANLGLS | |
| | DEQKRKFANP FSLAQFYTLI ETEVSGFSAT TLAVHLENAW RMTIKDAVIN | |
| | GETVRAAQCS RLPAETARPF DGLVRRLVDR QAWEIAKRVS TDIQSKVDFS | |
| | NGIVDVSIFV EENKFEFSAS VADLKKNKRV KDKMLSEAEK LETRWLIKNE | |
| | RIKKASRGTC PYTGDRLAEG GEIDHILPRS LIKDARGIVF NAEPNLIYAS | |
| | SRGNQLKKNQ RYSLSDLKAN YRNEIFKTSN IAAITAEIED VVTKLQQTHR | |
| | LKFFDLLNEH EQDCVRHALF LDDGSEARDA VLELLATQRR TRVNGTQIWM | |
| | IKNLANKIRE ELQNWCKTTN NRLHFQAAAT NVSDAKNLRL KLAQNQPDFE | |
| | KPDIQPIASH SIDALCSFAV GSADAERDQN GFDYLDGKTV LGLYPQSCEV | |
| | IHLQAKPQEE KSHFDSVAIF KEGIYAEQFL PIFTLNEKIW IGYETLNAKG | |
| | ERCGAIEVSG KQPKELLEML APFFNKPVGD LSAHATYRIL KKPAYEFLAK | |
| | AALQPLSAEE KRLAALLDAL RYCTSRKSLM SLFMAANGKS LKKREDVLKP | |
| | KLFQLKVELK GEKSFKLNGS LTLPVKQDWL RICDSPELAD AFGKPCSADE | |
| | LTSKLARIWK RPVMRDLAHA PVRREFSLPA IDNPSGGFRI RRTNLFGNEL | |
| | YQVHAINAKK YRGFASAGSN VDWSKGILFN ELQHENLTEC GGRFITSADV | |
| | TPMSEWRKVV AEDNLSIWIA PGTEGRRYVR VETTFIQASH WFEQSVENWA | |
| | ITSPLSLPAS FKVDKPAEFQ KAVGTELSEL LGQPRSEIFI ENVGNAKHIR | |
| | FWYIVVSSNK KMNESYNNVS KS | |
| AJN60014.1 | MESSQILSPI GIDLGGKFTG VCLSHLEAFA ELPNHANTKY SVILIDHNNF | (SEQ |
| GI: 757015970 | QLSQAQRRAT RHRVRNKKRN QFVKRVALQL FQHILSRDLN AKEETALCHY | ID |
| WP_011212792.1 | LNNRGYTYVD TDLDEYIKDE TTINLLKELL PSESEHNFID WFLQKMQSSE | NO: |
| Legionella | FRKILVSKVE EKKDDKELKN AVKNIKNFIT GFEKNSVEGH RHRKVYFENI | 34) |
| pneumophila | KSDITKDNQL DSIKKKIPSV CLSNLLGHLS NLQWKNLHRY LAKNPKQFDE | |
| str. Paris | QTFGNEFLRM LKNFRHLKGS QESLAVRNLI QQLEQSQDYI SILEKTPPEI | |
| | TIPPYEARTN TGMEKDQSLL LNPEKLNNLY PNWRNLIPGI IDAHPFLEKD | |
| | LEHTKLRDRK RIISPSKQDE KRDSYILQRY LDLNKKIDKF KIKKQLSFLG | |
| | QGKQLPANLI ETQKEMETHF NSSLVSVLIQ IASAYNKERE DAAQGIWFDN | |
| | AFSLCELSNI NPPRKQKILP LLVGAILSED FINNKDKWAK FKIFWNTHKI | |
| | GRTSLKSKCK EIEEARKNSG NAFKIDYEEA LNHPEHSNNK ALIKIIQTIP | |
| | DIIQAIQSHL GHNDSQALIY HNPFSLSQLY TILETKRDGF HKNCVAVTCE | |
| | NYWRSQKTEI DPEISYASRL PADSVRPFDG VLARMMQRLA YEIAMAKWEQ | |
| | IKHIPDNSSL LIPIYLEQNR FEFEESFKKI KGSSSDKTLE QAIEKQNIQW | |
| | EEKFQRIINA SMNICPYKGA SIGGQGEIDH IYPRSLSKKH FGVIFNSEVN | |
| | LIYCSSQGNR EKKEEHYLLE HLSPLYLKHQ FGTDNVSDIK NFISQNVANI | |
| | KKYISFHLLT PEQQKAARHA LFLDYDDEAF KTITKFLMSQ QKARVNGTQK | |
| | FLGKQIMEFL STLADSKQLQ LEFSIKQITA EEVHDHRELL SKQEPKLVKS | |
| | RQQSFPSHAI DATLTMSIGL KEFPQFSQEL DNSWFINHLM PDEVHLNPVR | |
| | SKEKYNKPNI SSTPLFKDSL YAERFIPVWV KGETFAIGFS EKDLFEIKPS | |
| | NKEKLFTLLK TYSTKNPGES LQELQAKSKA KWLYFPINKT LALEFLHHYF | |
| | HKEIVTPDDT TVCHFINSLR YYTKKESITV KILKEPMPVL SVKFESSKKN | |
| | VLGSFKHTIA LPATKDWERL FNHPNFLALK ANPAPNPKEF NEFIRKYFLS | |
| | DNNPNSDIPN NGHNIKPQKH KAVRKVFSLP VIPGNAGTMM RIRRKDNKGQ | |
| | PLYQLQTIDD TPSMGIQINE DRLVKQEVLM DAYKTRNLST IDGINNSEGQ | |
| | AYATFDNWLT LPVSTFKPEI IKLEMKPHSK TRRYIRITQS LADFIKTIDE | |
| | ALMIKPSDSI DDPLNMPNEI VCKNKLFGNE LKPRDGKMKI VSTGKIVTYE | |
| | FESDSTPQWI QTLYVTQLKK QP | |
| AJN60015.1 | MKKEIKDYFL GLDVGTGSVG WAVTDTDYKL LKANRKDLWG MRCFETAETA | (SEQ |
| GI: 757015971 | EVRRLHRGAR RRIERRKKRI KLLQELFSQE IAKTDEGFFQ RMKESPFYAE | ID |
| WP_002681289.1 | DKTILQENTL FNDKDFADKT YHKAYPTINH LIKAWIENKV KPDPRLLYLA | NO: |
| Treponema | CHNIIKKRGH FLFEGDFDSE NQFDTSIQAL FEYLREDMEV DIDADSQKVK | 35) |
| denticola | EILKDSSLKN SEKQSRLNKI LGLKPSDKQK KAITNLISGN KINFADLYDN | |
| ATCC 35405 | PDLKDAEKNS ISFSKDDFDA LSDDLASILG DSFELLLKAK AVYNCSVLSK | |
| | VIGDEQYLSF AKVKIYEKHK TDLTKLKNVI KKHFPKDYKK VFGYNKNEKN | |
| | NNNYSGYVGV CKTKSKKLII NNSVNQEDFY KFLKTILSAK SEIKEVNDIL | |
| | TEIETGTFLP KQISKSNAEI PYQLRKMELE KILSNAEKHF SFLKQKDEKG | |
| | LSHSEKIIML LTFKIPYYIG PINDNHKKFF PDRCWVVKKE KSPSGKTTPW | |
| | NFFDHIDKEK TAEAFITSRT NFCTYLVGES VLPKSSLLYS EYTVLNEINN | |
| | LQIIIDGKNI CDIKLKQKIY EDLFKKYKKI TQKQISTFIK HEGICNKTDE | |
| | VIILGIDKEC TSSLKSYIEL KNIFGKQVDE ISTKNMLEEI IRWATIYDEG | |
| | EGKTILKTKI KAEYGKYCSD EQIKKILNLK FSGWGRLSRK FLETVTSEMP | |
| | GFSEPVNIIT AMRETQNNLM ELLSSEFTFT ENIKKINSGF EDAEKQFSYD | |
| | GLVKPLFLSP SVKKMLWQTL KLVKEISHIT QAPPKKIFIE MAKGAELEPA | |
| | RTKTRLKILQ DLYNNCKNDA DAFSSEIKDL SGKIENEDNL RLRSDKLYLY | |
| | YTQLGKCMYC GKPIEIGHVF DTSNYDIDHI YPQSKIKDDS ISNRVLVCSS | |
| | CNKNKEDKYP LKSEIQSKQR GFWNFLQRNN FISLEKLNRL TRATPISDDE | |
| | TAKFIARQLV ETRQATKVAA KVLEKMFPET KIVYSKAETV SMFRNKFDIV | |
| | KCREINDFHH AHDAYLNIVV GNVYNTKFTN NPWNFIKEKR DNPKIADTYN | |
| | YYKVFDYDVK RNNITAWEKG KTIITVKDML KRNTPIYTRQ AACKKGELFN | |
| | QTIMKKGLGQ HPLKKEGPFS NISKYGGYNK VSAAYYTLIE YEEKGNKIRS | |
| | LETIPLYLVK DIQKDQDVLK SYLTDLLGKK EFKILVPKIK INSLLKINGF | |
| | PCHITGKIND SFLLRPAVQF CCSNNEVLYF KKIIRFSEIR SQREKIGKTI | |
| | SPYEDLSFRS YIKENLWKKT KNDEIGEKEF YDLLQKKNLE IYDMLLTKHK | |
| | DTIYKKRPNS ATIDILVKGK EKFKSLIIEN QFEVILEILK LFSATRNVSD | |
| | LQHIGGSKYS GVAKIGNKIS SLDNCILIYQ SITGIFEKRI DLLKV | |
| AJN60016.1 | MTKEYYLGLD VGTNSVGWAV TDSQYNLCKF KKKDMWGIRL FESANTAKDR | (SEQ |
| GI: 757015972 | RLQRGNRRRL ERKKQRIDLL QEIFSPEICK IDPTFFIRLN ESRLHLEDKS | ID |
| EFE28295.1 | NDFKYPLFIE KDYSDIEYYK EFPTIFHLRK HLIESEEKQD IRLIYLALHN | NO: |
| Filifactor | IIKTRGHFLI DGDLQSAKQL RPILDTFLLS LQEEQNLSVS LSENQKDEYE | 36) |
| alocis ATCC | EILKNRSIAK SEKVKKLKNL FEISDELEKE EKKAQSAVIE NFCKFIVGNK | |
| 35896 | GDVCKFLRVS KEELEIDSFS FSEGKYEDDI VKNLEEKVPE KVYLFEQMKA | |
| | MYDWNILVDI LETEEYISFA KVKQYEKHKT NLRLLRDIIL KYCTKDEYNR | |
| | MFNDEKEAGS YTAYVGKLKK NNKKYWIEKK RNPEEFYKSL GKLLDKIEPL | |
| | KEDLEVLTMM IEECKNHTLL PIQKNKDNGV IPHQVHEVEL KKILENAKKY | |
| | YSFLTETDKD GYSVVQKIES IFRFRIPYYV GPLSTRHQEK GSNVWMVRKP | |
| | GREDRIYPWN MEEIIDFEKS NENFITRMTN KCTYLIGEDV LPKHSLLYSK | |
| | YMVLNELNNV KVRGKKLPTS LKQKVFEDLF ENKSKVTGKN LLEYLQIQDK | |
| | DIQIDDLSGF DKDFKTSLKS YLDFKKQIFG EEIEKESIQN MIEDIIKWIT | |
| | IYGNDKEMLK RVIRANYSNQ LTEEQMKKIT GFQYSGWGNF SKMFLKGISG | |
| | SDVSTGETFD IITAMWETDN NLMQILSKKF TFMDNVEDFN SGKVGKIDKI | |
| | TYDSTVKEMF LSPENKRAVW QTIQVAEEIK KVMGCEPKKI FIEMARGGEK | |
| | VKKRTKSRKA QLLELYAACE EDCRELIKEI EDRDERDENS MKLFLYYTQF | |
| | GKCMYSGDDI DINELIRGNS KWDRDHIYPQ SKIKDDSIDN LVLVNKTYNA | |
| | KKSNELLSED IQKKMHSFWL SLLNKKLITK SKYDRLTRKG DFTDEELSGF | |
| | IARQLVETRQ STKAIADIFK QIYSSEVVYV KSSLVSDFRK KPLNYLKSRR | |
| | VNDYHHAKDA YLNIVVGNVY NKKFTSNPIQ WMKKNRDTNY SLNKVFEHDV | |
| | VINGEVIWEK CTYHEDTNTY DGGTLDRIRK IVERDNILYT EYAYCEKGEL | |
| | FNATIQNKNG NSTVSLKKGL DVKKYGGYFS ANTSYFSLIE FEDKKGDRAR | |
| | HIIGVPIYIA NMLEHSPSAF LEYCEQKGYQ NVRILVEKIK KNSLLIINGY | |
| | PLRIRGENEV DTSFKRAIQL KLDQKNYELV RNIEKFLEKY VEKKGNYPID | |
| | ENRDHITHEK MNQLYEVLLS KMKKFNKKGM ADPSDRIEKS KPKFIKLEDL | |
| | IDKINVINKM LNLLRCDNDT KADLSLIELP KNAGSFVVKK NTIGKSKIIL | |
| | VNQSVTGLYE NRREI | |
| AJN60017.1 | MGRKPYILSL DIGTGSVGYA CMDKGFNVLK YHDKDALGVY LFDGALTAQE | (SEQ |
| GI: 757015973 | RRQFRTSRRR KNRRIKRLGL LQELLAPLVQ NPNFYQFQRQ FAWKNDNMDF | ID |
| WP_014613259.1 | KNKSLSEVLS FLGYESKKYP TIYHLQEALL LKDEKFDPEL IYMALYHLVK | NO: |
| Staphylococcus | YRGHFLFDHL KIENLTNNDN MHDFVELIET YENLNNIKLN LDYEKTKVIY | 37) |
| pseudintermedius | EILKDNEMTK NDRAKRVKNM EKKLEQFSIM LLGLKFNEGK LFNHADNAEE | |
| ED99 | LKGANQSHTF ADNYEENLTP FLTVEQSEFI ERANKIYLSL TLQDILKGKK | |
| | SMAMSKVAAY DKFRNELKQV KDIVYKADST RTQFKKIFVS SKKSLKQYDA | |
| | TPNDQTFSSL CLFDQYLIRP KKQYSLLIKE LKKIIPQDSE LYFEAENDTL | |
| | LKVLNTTDNA SIPMQINLYE AETILRNQQK YHAEITDEMI EKVLSLIQFR | |
| | IPYYVGPLVN DHTASKFGWM ERKSNESIKP WNFDEVVDRS KSATQFIRRM | |
| | TNKCSYLINE DVLPKNSLLY QEMEVLNELN ATQIRLQTDP KNRKYRMMPQ | |
| | IKLFAVEHIF KKYKTVSHSK FLEIMLNSNH RENFMNHGEK LSIFGTQDDK | |
| | KFASKLSSYQ DMTKIFGDIE GKRAQIEEII QWITIFEDKK ILVQKLKECY | |
| | PELTSKQINQ LKKLNYSGWG RLSEKLLTHA YQGHSIIELL RHSDENFMEI | |
| | LTNDVYGFQN FIKEENQVQS NKIQHQDIAN LTTSPALKKG IWSTIKLVRE | |
| | LTSIFGEPEK IIMEFATEDQ QKGKKQKSRK QLWDDNIKKN KLKSVDEYKY | |
| | IIDVANKLNN EQLQQEKLWL YLSQNGKCMY SGQSIDLDAL LSPNATKHYE | |
| | VDHIFPRSFI KDDSIDNKVL VIKKMNQTKG DQVPLQFIQQ PYERIAYWKS | |
| | LNKAGLISDS KLHKLMKPEF TAMDKEGFIQ RQLVETRQIS VHVRDFLKEE | |
| | YPNTKVIPMK AKMVSEFRKK FDIPKIRQMN DAHHAIDAYL NGVVYHGAQL | |
| | AYPNVDLFDF NFKWEKVREK WKALGEFNTK QKSRELFFFK KLEKMEVSQG | |
| | ERLISKIKLD MNHFKINYSR KLANIPQQFY NQTAVSPKTA ELKYESNKSN | |
| | EVVYKGLTPY QTYVVAIKSV NKKGKEKMEY QMIDHYVFDF YKFQNGNEKE | |
| | LALYLAQREN KDEVLDAQIV YSLNKGDLLY INNHPCYFVS RKEVINAKQF | |
| | ELTVEQQLSL YNVMNNKETN VEKLLIEYDF IAEKVINEYH HYLNSKLKEK | |
| | RVRTFFSESN QTHEDFIKAL DELFKVVTAS ATRSDKIGSR KNSMTHRAFL | |
| | GKGKDVKIAY TSISGLKTTK PKSLFKLAES RNEL | |
| AJN60018.1 | MTKIKDDYIV GLDIGTDSCG WVAMNSNNDI LKLQGKTAIG SRLFEGGKSA | (SEQ |
| GI: 757015974 | AERRLFRTTH RRIKRRRWRL KLLEEFFDPY MAEVDPYFFA RLKESGLSPL | ID |
| WP_014567561.1 | DKRKTVSSIV FPTSAEDKKF YDDYPTIYHL RYKLMTEDEK FDLREVYLAI | NO: |
| Lactobacillus | HHIIKYRGNF LYNTSVKDFK ASKIDVKSSI EKLNELYENL GLDLNVEFNI | 38) |
| johnsonii DPC | SNTAEIEKVL KDKQIFKRDK VKKIAELFAI KTDNKEQSKR IKDISKQVAN | |
| 6026 | AVLGYKTRFD TIALKEISKD ELSDWNFKLS DIDADSKFEA LMGNLDENEQ | |
| | AILLTIKELF NEVTLNGIVE DGNTLSESMI NKYNDHRDDL KLLKEVIENH | |
| | IDRKKAKELA LAYDLYVNNR HGQLLQAKKK LGKIKPRSKE DFYKVVNKNL | |
| | DDSRASKEIK KKIELDSFMP KQRTNANGVI PYQLQQLELD KIIENQSKYY | |
| | PFLKEINPVS SHLKEAPYKL DELIRFRVPY YVGPLISPNE STKDIQTKKN | |
| | QNFAWMIRKE EGRITPWNFD QKVDRIESAN KFIKRMTTKD TYLFGEDVLP | |
| | ANSLLYQKFT VLNELNNIRI NGKRISVDLK QEIYENLFKK HTTVTVKKLE | |
| | NYLKENHNLV KVEIKGLADE KKFNSGLTTY NRFKNLNIFD NQIDDLKYRN | |
| | DFEKIIEWST IFEDKSIYKE KLRSIDWLNE KQINALSNIR LQGWGRLSKK | |
| | LLAQLHDHNG QTIIEQLWDS QNNFMQIVTQ ADFKDAIAKA NQNLLVATSV | |
| | EDILNNAYTS PANKKAIRQV IKVVDDIVKA ASGKVPKQIA IEFTRDADEN | |
| | PKRSQTRGSK LQKVYKDLST ELASKTIAEE LNEAIKDKKL VQDKYYLYFM | |
| | QLGRDAYTGE PINIDEIQKY DIDHILPQSF IKDDALDNRV LVSRAVNNGK | |
| | SDNVPVKLFG NEMAANLGMT IRKMWEEWKN IGLISKTKYN NLLTDPDHIN | |
| | KYKSAGFIRR QLVETSQIIK LVSTILQSRY PNTEIITVKA KYNHYLREKF | |
| | DLYKSREVND YHHAIDAYLS AICGNLLYQN YPNLRPFFVY GQYKKFSSDP | |
| | DKEKAIFNKT RKFSFISQLL KNKSENSKEI AKKLKRAYQF KYMLVSRETE | |
| | TRDQEMFKMT VYPRFSHDTV KAPRNLIPKK MGMSPDIYGG YTNNSDAYMV | |
| | IVRIDKKKGT EYKILGIPTR ELVNLKKAEK EDHYKSYLKE ILTPRILYNK | |
| | NGKRDKKITS FEIVKSKIPY KQVIQDGDKK FMLGSSTYVY NAKQLTLSTE | |
| | SMKAITNNFD KDSDENDALI KAYDEILDKV DKYLPLFDIN KFREKLHSGR | |
| | EKFIKLSLED KKDTILKVLE GLHDNAVMTK IPTIGLSTPL GFMQFPNGVI | |
| | LSENAKLIYQ SPTGLFKKSV KISDL | |
| Mycoplasma | MNNSIKSKPE VTIGLDLGVG SVGWAIVDNE TNIIHHLGSR LFSQAKTAED | (SEQ |
| gallisepticum | RRSFRGVRRL IRRRKYKLKR FVNLIWKYNS YFGFKNKEDI LNNYQEQQKL | ID |
| str. F | HNTVLNLKSE ALNAKIDPKA LSWILHDYLK NRGHFYEDNR DFNVYPTKEL | NO: |
| AJN60022.1 | AKYFDKYGYY KGIIDSKEDN DNKLEEELTK YKFSNKHWLE EVKKVLSNQT | 39) |
| GI: 757015978 | GLPEKFKEEY ESLFSYVRNY SEGPGSINSV SPYGIYHLDE KEGKVVQKYN | |
| WP_014574789.1 | NIWDKTIGKC NIFPDEYRAP KNSPIAMIFN EINELSTIRS YSIYLTGWFI | |
| | NQEFKKAYLN KLLDLLIKTN GEKPIDARQF KKLREETIAE SIGKETLKDV | |
| | ENEEKLEKED HKWKLKGLKL NTNGKIQYND LSSLAKFVHK LKQHLKLDFL | |
| | LEDQYATLDK INFLQSLFVY LGKHLRYSNR VDSANLKEFS DSNKLFERIL | |
| | QKQKDGLFKL FEQTDKDDEK ILAQTHSLST KAMLLAITRM TNLDNDEDNQ | |
| | KNNDKGWNFE AIKNFDQKFI DITKKNNNLS LKQNKRYLDD RFINDAILSP | |
| | GVKRILREAT KVFNAILKQF SEEYDVTKVV IELARELSEE KELENTKNYK | |
| | KLIKKNGDKI SEGLKALGIS EDEIKDILKS PTKSYKFLLW LQQDHIDPYS | |
| | LKEIAFDDIF TKTEKFEIDH IIPYSISFDD SSSNKLLVLA ESNQAKSNQT | |
| | PYEFISSGNA GIKWEDYEAY CRKFKDGDSS LLDSTQRSKK FAKMMKTDTS | |
| | SKYDIGFLAR NLNDTRYATI VFRDALEDYA NNHLVEDKPM FKVVCINGSV | |
| | TSFLRKNFDD SSYAKKDRDK NIHHAVDASI ISIFSNETKT LFNQLTQFAD | |
| | YKLFKNTDGS WKKIDPKTGV VTEVTDENWK QIRVRNQVSE IAKVIEKYIQ | |
| | DSNIERKARY SRKIENKTNI SLFNDTVYSA KKVGYEDQIK RKNLKTLDIH | |
| | ESAKENKNSK VKRQFVYRKL VNVSLLNNDK LADLFAEKED ILMYRANPWV | |
| | INLAEQIFNE YTENKKIKSQ NVFEKYMLDL TKEFPEKFSE FLVKSMLRNK | |
| | TAIIYDDKKN IVHRIKRLKM LSSELKENKL SNVIIRSKNQ SGTKLSYQDT | |
| | INSLALMIMR SIDPTAKKQY IRVPLNTLNL HLGDHDFDLH NMDAYLKKPK | |
| | FVKYLKANEI GDEYKPWRVL TSGTLLIHKK DKKLMYISSF QNLNDVIEIK | |
| | NLIETEYKEN DDSDSKKKKK ANRFLMTLST ILNDYILLDA KDNFDILGLS | |
| | KNRIDEILNS KLGLDKIVK | |
| AJN60023.1 | MRILGFDIGI NSIGWAFVEN DELKDCGVRI FTKAENPKNK ESLALPRRNA | (SEQ |
| GI: 757015979 | RSSRRRLKRR KARLIAIKRI LAKELKLNYK DYVAADGELP KAYEGSLASV | ID |
| | YELRYKALTQ NLETKDLARV ILHIAKHRGY MNKNEKKSND AKKGKILSAL | NO: |
| | KNNALKLENY QSVGEYFYKE FFQKYKKNTK NFIKIRNTKD NYNNCVLSSD | 30) |
| | LEKELKLILE KQKEFGYNYS EDFINEILKV AFFQRPLKDF SHLVGACTFF | |
| | EEEKRACKNS YSAWEFVALT KIINEIKSLE KISGEIVPTQ TINEVLNLIL | |
| | DKGSITYKKF RSCINLHESI SFKSLKYDKE NAENAKLIDF RKLVEFKKAL | |
| | GVHSLSRQEL DQISTHITLI KDNVKLKTVL EKYNLSNEQI NNLLEIEFND | |
| | YINLSFKALG MILPLMREGK RYDEACEIAN LKPKTVDEKK DFLPAFCDSI | |
| | FAHELSNPVV NRAISEYRKV LNALLKKYGK VHKIHLELAR DVGLSKKARE | |
| | KIEKEQKENQ AVNAWALKEC ENIGLKASAK NILKLKLWKE QKEICIYSGN | |
| | KISIEHLKDE KALEVDHIYP YSRSFDDSFI NKVLVFTKEN QEKLNKTPFE | |
| | AFGKNIEKWS KIQTLAQNLP YKKKNKILDE NFKDKQQEDF ISRNLNDTRY | |
| | IATLIAKYTK EYLNFLLLSE NENANLKSGE KGSKIHVQTI SGMLTSVLRH | |
| | TWGFDKKDRN NHLHHALDAI IVAYSTNSII KAFSDFRKNQ ELLKARFYAK | |
| | ELTSDNYKHQ VKFFEPFKSF REKILSKIDE IFVSKPPRKR ARRALHKDTF | |
| | HSENKIIDKC SYNSKEGLQI ALSCGRVRKI GTKYVENDTI VRVDIFKKQN | |
| | KFYAIPIYAM DFALGILPNK IVITGKDKNN NPKQWQTIDE SYEFCFSLYK | |
| | NDLILLQKKN MQEPEFAYYN DFSISTSSIC VEKHDNKFEN LTSNQKLLFS | |
| | NAKEGSVKVE SLGIQNLKVF EKYIITPLGD KIKADFQPRE NISLKTSKKY | |
| | GLR | |
| AJN60025.1 | MSDLVLGLDI GIGSVGVGIL NKVTGEIIHK NSRIFPAAQA ENNLVRRTNR | (SEQ |
| GI: 757015981 | QGRRLARRKK HRRVRLNRLF EESGLITDFT KISININPYQ LRVKGLTDEL | ID |
| | SNEELFIALK NMVKHRGISY LDDASDDGNS SVGDYAQIVK ENSKQLETKT | NO: |
| | PGQIQLERYQ TYGQLRGDFT VEKDGKKHRL INVFPTSAYR SEALRILQTQ | 41) |
| | QEFNPQITDE FINRYLEILT GKRKYYHGPG NEKSRTDYGR YRTSGETLDN | |
| | IFGILIGKCT FYPDEFRAAK ASYTAQEFNL LNDLNNLTVP TETKKLSKEQ | |
| | KNQIINYVKN EKAMGPAKLF KYIAKLLSCD VADIKGYRID KSGKAEIHTF | |
| | EAYRKMKTLE TLDIEQMDRE TLDKLAYVLT LNTEREGIQE ALEHEFADGS | |
| | FSQKQVDELV QFRKANSSIF GKGWHNFSVK LMMELIPELY ETSEEQMTIL | |
| | TRLGKQKTTS SSNKTKYIDE KLLTEEIYNP VVAKSVRQAI KIVNAAIKEY | |
| | GDFDNIVIEM ARETNEDDEK KAIQKIQKAN KDEKDAAMLK AANQYNGKAE | |
| | LPHSVFHGHK QLATKIRLWH QQGERCLYTG KTISIHDLIN NSNQFEVDHI | |
| | LPLSITFDDS LANKVLVYAT ANQEKGQRTP YQALDSMDDA WSFRELKAFV | |
| | RESKTLSNKK KEYLLTEEDI SKFDVRKKFI ERNLVDTRYA SRVVLNALQE | |
| | HFRAHKIDTK VSVVRGQFTS QLRRHWGIEK TRDTYHHHAV DALIIAASSQ | |
| | LNLWKKQKNT LVSYSEDQLL DIETGELISD DEYKESVFKA PYQHFVDTLK | |
| | SKEFEDSILF SYQVDSKFNR KISDATIYAT RQAKVGKDKA DETYVLGKIK | |
| | DIYTQDGYDA FMKIYKKDKS KFLMYRHDPQ TFEKVIEPIL ENYPNKQINE | |
| | KGKEVPCNPF LKYKEEHGYI RKYSKKGNGP EIKSLKYYDS KLGNHIDITP | |
| | KDSNNKVVLQ SVSPWRADVY FNKTTGKYEI LGLKYADLQF EKGTGTYKIS | |
| | QEKYNDIKKK EGVDSDSEFK FTLYKNDLLL VKDTETKEQQ LFRFLSRTMP | |
| | KQKHYVELKP YDKQKFEGGE ALIKVLGNVA NSGQCKKGLG KSNISIYKVR | |
| | TDVLGNQHII KNEGDKPKLM | |
| WP_002664048.1 | MKHILGLDLG TNSIGWALIE RNIEEKYGKI IGMGSRIVPM GAELSKFEQG | (SEQ |
| Bergeyella | QAQTKNADRR TNRGARRLNK RYKQRRNKLI YILQKLDMLP SQIKLKEDFS | ID |
| zoohelcum | DPNKIDKITI LPISKKQEQL TAFDLVSLRV KALTEKVGLE DLGKIIYKYN | NO: |
| ATCC 43767 | QLRGYAGGSL EPEKEDIFDE EQSKDKKNKS FIAFSKIVFL GEPQEEIFKN | 42) |
| | KKLNRRAIIV ETEEGNFEGS TFLENIKVGD SLELLINISA SKSGDTITIK | |
| | LPNKTNWRKK MENIENQLKE KSKEMGREFY ISEFLLELLK ENRWAKIRNN | |
| | TILRARYESE FEAIWNEQVK HYPFLENLDK KTLIEIVSFI FPGEKESQKK | |
| | YRELGLEKGL KYIIKNQVVF YQRELKDQSH LISDCRYEPN EKAIAKSHPV | |
| | FQEYKVWEQI NKLIVNTKIE AGTNRKGEKK YKYIDRPIPT ALKEWIFEEL | |
| | QNKKEITFSA IFKKLKAEFD LREGIDFLNG MSPKDKLKGN ETKLQLQKSL | |
| | GELWDVLGLD SINRQIELWN ILYNEKGNEY DLTSDRTSKV LEFINKYGNN | |
| | IVDDNAEETA IRISKIKFAR AYSSLSLKAV ERILPLVRAG KYFNNDFSQQ | |
| | LQSKILKLLN ENVEDPFAKA AQTYLDNNQS VLSEGGVGNS IATILVYDKH | |
| | TAKEYSHDEL YKSYKEINLL KQGDLRNPLV EQIINEALVL IRDIWKNYGI | |
| | KPNEIRVELA RDLKNSAKER ATIHKRNKDN QTINNKIKET LVKNKKELSL | |
| | ANIEKVKLWE AQRHLSPYTG QPIPLSDLFD KEKYDVDHII PISRYFDDSF | |
| | TNKVISEKSV NQEKANRTAM EYFEVGSLKY SIFTKEQFIA HVNEYFSGVK | |
| | RKNLLATSIP EDPVQRQIKD TQYIAIRVKE ELNKIVGNEN VKTTTGSITD | |
| | YLRNHWGLTD KFKLLLKERY EALLESEKFL EAEYDNYKKD FDSRKKEYEE | |
| | KEVLFEEQEL TREEFIKEYK ENYIRYKKNK LIIKGWSKRI DHRHHAIDAL | |
| | IVACTEPAHI KRLNDLNKVL QDWLVEHKSE FMPNFEGSNS ELLEEILSLP | |
| | ENERTEIFTQ IEKFRAIEMP WKGFPEQVEQ KLKEIIISHK PKDKLLLQYN | |
| | KAGDRQIKLR GQLHEGTLYG ISQGKEAYRI PLTKFGGSKF ATEKNIQKIV | |
| | SPFLSGFIAN HLKEYNNKKE EAFSAEGIMD LNNKLAQYRN EKGELKPHTP | |
| | ISTVKIYYKD PSKNKKKKDE EDLSLQKLDR EKAFNEKLYV KTGDNYLFAV | |
| | LEGEIKTKKT SQIKRLYDII SFFDATNFLK EEFRNAPDKK TFDKDLLFRQ | |
| | YFEERNKAKL LFTLKQGDFV YLPNENEEVI LDKESPLYNQ YWGDLKERGK | |
| | NIYVVQKFSK KQIYFIKHTI ADIIKKDVEF GSQNCYETVE GRSIKENCFK | |
| | LEIDRLGNIV KVIKR | |
| CBK78998.1 | MKQEYFLGLD MGTGSLGWAV TDSTYQVMRK HGKALWGTRL FESASTAEER | (SEQ |
| Coprococcus | RMFRTARRRL DRRNWRIQVL QEIFSEEISK VDPGFFLRMK ESKYYPEDKR | ID |
| catus GD/7 | DAEGNCPELP YALFVDDNYT DKNYHKDYPT IYHLRKMLME TTEIPDIRLV | NO: |
| | YLVLHHMMKH RGHFLLSGDI SQIKEFKSTF EQLIQNIQDE ELEWHISLDD | 43) |
| | AAIQFVEHVL KDRNLTRSTK KSRLIKQLNA KSACEKAILN LLSGGTVKLS | |
| | DIFNNKELDE SERPKVSFAD SGYDDYIGIV EAELAEQYYI IASAKAVYDW | |
| | SVLVEILGNS VSISEAKIKV YQKHQADLKT LKKIVRQYMT KEDYKRVFVD | |
| | TEEKLNNYSA YIGMTKKNGK KVDLKSKQCT QADFYDFLKK NVIKVIDHKE | |
| | ITQEIESEIE KENFLPKQVT KDNGVIPYQV HDYELKKILD NLGTRMPFIK | |
| | ENAEKIQQLF EFRIPYYVGP LNRVDDGKDG KFTWSVRKSD ARIYPWNFTE | |
| | VIDVEASAEK FIRRMTNKCT YLVGEDVLPK DSLVYSKFMV LNELNNLRLN | |
| | GEKISVELKQ RIYEELFCKY RKVTRKKLER YLVIEGIAKK GVEITGIDGD | |
| | FKASLTAYHD FKERLTDVQL SQRAKEAIVL NVVLFGDDKK LLKQRLSKMY | |
| | PNLTTGQLKG ICSLSYQGWG RLSKTFLEEI TVPAPGTGEV WNIMTALWQT | |
| | NDNLMQLLSR NYGFTNEVEE FNTLKKETDL SYKTVDELYV SPAVKRQIWQ | |
| | TLKVVKEIQK VMGNAPKRVF VEMAREKQEG KRSDSRKKQL VELYRACKNE | |
| | ERDWITELNA QSDQQLRSDK LFLYYIQKGR CMYSGETIQL DELWDNTKYD | |
| | IDHIYPQSKT MDDSLNNRVL VKKNYNAIKS DTYPLSLDIQ KKMMSFWKML | |
| | QQQGFITKEK YVRLVRSDEL SADELAGFIE RQIVETRQST KAVATILKEA | |
| | LPDTEIVYVK AGNVSNFRQT YELLKVREMN DLHHAKDAYL NIVVGNAYFV | |
| | KFTKNAAWFI RNNPGRSYNL KRMFEFDIER SGEIAWKAGN KGSIVTVKKV | |
| | MQKNNILVTR KAYEVKGGLF DQQIMKKGKG QVPIKGNDER LADIEKYGGY | |
| | NKAAGTYFML VKSLDKKGKE IRTIEFVPLY LKNQIEINHE SAIQYLAQER | |
| | GLNSPEILLS KIKIDTLFKV DGFKMWLSGR TGNQLIFKGA NQLILSHQEA | |
| | AILKGVVKYV NRKNENKDAK LSERDGMTEE KLLQLYDTFL DKLSNTVYSI | |
| | RLSAQIKTLT EKRAKFIGLS NEDQCIVLNE ILHMFQCQSG SANLKLIGGP | |
| | GSAGILVMNN NITACKQISV INQSPTGIYE KEIDLIKL | |
| WP_002235162.1 | MAAFKPNPIN YILGLDIGIA SVGWAMVEID EDENPICLID LGVRVFERAE | (SEQ |
| Neisseria | VPKTGDSLAM ARRLARSVRR LTRRRAHRLL RARRLLKREG VLQAADFDEN | ID |
| meningitidis | GLIKSLPNTP WQLRAAALDR KLTPLEWSAV LLHLIKHRGY LSQRKNEGET | NO: |
| Z2491 | ADKELGALLK GVADNAHALQ TGDFRTPAEL ALNKFEKESG HIRNQRGDYS | 44) |
| | HTFSRKDLQA ELILLFEKQK EFGNPHVSGG LKEGIETLLM TQRPALSGDA | |
| | VQKMLGHCTF EPAEPKAAKN TYTAERFIWL TKLNNLRILE QGSERPLTDT | |
| | ERATLMDEPY RKSKLTYAQA RKLLGLEDTA FFKGLRYGKD NAEASTLMEM | |
| | KAYHAISRAL EKEGLKDKKS PLNLSPELQD EIGTAFSLFK TDEDITGRLK | |
| | DRIQPEILEA LLKHISFDKF VQISLKALRR IVPLMEQGKR YDEACAEIYG | |
| | DHYGKKNTEE KIYLPPIPAD EIRNPVVLRA LSQARKVING VVRRYGSPAR | |
| | IHIETAREVG KSFKDRKEIE KRQEENRKDR EKAAAKFREY FPNFVGEPKS | |
| | KDILKLRLYE QQHGKCLYSG KEINLGRLNE KGYVEIDHAL PFSRTWDDSF | |
| | NNKVLVLGSE NQNKGNQTPY EYFNGKDNSR EWQEFKARVE TSRFPRSKKQ | |
| | RILLQKFDED GFKERNLNDT RYVNRFLCQF VADRMRLTGK GKKRVFASNG | |
| | QITNLLRGFW GLRKVRAEND RHHALDAVVV ACSTVAMQQK ITRFVRYKEM | |
| | NAFDGKTIDK ETGEVLHQKT HFPQPWEFFA QEVMIRVFGK PDGKPEFEEA | |
| | DTPEKLRTLL AEKLSSRPEA VHEYVTPLFV SRAPNRKMSG QGHMETVKSA | |
| | KRLDEGVSVL RVPLTQLKLK DLEKMVNRER EPKLYEALKA RLEAHKDDPA | |
| | KAFAEPFYKY DKAGNRTQQV KAVRVEQVQK TGVWVRNHNG IADNATMVRV | |
| | DVFEKGDKYY LVPIYSWQVA KGILPDRAVV QGKDEEDWQL IDDSFNFKFS | |
| | LHPNDLVEVI TKKARMFGYF ASCHRGTGNI NIRIHDLDHK IGKNGILEGI | |
| | GVKTALSFQK YQIDELGKEI RPCRLKKRPP VR | |
| WP_012414420.1 | MQKNINTKQN HIYIKQAQKI KEKLGDKPYR IGLDLGVGSI GFAIVSMEEN | (SEQ |
| Elusimicrobium | DGNVLLPKEI IMVGSRIFKA SAGAADRKLS RGQRNNHRHT RERMRYLWKV | ID |
| minutum | LAEQKLALPV PADLDRKENS SEGETSAKRF LGDVLQKDIY ELRVKSLDER | NO: |
| Pei191 | LSLQELGYVL YHIAGHRGSS AIRTFENDSE EAQKENTENK KIAGNIKRLM | 45) |
| | AKKNYRTYGE YLYKEFFENK EKHKREKISN AANNHKFSPT RDLVIKEAEA | |
| | ILKKQAGKDG FHKELTEEYI EKLTKAIGYE SEKLIPESGF CPYLKDEKRL | |
| | PASHKLNEER RLWETLNNAR YSDPIVDIVT GEITGYYEKQ FTKEQKQKLF | |
| | DYLLTGSELT PAQTKKLLGL KNTNFEDIIL QGRDKKAQKI KGYKLIKLES | |
| | MPFWARLSEA QQDSFLYDWN SCPDEKLLTE KLSNEYHLTE EEIDNAFNEI | |
| | VLSSSYAPLG KSAMLIILEK IKNDLSYTEA VEEALKEGKL TKEKQAIKDR | |
| | LPYYGAVLQE STQKIIAKGF SPQFKDKGYK TPHTNKYELE YGRIANPVVH | |
| | QTLNELRKLV NEIIDILGKK PCEIGLETAR ELKKSAEDRS KLSREQNDNE | |
| | SNRNRIYEIY IRPQQQVIIT RRENPRNYIL KFELLEEQKS QCPFCGGQIS | |
| | PNDIINNQAD IEHLFPIAES EDNGRNNLVI SHSACNADKA KRSPWAAFAS | |
| | AAKDSKYDYN RILSNVKENI PHKAWRFNQG AFEKFIENKP MAARFKTDNS | |
| | YISKVAHKYL ACLFEKPNII CVKGSLTAQL RMAWGLQGLM IPFAKQLITE | |
| | KESESFNKDV NSNKKIRLDN RHHALDAIVI AYASRGYGNL LNKMAGKDYK | |
| | INYSERNWLS KILLPPNNIV WENIDADLES FESSVKTALK NAFISVKHDH | |
| | SDNGELVKGT MYKIFYSERG YTLTTYKKLS ALKLTDPQKK KTPKDFLETA | |
| | LLKFKGRESE MKNEKIKSAI ENNKRLFDVI QDNLEKAKKL LEEENEKSKA | |
| | EGKKEKNIND ASIYQKAISL SGDKYVQLSK KEPGKFFAIS KPTPTTTGYG | |
| | YDTGDSLCVD LYYDNKGKLC GEIIRKIDAQ QKNPLKYKEQ GFTLFERIYG | |
| | GDILEVDFDI HSDKNSFRNN TGSAPENRVF IKVGTFTEIT NNNIQIWFGN | |
| | IIKSTGGQDD SFTINSMQQY NPRKLILSSC GFIKYRSPIL KNKEG | |
| WP_009105777.1 | MIMKLEKWRL GLDLGTNSIG WSVFSLDKDN SVQDLIDMGV RIFSDGRDPK | (SEQ |
| Treponema sp. | TKEPLAVARR TARSQRKLIY RRKLRRKQVF KFLQEQGLFP KTKEECMTLK | ID |
| JC4 | SLNPYELRIK ALDEKLEPYE LGRALFNLAV RRGFKSNRKD GSREEVSEKK | NO: |
| | SPDEIKTQAD MQTHLEKAIK ENGCRTITEF LYKNQGENGG IRFAPGRMTY | 46) |
| | YPTRKMYEEE FNLIRSKQEK YYPQVDWDDI YKAIFYQRPL KPQQRGYCIY | |
| | ENDKERTFKA MPCSQKLRIL QDIGNLAYYE GGSKKRVELN DNQDKVLYEL | |
| | LNSKDKVTFD QMRKALCLAD SNSFNLEENR DFLIGNPTAV KMRSKNRFGK | |
| | LWDEIPLEEQ DLIIETIITA DEDDAVYEVI KKYDLTQEQR DFIVKNTILQ | |
| | SGTSMLCKEV SEKLVKRLEE IADLKYHEAV ESLGYKFADQ TVEKYDLLPY | |
| | YGKVLPGSTM EIDLSAPETN PEKHYGKISN PTVHVALNQT RVVVNALIKE | |
| | YGKPSQIAIE LSRDLKNNVE KKAEIARKQN QRAKENIAIN DTISALYHTA | |
| | FPGKSFYPNR NDRMKYRLWS ELGLGNKCIY CGKGISGAEL FTKEIEIEHI | |
| | LPFSRTLLDA ESNLTVAHSS CNAFKAERSP FEAFGTNPSG YSWQEIIQRA | |
| | NQLKNTSKKN KFSPNAMDSF EKDSSFIARQ LSDNQYIAKA ALRYLKCLVE | |
| | NPSDVWTTNG SMTKLLRDKW EMDSILCRKF TEKEVALLGL KPEQIGNYKK | |
| | NRFDHRHHAI DAVVIGLTDR SMVQKLATKN SHKGNRIEIP EFPILRSDLI | |
| | EKVKNIVVSF KPDHGAEGKL SKETLLGKIK LHGKETFVCR ENIVSLSEKN | |
| | LDDIVDEKIK SKVKDYVAKH KGQKIEAVLS DFSKENGIKK VRCVNRVQTP | |
| | IEITSGKISR YLSPEDYFAA VIWEIPGEKK TFKAQYIRRN EVEKNSKGLN | |
| | VVKPAVLENG KPHPAAKQVC LLHKDDYLEF SDKGKMYFCR IAGYAATNNK | |
| | LDIRPVYAVS YCADWINSTN ETMLTGYWKP TPTQNWVSVN VLFDKQKARL | |
| | VTVSPIGRVF RK | |
| WP_002460848.1 | MNQKFILGLD IGITSVGYGL IDYETKNIID AGVRLFPEAN VENNEGRRSK | (SEQ |
| Staphylococcus | RGSRRLKRRR IHRLERVKKL LEDYNLLDQS QIPQSTNPYA IRVKGLSEAL | ID |
| lugdunensis | SKDELVIALL HIAKRRGIHK IDVIDSNDDV GNELSTKEQL NKNSKLLKDK | NO: |
| M23590 | FVCQIQLERM NEGQVRGEKN RFKTADIIKE IIQLLNVQKN FHQLDENFIN | 47) |
| | KYIELVEMRR EYFEGPGKGS PYGWEGDPKA WYETLMGHCT YFPDELRSVK | |
| | YAYSADLFNA LNDLNNLVIQ RDGLSKLEYH EKYHIIENVF KQKKKPTLKQ | |
| | IANEINVNPE DIKGYRITKS GKPQFTEFKL YHDLKSVLFD QSILENEDVL | |
| | DQIAEILTIY QDKDSIKSKL TELDILLNEE DKENIAQLTG YTGTHRLSLK | |
| | CIRLVLEEQW YSSRNQMEIF THLNIKPKKI NLTAANKIPK AMIDEFILSP | |
| | VVKRTFGQAI NLINKIIEKY GVPEDIIIEL ARENNSKDKQ KFINEMQKKN | |
| | ENTRKRINEI IGKYGNQNAK RLVEKIRLHD EQEGKCLYSL ESIPLEDLLN | |
| | NPNHYEVDHI IPRSVSFDNS YHNKVLVKQS ENSKKSNLTP YQYFNSGKSK | |
| | LSYNQFKQHI LNLSKSQDRI SKKKKEYLLE ERDINKFEVQ KEFINRNLVD | |
| | TRYATRELTN YLKAYFSANN MNVKVKTING SFTDYLRKVW KFKKERNHGY | |
| | KHHAEDALII ANADFLFKEN KKLKAVNSVL EKPEIESKQL DIQVDSEDNY | |
| | SEMFIIPKQV QDIKDFRNFK YSHRVDKKPN RQLINDTLYS TRKKDNSTYI | |
| | VQTIKDIYAK DNTTLKKQFD KSPEKFLMYQ HDPRTFEKLE VIMKQYANEK | |
| | NPLAKYHEET GEYLTKYSKK NNGPIVKSLK YIGNKLGSHL DVTHQFKSST | |
| | KKLVKLSIKP YRFDVYLTDK GYKFITISYL DVLKKDNYYY IPEQKYDKLK | |
| | LGKAIDKNAK FIASFYKNDL IKLDGEIYKI IGVNSDTRNM IELDLPDIRY | |
| | KEYCELNNIK GEPRIKKTIG KKVNSIEKLT TDVLGNVFTN TQYTKPQLLF | |
| | KRGN | |
| WP_011681470.1 | MTKPYSIGLD IGTNSVGWAV TTDNYKVPSK KMKVLGNTSK KYIKKNLLGV | (SEQ |
| Streptococcus | LLFDSGITAE GRRLKRTARR RYTRRRNRIL YLQEIFSTEM ATLDDAFFQR | ID |
| thermophilus | LDDSFLVPDD KRDSKYPIFG NLVEEKAYHD EFPTIYHLRK YLADSTKKAD | NO: |
| LMD-9 | LRLVYLALAH MIKYRGHFLI EGEFNSKNND IQKNFQDFLD TYNAIFESDL | 48) |
| | SLENSKQLEE IVKDKISKLE KKDRILKLFP GEKNSGIFSE FLKLIVGNQA | |
| | DFRKCFNLDE KASLHFSKES YDEDLETLLG YIGDDYSDVF LKAKKLYDAI | |
| | LLSGFLTVTD NETEAPLSSA MIKRYNEHKE DLALLKEYIR NISLKTYNEV | |
| | FKDDTKNGYA GYIDGKTNQE DFYVYLKKLL AEFEGADYFL EKIDREDFLR | |
| | KQRTFDNGSI PYQIHLQEMR AILDKQAKFY PFLAKNKERI EKILTFRIPY | |
| | YVGPLARGNS DFAWSIRKRN EKITPWNFED VIDKESSAEA FINRMTSFDL | |
| | YLPEEKVLPK HSLLYETFNV YNELTKVRFI AESMRDYQFL DSKQKKDIVR | |
| | LYFKDKRKVT DKDIIEYLHA IYGYDGIELK GIEKQFNSSL STYHDLLNII | |
| | NDKEFLDDSS NEAIIEEIIH TLTIFEDREM IKQRLSKFEN IFDKSVLKKL | |
| | SRRHYTGWGK LSAKLINGIR DEKSGNTILD YLIDDGISNR NFMQLIHDDA | |
| | LSFKKKIQKA QIIGDEDKGN IKEVVKSLPG SPAIKKGILQ SIKIVDELVK | |
| | VMGGRKPESI VVEMARENQY TNQGKSNSQQ RLKRLEKSLK ELGSKILKEN | |
| | IPAKLSKIDN NALQNDRLYL YYLQNGKDMY TGDDLDIDRL SNYDIDHIIP | |
| | QAFLKDNSID NKVLVSSASN RGKSDDVPSL EVVKKRKTFW YQLLKSKLIS | |
| | QRKFDNLTKA ERGGLSPEDK AGFIQRQLVE TRQITKHVAR LLDEKFNNKK | |
| | DENNRAVRTV KIITLKSTLV SQFRKDFELY KVREINDFHH AHDAYLNAVV | |
| | ASALLKKYPK LEPEFVYGDY PKYNSFRERK SATEKVYFYS NIMNIFKKSI | |
| | SLADGRVIER PLIEVNEETG ESVWNKESDL ATVRRVLSYP QVNVVKKVEE | |
| | QNHGLDRGKP KGLFNANLSS KPKPNSNENL VGAKEYLDPK KYGGYAGISN | |
| | SFTVLVKGTI EKGAKKKITN VLEFQGISIL DRINYRKDKL NFLLEKGYKD | |
| | IELIIELPKY SLFELSDGSR RMLASILSTN NKRGEIHKGN QIFLSQKFVK | |
| | LLYHAKRISN TINENHRKYV ENHKKEFEEL FYYILEFNEN YVGAKKNGKL | |
| | LNSAFQSWQN HSIDELCSSF IGPTGSERKG LFELTSRGSA ADFEFLGVKI | |
| | PRYRDYTPSS LLKDATLIHQ SVTGLYETRI DLAKLGEG | |
| WP_009293010.1 | MKRILGLDLG TNSIGWALVN EAENKDERSS IVKLGVRVNP LTVDELTNFE | (SEQ |
| Bacteroides | KGKSITTNAD RTLKRGMRRN LQRYKLRRET LTEVLKEHKL ITEDTILSEN | ID |
| fragilis NCTC | GNRTTFETYR LRAKAVTEEI SLEEFARVLL MINKKRGYKS SRKAKGVEEG | NO: |
| 9343 Cas9 | TLIDGMDIAR ELYNNNLTPG ELCLQLLDAG KKFLPDFYRS DLQNELDRIW | 49) |
| | EKQKEYYPEI LTDVLKEELR GKKRDAVWAI CAKYFVWKEN YTEWNKEKGK | |
| | TEQQEREHKL EGIYSKRKRD EAKRENLQWR VNGLKEKLSL EQLVIVFQEM | |
| | NTQINNSSGY LGAISDRSKE LYFNKQTVGQ YQMEMLDKNP NASLRNMVFY | |
| | RQDYLDEFNM LWEKQAVYHK ELTEELKKEI RDIIIFYQRR LKSQKGLIGF | |
| | CEFESRQIEV DIDGKKKIKT VGNRVISRSS PLFQEFKIWQ ILNNIEVTVV | |
| | GKKRKRRKLK ENYSALFEEL NDAEQLELNG SRRLCQEEKE LLAQELFIRD | |
| | KMTKSEVLKL LFDNPQELDL NFKTIDGNKT GYALFQAYSK MIEMSGHEPV | |
| | DFKKPVEKVV EYIKAVFDLL NWNTDILGFN SNEELDNQPY YKLWHLLYSF | |
| | EGDNTPTGNG RLIQKMTELY GFEKEYATIL ANVSFQDDYG SLSAKAIHKI | |
| | LPHLKEGNRY DVACVYAGYR HSESSLTREE IANKVLKDRL MLLPKNSLHN | |
| | PVVEKILNQM VNVINVIIDI YGKPDEIRVE LARELKKNAK EREELTKSIA | |
| | QTTKAHEEYK TLLQTEFGLT NVSRTDILRY KLYKELESCG YKTLYSNTYI | |
| | SREKLFSKEF DIEHIIPQAR LFDDSFSNKT LEARSVNIEK GNKTAYDFVK | |
| | EKFGESGADN SLEHYLNNIE DLFKSGKISK TKYNKLKMAE QDIPDGFIER | |
| | DLRNTQYIAK KALSMLNEIS HRVVATSGSV TDKLREDWQL IDVMKELNWE | |
| | KYKALGLVEY FEDRDGRQIG RIKDWTKRND HRHHAMDALT VAFTKDVFIQ | |
| | YFNNKNASLD PNANEHAIKN KYFQNGRAIA PMPLREFRAE AKKHLENTLI | |
| | SIKAKNKVIT GNINKTRKKG GVNKNMQQTP RGQLHLETIY GSGKQYLTKE | |
| | EKVNASFDMR KIGTVSKSAY RDALLKRLYE NDNDPKKAFA GKNSLDKQPI | |
| | WLDKEQMRKV PEKVKIVTLE AIYTIRKEIS PDLKVDKVID VGVRKILIDR | |
| | LNEYGNDAKK AFSNLDKNPI WLNKEKGISI KRVTISGISN AQSLHVKKDK | |
| | DGKPILDENG RNIPVDFVNT GNNHHVAVYY RPVIDKRGQL VVDEAGNPKY | |
| | ELEEVVVSFF EAVTRANLGL PIIDKDYKTT EGWQFLFSMK QNEYFVFPNE | |
| | KTGFNPKEID LLDVENYGLI SPNLFRVQKF SLKNYVFRHH LETTIKDTSS | |
| | ILRGITWIDF RSSKGLDTIV KVRVNHIGQI VSVGEY | |
| AOL40912.1 | METQTSNQLI TSHLKDYPKQ DYFVGLDIGT NSVGWAVTNT SYELLKFHSH | (SEQ |
| Veillonella | KMWGSRLFEE GESAVTRRGF RSMRRRLERR KLRLKLLEEL FADAMAQVDS | ID |
| atypica ACS- | TFFIRLHESK YHYEDKTTGH SSKHILFIDE DYTDQDYFTE YPTIYHLRKD | NO: |
| 134-V-Col7a | LMENGTDDIR KLFLAVHHIL KYRGNFLYEG ATFNSNAFTF EDVLKQALVN | 50) |
| | ITFNCFDTNS AISSISNILM ESGKTKSDKA KAIERLVDTY TVFDEVNTPD | |
| | KPQKEQVKED KKTLKAFANL VLGLSANLID LFGSVEDIDD DLKKLQIVGD | |
| | TYDEKRDELA KVWGDEIHII DDCKSVYDAI ILMSIKEPGL TISQSKVKAF | |
| | DKHKEDLVIL KSLLKLDRNV YNEMFKSDKK GLHNYVHYIK QGRTEETSCS | |
| | REDFYKYTKK IVEGLADSKD KEYILNEIEL QTLLPLQRIK DNGVIPYQLH | |
| | LEELKVILDK CGPKFPFLHT VSDGFSVTEK LIKMLEFRIP YYVGPLNTHH | |
| | NIDNGGFSWA VRKQAGRVTP WNFEEKIDRE KSAAAFIKNL TNKCTYLFGE | |
| | DVLPKSSLLY SEFMLLNELN NVRIDGKALA QGVKQHLIDS IFKQDHKKMT | |
| | KNRIELFLKD NNYITKKHKP EITGLDGEIK NDLTSYRDMV RILGNNFDVS | |
| | MAEDIITDIT IFGESKKMLR QTLRNKFGSQ LNDETIKKLS KLRYRDWGRL | |
| | SKKLLKGIDG CDKAGNGAPK TIIELMRNDS YNLMEILGDK FSFMECIEEE | |
| | NAKLAQGQVV NPHDIIDELA LSPAVKRAVW QALRIVDEVA HIKKALPSRI | |
| | FVEVARTNKS EKKKKDSRQK RLSDLYSAIK KDDVLQSGLQ DKEFGALKSG | |
| | LANYDDAALR SKKLYLYYTQ MGRCAYTGNI IDLNQLNTDN YDIDHIYPRS | |
| | LTKDDSFDNL VLCERTANAK KSDIYPIDNR IQTKQKPFWA FLKHQGLISE | |
| | RKYERLTRIA PLTADDLSGF IARQLVETNQ SVKATTTLLR RLYPDIDVVF | |
| | VKAENVSDFR HNNNFIKVRS LNHHHHAKDA YLNIVVGNVY HEKFTRNFRL | |
| | FFKKNGANRT YNLAKMFNYD VICTNAQDGK AWDVKTSMNT VKKMMASNDV | |
| | RVTRRLLEQS GALADATIYK ASVAAKAKDG AYIGMKTKYS VFADVTKYGG | |
| | MTKIKNAYSI IVQYTGKKGE EIKEIVPLPI YLINRNATDI ELIDYVKSVI | |
| | PKAKDISIKY RKLCINQLVK VNGFYYYLGG KTNDKIYIDN AIELVVPHDI | |
| | ATYIKLLDKY DLLRKENKTL KASSITTSIY NINTSTVVSL SNKVGIDVFD | |
| | YFMSKLRTPL YMKMKGNKVD ELSSTGRSKF IKMTLEEQSI YLLEVLNLLT | |
| | NSKTTFDVKP LGITGSRSTI GVKIHNLDEF KIINESITGL YSNEVTIV | |
| WP_013389026.1 | MKYSIGLDIG IASVGWSVIN KDKERIEDMG VRIFQKAENP KDGSSLASSR | (SEQ |
| Ilyobacter | REKRGSRRRN RRKKHRLDRI KNILCESGLV KKNEIEKIYK NAYLKSPWEL | ID |
| polytropus | RAKSLEAKIS NKEIAQILLH IAKRRGFKSF RKTDRNADDT GKLLSGIQEN | NO: |
| DSM 2926 | KKIMEEKGYL TIGDMVAKDP KFNTHVRNKA GSYLFSFSRK LLEDEVRKIQ | 51) |
| | AKQKELGNTH FTDDVLEKYI EVFNSQRNFD EGPSKPSPYY SEIGQIAKMI | |
| | GNCTFESSEK RTAKNTWSGE RFVFLQKLNN FRIVGLSGKR PLTEEERDIV | |
| | EKEVYLKKEV RYEKLRKILY LKEEERFGDL NYSKDEKQDK KTEKTKFISL | |
| | IGNYTIKKLN LSEKLKSEIE EDKSKLDKII EILTFNKSDK TIESNLKKLE | |
| | LSREDIEILL SEEFSGTLNL SLKAIKKILP YLEKGLSYNE ACEKADYDYK | |
| | NNGIKFKRGE LLPVVDKDLI ANPVVLRAIS QTRKVVNAII RKYGTPHTIH | |
| | VEVARDLAKS YDDRQTIIKE NKKRELENEK TKKFISEEFG IKNVKGKLLL | |
| | KYRLYQEQEG RCAYSRKELS LSEVILDESM TDIDHIIPYS RSMDDSYSNK | |
| | VLVLSGENRK KSNLLPKEYF DRQGRDWDTF VLNVKAMKIH PRKKSNLLKE | |
| | KFTREDNKDW KSRALNDTRY ISRFVANYLE NALEYRDDSP KKRVFMIPGQ | |
| | LTAQLRARWR LNKVRENGDL HHALDAAVVA VTDQKAINNI SNISRYKELK | |
| | NCKDVIPSIE YHADEETGEV YFEEVKDTRF PMPWSGFDLE LQKRLESENP | |
| | REEFYNLLSD KRYLGWFNYE EGFIEKLRPV FVSRMPNRGV KGQAHQETIR | |
| | SSKKISNQIA VSKKPLNSIK LKDLEKMQGR DTDRKLYEAL KNRLEEYDDK | |
| | PEKAFAEPFY KPTNSGKRGP LVRGIKVEEK QNVGVYVNGG QASNGSMVRI | |
| | DVFRKNGKFY TVPIYVHQTL LKELPNRAIN GKPYKDWDLI DGSFEFLYSF | |
| | YPNDLIEIEF GKSKSIKNDN KLTKTEIPEV NLSEVLGYYR GMDTSTGAAT | |
| | IDTQDGKIQM RIGIKTVKNI KKYQVDVLGN VYKVKREKRQ TF | |
| WP_005864263.1 | MKKIVGLDLG TNSIGWALIN AYINKEHLYG IEACGSRIIP MDAAILGNFD | (SEQ |
| Parabacteroides | KGNSISQTAD RTSYRGIRRL RERHLLRRER LHRILDLLGF LPKHYSDSLN | ID |
| sp. 20_3 | RYGKFLNDIE CKLPWVKDET GSYKFIFQES FKEMLANFTE HHPILIANNK | NO: |
| | KVPYDWTIYY LRKKALTQKI SKEELAWILL NFNQKRGYYQ LRGEEEETPN | 52) |
| | KLVEYYSLKV EKVEDSGERK GKDTWYNVHL ENGMIYRRTS NIPLDWEGKT | |
| | KEFIVTTDLE ADGSPKKDKE GNIKRSFRAP KDDDWTLIKK KTEADIDKIK | |
| | MTVGAYIYDT LLQKPDQKIR GKLVRTIERK YYKNELYQIL KTQSEFHEEL | |
| | RDKQLYIACL NELYPNNEPR RNSISTRDFC HLFIEDIIFY QRPLKSKKSL | |
| | IDNCPYEENR YIDKESGEIK HASIKCIAKS HPLYQEFRLW QFIVNLRIYR | |
| | KETDVDVTQE LLPTEADYVT LFEWLNEKKE IDQKAFFKYP PFGFKKTTSN | |
| | YRWNYVEDKP YPCNETHAQI IARLGKAHIP KAFLSKEKEE TLWHILYSIE | |
| | DKQEIEKALH SFANKNNLSE EFIEQFKNFP PFKKEYGSYS AKAIKKLLPL | |
| | MRMGKYWSIE NIDNGTRIRI NKIIDGEYDE NIRERVRQKA INLTDITHFR | |
| | ALPLWLACYL VYDRHSEVKD IVKWKTPKDI DLYLKSFKQH SLRNPIVEQV | |
| | ITETLRTVRD IWQQVGHIDE IHIELGREMK NPADKRARMS QQMIKNENTN | |
| | LRIKALLTEF LNPEFGIENV RPYSPSQQDL LRIYEEGVLN SILELPEDIG | |
| | IILGKFNQTD TLKRPTRSEI LRYKLWLEQK YRSPYTGEMI PLSKLFTPAY | |
| | EIEHIIPQSR YFDDSLSNKV ICESEINKLK DRSLGYEFIK NHHGEKVELA | |
| | FDKPVEVLSV EAYEKLVHES YSHNRSKMKK LLMEDIPDQF IERQLNDSRY | |
| | ISKVVKSLLS NIVREENEQE AISKNVIPCT GGITDRLKKD WGINDVWNKI | |
| | VLPRFIRLNE LTESTRFTSI NTNNTMIPSM PLELQKGFNK KRIDHRHHAM | |
| | DAIIIACANR NIVNYLNNVS ASKNTKITRR DLQTLLCHKD KTDNNGNYKW | |
| | VIDKPWETFT QDTLTALQKI TVSFKQNLRV INKTTNHYQH YENGKKIVSN | |
| | QSKGDSWAIR KSMHKETVHG EVNLRMIKTV SFNEALKKPQ AIVEMDLKKK | |
| | ILAMLELGYD TKRIKNYFEE NKDTWQDINP SKIKVYYFTK ETKDRYFAVR | |
| | KPIDTSFDKK KIKESITDTG IQQIMLRHLE TKDNDPTLAF SPDGIDEMNR | |
| | NILILNKGKK HQPIYKVRVY EKAEKFTVGQ KGNKRTKFVE AAKGTNLFFA | |
| | IYETEEIDKD TKKVIRKRSY STIPLNVVIE RQKQGLSSAP EDENGNLPKY | |
| | ILSPNDLVYV PTQEEINKGE VVMPIDRDRI YKMVDSSGIT ANFIPASTAN | |
| | LIFALPKATA EIYCNGENCI QNEYGIGSPQ SKNQKAITGE MVKEICFPIK | |
| | VDRLGNIIQV GSCILTN | |
| GAP01010.1 | MVYDVGLDIG TGSVGWVALD ENGKLARAKG KNLVGVRLFD TAQTAADRRG | (SEQ |
| Fructobacillus | FRTTRRRLSR RKWRLRLLDE LFSAEINEID SSFFQRLKYS YVHPKDEENK | ID |
| fructosus | AHYYGGYLFP TEEETKKFHR SYPTIYHLRQ ELMAQPNKRF DIREIYLAIH | NO: |
| KCTC 3544 | HLVKYRGHFL SSQEKITIGS TYNPEDLANA IEVYADEKGL SWELNNPEQL | 53) |
| | TEIISGEAGY GLNKSMKADE ALKLFEFDNN QDKVAIKTLL AGLTGNQIDF | |
| | AKLFGKDISD KDEAKLWKLK LDDEALEEKS QTILSQLTDE EIELFHAVVQ | |
| | AYDGFVLIGL LNGADSVSAA MVQLYDQHRE DRKLLKSLAQ KAGLKHKRFS | |
| | EIYEQLALAT DEATIKNGIS TARELVEESN LSKEVKEDTL RRLDENEFLP | |
| | KQRTKANSVI PHQLHLAELQ KILQNQGQYY PFLLDTFEKE DGQDNKIEEL | |
| | LRFRIPYYVG PLVTKKDVEH AGGDADNHWV ERNEGFEKSR VTPWNFDKVF | |
| | NRDKAARDFI ERLTGNDTYL IGEKTLPQNS LRYQLFTVLN ELNNVRVNGK | |
| | KFDSKTKADL INDLFKARKT VSLSALKDYL KAQGKGDVTI TGLADESKEN | |
| | SSLSSYNDLK KTFDAEYLEN EDNQETLEKI IEIQTVFEDS KIASRELSKL | |
| | PLDDDQVKKL SQTHYTGWGR LSEKLLDSKI IDERGQKVSI LDKLKSTSQN | |
| | FMSIINNDKY GVQAWITEQN TGSSKLTFDE KVNELTTSPA NKRGIKQSFA | |
| | VLNDIKKAMK EEPRRVYLEF AREDQTSVRS VPRYNQLKEK YQSKSLSEEA | |
| | KVLKKTLDGN KNKMSDDRYF LYFQQQGKDM YTGRPINFER LSQDYDIDHI | |
| | IPQAFTKDDS LDNRVLVSRP ENARKSDSFA YTDEVQKQDG SLWTSLLKSG | |
| | FINRKKYERL TKAGKYLDGQ KTGFIARQLV ETRQIIKNVA SLIEGEYENS | |
| | KAVAIRSEIT ADMRLLVGIK KHREINSFHH AFDALLITAA GQYMQNRYPD | |
| | RDSTNVYNEF DRYTNDYLKN LRQLSSRDEV RRLKSFGFVV GTMRKGNEDW | |
| | SEENTSYLRK VMMFKNILTT KKTEKDRGPL NKETIFSPKS GKKLIPLNSK | |
| | RSDTALYGGY SNVYSAYMTL VRANGKNLLI KIPISIANQI EVGNLKINDY | |
| | IVNNPAIKKF EKILISKLPL GQLVNEDGNL IYLASNEYRH NAKQLWLSTT | |
| | DADKIASISE NSSDEELLEA YDILTSENVK NRFPFFKKDI DKLSQVRDEF | |
| | LDSDKRIAVI QTILRGLQID AAYQAPVKII SKKVSDWHKL QQSGGIKLSD | |
| | NSEMIYQSAT GIFETRVKIS DLL | |
| Bacillus | MNYKMGLDIG IASVGWAVIN LDLKRIEDLG VRIFDKAEHP QNGESLALPR | (SEQ |
| smithii | RIARSARRRL RRRKHRLERI RRLLVSENVL TKEEMNLLFK QKKQIDVWQL | ID |
| WP_003354196.1 | RVDALERKLN NDELARVLLH LAKRRGFKSN RKSERNSKES SEFLKNIEEN | NO: |
| | QSILAQYRSV GEMIVKDSKF AYHKRNKLDS YSNMIARDDL EREIKLIFEK | 54) |
| | QREFNNPVCT ERLEEKYLNI WSSQRPFASK EDIEKKVGFC TFEPKEKRAP | |
| | KATYTFQSFI VWEHINKLRL VSPDETRALT EIERNLLYKQ AFSKNKMTYY | |
| | DIRKLLNLSD DIHFKGLLYD PKSSLKQIEN IRFLELDSYH KIRKCIENVY | |
| | GKDGIRMFNE TDIDTFGYAL TIFKDDEDIV AYLQNEYITK NGKRVSNLAN | |
| | KVYDKSLIDE LLNLSFSKFA HLSMKAIRNI LPYMEQGEIY SKACELAGYN | |
| | FTGPKKKEKA LLLPVIPNIA NPVVMRALTQ SRKVVNAIIK KYGSPVSIHI | |
| | ELARDLSHSF DERKKIQKDQ TENRKKNETA IKQLIEYELT KNPTGLDIVK | |
| | FKLWSEQQGR CMYSLKPIEL ERLLEPGYVE VDHILPYSRS LDDSYANKVL | |
| | VLTKENREKG NHTPVEYLGL GSERWKKFEK FVLANKQFSK KKKQNLLRLR | |
| | YEETEEKEFK ERNLNDTRYI SKFFANFIKE HLKFADGDGG QKVYTINGKI | |
| | TAHLRSRWDF NKNREESDLH HAVDAVIVAC ATQGMIKKIT EFYKAREQNK | |
| | ESAKKKEPIF PQPWPHFADE LKARLSKFPQ ESIEAFALGN YDRKKLESLR | |
| | PVFVSRMPKR SVTGAAHQET LRRCVGIDEQ SGKIQTAVKT KLSDIKLDKD | |
| | GHFPMYQKES DPRTYEAIRQ RLLEHNNDPK KAFQEPLYKP KKNGEPGPVI | |
| | RTVKIIDTKN KVVHLDGSKT VAYNSNIVRT DVFEKDGKYY CVPVYTMDIM | |
| | KGTLPNKAIE ANKPYSEWKE MTEEYTFQFS LFPNDLVRIV LPREKTIKTS | |
| | TNEEIIIKDI FAYYKTIDSA TGGLELISHD RNFSLRGVGS KTLKRFEKYQ | |
| | VDVLGNIHKV KGEKRVGLAA PTNQKKGKTV DSLQSVSD | |
| Mycoplasma | MEKKRKVTLG FDLGIASVGW AIVDSETNQV YKLGSRLFDA PDTNLERRTQ | (SEQ |
| canis PG 14 | RGTRRLLRRR KYRNQKFYNL VKRTEVFGLS SREAIENRFR ELSIKYPNII | ID |
| EIE39736.1 | ELKTKALSQE VCPDEIAWIL HDYLKNRGYF YDEKETKEDF DQQTVESMPS | NO: |
| WP_004794730.1 | YKLNEFYKKY GYFKGALSQP TESEMKDNKD LKEAFFFDFS NKEWLKEINY | 55) |
| | FFNVQKNILS ETFIEEFKKI FSFTRDISKG PGSDNMPSPY GIFGEFGDNG | |
| | QGGRYEHIWD KNIGKCSIFT NEQRAPKYLP SALIFNFLNE LANIRLYSTD | |
| | KKNIQPLWKL SSVDKLNILL NLFNLPISEK KKKLTSTNIN DIVKKESIKS | |
| | IMISVEDIDM IKDEWAGKEP NVYGVGLSGL NIEESAKENK FKFQDLKILN | |
| | VLINLLDNVG IKFEFKDRND IIKNLELLDN LYLFLIYQKE SNNKDSSIDL | |
| | FIAKNESLNI ENLKLKLKEF LLGAGNEFEN HNSKTHSLSK KAIDEILPKL | |
| | LDNNEGWNLE AIKNYDEEIK SQIEDNSSLM AKQDKKYLND NFLKDAILPP | |
| | NVKVTFQQAI LIFNKIIQKF SKDFEIDKVV IELAREMTQD QENDALKGIA | |
| | KAQKSKKSLV EERLEANNID KSVFNDKYEK LIYKIFLWIS QDFKDPYTGA | |
| | QISVNEIVNN KVEIDHIIPY SLCFDDSSAN KVLVHKQSNQ EKSNSLPYEY | |
| | IKQGHSGWNW DEFTKYVKRV FVNNVDSILS KKERLKKSEN LLTASYDGYD | |
| | KLGFLARNLN DTRYATILFR DQLNNYAEHH LIDNKKMFKV IAMNGAVTSF | |
| | IRKNMSYDNK LRLKDRSDFS HHAYDAAIIA LFSNKTKTLY NLIDPSLNGI | |
| | ISKRSEGYWV IEDRYTGEIK ELKKEDWTSI KNNVQARKIA KEIEEYLIDL | |
| | DDEVFFSRKT KRKTNRQLYN ETIYGIATKT DEDGITNYYK KEKFSILDDK | |
| | DIYLRLLRER EKFVINQSNP EVIDQIIEII ESYGKENNIP SRDEAINIKY | |
| | TKNKINYNLY LKQYMRSLTK SLDQFSEEFI NQMIANKTFV LYNPTKNTTR | |
| | KIKFLRLVND VKINDIRKNQ VINKFNGKNN EPKAFYENIN SLGAIVFKNS | |
| | ANNFKTLSIN TQIAIFGDKN WDIEDFKTYN MEKIEKYKEI YGIDKTYNFH | |
| | SFIFPGTILL DKQNKEFYYI SSIQTVRDII EIKFLNKIEF KDENKNQDTS | |
| | KTPKRLMFGI KSIMNNYEQV DISPFGINKK IFE | |
| Odoribacter | METTLGIDLG TNSIGLALVD QEEHQILYSG VRIFPEGINK DTIGLGEKEE | (SEQ |
| laneus YIT | SRNATRRAKR QMRRQYFRKK LRKAKLLELL IAYDMCPLKP EDVRRWKNWD | ID |
| EHP49880.1 | KQQKSTVRQF PDTPAFREWL KQNPYELRKQ AVTEDVTRPE LGRILYQMIQ | NO: |
| | RRGFLSSRKG KEEGKIFTGK DRMVGIDETR KNLQKQTLGA YLYDIAPKNG | 56) |
| | EKYRFRTERV RARYTLRDMY IREFEIIWQR QAGHLGLAHE QATRKKNIFL | |
| | EGSATNVRNS KLITHLQAKY GRGHVLIEDT RITVTFQLPL KEVLGGKIEI | |
| | EEEQLKFKSN ESVLFWQRPL RSQKSLLSKC VFEGRNFYDP VHQKWIIAGP | |
| | TPAPLSHPEF EEFRAYQFIN NIIYGKNEHL TAIQREAVFE LMCTESKDFN | |
| | FEKIPKHLKL FEKFNFDDTT KVPACTTISQ LRKLFPHPVW EEKREEIWHC | |
| | FYFYDDNTLL FEKLQKDYAL QTNDLEKIKK IRLSESYGNV SLKAIRRINP | |
| | YLKKGYAYST AVLLGGIRNS FGKRFEYFKE YEPEIEKAVC RILKEKNAEG | |
| | EVIRKIKDYL VHNRFGFAKN DRAFQKLYHH SQAITTQAQK ERLPETGNLR | |
| | NPIVQQGLNE LRRTVNKLLA TCREKYGPSF KFDHIHVEMG RELRSSKTER | |
| | EKQSRQIREN EKKNEAAKVK LAEYGLKAYR DNIQKYLLYK EIEEKGGTVC | |
| | CPYTGKTLNI SHTLGSDNSV QIEHIIPYSI SLDDSLANKT LCDATFNREK | |
| | GELTPYDFYQ KDPSPEKWGA SSWEEIEDRA FRLLPYAKAQ RFIRRKPQES | |
| | NEFISRQLND TRYISKKAVE YLSAICSDVK AFPGQLTAEL RHLWGLNNIL | |
| | QSAPDITFPL PVSATENHRE YYVITNEQNE VIRLFPKQGE TPRTEKGELL | |
| | LTGEVERKVF RCKGMQEFQT DVSDGKYWRR IKLSSSVTWS PLFAPKPISA | |
| | DGQIVLKGRI EKGVFVCNQL KQKLKTGLPD GSYWISLPVI SQTFKEGESV | |
| | NNSKLTSQQV QLFGRVREGI FRCHNYQCPA SGADGNFWCT LDTDTAQPAF | |
| | TPIKNAPPGV GGGQIILTGD VDDKGIFHAD DDLHYELPAS LPKGKYYGIF | |
| | TVESCDPTLI PIELSAPKTS KGENLIEGNI WVDEHTGEVR FDPKKNREDQ | |
| | RHHAIDAIVI ALSSQSLFQR LSTYNARREN KKRGLDSTEH FPSPWPGFAQ | |
| | DVRQSVVPLL VSYKQNPKTL CKISKTLYKD GKKIHSCGNA VRGQLHKETV | |
| | YGQRTAPGAT EKSYHIRKDI RELKTSKHIG KVVDITIRQM LLKHLQENYH | |
| | IDITQEFNIP SNAFFKEGVY RIFLPNKHGE PVPIKKIRMK EELGNAERLK | |
| | DNINQYVNPR NNHHVMIYQD ADGNLKEEIV SFWSVIERQN QGQPIYQLPR | |
| | EGRNIVSILQ INDTFLIGLK EEEPEVYRND LSTLSKHLYR VQKLSGMYYT | |
| | FRHHLASTLN NEREEFRIQS LEAWKRANPV KVQIDEIGRI TFLNGPLC | |
| Akkermansia | MSRSLTFSFD IGYASIGWAV IASASHDDAD PSVCGCGTVL FPKDDCQAFK | (SEQ |
| muciniphila | RREYRRLRRN IRSRRVRIER IGRLLVQAQI ITPEMKETSG HPAPFYLASE | ID |
| ATCC BAA- | ALKGHRTLAP IELWHVLRWY AHNRGYDNNA SWSNSLSEDG GNGEDTERVK | NO: |
| 835 | HAQDLMDKHG TATMAETICR ELKLEEGKAD APMEVSTPAY KNLNTAFPRL | 57) |
| WP_012421034.1 | IVEKEVRRIL ELSAPLIPGL TAEIIELIAQ HHPLTTEQRG VLLQHGIKLA | |
| | RRYRGSLLFG QLIPRFDNRI ISRCPVTWAQ VYEAELKKGN SEQSARERAE | |
| | KLSKVPTANC PEFYEYRMAR ILCNIRADGE PLSAEIRREL MNQARQEGKL | |
| | TKASLEKAIS SRLGKETETN VSNYFTLHPD SEEALYLNPA VEVLQRSGIG | |
| | QILSPSVYRI AANRLRRGKS VTPNYLLNLL KSRGESGEAL EKKIEKESKK | |
| | KEADYADTPL KPKYATGRAP YARTVLKKVV EEILDGEDPT RPARGEAHPD | |
| | GELKAHDGCL YCLLDTDSSV NQHQKERRLD TMTNNHLVRH RMLILDRLLK | |
| | DLIQDFADGQ KDRISRVCVE VGKELTTFSA MDSKKIQREL TLRQKSHTDA | |
| | VNRLKRKLPG KALSANLIRK CRIAMDMNWT CPFTGATYGD HELENLELEH | |
| | IVPHSFRQSN ALSSLVLTWP_GVNRMKGQRT GYDFVEQEQE NPVPDKPNLH | |
| | ICSLNNYREL VEKLDDKKGH EDDRRRKKKR KALLMVRGLS HKHQSQNHEA | |
| | MKEIGMTEGM MTQSSHLMKL ACKSIKTSLP DAHIDMIPGA VTAEVRKAWD | |
| | VFGVFKELCP EAADPDSGKI LKENLRSLTH LHHALDACVL GLIPYIIPAH | |
| | HNGLLRRVLA MRRIPEKLIP QVRPVANQRH YVINDDGRMM LRDLSASLKE | |
| | NIREQLMEQR VIQHVPADMG GALLKETMQR VLSVDGSGED AMVSLSKKKD | |
| | GKKEKNQVKA SKLVGVFPEG PSKLKALKAA IEIDGNYGVA LDPKPVVIRH | |
| | IKVFKRIMAL KEQNGGKPVR ILKKGMLIHL TSSKDPKHAG VWRIESIQDS | |
| | KGGVKLDLQR AHCAVPKNKT HECNWREVDL ISLLKKYQMK RYPTSYTGTP | |
| | R | |
| Dinoroseobacter | MRLGLDIGTS SIGWWLYETD GAGSDARITG VVDGGVRIFS DGRDPKSGAS | (SEQ |
| shibae DFL | LAVDRRAARA MRRRRDRYLR RRATLMKVLA ETGLMPADPA EAKALEALDP | ID |
| 12 = DSM | FALRAAGLDE PLPLPHLGRA LFHLNQRRGF KSNRKTDRGD NESGKIKDAT | NO: |
| 16493 | ARLDMEMMAN GARTYGEFLH KRRQKATDPR HVPSVRTRLS IANRGGPDGK | 58) |
| WP_012177079.1 | EEAGYDFYPD RRHLEEEFHK LWAAQGAHHP ELTETLRDLL FEKIFFQRPL | |
| | KEPEVGLCLF SGHHGVPPKD PRLPKAHPLT QRRVLYETVN QLRVTADGRE | |
| | ARPLTREERD QVIHALDNKK PTKSLSSMVL KLPALAKVLK LRDGERFTLE | |
| | TGVRDAIACD PLRASPAHPD RFGPRWSILD ADAQWEVISR IRRVQSDAEH | |
| | AALVDWLTEA HGLDRAHAEA TAHAPLPDGY GRLGLTATTR ILYQLTADVV | |
| | TYADAVKACG WHHSDGRTGE CFDRLPYYGE VLERHVIPGS YHPDDDDITR | |
| | FGRITNPTVH IGLNQLRRLV NRIIETHGKP HQIVVELARD LKKSEEQKRA | |
| | DIKRIRDTTE AAKKRSEKLE ELEIEDNGRN RMLLRLWEDL NPDDAMRRFC | |
| | PYTGTRISAA MIFDGSCDVD HILPYSRTLD DSFPNRTLCL REANRQKRNQ | |
| | TPWQAWGDTP HWHAIAANLK NLPENKRWRF APDAMTRFEG ENGFLDRALK | |
| | DTQYLARISR SYLDTLFTKG GHVWVVPGRF TEMLRRHWGL NSLLSDAGRG | |
| | AVKAKNRTDH RHHAIDAAVI AATDPGLLNR ISRAAGQGEA AGQSAELIAR | |
| | DTPPPWEGFR DDLRVRLDRI IVSHRADHGR IDHAARKQGR DSTAGQLHQE | |
| | TAYSIVDDIH VASRTDLLSL KPAQLLDEPG RSGQVRDPQL RKALRVATGG | |
| | KTGKDFENAL RYFASKPGPY QAIRRVRIIK PLQAQARVPV PAQDPIKAYQ | |
| | GGSNHLFEIW RLPDGEIEAQ VITSFEAHTL EGEKRPHPAA KRLLRVHKGD | |
| | MVALERDGRR VVGHVQKMDI ANGLFIVPHN EANADTRNND KSDPFKWIQI | |
| | GARPAIASGI RRVSVDEIGR LRDGGTRPI | |
| Wolinella | MIERILGVDL GISSLGWAIV EYDKDDEAAN RIIDCGVRLF TAAETPKKKE | (SEQ |
| succinogenes | SPNKARREAR GIRRVLNRRR VRMNMIKKLF LRAGLIQDVD LDGEGGMFYS | ID |
| DSM 1740 | KANRADVWEL RHDGLYRLLK GDELARVLIH IAKHRGYKFI GDDEADEESG | NO: |
| WP_011139289.1 | KVKKAGVVLR QNFEAAGCRT VGEWLWRERG ANGKKRNKHG DYEISIHRDL | 59) |
| | LVEEVEAIFV AQQEMRSTIA TDALKAAYRE IAFFVRPMQR IEKMVGHCTY | |
| | FPEERRAPKS APTAEKFIAI SKFFSTVIID NEGWEQKIIE RKTLEELLDF | |
| | AVSREKVEFR HLRKFLDLSD NEIFKGLHYK GKPKTAKKRE ATLFDPNEPT | |
| | ELEFDKVEAE KKAWISLRGA AKLREALGNE FYGRFVALGK HADEATKILT | |
| | YYKDEGQKRR ELTKLPLEAE MVERLVKIGF SDFLKLSLKA IRDILPAMES | |
| | GARYDEAVLM LGVPHKEKSA ILPPLNKTDI DILNPTVIRA FAQFRKVANA | |
| | LVRKYGAFDR VHFELAREIN TKGEIEDIKE SQRKNEKERK EAADWIAETS | |
| | FQVPLTRKNI LKKRLYIQQD GRCAYTGDVI ELERLFDEGY CEIDHILPRS | |
| | RSADDSFANK VLCLARANQQ KTDRTPYEWF GHDAARWNAF ETRTSAPSNR | |
| | VRTGKGKIDR LLKKNFDENS EMAFKDRNLN DTRYMARAIK TYCEQYWVFK | |
| | NSHTKAPVQV RSGKLTSVLR YQWGLESKDR ESHTHHAVDA IIIAFSTQGM | |
| | VQKLSEYYRF KETHREKERP KLAVPLANFR DAVEEATRIE NTETVKEGVE | |
| | VKRLLISRPP RARVTGQAHE QTAKPYPRIK QVKNKKKWRL APIDEEKFES | |
| | FKADRVASAN QKNFYETSTI PRVDVYHKKG KFHLVPIYLH EMVLNELPNL | |
| | SLGTNPEAMD ENFFKFSIFK DDLISIQTQG TPKKPAKIIM GYFKNMHGAN | |
| | MVLSSINNSP CEGFTCTPVS MDKKHKDKCK LCPEENRIAG RCLQGFLDYW | |
| | SQEGLRPPRK EFECDQGVKF ALDVKKYQID PLGYYYEVKQ EKRLGTIPQM | |
| | RSAKKLVKK | |
| Parasutterella | MGKTHIIGVG LDLGGTYTGT FITSHPSDEA EHRDHSSAFT VVNSEKLSFS | (SEQ |
| excrementiho | SKSRTAVRHR VRSYKGFDLR RRLLLLVAEY QLLQKKQTLA PEERENLRIA | ID |
| minis YIT | LSGYLKRRGY ARTEAETDTS VLESLDPSVF SSAPSFTNFF NDSEPLNIQW | NO: |
| 11859 | EAIANSPETT KALNKELSGQ KEADFKKYIK TSFPEYSAKE ILANYVEGRR | 60) |
| WP_008864843.1 | AILDASKYIA NLQSLGHKHR SKYLSDILQD MKRDSRITRL SEAFGSTDNL | |
| | WRIIGNISNL QERAVRWYFN DAKFEQGQEQ LDAVKLKNVL VRALKYLRSD | |
| | DKEWSASQKQ IIQSLEQSGD VLDVLAGLDP DRTIPPYEDQ NNRRPPEDQT | |
| | LYLNPKALSS EYGEKWKSWA NKFAGAYPLL TEDLTEILKN TDRKSRIKIR | |
| | SDVLPDSDYR LAYILQRAFD RSIALDECSI RRTAEDFENG VVIKNEKLED | |
| | VLSGHQLEEF LEFANRYYQE TAKAKNGLWF PENALLERAD LHPPMKNKIL | |
| | NVIVGQALGV SPAEGTDFIE EIWNSKVKGR STVRSICNAI ENERKTYGPY | |
| | FSEDYKFVKT ALKEGKTEKE LSKKFAAVIK VLKMVSEVVP FIGKELRLSD | |
| | EAQSKFDNLY SLAQLYNLIE TERNGFSKVS LAAHLENAWR MTMTDGSAQC | |
| | CRLPADCVRP FDGFIRKAID RNSWEVAKRI AEEVKKSVDF TNGTVKIPVA | |
| | IEANSFNFTA SLTDLKYIQL KEQKLKKKLE DIQRNEENQE KRWLSKEERI | |
| | RADSHGICAY TGRPLDDVGE IDHIIPRSLT LKKSESIYNS EVNLIFVSAQ | |
| | GNQEKKNNIY LLSNLAKNYL AAVFGTSDLS QITNEIESTV LQLKAAGRLG | |
| | YFDLLSEKER ACARHALFLN SDSEARRAVI DVLGSRRKAS VNGTQAWFVR | |
| | SIFSKVRQAL AAWTQETGNE LIFDAISVPA ADSSEMRKRF AEYRPEFRKP | |
| | KVQPVASHSI DAMCIYLAAC SDPFKTKRMG SQLAIYEPIN FDNLFTGSCQ | |
| | VIQNTPRNFS DKTNIANSPI FKETIYAERF LDIIVSRGEI FIGYPSNMPF | |
| | EEKPNRISIG GKDPFSILSV LGAYLDKAPS SEKEKLTIYR VVKNKAFELF | |
| | SKVAGSKFTA EEDKAAKILE ALHFVTVKQD VAATVSDLIK SKKELSKDSI | |
| | ENLAKQKGCL KKVEYSSKEF KFKGSLIIPA AVEWGKVLWN VFKENTAEEL | |
| | KDENALRKAL EAAWPSSFGT RNLHSKAKRV FSLPVVATQS GAVRIRRKTA | |
| | FGDFVYQSQD TNNLYSSFPV KNGKLDWSSP IIHPALQNRN LTAYGYRFVD | |
| | HDRSISMSEF REVYNKDDLM RIELAQGTSS RRYLRVEMPG EKFLAWFGEN | |
| | SISLGSSFKF SVSEVFDNKI YTENAEFTKF LPKPREDNKH NGTIFFELVG | |
| | PRVIFNYIVG GAASSLKEIF SEAGKERS | |
| Streptococcus | MTKFNKNYSI GLDIGVSSVG YAVVTEDYRV PAFKFKVLGN TEKEKIKKNL | (SEQ |
| sanguinis | IGSTTFVSAQ PAKGTRVFRV NRRRIDRRNH RITYLRDIFQ KEIEKVDKNF | ID |
| SK49 | YRRLDESFRV LGDKSEDLQI KQPFFGDKEL ETAYHKKYPT IYHLRKHLAD | NO: |
| WP_002933589.1 | ADKNSPVADI REVYMAISHI LKYRGHFLTL DKINPNNINM QNSWIDFIES | 61) |
| | CQEVFDLEIS DESKNIADIF KSSENRQEKV KKILPYFQQE LLKKDKSIFK | |
| | QLLQLLFGLK TKFKDCFELE EEPDLNFSKE NYDENLENFL GSLEEDFSDV | |
| | FAKLKVLRDT ILLSGMLTYT GATHARFSAT MVERYEEHRK DLQRFKFFIK | |
| | QNLSEQDYLD IFGRKTQNGF DVDKETKGYV GYITNKMVLT NPQKQKTIQQ | |
| | NFYDYISGKI TGIEGAEYFL NKISDGTFLR KLRTSDNGAI PNQIHAYELE | |
| | KIIERQGKDY PFLLENKDKL LSILTFKIPY YVGPLAKGSN SRFAWIKRAT | |
| | SSDILDDNDE DTRNGKIRPW NYQKLINMDE TRDAFITNLI GNDIILLNEK | |
| | VLPKRSLIYE EVMLQNELTR VKYKDKYGKA HFFDSELRQN IINGLFKNNS | |
| | KRVNAKSLIK YLSDNHKDLN AIEIVSGVEK GKSFNSTLKT YNDLKTIFSE | |
| | ELLDSEIYQK ELEEIIKVIT VFDDKKSIKN YLTKFFGHLE ILDEEKINQL | |
| | SKLRYSGWGR YSAKLLLDIR DEDTGFNLLQ FLRNDEENRN LTKLISDNTL | |
| | SFEPKIKDIQ SKSTIEDDIF DEIKKLAGSP AIKRGILNSI KIVDELVQII | |
| | GYPPHNIVIE MARENMTTEE GQKKAKTRKT KLESALKNIE NSLLENGKVP | |
| | HSDEQLQSEK LYLYYLQNGK DMYTLDKTGS PAPLYLDQLD QYEVDHIIPY | |
| | SFLPIDSIDN KVLTHRENNQ QKLNNIPDKE TVANMKPFWE KLYNAKLISQ | |
| | TKYQRLTTSE RTPDGVLTES MKAGFIERQL VETRQIIKHV ARILDNRFSD | |
| | TKIITLKSQL ITNFRNTFHI AKIRELNDYH HAHDAYLAVV VGQTLLKVYP | |
| | KLAPELIYGH HAHFNRHEEN KATLRKHLYS NIMRFFNNPD SKVSKDIWDC | |
| | NRDLPIIKDV IYNSQINFVK RTMIKKGAFY NQNPVGKFNK QLAANNRYPL | |
| | KTKALCLDTS IYGGYGPMNS ALSIIIIAER FNEKKGKIET VKEFHDIFII | |
| | DYEKFNNNPF QFLNDTSENG FLKKNNINRV LGFYRIPKYS LMQKIDGTRM | |
| | LFESKSNLHK ATQFKLTKTQ NELFFHMKRL LTKSNLMDLK SKSAIKESQN | |
| | FILKHKEEFD NISNQLSAFS QKMLGNTTSL KNLIKGYNER KIKEIDIRDE | |
| | TIKYFYDNFI KMFSFVKSGA PKDINDFFDN KCTVARMRPK PDKKLLNATL | |
| | IHQSITGLYE TRIDLSKLGE D | |
| Actinomyces | MLHCIAVIRV PPSEEPGFFE THADSCALCH HGCMTYAAND KAIRYRVGID | (SEQ |
| sp. oral taxon | VGLRSIGFCA VEVDDEDHPI RILNSVVHVH DAGTGGPGET ESLRKRSGVA | ID |
| 180 str. F0310 | ARARRRGRAE KQRLKKLDVL LEELGWGVSS NELLDSHAPW HIRKRLVSEY | NO: |
| AOL41039.1 | IEDETERRQC LSVAMAHIAR HRGWRNSFSK VDTLLLEQAP SDRMQGLKER | 62) |
| | VEDRTGLQFS EEVTQGELVA TLLEHDGDVT IRGFVRKGGK ATKVHGVLEG | |
| | KYMQSDLVAE LRQICRTQRV SETTFEKLVL SIFHSKEPAP SAARQRERVG | |
| | LDELQLALDP AAKQPRAERA HPAFQKFKVV ATLANMRIRE QSAGERSLTS | |
| | EELNRVARYL LNHTESESPT WDDVARKLEV PRHRLRGSSR ASLETGGGLT | |
| | YPPVDDTTVR VMSAEVDWLA DWWDCANDES RGHMIDAISN GCGSEPDDVE | |
| | DEEVNELISS ATAEDMLKLE LLAKKLPSGR VAYSLKTLRE VTAAILETGD | |
| | DLSQAITRLY GVDPGWVPTP APIEAPVGNP SVDRVLKQVA RWLKFASKRW | |
| | GVPQTVNIEH TREGLKSASL LEEERERWER FEARREIRQK EMYKRLGISG | |
| | PFRRSDQVRY EILDLQDCAC LYCGNEINFQ TFEVDHIIPR VDASSDSRRT | |
| | NLAAVCHSCN SAKGGLAFGQ WVKRGDCPSG VSLENAIKRV RSWSKDRLGL | |
| | TEKAMGKRKS EVISRLKTEM PYEEFDGRSM ESVAWMAIEL KKRIEGYFNS | |
| | DRPEGCAAVQ VNAYSGRLTA CARRAAHVDK RVRLIRLKGD DGHHKNRFDR | |
| | RNHAMDALVI ALMTPAIART IAVREDRREA QQLTRAFESW KNFLGSEERM | |
| | QDRWESWIGD VEYACDRLNE LIDADKIPVT ENLRLRNSGK LHADQPESLK | |
| | KARRGSKRPR PQRYVLGDAL PADVINRVTD PGLWTALVRA PGFDSQLGLP | |
| | ADLNRGLKLR GKRISADFPI DYFPTDSPAL AVQGGYVGLE FHHARLYRII | |
| | GPKEKVKYAL LRVCAIDLCG IDCDDLFEVE LKPSSISMRT ADAKLKEAMG | |
| | NGSAKQIGWL VLGDEIQIDP TKFPKQSIGK FLKECGPVSS WRVSALDTPS | |
| | KITLKPRLLS NEPLLKTSRV GGHESDLVVA ECVEKIMKKT GWVVEINALC | |
| | QSGLIRVIRR NALGEVRTSP KSGLPISLNL R | |
| Rhodovulum | MGIRFAFDLG TNSIGWAVWR TGPGVFGEDT AASLDGSGVL IFKDGRNPKD | (SEQ |
| sp. PH10 | GQSLATMRRV PRQSRKRRDR FVLRRRDLLA ALRKAGLFPV DVEEGRRLAA | ID |
| WP_008386983.1 | TDPYHLRAKA LDESLTPHEM GRVIFHLNQR RGFRSNRKAD RQDREKGKIA | NO: |
| | EGSKRLAETL AATNCRTLGE FLWSRHRGTP RTRSPTRIRM EGEGAKALYA | 63) |
| | FYPTREMVRA EFERLWTAQS RFAPDLLTPE RHEEIAGILF RQRDLAPPKI | |
| | GCCTFEPSER RLPRALPSVE ARGIYERLAH LRITTGPVSD RGLTRPERDV | |
| | LASALLAGKS LTFKAVRKTL KILPHALVNF EEAGEKGLDG ALTAKLLSKP | |
| | DHYGAAWHGL SFAEKDTFVG KLLDEADEER LIRRLVTENR LSEDAARRCA | |
| | SIPLADGYGR LGRTANTEIL AALVEETDET GTVVTYAEAV RRAGERTGRN | |
| | WHHSDERDGV ILDRLPYYGE ILQRHVVPGS GEPEEKNEAA RWGRLANPTV | |
| | HIGLNQLRKV VNRLIAAHGR PDQIVVELAR ELKLNREQKE RLDRENRKNR | |
| | EENERRTAIL AEHGQRDTAE NKIRLRLFEE QARANAGIAL CPYTGRAIGI | |
| | AELFTSEVEI DHILPVSLTL DDSLANRVLC RREANREKRR QTPFQAFGAT | |
| | PAWNDIVARA AKLPPNKRWR FDPAALERFE REGGELGRQL NETKYLSRLA | |
| | KIYLGKICDP DRVYVTPGTL TGLLRARWGL NSILSDSNFK NRSDHRHHAV | |
| | DAVVIGVLTR GMIQRIAHDA ARAEDQDLDR VFRDVPVPFE DFRDHVRERV | |
| | STITVAVKPE HGKGGALHED TSYGLVPDTD PNAALGNLVV RKPIRSLTAG | |
| | EVDRVRDRAL RARLGALAAP FRDESGRVRD AKGLAQALEA FGAENGIRRV | |
| | RILKPDASVV TIADRRTGVP YRAVAPGENH HVDIVQMRDG SWRGFAASVF | |
| | EVNRPGWRPE WEVKKLGGKL VMRLHKGDMV ELSDKDGQRR VKVVQQIEIS | |
| | ANRVRLSPHN DGGKLQDRHA DADDPFRWDL ATIPLLKDRG CVAVRVDPIG | |
| | VVTLRRSNV | |
| Bifidobacterium | MSRKNYVDDY AISLDIGNAS VGWSAFTPNY RLVRAKGHEL IGVRLFDPAD | (SEQ |
| bifidum S17 | TAESRRMART TRRRYSRRRW RLRLLDALFD QALSEIDPSF LARRKYSWVH | ID |
| WP_013362995.1 | PDDENNADCW YGSVLFDSNE QDKRFYEKYP TIYHLRKALM EDDSQHDIRE | NO: |
| | IYLAIHHMVK YRGNFLVEGT LESSNAFKED ELLKLLGRIT RYEMSEGEQN | 64) |
| | SDIEQDDENK LVAPANGQLA DALCATRGSR SMRVDNALEA LSAVNDLSRE | |
| | QRAIVKAIFA GLEGNKLDLA KIFVSKEFSS ENKKILGIYF NKSDYEEKCV | |
| | QIVDSGLLDD EEREFLDRMQ GQYNAIALKQ LLGRSTSVSD SKCASYDAHR | |
| | ANWNLIKLQL RTKENEKDIN ENYGILVGWK IDSGQRKSVR GESAYENMRK | |
| | KANVFFKKMI ETSDLSETDK NRLIHDIEED KLFPIQRDSD NGVIPHQLHQ | |
| | NELKQIIKKQ GKYYPFLLDA FEKDGKQINK IEGLLTFRVP YFVGPLVVPE | |
| | DLQKSDNSEN HWMVRKKKGE ITPWNFDEMV DKDASGRKFI ERLVGTDSYL | |
| | LGEPTLPKNS LLYQEYEVLN ELNNVRLSVR TGNHWNDKRR MRLGREEKTL | |
| | LCQRLFMKGQ TVTKRTAENL LRKEYGRTYE LSGLSDESKF TSSLSTYGKM | |
| | CRIFGEKYVN EHRDLMEKIV ELQTVFEDKE TLLHQLRQLE GISEADCALL | |
| | VNTHYTGWGR LSRKLLTTKA GECKISDDFA PRKHSIIEIM RAEDRNLMEI | |
| | ITDKQLGFSD WIEQENLGAE NGSSLMEVVD DLRVSPKVKR GIIQSIRLID | |
| | DISKAVGKRP SRIFLELADD IQPSGRTISR KSRLQDLYRN ANLGKEFKGI | |
| | ADELNACSDK DLQDDRLFLY YTQLGKDMYT GEELDLDRLS SAYDIDHIIP | |
| | QAVTQNDSID NRVLVARAEN ARKTDSFTYM PQIADRMRNF WQILLDNGLI | |
| | SRVKFERLTR QNEFSEREKE RFVQRSLVET RQIMKNVATL MRQRYGNSAA | |
| | VIGLNAELTK EMHRYLGFSH KNRDINDYHH AQDALCVGIA GQFAANRGFF | |
| | ADGEVSDGAQ NSYNQYLRDY LRGYREKLSA EDRKQGRAFG FIVGSMRSQD | |
| | EQKRVNPRTG EVVWSEEDKD YLRKVMNYRK MLVTQKVGDD FGALYDETRY | |
| | AATDPKGIKG IPFDGAKQDT SLYGGFSSAK PAYAVLIESK GKTRLVNVTM | |
| | QEYSLLGDRP SDDELRKVLA KKKSEYAKAN ILLRHVPKMQ LIRYGGGLMV | |
| | IKSAGELNNA QQLWLPYEEY CYFDDLSQGK GSLEKDDLKK LLDSILGSVQ | |
| | CLYPWHRFTE EELADLHVAF DKLPEDEKKN VITGIVSALH ADAKTANLSI | |
| | VGMTGSWRRM NNKSGYTFSD EDEFIFQSPS GLFEKRVTVG ELKRKAKKEV | |
| | NSKYRTNEKR LPTLSGASQP | |
| Barnesiella | MKNILGLDLG LSSIGWSVIR ENSEEQELVA MGSRVVSLTA AELSSFTQGN | (SEQ |
| intestinihominis | GVSINSQRTQ KRTQRKGYDR YQLRRTLLRN KLDTLGMLPD DSLSYLPKLQ | ID |
| YIT 11860 | LWGLRAKAVT QRIELNELGR VLLHLNQKRG YKSIKSDFSG DKKITDYVKT | NO: |
| WP_008863245.1 | VKTRYDELKE MRLTIGELFF RRLTENAFFR CKEQVYPRQA YVEEFDCIMN | 65) |
| | CQRKFYPDIL TDETIRCIRD EIIYYQRPLK SCKYLVSRCE FEKRFYLNAA | |
| | GKKTEAGPKV SPRTSPLFQV CRLWESINNI VVKDRRNEIV FISAEQRAAL | |
| | FDFLNTHEKL KGSDLLKLLG LSKTYGYRLG EQFKTGIQGN KTRVEIERAL | |
| | GNYPDKKRLL QFNLQEESSS MVNTETGEII PMISLSFEQE PLYRLWHVLY | |
| | SIDDREQLQS VLRQKFGIDD DEVLERLSAI DLVKAGFGNK SSKAIRRILP | |
| | FLQLGMNYAE ACEAAGYNHS NNYTKAENEA RALLDRLPAI KKNELRQPVV | |
| | EKILNQMVNV VNALMEKYGR FDEIRVELAR ELKQSKEERS NTYKSINKNQ | |
| | RENEQIAKRI VEYGVPTRSR IQKYKMWEES KHCCIYCGQP VDVGDFLRGF | |
| | DVEVEHIIPK SLYFDDSFAN KVCSCRSCNK EKNNRTAYDY MKSKGEKALS | |
| | DYVERVNTMY TNNQISKTKW QNLLTPVDKI SIDFIDRQLR ESQYIARKAK | |
| | EILTSICYNV TATSGSVTSF LRHVWGWDTV LHDLNFDRYK KVGLTEVIEV | |
| | NHRGSVIRRE QIKDWSKRFD HRHHAIDALT IACTKQAYIQ RLNNLRAEEG | |
| | PDFNKMSLER YIQSQPHFSV AQVREAVDRI LVSFRAGKRA VTPGKRYIRK | |
| | NRKRISVQSV LIPRGALSEE SVYGVIHVWE KDEQGHVIQK QRAVMKYPIT | |
| | SINREMLDKE KVVDKRIHRI LSGRLAQYND NPKEAFAKPV YIDKECRIPI | |
| | RTVRCFAKPA INTLVPLKKD DKGNPVAWVN PGNNHHVAIY RDEDGKYKER | |
| | TVTFWEAVDR CRVGIPAIVT QPDTIWDNIL QRNDISENVL ESLPDVKWQF | |
| | VLSLQQNEMF ILGMNEEDYR YAMDQQDYAL LNKYLYRVQK LSKSDYSFRY | |
| | HTETSVEDKY DGKPNLKLSM QMGKLKRVSI KSLLGLNPHK VHISVLGEIK | |
| | EIS | |
| Aminomonas | MIGEHVRGGC LFDDHWTPNW GAFRLPNTVR TFTKAENPKD GSSLAEPRRQ | (SEQ |
| paucivorans | ARGLRRRLRR KTQRLEDLRR LLAKEGVLSL SDLETLFRET PAKDPYQLRA | ID |
| DSM 12260 | EGLDRPLSFP EWVRVLYHIT KHRGFQSNRR NPVEDGQERS RQEEEGKLLS | NO: |
| WP_006299850.1 | GVGENERLLR EGGYRTAGEM LARDPKFQDH RRNRAGDYSH TLSRSLLLEE | 66) |
| | ARRLFQSQRT LGNPHASSNL EEAFLHLVAF QNPFASGEDI RNKAGHCSLE | |
| | PDQIRAPRRS ASAETFMLLQ KTGNLRLIHR RTGEERPLTD KEREQIHLLA | |
| | WKQEKVTHKT LRRHLEIPEE WLFTGLPYHR SGDKAEEKLF VHLAGIHEIR | |
| | KALDKGPDPA VWDTLRSRRD LLDSIADTLT FYKNEDEILP RLESLGLSPE | |
| | NARALAPLSF SGTAHLSLSA LGKLLPHLEE GKSYTQARAD AGYAAPPPDR | |
| | HPKLPPLEEA DWRNPVVFRA LTQTRKVVNA LVRRYGPPWC IHLETARELS | |
| | QPAKVRRRIE TEQQANEKKK QQAEREFLDI VGTAPGPGDL LKMRLWREQG | |
| | GFCPYCEEYL NPTRLAEPGY AEMDHILPYS RSLDNGWHNR VLVHGKDNRD | |
| | KGNRTPFEAF GGDTARWDRL VAWVQASHLS APKKRNLLRE DFGEEAEREL | |
| | KDRNLTDTRF ITKTAATLLR DRLTFHPEAP KDPVMTLNGR LTAFLRKQWG | |
| | LHKNRKNGDL HHALDAAVLA VASRSFVYRL SSHNAAWGEL PRGREAENGF | |
| | SLPYPAFRSE VLARLCPTRE EILLRLDQGG VGYDEAFRNG LRPVFVSRAP | |
| | SRRLRGKAHM ETLRSPKWKD HPEGPRTASR IPLKDLNLEK LERMVGKDRD | |
| | RKLYEALRER LAAFGGNGKK AFVAPFRKPC RSGEGPLVRS LRIFDSGYSG | |
| | VELRDGGEVY AVADHESMVR VDVYAKKNRF YLVPVYVADV ARGIVKNRAI | |
| | VAHKSEEEWD LVDGSFDFRF SLFPGDLVEI EKKDGAYLGY YKSCHRGDGR | |
| | LLLDRHDRMP RESDCGTFYV STRKDVLSMS KYQVDPLGEI RLVGSEKPPF | |
| | VL | |
| Ralstonia | MAEKQHRWGL DIGTNSIGWA VIALIEGRPA GLVATGSRIF SDGRNPKDGS | (SEQ |
| syzygii R24 | SLAVERRGPR QMRRRRDRYL RRRDRFMQAL INVGLMPGDA AARKALVTEN | ID |
| CCA84553.1 | PYVLRQRGLD QALTLPEFGR ALFHLNQRRG FQSNRKTDRA TAKESGKVKN | NO: |
| | AIAAFRAGMG NARTVGEALA RRLEDGRPVR ARMVGQGKDE HYELYIAREW | 67) |
| | IAQEFDALWA SQQRFHAEVL ADAARDRLRA ILLFQRKLLP VPVGKCFLEP | |
| | NQPRVAAALP SAQRFRLMQE LNHLRVMTLA DKRERPLSFQ ERNDLLAQLV | |
| | ARPKCGFDML RKIVFGANKE AYRFTIESER RKELKGCDTA AKLAKVNALG | |
| | TRWQALSLDE QDRLVCLLLD GENDAVLADA LREHYGLTDA QIDTLLGLSF | |
| | EDGHMRLGRS ALLRVLDALE SGRDEQGLPL SYDKAVVAAG YPAHTADLEN | |
| | GERDALPYYG ELLWRYTQDA PTAKNDAERK FGKIANPTVH IGLNQLRKLV | |
| | NALIQRYGKP AQIVVELARN LKAGLEEKER IKKQQTANLE RNERIRQKLQ | |
| | DAGVPDNREN RLRMRLFEEL GQGNGLGTPC IYSGRQISLQ RLFSNDVQVD | |
| | HILPFSKTLD DSFANKVLAQ HDANRYKGNR GPFEAFGANR DGYAWDDIRA | |
| | RAAVLPRNKR NRFAETAMQD WLHNETDFLA RQLTDTAYLS RVARQYLTAI | |
| | CSKDDVYVSP GRLTAMLRAK WGLNRVLDGV MEEQGRPAVK NRDDHRHHAI | |
| | DAVVIGATDR AMLQQVATLA ARAREQDAER LIGDMPTPWP_NFLEDVRAAV | |
| | ARCVVSHKPD HGPEGGLHND TAYGIVAGPF EDGRYRVRHR VSLFDLKPGD | |
| | LSNVRCDAPL QAELEPIFEQ DDARAREVAL TALAERYRQR KVWLEELMSV | |
| | LPIRPRGEDG KTLPDSAPYK AYKGDSNYCY ELFINERGRW DGELISTFRA | |
| | NQAAYRRFRN DPARFRRYTA GGRPLLMRLC INDYIAVGTA AERTIFRVVK | |
| | MSENKITLAE HFEGGTLKQR DADKDDPFKY LTKSPGALRD LGARRIFVDL | |
| | IGRVLDPGIK GD | |
| Catenibacterium | IVDYCIGLDL GTGSVGWAVV DMNHRLMKRN GKHLWGSRLF SNAETAANRR | (SEQ |
| mitsuokai | ASRSIRRRYN KRRERIRLLR AILQDMVLEK DPTFFIRLEH TSFLDEEDKA | ID |
| DSM 15897 | KYLGTDYKDN YNLFIDEDFN DYTYYHKYPT IYHLRKALCE STEKADPRLI | NO: |
| WP_006506696.1 | YLALHHIVKY RGNFLYEGQK FNMDASNIED KLSDIFTQFT SFNNIPYEDD | 68) |
| | EKKNLEILEI LKKPLSKKAK VDEVMTLIAP EKDYKSAFKE LVTGIAGNKM | |
| | NVTKMILCEP IKQGDSEIKL KFSDSNYDDQ FSEVEKDLGE YVEFVDALHN | |
| | VYSWVELQTI MGATHTDNAS ISEAMVSRYN KHHDDLKLLK DCIKNNVPNK | |
| | YFDMFRNDSE KSKGYYNYIN RPSKAPVDEF YKYVKKCIEK VDTPEAKQIL | |
| | NDIELENFLL KQNSRINGSV PYQMQLDEMI KIIDNQAEYY PILKEKREQL | |
| | LSILTFRIPY YFGPLNETSE HAWIKRLEGK ENQRILPWNY QDIVDVDATA | |
| | EGFIKRMRSY CTYFPDEEVL PKNSLIVSKY EVYNELNKIR VDDKLLEVDV | |
| | KNDIYNELFM KNKTVTEKKL KNWLVNNQCC SKDAEIKGFQ KENQFSTSLT | |
| | PWIDFTNIFG KIDQSNFDLI ENIIYDLTVF EDKKIMKRRL KKKYALPDDK | |
| | VKQILKLKYK DWSRLSKKLL DGIVADNRFG SSVTVLDVLE MSRLNLMEII | |
| | NDKDLGYAQM IEEATSCPED GKFTYEEVER LAGSPALKRG IWQSLQIVEE | |
| | ITKVMKCRPK YIYIEFERSE EAKERTESKI KKLENVYKDL DEQTKKEYKS | |
| | VLEELKGFDN TKKISSDSLF LYFTQLGKCM YSGKKLDIDS LDKYQIDHIV | |
| | PQSLVKDDSF DNRVLVVPSE NQRKLDDLVV PFDIRDKMYR FWKLLFDHEL | |
| | ISPKKFYSLI KTEYTERDEE RFINRQLVET RQITKNVTQI IEDHYSTTKV | |
| | AAIRANLSHE FRVKNHIYKN RDINDYHHAH DAYIVALIGG FMRDRYPNMH | |
| | DSKAVYSEYM KMFRKNKNDQ KRWKDGFVIN SMNYPYEVDG KLIWNPDLIN | |
| | EIKKCFYYKD CYCTTKLDQK SGQLFNLTVL SNDAHADKGV TKAVVPVNKN | |
| | RSDVHKYGGF SGLQYTIVAI EGQKKKGKKT ELVKKISGVP LHLKAASINE | |
| | KINYIEEKEG LSDVRIIKDN IPVNQMIEMD GGEYLLTSPT EYVNARQLVL | |
| | NEKQCALIAD IYNAIYKQDY DNLDDILMIQ LYIELTNKMK VLYPAYRGIA | |
| | EKFESMNENY VVISKEEKAN IIKQMLIVMH RGPQNGNIVY DDFKISDRIG | |
| | RLKTKNHNLN NIVFISQSPT GIYTKKYKL | |
| Mycoplasma | MLRLYCANNL VLNNVQNLWK YLLLLIFDKK IIFLFKIKVI LIRRYMENNN | (SEQ |
| synoviae 53 | KEKIVIGFDL GVASVGWSIV NAETKEVIDL GVRLFSEPEK ADYRRAKRTT | ID |
| AOL40776.1 | RRLLRRKKFK REKFHKLILK NAEIFGLQSR NEILNVYKDQ SSKYRNILKL | NO: |
| | KINALKEEIK PSELVWILRD YLQNRGYFYK NEKLTDEFVS NSFPSKKLHE | 69) |
| | HYEKYGFFRG SVKLDNKLDN KKDKAKEKDE EEESDAKKES EELIFSNKQW | |
| | INEIVKVFEN QSYLTESFKE EYLKLFNYVR PFNKGPGSKN SRTAYGVFST | |
| | DIDPETNKFK DYSNIWDKTI GKCSLFEEEI RAPKNLPSAL IFNLQNEICT | |
| | IKNEFTEFKN WWLNAEQKSE ILKFVFTELF NWKDKKYSDK KFNKNLQDKI | |
| | KKYLLNFALE NFNLNEEILK NRDLENDTVL GLKGVKYYEK SNATADAALE | |
| | FSSLKPLYVF IKFLKEKKLD LNYLLGLENT EILYFLDSIY LAISYSSDLK | |
| | ERNEWFKKLL KELYPKIKNN NLEIIENVED IFEITDQEKF ESFSKTHSLS | |
| | REAFNHIIPL LLSNNEGKNY ESLKHSNEEL KKRTEKAELK AQQNQKYLKD | |
| | NFLKEALVPL SVKTSVLQAI KIFNQIIKNF GKKYEISQVV IEMARELTKP | |
| | NLEKLLNNAT NSNIKILKEK LDQTEKFDDF TKKKFIDKIE NSVVFRNKLF | |
| | LWFEQDRKDP YTQLDIKINE IEDETEIDHV IPYSKSADDS WFNKLLVKKS | |
| | TNQLKKNKTV WEYYQNESDP EAKWNKFVAW AKRIYLVQKS DKESKDNSEK | |
| | NSIFKNKKPN LKFKNITKKL FDPYKDLGFL ARNLNDTRYA TKVFRDQLNN | |
| | YSKHHSKDDE NKLFKVVCMN GSITSFLRKS MWRKNEEQVY RFNFWKKDRD | |
| | QFFHHAVDAS IIAIFSLLTK TLYNKLRVYE SYDVQRREDG VYLINKETGE | |
| | VKKADKDYWK DQHNFLKIRE NAIEIKNVLN NVDFQNQVRY SRKANTKLNT | |
| | QLFNETLYGV KEFENNFYKL EKVNLFSRKD LRKFILEDLN EESEKNKKNE | |
| | NGSRKRILTE KYIVDEILQI LENEEFKDSK SDINALNKYM DSLPSKFSEF | |
| | FSQDFINKCK KENSLILTFD AIKHNDPKKV IKIKNLKFFR EDATLKNKQA | |
| | VHKDSKNQIK SFYESYKCVG FIWLKNKNDL EESIFVPINS RVIHFGDKDK | |
| | DIFDFDSYNK EKLLNEINLK RPENKKFNSI NEIEFVKFVK PGALLLNFEN | |
| | QQIYYISTLE SSSLRAKIKLLNKMDKGKAVS MKKITNPDEY KIIEHVNPL | |
| | GINLNWTKKL ENNN | |
| Flavobacterium | MAKILGLDLG TNSIGWAVVE RENIDFSLID KGVRIFSEGV KSEKGIESSR | (SEQ |
| branchiophilum | AAERTGYRSA RKIKYRRKLR KYETLKVLSL NRMCPLSIEE VEEWKKSGFK | ID |
| FL-15 | DYPLNPEFLK WLSTDEESNV NPYFFRDRAS KHKVSLFELG RAFYHIAQRR | NO: |
| WP_014084151.1 | GFLSNRLDQS AEGILEEHCP KIEAIVEDLI SIDEISTNIT DYFFETGILD | 70) |
| | SNEKNGYAKD LDEGDKKLVS LYKSLLAILK KNESDFENCK SEIIERLNKK | |
| | DVLGKVKGKI KDISQAMLDG NYKTLGQYFY SLYSKEKIRN QYTSREEHYL | |
| | SEFITICKVQ GIDQINEEEK INEKKFDGLA KDLYKAIFFQ RPLKSQKGLI | |
| | GKCSFEKSKS RCAISHPDFE EYRMWTYLNT IKIGTQSDKK LRFLTQDEKL | |
| | KLVPKFYRKN DFNFDVLAKE LIEKGSSFGF YKSSKKNDFF YWFNYKPTDT | |
| | VAACQVAASL KNAIGEDWKT KSFKYQTINS NKEQVSRTVD YKDLWHLLTV | |
| | ATSDVYLYEF AIDKLGLDEK NAKAFSKTKL KKDFASLSLS AINKILPYLK | |
| | EGLLYSHAVF VANIENIVDE NIWKDEKQRD YIKTQISEII ENYTLEKSRF | |
| | EIINGLLKEY KSENEDGKRV YYSKEAEQSF ENDLKKKLVL FYKSNEIENK | |
| | EQQETIFNEL LPIFIQQLKD YEFIKIQRLD QKVLIFLKGK NETGQIFCTE | |
| | EKGTAEEKEK KIKNRLKKLY HPSDIEKFKK KIIKDEFGNE KIVLGSPLTP | |
| | SIKNPMAMRA LHQLRKVLNA LILEGQIDEK TIIHIEMARE LNDANKRKGI | |
| | QDYQNDNKKF REDAIKEIKK LYFEDCKKEV EPTEDDILRY QLWMEQNRSE | |
| | IYEEGKNISI CDIIGSNPAY DIEHTIPRSR SQDNSQMNKT LCSQRFNREV | |
| | KKQSMPIELN NHLEILPRIA HWKEEADNLT REIEIISRSI KAAATKEIKD | |
| | KKIRRRHYLT LKRDYLQGKY DRFIWEEPKV GFKNSQIPDT GIITKYAQAY | |
| | LKSYFKKVES VKGGMVAEFR KIWGIQESFI DENGMKHYKV KDRSKHTHHT | |
| | IDAITIACMT KEKYDVLAHA WTLEDQQNKK EARSIIEASK PWKTFKEDLL | |
| | KIEEEILVSH YTPDNVKKQA KKIVRVRGKK QFVAEVERDV NGKAVPKKAA | |
| | SGKTIYKLDG EGKKLPRLQQ GDTIRGSLHQ DSIYGAIKNP LNTDEIKYVI | |
| | RKDLESIKGS DVESIVDEVV KEKIKEAIAN KVLLLSSNAQ QKNKLVGTVW | |
| | MNEEKRIAIN KVRIYANSVK NPLHIKEHSL LSKSKHVHKQ KVYGQNDENY | |
| | AMAIYELDGK RDFELINIFN LAKLIKQGQG FYPLHKKKEI KGKIVFVPIE | |
| | KRNKRDVVLK RGQQVVFYDK EVENPKDISE IVDFKGRIYI IEGLSIQRIV | |
| | RPSGKVDEYG VIMLRYFKEA RKADDIKQDN FKPDGVFKLG ENKPTRKMNH | |
| | NQFTAFVEGI DFKVLPSGKF EKI | |
| Eubacterium | MENKQYYIGL DVGTNSVGWA VTDTSYNLLR AKGKDMWGAR LFEKANTAAE | (SEQ |
| yurii subsp. | RRTKRTSRRR SEREKARKAM LKELFADEIN RVDPSFFIRL EESKFFLDDR | ID |
| margaretiae | SENNRQRYTL FNDATFTDKD YYEKYKTIFH LRSALINSDE KFDVRLVFLA | NO: |
| ATCC 43715 | ILNLFSHRGH FLNASLKGDG DIQGMDVFYN DLVESCEYFE IELPRITNID | 71) |
| EFM38267.1 | NFEKILSQKG KSRTKILEEL SEELSISKKD KSKYNLIKLI SGLEASVVEL | |
| | YNIEDIQDEN KKIKIGFRES DYEESSLKVK EIIGDEYFDL VERAKSVHDM | |
| | GLLSNIIGNS KYLCEARVEA YENHHKDLLK IKELLKKYDK KAYNDMFRKM | |
| | TDKNYSAYVG SVNSNIAKER RSVDKRKIED LYKYIEDTAL KNIPDDNKDK | |
| | IEILEKIKLG EFLKKQLTAS NGVIPNQLQS RELRAILKKA ENYLPFLKEK | |
| | GEKNLTVSEM IIQLFEFQIP YYVGPLDKNP KKDNKANSWA KIKQGGRILP | |
| | WNFEDKVDVK GSRKEFIEKM VRKCTYISDE HTLPKQSLLY EKFMVLNEIN | |
| | NIKIDGEKIS VEAKQKIYND LFVKGKKVSQ KDIKKELISL NIMDKDSVLS | |
| | GTDTVCNAYL SSIGKFTGVF KEEINKQSIV DMIEDIIFLK TVYGDEKRFV | |
| | KEEIVEKYGD EIDKDKIKRI LGFKFSNWGN LSKSFLELEG ADVGTGEVRS | |
| | IIQSLWETNF NLMELLSSRF TYMDELEKRV KKLEKPLSEW TIEDLDDMYL | |
| | SSPVKRMIWQ SMKIVDEIQT VIGYAPKRIF VEMTRSEGEK VRTKSRKDRL | |
| | KELYNGIKED SKQWVKELDS KDESYFRSKK MYLYYLQKGR CMYSGEVIEL | |
| | DKLMDDNLYD IDHIYPRSFV KDDSLDNLVL VKKEINNRKQ NDPITPQIQA | |
| | SCQGFWKILH DQGFMSNEKY SRLTRKTQEF SDEEKLSFIN RQIVETGQAT | |
| | KCMAQILQKS MGEDVDVVFS KARLVSEFRH KFELFKSRLI NDFHHANDAY | |
| | LNIVVGNSYF VKFTRNPANF IKDARKNPDN PVYKYHMDRF FERDVKSKSE | |
| | VAWIGQSEGN SGTIVIVKKT MAKNSPLITK KVEEGHGSIT KETIVGVKEI | |
| | KFGRNKVEKA DKTPKKPNLQ AYRPIKTSDE RLCNILRYGG RTSISISGYC | |
| | LVEYVKKRKT IRSLEAIPVY LGRKDSLSEE KLLNYFRYNL NDGGKDSVSD | |
| | IRLCLPFIST NSLVKIDGYL YYLGGKNDDR IQLYNAYQLK MKKEEVEYIR | |
| | KIEKAVSMSK FDEIDREKNP VLTEEKNIEL YNKIQDKFEN TVFSKRMSLV | |
| | KYNKKDLSFG DFLKNKKSKF EEIDLEKQCK VLYNIIFNLS NLKEVDLSDI | |
| | GGSKSTGKCR CKKNITNYKE FKLIQQSITG LYSCEKDLMT I | |
| Acidovorax | MAQHVFGLDI GIASVGWAIL GEQRIIDLGV RCFDKAETAK EGDPLNLTRR | (SEQ |
| ebreus | QARLLRRRLY RRAWRLTQLS RLLKRKGLIA DAKLFAKAPS YGDSAWELRR | ID |
| WP_012655176.1 | QGLDRLLTPL EWARVIYHQC KHRGFHWTSK AEEAKADSDA EGGRVKQGLA | NO: |
| | HTKALMQAKN YRSAAEMVLA EFPDAQRNKR GQYDKALSRV LLGEELALLF | 72) |
| | ATQRRLGNPH ASDFFEKLIL GDGDRKSGLF WQQKPALSGA DLLKMLGKCT | |
| | FEKGEYRAPK ASFSVERHVW LTRLNNLRIV VDGRSRPLNE AERQAALLLP | |
| | YQTETSKYKT LKNAFIKAGL WGDGVRFGGL AYPSQAQIDA EKTKDPEDQF | |
| | LVKLPAWHEL RKAFKAAGHE ALWQQISTPA LDGDPTLLDQ IATVLSVYKD | |
| | GAEVVQQLRQ LALPEPAASI AVLEKISFDK FSSLSLKALR RIVPLMQSGL | |
| | RYDEAVAQIP EYGHHSQRIE PGAAKHLYLP PFYEAQRKYA GKGDHIGSMQ | |
| | FRDDADIPRN PVVLRALNQA RKVVNALIRE YGSPIAVNIE MARDLSRPLD | |
| | ERNKVKRAQE EFRDRNDRAR SEFERDFGYK PKAAAFEKWM LYREQLGQCA | |
| | YSQQPLDIQR VLDDHNYAQV DHALPYSRSY DDSKNNKVLV LTHENQNKGN | |
| | RTAFEYLTSF PDGEDGERWR TFVAWVQGNK AYRMAKRNRL LRKNYGVDES | |
| | KGFIDRNLND TRYICKFFKN YVEEHLQLAA RADGDTARRC VVVNGQLTAF | |
| | LRARWGLTKV RGDSDRHHAL DAAVVAACTH GMVKALADYS RRKEISFLQE | |
| | GFPDPETGEI LNPAAFDRAR QHFPEPWTHF AHELKARLFT DDLAALREDM | |
| | QRLGSYTTED LGRLRTLFVS RAPQRRSGGA VHKETIYAQP ESLKQQGGVI | |
| | EKILLTSLKL QDFDKLLNPE SNDHFVEPHR NERLYAAIRQ RLEQFGGRAD | |
| | KAFGPDNLFH KPDKNNQPTG PVVRSIKLVR GKQTGIPIRG GLAKNDSMLR | |
| | VDIFTKAGKF HLVPVYVHHR VTGLPNRAIV AFKDEDEWTL IDESFAFLFS | |
| | VYPNDYVKVT LKKEQQSGYY SGADRSTGAM NLWAHDRAAS VGKDGLIRGI | |
| | GVKTALSVEK FNVDVLGRIY LAPPETRSGL A | |
| Porphyromonas | MLMSKHVLGL DLGVGSIGWC LIALDAQGDP AEILGMGSRV VPLNNATKAI | (SEQ |
| sp. oral taxon | EAFNAGAAFT ASQERTARRT MRRGFARYQL RRYRLRRELE KVGMLPDAAL | ID |
| 279 str. F0450 | IQLPLLELWE LRERAATAGR RLTLPELGRV LCHINQKRGY RHVKSDAAAI | NO: |
| WP_009433518.1 | VGDEGEKKKD SNSAYLAGIR ANDEKLQAEH KTVGQYFAEQ LRQNQSESPT | 73) |
| | GGISYRIKDQ IFSRQCYIDE YDQIMAVQRV HYPDILTDEF IRMLRDEVIF | |
| | MQRPLKSCKH LVSLCEFEKQ ERVMRVQQDD GKGGWQLVER RVKFGPKVAP | |
| | KSSPLFQLCC IYEAVNNIRL TRPNGSPCDI TPEERAKIVA HLQSSASLSF | |
| | AALKKLLKEK ALIADQLTSK SGLKGNSTRV ALASALQPYP QYHHLLDMEL | |
| | ETRMMTVQLT DEETGEVTER EVAVVTDSYV RKPLYRLWHI LYSIEEREAM | |
| | RRALITQLGM KEEDLDGGLL DQLYRLDFVK PGYGNKSAKF ICKLLPQLQQ | |
| | GLGYSEACAA VGYRHSNSPT SEEITERTLL EKIPLLQRNE LRQPLVEKIL | |
| | NQMINLVNAL KAEYGIDEVR VELARELKMS REERERMARN NKDREERNKG | |
| | VAAKIRECGL YPTKPRIQKY MLWKEAGRQC LYCGRSIEEE QCLREGGMEV | |
| | EHIIPKSVLY DDSYGNKTCA CRRCNKEKGN RTALEYIRAK GREAEYMKRI | |
| | NDLLKEKKIS YSKHQRLRWL KEDIPSDFLE RQLRLTQYIS RQAMAILQQG | |
| | IRRVSASEGG VTARLRSLWG YGKILHTLNL DRYDSMGETE RVSREGEATE | |
| | ELHITNWSKR MDHRHHAIDA LVVACTRQSY IQRLNRLSSE FGREDKKKED | |
| | QEAQEQQATE TGRLSNLERW LTQRPHFSVR TVSDKVAEIL ISYRPGQRVV | |
| | TRGRNIYRKK MADGREVSCV QRGVLVPRGE LMEASFYGKI LSQGRVRIVK | |
| | RYPLHDLKGE VVDPHLRELI TTYNQELKSR EKGAPIPPLC LDKDKKQEVR | |
| | SVRCYAKTLS LDKAIPMCFD EKGEPTAFVK SASNHHLALY RTPKGKLVES | |
| | IVTFWDAVDR ARYGIPLVIT HPREVMEQVL QRGDIPEQVL SLLPPSDWVF | |
| | VDSLQQDEMV VIGLSDEELQ RALEAQNYRK ISEHLYRVQK MSSSYYVFRY | |
| | HLETSVADDK NTSGRIPKFH RVQSLKAYEE RNIRKVRVDL LGRISLL | |
| Mycoplasma | MHNKKNITIG FDLGIASIGW AIIDSTTSKI LDWGTRTFEE RKTANERRAF | (SEQ |
| ovipneumoniae | RSTRRNIRRK AYRNQRFINL ILKYKDLFEL KNISDIQRAN KKDTENYEKI | ID |
| SC01 | ISFFTEIYKK CAAKHSNILE VKVKALDSKI EKLDLIWILH DYLENRGFFY | NO: |
| WP_010320922.1 | DLEEENVADK YEGIEHPSIL LYDFFKKNGF FKSNSSIPKD LGGYSFSNLQ | 74) |
| | WVNEIKKLFE VQEINPEFSE KFLNLFTSVR DYAKGPGSEH SASEYGIFQK | |
| | DEKGKVFKKY DNIWDKTIGK CSFFVEENRS PVNYPSYEIF NLLNQLINLS | |
| | TDLKTTNKKI WQLSSNDRNE LLDELLKVKE KAKIISISLK KNEIKKIILK | |
| | DFGFEKSDID DQDTIEGRKI IKEEPTTKLE VTKHLLATIY SHSSDSNWIN | |
| | INNILEFLPY LDAICIILDR EKSRGQDEVL KKLTEKNIFE VLKIDREKQL | |
| | DFVKSIFSNT KFNFKKIGNF SLKAIREFLP KMFEQNKNSE YLKWKDEEIR | |
| | RKWEEQKSKL GKTDKKTKYL NPRIFQDEII SPGTKNTFEQ AVLVLNQIIK | |
| | KYSKENIIDA IIIESPREKN DKKTIEEIKK RNKKGKGKTL EKLFQILNLE | |
| | NKGYKLSDLE TKPAKLLDRL RFYHQQDGID LYTLDKINID QLINGSQKYE | |
| | IEHIIPYSMS YDNSQANKIL TEKAENLKKG KLIASEYIKR NGDEFYNKYY | |
| | EKAKELFINK YKKNKKLDSY VDLDEDSAKN RFRFLTLQDY DEFQVEFLAR | |
| | NLNDTRYSTK LFYHALVEHF ENNEFFTYID ENSSKHKVKI STIKGHVTKY | |
| | FRAKPVQKNN GPNENLNNNK PEKIEKNREN NEHHAVDAAI VAIIGNKNPQ | |
| | IANLLTLADN KTDKKFLLHD ENYKENIETG ELVKIPKFEV DKLAKVEDLK | |
| | KIIQEKYEEA KKHTAIKFSR KTRTILNGGL SDETLYGFKY DEKEDKYFKI | |
| | IKKKLVTSKN EELKKYFENP FGKKADGKSE YTVLMAQSHL SEFNKLKEIF | |
| | EKYNGFSNKT GNAFVEYMND LALKEPTLKA EIESAKSVEK LLYYNFKPSD | |
| | QFTYHDNINN KSFKRFYKNI RIIEYKSIPI KFKILSKHDG GKSFKDTLFS | |
| | LYSLVYKVYE NGKESYKSIP VTSQMRNFGI DEFDFLDENL YNKEKLDIYK | |
| | SDFAKPIPVN CKPVFVLKKG SILKKKSLDI DDFKETKETE EGNYYFISTI | |
| | SKRFNRDTAY GLKPLKLSVV KPVAEPSTNP IFKEYIPIHL DELGNEYPVK | |
| | IKEHTDDEKL MCTIK | |
| Wolinella | MLVSPISVDL GGKNTGFFSF TDSLDNSQSG TVIYDESFVL SQVGRRSKRH | (SEQ |
| succinogenes | SKRNNLRNKL VKRLFLLILQ EHHGLSIDVL PDEIRGLFNK RGYTYAGFEL | ID |
| WP_011139431.1 | DEKKKDALES DTLKEFLSEK LQSIDRDSDV EDFLNQIASN AESFKDYKKG | NO: |
| | FEAVFASATH SPNKKLELKD ELKSEYGENA KELLAGLRVT KEILDEFDKQ | 75) |
| | ENQGNLPRAK YFEELGEYIA TNEKVKSFFD SNSLKLTDMT KLIGNISNYQ | |
| | LKELRRYFND KEMEKGDIWI PNKLHKITER FVRSWHPKND ADRQRRAELM | |
| | KDLKSKEIME LLTTTEPVMT IPPYDDMNNR GAVKCQTLRL NEEYLDKHLP | |
| | NWRDIAKRLN HGKFNDDLAD STVKGYSEDS TLLHRLLDTS KEIDIYELRG | |
| | KKPNELLVKT LGQSDANRLY GFAQNYYELI RQKVRAGIWV PVKNKDDSLN | |
| | LEDNSNMLKR CNHNPPHKKN QIHNLVAGIL GVKLDEAKFA EFEKELWSAK | |
| | VGNKKLSAYC KNIEELRKTH GNTFKIDIEE LRKKDPAELS KEEKAKLRLT | |
| | DDVILNEWSQ KIANFFDIDD KHRQRFNNLF SMAQLHTVID TPRSGFSSTC | |
| | KRCTAENRFR SETAFYNDET GEFHKKATAT CQRLPADTQR PFSGKIERYI | |
| | DKLGYELAKI KAKELEGMEA KEIKVPIILE QNAFEYEESL RKSKTGSNDR | |
| | VINSKKDRDG KKLAKAKENA EDRLKDKDKR IKAFSSGICP YCGDTIGDDG | |
| | EIDHILPRSH TLKIYGTVFN PEGNLIYVHQ KCNQAKADSI YKLSDIKAGV | |
| | SAQWIEEQVA NIKGYKTFSV LSAEQQKAFR YALFLQNDNE AYKKVVDWLR | |
| | TDQSARVNGT QKYLAKKIQE KLTKMLPNKH LSFEFILADA TEVSELRRQY | |
| | ARQNPLLAKA EKQAPSSHAI DAVMAFVARY QKVFKDGTPP NADEVAKLAM | |
| | LDSWNPASNE PLTKGLSTNQ KIEKMIKSGD YGQKNMREVF GKSIFGENAI | |
| | GERYKPIVVQ EGGYYIGYPA TVKKGYELKN CKVVTSKNDI AKLEKIIKNQ | |
| | DLISLKENQY IKIFSINKQT ISELSNRYFN MNYKNLVERD KEIVGLLEFI | |
| | VENCRYYTKK VDVKFAPKYI HETKYPFYDD WRRFDEAWRY LQENQNKTSS | |
| | KDRFVIDKSS LNEYYQPDKN EYKLDVDTQP IWDDFCRWYF LDRYKTANDK | |
| | KSIRIKARKT FSLLAESGVQ GKVFRAKRKI PTGYAYQALP MDNNVIAGDY | |
| | ANILLEANSK TLSLVPKSGI SIEKQLDKKL DVIKKTDVRG LAIDNNSFFN | |
| | ADFDTHGIRL IVENTSVKVG NFPISAIDKS AKRMIFRALF EKEKGKRKKK | |
| | TTISFKESGP VQDYLKVFLK KIVKIQLRTD GSISNIVVRK NAADFTLSFR | |
| | SEHIQKLLK | |
| Streptococcus | MKKPYSIGLD IGTNSVGWAV VTDDYKVPAK KMKVLGNTDK SHIEKNLLGA | (SEQ |
| mutans UA159 | LLFDSGNTAE DRRLKRTARR RYTRRRNRIL YLQEIFSEEM GKVDDSFFHR | ID |
| WP_002263549.1 | LEDSFLVTED KRGERHPIFG NLEEEVKYHE NFPTIYHLRQ YLADNPEKVD | NO: |
| | LRLVYLALAH IIKFRGHFLI EGKFDTRNND VQRLFQEFLA VYDNTFENSS | 76) |
| | LQEQNVQVEE ILTDKISKSA KKDRVLKLFP NEKSNGRFAE FLKLIVGNQA | |
| | DFKKHFELEE KAPLQFSKDT YEEELEVLLA QIGDNYAELF LSAKKLYDSI | |
| | LLSGILTVTD VGTKAPLSAS MIQRYNEHQM DLAQLKQFIR QKLSDKYNEV | |
| | FSDVSKDGYA GYIDGKTNQE AFYKYLKGLL NKIEGSGYFL DKIEREDFLR | |
| | KQRTFDNGSI PHQIHLQEMR AIIRRQAEFY PFLADNQDRI EKLLTFRIPY | |
| | YVGPLARGKS DFAWLSRKSA DKITPWNFDE IVDKESSAEA FINRMTNYDL | |
| | YLPNQKVLPK HSLLYEKFTV YNELTKVKYK TEQGKTAFFD ANMKQEIFDG | |
| | VFKVYRKVTK DKLMDFLEKE FDEFRIVDLT GLDKENKVEN ASYGTYHDLC | |
| | KILDKDFLDN SKNEKILEDI VLTLTLFEDR EMIRKRLENY SDLLTKEQVK | |
| | KLERRHYTGW GRLSAELIHG IRNKESRKTI LDYLIDDGNS NRNFMQLIND | |
| | DALSFKEEIA KAQVIGETDN LNQVVSDIAG SPAIKKGILQ SLKIVDELVK | |
| | IMGHQPENIV VEMARENQFT NQGRRNSQQR LKGLTDSIKE FGSQILKEHP | |
| | VENSQLQNDR LFLYYLQNGR DMYTGEELDI DYLSQYDIDH IIPQAFIKDN | |
| | SIDNRVLTSS KENRGKSDDV PSKDVVRKMK SYWSKLLSAK LITQRKFDNL | |
| | TKAERGGLTD DDKAGFIKRQ LVETRQITKH VARILDERFN TETDENNKKI | |
| | RQVKIVTLKS NLVSNFRKEF ELYKVREIND YHHAHDAYLN AVIGKALLGV | |
| | YPQLEPEFVY GDYPHFHGHK ENKATAKKFF YSNIMNFFKK DDVRTDKNGE | |
| | IIWKKDEHIS NIKKVLSYPQ VNIVKKVEEQ TGGFSKESIL PKGNSDKLIP | |
| | RKTKKFYWDT KKYGGFDSPI VAYSILVIAD IEKGKSKKLK TVKALVGVTI | |
| | MEKMTFERDP VAFLERKGYR NVQEENIIKL PKYSLFKLEN GRKRLLASAR | |
| | ELQKGNEIVL PNHLGTLLYH AKNIHKVDEP KHLDYVDKHK DEFKELLDVV | |
| | SNFSKKYTLA EGNLEKIKEL YAQNNGEDLK ELASSFINLL TFTAIGAPAT | |
| | FKFFDKNIDR KRYTSTTEIL NATLIHQSIT GLYETRIDLN KLGGD | |
| Prevotella | MNKRILGLDT GTNSLGWAVV DWDEHAQSYE LIKYGDVIFQ EGVKIEKGIE | (SEQ |
| timonensis | SSKAAERSGY KAIRKQYFRR RLRKIQVLKV LVKYHLCPYL SDDDLRQWHL | ID |
| CRIS 5C-B1 | QKQYPKSDEL MLWQRTSDEE GKNPYYDRHR CLHEKLDLTV EADRYTLGRA | NO: |
| WP_008122718.1 | LYHLTQRRGF LSNRLDTSAD NKEDGVVKSG ISQLSTEMEE AGCEYLGDYF | 77) |
| | YKLYDAQGNK VRIRQRYTDR NKHYQHEFDA ICEKQELSSE LIEDLQRAIF | |
| | FQLPLKSQRH GVGRCTFERG KPRCADSHPD YEEFRMLCFV NNIQVKGPHD | |
| | LELRPLTYEE REKIEPLFFR KSKPNFDFED IAKALAGKKN YAWIHDKEER | |
| | AYKFNYRMTQ GVPGCPTIAQ LKSIFGDDWK TGIAETYTLI QKKNGSKSLQ | |
| | EMVDDVWNVL YSFSSVEKLK EFAHHKLQLD EESAEKFAKI KLSHSFAALS | |
| | LKAIRKFLPF LRKGMYYTHA SFFANIPTIV GKEIWNKEQN RKYIMENVGE | |
| | LVFNYQPKHR EVQGTIEMLI KDFLANNFEL PAGATDKLYH PSMIETYPNA | |
| | QRNEFGILQL GSPRTNAIRN PMAMRSLHIL RRVVNQLLKE SIIDENTEVH | |
| | VEYARELNDA NKRRAIADRQ KEQDKQHKKY GDEIRKLYKE ETGKDIEPTQ | |
| | TDVLKFQLWE EQNHHCLYTG EQIGITDFIG SNPKFDIEHT IPQSVGGDST | |
| | QMNLTLCDNR FNREVKKAKL PTELANHEEI LTRIEPWKNK YEQLVKERDK | |
| | QRTFAGMDKA VKDIRIQKRH KLQMEIDYWR GKYERFTMTE VPEGFSRRQG | |
| | TGIGLISRYA GLYLKSLFHQ ADSRNKSNVY VVKGVATAEF RKMWGLQSEY | |
| | EKKCRDNHSH HCMDAITIAC IGKREYDLMA EYYRMEETFK QGRGSKPKFS | |
| | KPWATFTEDV LNIYKNLLVV HDTPNNMPKH TKKYVQTSIG KVLAQGDTAR | |
| | GSLHLDTYYG AIERDGEIRY VVRRPLSSFT KPEELENIVD ETVKRTIKEA | |
| | IADKNFKQAI AEPIYMNEEK GILIKKVRCF AKSVKQPINI RQHRDLSKKE | |
| | YKQQYHVMNE NNYLLAIYEG LVKNKVVREF EIVSYIEAAK YYKRSQDRNI | |
| | FSSIVPTHST KYGLPLKTKL LMGQLVLMFE ENPDEIQVDN TKDLVKRLYK | |
| | VVGIEKDGRI KFKYHQEARK EGLPIFSTPY KNNDDYAPIF RQSINNINIL | |
| | VDGIDFTIDI LGKVTLKE | |
| Clostridium | MKYTLGLDVG IASVGWAVID KDNNKIIDLG VRCFDKAEES KTGESLATAR | (SEQ |
| cellulolyticum | RIARGMRRRI SRRSQRLRLV KKLFVQYEII KDSSEFNRIF DTSRDGWKDP | ID |
| H10 | WELRYNALSR ILKPYELVQV LTHITKRRGF KSNRKEDLST TKEGVVITSI | NO: |
| ACL77411.1 | KNNSEMLRTK NYRTIGEMIF METPENSNKR NKVDEYIHTI AREDLLNEIK | 78) |
| | YIFSIQRKLG SPFVTEKLEH DFLNIWEFQR PFASGDSILS KVGKCTLLKE | |
| | ELRAPTSCYT SEYFGLLQSI NNLVLVEDNN TLTLNNDQRA KIIEYAHFKN | |
| | EIKYSEIRKL LDIEPEILFK AHNLTHKNPS GNNESKKFYE MKSYHKLKST | |
| | LPTDIWGKLH SNKESLDNLF YCLTVYKNDN EIKDYLQANN LDYLIEYIAK | |
| | LPTFNKFKHL SLVAMKRIIP FMEKGYKYSD ACNMAELDFT GSSKLEKCNK | |
| | LTVEPIIENV TNPVVIRALT QARKVINAII QKYGLPYMVN IELAREAGMT | |
| | RQDRDNLKKE HENNRKAREK ISDLIRQNGR VASGLDILKW RLWEDQGGRC | |
| | AYSGKPIPVC DLLNDSLTQI DHIYPYSRSM DDSYMNKVLV LTDENQNKRS | |
| | YTPYEVWGST EKWEDFEARI YSMHLPQSKE KRLLNRNFIT KDLDSFISRN | |
| | LNDTRYISRF LKNYIESYLQ FSNDSPKSCV VCVNGQCTAQ LRSRWGLNKN | |
| | REESDLHHAL DAAVIACADR KIIKEITNYY NERENHNYKV KYPLPWHSFR | |
| | QDLMETLAGV FISRAPRRKI TGPAHDETIR SPKHENKGLT SVKIPLTTVT | |
| | LEKLETMVKN TKGGISDKAV YNVLKNRLIE HNNKPLKAFA EKIYKPLKNG | |
| | TNGAIIRSIR VETPSYTGVF RNEGKGISDN SLMVRVDVFK KKDKYYLVPI | |
| | YVAHMIKKEL PSKAIVPLKP ESQWELIDST HEFLFSLYQN DYLVIKTKKG | |
| | ITEGYYRSCH RGTGSLSLMP HFANNKNVKI DIGVRTAISI EKYNVDILGN | |
| | KSIVKGEPRR GMEKYNSFKS N | |
| Francisella | MNFKILPIAI DLGVKNTGVF SAFYQKGTSL ERLDNKNGKV YELSKDSYTL | (SEQ |
| tularensis | LMNNRTARRH QRRGIDRKQL VKRLFKLIWT EQLNLEWDKD TQQAISFLEN | ID |
| subsp. | RRGFSFITDG YSPEYLNIVP EQVKAILMDI FDDYNGEDDL DSYLKLATEQ | NO: |
| novicida U112 | ESKISEIYNK LMQKILEFKL MKLCTDIKDD KVSTKTLKEI TSYEFELLAD | 79) |
| WP_003038941.1 | YLANYSESLK TQKFSYTDKQ GNLKELSYYH HDKYNIQEFL KRHATINDRI | |
| | LDTLLTDDLD IWNFNFEKFD FDKNEEKLQN QEDKDHIQAH LHHFVFAVNK | |
| | IKSEMASGGR HRSQYFQEIT NVLDENNHQE GYLKNFCENL HNKKYSNLSV | |
| | KNLVNLIGNL SNLELKPLRK YFNDKIHAKA DHWDEQKFTE TYCHWILGEW | |
| | RVGVKDQDKK DGAKYSYKDL CNELKQKVTK AGLVDFLLEL DPCRTIPPYL | |
| | DNNNRKPPKC QSLILNPKFL DNQYPNWQQY LQELKKLQSI QNYLDSFETD | |
| | LKVLKSSKDQ PYFVEYKSSN QQIASGQRDY KDLDARILQF IFDRVKASDE | |
| | LLLNEIYFQA KKLKQKASSE LEKLESSKKL DEVIANSQLS QILKSQHING | |
| | IFEQGTFLHL VCKYYKQRQR ARDSRLYIMP EYRYDKKLHK YNNTGRFDDD | |
| | NQLLTYCNHK PRQKRYQLLN DLAGVLQVSP NFLKDKIGSD DDLFISKWLV | |
| | EHIRGFKKAC EDSLKIQKDN RGLLNHKINI ARNTKGKCEK EIFNLICKIE | |
| | GSEDKKGNYK HGLAYELGVL LFGEPNEASK PEFDRKIKKF NSIYSFAQIQ | |
| | QIAFAERKGN ANTCAVCSAD NAHRMQQIKI TEPVEDNKDK IILSAKAQRL | |
| | PAIPTRIVDG AVKKMATILA KNIVDDNWQN IKQVLSAKHQ LHIPIITESN | |
| | AFEFEPALAD VKGKSLKDRR KKALERISPE NIFKDKNNRI KEFAKGISAY | |
| | SGANLTDGDF DGAKEELDHI IPRSHKKYGT LNDEANLICV TRGDNKNKGN | |
| | RIFCLRDLAD NYKLKQFETT DDLEIEKKIA DTIWDANKKD FKFGNYRSFI | |
| | NLTPQEQKAF RHALFLADEN PIKQAVIRAI NNRNRTFVNG TQRYFAEVLA | |
| | NNIYLRAKKE NLNTDKISFD YFGIPTIGNG RGIAEIRQLY EKVDSDIQAY | |
| | AKGDKPQASY SHLIDAMLAF CIAADEHRND GSIGLEIDKN YSLYPLDKNT | |
| | GEVFTKDIFS QIKITDNEFS DKKLVRKKAI EGFNTHRQMT RDGIYAENYL | |
| | PILIHKELNE VRKGYTWKNS EEIKIFKGKK YDIQQLNNLV YCLKFVDKPI | |
| | SIDIQISTLE ELRNILTTNN IAATAEYYYI NLKTQKLHEY YIENYNTALG | |
| | YKKYSKEMEF LRSLAYRSER VKIKSIDDVK QVLDKDSNFI IGKITLPFKK | |
| | EWQRLYREWQ NTTIKDDYEF LKSFFNVKSI TKLHKKVRKD FSLPISTNEG | |
| | KFLVKRKTWD NNFIYQILND SDSRADGTKP FIPAFDISKN EIVEAIIDSF | |
| | TSKNIFWLPK NIELQKVDNK NIFAIDTSKW FEVETPSDLR DIGIATIQYK | |
| | IDNNSRPKVR VKLDYVIDDD SKINYFMNHS LLKSRYPDKV LEILKQSTII | |
| | EFESSGFNKT IKEMLGMKLA GIYNETSNN | |
| Azospirillum | MARPAFRAPR REHVNGWTPD PHRISKPFFI LVSWHLLSRV VIDSSSGCFP | (SEQ |
| sp. B510 | GTSRDHTDKF AEWECAVQPY RLSFDLGTNS IGWGLLNLDR QGKPREIRAL | ID |
| AOL40891.1 | GSRIFSDGRD PQDKASLAVA RRLARQMRRR RDRYLTRRTR LMGALVRFGL | NO: |
| | MPADPAARKR LEVAVDPYLA RERATRERLE PFEIGRALFH LNQRRGYKPV | 80) |
| | RTATKPDEEA GKVKEAVERL EAAIAAAGAP TLGAWFAWRK TRGETLRARL | |
| | AGKGKEAAYP FYPARRMLEA EFDTLWAEQA RHHPDLLTAE AREILRHRIF | |
| | HQRPLKPPPV GRCTLYPDDG RAPRALPSAQ RLRLFQELAS LRVIHLDLSE | |
| | RPLTPAERDR IVAFVQGRPP KAGRKPGKVQ KSVPFEKLRG LLELPPGTGF | |
| | SLESDKRPEL LGDETGARIA PAFGPGWTAL PLEEQDALVE LLLTEAEPER | |
| | AIAALTARWA LDEATAAKLA GATLPDFHGR YGRRAVAELL PVLERETRGD | |
| | PDGRVRPIRL DEAVKLLRGG KDHSDFSREG ALLDALPYYG AVLERHVAFG | |
| | TGNPADPEEK RVGRVANPTV HIALNQLRHL VNAILARHGR PEEIVIELAR | |
| | DLKRSAEDRR REDKRQADNQ KRNEERKRLI LSLGERPTPR NLLKLRLWEE | |
| | QGPVENRRCP YSGETISMRM LLSEQVDIDH ILPFSVSLDD SAANKVVCLR | |
| | EANRIKRNRS PWEAFGHDSE RWAGILARAE ALPKNKRWRF APDALEKLEG | |
| | EGGLRARHLN DTRHLSRLAV EYLRCVCPKV RVSPGRLTAL LRRRWGIDAI | |
| | LAEADGPPPE VPAETLDPSP AEKNRADHRH HALDAVVIGC IDRSMVQRVQ | |
| | LAAASAEREA AAREDNIRRV LEGFKEEPWD GFRAELERRA RTIVVSHRPE | |
| | HGIGGALHKE TAYGPVDPPE EGFNLVVRKP IDGLSKDEIN SVRDPRLRRA | |
| | LIDRLAIRRR DANDPATALA KAAEDLAAQP ASRGIRRVRV LKKESNPIRV | |
| | EHGGNPSGPR SGGPFHKLLL AGEVHHVDVA LRADGRRWVG HWVTLFEAHG | |
| | GRGADGAAAP PRLGDGERFL MRLHKGDCLK LEHKGRVRVM QVVKLEPSSN | |
| | SVVVVEPHQV KTDRSKHVKI SCDQLRARGA RRVTVDPLGR VRVHAPGARV | |
| | GIGGDAGRTA MEPAEDIS | |
| Peptoniphilus | MKNLKEYYIG LDIGTASVGW AVTDESYNIP KFNGKKMWGV RLFDDAKTAE | (SEQ |
| duerdenii ATCC | ERRTQRGSRR RLNRRKERIN LLQDLFATEI SKVDPNFFLR LDNSDLYRED | ID |
| BAA-1640 | KDEKLKSKYT LFNDKDFKDR DYHKKYPTIH HLIMDLIEDE GKKDIRLLYL | NO: |
| WP_008901059.1 | ACHYLLKNRG HFIFEGQKFD TKNSFDKSIN DLKIHLRDEY NIDLEFNNED | 81) |
| | LIEIITDTTL NKTNKKKELK NIVGDTKFLK AISAIMIGSS QKLVDLFEDG | |
| | EFEETTVKSV DFSTTAFDDK YSEYEEALGD TISLLNILKS IYDSSILENL | |
| | LKDADKSKDG NKYISKAFVK KFNKHGKDLK TLKRIIKKYL PSEYANIFRN | |
| | KSINDNYVAY TKSNITSNKR TKASKFTKQE DFYKFIKKHL DTIKETKLNS | |
| | SENEDLKLID EMLTDIEFKT FIPKLKSSDN GVIPYQLKLM ELKKILDNQS | |
| | KYYDFLNESD EYGTVKDKVE SIMEFRIPYY VGPLNPDSKY AWIKRENTKI | |
| | TPWNFKDIVD LDSSREEFID RLIGRCTYLK EEKVLPKASL IYNEFMVLNE | |
| | LNNLKLNEFL ITEEMKKAIF EELFKTKKKV TLKAVSNLLK KEFNLTGDIL | |
| | LSGTDGDFKQ GLNSYIDFKN IIGDKVDRDD YRIKIEEIIK LIVLYEDDKT | |
| | YLKKKIKSAY KNDFTDDEIK KIAALNYKDW GRLSKRFLTG IEGVDKTTGE | |
| | KGSIIYFMRE YNLNLMELMS GHYTFTEEVE KLNPVENREL CYEMVDELYL | |
| | SPSVKRMLWQ SLRVVDEIKR IIGKDPKKIF IEMARAKEAK NSRKESRKNK | |
| | LLEFYKFGKK AFINEIGEER YNYLLNEINS EEESKFRWDN LYLYYTQLGR | |
| | CMYSLEPIDL ADLKSNNIYD QDHIYPKSKI YDDSLENRVL VKKNLNHEKG | |
| | NQYPIPEKVL NKNAYGFWKI LFDKGLIGQK KYTRLTRRTP FEERELAEFI | |
| | ERQIVETRQA TKETANLLKN ICQDSEIVYS KAENASRFRQ EFDIIKCRTV | |
| | NDLHHMHDAY LNIVVGNVYN TKFTKNPLNF IKDKDNVRSY NLENMFKYDV | |
| | VRGSYTAWIA DDSEGNVKAA TIKKVKRELE GKNYRFTRMS YIGTGGLYDQ | |
| | NLMRKGKGQI PQKENTNKSN IEKYGGYNKA SSAYFALIES DGKAGRERTL | |
| | ETIPIMVYNQ EKYGNTEAVD KYLKDNLELQ DPKILKDKIK INSLIKLDGF | |
| | LYNIKGKTGD SLSIAGSVQL IVNKEEQKLI KKMDKFLVKK KDNKDIKVTS | |
| | FDNIKEEELI KLYKTLSDKL NNGIYSNKRN NQAKNISEAL DKFKEISIEE | |
| | KIDVLNQIIL LFQSYNNGCN LKSIGLSAKT GVVFIPKKLN YKECKLINQS | |
| | ITGLFENEVD LLNL | |
| Lactobacillus | MGYRIGLDVG ITSTGYAVLK TDKNGLPYKI LTLDSVIYPR AENPQTGASL | (SEQ |
| coryniformis | AEPRRIKRGL RRRTRRTKFR KQRTQQLFIH SGLLSKPEIE QILATPQAKY | ID |
| subsp. torquens | SVYELRVAGL DRRLTNSELF RVLYFFIGHR GFKSNRKAEL NPENEADKKQ | NO: |
| KCTC 3535 | MGQLLNSIEE IRKAIAEKGY RTVGELYLKD PKYNDHKRNK GYIDGYLSTP | 82) |
| WP_010014406.1 | NRQMLVDEIK QILDKQRELG NEKLTDEFYA TYLLGDENRA GIFQAQRDFD | |
| | EGPGAGPYAG DQIKKMVGKD IFEPTEDRAA KATYTFQYFN LLQKMTSLNY | |
| | QNTTGDTWHT LNGLDRQAII DAVFAKAEKP TKTYKPTDFG ELRKLLKLPD | |
| | DARFNLVNYG SLQTQKEIET VEKKTRFVDF KAYHDLVKVL PEEMWQSRQL | |
| | LDHIGTALTL YSSDKRRRRY FAEELNLPAE LIEKLLPLNF SKFGHLSIKS | |
| | MQNIIPYLEM GQVYSEATTN TGYDFRKKQI SKDTIREEIT NPVVRRAVTK | |
| | TIKIVEQIIR RYGKPDGINI ELARELGRNF KERGDIQKRQ DKNRQTNDKI | |
| | AAELTELGIP VNGQNIIRYK LHKEQNGVDP YTGDQIPFER AFSEGYEVDH | |
| | IIPYSISWDD SYTNKVLTSA KCNREKGNRI PMVYLANNEQ RLNALTNIAD | |
| | NIIRNSRKRQ KLLKQKLSDE ELKDWKQRNI NDTRFITRVL YNYFRQAIEF | |
| | NPELEKKQRV LPLNGEVTSK IRSRWGFLKV REDGDLHHAI DATVIAAITP | |
| | KFIQQVTKYS QHQEVKNNQA LWHDAEIKDA EYAAEAQRMD ADLFNKIFNG | |
| | FPLPWPEFLD ELLARISDNP VEMMKSRSWN TYTPIEIAKL KPVFVVRLAN | |
| | HKISGPAHLD TIRSAKLFDE KGIVLSRVSI TKLKINKKGQ VATGDGIYDP | |
| | ENSNNGDKVV YSAIRQALEA HNGSGELAFP DGYLEYVDHG TKKLVRKVRV | |
| | AKKVSLPVRL KNKAAADNGS MVRIDVFNTG KKFVFVPIYI KDTVEQVLPN | |
| | KAIARGKSLW YQITESDQFC FSLYPGDMVH IESKTGIKPK YSNKENNTSV | |
| | VPIKNFYGYF DGADIATASI LVRAHDSSYT ARSIGIAGLL KFEKYQVDYF | |
| | GRYHKVHEKK RQLFVKRDE | |
| Ignavibacterium | MEFKKVLGLD IGTNSIGCAL LSLPKSIQDY GKGGRLEWLT SRVIPLDADY | (SEQ |
| album JCM | MKAFIDGKNG LPQVITPAGK RRQKRGSRRL KHRYKLRRSR LIRVFKTLNW | ID |
| 16511 | LPEDFPLDNP KRIKETISTE GKFSFRISDY VPISDESYRE FYREFGYPEN | NO: |
| WP_014561873.1 | EIEQVIEEIN FRRKTKGKNK NPMIKLLPED WVVYYLRKKA LIKPTTKEEL | 83) |
| | IRIIYLFNQR RGFKSSRKDL TETAILDYDE FAKRLAEKEK YSAENYETKF | |
| | VSITKVKEVV ELKTDGRKGK KRFKVILEDS RIEPYEIERK EKPDWEGKEY | |
| | TFLVTQKLEK GKFKQNKPDL PKEEDWALCT TALDNRMGSK HPGEFFFDEL | |
| | LKAFKEKRGY KIRQYPVNRW RYKKELEFIW TKQCQLNPEL NNLNINKEIL | |
| | RKLATVLYPS QSKFFGPKIK EFENSDVLHI ISEDIIYYQR DLKSQKSLIS | |
| | ECRYEKRKGI DGEIYGLKCI PKSSPLYQEF RIWQDIHNIK VIRKESEVNG | |
| | KKKINIDETQ LYINENIKEK LFELFNSKDS LSEKDILELI SLNIINSGIK | |
| | ISKKEEETTH RINLFANRKE LKGNETKSRY RKVFKKLGFD GEYILNHPSK | |
| | LNRLWHSDYS NDYADKEKTE KSILSSLGWK NRNGKWEKSK NYDVFNLPLE | |
| | VAKAIANLPP LKKEYGSYSA LAIRKMLVVM RDGKYWQHPD QIAKDQENTS | |
| | LMLFDKNLIQ LTNNQRKVLN KYLLTLAEVQ KRSTLIKQKL NEIEHNPYKL | |
| | ELVSDQDLEK QVLKSFLEKK NESDYLKGLK TYQAGYLIYG KHSEKDVPIV | |
| | NSPDELGEYI RKKLPNNSLR NPIVEQVIRE TIFIVRDVWK SFGIIDEIHI | |
| | ELGRELKNNS EERKKTSESQ EKNFQEKERA RKLLKELLNS SNFEHYDENG | |
| | NKIFSSFTVN PNPDSPLDIE KFRIWKNQSG LTDEELNKKL KDEKIPTEIE | |
| | VKKYILWLTQ KCRSPYTGKI IPLSKLFDSN VYEIEHIIPR SKMKNDSTNN | |
| | LVICELGVNK AKGDRLAANF ISESNGKCKF GEVEYTLLKY GDYLQYCKDT | |
| | FKYQKAKYKN LLATEPPEDF IERQINDTRY IGRKLAELLT PVVKDSKNII | |
| | FTIGSITSEL KITWGLNGVW KDILRPRFKR LESIINKKLI FQDEDDPNKY | |
| | HFDLSINPQL DKEGLKRLDH RHHALDATII AATTREHVRY LNSLNAADND | |
| | EEKREYFLSL CNHKIRDFKL PWENFTSEVK SKLLSCVVSY KESKPILSDP | |
| | FNKYLKWEYK NGKWQKVFAI QIKNDRWKAV RRSMFKEPIG TVWIKKIKEV | |
| | SLKEAIKIQA IWEEVKNDPV RKKKEKYIYD DYAQKVIAKI VQELGLSSSM | |
| | RKQDDEKLNK FINEAKVSAG VNKNLNTTNK TIYNLEGRFY EKIKVAEYVL | |
| | YKAKRMPLNK KEYIEKLSLQ KMFNDLPNFI LEKSILDNYP EILKELESDN | |
| | KYIIEPHKKN NPVNRLLLEH ILEYHNNPKE AFSTEGLEKL NKKAINKIGK | |
| | PIKYITRLDG DINEEEIFRG AVFETDKGSN VYFVMYENNQ TKDREFLKPN | |
| | PSISVLKAIE HKNKIDFFAP NRLGFSRIIL SPGDLVYVPT NDQYVLIKDN | |
| | SSNETIINWD DNEFISNRIY QVKKFTGNSC YFLKNDIASL ILSYSASNGV | |
| | GEFGSQNISE YSVDDPPIRI KDVCIKIRVD RLGNVRPL | |
| uncultured delta | MSSKAIDSLE QLDLFKPQEY TLGLDLGIKS IGWAILSGER IANAGVYLFE | (SEQ |
| proteobacterium | TAEELNSTGN KLISKAAERG RKRRIRRMLD RKARRGRHIR YLLEREGLPT | ID |
| HF0070_07E19 | DELEEVVVHQ SNRTLWDVRA EAVERKLTKQ ELAAVLFHLV RHRGYFPNTK | NO: |
| ADI19058.1 | KLPPDDESDS ADEEQGKINR ATSRLREELK ASDCKTIGQF LAQNRDRQRN | 84) |
| | REGDYSNLMA RKLVFEEALQ ILAFQRKQGH ELSKDFEKTY LDVLMGQRSG | |
| | RSPKLGNCSL IPSELRAPSS APSTEWFKFL QNLGNLQISN AYREEWSIDA | |
| | PRRAQIIDAC SQRSTSSYWQ IRRDFQIPDE YRFNLVNYER RDPDVDLQEY | |
| | LQQQERKTLA NFRNWKQLEK IIGTGHPIQT LDEAARLITL IKDDEKLSDQ | |
| | LADLLPEASD KAITQLCELD FTTAAKISLE AMYRILPHMN QGMGFFDACQ | |
| | QESLPEIGVP PAGDRVPPFD EMYNPVVNRV LSQSRKLINA VIDEYGMPAK | |
| | IRVELARDLG KGRELRERIK LDQLDKSKQN DQRAEDFRAE FQQAPRGDQS | |
| | LRYRLWKEQN CTCPYSGRMI PVNSVLSEDT QIDHILPISQ SFDNSLSNKV | |
| | LCFTEENAQK SNRTPFEYLD AADFQRLEAI SGNWPEAKRN KLLHKSFGKV | |
| | AEEWKSRALN DTRYLTSALA DHLRHHLPDS KIQTVNGRIT GYLRKQWGLE | |
| | KDRDKHTHHA VDAIVVACTT PAIVQQVTLY HQDIRRYKKL GEKRPTPWPE | |
| | TFRQDVLDVE EEIFITRQPK KVSGGIQTKD TLRKHRSKPD RQRVALTKVK | |
| | LADLERLVEK DASNRNLYEH LKQCLEESGD QPTKAFKAPF YMPSGPEAKQ | |
| | RPILSKVTLL REKPEPPKQL TELSGGRRYD SMAQGRLDIY RYKPGGKRKD | |
| | EYRVVLQRMI DLMRGEENVH VFQKGVPYDQ GPEIEQNYTF LFSLYFDDLV | |
| | EFQRSADSEV IRGYYRTFNI ANGQLKISTY LEGRQDFDFF GANRLAHFAK | |
| | VQVNLLGKVI K | |
| Ruminococcus | MGNYYLGLDV GIGSIGWAVI NIEKKRIEDF NVRIFKSGEI QEKNRNSRAS | (SEQ |
| albus 8 | QQCRRSRGLR RLYRRKSHRK LRLKNYLSII GLTTSEKIDY YYETADNNVI | ID |
| WP_002846926.1 | QLRNKGLSEK LTPEEIAACL IHICNNRGYK DFYEVNVEDI EDPDERNEYK | NO: |
| | EEHDSIVLIS NLMNEGGYCT PAEMICNCRE FDEPNSVYRK FHNSAASKNH | 85) |
| | YLITRHMLVK EVDLILENQS KYYGILDDKT IAKIKDIIFA QRDFEIGPGK | |
| | NERFRRFTGY LDSIGKCQFF KDQERGSRFT VIADIYAFVN VLSQYTYTNN | |
| | RGESVFDTSF ANDLINSALK NGSMDKRELK AIAKSYHIDI SDKNSDTSLT | |
| | KCFKYIKVVK PLFEKYGYDW DKLIENYTDT DNNVLNRIGI VLSQAQTPKR | |
| | RREKLKALNI GLDDGLINEL TKLKLSGTAN VSYKYMQGSI EAFCEGDLYG | |
| | KYQAKFNKEI PDIDENAKPQ KLPPFKNEDD CEFFKNPVVF RSINETRKLI | |
| | NAIIDKYGYP AAVNIETADE LNKTFEDRAI DTKRNNDNQK ENDRIVKEII | |
| | ECIKCDEVHA RHLIEKYKLW EAQEGKCLYS GETITKEDML RDKDKLFEVD | |
| | HIVPYSLILD NTINNKALVY AEENQKKGQR TPLMYMNEAQ AADYRVRVNT | |
| | MFKSKKCSKK KYQYLMLPDL NDQELLGGWR SRNLNDTRYI CKYLVNYLRK | |
| | NLRFDRSYES SDEDDLKIRD HYRVFPVKSR FTSMFRRWWL NEKTWGRYDK | |
| | AELKKLTYLD HAADAIIIAN CRPEYVVLAG EKLKLNKMYH QAGKRITPEY | |
| | EQSKKACIDN LYKLFRMDRR TAEKLLSGHG RLTPIIPNLS EEVDKRLWDK | |
| | NIYEQFWKDD KDKKSCEELY RENVASLYKG DPKFASSLSM PVISLKPDHK | |
| | YRGTITGEEA IRVKEIDGKL IKLKRKSISE ITAESINSIY TDDKILIDSL | |
| | KTIFEQADYK DVGDYLKKTN QHFFTTSSGK RVNKVTVIEK VPSRWLRKEI | |
| | DDNNFSLLND SSYYCIELYK DSKGDNNLQG IAMSDIVHDR KTKKLYLKPD | |
| | FNYPDDYYTH VMYIFPGDYL RIKSTSKKSG EQLKFEGYFI SVKNVNENSF | |
| | RFISDNKPCA KDKRVSITKK DIVIKLAVDL MGKVQGENNG KGISCGEPLS | |
| | LLKEKN | |
| Lactobacillus | MTKKEQPYNI GLDIGTSSVG WAVTNDNYDL LNIKKKNLWG VRLFEEAQTA | (SEQ |
| farciminis | KETRLNRSTR RRYRRRKNRI NWLNEIFSEE LAKTDPSFLI RLQNSWVSKK | ID |
| KCTC 3681 | DPDRKRDKYN LFIDGPYTDK EYYREFPTIF HLRKELILNK DKADIRLIYL | NO: |
| WP_010018949.1 | ALHNILKYRG NFTYEHQKFN ISNLNNNLSK ELIELNQQLI KYDISFPDDC | 86) |
| | DWNHISDILI GRGNATQKSS NILKDFTLDK ETKKLLKEVI NLILGNVAHL | |
| | NTIFKTSLTK DEEKLNFSGK DIESKLDDLD SILDDDQFTV LDAANRIYST | |
| | ITLNEILNGE SYFSMAKVNQ YENHAIDLCK LRDMWHTTKN EEAVEQSRQA | |
| | YDDYINKPKY GTKELYTSLK KFLKVALPTN LAKEAEEKIS KGTYLVKPRN | |
| | SENGVVPYQL NKIEMEKIID NQSQYYPFLK ENKEKLLSIL SFRIPYYVGP | |
| | LQSAEKNPFA WMERKSNGHA RPWNFDEIVD REKSSNKFIR RMTVTDSYLV | |
| | GEPVLPKNSL IYQRYEVLNE LNNIRITENL KTNPIGSRLT VETKQRIYNE | |
| | LFKKYKKVTV KKLTKWLIAQ GYYKNPILIG LSQKDEFNST LTTYLDMKKI | |
| | FGSSFMEDNK NYDQIEELIE WLTIFEDKQI LNEKLHSSKY SYTPDQIKKI | |
| | SNMRYKGWGR LSKKILMDIT TETNTPQLLQ LSNYSILDLM WATNNNFISI | |
| | MSNDKYDFKN YIENHNLNKN EDQNISDLVN DIHVSPALKR GITQSIKIVQ | |
| | EIVKFMGHAP KHIFIEVTRE TKKSEITTSR EKRIKRLQSK LLNKANDFKP | |
| | QLREYLVPNK KIQEELKKHK NDLSSERIML YFLQNGKSLY SEESLNINKL | |
| | SDYQVDHILP RTYIPDDSLE NKALVLAKEN QRKADDLLLN SNVIDRNLER | |
| | WTYMLNNNMI GLKKFKNLTR RVITDKDKLG FIHRQLVQTS QMVKGVANIL | |
| | DNMYKNQGTT CIQARANLST AFRKALSGQD DTYHFKHPEL VKNRNVNDFH | |
| | HAQDAYLASF LGTYRLRRFP TNEMLLMNGE YNKFYGQVKE LYSKKKKLPD | |
| | SRKNGFIISP LVNGTTQYDR NTGEIIWNVG FRDKILKIFN YHQCNVTRKT | |
| | EIKTGQFYDQ TIYSPKNPKY KKLIAQKKDM DPNIYGGFSG DNKSSITIVK | |
| | IDNNKIKPVA IPIRLINDLK DKKTLQNWLE ENVKHKKSIQ IIKNNVPIGQ | |
| | IIYSKKVGLL SLNSDREVAN RQQLILPPEH SALLRLLQIP DEDLDQILAF | |
| | YDKNILVEIL QELITKMKKF YPFYKGEREF LIANIENFNQ ATTSEKVNSL | |
| | EELITLLHAN STSAHLIFNN IEKKAFGRKT HGLTLNNTDF IYQSVTGLYE | |
| | TRIHIE | |
| Eubacterium | MMEVFMGRLV LGLDIGITSV GFGIIDLDES EIVDYGVRLF KEGTAAENET | (SEQ |
| dolichum DSM | RRTKRGGRRL KRRRVTRRED MLHLLKQAGI ISTSFHPLNN PYDVRVKGLN | ID |
| 3991 | ERLNGEELAT ALLHLCKHRG SSVETIEDDE AKAKEAGETK KVLSMNDQLL | NO: |
| WP_004800457.1 | KSGKYVCEIQ KERLRTNGHI RGHENNFKTR AYVDEAFQIL SHQDLSNELK | 87) |
| | SAIITIISRK RMYYDGPGGP LSPTPYGRYT YFGQKEPIDL IEKMRGKCSL | |
| | FPNEPRAPKL AYSAELFNLL NDLNNLSIEG EKLTSEQKAM ILKIVHEKGK | |
| | ITPKQLAKEV GVSLEQIRGF RIDTKGSPLL SELTGYKMIR EVLEKSNDEH | |
| | LEDHVFYDEI AEILTKTKDI EGRKKQISEL SSDLNEESVH QLAGLTKFTA | |
| | YHSLSFKALR LINEEMLKTE LNQMQSITLF GLKQNNELSV KGMKNIQADD | |
| | TAILSPVAKR AQRETFKVVN RLREIYGEFD SIVVEMAREK NSEEQRKAIR | |
| | ERQKFFEMRN KQVADIIGDD RKINAKLREK LVLYQEQDGK TAYSLEPIDL | |
| | KLLIDDPNAY EVDHIIPISI SLDDSITNKV LVTHRENQEK GNLTPISAFV | |
| | KGRFTKGSLA QYKAYCLKLK EKNIKTNKGY RKKVEQYLLN ENDIYKYDIQ | |
| | KEFINRNLVD TSYASRVVLN TLTTYFKQNE IPTKVFTVKG SLTNAFRRKI | |
| | NLKKDRDEDY GHHAIDALII ASMPKMRLLS TIFSRYKIED IYDESTGEVF | |
| | SSGDDSMYYD DRYFAFIASL KAIKVRKFSH KIDTKPNRSV ADETIYSTRV | |
| | IDGKEKVVKK YKDIYDPKFT ALAEDILNNA YQEKYLMALH DPQTFDQIVK | |
| | VVNYYFEEMS KSEKYFTKDK KGRIKISGMN PLSLYRDEHG MLKKYSKKGD | |
| | GPAITQMKYF DGVLGNHIDI SAHYQVRDKK VVLQQISPYR TDFYYSKENG | |
| | YKFVTIRYKD VRWSEKKKKY VIDQQDYAMK KAEKKIDDTY EFQFSMHRDE | |
| | LIGITKAEGE ALIYPDETWH NFNFFFHAGE TPEILKFTAT NNDKSNKIEV | |
| | KPIHCYCKMR LMPTISKKIV RIDKYATDVV GNLYKVKKNT LKFEFD | |
| Nitratifractor | MKKILGVDLG ITSFGYAILQ ETGKDLYRCL DNSVVMRNNP YDEKSGESSQ | (SEQ |
| salsuginis | SIRSTQKSMR RLIEKRKKRI RCVAQTMERY GILDYSETMK INDPKNNPIK | ID |
| DSM 16511 | NRWQLRAVDA WKRPLSPQEL FAIFAHMAKH RGYKSIATED LIYELELELG | NO: |
| ADV46720.1 | LNDPEKESEK KADERRQVYN ALRHLEELRK KYGGETIAQT IHRAVEAGDL | 88) |
| | RSYRNHDDYE KMIRREDIEE EIEKVLLRQA ELGALGLPEE QVSELIDELK | |
| | ACITDQEMPT IDESLFGKCT FYKDELAAPA YSYLYDLYRL YKKLADLNID | |
| | GYEVTQEDRE KVIEWVEKKI AQGKNLKKIT HKDLRKILGL APEQKIFGVE | |
| | DERIVKGKKE PRTFVPFFFL ADIAKFKELF ASIQKHPDAL QIFRELAEIL | |
| | QRSKTPQEAL DRLRALMAGK GIDTDDRELL ELFKNKRSGT RELSHRYILE | |
| | ALPLFLEGYD EKEVQRILGF DDREDYSRYP KSLRHLHLRE GNLFEKEENP | |
| | INNHAVKSLA SWALGLIADL SWRYGPFDEI ILETTRDALP EKIRKEIDKA | |
| | MREREKALDK IIGKYKKEFP SIDKRLARKI QLWERQKGLD LYSGKVINLS | |
| | QLLDGSADIE HIVPQSLGGL STDYNTIVTL KSVNAAKGNR LPGDWLAGNP | |
| | DYRERIGMLS EKGLIDWKKR KNLLAQSLDE IYTENTHSKG IRATSYLEAL | |
| | VAQVLKRYYP FPDPELRKNG IGVRMIPGKV TSKTRSLLGI KSKSRETNFH | |
| | HAEDALILST LTRGWQNRLH RMLRDNYGKS EAELKELWKK YMPHIEGLTL | |
| | ADYIDEAFRR FMSKGEESLF YRDMFDTIRS ISYWVDKKPL SASSHKETVY | |
| | SSRHEVPTLR KNILEAFDSL NVIKDRHKLT TEEFMKRYDK EIRQKLWLHR | |
| | IGNTNDESYR AVEERATQIA QILTRYQLMD AQNDKEIDEK FQQALKELIT | |
| | SPIEVTGKLL RKMRFVYDKL NAMQIDRGLV ETDKNMLGIH ISKGPNEKLI | |
| | FRRMDVNNAH ELQKERSGIL CYLNEMLFIF NKKGLIHYGC LRSYLEKGQG | |
| | SKYIALFNPR FPANPKAQPS KFTSDSKIKQ VGIGSATGII KAHLDLDGHV | |
| | RSYEVFGTLP EGSIEWFKEE SGYGRVEDDP HH | |
| Rhodospirillum | MRPIEPWILG LDIGTDSLGW AVFSCEEKGP PTAKELLGGG VRLFDSGRDA | (SEQ |
| rubrum | KDHTSRQAER GAFRRARRQT RTWPWRRDRL IALFQAAGLT PPAAETRQIA | ID |
| ATCC 11170 | LALRREAVSR PLAPDALWAA LLHLAHHRGF RSNRIDKRER AAAKALAKAK | NO |
| WP_011388212.1 | PAKATAKATA PAKEADDEAG FWEGAEAALR QRMAASGAPT VGALLADDLD | 89) |
| | RGQPVRMRYN QSDRDGVVAP TRALIAEELA EIVARQSSAY PGLDWPAVTR | |
| | LVLDQRPLRS KGAGPCAFLP GEDRALRALP TVQDFIIRQT LANLRLPSTS | |
| | ADEPRPLTDE EHAKALALLS TARFVEWPAL RRALGLKRGV KFTAETERNG | |
| | AKQAARGTAG NLTEAILAPL IPGWSGWDLD RKDRVFSDLW AARQDRSALL | |
| | ALIGDPRGPT RVTEDETAEA VADAIQIVLP TGRASLSAKA ARAIAQAMAP | |
| | GIGYDEAVTL ALGLHHSHRP RQERLARLPY YAAALPDVGL DGDPVGPPPA | |
| | EDDGAAAEAY YGRIGNISVH IALNETRKIV NALLHRHGPI LRLVMVETTR | |
| | ELKAGADERK RMIAEQAERE RENAEIDVEL RKSDRWMANA RERRQRVRLA | |
| | RRQNNLCPYT STPIGHADLL GDAYDIDHVI PLARGGRDSL DNMVLCQSDA | |
| | NKTKGDKTPW EAFHDKPGWI AQRDDFLARL DPQTAKALAW RFADDAGERV | |
| | ARKSAEDEDQ GFLPRQLTDT GYIARVALRY LSLVTNEPNA VVATNGRLTG | |
| | LLRLAWDITP GPAPRDLLPT PRDALRDDTA ARRFLDGLTP PPLAKAVEGA | |
| | VQARLAALGR SRVADAGLAD ALGLTLASLG GGGKNRADHR HHFIDAAMIA | |
| | VTTRGLINQI NQASGAGRIL DLRKWPRTNF EPPYPTFRAE VMKQWDHIHP | |
| | SIRPAHRDGG SLHAATVFGV RNRPDARVLV QRKPVEKLFL DANAKPLPAD | |
| | KIAEIIDGFA SPRMAKRFKA LLARYQAAHP EVPPALAALA VARDPAFGPR | |
| | GMTANTVIAG RSDGDGEDAG LITPFRANPK AAVRTMGNAV YEVWEIQVKG | |
| | RPRWTHRVLT RFDRTQPAPP PPPENARLVM RLRRGDLVYW PLESGDRLFL | |
| | VKKMAVDGRL ALWPARLATG KATALYAQLS CPNINLNGDQ GYCVQSAEGI | |
| | RKEKIRTTSC TALGRLRLSK KAT | |
| Finegoldia | MKSEKKYYIG LDVGTNSVGW AVTDEFYNIL RAKGKDLWGV RLFEKADTAA | (SEQ |
| magna ATCC | NTRIFRSGRR RNDRKGMRLQ ILREIFEDEI KKVDKDFYDR LDESKFWAED | ID |
| 29328 | KKVSGKYSLF NDKNFSDKQY FEKFPTIFHL RKYLMEEHGK VDIRYYFLAI | NO: |
| WP_012290141.1 | NQMMKRRGHF LIDGQISHVT DDKPLKEQLI LLINDLLKIE LEEELMDSIF | 90) |
| | EILADVNEKR TDKKNNLKEL IKGQDFNKQE GNILNSIFES IVTGKAKIKN | |
| | IISDEDILEK IKEDNKEDFV LTGDSYEENL QYFEEVLQEN ITLFNTLKST | |
| | YDFLILQSIL KGKSTLSDAQ VERYDEHKKD LEILKKVIKK YDEDGKLFKQ | |
| | VFKEDNGNGY VSYIGYYLNK NKKITAKKKI SNIEFTKYVK GILEKQCDCE | |
| | DEDVKYLLGK IEQENFLLKQ ISSINSVIPH QIHLFELDKI LENLAKNYPS | |
| | FNNKKEEFTK IEKIRKTFTF RIPYYVGPLN DYHKNNGGNA WIFRNKGEKI | |
| | RPWNFEKIVD LHKSEEEFIK RMLNQCTYLP EETVLPKSSI LYSEYMVLNE | |
| | LNNLRINGKP LDTDVKLKLI EELFKKKTKV TLKSIRDYMV RNNFADKEDF | |
| | DNSEKNLEIA SNMKSYIDFN NILEDKFDVE MVEDLIEKIT IHTGNKKLLK | |
| | KYIEETYPDL SSSQIQKIIN LKYKDWGRLS RKLLDGIKGT KKETEKTDTV | |
| | INFLRNSSDN LMQIIGSQNY SFNEYIDKLR KKYIPQEISY EVVENLYVSP | |
| | SVKKMIWQVI RVTEEITKVM GYDPDKIFIE MAKSEEEKKT TISRKNKLLD | |
| | LYKAIKKDER DSQYEKLLTG LNKLDDSDLR SRKLYLYYTQ MGRDMYTGEK | |
| | IDLDKLFDST HYDKDHIIPQ SMKKDDSIIN NLVLVNKNAN QTTKGNIYPV | |
| | PSSIRNNPKI YNYWKYLMEK EFISKEKYNR LIRNTPLTNE ELGGFINRQL | |
| | VETRQSTKAI KELFEKFYQK SKIIPVKASL ASDLRKDMNT LKSREVNDLH | |
| | HAHDAFLNIV AGDVWNREFT SNPINYVKEN REGDKVKYSL SKDFTRPRKS | |
| | KGKVIWTPEK GRKLIVDTLN KPSVLISNES HVKKGELFNA TIAGKKDYKK | |
| | GKIYLPLKKD DRLQDVSKYG GYKAINGAFF FLVEHTKSKK RIRSIELFPL | |
| | HLLSKFYEDK NTVLDYAINV LQLQDPKIII DKINYRTEII IDNFSYLIST | |
| | KSNDGSITVK PNEQMYWRVD EISNLKKIEN KYKKDAILTE EDRKIMESYI | |
| | DKIYQQFKAG KYKNRRTTDT IIEKYEIIDL DTLDNKQLYQ LLVAFISLSY | |
| | KTSNNAVDFT VIGLGTECGK PRITNLPDNT YLVYKSITGI YEKRIRIK | |
| Eubacterium | MNYTEKEKLF MKYILALDIG IASVGWAILD KESETVIEAG SNIFPEASAA | (SEQ |
| rectale ATCC | DNQLRRDMRG AKRNNRRLKT RINDFIKLWE NNNLSIPQFK STEIVGLKVR | ID |
| 33656 | AITEEITLDE LYLILYSYLK HRGISYLEDA LDDTVSGSSA YANGLKLNAK | NO: |
| WP_012742555.1 | ELETHYPCEI QQERLNTIGK YRGQSQIINE NGEVLDLSNV FTIGAYRKEI | 91) |
| | QRVFEIQKKY HPELTDEFCD GYMLIFNRKR KYYEGPGNEK SRTDYGRFTT | |
| | KLDANGNYIT EDNIFEKLIG KCSVYPDELR AAAASYTAQE YNVLNDLNNL | |
| | TINGRKLEEN EKHEIVERIK SSNTINMRKI ISDCMGENID DFAGARIDKS | |
| | GKEIFHKFEV YNKMRKALLE IGIDISNYSR EELDEIGYIM TINTDKEAMM | |
| | EAFQKSWIDL SDDVKQCLIN MRKTNGALFN KWQSFSLKIM NELIPEMYAQ | |
| | PKEQMTLLTE MGVTKGTQEE FAGLKYIPVD VVSEDIFNPV VRRSVRISFK | |
| | ILNAVLKKYK ALDTIVIEMP RDRNSEEQKK RINDSQKLNE KEMEYIEKKL | |
| | AVTYGIKLSP SDFSSQKQLS LKLKLWNEQD GICLYSGKTI DPNDIINNPQ | |
| | LFEIDHIIPR SISFDDARSN KVLVYRSENQ KKGNQTPYYY LTHSHSEWSF | |
| | EQYKATVMNL SKKKEYAISR KKIQNLLYSE DITKMDVLKG FINRNINDTS | |
| | YASRLVLNTI QNFFMANEAD TKVKVIKGSY THQMRCNLKL DKNRDESYSH | |
| | HAVDAMLIGY SELGYEAYHK LQGEFIDFET GEILRKDMWD ENMSDEVYAD | |
| | YLYGKKWANI RNEVVKAEKN VKYWHYVMRK SNRGLCNQTI RGTREYDGKQ | |
| | YKINKLDIRT KEGIKVFAKL AFSKKDSDRE RLLVYLNDRR TFDDLCKIYE | |
| | DYSDAANPFV QYEKETGDII RKYSKKHNGP RIDKLKYKDG EVGACIDISH | |
| | KYGFEKGSKK VILESLVPYR MDVYYKEENH SYYLVGVKQS DIKFEKGRNV | |
| | IDEEAYARIL VNEKMIQPGQ SRADLENLGF KFKLSFYKND IIEYEKDGKI | |
| | YTERLVSRTM PKQRNYIETK PIDKAKFEKQ NLVGLGKTKF IKKYRYDILG | |
| | NKYSCSEEKF TSFC | |
| Corynebacterium | MKYHVGIDVG TFSVGLAAIE VDDAGMPIKT LSLVSHIHDS GLDPDKIKSA | (SEQ |
| diphtheriae | VTRLASSGIA RRTRRLYRRK RRRLQQLDKF IQRQGWPVIE LEDYSDPLYP | ID |
| C7 (beta) | WKVRAELAAS YIADEKERGE KLSVALRHIA RHRGWRNPYA KVSSLYLPDE | NO: |
| AEX66236.1 | PSDAFKAIRE EIKRASGQPV PETATVGQMV TLCELGTLKL RGEGGVLSAR | |
| WP_014318431.1 | LQQSDHAREI QEICRMQEIG QELYRKIIDV VFAAESPKGS ASSRVGKDPL | 92) |
| | QPGKNRALKA SDAFQRYRIA ALIGNLRVRV DGEKRILSVE EKNLVFDHLV | |
| | NLAPKKEPEW VTIAEILGID RGQLIGTATM TDDGERAGAR PPTHDTNRSI | |
| | VNSRIAPLVD WWKTASALEQ HAMVKALSNA EVDDFDSPEG AKVQAFFADL | |
| | DDDVHAKLDS LHLPVGRAAY SEDTLVRLTR RMLADGVDLY TARLQEFGIE | |
| | PSWTPPAPRI GEPVGNPAVD RVLKTVSRWL ESATKTWGAP I ERVIIEHVRE | |
| | GFVTEKRARE MDGDMRRRAA RNAKLFQEMQ EKLNVQGKPS RADLWRYQSV | |
| | QRQNCQCAYC GSPITFSNSE MDHIVPRAGQ GSTNTRENLV AVCHRCNQSK | |
| | GNTPFAIWAK NTSIEGVSVK EAVERTRHWV TDTGMRSTDF KKFTKAVVER | |
| | FQRATMDEEI DARSMESVAW MANELRSRVA QHFASHGTTV RVYRGSLTAE | |
| | ARRASGISGK LEFLDGVGKS RLDRRHHAID AAVIAFTSDY VAETLAVRSN | |
| | LKQSQAHRQE APQWREFTGK DAEHRAAWRV WCQKMEKLSA LLTEDLRDDR | |
| | VVVMSNVRLR LGNGSAHEET IGKLSKVKLG SQLSVSDIDK ASSEALWCAL | |
| | TREPDFDPKD GLPANPERHI RVNGTHVYAG DNIGLFPVSA GSIALRGGYA | |
| | ELGSSFHHAR VYKITSGKKP AFAMLRVYTI DLLPYRNQDL FSVELKPQTM | |
| | SMRQAEKKLR DALATGNAEY LGWLVVDDEL VVDTSKIATD QVKAVEAELG | |
| | TIRRWRVDGF FGDTRLRLRP LQMSKEGIKK ESAPELSKII DRPGWLPAVN | |
| | KLFSEGNVTV VRRDSLGRVR LESTAHLPVT WKVQ | |
| Roseburia | MNAEHGKEGL LIMEENFQYR IGLDIGITSV GWAVLQNNSQ DEPVRITDLG | (SEQ |
| inulinivorans | VRIFDVAENP KNGDALAAPR RDARTTRRRL RRRRHRLERI KFLLQENGLI | ID |
| DSM 16841 | EMDSFMERYY KGNLPDVYQL RYEGLDRKLK DEELAQVLIH IAKHRGFRST | NO: |
| WP_007889305.1 | RKAETKEKEG GAVLKATTEN QKIMQEKGYR TVGEMLYLDE AFHTECLWNE | 93) |
| | KGYVLTPRNR PDDYKHTILR SMLVEEVHAI FAAQRAHGNQ KATEGLEEAY | |
| | VEIMTSQRSF DMGPGLQPDG KPSPYAMEGF GDRVGKCTFE KDEYRAPKAT | |
| | YTAELFVALQ KINHTKLIDE FGTGRFFSEE ERKTIIGLLL SSKELKYGTI | |
| | RKKLNIDPSL KFNSLNYSAK KEGETEEERV LDTEKAKFAS MFWTYEYSKC | |
| | LKDRTEEMPV GEKADLFDRI GEILTAYKND DSRSSRLKEL GLSGEEIDGL | |
| | LDLSPAKYQR VSLKAMRKMQ PYLEDGLIYD KACEAAGYDF RALNDGNKKH | |
| | LLKGEEINAI VNDITNPVVK RSVSQTIKVI NAIIQKYGSP QAVNIELARE | |
| | MSKNFQDRTN LEKEMKKRQQ ENERAKQQII ELGKQNPTGQ DILKYRLWND | |
| | QGGYCLYSGK KIPLEELFDG GYDIDHILPY SITFDDSYRN KVLVTAQENR | |
| | QKGNRTPYEY FGADEKRWED YEASVRLLVR DYKKQQKLLK KNFTEEERKE | |
| | FKERNLNDTK YITRVVYNMI RQNLELEPFN HPEKKKQVWA VNGAVTSYLR | |
| | KRWGLMQKDR STDRHHAMDA VVIACCTDGM IHKISRYMQG RELAYSRNFK | |
| | FPDEETGEIL NRDNFTREQW DEKFGVKVPL PWNSFRDELD IRLLNEDPKN | |
| | FLLTHADVQR ELDYPGWMYG EEESPIEEGR YINYIRPLFV SRMPNHKVTG | |
| | SAHDATIRSA RDYETRGVVI TKVPLTDLKL NKDNEIEGYY DKDSDRLLYQ | |
| | ALVRQLLLHG NDGKKAFAED FHKPKADGTE GPVVRKVKIE KKQTSGVMVR | |
| | GGTGIAANGE MVRIDVFREN GKYYFVPVYT ADVVRKVLPN RAATHTKPYS | |
| | EWRVMDDANF VFSLYSRDLI HVKSKKDIKT NLVNGGLLLQ KEIFAYYTGA | |
| | DIATASIAGF ANDSNFKFRG LGIQSLEIFE KCQVDILGNI SVVRHENRQE | |
| | FH | |
| Alicycliphilus | MRSLRYRLAL DLGSTSLGWA LFRLDACNRP TAVIKAGVRI FSDGRNPKDG | (SEQ |
| denitrificans | SSLAVTRRAA RAMRRRRDRL LKRKTRMQAK LVEHGFFPAD AGKRKALEQL | ID |
| K601 | NPYALRAKGL QEALLPGEFA RALFHINQRR GFKSNRKTDK KDNDSGVLKK | NO: |
| WP_013517127.1 | AIGQLRQQMA EQGSRTVGEY LWTRLQQGQG VRARYREKPY TTEEGKKRID | 94) |
| | KSYDLYIDRA MIEQEFDALW AAQAAFNPTL FHEAARADLK DTLLHQRPLR | |
| | PVKPGRCTLL PEEERAPLAL PSTQRFRIHQ EVNHLRLLDE NLREVALTLA | |
| | QRDAVVTALE TKAKLSFEQI RKLLKLSGSV QFNLEDAKRT ELKGNATSAA | |
| | LARKELFGAA WSGFDEALQD EIVWQLVTEE GEGALIAWLQ THTGVDEARA | |
| | QAIVDVSLPE GYGNLSRKAL ARIVPALRAA VITYDKAVQA AGFDHHSQLG | |
| | FEYDASEVED LVHPETGEIR SVFKQLPYYG KALQRHVAFG SGKPEDPDEK | |
| | RYGKIANPTV HIGLNQVRMV VNALIRRYGR PTEVVIELAR DLKQSREQKV | |
| | EAQRRQADNQ RRNARIRRSI AEVLGIGEER VRGSDIQKWI CWEELSFDAA | |
| | DRRCPYSGVQ ISAAMLLSDE VEVEHILPFS KTLDDSLNNR TVAMRQANRI | |
| | KRNRTPWDAR AEFEAQGWSY EDILQRAERM PLRKRYRFAP DGYERWLGDD | |
| | KDFLARALND TRYLSRVAAE YLRLVCPGTR VIPGQLTALL RGKFGLNDVL | |
| | GLDGEKNRND HRHHAVDACV IGVTDQGLMQ RFATASAQAR GDGLTRLVDG | |
| | MPMPWPTYRD HVERAVRHIW VSHRPDHGFE GAMMEETSYG IRKDGSIKQR | |
| | RKADGSAGRE ISNLIRIHEA TQPLRHGVSA DGQPLAYKGY VGGSNYCIEI | |
| | TVNDKGKWEG EVISTFRAYG VVRAGGMGRL RNPHEGQNGR KLIMRLVIGD | |
| | SVRLEVDGAE RTMRIVKISG SNGQIFMAPI HEANVDARNT DKQDAFTYTS | |
| | KYAGSLQKAK TRRVTISPIGEVRDPGFKG | |
| Sphaerochaeta | MSKKVSRRYE EQAQEICQRL GSRPYSIGLD LGVGSIGVAV AAYDPIKKQP | (SEQ |
| globosa str. | SDLVFVSSRI FIPSTGAAER RQKRGQRNSL RHRANRLKFL WKLLAERNLM | ID |
| Buddy | LSYSEQDVPD PARLRFEDAV VRANPYELRL KGLNEQLTLS ELGYALYHIA | NO: |
| WP_013607849.1 | NHRGSSSVRT FLDEEKSSDD KKLEEQQAMT EQLAKEKGIS TFIEVLTAFN | 95) |
| | TNGLIGYRNS ESVKSKGVPV PTRDIISNEI DVLLQTQKQF YQEILSDEYC | |
| | DRIVSAILFE NEKIVPEAGC CPYFPDEKKL PRCHFLNEER RLWEAINNAR | |
| | IKMPMQEGAA KRYQSASFSD EQRHILFHIA RSGTDITPKL VQKEFPALKT | |
| | SIIVLQGKEK AIQKIAGFRF RRLEEKSFWK RLSEEQKDDF FSAWTNTPDD | |
| | KRLSKYLMKH LLLTENEVVD ALKTVSLIGD YGPIGKTATQ LLMKHLEDGL | |
| | TYTEALERGM ETGEFQELSV WEQQSLLPYY GQILTGSTQA LMGKYWHSAF | |
| | KEKRDSEGFF KPNTNSDEEK YGRIANPVVH QTLNELRKLM NELITILGAK | |
| | PQEITVELAR ELKVGAEKRE DIIKQQTKQE KEAVLAYSKY CEPNNLDKRY | |
| | IERFRLLEDQ AFVCPYCLEH ISVADIAAGR ADVDHIFPRD DTADNSYGNK | |
| | VVAHRQCNDI KGKRTPYAAF SNTSAWGPIM HYLDETPGMW RKRRKFETNE | |
| | EEYAKYLQSK GFVSRFESDN SYIAKAAKEY LRCLFNPNNV TAVGSLKGME | |
| | TSILRKAWNL QGIDDLLGSR HWSKDADTSP TMRKNRDDNR HHGLDAIVAL | |
| | YCSRSLVQMI NTMSEQGKRA VEIEAMIPIP GYASEPNLSF EAQRELFRKK | |
| | ILEFMDLHAF VSMKTDNDAN GALLKDTVYS ILGADTQGED LVFVVKKKIK | |
| | DIGVKIGDYE EVASAIRGRI TDKQPKWYPM EMKDKIEQLQ SKNEAALQKY | |
| | KESLVQAAAV LEESNRKLIE SGKKPIQLSE KTISKKALEL VGGYYYLISN | |
| | NKRTKTFVVK EPSNEVKGFA FDTGSNLCLD FYHDAQGKLC GEIIRKIQAM | |
| | NPSYKPAYMK QGYSLYVRLY QGDVCELRAS DLTEAESNLA KTTHVRLPNA | |
| | KPGRTFVIII TFTEMGSGYQ IYFSNLAKSK KGQDTSFTLT TIKNYDVRKV | |
| | QLSSAGLVRY VSPLLVDKIE KDEVALCGE | |
| Fusobacterium | MKKQKFSDYY LGFDIGTNSV GWCVTDLDYN VLRFNKKDMW GSRLFDEAKT | (SEQ |
| nucleatum | AAERRVQRNS RRRLKRRKWR LNLLEEIFSD EIMKIDSNFF RRLKESSLWL | ID |
| subsp. | EDKNSKEKFT LFNDDNYKDY DFYKQYPTIF HLRDELIKNP EKKDIRLIYL | NO: |
| vincentii | ALHSIFKSRG HFLFEGQNLK EIKNFETLYN NLISFLEDNG INKSIDKDNI | 96) |
| ATCC 49256 | EKLEKIICDS GKGLKDKEKE FKGIFNSDKQ LVAIFKLSVG SSVSLNDLFD | |
| WP_005888649.1 | TDEYKKEEVE KEKISFREQI YEDDKPIYYS ILGEKIELLD IAKSFYDFMV | |
| | LNNILSDSNY ISEAKVKLYE EHKKDLKNLK YIIRKYNKEN YDKLFKDKNE | |
| | NNYPAYIGLN KEKDKKEVVE KSRLKIDDLI KVIKGYLPKP ERIEEKDKTI | |
| | FNEILNKIEL KTILPKQRIS DNGTLPYQIH EVELEKILEN QSKYYDFLNY | |
| | EENGVSTKDK LLKTFKFRIP YYVGPLNSYH KDKGGNSWIV RKEEGKILPW | |
| | NFEQKVDIEK SAEEFIKRMT NKCTYLNGED VIPKDSFLYS EYIILNELNK | |
| | VQVNDEFLNE ENKRKIIDEL FKENKKVSEK KFKEYLLVNQ IANRTVELKG | |
| | IKDSFNSNYV SYIKFKDIFG EKLNLDIYKE ISEKSILWKC LYGDDKKIFE | |
| | KKIKNEYGDI LNKDEIKKIN SFKFNTWGRL SEKLLTGIEF INLETGECYS | |
| | SVMEALRRTN YNLMELLSSK FTLQESIDNE NKEMNEVSYR DLIEESYVSP | |
| | SLKRAILQTL KIYEEIKKIT GRVPKKVFIE MARGGDESMK NKKIPARQEQ | |
| | LKKLYDSCGN DIANFSIDIK EMKNSLSSYD NNSLRQKKLY LYYLQFGKCM | |
| | YTGREIDLDR LLQNNDTYDI DHIYPRSKVI KDDSFDNLVL VLKNENAEKS | |
| | NEYPVKKEIQ EKMKSFWRFL KEKNFISDEK YKRLTGKDDF ELRGFMARQL | |
| | VNVRQTTKEV GKILQQIEPE IKIVYSKAEI ASSFREMFDF IKVRELNDTH | |
| | HAKDAYLNIV AGNVYNTKFT EKPYRYLQEI KENYDVKKIY NYDIKNAWDK | |
| | ENSLEIVKKN MEKNTVNITR FIKEEKGELF NLNPIKKGET SNEIISIKPK | |
| | LYDGKDNKLN EKYGYYTSLK AAYFIYVEHE KKNKKVKTFE RITRIDSTLI | |
| | KNEKNLIKYL VSQKKLLNPK IIKKIYKEQT LIIDSYPYTF TGVDSNKKVE | |
| | LKNKKQLYLE KKYEQILKNA LKFVEDNQGE TEENYKFIYL KKRNNNEKNE | |
| | TIDAVKERYN IEFNEMYDKF LEKLSSKDYK NYINNKLYTN FLNSKEKFKK | |
| | LKLWEKSLIL REFLKIFNKN TYGKYEIKDS QTKEKLFSFP EDTGRIRLGQ | |
| | SSLGNNKELL EESVTGLFVK KIKL | |
| Pasteurella | MQTTNLSYIL GLDLGIASVG WAVVEINENE DPIGLIDVGV RIFERAEVPK | (SEQ |
| multocida | TGESLALSRR LARSTRRLIR RRAHRLLLAK RFLKREGILS TIDLEKGLPN | ID |
| subsp. | QAWELRVAGL ERRLSAIEWG AVLLHLIKHR GYLSKRKNES QTNNKELGAL | NO: |
| multocida str. | LSGVAQNHQL LQSDDYRTPA ELALKKFAKE EGHIRNQRGA YTHTFNRLDL | 97) |
| Pm70 | LAELNLLFAQ QHQFGNPHCK EHIQQYMTEL LMWQKPALSG EAILKMLGKC | |
| WP_010907033.1 | THEKNEFKAA KHTYSAERFV WLTKLNNLRI LEDGAERALN EEERQLLINH | |
| | PYEKSKLTYA QVRKLLGLSE QAIFKHLRYS KENAESATFM ELKAWHAIRK | |
| | ALENQGLKDT WQDLAKKPDL LDEIGTAFSL YKTDEDIQQY LTNKVPNSVI | |
| | NALLVSLNFD KFIELSLKSL RKILPLMEQG KRYDQACREI YGHHYGEANQ | |
| | KTSQLLPAIP AQEIRNPVVL RTLSQARKVI NAIIRQYGSP ARVHIETGRE | |
| | LGKSFKERRE IQKQQEDNRT KRESAVQKFK ELFSDFSSEP KSKDILKFRL | |
| | YEQQHGKCLY SGKEINIHRL NEKGYVEIDH ALPFSRTWDD SFNNKVLVLA | |
| | SENQNKGNQT PYEWLQGKIN SERWKNFVAL VLGSQCSAAK KQRLLTQVID | |
| | DNKFIDRNLN DTRYIARFLS NYIQENLLLV GKNKKNVFTP NGQITALLRS | |
| | RWGLIKAREN NNRHHALDAI VVACATPSMQ QKITRFIRFK EVHPYKIENR | |
| | YEMVDQESGE IISPHFPEPW AYFRQEVNIR VFDNHPDTVL KEMLPDRPQA | |
| | NHQFVQPLFV SRAPTRKMSG QGHMETIKSA KRLAEGISVL RIPLTQLKPN | |
| | LLENMVNKER EPALYAGLKA RLAEFNQDPA KAFATPFYKQ GGQQVKAIRV | |
| | EQVQKSGVLV RENNGVADNA SIVRTDVFIK NNKFFLVPIY TWQVAKGILP | |
| | NKAIVAHKNE DEWEEMDEGA KFKFSLFPND LVELKTKKEY FFGYYIGLDR | |
| | ATGNISLKEH DGEISKGKDG VYRVGVKLAL SFEKYQVDEL GKNRQICRPQ | |
| | QRQPVR | |
| Alcanivorax | MRYRVGLDLG TASVGAAVFS MDEQGNPMEL IWHYERLFSE PLVPDMGQLK | (SEQ |
| pacificus W11- | PKKAARRLAR QQRRQIDRRA SRLRRIAIVS RRLGIAPGRN DSGVHGNDVP | ID |
| 5 | TLRAMAVNER IELGQLRAVL LRMGKKRGYG GTFKAVRKVG EAGEVASGAS | NO: |
| WP_008738269.1 | RLEEEMVALA SVQNKDSVTV GEYLAARVEH GLPSKLKVAA NNEYYAPEYA | 98) |
| | LFRQYLGLPA IKGRPDCLPN MYALRHQIEH EFERIWATQS QFHDVMKDHG | |
| | VKEEIRNAIF FQRPLKSPAD KVGRCSLQTN LPRAPRAQIA AQNFRIEKQM | |
| | ADLRWGMGRR AEMLNDHQKA VIRELLNQQK ELSFRKIYKE LERAGCPGPE | |
| | GKGLNMDRAA LGGRDDLSGN TTLAAWRKLG LEDRWQELDE VTQIQVINFL | |
| | ADLGSPEQLD TDDWSCRFMG KNGRPRNFSD EFVAFMNELR MTDGFDRLSK | |
| | MGFEGGRSSY SIKALKALTE WMIAPHWRET PETHRVDEEA AIRECYPESL | |
| | ATPAQGGRQS KLEPPPLTGN EVVDVALRQV RHTINMMIDD LGSVPAQIVV | |
| | EMAREMKGGV TRRNDIEKQN KRFASERKKA AQSIEENGKT PTPARILRYQ | |
| | LWIEQGHQCP YCESNISLEQ ALSGAYTNFE HILPRTLTQI GRKRSELVLA | |
| | HRECNDEKGN RTPYQAFGHD DRRWRIVEQR ANALPKKSSR KTRLLLLKDF | |
| | EGEALTDESI DEFADRQLHE SSWLAKVTTQ WLSSLGSDVY VSRGSLTAEL | |
| | RRRWGLDTVI PQVRFESGMP VVDEEGAEIT PEEFEKFRLQ WEGHRVTREM | |
| | RTDRRPDKRI DHRHHLVDAI VTALTSRSLY QQYAKAWKVA DEKQRHGRVD | |
| | VKVELPMPIL TIRDIALEAV RSVRISHKPD RYPDGRFFEA TAYGIAQRLD | |
| | ERSGEKVDWL VSRKSLTDLA PEKKSIDVDK VRANISRIVG EAIRLHISNI | |
| | FEKRVSKGMT PQQALREPIE FQGNILRKVR CFYSKADDCV RIEHSSRRGH | |
| | HYKMLLNDGF AYMEVPCKEG ILYGVPNLVR PSEAVGIKRA PESGDFIRFY | |
| | KGDTVKNIKT GRVYTIKQIL GDGGGKLILT PVTETKPADL LSAKWGRLKV | |
| | GGRNIHLLRL CAE | |
| Mycoplasma | MYFYKNKENK LNKKVVLGLD LGIASVGWCL TDISQKEDNK FPIILHGVRL | (SEQ |
| mobile 163K | FETVDDSDDK LLNETRRKKR GQRRRNRRLF TRKRDFIKYL IDNNIIELEF | ID |
| AAT27519.1 | DKNPKILVRN FIEKYINPFS KNLELKYKSV TNLPIGFHNL RKAAINEKYK | NO: |
| | LDKSELIVLL YFYLSLRGAF FDNPEDTKSK EMNKNEIEIF DKNESIKNAE | 99) |
| | FPIDKIIEFY KISGKIRSTI NLKFGHQDYL KEIKQVFEKQ NIDFMNYEKF | |
| | AMEEKSFFSR IRNYSEGPGN EKSFSKYGLY ANENGNPELI INEKGQKIYT | |
| | KIFKTLWESK IGKCSYDKKL YRAPKNSFSA KVFDITNKLT DWKHKNEYIS | |
| | ERLKRKILLS RFLNKDSKSA VEKILKEENI KFENLSEIAY NKDDNKINLP | |
| | IINAYHSLTT IFKKHLINFE NYLISNENDL SKLMSFYKQQ SEKLFVPNEK | |
| | GSYEINQNNN VLHIFDAISN ILNKFSTIQD RIRILEGYFE FSNLKKDVKS | |
| | SEIYSEIAKL REFSGTSSLS FGAYYKFIPN LISEGSKNYS TISYEEKALQ | |
| | NQKNNFSHSN LFEKTWVEDL IASPTVKRSL RQTMNLLKEI FKYSEKNNLE | |
| | IEKIVVEVTR SSNNKHERKK IEGINKYRKE KYEELKKVYD LPNENTTLLK | |
| | KLWLLRQQQG YDAYSLRKIE ANDVINKPWN YDIDHIVPRS ISFDDSFSNL | |
| | VIVNKLDNAK KSNDLSAKQF IEKIYGIEKL KEAKENWGNW YLRNANGKAF | |
| | NDKGKFIKLY TIDNLDEFDN SDFINRNLSD TSYITNALVN HLTFSNSKYK | |
| | YSVVSVNGKQ TSNLRNQIAF VGIKNNKETE REWKRPEGFK SINSNDFLIR | |
| | EEGKNDVKDD VLIKDRSFNG HHAEDAYFIT IISQYFRSFK RIERLNVNYR | |
| | KETRELDDLE KNNIKFKEKA SFDNFLLINA LDELNEKLNQ MRFSRMVITK | |
| | KNTQLFNETL YSGKYDKGKN TIKKVEKLNL LDNRTDKIKK IEEFFDEDKL | |
| | KENELTKLHI FNHDKNLYET LKIIWNEVKI EIKNKNLNEK NYFKYFVNKK | |
| | LQEGKISFNE WVPILDNDFK IIRKIRYIKF SSEEKETDEI IFSQSNFLKI | |
| | DQRQNFSFHN TLYWVQIWVY KNQKDQYCFI SIDARNSKFE KDEIKINYEK | |
| | LKTQKEKLQI INEEPILKIN KGDLFENEEK ELFYIVGRDE KPQKLEIKYI | |
| | LGKKIKDQKQ IQKPVKKYFP NWKKVNLTYM GEIFKK | |
| gamma | MTKNYISPIA IDLGAKFTGV ALYQYLEGAD CTQEVAKGLL VDDRGNVTWS | (SEQ |
| proteobacterium | QEGRRGKRHQ VRGYKRRKMA KRLLWLILDS EYGIKREEVT EPLLKFINGL | ID |
| HTCC5015 | LNRRGYTYIS EEVDEESMNV SPLPFSEMMP DYFNSSAPLL EQLAKLLSDK | NO: |
| WP_008284239.1 | NKLVRFRAEG KIPSNKNEFK KLLDTALDGK YKDEKKELSE AWGNILIASE | 100) |
| | NVLKSTVDGH KSRSEYLANI KEDIKSNEEL EKQISSKEID GFYNLVGHLS | |
| | NFQLRLLRKY FNDPNMSGVS YWDEKRLEKY FYQWVQGWHT KGGTDEAEKK | |
| | NIILKTKGAP LLKTLKSLSA DLTIPPYEDQ NNRRPPKCQS VLLSDEKLTM | |
| | HYPKWKEWVG QLVKQNDNAY LNENVTLANA LHRIVERSRS IDPYQLRLLI | |
| | SITDAEKRND LAGYKRLKLS LGSEVDEFLL LVKNIVDETK EAREGLWFET | |
| | ENKLFFKCGK TPPRKEKLKS TLLSAVLGKN LSDDEQSSFI EEFWKSGTPK | |
| | IERRNVRGWC RLASQVQKTY GVYLKEYGLQ QLHKLEAGKK LDDKPLALLY | |
| | KNSGLIASKI GEALNIEPDE VSRFASPHSL AQIFNIIEGD VAGFNKTCRA | |
| | CTYENIWRMQ EEKVESLLTN QLLSEIHGER KVPLKSAMCT RLSADSTRPF | |
| | DGQMASIIEH IARKIAQHKI AQINDVPKEF SIDIPIIIES NQFSFTAELE | |
| | EIKRGRGSAK AKKAKELGEK SKAGWVSKTE RIKTSSEGIC PYTGAPLGGS | |
| | GEIDHIIPRS LTGRTKKTVF NSEANLIYCS SKGNHDKGNR VYVIEQLNDK | |
| | YLKKQFSTSD VNLIKKKIKT TIQRFTEGGE KLRSFSELSR EDQKAFRHAL | |
| | FVPELKSEVT SLLAVKNITR VNGTQAWLAK KIASLLAEHL DKQGRDYTLS | |
| | AHQIDPWSVS KQRKMLASAE PIWAKKDPQP AASHVVDAVC TFLEALEQPH | |
| | TASRLKTISS TSFEKTGWRS ALIPDLIKVD ALDRRPKYRR YNIGSTSLFK | |
| | DGIYAERFLP ILIDENGLMA GYDIDNSLKA KGADVVFESL SPFLLFKGEE | |
| | VGAQSLSDWQ ERIDGRYLYM SIDKVKAFDY LQEKVGEKDI AAELLNSIHF | |
| | TQRKTELRAK FSDDSGKKMK TLDAIRKSLK LTVTVNEIGK RKEKCGFSGT | |
| | IGIPAKSAWE NLLDEPLLET YWGTKMPPQE IWEKVYRKHF PRNIPNQAHR | |
| | KVRKDFSLPV VDSVSGGFRV KRKTPNGYNY QLLAIDGYSA VGFKKEGDNV | |
| | DFKSPALVPQ IAESKSVTPI SSELVHLDKN EIVYFDEWRK IDISDSDLKQ | |
| | FVSSLELAPG SQNRFYIRFT VDEDQFERHF KSALRVNGIQ DLDTVNKTFD | |
| | WNREIPSLLI PPRSNLFLLE TGQKITFEYI ANGANAEVKK AYSLRRA | |
| Planococcus | MKNYTIGLDI GVASVGWVCI DENYKILNYN NRHAFGVHEF ESAESAAGRR | (SEQ |
| antarcticus | LKRGMRRRYN RRKKRLQLLQ SLFDSYITDS GFFSKTDSQH FWKNNNEFEN | ID |
| DSM 14505 | RSLTEVLSSL RISSRKYPTI YHLRSDLIES NKKMDLRLVY LALHNLVKYR | NO: |
| ANU10858.1 | GHFLQEGNWS EAASAEGMDD QLLELVTRYA ELENLSPLDL SESQWKAAET | 101) |
| | LLLNRNLTKT DQSKELTAMF GKEYEPFCKL VAGLGVSLHQ LFPSSEQALA | |
| | YKETKTKVQL SNENVEEVME LLLEEESALL EAVQPFYQQV VLYELLKGET | |
| | YVAKAKVSAF KQYQKDMASL KNLLDKTFGE KVYRSYFISD KNSQREYQKS | |
| | HKVEVLCKLD QFNKEAKFAE TFYKDLKKLL EDKSKTSIGT TEKDEMLRII | |
| | KAIDSNQFLQ KQKGIQNAAI PHQNSLYEAE KILRNQQAHY PFITTEWIEK | |
| | VKQILAFRIP YYIGPLVKDT TQSPFSWVER KGDAPITPWN FDEQIDKAAS | |
| | AEAFISRMRK TCTYLKGQEV LPKSSLTYER FEVLNELNGI QLRTTGAESD | |
| | FRHRLSYEMK CWIIDNVFKQ YKTVSTKRLL QELKKSPYAD ELYDEHTGEI | |
| | KEVFGTQKEN AFATSLSGYI SMKSILGAVV DDNPAMTEEL IYWIAVFEDR | |
| | EILHLKIQEK YPSITDVQRQ KLALVKLPGW GRFSRLLIDG LPLDEQGQSV | |
| | LDHMEQYSSV FMEVLKNKGF GLEKKIQKMN QHQVDGTKKI RYEDIEELAG | |
| | SPALKRGIWR SVKIVEELVS IFGEPANIVL EVAREDGEKK RTKSRKDQWE | |
| | ELTKTTLKND PDLKSFIGEI KSQGDQRFNE QRFWLYVTQQ GKCLYTGKAL | |
| | DIQNLSMYEV DHILPQNFVK DDSLDNLALV MPEANQRKNQ VGQNKMPLEI | |
| | IEANQQYAMR TLWERLHELK LISSGKLGRL KKPSFDEVDK DKFIARQLVE | |
| | TRQIIKHVRD LLDERFSKSD IHLVKAGIVS KFRRFSEIPK IRDYNNKHHA | |
| | MDALFAAALI QSILGKYGKN FLAFDLSKKD RQKQWRSVKG SNKEFFLFKN | |
| | FGNLRLQSPV TGEEVSGVEY MKHVYFELPW QTTKMTQTGD GMFYKESIFS | |
| | PKVKQAKYVS PKTEKFVHDE VKNHSICLVE FTFMKKEKEV QETKFIDLKV | |
| | IEHHQFLKEP ESQLAKFLAE KETNSPIIHA RIIRTIPKYQ KIWIEHFPYY | |
| | FISTRELHNA RQFEISYELM EKVKQLSERS SVEELKIVFG LLIDQMNDNY | |
| | PIYTKSSIQD RVQKFVDTQL YDFKSFEIGF EELKKAVAAN AQRSDTFGSR | |
| | ISKKPKPEEV AIGYESITGL KYRKPRSVVG TKR | |
| Prevotella sp. | MTQKVLGLDL GTNSIGSAVR NLDLSDDLQW QLEFFSSDIF RSSVNKESNG | (SEQ |
| C561 | REYSLAAQRS AHRRSRGLNE VRRRRLWATL NLLIKHGFCP MSSESLMRWC | ID |
| WP_009013303.1 | TYDKRKGLFR EYPIDDKDFN AWILLDENGD GRPDYSSPYQ LRRELVTRQF | NO: |
| | DFEQPIERYK LGRALYHIAQ HRGFKSSKGE TLSQQETNSK PSSTDEIPDV | 102) |
| | AGAMKASEEK LSKGLSTYMK EHNLLTVGAA FAQLEDEGVR VRNNNDYRAI | |
| | RSQFQHEIET IFKFQQGLSV ESELYERLIS EKKNVGTIFY KRPLRSQRGN | |
| | VGKCTLERSK PRCAIGHPLF EKFRAWTLIN NIKVRMSVDT LDEQLPMKLR | |
| | LDLYNECFLA FVRTEFKFED IRKYLEKRLG IHFSYNDKTI NYKDSTSVAG | |
| | CPITARFRKM LGEEWESFRV EGQKERQAHS KNNISFHRVS YSIEDIWHFC | |
| | YDAEEPEAVL AFAQETLRLE RKKAEELVRI WSAMPQGYAM LSQKAIRNIN | |
| | KILMLGLKYS DAVILAKVPE LVDVSDEELL SIAKDYYLVE AQVNYDKRIN | |
| | SIVIGLIAKY KSVSEEYRFA DHNYEYLLDE SDEKDIIRQI ENSLGARRWS | |
| | LMDANEQTDI LQKVRDRYQD FFRSHERKFV ESPKLGESFE NYLTKKFPMV | |
| | EREQWKKLYH PSQITIYRPV SVGKDRSVLR LGNPDIGAIK NPTVLRVLNT | |
| | LRRRVNQLLD DGVISPDETR VVVETARELN DANRKWALDT YNRIRHDENE | |
| | KIKKILEEFY PKRDGISTDD IDKARYVIDQ REVDYFTGSK TYNKDIKKYK | |
| | FWLEQGGQCM YTGRTINLSN LFDPNAFDIE HTIPESLSFD SSDMNLTLCD | |
| | AHYNRFIKKN HIPTDMPNYD KAITIDGKEY PAITSQLQRW VERVERLNRN | |
| | VEYWKGQARR AQNKDRKDQC MREMHLWKME LEYWKKKLER FTVTEVTDGF | |
| | KNSQLVDTRV ITRHAVLYLK SIFPHVDVQR GDVTAKFRKI LGIQSVDEKK | |
| | DRSLHSHHAI DATTLTIIPV SAKRDRMLEL FAKIEEINKM LSFSGSEDRT | |
| | GLIQELEGLK NKLQMEVKVC RIGHNVSEIG TFINDNIIVN HHIKNQALTP | |
| | VRRRLRKKGY IVGGVDNPRW QTGDALRGEI HKASYYGAIT QFAKDDEGKV | |
| | LMKEGRPQVN PTIKFVIRRE LKYKKSAADS GFASWDDLGK AIVDKELFAL | |
| | MKGQFPAETS FKDACEQGIY MIKKGKNGMP DIKLHHIRHV RCEAPQSGLK | |
| | IKEQTYKSEK EYKRYFYAAV GDLYAMCCYT NGKIREFRIY SLYDVSCHRK | |
| | SDIEDIPEFI TDKKGNRLML DYKLRTGDMI LLYKDNPAEL YDLDNVNLSR | |
| | RLYKINRFES QSNLVLMTHH LSTSKERGRS LGKTVDYQNL PESIRSSVKS | |
| | LNFLIMGENR DFVIKNGKII FNHR | |
| Alicyclobacillus | MAYRLGLDIG ITSVGWAVVA LEKDESGLKP VRIQDLGVRI FDKAEDSKTG | (SEQ |
| hesperidum | ASLALPRREA RSARRRTRRR RHRLWRVKRL LEQHGILSME QIEALYAQRT | ID |
| URH17-3-68 | SSPDVYALRV AGLDRCLIAE EIARVLIHIA HRRGFQSNRK SEIKDSDAGK | NO: |
| WP_006446566.1 | LLKAVQENEN LMQSKGYRTV AEMLVSEATK TDAEGKLVHG KKHGYVSNVR | 103) |
| | NKAGEYRHTV SRQAIVDEVR KIFAAQRALG NDVMSEELED SYLKILCSQR | |
| | NFDDGPGGDS PYGHGSVSPD GVRQSIYERM VGSCTFETGE KRAPRSSYSF | |
| | ERFQLLTKVV NLRIYRQQED GGRYPCELTQ TERARVIDCA YEQTKITYGK | |
| | LRKLLDMKDT ESFAGLTYGL NRSRNKTEDT VFVEMKFYHE VRKALQRAGV | |
| | FIQDLSIETL DQIGWILSVW KSDDNRRKKL STLGLSDNVI EELLPLNGSK | |
| | FGHLSLKAIR KILPFLEDGY SYDVACELAG YQFQGKTEYV KQRLLPPLGE | |
| | GEVTNPVVRR ALSQAIKVVN AVIRKHGSPE SIHIELAREL SKNLDERRKI | |
| | EKAQKENQKN NEQIKDEIRE ILGSAHVTGR DIVKYKLFKQ QQEFCMYSGE | |
| | KLDVTRLFEP GYAEVDHIIP YGISFDDSYD NKVLVKTEQN RQKGNRTPLE | |
| | YLRDKPEQKA KFIALVESIP LSQKKKNHLL MDKRAIDLEQ EGFRERNLSD | |
| | TRYITRALMN HIQAWLLFDE TASTRSKRVV CVNGAVTAYM RARWGLTKDR | |
| | DAGDKHHAAD AVVVACIGDS LIQRVTKYDK FKRNALADRN RYVQQVSKSE | |
| | GITQYVDKET GEVFTWESFD ERKFLPNEPL EPWPFFRDEL LARLSDDPSK | |
| | NIRAIGLLTY SETEQIDPIF VSRMPTRKVT GAAHKETIRS PRIVKVDDNK | |
| | GTEIQVVVSK VALTELKLTK DGEIKDYFRP EDDPRLYNTL RERLVQFGGD | |
| | AKAAFKEPVY KISKDGSVRT PVRKVKIQEK LTLGVPVHGG RGIAENGGMV | |
| | RIDVFAKGGK YYFVPIYVAD VLKRELPNRL ATAHKPYSEW RVVDDSYQFK | |
| | FSLYPNDAVM IKPSREVDIT YKDRKEPVGC RIMYFVSANI ASASISLRTH | |
| | DNSGELEGLG IQGLEVFEKY VVGPLGDTHP VYKERRMPFR VERKMN | |
| Lactobacillus | MTKLNQPYGI GLDIGSNSIG FAVVDANSHL LRLKGETAIG ARLFREGQSA | (SEQ |
| rhamnosus GG | ADRRGSRTTR RRLSRTRWRL SFLRDFFAPH ITKIDPDFFL RQKYSEISPK | ID |
| WP_014569977.1 | DKDRFKYEKR LFNDRTDAEF YEDYPSMYHL RLHLMTHTHK ADPREIFLAI | NO: |
| | HHILKSRGHF LTPGAAKDEN TDKVDLEDIF PALTEAYAQV YPDLELTFDL | 104) |
| | AKADDFKAKL LDEQATPSDT QKALVNLLLS SDGEKEIVKK RKQVLTEFAK | |
| | AITGLKTKFN LALGTEVDEA DASNWQFSMG QLDDKWSNIE TSMTDQGTEI | |
| | FEQIQELYRA RLLNGIVPAG MSLSQAKVAD YGQHKEDLEL FKTYLKKLND | |
| | HELAKTIRGL YDRYINGDDA KPFLREDFVK ALTKEVTAHP NEVSEQLLNR | |
| | MGQANFMLKQ RTKANGAIPI QLQQRELDQI IANQSKYYDW LAAPNPVEAH | |
| | RWKMPYQLDE LLNFHIPYYV GPLITPKQQA ESGENVFAWM VRKDPSGNIT | |
| | PYNFDEKVDR EASANTFIQR MKTTDTYLIG EDVLPKQSLL YQKYEVLNEL | |
| | NNVRINNECL GTDQKQRLIR EVFERHSSVT IKQVADNLVA HGDFARRPEI | |
| | RGLADEKRFL SSLSTYHQLK EILHEAIDDP TKLLDIENII TWSTVFEDHT | |
| | IFETKLAEIE WLDPKKINEL SGIRYRGWGQ FSRKLLDGLK LGNGHTVIQE | |
| | LMLSNHNLMQ ILADETLKET MTELNQDKLK TDDIEDVIND AYTSPSNKKA | |
| | LRQVLRVVED IKHAANGQDP SWLFIETADG TGTAGKRTQS RQKQIQTVYA | |
| | NAAQELIDSA VRGELEDKIA DKASFTDRLV LYFMQGGRDI YTGAPLNIDQ | |
| | LSHYDIDHIL PQSLIKDDSL DNRVLVNATI NREKNNVFAS TLFAGKMKAT | |
| | WRKWHEAGLI SGRKLRNLML RPDEIDKFAK GFVARQLVET RQIIKLTEQI | |
| | AAAQYPNTKI IAVKAGLSHQ LREELDFPKN RDVNHYHHAF DAFLAARIGT | |
| | YLLKRYPKLA PFFTYGEFAK VDVKKFREFN FIGALTHAKK NIIAKDTGEI | |
| | VWDKERDIRE LDRIYNFKRM LITHEVYFET ADLFKQTIYA AKDSKERGGS | |
| | KQLIPKKQGY PTQVYGGYTQ ESGSYNALVR VAEADTTAYQ VIKISAQNAS | |
| | KIASANLKSR EKGKQLLNEI VVKQLAKRRK NWKPSANSFK IVIPRFGMGT | |
| | LFQNAKYGLF MVNSDTYYRN YQELWLSREN QKLLKKLFSI KYEKTQMNHD | |
| | ALQVYKAIID QVEKFFKLYD INQFRAKLSD AIERFEKLPI NTDGNKIGKT | |
| | ETLRQILIGL QANGTRSNVK NLGIKTDLGL LQVGSGIKLD KDTQIVYQSP | |
| | SGLFKRRIPL ADL | |
| Enterococcus | MYSIGLDLGI SSVGWSVIDE RTGNVIDLGV RLFSAKNSEK NLERRTNRGG | (SEQ |
| faecalis | RRLIRRKTNR LKDAKKILAA VGFYEDKSLK NSCPYQLRVK GLTEPLSRGE | ID |
| TX0012 | IYKVTLHILK KRGISYLDEV DTEAAKESQD YKEQVRKNAQ LLTKYTPGQI | NO: |
| WP_002408901.1 | QLQRLKENNR VKTGINAQGN YQLNVFKVSA YANELATILK TQQAFYPNEL | 105) |
| EFT93846.1 | TDDWIALFVQ PGIAEEAGLI YRKRPYYHGP GNEANNSPYG RWSDFQKTGE | |
| | PATNIFDKLI GKDFQGELRA SGLSLSAQQY NLLNDLTNLK IDGEVPLSSE | |
| | QKEYILTELM TKEFTRFGVN DVVKLLGVKK ERLSGWRLDK KGKPEIHTLK | |
| | GYRNWRKIFA EAGIDLATLP TETIDCLAKV LTLNTEREGI ENTLAFELPE | |
| | LSESVKLLVL DRYKELSQSI STQSWHRFSL KTLHLLIPEL MNATSEQNTL | |
| | LEQFQLKSDV RKRYSEYKKL PTKDVLAEIY NPTVNKTVSQ AFKVIDALLV | |
| | KYGKEQIRYI TIEMPRDDNE EDEKKRIKEL HAKNSQRKND SQSYFMQKSG | |
| | WSQEKFQTTI QKNRRFLAKL LYYYEQDGIC AYTGLPISPE LLVSDSTEID | |
| | HIIPISISLD DSINNKVLVL SKANQVKGQQ TPYDAWMDGS FKKINGKFSN | |
| | WDDYQKWVES RHFSHKKENN LLETRNIFDS EQVEKFLARN LNDTRYASRL | |
| | VLNTLQSFFT NQETKVRVVN GSFTHTLRKK WGADLDKTRE THHHHAVDAT | |
| | LCAVTSFVKV SRYHYAVKEE TGEKVMREID FETGEIVNEM SYWEFKKSKK | |
| | YERKTYQVKW PNFREQLKPV NLHPRIKFSH QVDRKANRKL SDATIYSVRE | |
| | KTEVKTLKSG KQKITTDEYT IGKIKDIYTL DGWEAFKKKQ DKLLMKDLDE | |
| | KTYERLLSIA ETTPDFQEVE EKNGKVKRVK RSPFAVYCEE NDIPAIQKYA | |
| | KKNNGPLIRS LKYYDGKLNK HINITKDSQG RPVEKTKNGR KVTLQSLKPY | |
| | RYDIYQDLET KAYYTVQLYY SDLRFVEGKY GITEKEYMKK VAEQTKGQVV | |
| | RFCFSLQKND GLEIEWKDSQ RYDVRFYNFQ SANSINFKGL EQEMMPAENQ | |
| | FKQKPYNNGA INLNIAKYGK EGKKLRKFNT DILGKKHYLF YEKEPKNIIK | |
| Candidatus | MRRLGLDLGT NSIGWCLLDL GDDGEPVSIF RTGARIFSDG RDPKSLGSLK | (SEQ |
| Puniceispirillum | ATRREARLTR RRRDRFIQRQ KNLINALVKY GLMPADEIQR QALAYKDPYP | ID |
| marinum | IRKKALDEAI DPYEMGRAIF HINQRRGFKS NRKSADNEAG VVKQSIADLE | NO: |
| IMCC1322 | MKLGEAGART IGEFLADRQA TNDTVRARRL SGTNALYEFY PDRYMLEQEF | 106) |
| WP_013047413.1 | DTLWAKQAAF NPSLYIEAAR ERLKEIVFFQ RKLKPQEVGR CIFLSDEDRI | |
| | SKALPSFQRF RIYQELSNLA WIDHDGVAHR ITASLALRDH LFDELEHKKK | |
| | LTFKAMRAIL RKQGVVDYPV GFNLESDNRD HLIGNLTSCI MRDAKKMIGS | |
| | AWDRLDEEEQ DSFILMLQDD QKGDDEVRSI LTQQYGLSDD VAEDCLDVRL | |
| | PDGHGSLSKK AIDRILPVLR DQGLIYYDAV KEAGLGEANL YDPYAALSDK | |
| | LDYYGKALAG HVMGASGKFE DSDEKRYGTI SNPTVHIALN QVRAVVNELI | |
| | RLHGKPDEVV IEIGRDLPMG ADGKRELERF QKEGRAKNER ARDELKKLGH | |
| | IDSRESRQKF QLWEQLAKEP VDRCCPFTGK MMSISDLFSD KVEIEHLLPF | |
| | SLTLDDSMAN KTVCFRQANR DKGNRAPFDA FGNSPAGYDW QEILGRSQNL | |
| | PYAKRWRFLP DAMKRFEADG GFLERQLNDT RYISRYTTEY ISTIIPKNKI | |
| | WVVTGRLTSL LRGFWGLNSI LRGHNTDDGT PAKKSRDDHR HHAIDAIVVG | |
| | MTSRGLLQKV SKAARRSEDL DLTRLFEGRI DPWDGFRDEV KKHIDAIIVS | |
| | HRPRKKSQGA LHNDTAYGIV EHAENGASTV VHRVPITSLG KQSDIEKVRD | |
| | PLIKSALLNE TAGLSGKSFE NAVQKWCADN SIKSLRIVET VSIIPITDKE | |
| | GVAYKGYKGD GNAYMDIYQD PTSSKWKGEI VSRFDANQKG FIPSWQSQFP | |
| | TARLIMRLRI NDLLKLQDGE IEEIYRVQRL SGSKILMAPH TEANVDARDR | |
| | DKNDTFKLTS KSPGKLQSAS ARKVHISPTG LIREG | |
| Oenococcus | MARDYSVGLD IGTSSVGWAA IDNKYHLIRA KSKNLIGVRL FDSAVTAEKR | (SEQ |
| kitaharae DSM | RGYRTTRRRL SRRHWRLRLL NDIFAGPLTD FGDENFLARL KYSWVHPQDQ | ID |
| 17330 | SNQAHFAAGL LFDSKEQDKD FYRKYPTIYH LRLALMNDDQ KHDLREVYLA | NO: |
| EHN59352.1 | IHHLVKYRGH FLIEGDVKAD SAFDVHTFAD AIQRYAESNN SDENLLGKID | 107) |
| | EKKLSAALTD KHGSKSQRAE TAETAFDILD LQSKKQIQAI LKSVVGNQAN | |
| | LMAIFGLDSS AISKDEQKNY KFSFDDADID EKIADSEALL SDTEFEFLCD | |
| | LKAAFDGLTL KMLLGDDKTV SAAMVRRFNE HQKDWEYIKS HIRNAKNAGN | |
| | GLYEKSKKFD GINAAYLALQ SDNEDDRKKA KKIFQDEISS ADIPDDVKAD | |
| | FLKKIDDDQF LPIQRTKNNG TIPHQLHRNE LEQIIEKQGI YYPFLKDTYQ | |
| | ENSHELNKIT ALINFRVPYY VGPLVEEEQK IADDGKNIPD PTNHWMVRKS | |
| | NDTITPWNLS QVVDLDKSGR RFIERLTGTD TYLIGEPTLP KNSLLYQKFD | |
| | VLQELNNIRV SGRRLDIRAK QDAFEHLFKV QKTVSATNLK DFLVQAGYIS | |
| | EDTQIEGLAD VNGKNFNNAL TTYNYLVSVL GREFVENPSN EELLEEITEL | |
| | QTVFEDKKVL RRQLDQLDGL SDHNREKLSR KHYTGWGRIS KKLLTTKIVQ | |
| | NADKIDNQTF DVPRMNQSII DTLYNTKMNL MEIINNAEDD FGVRAWIDKQ | |
| | NTTDGDEQDV YSLIDELAGP KEIKRGIVQS FRILDDITKA VGYAPKRVYL | |
| | EFARKTQESH LTNSRKNQLS TLLKNAGLSE LVTQVSQYDA AALQNDRLYL | |
| | YFLQQGKDMY SGEKLNLDNL SNYDIDHIIP QAYTKDNSLD NRVLVSNITN | |
| | RRKSDSSNYL PALIDKMRPF WSVLSKQGLL SKHKFANLTR TRDFDDMEKE | |
| | RFIARSLVET RQIIKNVASL IDSHFGGETK AVAIRSSLTA DMRRYVDIPK | |
| | NRDINDYHHA FDALLFSTVG QYTENSGLMK KGQLSDSAGN QYNRYIKEWI | |
| | HAARLNAQSQ RVNPFGFVVG SMRNAAPGKL NPETGEITPE ENADWSIADL | |
| | DYLHKVMNFR KITVTRRLKD QKGQLYDESR YPSVLHDAKS KASINFDKHK | |
| | PVDLYGGFSS AKPAYAALIK FKNKFRLVNV LRQWTYSDKN SEDYILEQIR | |
| | GKYPKAEMVL SHIPYGQLVK KDGALVTISS ATELHNFEQL WLPLADYKLI | |
| | NTLLKTKEDN LVDILHNRLD LPEMTIESAF YKAFDSILSF AFNRYALHQN | |
| | ALVKLQAHRD DFNALNYEDK QQTLERILDA LHASPASSDL KKINLSSGFG | |
| | RLFSPSHFTL ADTDEFIFQS VTGLFSTQKT VAQLYQETK | |
| Helicobacter | MIRTLGIDIG IASIGWAVIE GEYTDKGLEN KEIVASGVRV FTKAENPKNK | (SEQ |
| mustelae | ESLALPRTLA RSARRRNARK KGRIQQVKHY LSKALGLDLE CFVQGEKLAT | ID |
| 12198 | LFQTSKDFLS PWELRERALY RVLDKEELAR VILHIAKRRG YDDITYGVED | NO: |
| WP_013022389.1 | NDSGKIKKAI AENSKRIKEE QCKTIGEMMY KLYFQKSLNV RNKKESYNRC | 108) |
| | VGRSELREEL KTIFQIQQEL KSPWVNEELI YKLLGNPDAQ SKQEREGLIF | |
| | YQRPLKGFGD KIGKCSHIKK GENSPYRACK HAPSAEEFVA LTKSINFLKN | |
| | LTNRHGLCFS QEDMCVYLGK ILQEAQKNEK GLTYSKLKLL LDLPSDFEFL | |
| | GLDYSGKNPE KAVFLSLPST FKLNKITQDR KTQDKIANIL GANKDWEAIL | |
| | KELESLQLSK EQIQTIKDAK LNFSKHINLS LEALYHLLPL MREGKRYDEG | |
| | VEILQERGIF SKPQPKNRQL LPPLSELAKE ESYFDIPNPV LRRALSEFRK | |
| | VVNALLEKYG GFHYFHIELT RDVCKAKSAR MQLEKINKKN KSENDAASQL | |
| | LEVLGLPNTY NNRLKCKLWK QQEEYCLYSG EKITIDHLKD QRALQIDHAF | |
| | PLSRSLDDSQ SNKVLCLTSS NQEKSNKTPY EWLGSDEKKW DMYVGRVYSS | |
| | NFSPSKKRKL TQKNFKERNE EDFLARNLVD TGYIGRVTKE YIKHSLSFLP | |
| | LPDGKKEHIR IISGSMTSTM RSFWGVQEKN RDHHLHHAQD AIIIACIEPS | |
| | MIQKYTTYLK DKETHRLKSH QKAQILREGD HKLSLRWPMS NFKDKIQESI | |
| | QNIIPSHHVS HKVTGELHQE TVRTKEFYYQ AFGGEEGVKK ALKFGKIREI | |
| | NQGIVDNGAM VRVDIFKSKD KGKFYAVPIY TYDFAIGKLP NKAIVQGKKN | |
| | GIIKDWLEMD ENYEFCFSLF KNDCIKIQTK EMQEAVLAIY KSTNSAKATI | |
| | ELEHLSKYAL KNEDEEKMFT DTDKEKNKTM TRESCGIQGL KVFQKVKLSV | |
| | LGEVLEHKPR NRQNIALKTT PKHV | |
| Bradyrhizobium | MKRTSLRAYR LGVDLGANSL GWFVVWLDDH GQPEGLGPGG VRIFPDGRNP | (SEQ |
| sp. BTAil | QSKQSNAAGR RLARSARRRR DRYLQRRGKL MGLLVKHGLM PADEPARKRL | ID |
| WP_012044026.1 | ECLDPYGLRA KALDEVLPLH HVGRALFHLN QRRGLFANRA IEQGDKDASA | NO: |
| | IKAAAGRLQT SMQACGARTL GEFLNRRHQL RATVRARSPV GGDVQARYEF | 109) |
| | YPTRAMVDAE FEAIWAAQAP HHPTMTAEAH DTIREAIFSQ RAMKRPSIGK | |
| | CSLDPATSQD DVDGFRCAWS HPLAQRFRIW QDVRNLAVVE TGPTSSRLGK | |
| | EDQDKVARAL LQTDQLSFDE IRGLLGLPSD ARFNLESDRR DHLKGDATGA | |
| | ILSARRHFGP AWHDRSLDRQ IDIVALLESA LDEAAIIASL GTTHSLDEAA | |
| | AQRALSALLP DGYCRLGLRA IKRVLPLMEA GRTYAEAASA AGYDHALLPG | |
| | GKLSPTGYLP YYGQWLQNDV VGSDDERDTN ERRWGRLPNP TVHIGIGQLR | |
| | RVVNELIRWH GPPAEITVEL TRDLKLSPRR LAELEREQAE NQRKNDKRTS | |
| | LLRKLGLPAS THNLLKLRLW DEQGDVASEC PYTGEAIGLE RLVSDDVDID | |
| | HLIPFSISWD DSAANKVVCM RYANREKGNR TPFEAFGHRQ GRPYDWADIA | |
| | ERAARLPRGK RWRFGPGARA QFEELGDFQA RLLNETSWLA RVAKQYLAAV | |
| | THPHRIHVLP GRLTALLRAT WELNDLLPGS DDRAAKSRKD HRHHAIDALV | |
| | AALTDQALLR RMANAHDDTR RKIEVLLPWP_TFRIDLETRL KAMLVSHKPD | |
| | HGLQARLHED TAYGTVEHPE TEDGANLVYR KTFVDISEKE IDRIRDRRLR | |
| | DLVRAHVAGE RQQGKTLKAA VLSFAQRRDI AGHPNGIRHV RLTKSIKPDY | |
| | LVPIRDKAGR IYKSYNAGEN AFVDILQAES GRWIARATTV FQANQANESH | |
| | DAPAAQPIMR VFKGDMLRID HAGAEKFVKI VRLSPSNNLL YLVEHHQAGV | |
| | FQTRHDDPED SFRWLFASFD KLREWNAELV RIDTLGQPWR RKRGLETGSE | |
| | DATRIGWTRP KKWP | |
| Acidaminococcus | MGKMYYLGLD IGTNSVGYAV TDPSYHLLKF KGEPMWGAHV FAAGNQSAER | (SEQ |
| sp. D21 | RSFRTSRRRL DRRQQRVKLV QEIFAPVISP IDPRFFIRLH ESALWRDDVA | ID |
| WP_009016219.1 | ETDKHIFFND PTYTDKEYYS DYPTIHHLIV DLMESSEKHD PRLVYLAVAW | NO: |
| | LVAHRGHFLN EVDKDNIGDV LSFDAFYPEF LAFLSDNGVS PWVCESKALQ | 110) |
| | ATLLSRNSVN DKYKALKSLI FGSQKPEDNF DANISEDGLI QLLAGKKVKV | |
| | NKLFPQESND ASFTLNDKED AIEEILGTLT PDECEWIAHI RRLFDWAIMK | |
| | HALKDGRTIS ESKVKLYEQH HHDLTQLKYF VKTYLAKEYD DIFRNVDSET | |
| | TKNYVAYSYH VKEVKGTLPK NKATQEEFCK YVLGKVKNIE CSEADKVDFD | |
| | EMIQRLTDNS FMPKQVSGEN RVIPYQLYYY ELKTILNKAA SYLPFLTQCG | |
| | KDAISNQDKL LSIMTFRIPY FVGPLRKDNS EHAWLERKAG KIYPWNFNDK | |
| | VDLDKSEEAF IRRMTNTCTY YPGEDVLPLD SLIYEKFMIL NEINNIRIDG | |
| | YPISVDVKQQ VFGLFEKKRR VTVKDIQNLL LSLGALDKHG KLTGIDTTIH | |
| | SNYNTYHHFK SLMERGVLTR DDVERIVERM TYSDDTKRVR LWLNNNYGTL | |
| | TADDVKHISR LRKHDFGRLS KMFLTGLKGV HKETGERASI LDFMWNTNDN | |
| | LMQLLSECYT FSDEITKLQE AYYAKAQLSL NDFLDSMYIS NAVKRPIYRT | |
| | LAVVNDIRKA CGTAPKRIFI EMARDGESKK KRSVTRREQI KNLYRSIRKD | |
| | FQQEVDFLEK ILENKSDGQL QSDALYLYFA QLGRDMYTGD PIKLEHIKDQ | |
| | SFYNIDHIYP QSMVKDDSLD NKVLVQSEIN GEKSSRYPLD AAIRNKMKPL | |
| | WDAYYNHGLI SLKKYQRLTR STPFTDDEKW DFINRQLVET RQSTKALAIL | |
| | LKRKFPDTEI VYSKAGLSSD FRHEFGLVKS RNINDLHHAK DAFLAIVTGN | |
| | VYHERFNRRW FMVNQPYSVK TKTLFTHSIK NGNFVAWNGE EDLGRIVKML | |
| | KQNKNTIHFT RFSFDRKEGL FDIQPLKAST GLVPRKAGLD VVKYGGYDKS | |
| | TAAYYLLVRF TLEDKKTQHK LMMIPVEGLY KARIDHDKEF LTDYAQTTIS | |
| | EILQKDKQKV INIMFPMGTR HIKLNSMISI DGFYLSIGGK SSKGKSVLCH | |
| | AMVPLIVPHK IECYIKAMES FARKFKENNK LRIVEKFDKI TVEDNLNLYE | |
| | LFLQKLQHNP YNKFFSTQFD VLTNGRSTFT KLSPEEQVQT LLNILSIFKT | |
| | CRSSGCDLKS INGSAQAARI MISADLTGLS KKYSDIRLVE QSASGLFVSK | |
| | SQNLLEYL | |
| Methylosinus | MRVLGLDAGI ASLGWALIEI EESNRGELSQ GTIIGAGTWM FDAPEEKTQA | (SEQ |
| trichosporium | GAKLKSEQRR TFRGQRRVVR RRRQRMNEVR RILHSHGLLP SSDRDALKQP | ID |
| OB3b | GLDPWRIRAE ALDRLLGPVE LAVALGHIAR HRGFKSNSKG AKTNDPADDT | NO: |
| WP_003611034.1 | SKMKRAVNET REKLARFGSA AKMLVEDESF VLRQTPTKNG ASEIVRRFRN | 111) |
| | REGDYSRSLL RDDLAAEMRA LFTAQARFQS AIATADLQTA FTKAAFFQRP | |
| | LQDSEKLVGP CPFEVDEKRA PKRGYSFELF RFLSRLNHVT LRDGKQERTL | |
| | TRDELALAAA DFGAAAKVSF TALRKKLKLP ETTVFVGVKA DEESKLDVVA | |
| | RSGKAAEGTA RLRSVIVDAL GELAWGALLC SPEKLDKIAE VISFRSDIGR | |
| | ISEGLAQAGC NAPLVDALTA AASDGRFDPF TGAGHISSKA ARNILSGLRQ | |
| | GMTYDKACCA ADYDHTASRE RGAFDVGGHG REALKRILQE ERISRELVGS | |
| | PTARKALIES IKQVKAIVER YGVPDRIHVE LARDVGKSIE EREEITRGIE | |
| | KRNRQKDKLR GLFEKEVGRP PQDGARGKEE LLRFELWSEQ MGRCLYTDDY | |
| | ISPSQLVATD DAVQVDHILP WSRFADDSYA NKTLCMAKAN QDKKGRTPYE | |
| | WFKAEKTDTE WDAFIVRVEA LADMKGFKKR NYKLRNAEEA AAKFRNRNLN | |
| | DTRWACRLLA EALKQLYPKG EKDKDGKERR RVFSRPGALT DRLRRAWGLQ | |
| | WMKKSTKGDR IPDDRHHALD AIVIAATTES LLQRATREVQ EIEDKGLHYD | |
| | LVKNVTPPWP_GFREQAVEAV EKVFVARAER RRARGKAHDA TIRHIAVREG | |
| | EQRVYERRKV AELKLADLDR VKDAERNARL IEKLRNWIEA GSPKDDPPLS | |
| | PKGDPIFKVR LVTKSKVNIA LDTGNPKRPG TVDRGEMARV DVFRKASKKG | |
| | KYEYYLVPIY PHDIATMKTP PIRAVQAYKP EDEWPEMDSS YEFCWSLVPM | |
| | TYLQVISSKG EIFEGYYRGM NRSVGAIQLS AHSNSSDVVQ GIGARTLTEF | |
| | KKFNVDRFGR KHEVERELRT WRGETWRGKA YI | |
| Actinomyces | MDNKNYRIGI DVGLNSIGFC AVEVDQHDTP LGFLNLSVYR HDAGIDPNGK | (SEQ |
| coleocanis | KTNTTRLAMS GVARRTRRLF RKRKRRLAAL DRFIEAQGWT LPDHADYKDP | ID |
| DSM 15436 | YTPWLVRAEL AQTPIRDEND LHEKLAIAVR HIARHRGWRS PWVPVRSLHV | NO: |
| WP_006546479.1 | EQPPSDQYLA LKERVEAKTL LQMPEGATPA EMVVALDLSV DVNLRPKNRE | 112) |
| | KTDTRPENKK PGFLGGKLMQ SDNANELRKI AKIQGLDDAL LRELIELVFA | |
| | ADSPKGASGE LVGYDVLPGQ HGKRRAEKAH PAFQRYRIAS IVSNLRIRHL | |
| | GSGADERLDV ETQKRVFEYL LNAKPTADIT WSDVAEEIGV ERNLLMGTAT | |
| | QTADGERASA KPPVDVTNVA FATCKIKPLK EWWLNADYEA RCVMVSALSH | |
| | AEKLTEGTAA EVEVAEFLQN LSDEDNEKLD SFSLPIGRAA YSVDSLERLT | |
| | KRMIENGEDL FEARVNEFGV SEDWRPPAEP IGARVGNPAV DRVLKAVNRY | |
| | LMAAEAEWGA PLSVNIEHVR EGFISKRQAV EIDRENQKRY QRNQAVRSQI | |
| | ADHINATSGV RGSDVTRYLA IQRQNGECLY CGTAITFVNS EMDHIVPRAG | |
| | LGSTNTRDNL VATCERCNKS KSNKPFAVWA AECGIPGVSV AEALKRVDFW | |
| | IADGFASSKE HRELQKGVKD RLKRKVSDPE IDNRSMESVA WMARELAHRV | |
| | QYYFDEKHTG TKVRVFRGSL TSAARKASGF ESRVNFIGGN GKTRLDRRHH | |
| | AMDAATVAML RNSVAKTLVL RGNIRASERA IGAAETWKSF RGENVADRQI | |
| | FESWSENMRV LVEKFNLALY NDEVSIFSSL RLQLGNGKAH DDTITKLQMH | |
| | KVGDAWSLTE IDRASTPALW CALTRQPDFT WKDGLPANED RTIIVNGTHY | |
| | GPLDKVGIFG KAAASLLVRG GSVDIGSAIH HARIYRIAGK KPTYGMVRVF | |
| | APDLLRYRNE DLFNVELPPQ SVSMRYAEPK VREAIREGKA EYLGWLVVGD | |
| | GEDVSEGSKS IIAGQGWRPA VNKVFGSAMP EVIRRDGLGR KRRFSYSGLP | |
| | VSWQG | |
| | ELLLDLSSET SGQIAELQQD FPGTTHWTVA GFFSPSRLRL RPVYLAQEGL | (SEQ |
| Caenispirillum | MPVLSPLSPN AAQGRRRWSL ALDIGEGSIG WAVAEVDAEG RVLQLTGTGV | ID |
| salinarum AK4 | TLFPSAWSNE NGTYVAHGAA DRAVRGQQQR HDSRRRRLAG LARLCAPVLE | NO: |
| WP_009541330.1 | RSPEDLKDLT RTPPKADPRA IFFLRADAAR RPLDGPELFR VLHHMAAHRG | 113) |
| | IRLAELQEVD PPPESDADDA APAATEDEDG TRRAAADERA FRRLMAEHMH | |
| | RHGTQPTCGE IMAGRLRETP AGAQPVTRAR DGLRVGGGVA VPTRALIEQE | |
| | FDAIRAIQAP RHPDLPWDSL RRLVLDQAPI AVPPATPCLF LEELRRRGET | |
| | FQGRTITREA IDRGLTVDPL IQALRIRETV GNLRLHERIT EPDGRQRYVP | |
| | RAMPELGLSH GELTAPERDT LVRALMHDPD GLAAKDGRIP YTRLRKLIGY | |
| | DNSPVCFAQE RDTSGGGITV NPTDPLMARW IDGWVDLPLK ARSLYVRDVV | |
| | ARGADSAALA RLLAEGAHGV PPVAAAAVPA ATAAILESDI MQPGRYSVCP | |
| | WAAEAILDAW ANAPTEGFYD VTRGLFGFAP GEIVLEDLRR ARGALLAHLP | |
| | RTMAAARTPN RAAQQRGPLP AYESVIPSQL ITSLRRAHKG RAADWSAADP | |
| | EERNPFLRTW TGNAATDHIL NQVRKTANEV ITKYGNRRGW DPLPSRITVE | |
| | LAREAKHGVI RRNEIAKENR ENEGRRKKES AALDTFCQDN TVSWQAGGLP | |
| | KERAALRLRL AQRQEFFCPY CAERPKLRAT DLFSPAETEI DHVIERRMGG | |
| | DGPDNLVLAH KDCNNAKGKK TPHEHAGDLL DSPALAALWQ GWRKENADRL | |
| | KGKGHKARTP REDKDFMDRV GWRFEEDARA KAEENQERRG RRMLHDTARA | |
| | TRLARLYLAA AVMPEDPAEI GAPPVETPPS PEDPTGYTAI YRTISRVQPV | |
| | NGSVTHMLRQ RLLQRDKNRD YQTHHAEDAC LLLLAGPAVV QAFNTEAAQH | |
| | GADAPDDRPV DLMPTSDAYH QQRRARALGR VPLATVDAAL ADIVMPESDR | |
| | ATHYGRREIT VDGRTDTVVT QRMNARDLVA LLDNAKIVPA ARLDAAAPGD | |
| | QDPETGRVHW RLTRAGRGLK RRIDDLTRNC VILSRPRRPS ETGTPGALHN | |
| | TILKEICTEI ADRHDRVVDP EGTHARRWIS ARLAALVPAH AEAVARDIAE | |
| | LADLDALADA DRTPEQEARR SALRQSPYLG RAISAKKADG RARAREQEIL | |
| | TRALLDPHWG PRGLRHLIMR EARAPSLVRI RANKTDAFGR PVPDAAVWVK | |
| | TDGNAVSQLW RLTSVVTDDG RRIPLPKPIE KRIEISNLEY ARLNGLDEGA | |
| | GVTGNNAPPR PLRQDIDRLT PLWRDHGTAP GGYLGTAVGE LEDKARSALR | |
| | GKAMRQTLTD AGITAEAGWR LDSEGAVCDL EVAKGDTVKK DGKTYKVGVI | |
| | TQGIFGMPVD AAGSAPRTPE DCEKFEEQYG IKPWKAKGIP LA | |
| Coriobacterium | MKLRGIEDDY SIGLDMGTSS VGWAVTDERG TLAHFKRKPT WGSRLFREAQ | (SEQ |
| glomerans | TAAVARMPRG QRRRYVRRRW RLDLLQKLFE QQMEQADPDF FIRLRQSRLL | ID |
| PW2 | RDDRAEEHAD YRWPLFNDCK FTERDYYQRF PTIYHVRSWL METDEQADIR | NO: |
| WP_013709575.1 | LIYLALHNIV KHRGNFLREG QSLSAKSARP DEALNHLRET LRVWSSERGF | 114) |
| | ECSIADNGSI LAMLTHPDLS PSDRRKKIAP LFDVKSDDAA ADKKLGIALA | |
| | GAVIGLKTEF KNIFGDFPCE DSSIYLSNDE AVDAVRSACP DDCAELFDRL | |
| | CEVYSAYVLQ GLLSYAPGQT ISANMVEKYR RYGEDLALLK KLVKIYAPDQ | |
| | YRMFFSGATY PGTGIYDAAQ ARGYTKYNLG PKKSEYKPSE SMQYDDFRKA | |
| | VEKLFAKTDA RADERYRMMM DRFDKQQFLR RLKTSDNGSI YHQLHLEELK | |
| | AIVENQGRFY PFLKRDADKL VSLVSFRIPY YVGPLSTRNA RTDQHGENRF | |
| | AWSERKPGMQ DEPIFPWNWE SIIDRSKSAE KFILRMTGMC TYLQQEPVLP | |
| | KSSLLYEEFC VLNELNGAHW SIDGDDEHRF DAADREGIIE ELFRRKRTVS | |
| | YGDVAGWMER ERNQIGAHVC GGQGEKGFES KLGSYIFFCK DVFKVERLEQ | |
| | SDYPMIERII LWNTLFEDRK ILSQRLKEEY GSRLSAEQIK TICKKRFTGW | |
| | GRLSEKFLTG ITVQVDEDSV SIMDVLREGC PVSGKRGRAM VMMEILRDEE | |
| | LGFQKKVDDF NRAFFAENAQ ALGVNELPGS PAVRRSLNQS IRIVDEIASI | |
| | AGKAPANIFI EVTRDEDPKK KGRRTKRRYN DLKDALEAFK KEDPELWREL | |
| | CETAPNDMDE RLSLYFMQRG KCLYSGRAID IHQLSNAGIY EVDHIIPRTY | |
| | VKDDSLENKA LVYREENQRK TDMLLIDPEI RRRMSGYWRM LHEAKLIGDK | |
| | KFRNLLRSRI DDKALKGFIA RQLVETGQMV KLVRSLLEAR YPETNIISVK | |
| | ASISHDLRTA AELVKCREAN DFHHAHDAFL ACRVGLFIQK RHPCVYENPI | |
| | GLSQVVRNYV RQQADIFKRC RTIPGSSGFI VNSFMTSGFD KETGEIFKDD | |
| | WDAEAEVEGI RRSLNFRQCF ISRMPFEDHG VFWDATIYSP RAKKTAALPL | |
| | KQGLNPSRYG SFSREQFAYF FIYKARNPRK EQTLFEFAQV PVRLSAQIRQ | |
| | DENALERYAR ELAKDQGLEF IRIERSKILK NQLIEIDGDR LCITGKEEVR | |
| | NACELAFAQD EMRVIRMLVS EKPVSRECVI SLFNRILLHG DQASRRLSKQ | |
| | LKLALLSEAF SEASDNVQRN VVLGLIAIFN GSTNMVNLSD IGGSKFAGNV | |
| | RIKYKKELAS PKVNVHLIDQ SVTGMFERRT KIGL | |

In some embodiments, prime editors utilized herein comprise CRISPR-Cas system enzymes other than type II enzymes. In certain embodiments, prime editors comprise type V or type VI CRISPR-Cas system enzymes. It will be appreciated that certain CRISPR enzymes exhibit promiscuous ssDNA cleavage activity and appropriate precautions should be considered. In certain embodiments, prime editors comprise a nickase or a dead CRISPR with nuclease function comprised in a different component.

In various embodiments, the nucleic acid programmable DNA binding proteins utilized herein include, without limitation, Cas9 (e.g., dCas9 and nCas9), Cas12a (Cpf1), Cas12b1 (C2cl), Cas12b2, Cas12c (C2c3), Cas12d (CasY), Cas12e (CasX), C2c4, C2c5, C2c8, C2c9, C2c10, Cas13a (C2c2), Cas13b (C2c6), Cas13c (C2c7), Cas13d, and Argonaute. Cas-equivalents further include those described in Makarova et al., “C2c2 is a single-component programmable RNA-guided RNA-targeting CRISPR effector,” Science 2016; 353 (6299) and Makarova et al., “Classification and Nomenclature of CRISPR-Cas Systems: Where from Here?,” The CRISPR Journal, Vol. 1. No. 5, 2018, the contents of which are incorporated herein by reference. One example of a nucleic acid programmable DNA-binding protein that has different PAM specificity than Cas9 is Clustered Regularly Interspaced Short Palindromic Repeats from Prevotella and Francisella 1 (i.e, Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

### 6.3. Type V CRISPR Proteins

In some embodiments, prime editors used herein comprise the type V CRISPR family includes Francisella novicida (112 Cpf1 (FnCpf1) also known as FnCas12a. FnCpf1 adopts a bilobed architecture with the two lobes connected by the wedge (WED) domain. The N-terminal REC lobe consists of two a-helical domains (REC1 and REC2) that have been shown to coordinate the crRNA-target DNA heteroduplex. The C-terminal NUC lobe consists of the C-terminal RuvC and Nuc domains involved in target cleavage, the arginine-rich bridge helix (BH), and the PAM-interacting (PI) domain. The repeat-derived segment of the crRNA forms a pseudoknot stabilized by intra-molecular base-pairing and hydrogen-bonding interactions. The pseudoknot is coordinated by residues from the WED, RuvC, and REC2 domains, as well as by two hydrated magnesium cations. Notably, nucleotides 1-5 of the crRNA are ordered in the central cavity of FnCas12a and adopt an A-form-like helical conformation. Conformational ordering of the seed sequence is facilitated by multiple interactions between the ribose and phosphate moieties of the crRNA backbone and FnCpf1 residues in the WED and REC1 domains. These include residues Thr16, Lys595, His804, and His881 from the WED domain and residues Tyr47, Lys51, Phe182, and Arg 186 from the REC1 domain. The structure of the FnCas12a-crRNA complex further reveals that the bases of the seed sequence are solvent exposed and poised for hybridization with target DNA. Structural aspects of FnCpf1 are described by Swarts et al., Structural Basis for Guide RNA Processing and Seed-Dependent DNA Targeting by CRISPR-Cas12a, Molecular Cell 66, 221-233, Apr. 20, 2017.

Pre-crRNA processing: Essential residues for crRNA processing include His843, Lys852, and Lys869. Structural observations are consistent with an acid-base catalytic mechanism in which Lys869 acts as the general base catalyst to deprotonate the attacking 2′-hydroxyl group of U (−19), while His843 acts as a general acid to protonate the 5′-oxygen leaving group of A (−18). In turn, the side chain of Lys852 is involved in charge stabilization of the transition state. Collectively, these interactions facilitate the intra-molecular attack of the 20-hydroxyl group of U (−19) on the scissile phosphate and promote the formation of the 2′,3′-cyclic phosphate product.

R-loop formation: The crRNA-target DNA strand heteroduplex is enclosed in the central cavity formed by the REC and NUC lobes and interacts extensively with the REC1 and REC2 domains. The PAM-containing DNA duplex comprises target strand nucleotides dTO-dT8 and non-target strand nucleotides dA (8)*dAO* and is contacted by the PI, WED, and REC1 domains. The 5′-TTN-3′ PAM is recognized in FnCas12a by a mechanism combining the shape-specific recognition of a narrowed minor groove, with base-specific recognition of the PAM bases by two invariant residues, Lys671 and Lys613. Directly downstream of the PAM, the duplex of the target DNA is disrupted by the side chain of residue Lys667, which is inserted between the DNA strands and forms a cation-x stacking interaction with the dAO-dTO* base pair. The phosphate group linking target strand residues dT (−1) and dTO is coordinated by hydrogen-bonding interactions with the side chain of Lys823 and the backbone amide of Gly826. Target strand residue dT (−1) bends away from residue TO, allowing the target strand to interact with the seed sequence of the crRNA. The non-target strand nucleotides dT1*-dT5* interact with the Arg692-Ser702 loop in FnCas 12a through hydrogen-bonding and ionic interactions between backbone phosphate groups and side chains of Arg692, Asn700, Ser702, and Gln704, as well as main-chain amide groups of Lys699, Asn700, and Ser702. Alanine substitution of Q704 or replacement of residues Thr698-Ser702 in FnCas12a with the sequence Ala-Gly3 (SEQ ID NO: 115) substantially reduced DNA cleavage activity, suggesting that these residues contribute to R-loop formation by stabilizing the displaced conformation of the nontarget DNA strand.

In the FnCas12a R-loop complex, the crRNA-target strand heteroduplex is terminated by a stacking interaction with a conserved aromatic residue (Tyr410). This prevents base pairing between the crRNA and the target strand beyond nucleotides U20 and dA (−20), respectively. Beyond this point, the target DNA strand nucleotides re-engage the non-target DNA strand, forming a PAM-distal DNA duplex comprising nucleotides dC (−21)-dA (−27) and dG21*-dT27*, respectively. The duplex is confined between the REC2 and Nuc domains at the end of the central channel formed by the REC and NUC lobes.

Target DNA cleavage: FnCpf1 can independently accommodate both the target and non-target DNA strands in the catalytic pocket of the RuvC domain. The RuvC active site contains three catalytic residues (D917, E1006, and D1255). Structural observations suggest that both the target and non-target DNA strands are cleaved by the same catalytic mechanism in a single active site in Cpf1/Cas12a enzymes.

Another type V CRISPR is AsCpf1 from Acidaminococcus sp BV31.6 (Yamano et al., Crystal structure of Cpf1 in complex with guide RNA and target DNA, Cell 165, 949-962, May 5, 2016)

In certain embodiments, the nuclease comprises a Cas 12f effector. Small CRISPR-associated effector proteins belonging to the type V-F subtype have been identified through the mining of sequence databases and members classified into Cas12f1 (Cas14a and type V-U3), Cas12f2 (Cas14b) and Cas12f3 (Cas14c, type V-U2 and U4). (See, e.g., Karvelis et al., PAM recognition by miniature CRISPR-Cas12f nucleases triggers programmable double-stranded DNA target cleavage. Nucleic Acids Research, 21 May 2020, 48 (9), 5016-23 doi.org/10.1093/nar/gkaa208). Xu et al. described development of a 529 amino acid Cas12f-based system for mammalian genome engineering through multiple rounds of iterative protein engineering and screening. (Xu, X. et al., Engineered Miniature CRISPR-Cas System for Mammalian Genome Regulation and Editing. Molecular Cell, Oct. 21, 2021, 81 (20): 4333-45, doi.org/10.1016/j.molcel. 2021.08.008).

Exemplary CRISPR-Cas proteins and enzymes used in the Prime Editors herein include the following without limitation.

TABLE 5
Cas12a orthologs

| | | |
|---|---|---|
| KKP36646_(modified) | MSNFFKNFTN LYELSKTLRF ELKPVGDTLT NMKDHLEYDE KLQTFLKDQN | (SEQ ID |
| hypothetical | IDDAYQALKP QFDEIHEEFI TDSLESKKAK EIDFSEYLDL FQEKKELNDS | NO: 116) |
| protein | EKKLRNKIGE TFNKAGEKWK KEKYPQYEWK KGSKIANGAD ILSCQDMLQF | |
| UR27 C0015G0004 | IKYKNPEDEK IKNYIDDTLK GFFTYFGGFN QNRANYYETK KEASTAVATR | |
| [Candidatus | IVHENLPKFC DNVIQFKHII KRKKDGTVEK TERKTEYLNA YQYLKNNNKI | |
| Peregrinibacteria | TQIKDAETEK MIESTPIAEK IFDVYYFSSC LSQKQIEEYN RIIGHYNLLI | |
| bacterium | NLYNQAKRSE GKHLSANEKK YKDLPKFKTL YKQIGCGKKK DLFYTIKCDT | |
| GW2011_GWA2_33_10] | EEEANKSRNE GKESHSVEEI INKAQEAINK YFKSNNDCEN INTVPDFINY | |
| | ILTKENYEGV YWSKAAMNTI SDKYFANYHD LQDRLKEAKV FQKADKKSED | |
| | DIKIPEAIEL SGLFGVLDSL ADWQTTLFKS SILSNEDKLK IITDSQTPSE | |
| | ALLKMIFNDI EKNMESFLKE TNDIITLKKY KGNKEGTEKI KQWFDYTLAI | |
| | NRMLKYFLVK ENKIKGNSLD TNISEALKTL IYSDDAEWFK WYDALRNYLT | |
| | QKPQDEAKEN KLKLNFDNPS LAGGWDVNKE CSNFCVILKD KNEKKYLAIM | |
| | KKGENTLFQK EWTEGRGKNL TKKSNPLFEI NNCEILSKME YDFWADVSKM | |
| | IPKCSTQLKA VVNHFKQSDN EFIFPIGYKV TSGEKFREEC KISKQDFELN | |
| | NKVFNKNELS VTAMRYDLSS TQEKQYIKAF QKEYWELLFK QEKRDTKLTN | |
| | NEIFNEWINF CNKKYSELLS WERKYKDALT NWINFCKYFL SKYPKTTLFN | |
| | YSFKESENYN SLDEFYRDVD ICSYKLNINT TINKSILDRL VEEGKLYLFE | |
| | IKNQDSNDGK SIGHKNNLHT IYWNAIFENF DNRPKLNGEA EIFYRKAISK | |
| | DKLGIVKGKK TKNGTEIIKN YRFSKEKFIL HVPITLNFCS NNEYVNDIVN | |
| | TKFYNFSNLH FLGIDRGEKH LAYYSLVNKN GEIVDQGTLN LPFTDKDGNQ | |
| | RSIKKEKYFY NKQEDKWEAK EVDCWNYNDL LDAMASNRDM ARKNWQRIGT | |
| | IKEAKNGYVS LVIRKIADLA VNNERPAFIV LEDLNTGFKR SRQKIDKSVY | |
| | QKFELALAKK LNFLVDKNAK RDEIGSPTKA LQLTPPVNNY GDIENKKQAG | |
| | IMLYTRANYT SQTDPATGWR KTIYLKAGPE ETTYKKDGKI KNKSVKDQII | |
| | ETFTDIGFDG KDYYFEYDKG EFVDEKTGEI KPKKWRLYSG ENGKSLDRFR | |
| | GEREKDKYEW KIDKIDIVKI LDDLFVNFDK NISLLKQLKE GVELTRNNEH | |
| | GTGESLRFAI NLIQQIRNTG NNERDNDFIL SPVRDENGKH FDSREYWDKE | |
| | TKGEKISMPS SGDANGAFNI ARKGIIMNAH ILANSDSKDL SLFVSDEEWD | |
| | LHLNNKTEWK KQLNIFSSRK AMAKRKK | |
| KKR91555_(modified) | MLFFMSTDIT NKPREKGVFD NFTNLYEFSK TLTFGLIPLK WDDNKKMIVE | (SEQ ID |
| hypothetical | DEDFSVLRKY GVIEEDKRIA ESIKIAKFYL NILHRELIGK VLGSLKFEKK | NO: 117) |
| protein | NLENYDRLLG EIEKNNKNEN ISEDKKKEIR KNFKKELSIA QDILLKKVGE | |
| UU43_C0004G0003 | VFESNGSGIL SSKNCLDELT KRFTRQEVDK LRRENKDIGV EYPDVAYREK | |
| [Parcubacteria | DGKEETKSFF AMDVGYLDDF HKNRKQLYSV KGKKNSLGRR ILDNFEIFCK | |
| (Falkowbacteria) | NKKLYEKYKN LDIDFSEIER NFNLTLEKVF DFDNYNERLT QEGLDEYAKI | |
| bacterium | LGGESNKQER TANIHGLNQI INLYIQKKQS EQKAEQKETG KKKIKFNKKD | |
| GW2011_GWA2_41_14] | YPTFTCLQKQ ILSQVFRKEI IIESDRDLIR ELKFFVEESK EKVDKARGII | |
| | EFLLNHEEND IDLAMVYLPK SKINSFVYKV FKEPQDFLSV FQDGASNLDF | |
| | VSFDKIKTHL ENNKLTYKIF FKTLIKENHD FESFLILLQQ EIDLLIDGGE | |
| | TVTLGGKKES ITSLDEKKNR LKEKLGWFEG KVRENEKMKD EEEGEFCSTV | |
| | LAYSQAVLNI TKRAEIFWLN EKQDAKVGED NKDMIFYKKF DEFADDGFAP | |
| | FFYFDKFGNY LKRRSRNTTK EIKLHFGNDD LLEGWDMNKE PEYWSFILRD | |
| | RNQYYLGIGK KDGEIFHKKL GNSVEAVKEA YELENEADFY EKIDYKQLNI | |
| | DRFEGIAFPK KTKTEEAFRQ VCKKRADEFL GGDTYEFKIL LAIKKEYDDF | |
| | KARRQKEKDW DSKFSKEKMS KLIEYYITCL GKRDDWKRFN LNFRQPKEYE | |
| | DRSDFVRHIQ RQAYWIDPRK VSKDYVDKKV AEGEMFLFKV HNKDFYDFER | |
| | KSEDKKNHTA NLFTQYLLEL FSCENIKNIK SKDLIESIFE LDGKAEIRFR | |
| | PKTDDVKLKI YQKKGKDVTY ADKRDGNKEK EVIQHRRFAK DALTLHLKIR | |
| | LNFGKHVNLF DFNKLVNTEL FAKVPVKILG MDRGENNLIY YCFLDEHGEI | |
| | ENGKCGSLNR VGEQIITLED DKKVKEPVDY FQLLVDREGQ RDWEQKNWQK | |
| | MTRIKDLKKA YLGNVVSWIS KEMLSGIKEG VVTIGVLEDL NSNFKRTRFF | |
| | RERQVYQGFE KALVNKLGYL VDKKYDNYRN VYQFAPIVDS VEEMEKNKQI | |
| | GTLVYVPASY TSKICPHPKC GWRERLYMKN SASKEKIVGL LKSDGIKISY | |
| | DQKNDRFYFE YQWEQEHKSD GKKKKYSGVD KVFSNVSRMR WDVEQKKSID | |
| | FVDGTDGSIT NKLKSLLKGK GIELDNINQQ IVNQQKELGV EFFQSIIFYF | |
| | NLIMQIRNYD KEKSGSEADY IQCPSCLFDS RKPEMNGKLS AITNGDANGA | |
| | YNIARKGFMQ LCRIRENPQE PMKLITNREW DEAVREWDIY SAAQKIPVLS | |
| | EEN | |
| KDN25524_(modified) | MLFQDFTHLY PLSKTVRFEL KPIDRTLEHI HAKNFLSQDE TMADMHQKVK | (SEQ ID |
| hypothetical | VILDDYHRDF IADMMGEVKL TKLAEFYDVY LKFRKNPKDD ELQKQLKDLQ | NO: 118) |
| protein | AVLRKEIVKP IGNGGKYKAG YDRLFGAKLF KDGKELGDLA KFVIAQEGES | |
| MBO 03467 | SPKLAHLAHF EKFSTYFTGF HDNRKNMYSD EDKHTAIAYR LIHENLPRFI | |
| [Moraxella bovoculi | DNLQILTTIK QKHSALYDQI INELTASGLD VSLASHLDGY HKLLTQEGIT | |
| 237] | AYNTLLGGIS GEAGSPKIQG INELINSHHN QHCHKSERIA KLRPLHKQIL | |
| > WP_052585281.1 | SDGMSVSFLP SKFADDSEMC QAVNEFYRHY ADVFAKVQSL FDGFDDHQKD | |
| type V CRISPR- | GIYVEHKNLN ELSKQAFGDF ALLGRVLDGY YVDVVNPEFN ERFAKAKTDN | |
| associated protein | AKAKLTKEKD KFIKGVHSLA SLEQAIEHYT ARHDDESVQA GKLGQYFKHG | |
| Cpf1 [Moraxella | LAGVDNPIQK IHNNHSTIKG FLERERPAGE RALPKIKSGK NPEMTQLRQL | |
| bovoculi] | KELLDNALNV AHFAKLLTTK TTLDNQDGNF YGEFGVLYDE LAKIPTLYNK | |
| | VRDYLSQKPF STEKYKLNFG NPTLLNGWDL NKEKDNFGVI LQKDGCYYLA | |
| | LLDKAHKKVF DNAPNTGKSI YQKMIYKYLE VRKQFPKVFF SKEAIAINYH | |
| | PSKELVEIKD KGRQRSDDER LKLYRFILEC LKIHPKYDKK FEGAIGDIQL | |
| | FKKDKKGREV PISEKDLFDK INGIFSSKPK LEMEDFFIGE FKRYNPSQDL | |
| | VDQYNIYKKI DSNDNRKKEN FYNNHPKFKK DLVRYYYESM CKHEEWEESF | |
| | EFSKKLQDIG CYVDVNELFT EIETRRLNYK ISFCNINADY IDELVEQGQL | |
| | YLFQIYNKDF SPKAHGKPNL HTLYFKALFS EDNLADPIYK LNGEAQIFYR | |
| | KASLDMNETT IHRAGEVLEN KNPDNPKKRQ FVYDIIKDKR YTQDKFMLHV | |
| | PITMNFGVQG MTIKEFNKKV NQSIQQYDEV NVIGIDRGER HLLYLTVINS | |
| | KGEILEQCSL NDITTASANG TQMTTPYHKI LDKREIERLN ARVGWGEIET | |
| | IKELKSGYLS HVVHQISQLM LKYNAIVVLE DLNFGFKRGR FKVEKQIYQN | |
| | FENALIKKLN HLVLKDKADD EIGSYKNALQ LTNNFTDLKS IGKQTGFLFY | |
| | VPAWNTSKID PETGFVDLLK PRYENIAQSQ AFFGKFDKIC YNADKDYFEF | |
| | HIDYAKFTDK AKNSRQIWTI CSHGDKRYVY DKTANQNKGA AKGINVNDEL | |
| | KSLFARHHIN EKQPNLVMDI CQNNDKEFHK SLMYLLKTLL ALRYSNASSD | |
| | EDFILSPVAN DEGVFFNSAL ADDTQPQNAD ANGAYHIALK GLWLLNELKN | |
| | SDDLNKVKLA IDNQTWLNFA QNR | |
| KKT48220_(modified) | MENIFDQFIG KYSLSKTLRF ELKPVGKTED FLKINKVFEK DQTIDDSYNQ | (SEQ ID |
| hypothetical | AKFYFDSLHQ KFIDAALASD KTSELSFQNF ADVLEKQNKI ILDKKREMGA | NO: 119) |
| protein | LRKRDKNAVG IDRLQKEIND AEDIIQKEKE KIYKDVRTLF DNEAESWKTY | |
| UW39 C0001G0044 | YQEREVDGKK ITFSKADLKQ KGADFLTAAG ILKVLKYEFP EEKEKEFQAK | |
| [Parcubacteria | NQPSLFVEEK ENPGQKRYIF DSFDKFAGYL TKFQQTKKNL YAADGTSTAV | |
| bacterium | ATRIADNFII FHQNTKVFRD KYKNNHTDLG FDEENIFEIE RYKNCLLQRE | |
| GW2011_GWC2_44_17] | IEHIKNENSY NKIIGRINKK IKEYRDQKAK DTKLTKSDFP FFKNLDKQIL | |
| | GEVEKEKQLI EKTREKTEED VLIERFKEFI ENNEERFTAA KKLMNAFCNG | |
| | EFESEYEGIY LKNKAINTIS RRWFVSDRDF ELKLPQQKSK NKSEKNEPKV | |
| | KKFISIAEIK NAVEELDGDI FKAVFYDKKI IAQGGSKLEQ FLVIWKYEFE | |
| | YLFRDIEREN GEKLLGYDSC LKIAKQLGIF PQEKEAREKA TAVIKNYADA | |
| | GLGIFQMMKY FSLDDKDRKN TPGQLSTNFY AEYDGYYKDF EFIKYYNEFR | |
| | NFITKKPFDE DKIKLNFENG ALLKGWDENK EYDFMGVILK KEGRLYLGIM | |
| | HKNHRKLFQS MGNAKGDNAN RYQKMIYKQI ADASKDVPRL LLTSKKAMEK | |
| | FKPSQEILRI KKEKTFKRES KNFSLRDLHA LIEYYRNCIP QYSNWSFYDF | |
| | QFQDTGKYQN IKEFTDDVQK YGYKISFRDI DDEYINQALN EGKMYLFEVV | |
| | NKDIYNTKNG SKNLHTLYFE HILSAENLND PVFKLSGMAE IFQRQPSVNE | |
| | REKITTQKNQ CILDKGDRAY KYRRYTEKKI MFHMSLVLNT GKGEIKQVQF | |
| | NKIINQRISS SDNEMRVNVI GIDRGEKNLL YYSVVKQNGE IIEQASLNEI | |
| | NGVNYRDKLI EREKERLKNR QSWKPVVKIK DLKKGYISHV IHKICQLIEK | |
| | YSAIVVLEDL NMRFKQIRGG IERSVYQQFE KALIDKLGYL VFKDNRDLRA | |
| | PGGVLNGYQL SAPFVSFEKM RKQTGILFYT QAEYTSKTDP ITGFRKNVYI | |
| | SNSASLDKIK EAVKKFDAIG WDGKEQSYFF KYNPYNLADE KYKNSTVSKE | |
| | WAIFASAPRI RRQKGEDGYW KYDRVKVNEE FEKLLKVWNF VNPKATDIKQ | |
| | EIIKKEKAGD LQGEKELDGR LRNFWHSFIY LFNLVLELRN SFSLQIKIKA | |
| | GEVIAVDEGV DFIASPVKPF FTTPNPYIPS NLCWLAVENA DANGAYNIAR | |
| | KGVMILKKIR EHAKKDPEFK KLPNLFISNA EWDEAARDWG KYAGTTALNL | |
| | DH | |
| WP_031492824_(modified) | MSSLTKFTNK YSKQLTIKNE LIPVGKTLEN IKENGLIDGD EQLNENYQKA | (SEQ ID |
| hypothetical protein | KIIVDDFLRD FINKALNNTQ IGNWRELADA LNKEDEDNIE KLQDKIRGII | NO: 120) |
| [Succinivibrio | VSKFETFDLF SSYSIKKDEK IIDDDNDVEE EELDLGKKTS SFKYIFKKNL | |
| dextrinosolvens] | FKLVLPSYLK TTNQDKLKII SSFDNFSTYF RGFFENRKNI FTKKPISTSI | |
| | AYRIVHDNFP KFLDNIRCFN VWQTECPQLI VKADNYLKSK NVIAKDKSLA | |
| | NYFTVGAYDY FLSQNGIDFY NNIIGGLPAF AGHEKIQGLN EFINQECQKD | |
| | SELKSKLKNR HAFKMAVLFK QILSDREKSF VIDEFESDAQ VIDAVKNFYA | |
| | EQCKDNNVIF NLLNLIKNIA FLSDDELDGI FIEGKYLSSV SQKLYSDWSK | |
| | LRNDIEDSAN SKQGNKELAK KIKTNKGDVE KAISKYEFSL SELNSIVHDN | |
| | TKFSDLLSCT LHKVASEKLV KVNEGDWPKH LKNNEEKQKI KEPLDALLEI | |
| | YNTLLIFNCK SFNKNGNFYV DYDRCINELS SVVYLYNKTR NYCTKKPYNT | |
| | DKFKLNFNSP QLGEGFSKSK ENDCLTLLFK KDDNYYVGII RKGAKINFDD | |
| | TQAIADNTDN CIFKMNYFLL KDAKKFIPKC SIQLKEVKAH FKKSEDDYIL | |
| | SDKEKFASPL VIKKSTFLLA TAHVKGKKGN IKKFQKEYSK ENPTEYRNSL | |
| | NEWIAFCKEF LKTYKAATIF DITTLKKAEE YADIVEFYKD VDNLCYKLEF | |
| | CPIKTSFIEN LIDNGDLYLF RINNKDFSSK STGTKNLHTL YLQAIFDERN | |
| | LNNPTIMLNG GAELFYRKES IEQKNRITHK AGSILVNKVC KDGTSLDDKI | |
| | RNEIYQYENK FIDTLSDEAK KVLPNVIKKE ATHDITKDKR FTSDKFFFHC | |
| | PLTINYKEGD TKQFNNEVLS FLRGNPDINI IGIDRGERNL IYVTVINQKG | |
| | EILDSVSFNT VTNKSSKIEQ TVDYEEKLAV REKERIEAKR SWDSISKIAT | |
| | LKEGYLSAIV HEICLLMIKH NAIVVLENLN AGFKRIRGGL SEKSVYQKFE | |
| | KMLINKLNYF VSKKESDWNK PSGLINGLQL SDQFESFEKL GIQSGFIFYV | |
| | PAAYTSKIDP TTGFANVLNL SKVRNVDAIK SFFSNFNEIS YSKKEALFKF | |
| | SFDLDSLSKK GFSSFVKFSK SKWNVYTFGE RIIKPKNKQG YREDKRINLT | |
| | FEMKKLLNEY KVSFDLENNL IPNLTSANLK DTFWKELFFI FKTTLQLRNS | |
| | VTNGKEDVLI SPVKNAKGEF FVSGTHNKTL PQDCDANGAY HIALKGLMIL | |
| | ERNNLVREEK DTKKIMAISN VDWFEYVQKR RGVL | |
| KKT50231_(modified) | MKPVGKTEDF LKINKVFEKD QTIDDSYNQA KFYFDSLHQK FIDAALASDK | (SEQ ID |
| hypothetical | TSELSFQNFA DVLEKQNKII LDKKREMGAL RKRDKNAVGI DRLQKEINDA | NO: 121) |
| protein | EDIIQKEKEK IYKDVRTLFD NEAESWKTYY QEREVDGKKI TFSKADLKQK | |
| UW40 C0007G0006 | GADFLTAAGI LKVLKYEFPE EKEKEFQAKN QPSLFVEEKE NPGQKRYIFD | |
| [Parcubacteria | SFDKFAGYLT KFQQTKKNLY AADGTSTAVA TRIADNFIIF HQNTKVFRDK | |
| bacterium | YKNNHTDLGF DEENIFEIER YKNCLLQREI EHIKNENSYN KIIGRINKKI | |
| GW2011_GWF2_44_17] | KEYRDQKAKD TKLTKSDFPF FKNLDKQILG EVEKEKQLIE KTREKTEEDV | |
| | LIERFKEFIE NNEERFTAAK KLMNAFCNGE FESEYEGIYL KNKAINTISR | |
| | RWFVSDRDFE LKLPQQKSKN KSEKNEPKVK KFISIAEIKN AVEELDGDIF | |
| | KAVFYDKKII AQGGSKLEQF LVIWKYEFEY LFRDIERENG EKLLGYDSCL | |
| | KIAKQLGIFP QEKEAREKAT AVIKNYADAG LGIFQMMKYF SLDDKDRKNT | |
| | PGQLSTNFYA EYDGYYKDFE FIKYYNEFRN FITKKPFDED KIKLNFENGA | |
| | LLKGWDENKE YDFMGVILKK EGRLYLGIMH KNHRKLFQSM GNAKGDNANR | |
| | YQKMIYKQIA DASKDVPRLL LTSKKAMEKF KPSQEILRIK KEKTFKRESK | |
| | NFSLRDLHAL IEYYRNCIPQ YSNWSFYDFQ FQDTGKYQNI KEFTDDVQKY | |
| | GYKISFRDID DEYINQALNE GKMYLFEVVN KDIYNTKNGS KNLHTLYFEH | |
| | ILSAENLNDP VFKLSGMAEI FQRQPSVNER EKITTQKNQC ILDKGDRAYK | |
| | YRRYTEKKIM FHMSLVLNTG KGEIKQVQFN KIINQRISSS DNEMRVNVIG | |
| | IDRGEKNLLY YSVVKQNGEI IEQASLNEIN GVNYRDKLIE REKERLKNRQ | |
| | SWKPVVKIKD LKKGYISHVI HKICQLIEKY SAIVVLEDLN MRFKQIRGGI | |
| | ERSVYQQFEK ALIDKLGYLV FKDNRDLRAP GGVLNGYQLS APFVSFEKMR | |
| | KQTGILFYTQ AEYTSKTDPI TGFRKNVYIS NSASLDKIKE AVKKFDAIGW | |
| | DGKEQSYFFK YNPYNLADEK YKNSTVSKEW AIFASAPRIR RQKGEDGYWK | |
| | YDRVKVNEEF EKLLKVWNFV NPKATDIKQE IIKKEKAGDL QGEKELDGRL | |
| | RNFWHSFIYL FNLVLELRNS FSLQIKIKAG EVIAVDEGVD FIASPVKPFF | |
| | TTPNPYIPSN LCWLAVENAD ANGAYNIARK GVMILKKIRE HAKKDPEFKK | |
| | LPNLFISNAE WDEAARDWGK YAGTTALNLD H | |
| WP_004356401_(modified) | MKVMENYQEF TNLFQLNKTL RFELKPIGKT CELLEEGKIF ASGSFLEKDK | (SEQ ID |
| hypothetical protein | VRADNVSYVK KEIDKKHKIF IEETLSSFSI SNDLLKQYFD CYNELKAFKK | NO: 122) |
| [Prevotella disiens] | DCKSDEEEVK KTALRNKCTS IQRAMREAIS QAFLKSPQKK LLAIKNLIEN | |
| | VFKADENVQH FSEFTSYFSG FETNRENFYS DEEKSTSIAY RLVHDNLPIF | |
| | IKNIYIFEKL KEQFDAKTLS EIFENYKLYV AGSSLDEVFS LEYFNNTLTQ | |
| | KGIDNYNAVI GKIVKEDKQE IQGLNEHINL YNQKHKDRRL PFFISLKKQI | |
| | LSDREALSWL PDMFKNDSEV IKALKGFYIE DGFENNVLTP LATLLSSLDK | |
| | YNLNGIFIRN NEALSSLSQN VYRNFSIDEA IDANAELQTF NNYELIANAL | |
| | RAKIKKETKQ GRKSFEKYEE YIDKKVKAID SLSIQEINEL VENYVSEFNS | |
| | NSGNMPRKVE DYFSLMRKGD FGSNDLIENI KTKLSAAEKL LGTKYQETAK | |
| | DIFKKDENSK LIKELLDATK QFQHFIKPLL GTGEEADRDL VFYGDFLPLY | |
| | EKFEELTLLY NKVRNRLTQK PYSKDKIRLC FNKPKLMTGW VDSKTEKSDN | |
| | GTQYGGYLFR KKNEIGEYDY FLGISSKAQL FRKNEAVIGD YERLDYYQPK | |
| | ANTIYGSAYE GENSYKEDKK RINKVIIAYI EQIKQTNIKK SIIESISKYP | |
| | NISDDDKVTP SSLLEKIKKV SIDSYNGILS FKSFQSVNKE VIDNLLKTIS | |
| | PLKNKAEFLD LINKDYQIFT EVQAVIDEIC KQKTFIYFPI SNVELEKEMG | |
| | DKDKPLCLFQ ISNKDLSFAK TFSANLRKKR GAENLHTMLF KALMEGNQDN | |
| | LDLGSGAIFY RAKSLDGNKP THPANEAIKC RNVANKDKVS LFTYDIYKNR | |
| | RYMENKFLFH LSIVQNYKAA NDSAQLNSSA TEYIRKADDL HIIGIDRGER | |
| | NLLYYSVIDM KGNIVEQDSL NIIRNNDLET DYHDLLDKRE KERKANRQNW | |
| | EAVEGIKDLK KGYLSQAVHQ IAQLMLKYNA IIALEDLGQM FVTRGQKIEK | |
| | AVYQQFEKSL VDKLSYLVDK KRPYNELGGI LKAYQLASSI TKNNSDKQNG | |
| | FLFYVPAWNT SKIDPVTGFT DLLRPKAMTI KEAQDFFGAF DNISYNDKGY | |
| | FEFETNYDKF KIRMKSAQTR WTICTFGNRI KRKKDKNYWN YEEVELTEEF | |
| | KKLFKDSNID YENCNLKEEI QNKDNRKFFD DLIKLLQLTL QMRNSDDKGN | |
| | DYIISPVANA EGQFFDSRNG DKKLPLDADA NGAYNIARKG LWNIRQIKQT | |
| | KNDKKLNLSI SSTEWLDFVR EKPYLK | |
| CCB70584_(modified) | MTNKFTNQYS LSKTLRFELI PQGKTLEFIQ EKGLLSQDKQ RAESYQEMKK | (SEQ ID |
| Protein of | TIDKFHKYFI DLALSNAKLT HLETYLELYN KSAETKKEQK FKDDLKKVQD | NO: 123) |
| unknown function | NLRKEIVKSF SDGDAKSIFA ILDKKELITV ELEKWFENNE QKDIYFDEKF | |
| [Flavobacterium | KTFTTYFTGF HQNRKNMYSV EPNSTAIAYR LIHENLPKFL ENAKAFEKIK | |
| branchiophilum FL-15] | QVESLQVNFR ELMGEFGDEG LIFVNELEEM FQINYYNDVL SQNGITIYNS | |
| | IISGFTKNDI KYKGLNEYIN NYNQTKDKKD RLPKLKQLYK QILSDRISLS | |
| | FLPDAFTDGK QVLKAIFDFY KINLLSYTIE GQEESQNLLL LIRQTIENLS | |
| | SFDTQKIYLK NDTHLTTISQ QVFGDFSVFS TALNYWYETK VNPKFETEYS | |
| | KANEKKREIL DKAKAVFTKQ DYFSIAFLQE VLSEYILTLD HTSDIVKKHS | |
| | SNCIADYFKN HFVAKKENET DKTFDFIANI TAKYQCIQGI LENADQYEDE | |
| | LKQDQKLIDN LKFFLDAILE LLHFIKPLHL KSESITEKDT AFYDVFENYY | |
| | EALSLLTPLY NMVRNYVTQK PYSTEKIKLN FENAQLLNGW DANKEGDYLT | |
| | TILKKDGNYF LAIMDKKHNK AFQKFPEGKE NYEKMVYKLL PGVNKMLPKV | |
| | FFSNKNIAYF NPSKELLENY KKETHKKGDT FNLEHCHTLI DFFKDSLNKH | |
| | EDWKYFDFQF SETKSYQDLS GFYREVEHQG YKINFKNIDS EYIDGLVNEG | |
| | KLFLFQIYSK DFSPFSKGKP NMHTLYWKAL FEEQNLQNVI YKLNGQAEIF | |
| | FRKASIKPKN IILHKKKIKI AKKHFIDKKT KTSEIVPVQT IKNLNMYYQG | |
| | KISEKELTQD DLRYIDNFSI FNEKNKTIDI IKDKRFTVDK FQFHVPITMN | |
| | FKATGGSYIN QTVLEYLQNN PEVKIIGLDR GERHLVYLTL IDQQGNILKQ | |
| | ESLNTITDSK ISTPYHKLLD NKENERDLAR KNWGTVENIK ELKEGYISQV | |
| | VHKIATLMLE ENAIVVMEDL NFGFKRGRFK VEKQIYQKLE KMLIDKLNYL | |
| | VLKDKQPQEL GGLYNALQLT NKFESFQKMG KQSGFLFYVP AWNTSKIDPT | |
| | TGFVNYFYTK YENVDKAKAF FEKFEAIRFN AEKKYFEFEV KKYSDFNPKA | |
| | EGTQQAWTIC TYGERIETKR QKDQNNKFVS TPINLTEKIE DFLGKNQIVY | |
| | GDGNCIKSQI ASKDDKAFFE TLLYWFKMTL QMRNSETRTD IDYLISPVMN | |
| | DNGTFYNSRD YEKLENPTLP KDADANGAYH IAKKGLMLLN KIDQADLTKK | |
| | VDLSISNRDW LQFVQKNK | |
| WP_005398606_(modified) | MFEKLSNIVS ISKTIRFKLI PVGKTLENIE KLGKLEKDFE RSDFYPILKN | (SEQ ID |
| hypothetical protein | ISDDYYRQYI KEKLSDLNLD WQKLYDAHEL LDSSKKESQK NLEMIQAQYR | NO: 124) |
| [Helcococcus | KVLFNILSGE LDKSGEKNSK DLIKNNKALY GKLFKKQFIL EVLPDFVNNN | |
| kunzii] | DSYSEEDLEG LNLYSKFTTR LKNFWETRKN VFTDKDIVTA IPFRAVNENF | |
| | GFYYDNIKIF NKNIEYLENK IPNLENELKE ADILDDNRSV KDYFTPNGFN | |
| | YVITQDGIDV YQAIRGGFTK ENGEKVQGIN EILNLTQQQL RRKPETKNVK | |
| | LGVLTKLRKQ ILEYSESTSF LIDQIEDDND LVDRINKFNV SFFESTEVSP | |
| | SLFEQIERLY NALKSIKKEE VYIDARNTQK FSQMLFGQWD VIRRGYTVKI | |
| | TEGSKEEKKK YKEYLELDET SKAKRYLNIR EIEELVNLVE GFEEVDVFSV | |
| | LLEKFKMNNI ERSEFEAPIY GSPIKLEAIK EYLEKHLEEY HKWKLLLIGN | |
| | DDLDTDETFY PLLNEVISDY YIIPLYNLTR NYLTRKHSDK DKIKVNFDFP | |
| | TLADGWSESK ISDNRSIILR KGGYYYLGIL IDNKLLINKK NKSKKIYEIL | |
| | IYNQIPEFSK SIPNYPFTKK VKEHFKNNVS DFQLIDGYVS PLIITKEIYD | |
| | IKKEKKYKKD FYKDNNTNKN YLYTIYKWIE FCKQFLYKYK GPNKESYKEM | |
| | YDFSTLKDTS LYVNLNDFYA DVNSCAYRVL FNKIDENTID NAVEDGKLLL | |
| | FQIYNKDFSP ESKGKKNLHT LYWLSMFSEE NLRTRKLKLN GQAEIFYRKK | |
| | LEKKPIIHKE GSILLNKIDK EGNTIPENIY HECYRYLNKK IGREDLSDEA | |
| | IALFNKDVLK YKEARFDIIK DRRYSESQFF FHVPITFNWD IKTNKNVNQI | |
| | VQGMIKDGEI KHIIGIDRGE RHLLYYSVID LEGNIVEQGS LNTLEQNRFD | |
| | NSTVKVDYQN KLRTREEDRD RARKNWTNIN KIKELKDGYL SHVVHKLSRL | |
| | IIKYEAIVIM ENLNQGFKRG RFKVERQVYQ KFELALMNKL SALSFKEKYD | |
| | ERKNLEPSGI LNPIQACYPV DAYQELQGQN GIVFYLPAAY TSVIDPVTGF | |
| | TNLFRLKSIN SSKYEEFIKK FKNIYFDNEE EDFKFIFNYK DFAKANLVIL | |
| | NNIKSKDWKI STRGERISYN SKKKEYFYVQ PTEFLINKLK ELNIDYENID | |
| | IIPLIDNLEE KAKRKILKAL FDTFKYSVQL RNYDFENDYI ISPTADDNGN | |
| | YYNSNEIDID KTNLPNNGDA NGAFNIARKG LLLKDRIVNS NESKVDLKIK | |
| | NEDWINFIIS | |
| WP_021736722_(modified) | MTQFEGFTNL YQVSKTLRFE LIPQGKTLKH IQEQGFIEED KARNDHYKEL | (SEQ ID |
| CRISPR- | KPIIDRIYKT YADQCLQLVQ LDWENLSAAI DSYRKEKTEE TRNALIEEQA | NO: 125) |
| associated protein | TYRNAIHDYF IGRTDNLTDA INKRHAEIYK GLFKAELFNG KVLKQLGTVT | |
| Cpf1, subtype | TTEHENALLR SFDKFTTYFS GFYENRKNVF SAEDISTAIP HRIVQDNFPK | |
| PREFRAN | FKENCHIFTR LITAVPSLRE HFENVKKAIG IFVSTSIEEV FSFPFYNQLL | |
| [Acidaminococcus | TQTQIDLYNQ LLGGISREAG TEKIKGLNEV LNLAIQKNDE TAHIIASLPH | |
| sp. BV3L6] | RFIPLFKQIL SDRNTLSFIL EEFKSDEEVI QSFCKYKTLL RNENVLETAE | |
| | ALFNELNSID LTHIFISHKK LETISSALCD HWDTLRNALY ERRISELTGK | |
| | ITKSAKEKVQ RSLKHEDINL QEIISAAGKE LSEAFKQKTS EILSHAHAAL | |
| | DQPLPTTLKK QEEKEILKSQ LDSLLGLYHL LDWFAVDESN EVDPEFSARL | |
| | TGIKLEMEPS LSFYNKARNY ATKKPYSVEK FKLNFQMPTL ASGWDVNKEK | |
| | NNGAILFVKN GLYYLGIMPK QKGRYKALSF EPTEKTSEGF DKMYYDYFPD | |
| | AAKMIPKCST QLKAVTAHFQ THTTPILLSN NFIEPLEITK EIYDLNNPEK | |
| | EPKKFQTAYA KKTGDQKGYR EALCKWIDFT RDFLSKYTKT TSIDLSSLRP | |
| | SSQYKDLGEY YAELNPLLYH ISFQRIAEKE IMDAVETGKL YLFQIYNKDF | |
| | AKGHHGKPNL HTLYWTGLFS PENLAKTSIK LNGQAELFYR PKSRMKRMAH | |
| | RLGEKMLNKK LKDQKTPIPD TLYQELYDYV NHRLSHDLSD EARALLPNVI | |
| | TKEVSHEIIK DRRFTSDKFF FHVPITLNYQ AANSPSKFNQ RVNAYLKEHP | |
| | ETPIIGIDRG ERNLIYITVI DSTGKILEQR SLNTIQQFDY QKKLDNREKE | |
| | RVAARQAWSV VGTIKDLKQG YLSQVIHEIV DLMIHYQAVV VLENLNFGFK | |
| | SKRTGIAEKA VYQQFEKMLI DKLNCLVLKD YPAEKVGGVL NPYQLTDQFT | |
| | SFAKMGTQSG FLFYVPAPYT SKIDPLTGFV DPFVWKTIKN HESRKHFLEG | |
| | FDFLHYDVKT GDFILHFKMN RNLSFQRGLP GFMPAWDIVF EKNETQFDAK | |
| | GTPFIAGKRI VPVIENHRFT GRYRDLYPAN ELIALLEEKG IVFRDGSNIL | |
| | PKLLENDDSH AIDTMVALIR SVLQMRNSNA ATGEDYINSP VRDLNGVCFD | |
| | SRFQNPEWPM DADANGAYHI ALKGQLLLNH LKESKDLKLQ NGISNQDWLA | |
| | YIQELRN | |
| WP_004339290_(modified) | MSIYQEFVNK YSLSKTLRFE LIPQGKTLEN IKARGLILDD EKRAKDYKKA | (SEQ ID |
| odified) | KQIIDKYHQF FIEEILSSVC ISEDLLQNYS DVYFKLKKSD DDNLQKDFKS | NO: 126) |
| hypothetical protein | AKDTIKKQIS KYINDSEKFK NLFNQNLIDA KKGQESDLIL WLKQSKDNGI | |
| [Francisella | ELFKANSDIT DIDEALEIIK SFKGWTTYFK GFHENRKNVY SSNDIPTSII | |
| tularensis] | YRIVDDNLPK FLENKAKYES LKDKAPEAIN YEQIKKDLAE ELTFDIDYKT | |
| | SEVNQRVFSL DEVFEIANFN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLFDD LKAQKLDLSK IYFKNDKSLT | |
| | DLSQQVFDDY SVIGTAVLEY ITQQVAPKNL DNPSKKEQDL IAKKTEKAKY | |
| | LSLETIKLAL EEFNKHRDID KQCRFEEILS NFAAIPMIFD EIAQNKDNLA | |
| | QISIKYQNQG KKDLLQASAE EDVKAIKDLL DQTNNLLHRL KIFHISQSED | |
| | KANILDKDEH FYLVFEECYF ELANIVPLYN KIRNYITQKP YSDEKFKLNF | |
| | ENSTLASGWD KNKESANTAI LFIKDDKYYL GIMDKKHNKI FSDKAIEENK | |
| | GEGYKKIVYK QIADASKDIQ NLMIIDGKTV CKKGRKDRNG VNRQLLSLKR | |
| | KHLPENIYRI KETKSYLKNE ARFSRKDLYD FIDYYKDRLD YYDFEFELKP | |
| | SNEYSDFNDF TNHIGSQGYK LTFENISQDY INSLVNEGKL YLFQTYSKDF | |
| | SAYSKGRPNL HTLYWKALFD ERNLQDVVYK LNGEAELFYR KQSIPKKITH | |
| | PAKETIANKN KDNPKKESVF EYDLIKDKRF TEDKFFFHCP ITINFKSSGA | |
| | NKFNDEINLL LKEKANDVHI LSIDRGERHL AYYTLVDGKG NIIKQDNFNI | |
| | IGNDRMKTNY HDKLAAIEKD RDSARKDWKK INNIKEMKEG YLSQVVHEIA | |
| | KLVIEYNAIV VFEDLNFGFK RGRFKVEKQV YQKLEKMLIE KLNYLVFKDN | |
| | EFDKTGGVLR AYQLTAPFET FKKMGKQTGI IYYVPAGFTS KICPVTGFVN | |
| | QLYPKYESVS KSQEFFSKFD KICYNLDKGY FEFSFDYKNF GDKAAKGKWT | |
| | IASFGSRLIN FRNSDKNHNW DTREVYPTKE LEKLLKDYSI EYGHGECIKA | |
| | AICGESDKKF FAKLTSVLNT ILQMRNSKTG TELDYLISPV ADVNGNFFDS | |
| | RQAPKNMPQD ADANGAYHIG LKGLMLLDRI KNNQEGKKLN LVIKNEEYFE | |
| | FVQNRNN | |
| WP_022501477 | MNKAADNYTG GNYDEFIALS KVQKTLRNEL KPTPFTAEHI KQRGIISEDE | (SEQ ID |
| type V CRISPR- | YRAQQSLELK KIADEYYRNY ITHKLNDINN LDFYNLFDAI EEKYKKNDKD | NO: 127) |
| associated protein | NRDKLDLVEK SKRGEIAKML SADDNFKSMF EAKLITKLLP DYVERNYTGE | |
| Cpf1 [Eubacterium | DKEKALETLA LFKGFTTYFK GYFKTRKNMF SGEGGASSIC HRIVNVNASI | |
| sp. CAG:76] | FYDNLKTFMR IQEKAGDEIA LIEEELTEKL DGWRLEHIFS RDYYNEVLAQ | |
| | KGIDYYNQIC GDINKHMNLY CQQNKFKANI FKMMKIQKQI MGISEKAFEI | |
| | PPMYQNDEEV YASFNEFISR LEEVKLTDRL INILQNINIY NTAKIYINAR | |
| | YYTNVSSYVY GGWGVIDSAI ERYLYNTIAG KGQSKVKKIE NAKKDNKFMS | |
| | VKELDSIVAE YEPDYFNAPY IDDDDNAVKA FGGQGVLGYF NKMSELLADV | |
| | SLYTIDYNSD DSLIENKESA LRIKKQLDDI MSLYHWLQTF IIDEVVEKDN | |
| | AFYAELEDIC CELENVVTLY DRIRNYVTKK PYSTQKFKLN FASPTLAAGW | |
| | SRSKEFDNNA IILLRNNKYY IAIFNVNNKP DKQIIKGSEE QRLSTDYKKM | |
| | VYNLLPGPNK MLPKVFIKSD TGKRDYNPSS YILEGYEKNR HIKSSGNFDI | |
| | NYCHDLIDYY KACINKHPEW KNYGFKFKET NQYNDIGQFY KDVEKQGYSI | |
| | SWAYISEEDI NKLDEEGKIY LFEIYNKDLS AHSTGRDNLH TMYLKNIFSE | |
| | DNLKNICIEL NGEAELFYRK SSMKSNITHK KDTILVNKTY INETGVRVSL | |
| | SDEDYMKVYN YYNNNYVIDT ENDKNLIDII EKIGHRKSKI DIVKDKRYTE | |
| | DKYFLYLPIT INYGIEDENV NSKIIEYIAK QDNMNVIGID RGERNLIYIS | |
| | VIDNKGNIIE QKSFNLVNNY DYKNKLKNME KTRDNARKNW QEIGKIKDVK | |
| | SGYLSGVISK IARMVIDYNA IIVMEDLNKG FKRGRFKVER QVYQKFENML | |
| | ISKLNYLVFK ERKADENGGI LRGYQLTYIP KSIKNVGKQC GCIFYVPAAY | |
| | TSKIDPATGF INIFDFKKYS GSGINAKVKD KKEFLMSMNS IRYINECSEE | |
| | YEKIGHRELF AFSFDYNNFK TYNVSSPVNE WTAYTYGERI KKLYKDGRWL | |
| | RSEVLNLTEN LIKLMEQYNI EYKDGHDIRE DISHMDETRN ADFICSLFEE | |
| | LKYTVQLRNS KSEAEDENYD RLVSPILNSS NGFYDSSDYM ENENNTTHTM | |
| | PKDADANGAY CIALKGLYEI NKIKQNWSDD KKFKENELYI NVTEWLDYIQ | |
| | NRRFE | |
| WP_014550095 | MSIYQEFVNK YSLSKTLRFE LIPQGKTLEN IKARGLILDD EKRAKDYKKA | (SEQ ID |
| type V CRISPR- | KQIIDKYHQF FIEEILSSVC ISEDLLQNYS DVYFKLKKSD DDNLQKDFKS | NO: 128) |
| associated protein | AKDTIKKQIS EYIKDSEKFK NLFNQNLIDA KKGQESDLIL WLKQSKDNGI | |
| Cpf1 [Francisella | ELFKANSDIT DIDEALEIIK SFKGWTTYFK GFHENRKNVY SSNDIPTSII | |
| tularensis] | YRIVDDNLPK FLENKAKYES LKDKAPEAIN YEQIKKDLAE ELTFDIDYKT | |
| | SEVNQRVFSL DEVFEIANFN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLFDD LKAQKLDLSK IYFKNDKSLT | |
| | DLSQQVFDDY SVIGTAVLEY ITQQVAPKNL DNPSKKEQDL IAKKTEKAKY | |
| | LSLETIKLAL EEFNKHRDID KQCRFEEILA NFAAIPMIFD EIAQNKDNLA | |
| | QISIKYQNQG KKDLLQASAE DDVKAIKDLL DQTNNLLHRL KIFHISQSED | |
| | KANILDKDEH FYLVFEECYF ELANIVPLYN KIRNYITQKP YSDEKFKLNF | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GNPQKGYEKF EFNIEDCRKF IDFYKESISK HPEWKDFGFR FSDTQRYNSI | |
| | DEFYREVENQ GYKLTFENIS ESYIDSVVNQ GKLYLFQIYN KDFSAYSKGR | |
| | PNLHTLYWKA LFDERNLQDV VYKLNGEAEL FYRKKSIPKK ITHPAKEAIA | |
| | NKNKDNPKKE SFFEYDLIKD KRFTEDKFFF HCPITINFKS SGANKFNDEI | |
| | NLLLKEKAND VHILSIDRGE RHLAYYTLVD GKGNIIKQDT FNIIGNDRMK | |
| | TNYHDKLAAI EKDRDSARKD WKKINNIKEM KEGYLSQVVH EIAKLVIEHN | |
| | AIVVFEDLNF GFKRGRFKVE KQVYQKLEKM LIEKLNYLVF KDNEFDKTGG | |
| | VLRAYQLTAP FETFKKMGKQ TGIIYYVPAG FTSKICPVTG FVNQLYPKYE | |
| | SVSKSQEFFS KFDKICYNLD KGYFEFSFDY KNFGDKAAKG KWTIASFGSR | |
| | LINFRNSDKN HNWDTREVYP TKELEKLLKD YSIEYGHGEC IKAAICGESD | |
| | KKFFAKLTSI LNTILQMRNS KTGTELDYLI SPVADVNGNF FDSRQAPKNM | |
| | PQDADANGAY HIGLKGLMLL DRIKNNQEGK KLNLVIKNEE YFEFVQNRNN | |
| WP_003034647 | MSIYQEFVNK YSLSKTLRFE LIPQGKTLEN IKARGLILDD EKRAKDYKKA | (SEQ ID |
| type V CRISPR- | KQIIDKYHQF FIEEILSSVC ISEDLLQNYS DVYFKLKKSD DDNLQKDFKS | NO: 129) |
| associated protein | AKDTIKKQIS EYIKDSEKFK NLFNQNLIDA KKGQESDLIL WLKQSKDNGI | |
| Cpf1 [Francisella | ELFKANSDIT DIDEALEIIK SFKGWTTYFK GFHENRKNVY SSDDIPTSII | |
| tularensis] | YRIVDDNLPK FLENKAKYES LKDKAPEAIN YEQIKKDLAE ELTFDIDYKT | |
| | SEVNQRVFSL DEVFEIANFN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLFDD LKAQKLDLSK IYFKNDKSLT | |
| | DLSQQVFDDY SVIGTAVLEY ITQQVAPKNL DNPSKKEQDL IAKKTEKAKY | |
| | LSLETIKLAL EEFNKHRDID KQCRFEEILA NFAAIPMIFD EIAQNKDNLA | |
| | QISLKYQNQG KKDLLQASAE EDVKAIKDLL DQTNNLLHRL KIFHISQSED | |
| | KANILDKDEH FYLVFEECYF ELANIVPLYN KIRNYITQKP YSDEKFKLNF | |
| | ENSTLANGWI KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GNPQKGYEKF EFNIEDCRKF IDFYKESISK HPEWKDFGFR FSDTQRYNSI | |
| | DEFYREVENQ GYKLTFENIS ESYIDSVVNQ GKLYLFQIYN KDFSAYSKGR | |
| | PNLHTLYWKA LFDERNLQDV VYKLNGEAEL FYRKQSIPKK ITHPAKEAIA | |
| | NKNKDNPKKE SVFEYDLIKD KRFTEDKFFF HCPITINFKS SGANKFNDEI | |
| | NLLLKEKAND VHILSIDRGE RHLAYYTLVD GKGNIIKQDT FNIIGNDRMK | |
| | TNYHDKLAAI EKDRDSARKD WKKINNIKEM KEGYLSQVVH EIAKLVIEHN | |
| | AIVVFEDLNF GFKRGRFKVE KQVYQKLEKM LIEKLNYLVF KDNEFDKTGG | |
| | VLRAYQLTAP FETFKKMGKQ TGIIYYVPAG FTSKICPVTG FVNQLYPKYE | |
| | SVSKSQEFFS KFDKICYNLD KGYFEFSFDY KNFGDKAAKG KWTIASFGSR | |
| | LINFRNSDKN HNWDTREVYP TKELEKLLKD YSIEYGHGEC IKAAICGESD | |
| | KKFFAKLTSV LNTILQMRNS KTGTELDYLI SPVADVNGNF FDSRQAPKNM | |
| | PQDADANGAY HIGLKGLMLL DRIKNNQEGK KLNLVIKNEE YFEFVQNRNN | |
| WP_003040289.1 | MSIYQEFVNK YSLSKTLRFE LIPQGKTLEN IKARGLILDD EKRAKDYKKA | (SEQ ID |
| type V CRISPR- | KQIIDKYHQF FIEEILSSVC ISEDLLQNYS DVYFKLKKSD DDNLQKDFKS | NO: 130) |
| associated protein | AKDTIKKQIS EYIKDSEKFK NLFNQNLIDA KKGQESDLIL WLKQSKDNGI | |
| Cpf1 [Francisella | ELFKANSDIT DIDEALEIIK SFKGWTTYFK GFHENRKNVY SSNDIPTSII | |
| tularensis subsp. | YRIVDDNLPK FLENKAKYES LKDKAPEAIN YEQIKKDLAE ELTFDIDYKT | |
| novicida U112] | SEVNQRVFSL DEVFEIANFN NYLNQSGITK FNTIIGGKFV NGENTKRKGI | |
| | NEYINLYSQQ INDKTLKKYK MSVLFKQILS DTESKSFVID KLEDDSDVVT | |
| | TMQSFYEQIA AFKTVEEKSI KETLSLLFDD LKAQKLDLSK IYFKNDKSLT | |
| | DLSQQVFDDY SVIGTAVLEY ITQQIAPKNL DNPSKKEQEL IAKKTEKAKY | |
| | LSLETIKLAL EEFNKHRDID KQCRFEEILA NFAAIPMIFD EIAQNKDNLA | |
| | QISIKYQNQG KKDLLQASAE DDVKAIKDLL DQTNNLLHKL KIFHISQSED | |
| | KANILDKDEH FYLVFEECYF ELANIVPLYN KIRNYITQKP YSDEKFKLNF | |
| | ENSTLANGWD KNKEPDNTAI LFIKDDKYYL GVMNKKNNKI FDDKAIKENK | |
| | GEGYKKIVYK LLPGANKMLP KVFFSAKSIK FYNPSEDILR IRNHSTHTKN | |
| | GSPQKGYEKF EFNIEDCRKF IDFYKQSISK HPEWKDFGFR FSDTQRYNSI | |
| | DEFYREVENQ GYKLTFENIS ESYIDSVVNQ GKLYLFQIYN KDFSAYSKGR | |
| | PNLHTLYWKA LFDERNLQDV VYKLNGEAEL FYRKQSIPKK ITHPAKEAIA | |
| | NKNKDNPKKE SVFEYDLIKD KRFTEDKFFF HCPITINFKS SGANKFNDEI | |
| | NLLLKEKAND VHILSIDRGE RHLAYYTLVD GKGNIIKQDT FNIIGNDRMK | |
| | TNYHDKLAAI EKDRDSARKD WKKINNIKEM KEGYLSQVVH EIAKLVIEYN | |
| | AIVVFEDLNF GFKRGRFKVE KQVYQKLEKM LIEKLNYLVF KDNEFDKTGG | |
| | VLRAYQLTAP FETFKKMGKQ TGIIYYVPAG FTSKICPVTG FVNQLYPKYE | |
| | SVSKSQEFFS KFDKICYNLD KGYFEFSFDY KNFGDKAAKG KWTIASFGSR | |
| | LINFRNSDKN HNWDTREVYP TKELEKLLKD YSIEYGHGEC IKAAICGESD | |
| | KKFFAKLTSV LNTILQMRNS KTGTELDYLI SPVADVNGNF FDSRQAPKNM | |
| | PQDADANGAY HIGLKGLMLL GRIKNNQEGK KLNLVIKNEE YFEFVQNRNN | |
| KKQ38174 | MKSFDSFTNL YSLSKTLKFE MRPVGNTQKM LDNAGVFEKD KLIQKKYGKT | (SEQ ID |
| hypothetical protein | KPYFDRLHRE FIEEALTGVE LIGLDENFRT LVDWQKDKKN NVAMKAYENS | NO: 131) |
| US54_C0016G0015 | LQRLRTEIGK IFNLKAEDWV KNKYPILGLK NKNTDILFEE AVFGILKARY | |
| [Candidatus | GEEKDTFIEV EEIDKTGKSK INQISIFDSW KGFTGYFKKF FETRKNFYKN | |
| Roizmanbacteria | DGTSTAIATR IIDQNLKRFI DNLSIVESVR QKVDLAETEK SFSISLSQFF | |
| bacterium | SIDFYNKCLL QDGIDYYNKI IGGETLKNGE KLIGLNELIN QYRQNNKDQK | |
| GW2011_GWA2_37_7] | IPFFKLLDKQ ILSEKILFLD EIKNDTELIE ALSQFAKTAE EKTKIVKKLF | |
| | ADFVENNSKY DLAQIYISQE AFNTISNKWT SETETFAKYL FEAMKSGKLA | |
| | KYEKKDNSYK FPDFIALSQM KSALLSISLE GHFWKEKYYK ISKFQEKTNW | |
| | EQFLAIFLYE FNSLFSDKIN TKDGETKQVG YYLFAKDLHN LILSEQIDIP | |
| | KDSKVTIKDF ADSVLTIYQM AKYFAVEKKR AWLAEYELDS FYTQPDTGYL | |
| | QFYDNAYEDI VQVYNKLRNY LTKKPYSEEK WKLNFENSTL ANGWDKNKES | |
| | DNSAVILQKG GKYYLGLITK GHNKIFDDRF QEKFIVGIEG GKYEKIVYKF | |
| | FPDQAKMFPK VCFSAKGLEF FRPSEEILRI YNNAEFKKGE TYSIDSMQKL | |
| | IDFYKDCLTK YEGWACYTFR HLKPTEEYQN NIGEFFRDVA EDGYRIDFQG | |
| | ISDQYIHEKN EKGELHLFEI HNKDWNLDKA RDGKSKTTQK NLHTLYFESL | |
| | FSNDNVVQNF PIKLNGQAEI FYRPKTEKDK LESKKDKKGN KVIDHKRYSE | |
| | NKIFFHVPLT LNRTKNDSYR FNAQINNFLA NNKDINIIGV DRGEKHLVYY | |
| | SVITQASDIL ESGSLNELNG VNYAEKLGKK AENREQARRD WQDVQGIKDL | |
| | KKGYISQVVR KLADLAIKHN AIIILEDLNM RFKQVRGGIE KSIYQQLEKA | |
| | LIDKLSFLVD KGEKNPEQAG HLLKAYQLSA PFETFQKMGK QTGIIFYTQA | |
| | SYTSKSDPVT GWRPHLYLKY FSAKKAKDDI AKFTKIEFVN DRFELTYDIK | |
| | DFQQAKEYPN KTVWKVCSNV ERFRWDKNLN QNKGGYTHYT NITENIQELF | |
| | TKYGIDITKD LLTQISTIDE KQNTSFFRDF IFYFNLICQI RNTDDSEIAK | |
| | KNGKDDFILS PVEPFFDSRK DNGNKLPENG DDNGAYNIAR KGIVILNKIS | |
| | QYSEKNENCE KMKWGDLYVS NIDWDNFVTQ ANARH | |
| WP_022097749 | MNGNRSIVYR EFVGVTPVAK TLRNELRPVG HTQEHIIQNG LIQEDELRQE | (SEQ ID |
| type V CRISPR- | KSTELKNIMD DYYREYIDKS LSGLTDLDFT LLFELMNSVQ SSLSKDNKKA | NO: 132) |
| associated protein | LEKEHNKMRE QICTHLQSDS DYKNMFNAKL FKEILPDFIK NYNQYDVKDK | |
| Cpf1 [Eubacterium | AGKLETLALF NGFSTYFTDF FEKRKNVFTK EAVSTSIAYR IVHENSLIFL | |
| eligens CAG:72] | ANMTSYKKIS EKALDEIEVI EKNNQDKMGD WELNQIFNPD FYNMVLIQSG | |
| | IDFYNEICGV VNAHMNLYCQ QTKNNYNLFK MRKLHKQILA YTSTSFEVPK | |
| | MFEDDMSVYN AVNAFIDETE KGNIIGKLKD IVNKYDELDE KRIYISKDFY | |
| | ETLSCFMSGN WNLITGCVEN FYDENIHAKG KSKEEKVKKA VKEDKYKSIN | |
| | DVNDLVEKYI DEKERNEFKN SNAKQYIREI SNIITDTETA HLEYDEHISL | |
| | IESEEKADEI KKRLDMYMNM YHWVKAFIVD EVLDRDEMFY SDIDDIYNIL | |
| | ENIVPLYNRV RNYVTQKPYT SKKIKLNFQS PTLANGWSQS KEFDNNAIIL | |
| | IRDNKYYLAI FNAKNKPDKK IIQGNSDKKN DNDYKKMVYN LLPGANKMLP | |
| | KVFLSKKGIE TFKPSDYIIS GYNAHKHIKT SENFDISFCR DLIDYFKNSI | |
| | EKHAEWRKYE FKFSATDSYN DISEFYREVE MQGYRIDWTY ISEADINKLD | |
| | EEGKIYLFQI YNKDFAENST GKENLHTMYF KNIFSEENLK NIVIKINGQA | |
| | ELFYRKASVK NPVKHKKDSV LVNKTYKNQL DNGDVVRIPI PDDIYNEIYK | |
| | MYNGYIKESD LSEAAKEYLD KVEVRTAQKD IVKDYRYTVD KYFIHTPITI | |
| | NYKVTARNNV NDMAVKYIAQ NDDIHVIGID RGERNLIYIS VIDSHGNIVK | |
| | QKSYNILNNY DYKKKLVEKE KTREYARKNW KSIGNIKELK EGYISGVVHE | |
| | IAMLMVEYNA IIAMEDLNYG FKRGRFKVER QVYQKFESML INKLNYFASK | |
| | GKSVDEPGGL LKGYQLTYVP DNIKNLGKQC GVIFYVPAAF TSKIDPSTGF | |
| | ISAFNFKSIS TNASRKQFFM QFDEIRYCAE KDMFSFGFDY NNFDTYNITM | |
| | GKTQWTVYTN GERLQSEFNN ARRTGKTKSI NLTETIKLLL EDNEINYADG | |
| | HDVRIDMEKM YEDKNSEFFA QLLSLYKLTV QMRNSYTEAE EQEKGISYDK | |
| | IISPVINDEG EFFDSDNYKE SDDKECKMPK DADANGAYCI ALKGLYEVLK | |
| | IKSEWTEDGF DRNCLKLPHA EWLDFIQNKR YE | |
| WP_021739647 | MIKKTIDTVL NVRPIFVGIQ HLYFYEGPCR FGEGDELMPE YDAMMNQEMN | (SEQ ID |
| hypothetical protein | AAYVNEVVQH ETEGVHIMDP IYVERDDWFR SPEAMYEKMA EDIDKVDFYL | NO: 133) |
| [Eubacterium | FHFGIGRGDI YLEFAERYKK PVGAAPGLCC DGIGNTAAVK NRGLEAYAFM | |
| ramulus] | SWDEFDTWMR VLRVRKCLKN TRVLLAVRWD SNRSYSSYDN FINQSDVTNK | |
| | WGIQFRHVNV HELLDQTHPV DPTTNPSTPG RKALNINDED MKEIEKITDE | |
| | LIANAEACTM EPDMVKKTIQ AYYTVQKLLD AYDCNAFTAP CPDLCSTRRF | |
| | SEEKFTLCMT HSLNDENGIS SACEYDINSV IGKVIMTNLS GKAPYMGNTN | |
| | AIVFDKEGHM IPFHKFNDNT IEDIADKTNL YMTFHSTPNR NLKGLKAEKE | |
| | RYRLAPFAYS GFGATIRYDF AQDIGQVITM IRISPDATKI FIAKGTISGG | |
| | AGYEMKNCDQ GVFFNVADKV DFYHKQQYFG NHTVLAYGDY VEELKMLAEA | |
| | LGIEAVIA | |
| gi\|800943167 | MKNFSNLYQV SKTVRFELKP IGNTLENIKN KSLLKNDSIR AESYQKMKKT | (SEQ ID |
| WP_045971446.1 | IDEFHKYFID LALNNKKLSY LNEYIALYTQ SAEAKKEDKF KADFKKVQDN | NO: 134) |
| type V CRISPR- | LRKEIVSSFT EGEAKAIFSV LDKKELITIE LEKWKNENNL AVYLDESFKS | |
| associated protein | FTTYFTGFHQ NRKNMYSAEA NSTAIAYRLI HENLPKFIEN SKAFEKSSQI | |
| Cpf1 | AELQPKIEKL YKEFEAYLNV NSISELFEID YFNEVLTQKG ITVYNNIIGG | |
| [Flavobacterium sp. | RTATEGKQKI QGLNEIINLY NQTKPKNERL PKLKQLYKQI LSDRISLSFL | |
| 316] | PDAFTEGKQV LKAVFEFYKI NLLSYKQDGV EESQNLLELI QQVVKNLGNQ | |
| | DVNKIYLKND TSLTTIAQQL FGDFSVFSAA LQYRYETVVN PKYTAEYQKA | |
| | NEAKQEKLDK EKIKFVKQDY FSIAFLQEVV ADYVKTLDEN LDWKQKYTPS | |
| | CIADYFTTHF IAKKENEADK TFNFIANIKA KYQCIQGILE QADDYEDELK | |
| | QDQKLIDNIK FFLDAILEVV HFIKPLHLKS ESITEKDNAF YDVFENYYEA | |
| | LNVVTPLYNM VRNYVTQKPY STEKIKLNFE NAQLLNGWDA NKEKDYLTTI | |
| | LKRDGNYFLA IMDKKHNKTF QQFTEDDENY EKIVYKLLPG VNKMLPKVFF | |
| | SNKNIAFFNP SKEILDNYKN NTHKKGATFN LKDCHALIDF FKDSLNKHED | |
| | WKYFDFQFSE TKTYQDLSGF YKEVEHQGYK INFKKVSVSQ IDTLIEEGKM | |
| | YLFQIYNKDF SPYAKGKPNM HTLYWKALFE TQNLENVIYK LNGQAEIFFR | |
| | KASIKKKNII THKAHQPIAA KNPLTPTAKN TFAYDLIKDK RYTVDKFQFH | |
| | VPITMNFKAT GNSYINQDVL AYLKDNPEVN IIGLDRGERH LVYLTLIDQK | |
| | GTILLQESLN VIQDEKTHTP YHTLLDNKEI ARDKARKNWG SIESIKELKE | |
| | GYISQVVHKI TKMMIEHNAI VVMEDLNFGF KRGRFKVEKQ IYQKLEKMLI | |
| | DKLNYLVLKD KQPHELGGLY NALQLTNKFE SFQKMGKQSG FLFYVPAWNT | |
| | SKIDPTTGFV NYFYTKYENV EKAKTFFSKF DSILYNKTKG YFEFVVKNYS | |
| | DFNPKAADTR QEWTICTHGE RIETKRQKEQ NNNFVSTTIQ LTEQFVNFFE | |
| | KVGLDLSKEL KTQLIAQNEK SFFEELFHLL KLTLQMRNSE SHTEIDYLIS | |
| | PVANEKGIFY DSRKATASLP IDADANGAYH IAKKGLWIME QINKTNSEDD | |
| | LKKVKLAISN REWLQYVQQV QKK | |
| WP_044110123.1 | MKQFTNLYQL SKTLRFELKP IGKTLEHINA NGFIDNDAHR AESYKKVKKL | (SEQ ID |
| type V CRISPR- | IDDYHKDYIE NVLNNFKLNG EYLQAYFDLY SQDTKDKQFK DIQDKLRKSI | NO: 135) |
| associated protein | ASALKGDDRY KTIDKKELIR QDMKTFLKKD TDKALLDEFY EFTTYFTGYH | |
| Cpf1 [Prevotella | ENRKNMYSDE AKSTAIAYRL IHDNLPKFID NIAVFKKIAN TSVADNFSTI | |
| brevis] | YKNFEEYLNV NSIDEIFSLD YYNIVLTQTQ IEVYNSIIGG RTLEDDTKIQ | |
| | GINEFVNLYN QQLANKKDRL PKLKPLFKQI LSDRVQLSWL QEEFNTGADV | |
| | LNAVKEYCTS YFDNVEESVK VLLTGISDYD LSKIYITNDL ALTDVSQRMF | |
| | GEWSIIPNAI EQRLRSDNPK KTNEKEEKYS DRISKLKKLP KSYSLGYINE | |
| | CISELNGIDI ADYYATLGAI NTESKQEPSI PTSIQVHYNA LKPILDTDYP | |
| | REKNLSQDKL TVMQLKDLLD DFKALQHFIK PLLGNGDEAE KDEKFYGELM | |
| | QLWEVIDSIT PLYNKVRNYC TRKPFSTEKI KVNFENAQLL DGWDENKEST | |
| | NASIILRKNG MYYLGIMKKE YRNILTKPMP SDGDCYDKVV YKFFKDITTM | |
| | VPKCTTQMKS VKEHFSNSND DYTLFEKDKF IAPVVITKEI FDLNNVLYNG | |
| | VKKFQIGYLN NTGDSFGYNH AVEIWKSFCL KFLKAYKSTS IYDFSSIEKN | |
| | IGCYNDLNSF YGAVNLLLYN LTYRKVSVDY IHQLVDEDKM YLFMIYNKDF | |
| | STYSKGTPNM HTLYWKMLFD ESNLNDVVYK LNGQAEVFYR KKSITYQHPT | |
| | HPANKPIDNK NVNNPKKQSN FEYDLIKDKR YTVDKFMFHV PITLNFKGMG | |
| | NGDINMQVRE YIKTTDDLHF IGIDRGERHL LYICVINGKG EIVEQYSLNE | |
| | IVNNYKGTEY KTDYHTLLSE RDKKRKEERS SWQTIEGIKE LKSGYLSQVI | |
| | HKITQLMIKY NAIVLLEDLN MGFKRGRQKV ESSVYQQFEK ALIDKLNYLV | |
| | DKNKDANEIG GLLHAYQLTN DPKLPNKNSK QSGFLFYVPA WNTSKIDPVT | |
| | GFVNLLDTRY ENVAKAQAFF KKFDSIRYNK EYDRFEFKFD YSNFTAKAED | |
| | TRTQWTLCTY GTRIETFRNA EKNSNWDSRE IDLTTEWKTL FTQHNIPLNA | |
| | NLKEAILLQA NKNFYTDILH LMKLTLQMRN SVTGTDIDYM VSPVANECGE | |
| | FFDSRKVKEG LPVNADANGA YNIARKGLWL AQQIKNANDL SDVKLAITNK | |
| | EWLQFAQKKQ YLKD | |
| WP_036388671.1 | MLFQDFTHLY PLSKTMRFEL KPIGKTLEHI HAKNFLSQDE TMADMYQKVK | (SEQ ID |
| type V CRISPR- | AILDDYHRDF IADMMGEVKL TKLAEFYDVY LKFRKNPKDD GLQKQLKDLQ | NO: 136) |
| associated protein | AVLRKEIVKP IGNGGKYKAG YDRLFGAKLF KDGKELGDLA KFVIAQEGES | |
| Cpf1 [Moraxella | SPKLAHLAHF EKFSTYFTGF HDNRKNMYSD EDKHTAITYR LIHENLPRFI | |
| caprae] | DNLQILATIK QKHSALYDQI INELTASGLD VSLASHLDGY HKLLTQEGIT | |
| | AYNTLLGGIS GEAGSRKIQG INELINSHHN QHCHKSERIA KLRPLHKQIL | |
| | SDGMGVSFLP SKFADDSEMC QAVNEFYRHY ADVFAKVQSL FDGFDDHQKD | |
| | GIYVEHKNLN ELSKQAFGDF ALLGRVLDGY YVDVVNPEFN ERFAKAKTDN | |
| | AKAKLTKEKD KFIKGVHSLA SLEQAIEHYT ARHDDESVQA GKLGQYFKHG | |
| | LAGVDNPIQK IHNNHSTIKG FLERERPAGE RALPKIKSGK NPEMTQLRQL | |
| | KELLDNALNV AHFAKLLTTK TTLDNQDGNF YGEFGALYDE LAKIPTLYNK | |
| | VRDYLSQKPF STEKYKLNFG NPTLLNGWDL NKEKDNFGII LQKDGCYYLA | |
| | LLDKAHKKVF DNAPNTGKNV YQKMIYKLLP GPNKMLPKVF FAKSNLDYYN | |
| | PSAELLDKYA QGTHKKGNNF NLKDCHALID FFKAGINKHP EWQHFGFKFS | |
| | PTSSYQDLSD FYREVEPQGY QVKFVDINAD YINELVEQGQ LYLFQIYNKD | |
| | FSPKAHGKPN LHTLYFKALF SKDNLANPIY KLNGEAQIFY RKASLDMNET | |
| | TIHRAGEVLE NKNPDNPKKR QFVYDIIKDK RYTQDKFMLH VPITMNFGVQ | |
| | GMTIKEFNKK VNQSIQQYDE VNVIGIDRGE RHLLYLTVIN SKGEILEQRS | |
| | LNDITTASAN GTQMTTPYHK ILDKREIERL NARVGWGEIE TIKELKSGYL | |
| | SHVVHQISQL MLKYNAIVVL EDLNFGFKRG RFKVEKQIYQ NFENALIKKL | |
| | NHLVLKDEAD DEIGSYKNAL QLTNNFTDLK SIGKQTGFLF YVPAWNTSKI | |
| | DPETGFVDLL KPRYENIAQS QAFFGKFDKI CYNADKDYFE FHIDYAKFTD | |
| | KAKNSRQIWK ICSHGDKRYV YDKTANQNKG ATKGINVNDE LKSLFARHHI | |
| | NDKQPNLVMD ICQNNDKEFH KSLIYLLKTL LALRYSNASS DEDFILSPVA | |
| | NDEGMFFNSA LADDTQPQNA DANGAYHIAL KGLWVLEQIK NSDDLNKVKL | |
| | AIDNQTWLNF AQNR | |
| WP_020988726.1 | MEDYSGFVNI YSIQKTLRFE LKPVGKTLEH IEKKGFLKKD KIRAEDYKAV | (SEQ ID |
| type V CRISPR- | KKIIDKYHRA YIEEVFDSVL HQKKKKDKTR FSTQFIKEIK EFSELYYKTE | NO: 137) |
| associated protein | KNIPDKERLE ALSEKLRKML VGAFKGEFSE EVAEKYKNLF SKELIRNEIE | |
| Cpf1 [Leptospira | KFCETDEERK QVSNFKSFTT YFTGFHSNRQ NIYSDEKKST AIGYRIIHQN | |
| inadai] | LPKFLDNLKI IESIQRRFKD FPWSDLKKNL KKIDKNIKLT EYFSIDGFVN | |
| | VLNQKGIDAY NTILGGKSEE SGEKIQGLNE YINLYRQKNN IDRKNLPNVK | |
| | ILFKQILGDR ETKSFIPEAF PDDQSVLNSI TEFAKYLKLD KKKKSIIAEL | |
| | KKFLSSFNRY ELDGIYLAND NSLASISTFL FDDWSFIKKS VSFKYDESVG | |
| | DPKKKIKSPL KYEKEKEKWL KQKYYTISFL NDAIESYSKS QDEKRVKIRL | |
| | EAYFAEFKSK DDAKKQFDLL ERIEEAYAIV EPLLGAEYPR DRNLKADKKE | |
| | VGKIKDFLDS IKSLQFFLKP LLSAEIFDEK DLGFYNQLEG YYEEIDSIGH | |
| | LYNKVRNYLT GKIYSKEKFK LNFENSTLLK GWDENREVAN LCVIFREDQK | |
| | YYLGVMDKEN NTILSDIPKV KPNELFYEKM VYKLIPTPHM QLPRIIFSSD | |
| | NLSIYNPSKS ILKIREAKSF KEGKNFKLKD CHKFIDFYKE SISKNEDWSR | |
| | FDFKFSKTSS YENISEFYRE VERQGYNLDF KKVSKFYIDS LVEDGKLYLF | |
| | QIYNKDFSIF SKGKPNLHTI YFRSLFSKEN LKDVCLKLNG EAEMFFRKKS | |
| | INYDEKKKRE GHHPELFEKL KYPILKDKRY SEDKFQFHLP ISLNFKSKER | |
| | LNFNLKVNEF LKRNKDINII GIDRGERNLL YLVMINQKGE ILKQTLLDSM | |
| | QSGKGRPEIN YKEKLQEKEI ERDKARKSWG TVENIKELKE GYLSIVIHQI | |
| | SKLMVENNAI VVLEDLNIGF KRGRQKVERQ VYQKFEKMLI DKLNFLVFKE | |
| | NKPTEPGGVL KAYQLTDEFQ SFEKLSKQTG FLFYVPSWNT SKIDPRTGFI | |
| | DFLHPAYENI EKAKQWINKF DSIRFNSKMD WFEFTADTRK FSENLMLGKN | |
| | RVWVICTTNV ERYFTSKTAN SSIQYNSIQI TEKLKELFVD IPFSNGQDLK | |
| | PEILRKNDAV FFKSLLFYIK TTLSLRQNNG KKGEEEKDFI LSPVVDSKGR | |
| | FFNSLEASDD EPKDADANGA YHIALKGLMN LLVLNETKEE NLSRPKWKIK | |
| | NKDWLEFVWE RNR | |
| WP_023936172.1 | MPWIDLKDFT NLYPVSKTLR FELKPVGKTL ENIEKAGILK EDEHRAESYR | (SEQ ID |
| type V CRISPR- | RVKKIIDTYH KVFIDSSLEN MAKMGIENEI KAMLQSFCEL YKKDHRTEGE | NO: 138) |
| associated protein | DKALDKIRAV LRGLIVGAFT GVCGRRENTV QNEKYESLFK EKLIKEILPD | |
| Cpf1 | FVLSTEAESL PFSVEEATRS LKEFDSFTSY FAGFYENRKN IYSTKPQSTA | |
| [Porphyromonas | IAYRLIHENL PKFIDNILVF QKIKEPIAKE LEHIRADFSA GGYIKKDERL | |
| crevioricanis] | EDIFSLNYYI HVLSQAGIEK YNALIGKIVT EGDGEMKGLN EHINLYNQQR | |
| | GREDRLPLFR PLYKQILSDR EQLSYLPESF EKDEELLRAL KEFYDHIAED | |
| | ILGRTQQLMT SISEYDLSRI YVRNDSQLTD ISKKMLGDWN AIYMARERAY | |
| | DHEQAPKRIT AKYERDRIKA LKGEESISLA NLNSCIAFLD NVRDCRVDTY | |
| | LSTLGQKEGP HGLSNLVENV FASYHEAEQL LSFPYPEENN LIQDKDNVVL | |
| | IKNLLDNISD LQRFLKPLWG MGDEPDKDER FYGEYNYIRG ALDQVIPLYN | |
| | KVRNYLTRKP YSTRKVKLNF GNSQLLSGWD RNKEKDNSCV ILRKGQNFYL | |
| | AIMNNRHKRS FENKVLPEYK EGEPYFEKMD YKFLPDPNKM LPKVFLSKKG | |
| | IEIYEPSPKL LEQYGHGTHK KGDTFSMDDL HELIDFFKHS IEAHEDWKQF | |
| | GFKFSDTATY ENVSSFYREV EDQGYKLSFR KVSESYVYSL IDQGKLYLFQ | |
| | IYNKDFSPCS KGTPNLHTLY WRMLFDERNL ADVIYKLDGK AEIFFREKSL | |
| | KNDHPTHPAG KPIKKKSRQK KGEESLFEYD LVKDRRYTMD KFQFHVPITM | |
| | NFKCSAGSKV NDMVNAHIRE AKDMHVIGID RGERNLLYIC VIDSRGTILD | |
| | QISLNTINDI DYHDLLESRD KDRQQERRNW QTIEGIKELK QGYLSQAVHR | |
| | IAELMVAYKA VVALEDLNMG FKRGRQKVES SVYQQFEKQL IDKLNYLVDK | |
| | KKRPEDIGGL LRAYQFTAPF KSFKEMGKQN GFLFYIPAWN TSNIDPTTGF | |
| | VNLFHAQYEN VDKAKSFFQK FDSISYNPKK DWFEFAFDYK NFTKKAEGSR | |
| | SMWILCTHGS RIKNFRNSQK NGQWDSEEFA LTEAFKSLFV RYEIDYTADL | |
| | KTAIVDEKQK DFFVDLLKLF KLTVQMRNSW KEKDLDYLIS PVAGADGRFF | |
| | DTREGNKSLP KDADANGAYN IALKGLWALR QIRQTSEGGK LKLAISNKEW | |
| | LQFVQERSYE KD | |
| WP_009217842.1 | MRKFNEFVGL YPISKTLRFE LKPIGKTLEH IQRNKLLEHD AVRADDYVKV | (SEQ ID |
| type V CRISPR- | KKIIDKYHKC LIDEALSGFT FDTEADGRSN NSLSEYYLYY NLKKRNEQEQ | NO: 139) |
| associated protein | KTFKTIQNNL RKQIVNKLTQ SEKYKRIDKK ELITTDLPDF LTNESEKELV | |
| Cpf1 [Bacteroidetes | EKFKNFTTYF TEFHKNRKNM YSKEEKSTAI AFRLINENLP KFVDNIAAFE | |
| oral taxon 274] | KVVSSPLAEK INALYEDFKE YLNVEEISRV FRLDYYDELL TQKQIDLYNA | |
| | IVGGRTEEDN KIQIKGLNQY INEYNQQQTD RSNRLPKLKP LYKQILSDRE | |
| | SVSWLPPKFD SDKNLLIKIK ECYDALSEKE KVFDKLESIL KSLSTYDLSK | |
| | IYISNDSQLS YISQKMFGRW DIISKAIRED CAKRNPQKSR ESLEKFAERI | |
| | DKKLKTIDSI SIGDVDECLA QLGETYVKRV EDYFVAMGES EIDDEQTDTT | |
| | SFKKNIEGAY ESVKELLNNA DNITDNNLMQ DKGNVEKIKT LLDAIKDLQR | |
| | FIKPLLGKGD EADKDGVFYG EFTSLWTKLD QVTPLYNMVR NYLTSKPYST | |
| | KKIKLNFENS TLMDGWDLNK EPDNTTVIFC KDGLYYLGIM GKKYNRVFVD | |
| | REDLPHDGEC YDKMEYKLLP GANKMLPKVF FSETGIQRFL PSEELLGKYE | |
| | RGTHKKGAGF DLGDCRALID FFKKSIERHD DWKKFDFKFS DTSTYQDISE | |
| | FYREVEQQGY KMSFRKVSVD YIKSLVEEGK LYLFQIYNKD FSAHSKGTPN | |
| | MHTLYWKMLF DEENLKDVVY KLNGEAEVFF RKSSITVQSP THPANSPIKN | |
| | KNKDNQKKES KFEYDLIKDR RYTVDKFLFH VPITMNFKSV GGSNINQLVK | |
| | RHIRSATDLH IIGIDRGERH LLYLTVIDSR GNIKEQFSLN EIVNEYNGNT | |
| | YRTDYHELLD TREGERTEAR RNWQTIQNIR ELKEGYLSQV IHKISELAIK | |
| | YNAVIVLEDL NFGFMRSRQK VEKQVYQKFE KMLIDKLNYL VDKKKPVAET | |
| | GGLLRAYQLT GEFESFKTLG KQSGILFYVP AWNTSKIDPV TGFVNLFDTH | |
| | YENIEKAKVF FDKFKSIRYN SDKDWFEFVV DDYTRFSPKA EGTRRDWTIC | |
| | TQGKRIQICR NHQRNNEWEG QEIDLTKAFK EHFEAYGVDI SKDLREQINT | |
| | QNKKEFFEEL LRLLRLTLQM RNSMPSSDID YLISPVANDT GCFFDSRKQA | |
| | ELKENAVLPM NADANGAYNI ARKGLLAIRK MKQEENDSAK ISLAISNKEW | |
| | LKFAQTKPYL ED | |
| WP_036890108.1 | MDSLKDFTNL YPVSKTLRFE LKPVGKTLEN IEKAGILKED EHRAESYRRV | (SEQ ID |
| type V CRISPR- | KKIIDTYHKV FIDSSLENMA KMGIENEIKA MLQSFCELYK KDHRTEGEDK | NO: 140) |
| associated protein | ALDKIRAVLR GLIVGAFTGV CGRRENTVQN EKYESLFKEK LIKEILPDFV | |
| Cpf1 | LSTEAESLPF SVEEATRSLK EFDSFTSYFA GFYENRKNIY STKPQSTAIA | |
| [Porphyromonas | YRLIHENLPK FIDNILVFQK IKEPIAKELE HIRADFSAGG YIKKDERLED | |
| crevioricanis] | IFSLNYYIHV LSQAGIEKYN ALIGKIVTEG DGEMKGLNEH INLYNQQRGR | |
| | EDRLPLFRPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKMLPEYKEG EPYFEKMDYK FLPDPNKMLP KVFLSKKGIE | |
| | IYKPSPKLLE QYGHGTHKKG DTFSMDDLHE LIDFFKHSIE AHEDWKQFGF | |
| | KFSDTATYEN VSSFYREVED QGYKLSFRKV SESYVYSLID QGKLYLFQIY | |
| | NKDFSPCSKG TPNLHTLYWR MLFDERNLAD VIYKLDGKAE IFFREKSLKN | |
| | DHPTHPAGKP IKKKSRQKKG EESLFEYDLV KDRRYTMDKF QFHVPITMNF | |
| | KCSAGSKVND MVNAHIREAK DMHVIGIDRG ERNLLYICVI DSRGTILDQI | |
| | SLNTINDIDY HDLLESRDKD RQQEHRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGF LFYIPAWNTS NIDPTTGFVN | |
| | LFHVQYENVD KAKSFFQKFD SISYNPKKDW FEFAFDYKNF TKKAEGSRSM | |
| | WILCTHGSRI KNFRNSQKNG QWDSEEFALT EAFKSLFVRY EIDYTADLKT | |
| | AIVDEKQKDF FVDLLKLFKL TVQMRNSWKE KDLDYLISPV AGADGRFFDT | |
| | REGNKSLPKD ADANGAYNIA LKGLWALRQI RQTSEGGKLK LAISNKEWLQ | |
| | FVQERSYEKD | |
| WP_036887416.1 | MDSLKDFTNL YPVSKTLRFE LKPVGKTLEN IEKAGILKED EHRAESYRRV | (SEQ ID |
| type V CRISPR- | KKIIDTYHKV FIDSSLENMA KMGIENEIKA MLQSFCELYK KDHRTEGEDK | NO: 141) |
| associated protein | ALDKIRAVLR GLIVGAFTGV CGRRENTVQN EKYESLFKEK LIKEILPDFV | |
| Cpf1 | LSTEAESLPF SVEEATRSLK EFDSFTSYFA GFYENRKNIY STKPQSTAIA | |
| [Porphyromonas | YRLIHENLPK FIDNILVFQK IKEPIAKELE HIRADFSAGG YIKKDERLED | |
| crevioricanis] | IFSLNYYIHV LSQAGIEKYN ALIGKIVTEG DGEMKGLNEH INLYNQQRGR | |
| | EDRLPLFRPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKVLPEYKEG EPYFEKMDYK FLPDPNKMLP KVFLSKKGIE | |
| | IYKPSPKLLE QYGHGTHKKG DTFSMDDLHE LIDFFKHSIE AHEDWKQFGF | |
| | KFSDTATYEN VSSFYREVED QGYKLSFRKV SESYVYSLID QGKLYLFQIY | |
| | NKDFSPCSKG TPNLHTLYWR MLFDERNLAD VIYKLDGKAE IFFREKSLKN | |
| | DHPTHPAGKP IKKKSRQKKG EESLFEYDLV KDRHYTMDKF QFHVPITMNF | |
| | KCSAGSKVND MVNAHIREAK DMHVIGIDRG ERNLLYICVI DSRGTILDQI | |
| | SLNTINDIDY HDLLESRDKD RQQERRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGF LFYIPAWNTS NIDPTTGFVN | |
| | LFHAQYENVD KAKSFFQKFD SISYNPKKDW FEFAFDYKNF TKKAEGSRSM | |
| | WILCTHGSRI KNFRNSQKNG QWDSEEFALT EAFKSLFVRY EIDYTADLKT | |
| | AIVDEKQKDF FVDLLKLFKL TVQMRNSWKE KDLDYLISPV AGADGRFFDT | |
| | REGNKSLPKD ADANGAYNIA LKGLWALRQI RQTSEGGKLK LAISNKEWLQ | |
| | FVQERSYEKD | |
| WP_023941260.1 | MDSLKDFTNL YPVSKTLRFE LKPVGKTLEN IEKAGILKED EHRAESYRRV | (SEQ ID |
| type V CRISPR- | KKIIDTYHKV FIDSSLENMA KMGIENEIKA MLQSFCELYK KDHRTEGEDK | NO: 142) |
| associated protein | ALDKIRAVLR GLIVGAFTGV CGRRENTVQN EKYESLFKEK LIKEILPDFV | |
| Cpf1 | LSTEAESLPF SVEEATRSLK EFDSFTSYFA GFYENRKNIY STKPQSTAIA | |
| [Porphyromonas | YRLIHENLPK FIDNILVFQK IKEPIAKELE HIRADFSAGG YIKKDERLED | |
| crevioricanis] | IFSLNYYIHV LSQAGIEKYN ALIGKIVTEG DGEMKGLNEH INLYNQQRGR | |
| | EDRLPLFRPL YKQILSDREQ LSYLPESFEK DEELLRALKE FYDHIAEDIL | |
| | GRTQQLMTSI SEYDLSRIYV RNDSQLTDIS KKMLGDWNAI YMARERAYDH | |
| | EQAPKRITAK YERDRIKALK GEESISLANL NSCIAFLDNV RDCRVDTYLS | |
| | TLGQKEGPHG LSNLVENVFA SYHEAEQLLS FPYPEENNLI QDKDNVVLIK | |
| | NLLDNISDLQ RFLKPLWGMG DEPDKDERFY GEYNYIRGAL DQVIPLYNKV | |
| | RNYLTRKPYS TRKVKLNFGN SQLLSGWDRN KEKDNSCVIL RKGQNFYLAI | |
| | MNNRHKRSFE NKVLPEYKEG EPYFEKMDYK FLPDPNKMLP KVFLSKKGIE | |
| | IYKPSPKLLE QYGHGTHKKG DTFSMDDLHE LIDFFKHSIE AHEDWKQFGF | |
| | KFSDTATYEN VSSFYREVED QGYKLSFRKV SESYVYSLID QGKLYLFQIY | |
| | NKDFSPCSKG TPNLHTLYWR MLFDERNLAD VIYKLDGKAE IFFREKSLKN | |
| | DHPTHPAGKP IKKKSRQKKG EESLFEYDLV KDRRYTMDKF QFHVPITMNF | |
| | KCSAGSKVND MVNAHIREAK DMHVIGIDRG ERNLLYICVI DSRGTILDQI | |
| | SLNTINDIDY HDLLESRDKD RQQERRNWQT IEGIKELKQG YLSQAVHRIA | |
| | ELMVAYKAVV ALEDLNMGFK RGRQKVESSV YQQFEKQLID KLNYLVDKKK | |
| | RPEDIGGLLR AYQFTAPFKS FKEMGKQNGF LFYIPAWNTS NIDPTTGFVN | |
| | LFHAQYENVD KAKSFFQKFD SISYNPKKDW FEFAFDYKNF TKKAEGSRSM | |
| | WILCTHGSRI KNFRNSQKNG QWDSEEFALT EAFKSLFVRY EIDYTADLKT | |
| | AIVDEKQKDF FVDLLKLFKL TVQMRNSWKE KDLDYLISPV AGADGRFFDT | |
| | REGNKSLPKD ADANGAYNIA LKGLWALRQI RQTSEGGKLK LAISNKEWLQ | |
| | FVQERSYEKD | |
| WP_037975888.1 | MANSLKDFTN IYQLSKTLRF ELKPIGKTEE HINRKLIIMH DEKRGEDYKS | (SEQ ID |
| type V CRISPR- | VTKLIDDYHR KFIHETLDPA HFDWNPLAEA LIQSGSKNNK ALPAEQKEMR | NO: 143) |
| associated protein | EKIISMFTSQ AVYKKLFKKE LFSELLPEMI KSELVSDLEK QAQLDAVKSF | |
| Cpf1 [Synergistes | DKFSTYFTGF HENRKNIYSK KDTSTSIAFR IVHQNFPKFL ANVRAYTLIK | |
| jonesii] | ERAPEVIDKA QKELSGILGG KTLDDIFSIE SFNNVLTQDK IDYYNQIIGG | |
| | VSGKAGDKKL RGVNEFSNLY RQQHPEVASL RIKMVPLYKQ ILSDRTTLSF | |
| | VPEALKDDEQ AINAVDGLRS ELERNDIFNR IKRLFGKNNL YSLDKIWIKN | |
| | SSISAFSNEL FKNWSFIEDA LKEFKENEFN GARSAGKKAE KWLKSKYFSF | |
| | ADIDAAVKSY SEQVSADISS APSASYFAKF TNLIETAAEN GRKFSYFAAE | |
| | SKAFRGDDGK TEIIKAYLDS LNDILHCLKP FETEDISDID TEFYSAFAEI | |
| | YDSVKDVIPV YNAVRNYTTQ KPFSTEKFKL NFENPALAKG WDKNKEQNNT | |
| | AIILMKDGKY YLGVIDKNNK LRADDLADDG SAYGYMKMNY KFIPTPHMEL | |
| | PKVFLPKRAP KRYNPSREIL LIKENKTFIK DKNFNRTDCH KLIDFFKDSI | |
| | NKHKDWRTFG FDFSDTDSYE DISDFYMEVQ DQGYKLTFTR LSAEKIDKWV | |
| | EEGRLFLFQI YNKDFADGAQ GSPNLHTLYW KAIFSEENLK DVVLKLNGEA | |
| | ELFFRRKSID KPAVHAKGSM KVNRRDIDGN PIDEGTYVEI CGYANGKRDM | |
| | ASLNAGARGL IESGLVRITE VKHELVKDKR YTIDKYFFHV PFTINFKAQG | |
| | QGNINSDVNL FLRNNKDVNI IGIDRGERNL VYVSLIDRDG HIKLQKDFNI | |
| | IGGMDYHAKL NQKEKERDTA RKSWKTIGTI KELKEGYLSQ VVHEIVRLAV | |
| | DNNAVIVMED LNIGFKRGRF KVEKQVYQKF EKMLIDKLNY LVFKDAGYDA | |
| | PCGILKGLQL TEKFESFTKL GKQCGIIFYI PAGYTSKIDP TTGFVNLFNI | |
| | NDVSSKEKQK DFIGKLDSIR FDAKRDMFTF EFDYDKFRTY QTSYRKKWAV | |
| | WTNGKRIVRE KDKDGKFRMN DRLLTEDMKN ILNKYALAYK AGEDILPDVI | |
| | SRDKSLASEI FYVFKNTLQM RNSKRDTGED FIISPVLNAK GRFFDSRKTD | |
| | AALPIDADAN GAYHIALKGS LVLDAIDEKL KEDGRIDYKD MAVSNPKWFE | |
| | FMQTRKFDF | |
| WP_081839471.1 | MENMANSLKD FTNIYQLSKT LRFELKPIGK TEEHINRKLI IMHDEKRGED | (SEQ ID |
| type V CRISPR- | YKSVTKLIDD YHRKFIHETL DPAHFDWNPL AEALIQSGSK NNKALPAEQK | NO: 144) |
| associated protein | EMREKIISMF TSQAVYKKLF KKELFSELLP EMIKSELVSD LEKQAQLDAV | |
| Cpf1 [Synergistes | KSFDKFSTYF TGFHENRKNI YSKKDTSTSI AFRIVHQNFP KFLANVRAYT | |
| jonesii] | LIKERAPEVI DKAQKELSGI LGGKTLDDIF SIESFNNVLT QDKIDYYNQI | |
| | IGGVSGKAGD KKLRGVNEFS NLYRQQHPEV ASLRIKMVPL YKQILSDRTT | |
| | LSFVPEALKD DEQAINAVDG LRSELERNDI FNRIKRLFGK NNLYSLDKIW | |
| | IKNSSISAFS NELFKNWSFI EDALKEFKEN EFNGARSAGK KAEKWLKSKY | |
| | FSFADIDAAV KSYSEQVSAD ISSAPSASYF AKFTNLIETA AENGRKFSYF | |
| | AAESKAFRGD DGKTEIIKAY LDSLNDILHC LKPFETEDIS DIDTEFYSAF | |
| | AEIYDSVKDV IPVYNAVRNY TTQKPFSTEK FKLNFENPAL AKGWDKNKEQ | |
| | NNTAIILMKD GKYYLGVIDK NNKLRADDLA DDGSAYGYMK MNYKFIPTPH | |
| | MELPKVFLPK RAPKRYNPSR EILLIKENKT FIKDKNFNRT DCHKLIDFFK | |
| | DSINKHKDWR TFGFDFSDTD SYEDISDFYM EVQDQGYKLT FTRLSAEKID | |
| | KWVEEGRLFL FQIYNKDFAD GAQGSPNLHT LYWKAIFSEE NLKDVVLKLN | |
| | GEAELFFRRK SIDKPAVHAK GSMKVNRRDI DGNPIDEGTY VEICGYANGK | |
| | RDMASLNAGA RGLIESGLVR ITEVKHELVK DKRYTIDKYF FHVPFTINFK | |
| | AQGQGNINSD VNLFLRNNKD VNIIGIDRGE RNLVYVSLID RDGHIKLQKD | |
| | FNIIGGMDYH AKLNQKEKER DTARKSWKTI GTIKELKEGY LSQVVHEIVR | |
| | LAVDNNAVIV MEDLNIGFKR GRFKVEKQVY QKFEKMLIDK LNYLVFKDAG | |
| | YDAPCGILKG LQLTEKFESF TKLGKQCGII FYIPAGYTSK IDPTTGFVNL | |
| | FNINDVSSKE KQKDFIGKLD SIRFDAKRDM FTFEFDYDKF RTYQTSYRKK | |
| | WAVWTNGKRI VREKDKDGKF RMNDRLLTED MKNILNKYAL AYKAGEDILP | |
| | DVISRDKSLA SEIFYVFKNT LQMRNSKRDT GEDFIISPVL NAKGRFFDSR | |
| | KTDAALPIDA DANGAYHIAL KGSLVLDAID EKLKEDGRID YKDMAVSNPK | |
| | WFEFMQTRKF DF | |
| WP_006283774.1 | MQINNLKIIY MKFTDFTGLY SLSKTLRFEL KPIGKTLENI KKAGLLEQDQ | (SEQ ID |
| type V CRISPR- | HRADSYKKVK KIIDEYHKAF IEKSLSNFEL KYQSEDKLDS LEEYLMYYSM | NO: 145) |
| associated protein | KRIEKTEKDK FAKIQDNLRK QIADHLKGDE SYKTIFSKDL IRKNLPDFVK | |
| Cpf1 [Prevotella | SDEERTLIKE FKDFTTYFKG FYENRENMYS AEDKSTAISH RIIHENLPKF | |
| bryantii B14] | VDNINAFSKI ILIPELREKL NQIYQDFEEY LNVESIDEIF HLDYFSMVMT | |
| | QKQIEVYNAI IGGKSTNDKK IQGLNEYINL YNQKHKDCKL PKLKLLFKQI | |
| | LSDRIAISWL PDNFKDDQEA LDSIDTCYKN LLNDGNVLGE GNLKLLLENI | |
| | DTYNLKGIFI RNDLQLTDIS QKMYASWNVI QDAVILDLKK QVSRKKKESA | |
| | EDYNDRLKKL YTSQESFSIQ YLNDCLRAYG KTENIQDYFA KLGAVNNEHE | |
| | QTINLFAQVR NAYTSVQAIL TTPYPENANL AQDKETVALI KNLLDSLKRL | |
| | QRFIKPLLGK GDESDKDERF YGDFTPLWET LNQITPLYNM VRNYMTRKPY | |
| | SQEKIKLNFE NSTLLGGWDL NKEHDNTAII LRKNGLYYLA IMKKSANKIF | |
| | DKDKLDNSGD CYEKMVYKLL PGANKMLPKV FFSKSRIDEF KPSENIIENY | |
| | KKGTHKKGAN FNLADCHNLI DFFKSSISKH EDWSKFNFHF SDTSSYEDLS | |
| | DFYREVEQQG YSISFCDVSV EYINKMVEKG DLYLFQIYNK DFSEFSKGTP | |
| | NMHTLYWNSL FSKENLNNII YKLNGQAEIF FRKKSLNYKR PTHPAHQAIK | |
| | NKNKCNEKKE SIFDYDLVKD KRYTVDKFQF HVPITMNFKS TGNTNINQQV | |
| | IDYLRTEDDT HIIGIDRGER HLLYLVVIDS HGKIVEQFTL NEIVNEYGGN | |
| | IYRTNYHDLL DTREQNREKA RESWQTIENI KELKEGYISQ VIHKITDLMQ | |
| | KYHAVVVLED LNMGFMRGRQ KVEKQVYQKF EEMLINKLNY LVNKKADQNS | |
| | AGGLLHAYQL TSKFESFQKL GKQSGFLFYI PAWNTSKIDP VTGFVNLFDT | |
| | RYESIDKAKA FFGKFDSIRY NADKDWFEFA FDYNNFTTKA EGTRTNWTIC | |
| | TYGSRIRTFR NQAKNSQWDN EEIDLTKAYK AFFAKHGINI YDNIKEAIAM | |
| | ETEKSFFEDL LHLLKLTLQM RNSITGTTTD YLISPVHDSK GNFYDSRICD | |
| | NSLPANADAN GAYNIARKGL MLIQQIKDST SSNRFKFSPI TNKDWLIFAQ | |
| | EKPYLND | |
| WP_024988992 | MNIKNFTGLY PLSKTLRFEL KPIGKTKENI EKNGILTKDE QRAKDYLIVK | (SEQ ID |
| type V CRISPR- | GFIDEYHKQF IKDRLWDFKL PLESEGEKNS LEEYQELYEL TKRNDAQEAD | NO: 146) |
| associated protein | FTEIKDNLRS SITEQLTKSG SAYDRIFKKE FIREDLVNFL EDEKDKNIVK | |
| Cpf1 [Prevotella | QFEDFTTYFT GFYENRKNMY SSEEKSTAIA YRLIHQNLPK FMDNMRSFAK | |
| albensis] | IANSSVSEHF SDIYESWKEY LNVNSIEEIF QLDYFSETLT QPHIEVYNYI | |
| | IGKKVLEDGT EIKGINEYVN LYNQQQKDKS KRLPFLVPLY KQILSDREKL | |
| | SWIAEEFDSD KKMLSAITES YNHLHNVLMG NENESLRNLL LNIKDYNLEK | |
| | INITNDLSLT EISQNLFGRY DVFTNGIKNK LRVLTPRKKK ETDENFEDRI | |
| | NKIFKTQKSF SIAFLNKLPQ PEMEDGKPRN IEDYFITQGA INTKSIQKED | |
| | IFAQIENAYE DAQVFLQIKD TDNKLSQNKT AVEKIKTLLD ALKELQHFIK | |
| | PLLGSGEENE KDELFYGSFL AIWDELDTIT PLYNKVRNWL TRKPYSTEKI | |
| | KLNFDNAQLL GGWDVNKEHD CAGILLRKND SYYLGIINKK TNHIFDTDIT | |
| | PSDGECYDKI DYKLLPGANK MLPKVFFSKS RIKEFEPSEA IINCYKKGTH | |
| | KKGKNFNLTD CHRLINFFKT SIEKHEDWSK FGFKFSDTET YEDISGFYRE | |
| | VEQQGYRLTS HPVSASYIHS LVKEGKLYLF QIWNKDFSQF SKGTPNLHTL | |
| | YWKMLFDKRN LSDVVYKLNG QAEVFYRKSS IEHQNRIIHP AQHPITNKNE | |
| | LNKKHTSTFK YDIIKDRRYT VDKFQFHVPI TINFKATGQN NINPIVQEVI | |
| | RQNGITHIIQ IDRGERHLLY LSLIDLKGNI IKQMTLNEII NEYKGVTYKT | |
| | NYHNLLEKRE KERTEARHSW SSIESIKELK DGYMSQVIHK ITDMMVKYNA | |
| | IVVLEDLNGG FMRGRQKVEK QVYQKFEKKL IDKLNYLVDK KLDANEVGGV | |
| | LNAYQLTNKF ESFKKIGKQS GFLFYIPAWN TSKIDPITGF VNLFNTRYES | |
| | IKETKVFWSK FDIIRYNKEK NWFEFVFDYN TFTTKAEGTR TKWTLCTHGT | |
| | RIQTFRNPEK NAQWDNKEIN LTESFKALFE KYKIDITSNL KESIMQETEK | |
| | KFFQELHNLL HLTLQMRNSV TGTDIDYLIS PVADEDGNFY DSRINGKNFP | |
| | ENADANGAYN IARKGLMLIR QIKQADPQKK FKFETITNKD WLKFAQDKPY | |
| | LKD | |
| WP_039658684.1 | MQTLFENFTN QYPVSKTLRF ELIPQGKTKD FIEQKGLLKK DEDRAEKYKK | (SEQ ID |
| type V CRISPR- | VKNIIDEYHK DFIEKSLNGL KLDGLEKYKT LYLKQEKDDK DKKAFDKEKE | NO: 147) |
| associated protein | NLRKQIANAF RNNEKFKTLF AKELIKNDLM SFACEEDKKN VKEFEAFTTY | |
| Cpf1 [Smithella sp. | FTGFHQNRAN MYVADEKRTA IASRLIHENL PKFIDNIKIF EKMKKEAPEL | |
| SC_K08D17] | LSPFNQTLKD MKDVIKGTTL EEIFSLDYFN KTLTQSGIDI YNSVIGGRTP | |
| | EEGKTKIKGL NEYINTDFNQ KQTDKKKRQP KFKQLYKQIL SDRQSLSFIA | |
| | EAFKNDTEIL EAIEKFYVNE LLHFSNEGKS TNVLDAIKNA VSNLESFNLT | |
| | KMYFRSGASL TDVSRKVFGE WSIINRALDN YYATTYPIKP REKSEKYEER | |
| | KEKWLKQDFN VSLIQTAIDE YDNETVKGKN SGKVIADYFA KFCDDKETDL | |
| | IQKVNEGYIA VKDLLNTPCP ENEKLGSNKD QVKQIKAFMD SIMDIMHFVR | |
| | PLSLKDTDKE KDETFYSLFT PLYDHLTQTI ALYNKVRNYL TQKPYSTEKI | |
| | KLNFENSTLL GGWDLNKETD NTAIILRKDN LYYLGIMDKR HNRIFRNVPK | |
| | ADKKDFCYEK MVYKLLPGAN KMLPKVFFSQ SRIQEFTPSA KLLENYANET | |
| | HKKGDNFNLN HCHKLIDFFK DSINKHEDWK NFDFRFSATS TYADLSGFYH | |
| | EVEHQGYKIS FQSVADSFID DLVNEGKLYL FQIYNKDFSP FSKGKPNLHT | |
| | LYWKMLFDEN NLKDVVYKLN GEAEVFYRKK SIAEKNTTIH KANESIINKN | |
| | PDNPKATSTF NYDIVKDKRY TIDKFQFHIP ITMNFKAEGI FNMNQRVNQF | |
| | LKANPDINII GIDRGERHLL YYALINQKGK ILKQDTLNVI ANEKQKVDYH | |
| | NLLDKKEGDR ATARQEWGVI ETIKELKEGY LSQVIHKLTD LMIENNAIIV | |
| | MEDLNFGFKR GRQKVEKQVY QKFEKMLIDK LNYLVDKNKK ANELGGLLNA | |
| | FQLANKFESF QKMGKQNGFI FYVPAWNTSK TDPATGFIDF LKPRYENLNQ | |
| | AKDFFEKFDS IRLNSKADYF EFAFDFKNFT EKADGGRTKW TVCTTNEDRY | |
| | AWNRALNNNR GSQEKYDITA ELKSLFDGKV DYKSGKDLKQ QIASQESADF | |
| | FKALMKNLSI TLSLRHNNGE KGDNEQDYIL SPVADSKGRF FDSRKADDDM | |
| | PKNADANGAY HIALKGLWCL EQISKTDDLK KVKLAISNKE WLEFVQTLKG | |
| WP_037385181 | MQTLFENFTN QYPVSKTLRF ELIPQGKTKD FIEQKGLLKK DEDRAEKYKK | (SEQ ID |
| type V CRISPR- | VKNIIDEYHK DFIEKSLNGL KLDGLEEYKT LYLKQEKDDK DKKAFDKEKE | NO: 148) |
| associated protein | NLRKQIANAF RNNEKFKTLF AKELIKNDLM SFACEEDKKN VKEFEAFTTY | |
| Cpf1 [Smithella sp. | FTGFHQNRAN MYVADEKRTA IASRLIHENL PKFIDNIKIF EKMKKEAPEL | |
| SCADC] | LSPFNQTLKD MKDVIKGTTL EEIFSLDYFN KTLTQSGIDI YNSVIGGRTP | |
| | EEGKTKIKGL NEYINTDFNQ KQTDKKKRQP KFKQLYKQIL SDRQSLSFIA | |
| | EAFKNDTEIL EAIEKFYVNE LLHFSNEGKS TNVLDAIKNA VSNLESFNLT | |
| | KIYFRSGTSL TDVSRKVFGE WSIINRALDN YYATTYPIKP REKSEKYEER | |
| | KEKWLKQDFN VSLIQTAIDE YDNETVKGKN SGKVIVDYFA KFCDDKETDL | |
| | IQKVNEGYIA VKDLLNTPYP ENEKLGSNKD QVKQIKAFMD SIMDIMHFVR | |
| | PLSLKDTDKE KDETFYSLFT PLYDHLTQTI ALYNKVRNYL TQKPYSTEKI | |
| | KLNFENSTLL GGWDLNKETD NTAIILRKEN LYYLGIMDKR HNRIFRNVPK | |
| | ADKKDSCYEK MVYKLLPGAN KMLPKVFFSQ SRIQEFTPSA KLLENYENET | |
| | HKKGDNFNLN HCHQLIDFFK DSINKHEDWK NFDFRFSATS TYADLSGFYH | |
| | EVEHQGYKIS FQSIADSFID DLVNEGKLYL FQIYNKDFSP FSKGKPNLHT | |
| | LYWKMLFDEN NLKDVVYKLN GEAEVFYRKK SIAEKNTTIH KANESIINKN | |
| | PDNPKATSTF NYDIVKDKRY TIDKFQFHVP ITMNFKAEGI FNMNQRVNQF | |
| | LKANPDINII GIDRGERHLL YYTLINQKGK ILKQDTLNVI ANEKQKVDYH | |
| | NLLDKKEGDR ATARQEWGVI ETIKELKEGY LSQVIHKLTD LMIENNAIIV | |
| | MEDLNFGFKR GRQKVEKQVY QKFEKMLIDK LNYLVDKNKK ANELGGLLNA | |
| | FQLANKFESF QKMGKQNGFI FYVPAWNTSK TDPATGFIDF LKPRYENLKQ | |
| | AKDFFEKFDS IRLNSKADYF EFAFDFKNFT GKADGGRTKW TVCTTNEDRY | |
| | AWNRALNNNR GSQEKYDITA ELKSLFDGKV DYKSGKDLKQ QIASQELADF | |
| | FRTLMKYLSV TLSLRHNNGE KGETEQDYIL SPVADSMGKF FDSRKAGDDM | |
| | PKNADANGAY HIALKGLWCL EQISKTDDLK KVKLAISNKE WLEFMQTLKG | |
| WP_039871282.1 | MKFTDFTGLY SLSKTLRFEL KPIGKTLENI KKAGLLEQDQ HRADSYKKVK | (SEQ ID |
| type V CRISPR- | KIIDEYHKAF IEKSLSNFEL KYQSEDKLDS LEEYLMYYSM KRIEKTEKDK | NO: 149) |
| associated protein | FAKIQDNLRK QIADHLKGDE SYKTIFSKDL IRKNLPDFVK SDEERTLIKE | |
| Cpf1 [Prevotella | FKDFTTYFKG FYENRENMYS AEDKSTAISH RIIHENLPKF VDNINAFSKI | |
| bryantii B14] | ILIPELREKL NQIYQDFEEY LNVESIDEIF HLDYFSMVMT QKQIEVYNAI | |
| | IGGKSTNDKK IQGLNEYINL YNQKHKDCKL PKLKLLFKQI LSDRIAISWL | |
| | PDNFKDDQEA LDSIDTCYKN LLNDGNVLGE GNLKLLLENI DTYNLKGIFI | |
| | RNDLQLTDIS QKMYASWNVI QDAVILDLKK QVSRKKKESA EDYNDRLKKL | |
| | YTSQESFSIQ YLNDCLRAYG KTENIQDYFA KLGAVNNEHE QTINLFAQVR | |
| | NAYTSVQAIL TTPYPENANL AQDKETVALI KNLLDSLKRL QRFIKPLLGK | |
| | GDESDKDERF YGDFTPLWET LNQITPLYNM VRNYMTRKPY SQEKIKLNFE | |
| | NSTLLGGWDL NKEHDNTAII LRKNGLYYLA IMKKSANKIF DKDKLDNSGD | |
| | CYEKMVYKLL PGANKMLPKV FFSKSRIDEF KPSENIIENY KKGTHKKGAN | |
| | FNLADCHNLI DFFKSSISKH EDWSKFNFHF SDTSSYEDLS DFYREVEQQG | |
| | YSISFCDVSV EYINKMVEKG DLYLFQIYNK DFSEFSKGTP NMHTLYWNSL | |
| | FSKENLNNII YKLNGQAEIF FRKKSLNYKR PTHPAHQAIK NKNKCNEKKE | |
| | SIFDYDLVKD KRYTVDKFQF HVPITMNFKS TGNTNINQQV IDYLRTEDDT | |
| | HIIGIDRGER HLLYLVVIDS HGKIVEQFTL NEIVNEYGGN IYRTNYHDLL | |
| | DTREQNREKA RESWQTIENI KELKEGYISQ VIHKITDLMQ KYHAVVVLED | |
| | LNMGFMRGRQ KVEKQVYQKF EEMLINKLNY LVNKKADQNS AGGLLHAYQL | |
| | TSKFESFQKL GKQSGFLFYI PAWNTSKIDP VTGFVNLFDT RYESIDKAKA | |
| | FFGKFDSIRY NADKDWFEFA FDYNNFTTKA EGTRTNWTIC TYGSRIRTFR | |
| | NQAKNSQWDN EEIDLTKAYK AFFAKHGINI YDNIKEAIAM ETEKSFFEDL | |
| | LHLLKLTLQM RNSITGTTTD YLISPVHDSK GNFYDSRICD NSLPANADAN | |
| | GAYNIARKGL MLIQQIKDST SSNRFKFSPI TNKDWLIFAQ EKPYLND | |
| EKE28449.1 | MFKGDAFTGL YEVQKTLRFE LVPIGLTQSY LENDWVIQKD KEVEENYGKI | (SEQ ID |
| hypothetical protein | KAYFDLIHKE FVRQSLENAW LCQLDDFYEK YIELHNSLET RKDKNLAKQF | NO: 150) |
| ACD 3C00058G0015 | EKVMKSLKKE FVSFFDAKWN EWKQKFSFLK KWWIDVLNEK EVLDLMAEFY | |
| [uncultured | PDEKELFDKF DKFFTYFSNF KESRKNFYAD DGRAWAIATR AIDENLITFI | |
| bacterium (gcode | KNIEDFKKLN SSFREFVNDN FSEEDKQIFE IDFYNNCLLQ PWIDKYNKIV | |
| 4)] | WWYSLENWEK VQWLNEKINN FKQNQNKSNS KDLKFPRMKL LYKQILGDKE | |
| | KKVYIDEIRD DKNLIDLIDN SKRRNQIKID NANDIINDFI NNNAKFELDK | |
| | IYLTRQSINT ISSKYFSSWD YIRWYFWTGE LQEFVSFYDL KETFWKIEYE | |
| | TLENIFKDCY VKGINTESQN NIVFETQGIY ENFLNIFKFE FNQNISQISL | |
| | LEWELDKIQN EDIKKNEKQV EVIKNYFDSV MSVYKMTKYF SLEKWKKRVE | |
| | LDTDNNFYND FNEYLEGFEI WKDYNLVRNY ITKKQVNTDK IKLNFDNSQF | |
| | LTWWDKDKEN ERLGIILRRE WKYYLWILKK WNTLNFGDYL QKEWEIFYEK | |
| | MNYKQLNNVY RQLPRLLFPL TKKLNELKWD ELKKYLSKYI QNFWYNEEIA | |
| | QIKIEFDIFQ ESKEKWEKFD IDKLRKLIEY YKKWVLALYS DLYDLEFIKY | |
| | KNYDDLSIFY SDVEKKMYNL NFTKIDKSLI DGKVKSWELY LFQIYNKDFS | |
| | ESKKEWSTEN IHTKYFKLLF NEKNLQNLVV KLSWWADIFF RDKTENLKFK | |
| | KDKNGQEILD HRRFSQDKIM FHISITLNAN CWDKYWFNQY VNEYMNKERD | |
| | IKIIWIDRWE KHLAYYCVID KSWKIFNNEI WTLNELNWVN YLEKLEKIES | |
| | SRKDSRISWW EIENIKELKN GYISQVINKL TELIVKYNAI IVFEDLNIWF | |
| | KRWRQKIEKQ IYQKLELALA KKLNYLTQKD KKDDEILWNL KALQLVPKVN | |
| | DYQDIWNYKQ SWIMFYVRAN YTSVTCPNCW LRKNLYISNS ATKENQKKSL | |
| | NSIAIKYNDW KFSFSYEIDD KSWKQKQSLN KKKFIVYSDI ERFVYSPLEK | |
| | LTKVIDVNKK LLELFRDFNL SLDINKQIQE KDLDSVFFKS LTHLFNLILQ | |
| | LRNSDSKDNK DYISCPSCYY HSNNWLQWFE FNWDANWAYN IARKGIILLD | |
| | RIRKNQEKPD LYVSDIDWDN FVQSNQFPNT IIPIQNIEKQ VPLNIKI | |
| WP_018359861.1 | MKTQHFFEDF TSLYSLSKTI RFELKPIGKT LENIKKNGLI RRDEQRLDDY | (SEQ ID |
| type V CRISPR- | EKLKKVIDEY HEDFIANILS SFSFSEEILQ SYIQNLSESE ARAKIEKTMR | NO: 151) |
| associated protein | DTLAKAFSED ERYKSIFKKE LVKKDIPVWC PAYKSLCKKF DNFTTSLVPF | |
| Cpf] | HENRKNLYTS NEITASIPYR IVHVNLPKFI QNIEALCELQ KKMGADLYLE | |
| [Porphyromonas | MMENLRNVWP SFVKTPDDLC NLKTYNHLMV QSSISEYNRF VGGYSTEDGT | |
| macacae] | KHQGINEWIN IYRQRNKEMR LPGLVFLHKQ ILAKVDSSSF ISDTLENDDQ | |
| | VFCVLRQFRK LFWNTVSSKE DDAASLKDLF CGLSGYDPEA IYVSDAHLAT | |
| | ISKNIFDRWN YISDAIRRKT EVLMPRKKES VERYAEKISK QIKKRQSYSL | |
| | AELDDLLAHY SEESLPAGFS LLSYFTSLGG QKYLVSDGEV ILYEEGSNIW | |
| | DEVLIAFRDL QVILDKDFTE KKLGKDEEAV SVIKKALDSA LRLRKFFDLL | |
| | SGTGAEIRRD SSFYALYTDR MDKLKGLLKM YDKVRNYLTK KPYSIEKFKL | |
| | HFDNPSLLSG WDKNKELNNL SVIFRQNGYY YLGIMTPKGK NLFKTLPKLG | |
| | AEEMFYEKME YKQIAEPMLM LPKVFFPKKT KPAFAPDQSV VDIYNKKTFK | |
| | TGQKGFNKKD LYRLIDFYKE ALTVHEWKLF NFSFSPTEQY RNIGEFFDEV | |
| | REQAYKVSMV NVPASYIDEA VENGKLYLFQ IYNKDFSPYS KGIPNLHTLY | |
| | WKALFSEQNQ SRVYKLCGGG ELFYRKASLH MQDTTVHPKG ISIHKKNLNK | |
| | KGETSLFNYD LVKDKRFTED KFFFHVPISI NYKNKKITNV NQMVRDYIAQ | |
| | NDDLQIIGID RGERNLLYIS RIDTRGNLLE QFSLNVIESD KGDLRTDYQK | |
| | ILGDREQERL RRRQEWKSIE SIKDLKDGYM SQVVHKICNM VVEHKAIVVL | |
| | ENLNLSFMKG RKKVEKSVYE KFERMLVDKL NYLVVDKKNL SNEPGGLYAA | |
| | YQLTNPLFSF EELHRYPQSG ILFFVDPWNT SLTDPSTGFV NLLGRINYTN | |
| | VGDARKFFDR FNAIRYDGKG NILFDLDLSR FDVRVETQRK LWTLTTFGSR | |
| | IAKSKKSGKW MVERIENLSL CFLELFEQFN IGYRVEKDLK KAILSQDRKE | |
| | FYVRLIYLFN LMMQIRNSDG EEDYILSPAL NEKNLQFDSR LIEAKDLPVD | |
| | ADANGAYNVA RKGLMVVQRI KRGDHESIHR IGRAQWLRYV QEGIVE | |
| WP_013282991 | MLLYENYTKR NQITKSLRLE LRPQGKTLRN IKELNLLEQD KAIYALLERL | (SEQ ID |
| type V CRISPR- | KPVIDEGIKD IARDTLKNCE LSFEKLYEHF LSGDKKAYAK ESERLKKEIV | NO: 152) |
| associated protein | KTLIKNLPEG IGKISEINSA KYLNGVLYDF IDKTHKDSEE KQNILSDILE | |
| Cpf1 [Butyrivibrio | TKGYLALFSK FLTSRITTLE QSMPKRVIEN FEIYAANIPK MQDALERGAV | |
| proteoclasticus] | SFAIEYESIC SVDYYNQILS QEDIDSYNRL ISGIMDEDGA KEKGINQTIS | |
| | EKNIKIKSEH LEEKPFRILK QLHKQILEER EKAFTIDHID SDEEVVQVTK | |
| | EAFEQTKEQW ENIKKINGFY AKDPGDITLF IVVGPNQTHV LSQLIYGEHD | |
| | RIRLLLEEYE KNTLEVLPRR TKSEKARYDK FVNAVPKKVA KESHTFDGLQ | |
| | KMTGDDRLFI LYRDELARNY MRIKEAYGTF ERDILKSRRG IKGNRDVQES | |
| | LVSFYDELTK FRSALRIINS GNDEKADPIF YNTFDGIFEK ANRTYKAENL | |
| | CRNYVTKSPA DDARIMASCL GTPARLRTHW WNGEENFAIN DVAMIRRGDE | |
| | YYYFVLTPDV KPVDLKTKDE TDAQIFVQRK GAKSFLGLPK ALFKCILEPY | |
| | FESPEHKNDK NCVIEEYVSK PLTIDRRAYD IFKNGTFKKT NIGIDGLTEE | |
| | KFKDDCRYLI DVYKEFIAVY TRYSCFNMSG LKRADEYNDI GEFFSDVDTR | |
| | LCTMEWIPVS FERINDMVDK KEGLLFLVRS MFLYNRPRKP YERTFIQLFS | |
| | DSNMEHTSML LNSRAMIQYR AASLPRRVTH KKGSILVALR DSNGEHIPMH | |
| | IREAIYKMKN NFDISSEDFI MAKAYLAEHD VAIKKANEDI IRNRRYTEDK | |
| | FFLSLSYTKN ADISARTLDY INDKVEEDTQ DSRMAVIVTR NLKDLTYVAV | |
| | VDEKNNVLEE KSLNEIDGVN YRELLKERTK IKYHDKTRLW QYDVSSKGLK | |
| | EAYVELAVTQ ISKLATKYNA VVVVESMSST FKDKFSFLDE QIFKAFEARL | |
| | CARMSDLSFN TIKEGEAGSI SNPIQVSNNN GNSYQDGVIY FLNNAYTRTL | |
| | CPDTGFVDVF DKTRLITMQS KRQFFAKMKD IRIDDGEMLF TFNLEEYPTK | |
| | RLLDRKEWTV KIAGDGSYFD KDKGEYVYVN DIVREQIIPA LLEDKAVFDG | |
| | NMAEKFLDKT AISGKSVELI YKWFANALYG IITKKDGEKI YRSPITGTEI | |
| | DVSKNTTYNF GKKFMFKQEY RGDGDFLDAF LNYMQAQDIA V | |
| WP_048112740.1 | MNNYDEFTKL YPIQKTIRFE LKPQGRTMEH LETFNFFEED RDRAEKYKIL | (SEQ ID |
| type V CRISPR- | KEAIDEYHKK FIDEHLTNMS LDWNSLKQIS EKYYKSREEK DKKVFLSEQK | NO: 153) |
| associated protein | RMRQEIVSEF KKDDRFKDLF SKKLFSELLK EEIYKKGNHQ EIDALKSFDK | |
| Cpf1 [Candidatus | FSGYFIGLHE NRKNMYSDGD EITAISNRIV NENFPKFLDN LQKYQEARKK | |
| Methanoplasma termitum] | YPEWIIKAES ALVAHNIKMD EVFSLEYFNK VLNQEGIQRY NLALGGYVTK | |
| | SGEKMMGLND ALNLAHQSEK SSKGRIHMTP LFKQILSEKE SFSYIPDVFT | |
| | EDSQLLPSIG GFFAQIENDK DGNIFDRALE LISSYAEYDT ERIYIRQADI | |
| | NRVSNVIFGE WGTLGGLMRE YKADSINDIN LERTCKKVDK WLDSKEFALS | |
| | DVLEAIKRTG NNDAFNEYIS KMRTAREKID AARKEMKFIS EKISGDEESI | |
| | HIIKTLLDSV QQFLHFFNLF KARQDIPLDG AFYAEFDEVH SKLFAIVPLY | |
| | NKVRNYLTKN NLNTKKIKLN FKNPTLANGW DQNKVYDYAS LIFLRDGNYY | |
| | LGIINPKRKK NIKFEQGSGN GPFYRKMVYK QIPGPNKNLP RVFLTSTKGK | |
| | KEYKPSKEII EGYEADKHIR GDKFDLDFCH KLIDFFKESI EKHKDWSKFN | |
| | FYFSPTESYG DISEFYLDVE KQGYRMHFEN ISAETIDEYV EKGDLFLFQI | |
| | YNKDFVKAAT GKKDMHTIYW NAAFSPENLQ DVVVKLNGEA ELFYRDKSDI | |
| | KEIVHREGEI LVNRTYNGRT PVPDKIHKKL TDYHNGRTKD LGEAKEYLDK | |
| | VRYFKAHYDI TKDRRYLNDK IYFHVPLTLN FKANGKKNLN KMVIEKFLSD | |
| | EKAHIIGIDR GERNLLYYSI IDRSGKIIDQ QSLNVIDGFD YREKLNQREI | |
| | EMKDARQSWN AIGKIKDLKE GYLSKAVHEI TKMAIQYNAI VVMEELNYGF | |
| | KRGRFKVEKQ IYQKFENMLI DKMNYLVFKD APDESPGGVL NAYQLTNPLE | |
| | SFAKLGKQTG ILFYVPAAYT SKIDPTTGFV NLFNTSSKTN AQERKEFLQK | |
| | FESISYSAKD GGIFAFAFDY RKFGTSKTDH KNVWTAYTNG ERMRYIKEKK | |
| | RNELFDPSKE IKEALTSSGI KYDGGQNILP DILRSNNNGL IYTMYSSFIA | |
| | AIQMRVYDGK EDYIISPIKN SKGEFFRTDP KRRELPIDAD ANGAYNIALR | |
| | GELTMRAIAE KFDPDSEKMA KLELKHKDWF EFMQTRGD | |
| WP_027407524.1 | MVAFIDEFVG QYPVSKTLRF EARPVPETKK WLESDQCSVL FNDQKRNEYY | (SEQ ID |
| type V CRISPR- | GVLKELLDDY YRAYIEDALT SFTLDKALLE NAYDLYCNRD TNAFSSCCEK | NO: 154) |
| associated protein | LRKDLVKAFG NLKDYLLGSD QLKDLVKLKA KVDAPAGKGK KKIEVDSRLI | |
| Cpf1 [Anaerovibrio | NWLNNNAKYS AEDREKYIKA IESFEGFVTY LTNYKQAREN MFSSEDKSTA | |
| sp. RM50] | IAFRVIDQNM VTYFGNIRIY EKIKAKYPEL YSALKGFEKF FSPTAYSEIL | |
| | SQSKIDEYNY QCIGRPIDDA DFKGVNSLIN EYRQKNGIKA RELPVMSMLY | |
| | KQILSDRDNS FMSEVINRNE EAIECAKNGY KVSYALFNEL LQLYKKIFTE | |
| | DNYGNIYVKT QPLTELSQAL FGDWSILRNA LDNGKYDKDI INLAELEKYF | |
| | SEYCKVLDAD DAAKIQDKFN LKDYFIQKNA LDATLPDLDK ITQYKPHLDA | |
| | MLQAIRKYKL FSMYNGRKKM DVPENGIDFS NEFNAIYDKL SEFSILYDRI | |
| | RNFATKKPYS DEKMKLSFNM PTMLAGWDYN NETANGCFLF IKDGKYFLGV | |
| | ADSKSKNIFD FKKNPHLLDK YSSKDIYYKV KYKQVSGSAK MLPKVVFAGS | |
| | NEKIFGHLIS KRILEIREKK LYTAAAGDRK AVAEWIDFMK SAIAIHPEWN | |
| | LFQLYTKDFS DKKKKKGTDN EDIDKQTYSL EKVEIPTEYI DEMVSQHKLY | |
| | EYFKFKFKNT AEYDNANKFY LHTMYWHGVF SDENLKAVTE GTQPIIKLNG | |
| | EAEMFMRNPS IEFQVTHEHN KPIANKNPLN TKKESVFNYD LIKDKRYTER | |
| | KFYFHCPITL NFRADKPIKY NEKINRFVEN NPDVCIIGID RGERHLLYYT | |
| | VINQTGDILE QGSLNKISGS YTNDKGEKVN KETDYHDLLD RKEKGKHVAQ | |
| | QAWETIENIK ELKAGYLSQV VYKLTQLMLQ YNAVIVLENL NVGFKRGRTK | |
| | VEKQVYQKFE KAMIDKLNYL VFKDRGYEMN GSYAKGLQLT DKFESFDKIG | |
| | KQTGCIYYVI PSYTSHIDPK TGFVNLLNAK LRYENITKAQ DTIRKFDSIS | |
| | YNAKADYFEF AFDYRSFGVD MARNEWVVCT CGDLRWEYSA KTRETKAYSV | |
| | TDRLKELFKA HGIDYVGGEN LVSHITEVAD KHFLSTLLFY LRLVLKMRYT | |
| | VSGTENENDF ILSPVEYAPG KFFDSREATS TEPMNADANG AYHIALKGLM | |
| | TIRGIEDGKL HNYGKGGENA AWFKFMQNQE YKNNG | |
| WP_044910712.1 | MDYGNGQFER RAPLTKTITL RLKPIGETRE TIREQKLLEQ DAAFRKLVET | (SEQ ID |
| type V CRISPR- | VTPIVDDCIR KIADNALCHF GTEYDFSCLG NAISKNDSKA IKKETEKVEK | NO: 155) |
| associated protein | LLAKVLTENL PDGLRKVNDI NSAAFIQDTL TSFVQDDADK RVLIQELKGK | |
| Cpf1 | TVLMQRFLTT RITALTVWLP DRVFENFNIF IENAEKMRIL LDSPLNEKIM | |
| [Lachnospiraceae | KFDPDAEQYA SLEFYGQCLS QKDIDSYNLI ISGIYADDEV KNPGINEIVK | |
| bacterium MC2017] | EYNQQIRGDK DESPLPKLKK LHKQILMPVE KAFFVRVLSN DSDARSILEK | |
| | ILKDTEMLPS KIIEAMKEAD AGDIAVYGSR LHELSHVIYG DHGKLSQIIY | |
| | DKESKRISEL METLSPKERK ESKKRLEGLE EHIRKSTYTF DELNRYAEKN | |
| | VMAAYIAAVE ESCAEIMRKE KDLRTLLSKE DVKIRGNRHN TLIVKNYFNA | |
| | WTVFRNLIRI LRRKSEAEID SDFYDVLDDS VEVLSLTYKG ENLCRSYITK | |
| | KIGSDLKPEI ATYGSALRPN SRWWSPGEKF NVKFHTIVRR DGRLYYFILP | |
| | KGAKPVELED MDGDIECLQM RKIPNPTIFL PKLVFKDPEA FFRDNPEADE | |
| | FVFLSGMKAP VTITRETYEA YRYKLYTVGK LRDGEVSEEE YKRALLQVLT | |
| | AYKEFLENRM IYADLNFGFK DLEEYKDSSE FIKQVETHNT FMCWAKVSSS | |
| | QLDDLVKSGN GLLFEIWSER LESYYKYGNE KVLRGYEGVL LSILKDENLV | |
| | SMRTLLNSRP MLVYRPKESS KPMVVHRDGS RVVDRFDKDG KYIPPEVHDE | |
| | LYRFFNNLLI KEKLGEKARK ILDNKKVKVK VLESERVKWS KFYDEQFAVT | |
| | FSVKKNADCL DTTKDLNAEV MEQYSESNRL ILIRNTTDIL YYLVLDKNGK | |
| | VLKQRSLNII NDGARDVDWK ERFRQVTKDR NEGYNEWDYS RTSNDLKEVY | |
| | LNYALKEIAE AVIEYNAILI IEKMSNAFKD KYSFLDDVTF KGFETKLLAK | |
| | LSDLHFRGIK DGEPCSFTNP LQLCQNDSNK ILQDGVIFMV PNSMTRSLDP | |
| | DTGFIFAIND HNIRTKKAKL NFLSKFDQLK VSSEGCLIMK YSGDSLPTHN | |
| | TDNRVWNCCC NHPITNYDRE TKKVEFIEEP VEELSRVLEE NGIETDTELN | |
| | KLNERENVPG KVVDAIYSLV LNYLRGTVSG VAGQRAVYYS PVTGKKYDIS | |
| | FIQAMNLNRK CDYYRIGSKE RGEWTDFVAQ LIN | |
| WP_081834226 | MTMDYGNGQF ERRAPLTKTI TLRLKPIGET RETIREQKLL EQDAAFRKLV | (SEQ ID |
| type V CRISPR- | ETVTPIVDDC IRKIADNALC HFGTEYDFSC LGNAISKNDS KAIKKETEKV | NO: 156) |
| associated protein | EKLLAKVLTE NLPDGLRKVN DINSAAFIQD TLTSFVQDDA DKRVLIQELK | |
| Cpf1 | GKTVLMQRFL TTRITALTVW LPDRVFENFN IFIENAEKMR ILLDSPLNEK | |
| [Lachnospiraceae | IMKFDPDAEQ YASLEFYGQC LSQKDIDSYN LIISGIYADD EVKNPGINEI | |
| bacterium | VKEYNQQIRG DKDESPLPKL KKLHKQILMP VEKAFFVRVL SNDSDARSIL | |
| MC2017]. | EKILKDTEML PSKIIEAMKE ADAGDIAVYG SRLHELSHVI YGDHGKLSQI | |
| | IYDKESKRIS ELMETLSPKE RKESKKRLEG LEEHIRKSTY TFDELNRYAE | |
| | KNVMAAYIAA VEESCAEIMR KEKDLRTLLS KEDVKIRGNR HNTLIVKNYF | |
| | NAWTVFRNLI RILRRKSEAE IDSDFYDVLD DSVEVLSLTY KGENLCRSYI | |
| | TKKIGSDLKP EIATYGSALR PNSRWWSPGE KFNVKFHTIV RRDGRLYYFI | |
| | LPKGAKPVEL EDMDGDIECL QMRKIPNPTI FLPKLVFKDP EAFFRDNPEA | |
| | DEFVFLSGMK APVTITRETY EAYRYKLYTV GKLRDGEVSE EEYKRALLQV | |
| | LTAYKEFLEN RMIYADLNFG FKDLEEYKDS SEFIKQVETH NTFMCWAKVS | |
| | SSQLDDLVKS GNGLLFEIWS ERLESYYKYG NEKVLRGYEG VLLSILKDEN | |
| | LVSMRTLLNS RPMLVYRPKE SSKPMVVHRD GSRVVDRFDK DGKYIPPEVH | |
| | DELYRFFNNL LIKEKLGEKA RKILDNKKVK VKVLESERVK WSKFYDEQFA | |
| | VTFSVKKNAD CLDTTKDLNA EVMEQYSESN RLILIRNTTD ILYYLVLDKN | |
| | GKVLKQRSLN IINDGARDVD WKERFRQVTK DRNEGYNEWD YSRTSNDLKE | |
| | VYLNYALKEI AEAVIEYNAI LIIEKMSNAF KDKYSFLDDV TFKGFETKLL | |
| | AKLSDLHFRG IKDGEPCSFT NPLQLCQNDS NKILQDGVIF MVPNSMTRSL | |
| | DPDTGFIFAI NDHNIRTKKA KLNFLSKFDQ LKVSSEGCLI MKYSGDSLPT | |
| | HNTDNRVWNC CCNHPITNYD RETKKVEFIE EPVEELSRVL EENGIETDTE | |
| | LNKLNERENV PGKVVDAIYS LVLNYLRGTV SGVAGQRAVY YSPVTGKKYD | |
| | ISFIQAMNLN RKCDYYRIGS KERGEWTDFV AQLIN | |
| WP_027216152.1 | MYYESLTKLY PIKKTIRNEL VPIGKTLENI KKNNILEADE DRKIAYIRVK | (SEQ ID |
| type V CRISPR- | AIMDDYHKRL INEALSGFAL IDLDKAANLY LSRSKSADDI ESFSRFQDKL | NO: 157) |
| associated protein | RKAIAKRLRE HENFGKIGNK DIIPLLQKLS ENEDDYNALE SFKNFYTYFE | |
| Cpf1 [Butyrivibrio | SYNDVRLNLY SDKEKSSTVA YRLINENLPR FLDNIRAYDA VQKAGITSEE | |
| fibrisolvens] | LSSEAQDGLF LVNTFNNVLI QDGINTYNED IGKLNVAINL YNQKNASVQG | |
| | FRKVPKMKVL YKQILSDREE SFIDEFESDT ELLDSLESHY ANLAKYFGSN | |
| | KVQLLFTALR ESKGVNVYVK NDIAKTSFSN VVFGSWSRID ELINGEYDDN | |
| | NNRKKDEKYY DKRQKELKKN KSYTIEKIIT LSTEDVDVIG KYIEKLESDI | |
| | DDIRFKGKNF YEAVLCGHDR SKKLSKNKGA VEAIKGYLDS VKDFERDLKL | |
| | INGSGQELEK NLVVYGEQEA VLSELSGIDS LYNMTRNYLT KKPFSTEKIK | |
| | LNFNKPTFLD GWDYGNEEAY LGFFMIKEGN YFLAVMDANW NKEFRNIPSV | |
| | DKSDCYKKVI YKQISSPEKS IQNLMVIDGK TVKKNGRKEK EGIHSGENLI | |
| | LEELKNTYLP KKINDIRKRR SYLNGDTFSK KDLTEFIGYY KORVIEYYNG | |
| | YSFYFKSDDD YASFKEFQED VGRQAYQISY VDVPVSFVDD LINSGKLYLF | |
| | RVYNKDFSEY SKGRLNLHTL YFKMLFDERN LKNVVYKLNG QAEVFYRPSS | |
| | IKKEELIVHR AGEEIKNKNP KRAAQKPTRR LDYDIVKDRR YSQDKFMLHT | |
| | SIIMNFGAEE NVSFNDIVNG VLRNEDKVNV IGIDRGERNL LYVVVIDPEG | |
| | KILEQRSLNC ITDSNLDIET DYHRLLDEKE SDRKIARRDW TTIENIKELK | |
| | AGYLSQVVHI VAELVLKYNA IICLEDLNFG FKRGRQKVEK QVYQKFEKML | |
| | IDKLNYLVMD KSREQLSPEK ISGALNALQL TPDFKSFKVL GKQTGIIYYV | |
| | PAYLTSKIDP MTGFANLFYV KYENVDKAKE FFSKFDSIKY NKDGKNWNTK | |
| | GYFEFAFDYK KFTDRAYGRV SEWTVCTVGE RIIKFKNKEK NNSYDDKVID | |
| | LTNSLKELFD SYKVTYESEV DLKDAILAID DPAFYRDLTR RLQQTLQMRN | |
| | SSCDGSRDYI ISPVKNSKGE FFCSDNNDDT TPNDADANGA FNIARKGLWV | |
| | LNEIRNSEEG SKINLAMSNA QWLEYAQDNT I | |
| WP_016301126.1 | MHENNGKIAD NFIGIYPVSK TLRFELKPVG KTQEYIEKHG ILDEDLKRAG | (SEQ ID |
| type V CRISPR- | DYKSVKKIID AYHKYFIDEA LNGIQLDGLK NYYELYEKKR DNNEEKEFQK | NO: 158) |
| associated protein | IQMSLRKQIV KRFSEHPQYK YLFKKELIKN VLPEFTKDNA EEQTLVKSFQ | |
| Cpf1 | EFTTYFEGFH QNRKNMYSDE EKSTAIAYRV VHQNLPKYID NMRIFSMILN | |
| [Lachnospiraceae | TDIRSDLTEL FNNLKTKMDI TIVEEYFAID GFNKVVNQKG IDVYNTILGA | |
| bacterium COE1] | FSTDDNTKIK GLNEYINLYN QKNKAKLPKL KPLFKQILSD RDKISFIPEQ | |
| | FDSDTEVLEA VDMFYNRLLQ FVIENEGQIT ISKLLTNFSA YDLNKIYVKN | |
| | DTTISAISND LFDDWSYISK AVRENYDSEN VDKNKRAAAY EEKKEKALSK | |
| | IKMYSIEELN FFVKKYSCNE CHIEGYFERR ILEILDKMRY AYESCKILHD | |
| | KGLINNISLC QDRQAISELK DFLDSIKEVQ WLLKPLMIGQ EQADKEEAFY | |
| | TELLRIWEEL EPITLLYNKV RNYVTKKPYT LEKVKLNFYK STLLDGWDKN | |
| | KEKDNLGIIL LKDGQYYLGI MNRRNNKIAD DAPLAKTDNV YRKMEYKLLT | |
| | KVSANLPRIF LKDKYNPSEE MLEKYEKGTH LKGENFCIDD CRELIDFFKK | |
| | GIKQYEDWGQ FDFKFSDTES YDDISAFYKE VEHQGYKITF RDIDETYIDS | |
| | LVNEGKLYLF QIYNKDFSPY SKGTKNLHTL YWEMLFSQQN LQNIVYKLNG | |
| | NAEIFYRKAS INQKDVVVHK ADLPIKNKDP QNSKKESMFD YDIIKDKRFT | |
| | CDKYQFHVPI TMNFKALGEN HFNRKVNRLI HDAENMHIIG IDRGERNLIY | |
| | LCMIDMKGNI VKQISLNEII SYDKNKLEHK RNYHQLLKTR EDENKSARQS | |
| | WQTIHTIKEL KEGYLSQVIH VITDLMVEYN AIVVLEDLNF GFKQGRQKFE | |
| | RQVYQKFEKM LIDKLNYLVD KSKGMDEDGG LLHAYQLTDE FKSFKQLGKQ | |
| | SGFLYYIPAW NTSKLDPTTG FVNLFYTKYE SVEKSKEFIN NFTSILYNQE | |
| | REYFEFLFDY SAFTSKAEGS RLKWTVCSKG ERVETYRNPK KNNEWDTQKI | |
| | DLTFELKKLF NDYSISLLDG DLREQMGKID KADFYKKFMK LFALIVQMRN | |
| | SDEREDKLIS PVLNKYGAFF ETGKNERMPL DADANGAYNI ARKGLWIIEK | |
| | IKNTDVEQLD KVKLTISNKE WLQYAQEHIL | |
| WP_035635841.1 | MSKLEKFTNC YSLSKTLRFK AIPVGKTQEN IDNKRLLVED EKRAEDYKGV | (SEQ ID |
| type V CRISPR- | KKLLDRYYLS FINDVLHSIK LKNLNNYISL FRKKTRTEKE NKELENLEIN | NO: 159) |
| associated protein | LRKEIAKAFK GNEGYKSLFK KDIIETILPE FLDDKDEIAL VNSFNGFTTA | |
| Cpf1 | FTGFFDNREN MFSEEAKSTS IAFRCINENL TRYISNMDIF EKVDAIFDKH | |
| [Lachnospiraceae | EVQEIKEKIL NSDYDVEDFF EGEFFNFVLT QEGIDVYNAI IGGFVTESGE | |
| bacterium ND2006] | KIKGLNEYIN LYNQKTKQKL PKFKPLYKQV LSDRESLSFY GEGYTSDEEV | |
| | LEVFRNTLNK NSEIFSSIKK LEKLFKNFDE YSSAGIFVKN GPAISTISKD | |
| | IFGEWNVIRD KWNAEYDDIH LKKKAVVTEK YEDDRRKSFK KIGSFSLEQL | |
| | QEYADADLSV VEKLKEIIIQ KVDEIYKVYG SSEKLFDADF VLEKSLKKND | |
| | AVVAIMKDLL DSVKSFENYI KAFFGEGKET NRDESFYGDF VLAYDILLKV | |
| | DHIYDAIRNY VTQKPYSKDK FKLYFQNPQF MGGWDKDKET DYRATILRYG | |
| | SKYYLAIMDK KYAKCLQKID KDDVNGNYEK INYKLLPGPN KMLPKVFFSK | |
| | KWMAYYNPSE DIQKIYKNGT FKKGDMFNLN DCHKLIDFFK DSISRYPKWS | |
| | NAYDFNFSET EKYKDIAGFY REVEEQGYKV SFESASKKEV DKLVEEGKLY | |
| | MFQIYNKDFS DKSHGTPNLH TMYFKLLFDE NNHGQIRLSG GAELFMRRAS | |
| | LKKEELVVHP ANSPIANKNP DNPKKTTTLS YDVYKDKRFS EDQYELHIPI | |
| | AINKCPKNIF KINTEVRVLL KHDDNPYVIG IDRGERNLLY IVVVDGKGNI | |
| | VEQYSLNEII NNFNGIRIKT DYHSLLDKKE KERFEARQNW TSIENIKELK | |
| | AGYISQVVHK ICELVEKYDA VIALEDLNSG FKNSRVKVEK QVYQKFEKML | |
| | IDKLNYMVDK KSNPCATGGA LKGYQITNKF ESFKSMSTQN GFIFYIPAWL | |
| | TSKIDPSTGF VNLLKTKYTS IADSKKFISS FDRIMYVPEE DLFEFALDYK | |
| | NFSRTDADYI KKWKLYSYGN RIRIFRNPKK NNVFDWEEVC LTSAYKELFN | |
| | KYGINYQQGD IRALLCEQSD KAFYSSFMAL MSLMLQMRNS ITGRTDVDFL | |
| | ISPVKNSDGI FYDSRNYEAQ ENAILPKNAD ANGAYNIARK VLWAIGQFKK | |
| | AEDEKLDKVK IAISNKEWLE YAQTSVKH | |
| WP_051666128.1 | MLKNVGIDRL DVEKGRKNMS KLEKFTNCYS LSKTLRFKAI PVGKTQENID | (SEQ ID |
| type V CRISPR- | NKRLLVEDEK RAEDYKGVKK LLDRYYLSFI NDVLHSIKLK NLNNYISLFR | NO: 160) |
| associated protein | KKTRTEKENK ELENLEINLR KEIAKAFKGN EGYKSLFKKD IIETILPEFL | |
| Cpf1 | DDKDEIALVN SFNGFTTAFT GFFDNRENMF SEEAKSTSIA FRCINENLTR | |
| [Lachnospiraceae | YISNMDIFEK VDAIFDKHEV QEIKEKILNS DYDVEDFFEG EFFNFVLTQE | |
| bacterium ND2006] | GIDVYNAIIG GFVTESGEKI KGLNEYINLY NQKTKQKLPK FKPLYKQVLS | |
| | DRESLSFYGE GYTSDEEVLE VFRNTLNKNS EIFSSIKKLE KLFKNFDEYS | |
| | SAGIFVKNGP AISTISKDIF GEWNVIRDKW NAEYDDIHLK KKAVVTEKYE | |
| | DDRRKSFKKI GSFSLEQLQE YADADLSVVE KLKEIIIQKV DEIYKVYGSS | |
| | EKLFDADFVL EKSLKKNDAV VAIMKDLLDS VKSFENYIKA FFGEGKETNR | |
| | DESFYGDFVL AYDILLKVDH IYDAIRNYVT QKPYSKDKFK LYFQNPQFMG | |
| | GWDKDKETDY RATILRYGSK YYLAIMDKKY AKCLQKIDKD DVNGNYEKIN | |
| | YKLLPGPNKM LPKVFFSKKW MAYYNPSEDI QKIYKNGTFK KGDMFNLNDC | |
| | HKLIDFFKDS ISRYPKWSNA YDFNFSETEK YKDIAGFYRE VEEQGYKVSF | |
| | ESASKKEVDK LVEEGKLYMF QIYNKDFSDK SHGTPNLHTM YFKLLFDENN | |
| | HGQIRLSGGA ELFMRRASLK KEELVVHPAN SPIANKNPDN PKKTTTLSYD | |
| | VYKDKRFSED QYELHIPIAI NKCPKNIFKI NTEVRVLLKH DDNPYVIGID | |
| | RGERNLLYIV VVDGKGNIVE QYSLNEIINN FNGIRIKTDY HSLLDKKEKE | |
| | RFEARQNWTS IENIKELKAG YISQVVHKIC ELVEKYDAVI ALEDLNSGFK | |
| | NSRVKVEKQV YQKFEKMLID KLNYMVDKKS NPCATGGALK GYQITNKFES | |
| | FKSMSTQNGF IFYIPAWLTS KIDPSTGFVN LLKTKYTSIA DSKKFISSFD | |
| | RIMYVPEEDL FEFALDYKNF SRTDADYIKK WKLYSYGNRI RIFRNPKKNN | |
| | VFDWEEVCLT SAYKELFNKY GINYQQGDIR ALLCEQSDKA FYSSFMALMS | |
| | LMLQMRNSIT GRTDVDFLIS PVKNSDGIFY DSRNYEAQEN AILPKNADAN | |
| | GAYNIARKVL WAIGQFKKAE DEKLDKVKIA ISNKEWLEYA QTSVKH | |
| WP_015504779.1 | MDAKEFTGQY PLSKTLRFEL RPIGRTWDNL EASGYLAEDR HRAECYPRAK | (SEQ ID |
| type V CRISPR- | ELLDDNHRAF LNRVLPQIDM DWHPIAEAFC KVHKNPGNKE LAQDYNLQLS | NO: 161) |
| associated protein | KRRKEISAYL QDADGYKGLF AKPALDEAMK IAKENGNESD IEVLEAFNGF | |
| Cpf1 [Candidatus | SVYFTGYHES RENIYSDEDM VSVAYRITED NFPRFVSNAL IFDKLNESHP | |
| Methanomethylophilus | DIISEVSGNL GVDDIGKYFD VSNYNNFLSQ AGIDDYNHII GGHTTEDGLI | |
| alvus] | QAFNVVLNLR HQKDPGFEKI QFKQLYKQIL SVRTSKSYIP KQFDNSKEMV | |
| | DCICDYVSKI EKSETVERAL KLVRNISSFD LRGIFVNKKN LRILSNKLIG | |
| | DWDAIETALM HSSSSENDKK SVYDSAEAFT LDDIFSSVKK FSDASAEDIG | |
| | NRAEDICRVI SETAPFINDL RAVDLDSIND DGYEAAVSKI RESLEPYMDL | |
| | FHELEIFSVG DEFPKCAAFY SELEEVSEQL IEIIPLFNKA RSFCTRKRYS | |
| | TDKIKVNLKF PTLADGWDLN KERDNKAAIL RKDGKYYLAI LDMKKDLSSI | |
| | RTSDEDESSF EKMEYKLLPS PVKMLPKIFV KSKAAKEKYG LTDRMLECYD | |
| | KGMHKSGSAF DLGFCHELID YYKRCIAEYP GWDVFDFKFR ETSDYGSMKE | |
| | FNEDVAGAGY YMSLRKIPCS EVYRLLDEKS IYLFQIYNKD YSENAHGNKN | |
| | MHTMYWEGLF SPQNLESPVF KLSGGAELFF RKSSIPNDAK TVHPKGSVLV | |
| | PRNDVNGRRI PDSTYRELTR YFNRGDCRIS DEAKSYLDKV KTKKADHDIV | |
| | KDRRFTVDKM MFHVPIAMNF KAISKPNLNK KVIDGIIDDQ DLKIIGIDRG | |
| | ERNLIYVTMV DRKGNILYQD SLNILNGYDY RKALDVREYD NKEARRNWTK | |
| | VEGIRKMKEG YLSLAVSKLA DMIIENNAII VMEDLNHGFK AGRSKIEKQV | |
| | YQKFESMLIN KLGYMVLKDK SIDQSGGALH GYQLANHVTT LASVGKQCGV | |
| | IFYIPAAFTS KIDPTTGFAD LFALSNVKNV ASMREFFSKM KSVIYDKAEG | |
| | KFAFTFDYLD YNVKSECGRT LWTVYTVGER FTYSRVNREY VRKVPTDIIY | |
| | DALQKAGISV EGDLRDRIAE SDGDTLKSIF YAFKYALDMR VENREEDYIQ | |
| | SPVKNASGEF FCSKNAGKSL PQDSDANGAY NIALKGILQL RMLSEQYDPN | |
| | AESIRLPLIT NKAWLTFMQS GMKTWKN | |
| WP_044910713.1 | MGLYDGFVNR YSVSKTLRFE LIPQGRTREY IETNGILSDD EERAKDYKTI | (SEQ ID |
| type V CRISPR- | KRLIDEYHKD YISRCLKNVN ISCLEEYYHL YNSSNRDKRH EELDALSDQM | NO: 162) |
| associated protein | RGEIASFLTG NDEYKEQKSR DIIINERIIN FASTDEELAA VKRFRKFTSY | |
| Cpf1 | FTGFFTNREN MYSAEKKSTA IAHRIIDVNL PKYVDNIKAF NTAIEAGVFD | |
| [Lachnospiraceae | IAEFESNFKA ITDEHEVSDL LDITKYSRFI RNEDIIIYNT LLGGISMKDE | |
| bacterium MC2017] | KIQGLNELIN LHNQKHPGKK VPLLKVLYKQ ILGDSQTHSF VDDQFEDDQQ | |
| | VINAVKAVTD TFSETLLGSL KIIINNIGHY DLDRIYIKAG QDITTLSKRA | |
| | LNDWHIITEC LESEYDDKFP KNKKSDTYEE MRNRYVKSFK SFSIGRLNSL | |
| | VTTYTEQACF LENYLGSFGG DTDKNCLTDF TNSLMEVEHL LNSEYPVTNR | |
| | LITDYESVRI LKRLLDSEME VIHFLKPLLG NGNESDKDLV FYGEFEAEYE | |
| | KLLPVIKVYN RVRNYLTRKP FSTEKIKLNF NSPTLLCGWS QSKEKEYMGV | |
| | ILRKDGQYYL GIMTPSNKKI FSEAPKPDED CYEKMVLRYI PHPYQMLPKV | |
| | FFSKSNIAFF NPSDEILRIK KQESFKKGKS FNRDDCHKFI DFYKDSINRH | |
| | EEWRKFNFKF SDTDSYEDIS RFYKEVENQA FSMSFTKIPT VYIDSLVDEG | |
| | KLYLFKLHNK DFSEHSKGKP NLHTVYWNAL FSEYNLQNTV YQLNGSAEIF | |
| | FRKASIPENE RVIHKKNVPI TRKVAELNGK KEVSVFPYDI IKNRRYTVDK | |
| | FQFHVPLKMN FKADEKKRIN DDVIEAIRSN KGIHVIGIDR GERNLLYLSL | |
| | INEEGRIIEQ RSLNIIDSGE GHTQNYRDLL DSREKDREKA RENWQEIQEI | |
| | KDLKTGYLSQ AIHTITKWMK EYNAIIVLED LNDRFTNGRK KVEKQVYQKF | |
| | EKMLIDKLNY YVDKDEEFDR MGGTHRALQL TEKFESFQKL GRQTGFIFYV | |
| | PAWNTSKLDP TTGFVDLLYP KYKSVDATKD FIKKFDFIRF NSEKNYFEFG | |
| | LHYSNFTERA IGCRDEWILC SYGNRIVNFR NAAKNNSWDY KEIDITKQLL | |
| | DLFEKNGIDV KQENLIDSIC EMKDKPFFKS LIANIKLILQ IRNSASGTDI | |
| | DYMISPAMND RGEFFDTRKG LQQLPLDADA NGAYNIAKKG LWIVDQIRNT | |
| | TGNNVKMAMS NREWMHFAQE SRLA | |
| KKQ36153.1 | MKNVFGGFTN LYSLTKTLRF ELKPTSKTQK LMKRNNVIQT DEEIDKLYHD | (SEQ ID |
| hypothetical protein | EMKPILDEIH RRFINDALAQ KIFISASLDN FLKVVKNYKV ESAKKNIKQN | NO: 163) |
| US52 C0007G0008 | QVKLLQKEIT IKTLGLRREV VSGFITVSKK WKDKYVGLGI KLKGDGYKVL | |
| [candidate division | TEQAVLDILK IEFPNKAKYI DKFRGFWTYF SGFNENRKNY YSEEDKATSI | |
| WS6 bacterium | ANRIVNENLS RYIDNIIAFE EILQKIPNLK KFKQDLDITS YNYYLNQAGI | |
| GW2011_GWA2_37_6] | DKYNKIIGGY IVDKDKKIQG INEKVNLYTQ QTKKKLPKLK FLFKQIGSER | |
| | KGFGIFEIKE GKEWEQLGDL FKLQRTKINS NGREKGLFDS LRTMYREFFD | |
| | EIKRDSNSQA RYSLDKIYFN KASVNTISNS WFTNWNKFAE LLNIKEDKKN | |
| | GEKKIPEQIS IEDIKDSLSI IPKENLEELF KLTNREKHDR TRFFGSNAWV | |
| | TFLNIWQNEI EESFNKLEEK EKDFKKNAAI KFQKNNLVQK NYIKEVCDRM | |
| | LAIERMAKYH LPKDSNLSRE EDFYWIIDNL SEQREIYKYY NAFRNYISKK | |
| | PYNKSKMKLN FENGNLLGGW SDGQERNKAG VILRNGNKYY LGVLINRGIF | |
| | RTDKINNEIY RTGSSKWERL ILSNLKFQTL AGKGFLGKHG VSYGNMNPEK | |
| | SVPSLQKFIR ENYLKKYPQL TEVSNTKFLS KKDFDAAIKE ALKECFTMNF | |
| | INIAENKLLE AEDKGDLYLF EITNKDFSGK KSGKDNIHTI YWKYLFSESN | |
| | CKSPIIGING GAEIFFREGQ KDKLHTKLDK KGKKVFDAKR YSEDKLFFHV | |
| | SITINYGKPK NIKFRDIINQ LITSMNVNII GIDRGEKHLL YYSVIDSNGI | |
| | ILKQGSLNKI RVGDKEVDFN KKLTERANEM KKARQSWEQI GNIKNFKEGY | |
| | LSQAIHEIYQ LMIKYNAIIV LEDLNTEFKA KRLSKVEKSV YKKFELKLAR | |
| | KLNHLILKDR NTNEIGGVLK AYQLTPTIGG GDVSKFEKAK QWGMMFYVRA | |
| | NYTSTTDPVT GWRKHLYISN FSNNSVIKSF FDPTNRDTGI EIFYSGKYRS | |
| | WGFRYVQKET GKKWELFATK ELERFKYNQT TKLCEKINLY DKFEELFKGI | |
| | DKSADIYSQL CNVLDFRWKS LVYLWNLLNQ IRNVDKNAEG NKNDFIQSPV | |
| | YPFFDSRKTD GKTEPINGDA NGALNIARKG LMLVERIKNN PEKYEQLIRD | |
| | TEWDAWIQNF NKVN | |
| WP_044919442.1 | MYYESLTKQY PVSKTIRNEL IPIGKTLDNI RQNNILESDV KRKQNYEHVK | (SEQ ID |
| type V CRISPR- | GILDEYHKQL INEALDNCTL PSLKIAAEIY LKNQKEVSDR EDFNKTQDLL | NO: 164) |
| associated protein | RKEVVEKLKA HENFTKIGKK DILDLLEKLP SISEDDYNAL ESFRNFYTYF | |
| Cpf1 | TSYNKVRENL YSDKEKSSTV AYRLINENFP KFLDNVKSYR FVKTAGILAD | |
| [Lachnospiraceae | GLGEEEQDSL FIVETFNKTL TQDGIDTYNS QVGKINSSIN LYNQKNQKAN | |
| bacterium MA2020] | GFRKIPKMKM LYKQILSDRE ESFIDEFQSD EVLIDNVESY GSVLIESLKS | |
| | SKVSAFFDAL RESKGKNVYV KNDLAKTAMS NIVFENWRTF DDLLNQEYDL | |
| | ANENKKKDDK YFEKRQKELK KNKSYSLEHL CNLSEDSCNL IENYIHQISD | |
| | DIENIIINNE TFLRIVINEH DRSRKLAKNR KAVKAIKDFL DSIKVLEREL | |
| | KLINSSGQEL EKDLIVYSAH EELLVELKQV DSLYNMTRNY LTKKPFSTEK | |
| | VKLNFNRSTL LNGWDRNKET DNLGVLLLKD GKYYLGIMNT SANKAFVNPP | |
| | VAKTEKVFKK VDYKLLPVPN QMLPKVFFAK SNIDFYNPSS EIYSNYKKGT | |
| | HKKGNMFSLE DCHNLIDFFK ESISKHEDWS KFGFKFSDTA SYNDISEFYR | |
| | EVEKQGYKLT YTDIDETYIN DLIERNELYL FQIYNKDFSM YSKGKLNLHT | |
| | LYFMMLFDQR NIDDVVYKLN GEAEVFYRPA SISEDELIIH KAGEEIKNKN | |
| | PNRARTKETS TFSYDIVKDK RYSKDKFTLH IPITMNFGVD EVKRFNDAVN | |
| | SAIRIDENVN VIGIDRGERN LLYVVVIDSK GNILEQISLN SIINKEYDIE | |
| | TDYHALLDER EGGRDKARKD WNTVENIRDL KAGYLSQVVN VVAKLVLKYN | |
| | AIICLEDLNF GFKRGRQKVE KQVYQKFEKM LIDKLNYLVI DKSREQTSPK | |
| | ELGGALNALQ LTSKFKSFKE LGKQSGVIYY VPAYLTSKID PTTGFANLFY | |
| | MKCENVEKSK RFFDGFDFIR FNALENVFEF GFDYRSFTQR ACGINSKWTV | |
| | CTNGERIIKY RNPDKNNMFD EKVVVVTDEM KNLFEQYKIP YEDGRNVKDM | |
| | IISNEEAEFY RRLYRLLQQT LQMRNSTSDG TRDYIISPVK NKREAYFNSE | |
| | LSDGSVPKDA DANGAYNIAR KGLWVLEQIR QKSEGEKINL AMTNAEWLEY | |
| | AQTHLL | |
| WP_035798880.1 | MYYQNLTKKY PVSKTIRNEL IPIGKTLENI RKNNILESDV KRKQDYEHVK | (SEQ ID |
| type V CRISPR- | GIMDEYHKQL INEALDNYML PSLNQAAEIY LKKHVDVEDR EEFKKTQDLL | NO: 165) |
| associated protein | RREVTGRLKE HENYTKIGKK DILDLLEKLP SISEEDYNAL ESFRNFYTYF | |
| Cpf1 [Butyrivibrio | TSYNKVRENL YSDEEKSSTV AYRLINENLP KFLDNIKSYA FVKAAGVLAD | |
| sp. NC3005] | CIEEEEQDAL FMVETFNMTL TQEGIDMYNY QIGKVNSAIN LYNQKNHKVE | |
| | EFKKIPKMKV LYKQILSDRE EVFIGEFKDD ETLLSSIGAY GNVLMTYLKS | |
| | EKINIFFDAL RESEGKNVYV KNDLSKTTMS NIVFGSWSAF DELLNQEYDL | |
| | ANENKKKDDK YFEKRQKELK KNKSYTLEQM SNLSKEDISP IENYIERISE | |
| | DIEKICIYNG EFEKIVVNEH DSSRKLSKNI KAVKVIKDYL DSIKELEHDI | |
| | KLINGSGQEL EKNLVVYVGQ EEALEQLRPV DSLYNLTRNY LTKKPFSTEK | |
| | VKLNFNKSTL LNGWDKNKET DNLGILFFKD GKYYLGIMNT TANKAFVNPP | |
| | AAKTENVFKK VDYKLLPGSN KMLPKVFFAK SNIGYYNPST ELYSNYKKGT | |
| | HKKGPSFSID DCHNLIDFFK ESIKKHEDWS KFGFEFSDTA DYRDISEFYR | |
| | EVEKQGYKLT FTDIDESYIN DLIEKNELYL FQIYNKDFSE YSKGKLNLHT | |
| | LYFMMLFDQR NLDNVVYKLN GEAEVFYRPA SIAENELVIH KAGEGIKNKN | |
| | PNRAKVKETS TFSYDIVKDK RYSKYKFTLH IPITMNFGVD EVRRENDVIN | |
| | NALRTDDNVN VIGIDRGERN LLYVVVINSE GKILEQISLN SIINKEYDIE | |
| | TNYHALLDER EDDRNKARKD WNTIENIKEL KTGYLSQVVN VVAKLVLKYN | |
| | AIICLEDLNF GFKRGRQKVE KQVYQKFEKM LIEKLNYLVI DKSREQVSPE | |
| | KMGGALNALQ LTSKFKSFAE LGKQSGIIYY VPAYLTSKID PTTGFVNLFY | |
| | IKYENIEKAK QFFDGFDFIR FNKKDDMFEF SFDYKSFTQK ACGIRSKWIV | |
| | YTNGERIIKY PNPEKNNLFD EKVINVTDEI KGLFKQYRIP YENGEDIKEI | |
| | IISKAEADFY KRLFRLLHQT LQMRNSTSDG TRDYIISPVK NDRGEFFCSE | |
| | FSEGTMPKDA DANGAYNIAR KGLWVLEQIR QKDEGEKVNL SMTNAEWLKY | |
| | AQLHLL | |
| WP_027109509.1 | MENYYDSLTR QYPVTKTIRQ ELKPVGKTLE NIKNAEIIEA DKQKKEAYVK | (SEQ ID |
| type V CRISPR- | VKELMDEFHK SIIEKSLVGI KLDGLSEFEK LYKIKTKTDE DKNRISELFY | NO: 166) |
| associated protein | YMRKQIADAL KNSRDYGYVD NKDLIEKILP ERVKDENSLN ALSCFKGFTT | |
| Cpf1 | YFTDYYKNRK NIYSDEEKHS TVGYRCINEN LLIFMSNIEV YQIYKKANIK | |
| [Lachnospiraceae | NDNYDEETLD KTFMIESFNE CLTQSGVEAY NSVVASIKTA TNLYIQKNNK | |
| bacterium NC2008] | EENFVRVPKM KVLFKQILSD RTSLFDGLII ESDDELLDKL CSFSAEVDKF | |
| | LPINIDRYIK TLMDSNNGTG IYVKNDSSLT TLSNYLTDSW SSIRNAFNEN | |
| | YDAKYTGKVN DKYEEKREKA YKSNDSFELN YIQNLLGINV IDKYIERINF | |
| | DIKEICEAYK EMTKNCFEDH DKTKKLQKNI KAVASIKSYL DSLKNIERDI | |
| | KLLNGTGLES RNEFFYGEQS TVLEEITKVD ELYNITRNYL TKKPFSTEKM | |
| | KLNFNNPQLL GGWDVNKERD CYGVILIKDN NYYLGIMDKS ANKSFLNIKE | |
| | SKNENAYKKV NCKLLPGPNK MFPKVFFAKS NIDYYDPTHE IKKLYDKGTF | |
| | KKGNSFNLED CHKLIDFYKE SIKKNDDWKN FNFNFSDTKD YEDISGFFRE | |
| | VEAQNYKITY TNVSCDFIES LVDEGKLYLF QIYNKDFSEY ATGNLNLHTL | |
| | YLKMLFDERN LKDLCIKMNG EAEVFYRPAS ILDEDKVVHK ANQKITNKNT | |
| | NSKKKESIFS YDIVKDKRYT VDKFFIHLPI TLNYKEQNVS RFNDYIREIL | |
| | KKSKNIRVIG IDRGERNLLY VVVCDSDGSI LYQRSINEIV SGSHKTDYHK | |
| | LLDNKEKERL SSRRDWKTIE NIKDLKAGYM SQVVNEIYNL ILKYNAIVVL | |
| | EDLNIGFKNG RKKVEKQVYQ NFEKALIDKL NYLCIDKTRE QLSPSSPGGV | |
| | LNAYQLTAKF ESFEKIGKQT GCIFYVPAYL TSQIDPTTGF VNLFYQKDTS | |
| | KQGLQLFFRK FKKINFDKVA SNFEFVFDYN DFTNKAEGTK TNWTISTQGT | |
| | RIAKYRSDDA NGKWISRTVH PTDIIKEALN REKINYNDGH DLIDEIVSIE | |
| | KSAVLKEIYY GFKLTLQLRN STLANEEEQE DYIISPVKNS SGNYFDSRIT | |
| | SKELPCDADA NGAYNIARKG LWALEQIRNS ENVSKVKLAI SNKEWFEYTQ | |
| | NNIPSL | |
| WP_049895985.1 | METEILKYDF FEREGKYMYY DGLTKQYALS KTIRNELVPI GKTLDNIKKN | (SEQ ID |
| type V CRISPR- | RILEADIKRK SDYEHVKKLM DMYHKKIINE ALDNFKLSVL EDAADIYFNK | NO: 167) |
| associated protein | QNDERDIDAF LKIQDKLRKE IVEQLKGHTD YSKVGNKDFL GLLKAASTEE | |
| Cpf1 [Oribacterium | DRILIESFDN FYTYFTSYNK VRSNLYSAED KSSTVAYRLI NENLPKFFDN | |
| sp. NK2B42] | IKAYRTVRNA GVISGDMSIV EQDELFEVDT FNHTLTQYGI DTYNHMIGQL | |
| WP_029202018 | NSAINLYNQK MHGAGSFKKL PKMKELYKQL LTEREEEFIE EYTDDEVLIT | |
| | SVHNYVSYLI DYLNSDKVES FFDTLRKSDG KEVFIKNDVS KTTMSNILFD | |
| | NWSTIDDLIN HEYDSAPENV KKTKDDKYFE KRQKDLKKNK SYSLSKIAAL | |
| | CRDTTILEKY IRRLVDDIEK IYTSNNVFSD IVLSKHDRSK KLSKNTNAVQ | |
| | AIKNMLDSIK DFEHDVMLIN GSGQEIKKNL NVYSEQEALA GILRQVDHIY | |
| | NLTRNYLTKK PFSTEKIKLN FNRPTFLDGW DKNKEEANLG ILLIKDNRYY | |
| | LGIMNTSSNK AFVNPPKAIS NDIYKKVDYK LLPGPNKMLP KVFFATKNIA | |
| | YYAPSEELLS KYRKGTHKKG DSFSIDDCRN LIDFFKSSIN KNTDWSTFGF | |
| | NFSDTNSYND ISDFYREVEK QGYKLSFTDI DACYIKDLVD NNELYLFQIY | |
| | NKDFSPYSKG KLNLHTLYFK MLFDQRNLDN VVYKLNGEAE VFYRPASIES | |
| | DEQIIHKSGQ NIKNKNQKRS NCKKTSTFDY DIVKDRRYCK DKFMLHLPIT | |
| | VNFGTNESGK FNELVNNAIR ADKDVNVIGI DRGERNLLYV VVVDPCGKII | |
| | EQISLNTIVD KEYDIETDYH QLLDEKEGSR DKARKDWNTI ENIKELKEGY | |
| | LSQVVNIIAK LVLKYDAIIC LEDLNFGFKR GRQKVEKQVY QKFEKMLIDK | |
| | MNYLVLDKSR KQESPQKPGG ALNALQLTSA FKSFKELGKQ TGIIYYVPAY | |
| | LTSKIDPTTG FANLFYIKYE SVDKARDFFS KFDFIRYNQM DNYFEFGFDY | |
| | KSFTERASGC KSKWIACTNG ERIVKYRNSD KNNSFDDKTV ILTDEYRSLF | |
| | DKYLQNYIDE DDLKDQILQI DSADFYKNLI KLFQLTLQMR NSSSDGKRDY | |
| | IISPVKNYRE EFFCSEFSDD TFPRDADANG AYNIARKGLW VIKQIRETKS | |
| | GTKINLAMSN SEWLEYAQCN LL | |
| WP_028248456.1 | MYYQNLTKMY PISKTLRNEL IPVGKTLENI RKNGILEADI QRKADYEHVK | (SEQ ID |
| type V CRISPR- | KLMDNYHKQL INEALQGVHL SDLSDAYDLY FNLSKEKNSV DAFSKCQDKL | NO: 168) |
| associated protein | RKEIVSLLKN HENFPKIGNK EIIKLLQSLY DNDTDYKALD SFSNFYTYFS | |
| Cpf1 | SYNEVRKNLY SDEEKSSTVA YRLINENLPK FLDNIKAYAI AKKAGVRAEG | |
| [Pseudobutyrivibrio | LSEEDQDCLF IIETFERTLT QDGIDNYNAA IGKLNTAINL FNQQNKKQEG | |
| ruminis] | FRKVPQMKCL YKQILSDREE AFIDEFSDDE DLITNIESFA ENMNVFLNSE | |
| | IITDFKIALV ESDGSLVYIK NDVSKTSFSN IVFGSWNAID EKLSDEYDLA | |
| | NSKKKKDEKY YEKRQKELKK NKSYDLETII GLFDDNSDVI GKYIEKLESD | |
| | ITAIAEAKND FDEIVLRKHD KNKSLRKNTN AVEAIKSYLD TVKDFERDIK | |
| | LINGSGQEVE KNLVVYAEQE NILAEIKNVD SLYNMSRNYL TQKPFSTEKF | |
| | KLNFNRATLL NGWDKNKETD NLGILFEKDG MYYLGIMNTK ANKIFVNIPK | |
| | ATSNDVYHKV NYKLLPGPNK MLPKVFFAQS NLDYYKPSEE LLAKYKAGTH | |
| | KKGDNFSLED CHALIDFFKA SIEKHPDWSS FGFEFSETCT YEDLSGFYRE | |
| | VEKQGYKITY TDVDADYITS LVERDELYLF QIYNKDFSPY SKGNLNLHTI | |
| | YLQMLFDQRN LNNVVYKLNG EAEVFYRPAS INDEEVIIHK AGEEIKNKNS | |
| | KRAVDKPTSK FGYDIIKDRR YSKDKFMLHI PVTMNFGVDE TRRFNDVVND | |
| | ALRNDEKVRV IGIDRGERNL LYVVVVDTDG TILEQISLNS IINNEYSIET | |
| | DYHKLLDEKE GDRDRARKNW TTIENIKELK EGYLSQVVNV IAKLVLKYNA | |
| | IICLEDLNFG FKRGRQKVEK QVYQKFEKML IDKLNYLVID KSRKQDKPEE | |
| | FGGALNALQL TSKFTSFKDM GKQTGIIYYV PAYLTSKIDP TTGFANLFYV | |
| | KYENVEKAKE FFSRFDSISY NNESGYFEFA FDYKKFTDRA CGARSQWTVC | |
| | TYGERIIKFR NTEKNNSFDD KTIVLSEEFK ELFSIYGISY EDGAELKNKI | |
| | MSVDEADFFR SLTRLFQQTM QMRNSSNDVT RDYIISPIMN DRGEFFNSEA | |
| | CDASKPKDAD ANGAFNIARK GLWVLEQIRN TPSGDKLNLA MSNAEWLEYA | |
| | QRNQI | |
| WP_028830240 | MENFKNLYPI NKTLRFELRP YGKTLENFKK SGLLEKDAFK ANSRRSMQAI | (SEQ ID |
| type V CRISPR- | IDEKFKETIE ERLKYTEFSE CDLGNMTSKD KKITDKAATN LKKQVILSFD | NO: 169) |
| associated protein | DEIFNNYLKP DKNIDALFKN DPSNPVISTF KGFTTYFVNF FEIRKHIFKG | |
| Cpf1 [Proteocatella | ESSGSMAYRI IDENLTTYLN NIEKIKKLPE ELKSQLEGID QIDKLNNYNE | |
| sphenisci] | FITQSGITHY NEIIGGISKS ENVKIQGINE GINLYCQKNK VKLPRLTPLY | |
| | KMILSDRVSN SFVLDTIEND TELIEMISDL INKTEISQDV IMSDIQNIFI | |
| | KYKQLGNLPG ISYSSIVNAI CSDYDNNFGD GKRKKSYEND RKKHLETNVY | |
| | SINYISELLT DTDVSSNIKM RYKELEQNYQ VCKENFNATN WMNIKNIKQS | |
| | EKTNLIKDLL DILKSIQRFY DLFDIVDEDK NPSAEFYTWL SKNAEKLDFE | |
| | FNSVYNKSRN YLTRKQYSDK KIKLNFDSPT LAKGWDANKE IDNSTIIMRK | |
| | FNNDRGDYDY FLGIWNKSTP ANEKIIPLED NGLFEKMQYK LYPDPSKMLP | |
| | KQFLSKIWKA KHPTTPEFDK KYKEGRHKKG PDFEKEFLHE LIDCFKHGLV | |
| | NHDEKYQDVF GFNLRNTEDY NSYTEFLEDV ERCNYNLSFN KIADTSNLIN | |
| | DGKLYVFQIW SKDFSIDSKG TKNLNTIYFE SLFSEENMIE KMFKLSGEAE | |
| | IFYRPASLNY CEDIIKKGHH HAELKDKFDY PIIKDKRYSQ DKFFFHVPMV | |
| | INYKSEKLNS KSLNNRTNEN LGQFTHIIGI DRGERHLIYL TVVDVSTGEI | |
| | VEQKHLDEII NTDTKGVEHK THYLNKLEEK SKTRDNERKS WEAIETIKEL | |
| | KEGYISHVIN EIQKLQEKYN ALIVMENLNY GFKNSRIKVE KQVYQKFETA | |
| | LIKKFNYIID KKDPETYIHG YQLTNPITTL DKIGNQSGIV LYIPAWNTSK | |
| | IDPVTGFVNL LYADDLKYKN QEQAKSFIQK IDNIYFENGE FKFDIDFSKW | |
| | NNRYSISKTK WTLTSYGTRI QTFRNPQKNN KWDSAEYDLT EEFKLILNID | |
| | GTLKSQDVET YKKFMSLFKL MLQLRNSVTG TDIDYMISPV TDKTGTHFDS | |
| | RENIKNLPAD ADANGAYNIA RKGIMAIENI MNGISDPLKI SNEDYLKYIQ | |
| | NQQE | |
| WP_084502895.1 | MIILYISTSN MNMEGVFMEN FKNLYPINKT LRFELRPYGK TLENFKKSGL | (SEQ ID |
| type V CRISPR- | LEKDAFKANS RRSMQAIIDE KFKETIEERL KYTEFSECDL GNMTSKDKKI | NO: 170) |
| associated protein | TDKAATNLKK QVILSFDDEI FNNYLKPDKN IDALFKNDPS NPVISTFKGF | |
| Cpf1 [Proteocatella | TTYFVNFFEI RKHIFKGESS GSMAYRIIDE NLTTYLNNIE KIKKLPEELK | |
| sphenisci] | SQLEGIDQID KLNNYNEFIT QSGITHYNEI IGGISKSENV KIQGINEGIN | |
| | LYCQKNKVKL PRLTPLYKMI LSDRVSNSFV LDTIENDTEL IEMISDLINK | |
| | TEISQDVIMS DIQNIFIKYK QLGNLPGISY SSIVNAICSD YDNNFGDGKR | |
| | KKSYENDRKK HLETNVYSIN YISELLTDTD VSSNIKMRYK ELEQNYQVCK | |
| | ENFNATNWMN IKNIKQSEKT NLIKDLLDIL KSIQRFYDLF DIVDEDKNPS | |
| | AEFYTWLSKN AEKLDFEFNS VYNKSRNYLT RKQYSDKKIK LNFDSPTLAK | |
| | GWDANKEIDN STIIMRKFNN DRGDYDYFLG IWNKSTPANE KIIPLEDNGL | |
| | FEKMQYKLYP DPSKMLPKQF LSKIWKAKHP TTPEFDKKYK EGRHKKGPDF | |
| | EKEFLHELID CFKHGLVNHD EKYQDVFGFN LRNTEDYNSY TEFLEDVERC | |
| | NYNLSFNKIA DTSNLINDGK LYVFQIWSKD FSIDSKGTKN LNTIYFESLF | |
| | SEENMIEKMF KLSGEAEIFY RPASLNYCED IIKKGHHHAE LKDKFDYPII | |
| | KDKRYSQDKF FFHVPMVINY KSEKLNSKSL NNRTNENLGQ FTHIIGIDRG | |
| | ERHLIYLTVV DVSTGEIVEQ KHLDEIINTD TKGVEHKTHY LNKLEEKSKT | |
| | RDNERKSWEA IETIKELKEG YISHVINEIQ KLQEKYNALI VMENLNYGFK | |
| | NSRIKVEKQV YQKFETALIK KFNYIIDKKD PETYIHGYQL TNPITTLDKI | |
| | GNQSGIVLYI PAWNTSKIDP VTGFVNLLYA DDLKYKNQEQ AKSFIQKIDN | |
| | IYFENGEFKF DIDFSKWNNR YSISKTKWTL TSYGTRIQTF RNPQKNNKWD | |
| | SAEYDLTEEF KLILNIDGTL KSQDVETYKK FMSLFKLMLQ LRNSVTGTDI | |
| | DYMISPVTDK TGTHFDSREN IKNLPADADA NGAYNIARKG IMAIENIMNG | |
| | ISDPLKISNE DYLKYIQNQQ E | |
| WP 055225123.1 | MNNGTNNFQN FIGISSLQKT LRNALIPTET TQQFIVKNGI IKEDELRGEN | (SEQ ID |
| Eubacterium rectale | RQILKDIMDD YYRGFISETL SSIDDIDWTS LFEKMEIQLK NGDNKDTLIK | NO: 171) |
| | EQTEYRKAIH KKFANDDRFK NMFSAKLISD ILPEFVIHNN NYSASEKEEK | |
| | TQVIKLFSRF ATSFKDYFKN RANCFSADDI SSSSCHRIVN DNAEIFFSNA | |
| | LVYRRIVKSL SNDDINKISG DMKDSLKEMS LEEIYSYEKY GEFITQEGIS | |
| | FYNDICGKVN SFMNLYCQKN KENKNLYKLQ KLHKQILCIA DTSYEVPYKF | |
| | ESDEEVYQSV NGFLDNISSK HIVERLRKIG DNYNGYNLDK IYIVSKFYES | |
| | VSQKTYRDWE TINTALEIHY NNILPGNGKS KADKVKKAVK NDLQKSITEI | |
| | NELVSNYKLC SDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HIKSSKDFDI TFCHDLIDYF KNCIAIHPEW | |
| | KNFGFDFSDT STYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKNIPE NIYQELYKYF | |
| | NDKSDKELSD EAAKLKNVVG HHEAATNIVK DYRYTYDKYF LHMPITINFK | |
| | ANKTGFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPDKL KNVGHQCGCI FYVPAAYTSK IDPTTGFVNI | |
| | FKFKDLTVDA KREFIKKFDS IRYDSEKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFRLTV QMRNSLSELE DRDYDRLISP VLNENNIFYD | |
| | SAKAGDALPK DADANGAYCI ALKGLYEIKQ ITENWKEDGK FSRDKLKISN | |
| | KDWFDFIQNK RYL | |
| WP_055237260.1 | MNNGTNNFQN FIGISSLQKT LRNALIPTET TQQFIVKNGI IKEDELRGEN | (SEQ ID |
| Eubacterium rectale | RQILKDIMDD YYRGFISETL SSIDDIDWTS LFEKMEIQLK NGDNKDTLIK | NO: 172) |
| | EQAEKRKAIY KKFADDDRFK NMFSAKLISD ILPEFVIHNN NYSASEKEEK | |
| | TQVIKLFSRI ATSFKDYFKN RANCFSADDI SSSSCHRIVN DNAEIFFSNA | |
| | LVYRRIVKNL SNDDINKISG DMKDSLKEMS LDEIYSYEKY GEFITQEGIS | |
| | FYNDICGKVN SFMNLYCQKN KENKNLYKLR KLHKQILCIA DTSYEVPYKF | |
| | ESDEEVYQSV NGFLDNISSK HIVERLRKIG DNYNGYNLDK IYIVSRFYES | |
| | VSQKTYRDWE TINTALEIHY NNILPGNGKS KADKVKKAVK NDLQKSITEI | |
| | NELVSNYKLC PDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HLKSSKDFDI TFCRDLIDYF KNCIAIHPEW | |
| | KNFGFDFSDT STYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKTIPE NIYQELYKYF | |
| | NDKSDKELSD EAAKLKNVVG HHEAATNIVK DYRYTYDKYF LHMPITINFK | |
| | ANKTSFINDR ILQYIAKEND LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPEKL KNVGHQCGCI FYVPAAYTSK IDPTTGFANI | |
| | FKFKDLTVDA KREFIKKFDS IRYDSEKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFKLTV QMRNSLSELE DRDYDRLISP VLNENNIFYD | |
| | SAKAGDALPK DADANGAYCI ALKGLYEIKQ ITENWKEDGK FSRDKLKISN | |
| | KDWFDFIQNK RYL | |
| WP_055272206.1 | MNNGTNNFQN FIGISSLQKT LRNALTPTET TQQFIVKNGI IKEDELRGEN | (SEQ ID |
| Eubacterium rectale | RQILKDIMDD YYRGFISETL SSIDDIDWTS LFEKMEIQLK NGDNKDTLIK | NO: 173) |
| | EQAEKRKAIY KKFADDDRFK NMFSAKLISD ILPEFVIHNN NYSASEKEEK | |
| | TQVIKLFSRF ATSFKDYFKN RANCFSADDI SSSSCHRIVN DNAEIFFSNA | |
| | LVYRRIVKNL SNDDINKISG DMKDSLKKMS LEKIYSYEKY GEFITQEGIS | |
| | FYNDICGKVN SFMNLYCQKN KENKNLYKLR KLHKQILCIA DTSYEVPYKF | |
| | ESDEEVYQSV NGFLDNISSK HIVERLRKIG DNYNGYNLDK IYIVSKFYES | |
| | VSQKTYRDWE TINTALEIHY NNILPGNGKS KADKVKKAVK NDLQKSITEI | |
| | NELVSNYKLC PDDNIKAETY IHEISHILNN FEAQELKYNP EIHLVESELK | |
| | ASELKNVLDV IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PEKKIIEGNT SENKGDYKKM IYNLLPGPNK MIPKVFLSSK | |
| | TGVETYKPSA YILEGYKQNK HLKSSKDFDI TFCRDLIDYF KNCIAIHPEW | |
| | KNFGFDFSDT STYEDISGFY REVELQGYKI DWTYISEKDI DLLQEKGQLY | |
| | LFQIYNKDFS KKSTGNDNLH TMYLKNLFSE ENLKDVVLKL NGEAEIFFRK | |
| | SSIKNPIIHK KGSILVNRTY EAEEKDQFGN IQIVRKTIPE NIYQELYKYF | |
| | NDKSDKELSD EAAKLKNAVG HHEAATNIVK DYRYTYDKYF LHMPITINFK | |
| | ANKTSFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPEKL KNVGHQCGCI FYVPAAYTSK IDPTTGFVNI | |
| | FKFKDLTVDA KREFIKKFDS IRYDSDKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFKLTV QMRNSLSELE DRNYDRLISP VLNENNIFYD | |
| | SAKAGDALPK DADANGAYCI ALKGLYEIKQ ITENWKEDGK FSRDKLKISN | |
| | KDWFDFIQNK RYL | |
| OLA16049.1 | MNNGTNNFQN FIGISSLQKT LRNALIPTET TQQFIVKNGI IKEDELRGKN | (SEQ ID |
| Eubacterium sp. | RQILKDIMDD YYRGFISETL SSIDDIDWTS LFEKMEIQLK NGDNKDTLIK | NO: 174) |
| 41_20 | EQAEKRKAIY KKFADDDRFK NMFSAKLISD ILPEFVIHNN NYSASEKKEK | |
| | TQVIKLFSRF ATSFKDYFKN RANCFSADDI SSSSCHRIVN DNAEIFFSNA | |
| | LVYRRIVKNL SNDDINKISG DMKDSLKEMS LEEIYSYEKY GEFITQEGIS | |
| | FYNDICGKVN SFMNLYCQKN KENKNLYKLR KLHKQILCIA DTSYEVPYKF | |
| | ESDEEVYQSV NGFLDNISSK HIVERLRKIG DNYNDYNLDK IYIVSKFYES | |
| | VSQKTYRDWE TINTALEIHY NNILPGNGKS KADKVKKAVK NDLQKSITEI | |
| | NELVSNYKLC SDDNIKAETY IHEISHILNN FEAHELKYNP EIHLVESELK | |
| | ASELKNVLDI IMNAFHWCSV FMTEELVDKD NNFYAELEEI YDEIYPVISL | |
| | YNLVRNYVTQ KPYSTKKIKL NFGIPTLADG WSKSKEYSNN AIILMRDNLY | |
| | YLGIFNAKNK PDKKIIEGNT SENKGDYKKM IYNLLPGPNK MIPKVFLSSK | |
| | TGVETYKPSA YILEGYKQNK HLKSSKDFDI TFCHDLIDYF KNCIAIHPEW | |
| | KNFGFDFSDT SAYEDISGFY REVELQGYKI DWTYISEKDI DLLQEKGQLY | |
| | LFQIYNKDFS KKSTGNDNLH TMYLKNLFSE ENLKDIVLKL NGEAEIFFRK | |
| | SSIKNPIIHK KGSILVNRTY EAEEKDQFGN IQIVRKTIPE NIYQELYKYF | |
| | NDKSDKELSD EAAKLKNVVG HHEAATNIVK DYRYTYDKYF LHMPITINFK | |
| | ANKTSFINDR ILQYIAKEKD LHVIGIDRGE RNLIYVSVID TCGNIVEQKS | |
| | FNIVNGYDYQ IKLKQQEGAR QIARKEWKEI GKIKEIKEGY LSLVIHEISK | |
| | MVIKYNAIIA MEDLSYGFKK GRFKVERQVY QKFETMLINK LNYLVFKDIS | |
| | ITENGGLLKG YQLTYIPDKL KNVGHQCGCI FYVPAAYTSK IDPTTGFVNI | |
| | FKFKDLTVDA KREFIKKFDS IRYDSEKNLF CFTFDYNNFI TQNTVMSKSS | |
| | WSVYTYGVRI KRRFVNGRFS NESDTIDITK DMEKTLEMTD INWRDGHDLR | |
| | QDIIDYEIVQ HIFEIFKLTV QMRNSLSELE DRDYDRLISP VLNENNIFYD | |
| | SAKAGYALPK DADANGAYCI ALKGLYEIKQ ITENWKEDGK FSRDKLKISN | |
| | KDWFDFIQNK RYL | |

TABLE 6
Cas12b (C2c1) orthologs

| | | |
|---|---|---|
| Alicyclobacillus | MVAVKSIKVK LMLGHLPEIR EGLWHLHEAV NLGVRYYTEW LALLRQGNLY | (SEQ ID |
| macrosporangiidus | RRGKDGAQEC YMTAEQCRQE LLVRLRDRQK RNGHTGDPGT DEELLGVARR | NO: 175) |
| strain DSM 17980 | LYELLVPQSV GKKGQAQMLA SGFLSPLADP KSEGGKGTSK SGRKPAWMGM | |
| WP_074948407.1 | KEAGDSRWVE AKARYEANKA KDPTKQVIAS LEMYGLRPLF DVFTETYKTI | |
| | RWMPLGKHQG VRAWDRDMFQ QSLERLMSWE SWNERVGAEF ARLVDRRDRF | |
| | REKHFTGQEH LVALAQRLEQ EMKEASPGFE SKSSQAHRIT KRALRGADGI | |
| | IDDWLKLSEG EPVDRFDEIL RKRQAQNPRR FGSHDLFLKL AEPVFQPLWR | |
| | EDPSFLSRWA SYNEVLNKLE DAKQFATFTL PSPCSNPVWA RFENAEGTNI | |
| | FKYDFLFDHF GKGRHGVRFQ RMIVMRDGVP TEVEGIVVPI APSRQLDALA | |
| | PNDAASPIDV FVGDPAAPGA FRGQFGGAKI QYRRSALVRK GRREEKAYLC | |
| | GFRLPSQRRT GTPADDAGEV FLNLSLRVES QSEQAGRRNP PYAAVFHISD | |
| | QTRRVIVRYG EIERYLAEHP DTGIPGSRGL TSGLRVMSVD LGLRTSAAIS | |
| | VFRVAHRDEL TPDAHGRQPF FFPIHGMDHL VALHERSHLI RLPGETESKK | |
| | VRSIREQRLD RLNRLRSQMA SLRLLVRTGV LDEQKRDRNW ERLQSSMERG | |
| | GERMPSDWWD LFQAQVRYLA QHRDASGEAW GRMVQAAVRT LWRQLAKQVR | |
| | DWRKEVRRNA DKVKIRGIAR DVPGGHSLAQ LDYLERQYRF LRSWSAFSVQ | |
| | AGQVVRAERD SRFAVALREH IDNGKKDRLK KLADRILMEA LGYVYVTDGR | |
| | RAGQWQAVYP PCQLVLLEEL SEYRFSNDRP PSENSQLMVW SHRGVLEELI | |
| | HQAQVHDVLV GTIPAAFSSR FDARTGAPGI RCRRVPSIPL KDAPSIPIWL | |
| | SHYLKQTERD AAALRPGELI PTGDGEFLVT PAGRGASGVR VVHADINAAH | |
| | NLQRRLWENF DLSDIRVRCD RREGKDGTVV LIPRLTNQRV KERYSGVIFT | |
| | SEDGVSFTVG DAKTRRRSSA SQGEGDDLSD EEQELLAEAD DARERSVVLF | |
| | RDPSGFVNGG RWTAQRAFWG MVHNRIETLL AERFSVSGAA EKVRG | |
| Bacillus hisashii | MATRSFILKI EPNEEVKKGL WKTHEVLNHG IAYYMNILKL IRQEAIYEHH | (SEQ ID |
| strain C4 | EQDPKNPKKV SKAEIQAELW DFVLKMQKCN SFTHEVDKDE VFNILRELYE | NO: 176) |
| WP_095142515.1 | ELVPSSVEKK GEANQLSNKF LYPLVDPNSQ SGKGTASSGR KPRWYNLKIA | |
| | GDPSWEEEKK KWEEDKKKDP LAKILGKLAE YGLIPLFIPY TDSNEPIVKE | |
| | IKWMEKSRNQ SVRRLDKDMF IQALERFLSW ESWNLKVKEE YEKVEKEYKT | |
| | LEERIKEDIQ ALKALEQYEK ERQEQLLRDT LNTNEYRLSK RGLRGWREII | |
| | QKWLKMDENE PSEKYLEVFK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDFPKVVNF KPKELTEWIK | |
| | DSKGKKLKSG IESLEIGLRV MSIDLGQRQA AAASIFEVVD QKPDIEGKLF | |
| | FPIKGTELYA VHRASFNIKL PGETLVKSRE VLRKAREDNL KLMNQKLNFL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYEERS RFENSKLMKW | |
| | SRREIPRQVA LQGEIYGLQV GEVGAQFSSR FHAKTGSPGI RCSVVTKEKL | |
| | QDNRFFKNLQ REGRLTLDKI AVLKEGDLYP DKGGEKFISL SKDRKCVTTH | |
| | ADINAAQNLQ KRFWTRTHGF YKVYCKAYQV DGQTVYIPES KDQKQKIIEE | |
| | FGEGYFILKD GVYEWVNAGK LKIKKGSSKQ SSSELVDSDI LKDSFDLASE | |
| | LKGEKLMLYR DPSGNVFPSD KWMAAGVFFG KLERILISKL TNQYSISTIE | |
| | DDSSKQSM | |
| Candidatus | MPRDDLDLLT NLNSTAKGIR ERGKTKEGTD KKKSGRKSSW PMDKAAWETA | (SEQ ID |
| Lindowbacteria | KTSDSSAHFL EKLKQHPDLK DAFGNLSSGG SKKLEYYKKL AGSAPWKESQ | NO: 177) |
| bacterium | SVILEKAARW KEAKQEREEK EQDSSEHGSK AAYRRLFDAG CLPMPEFAKY | |
| RIFCSPLOWO2 | IDENQIEFGD LKLSDCGAEW KRGMWNQAGQ RVRSHMGWQR RREKENAVYS | |
| OGH55994.1 | LRKELFEKGG AIRRKKSEEL TPEDILPGKA APDQNDWQER PAYGNQMWFI | |
| | GLRSYEENEM AKYAEEAGMG SRSAPRIRRG TIKGWSKLRE RWLQILKRNP | |
| | QATRDDLIGE LNALRSQDPR AYGDARLFDW LSKTDQRFLW DGFDADGKIL | |
| | CGRDDRDCVS AFVAYNEEFA DEPSSITLTE TDERLHPVWP FFGESSAVPY | |
| | EIEYDLETAC PTAIRLPLLV GKENGGYAER QGTRIPLAEY ADLASSFQLP | |
| | TPVRLDVLVE IREVTRAGRK VTCPFSYFKQ NGVWYVREGE IPSGESIQIK | |
| | QTDRKIENGK IFISSKLRMA YRDDLMVSPA TGDFGSIKIL WERIELASHV | |
| | DQKKLPETAP ARSRVFVSFS CNVVERAPRK QLTRKPDAVV VTIPSGVDQG | |
| | LVVVSTDVRT GKSKSSSAPP LPPGSRLWPA DAVHGDPPLR ILSVDLGHRH | |
| | SAYAVWELGL QQKSWRAGVL KGSTQTPVYA DCTGTGLLCL PGDGEDTPAE | |
| | EESLRLRSRQ IRRRLNLQNS ILRVSRLLSL DKFEKTIFEQ SDVRDRPNKK | |
| | GLRIRRRCRT EKTPLSEAEV RKNCDKAAEI LIRWADTDAM AKSLAATGNA | |
| | DISFWKYMAV KNPPLSAVVD VAPSTIVPDD GPDRETLKKK RQEEEEKFAS | |
| | SIYENRVKLA GALCSGYDAD HRRPATGGLW HDLDRTLIRE ISYGDRGQKG | |
| | NPRKLNNEGI LRLLRRPPRA RPDWREFHRT LNDANRIPKG RTLRGGLSMG | |
| | RLNFLKEVGD FVKKWSCRPR WPGDRRHIPP GQLFDRQDAE HLEHLRDDRI | |
| | KRLAHLIVAQ ALGFEPDIRR GLWKYVDGST GEILWQHPET RRFFAEGAAG | |
| | ELREVSRPAE IDDDAAARPH TVSAPAHIVV FENLIRYRFQ SDRPKTENAG | |
| | LMQWAHRQIV HFTKQVASLY GLKVAMVYAA FSSKFCSRCG SPGARVSRFD | |
| | PAWRNQEWFK RRTSNPRSKV DHSLKRASED PTADETRPWV LIEGGKEFVC | |
| | ANAKCSAHDE PLNADENAAA NIGLRFLRGV EDFRTKVNPA GALKGKLRFE | |
| | TGIHSFRPPV SGSPFWSPMA EPAQKKKIGA AAPGADVDEA GDADESGVVV | |
| | LFRDPSGAFR NKQYWYEGKI FWSNVMMAVE AKIAGASVGA KPVAASWGQA | |
| | QPQSGPGLAK PGGD | |
| Elusimicrobia | MNRIYQGRVT KVEVPDGKDE KGNIKWKKLE NWSDILWQHH MLFQDAVNYY | (SEQ ID |
| bacterium | TLALAAISGS AVGSDEKSII LREWAVQVQN IWEKAKKKAT VFEGPQKRLT | NO: 178) |
| RIFOXYA12 | SILGLEQNAS FDIAAKHILR TSEAKPEQRA SALIRLLEEI DKKNHNVVCG | |
| OGS02326.1 | ERLPFFCPRN IQSKRSPTSK AVSSVQEQKR QEEVRRFHNM QPEEVVKNAV | |
| | TLDISLFKSS PKIVFLEDPK KARAELLKQF DNACKKHKEL VGIKKAFTES | |
| | IDKHGSSLKV PAPGSKPSGL YPSAIVFKYF PVDITKTVFL KATEKLAMGK | |
| | DREVTNDPIA DARVNDKPHF DYFTNIALIR EKEKNRAAWF EFDLAAFIEA | |
| | IMSPHRFYQD TQKRKEAARK LEEKIKAIEG KGGQFKESDS EDDDVDSLPG | |
| | FEGDTRIDLL RKLVTDTLGW LGESETPDNN EGKKTEYSIS ERTLRIFPDI | |
| | QKQWSELAEK GETTEGKLLE VLKHEQTEHQ SDFGSATLYQ HLAKPEFHPI | |
| | WLKSGTEEWH AENPLKAWLN YKELQYELTD KKRPIHFTPA HPVYSPRYFD | |
| | FPKKSETEEK EVSKNTHSLT TSLASEHIKN SLQFTAGLIR KTNVGKKAIK | |
| | ARFSYSAPRL RRDCLRSENN ENLYKAPWLQ PMMRALGIDE EKADRQNFAN | |
| | TRITLMAKGI DDIQLGFPVE ANSQELQKEV SNGISWKGQF NWGGIASLSA | |
| | LRWPHEKKPK NPPEQPWWGI DSFSCLAVDL GQRYAGAFAR LDVSTIEKKG | |
| | KSRFIGEACD KKWYAKVSRM GLLRLPGEDV KVWRDASKID KENGFAFRKE | |
| | LFGEKGRSAT PLEAEETAEL IKLFGANEKD VMPDNWSKEL SFPEQNDKLL | |
| | IVARRAQAAV SRLHRWAWFF DEAKRSDDAI REILESDDTD LKQKVNKNEI | |
| | EKVKETIISL LKVKQELLPT LLTRLANRVL PLRGRSWEWK KHHQKNDGFI | |
| | LDQTGKAMPN VLIRGQRGLS MDRIEQITEL RKRFQALNQS LRRQIGKKAP | |
| | AKRDDSIPDC CPDLLEKLDH MKEQRVNQTA HMILAEALGL KLAEPPKDKK | |
| | ELNETCDMHG AYAKVDNPVS FIVIEDLSRY RSSQGRSPRE NSRLMKWCHR | |
| | AVRDKLKEMC EVFFPLCERR KAGSAWVSLP PLLETPAAYS SRFCSRSGVA | |
| | GFRAVEVIPG FELKYPWSWL KDKKDKAGNL AKEALNIRTV SEQLKAFNQD | |
| | KPEKPRTLLV PIAGGPIFVP ISEVGLSSFG LKPQVVQADI NAAINLGLRA | |
| | ISDPRIWEIH PRLRTEKRDG RLFAREKRKY GEEKVEVQPS KNEKAKKVKD | |
| | DRKPNYFADF SGKVDWGFGN IKNESGLTLV SGKALWWTIN QLQWERCFDI | |
| | NKRHIEDWSN KQKQ | |
| Omnitrophica | MNRIYQGRVT KVEKLKNGKS PDDREELKDW QTALWRHHEL FQDAVSYYTL | (SEQ ID |
| WOR_2 bacterium | ALAAMAEGLP DKHPINVLRK RMEEAWEEFP RKTVTPAKNL RDSVRPWLGL | NO: 179) |
| RIFCSPHIGHO2 | SESASFGDAL KKILPPAPEN KEVRALAVAL LAEKARTLKP QKTSASYWGR | |
| OGX36711.1 | FCDDLKKKPN WDYSEEELAR KTGSGDWVAG LWSEDALNKI DELAKSLKLS | |
| | SLVKCVPDGQ INPEGARNLV KEALDHLEGV SNGTKKEKND PGPAKKTNNW | |
| | LRQHASDVRN FIHKNKNQFS SLPNGRLITE RARGGGININ KTYAGVLFKA | |
| | FPCPFTFDYV RAAVPEPKVK KVDQEKKSEQ SATWTELEKR ILRIGDDPIE | |
| | LARKNNKPIF KAFTALEKWS DQNSKSCWSD FDKCAFEEAL KTLNQFNQKT | |
| | EEREKRRSEA EAELKYMMDE NPEWKPKKET EGDDVREVPI LKGDPRYEKL | |
| | VKLFGDLDEE GSEHATGKIY GPSRASLRGF GKLRNEWVDL FTKANDNPRE | |
| | QDLQKAVTGF QREHKLDMGY TAFFLKLCER DYWDIWRDDT EVEVKKIREK | |
| | RWVKSVVYAA ADTRELAEEL ERLQEPVRYT PAEPQFSRRL FMFSDIKGKQ | |
| | GAKHIREGLV EVSLAVKDQS GKYGTCRVRL HYSAPRLIRD HLSDGSSSMW | |
| | LQPMMAALGL SSDARGCFTR DSKGNVKEPA VALMSDFVGR KRELRMLLNF | |
| | PVDLDISKLE ENIGKKARWE KQMNTAYEKN KLKQRFHLIW PGMELKETQE | |
| | PGQFWWDNPT IQKEGMYCLA IDLSQRRAAD YALLHAGVNR DSKTFVELGQ | |
| | AGGQSWFTKL CAAGSLRLPG EDTEVIREGK RQIELSGKKG RNATQSEYDQ | |
| | AIALAKQLLH NENSAELESA ARDWLGDNAK RFSFPEQNDK LIDLYYGALS | |
| | RYKTWLRWSW RLTEQHKELW DKTLDEIRKV PYFASWGELA GNGTNEATVQ | |
| | QLQKLIADAA VDLRNFLEKA LLHIAYRALP LRENTWRWIE NGKDGKGKPL | |
| | HLLVSDGQSP AEIPWLRGQR GLSIARIEQL ENFRRAVLSL NRLLRHEIGT | |
| | KPEFGSSTCG ESLPDPCPDL TDKIVRLKEE RVNQTAHLII AQSLGVRLKG | |
| | HSLFTEEREK ADMHGEHEVI PGRSPVDFVV LEDLSRYTTD KSRSRSENSR | |
| | LMKWCHRKIN EKVKLLAEPF GIPVIEVFAS YSSKFDARTG APGFRAVEVT | |
| | SEDRPFWRKT IEKQSVAREV FDCLDNLVGK GLNGIHLVLP QNGGPLFIAA | |
| | VKEDQPLPAI RQADINAAVN IGLRAIAGPS CYHAHPKVRL IKGESGTDKG | |
| | KWLPRKGKEA NKRENAQFGN VDLDLEVKFN RLDIDSDVLK GDNTNLFHDP | |
| | LNIACYGFAT IQNLQHPFLA HASAVFSRQK GAVARLQWEV CRAINSRRLE | |
| | AWQKKAEKAA VKR | |
| Phycisphaerae | MATKSYRARI LTDSRLAAAL DRTHVVFVES LKQMINTYLR MQNGKFGPDH | (SEQ ID |
| bacterium ST- | KKLAQIMLSR SNTFAHGVMD QITRDQPTST LDEEWTDLAR RIHKTTGPLF | NO: 180) |
| NAGAB-D1 | LQAERFATVK NRAIHTKSRG KVIPSPETLA VPAKFWHQVC DSASAYIRSN | |
| (transposase) | RELMQQWRKD RAAWLKDKNE WQQKHPEFMQ FYNGPYQNFL KLCDDDRITS | |
| AQT69685.1 | QLAAEQQPTA SKNNRPRKTG KRFARWHLWY KWLSENPEII EWRNKASASD | |
| | FKTVTDDVRK QIITKYPQQN KYITRLLDWL EDNNPELKTL ENLRRTYVKK | |
| | FDSFKRPPTL TLPSPYRHPY WFTMELDQFY KKADFENGTI QLLLIDEDDD | |
| | GNWFFNWMPA SLKPDPRLVP SWRAETFETE GRFPPYLGGK IGKKLSRPAP | |
| | TDAERKAGIA GAKLMIKNNR SELLFTVFEQ DCPPRVKWAK TKNRKCPADN | |
| | AFSSDGKTRK PLRILSIDLG IRHIGAFALT QGTRNDSAWQ TESLKKGIIN | |
| | SPSIPPLRQV RRHDYDLKRK RRRHGKPVKG QRSNANLQAH RTNMAQDRFK | |
| | KGASAIVSLA REHSADLILF ENLHSLKFSA FDERWMNRQL RDMNRRHIVE | |
| | LVSEQAPEFG ITVKDDINPW MTSRICSNCN LPGFRFSMKK KNPYREKLPR | |
| | EKCTDFGYPV WEPGGHLFRC PHCDHRVNAD INAAANLANK FFGLGYWNNG | |
| | LKYDAETKTF TVHTDKKTPP LIFKPRPQFD LWADSVKTRK QLGPDPF | |
| Planctomycetes | MSVRSFQARV ECDKQTMEHL WRTHKVFNER LPEIIKILFK MKRGECGQND | (SEQ ID |
| bacterium | KQKSLYKSIS QSILEANAQN ADYLLNSVSI KGWKPGTAKK YRNASFTWAD | NO: 181) |
| RBG_13_46_10 | DAAKLSSQGI HVYDKKQVLG DLPGMMSQMV CRQSVEAISG HIELTKKWEK | |
| OHB62175.1 | EHNEWLKEKE KWESEDEHKK YLDLREKFEQ FEQSIGGKIT KRRGRWHLYL | |
| | KWLSDNPDFA AWRGNKAVIN PLSEKAQIRI NKAKPNKKNS VERDEFFKAN | |
| | PEMKALDNLH GYYERNFVRR RKTKKNPDGF DHKPTFTLPH PTIHPRWFVF | |
| | NKPKTNPEGY RKLILPKKAG DLGSLEMRLL TGEKNKGNYP DDWISVKFKA | |
| | DPRLSLIRPV KGRRVVRKGK EQGQTKETDS YEFFDKHLKK WRPAKLSGVK | |
| | LIFPDKTPKA AYLYFTCDIP DEPLTETAKK IQWLETGDVT KKGKKRKKKV | |
| | LPHGLVSCAV DLSMRRGTTG FATLCRYENG KIHILRSRNL WVGYKEGKGC | |
| | HPYRWTEGPD LGHIAKHKRE IRILRSKRGK PVKGEESHID LQKHIDYMGE | |
| | DRFKKAARTI VNFALNTENA ASKNGFYPRA DVLLLENLEG LIPDAEKERG | |
| | INRALAGWNR RHLVERVIEM AKDAGFKRRV FEIPPYGTSQ VCSKCGALGR | |
| | RYSIIRENNR REIRFGYVEK LFACPNCGYC ANADHNASVN LNRRFLIEDS | |
| | FKSYYDWKRL SEKKQKEEIE TIESKLMDKL CAMHKISRGS ISK | |
| Spirochaetes | MSFTISYPFK LIIKNKDEAK ALLDTHQYMN EGVKYYLEKL LMFRQEKIFI | (SEQ ID |
| bacterium | GEDETGKRIY IEETEYKKQI EEFYLIKKTE LGRNLTLTLD EFKTLMRELY | NO: 182) |
| GWB1_27_13 | ICLVSSSMEN KKGFPNAQQA SLNIFSPLFD AESKGYILKE ENNNISLIHK | |
| OHD16008.1 | DYGKILLKRL RDNNLIPIFT KFTDIKKITA KLSPTALDRM IFAQAIEKLL | |
| | SYESWCKLMI KERFDKEVKI KELENKCENK QERDKIFEIL EKYEEERQKT | |
| | FEQDSGFAKK GKFYITGRML KGFDEIKEKW LKEKDRSEQN LINILNKYQT | |
| | DNSKLVGDRN LFEFIIKLEN QCLWNGDIDY LKIKRDINKN QIWLDRPEMP | |
| | RFTMPDFKKH PLWYRYEDPS NSNFRNYKIE VVKDENYITI PLITERNNEY | |
| | FEENYTFNLA KLKKLSENIT FIPKSKNKEF EFIDSNDEEE DKKDQKKSKQ | |
| | YIKYCDTAKN TSYGKSGGIR LYFNRNELEN YKDGKKMDSY TVFTLSIRDY | |
| | KSLFAKEKLQ PQIFNTVDNK ITSLKIQKKF GNEEQTNFLS YFTQNQITKK | |
| | DWMDEKTFQN VKELNEGIRV LSVDLGQRFF AAVSCFEIMS EIDNNKLFFN | |
| | LNDQNHKIIR INDKNYYAKH IYSKTIKLSG EDDDLYKERK INKNYKLSYQ | |
| | ERKNKIGIFT RQINKLNQLL KIIRNDEIDK EKFKELIETT KRYVKNTYND | |
| | GIIDWNNVDN KILSYENKED VINLHKELDK KLEIDFKEFI RECRKPIFRS | |
| | GGLSMQRIDF LEKLNKLKRK WVARTQKSAE SIVLTPKFGY KLKEHINELK | |
| | DNRVKQGVNY ILMTALGYIK DNEIKNDSKK KQKEDWVKKN RACQIILMEK | |
| | LTEYTFAEDR PREENSKLRM WSHRQIFNFL QQKASLWGIL VGDVFAPYTS | |
| | KCLSDNNAPG IRCHQVTKKD LIDNSWFLKI VVKDDAFCDL IEINKENVKN | |
| | KSIKINDILP LRGGELFASI KDGKLHIVQA DINASRNIAK RFLSQINPFR | |
| | VVLKKDKDET FHLKNEPNYL KNYYSILNFV PTNEELTFFK VEENKDIKPT | |
| | KRIKMDKHEK ESTDEGDDYS KNQIALFRDD SGIFFDKSLW VDGKIFWSVV | |
| | KNKMTKLLRE RNNKKNGSK | |
| Verrucomicrobiaceae | MPLSRIYQGR TNSLIILTPT PQEPWDHKAL ARFDSPLWRH HALFQDAVNY | (SEQ ID |
| bacterium | YQLCLVALAS SDGTRPLSKL HEQMKASWDE AKTDTEDSWR VRLARRLGIP | NO: 183) |
| UBA2429 | AASLFEAALA KVLEGNEAPE RARELAGELL LDKIEGDIQQ AGRGYWPRFC | |
| GCA_002343505.1 | DPKANPTYDY SATARASASG LTKLAAVIHA ENVTEEALKQ VAAEMDLSWT | |
| | VKLQPDKNFV GAEARARLLE AAHHFIKVAE SPPTKLAEVL ARFPDGLALW | |
| | QALPEKIAAL PEETQVPRNR KASPDLTFAT LLFQHFPSLF TAAVLGLSVG | |
| | KPKSVKAPKV VEKVSARRKA NAVTQAVVIE EPEIDFAELG DDPIKLARGE | |
| | RGFVFPAFTS LSFWAVPGPH VPVWKEFDIA AFKEALKTVN QFKLKTSERN | |
| | ALLAEAQRRL DYMDEKTHDW KTGDSDEPGH IPPRLKSDPN FTLIQALTQD | |
| | EGVSNKATGD QHIPKGVYTG GLRGFYAIKK DWCELWERKA DKSQGTPTEE | |
| | ELISIVTDYQ RDHVYDVGDV GLFRALCEPR FWPLWQPLTD EQEAERIKAG | |
| | RAKDMISAYR VWLELQEDVV RLAQPIRFTP AHAENSRRLF MFSDISGSHG | |
| | AEFGSDGKSL EVSIAYDVDG KLQPVRAKLE FSAPRAARDE LEGLSGGSES | |
| | MRWFQPMMKA LDCPEVEMPA LEKCAVSLMP DVVKKGGGKW VRLLLNFPAT | |
| | LEPEGLIRHI GKQAMWYKQF NGTYKPRTQQ LDTGLHLYWP GLEKAPEAED | |
| | AAAWWNREEI RAKGFSVLSV DLGQRDAGAW ALLESRSDKA FSRNRQPFIE | |
| | LGEAGGKLWS TALLGLGMLR LPGEDARTGA LDDQGKRAVE FHGKAGRNAL | |
| | EAEWQEAREM ALLFGGEEAK SRLGPGFDHL SHSKQNEELL RILSRAQSRL | |
| | ARFHRWSCRI HEKPEATGDD VIDYGQVDEL LTKTAEAMLE NLKALYTNAG | |
| | GILDSKSKQP LTLVGLRKKL EAQKVEPEKI AAVLKPHAEI IFQRLGTLIP | |
| | ELKQHLRVSL ERLANRELPL RHREWVWNEA FEKLEQGNFK KEENPKWIRG | |
| | QRGLSMARIE QIENLRKRFM SLRRQMSLIP GEQVKQGVED KGQRQPEPCE | |
| | DILNKLDRMK QQRVNQTAHL ILAQALGLRL RPHLANDAER EEKDIHGEYE | |
| | LIPGRKPVDF IVMEDLSRYL SSQGRAPSEN GRLMKWCHRA VLAKLKQMCE | |
| | PFGIPVLEVP AAYSSRFCAL TGVPGFRAVE VHDGNAEDFR WKRLIKKAEK | |
| | DKSSKDAEAA AMLFDQLHDL NIEAREARKQ DKKLPLRTLF APVAGGPLFI | |
| | PMVGGGPRQA DMNAAINLGL RAIASPTCLR ARPKIRAELK DGKHQAMLGN | |
| | KLEKAAALTL EPPKEPTKEL AAQKRTNFFL DEKFVGKFDT AHVTTSGKKL | |
| | RLSGGMSLWK AIKDGAWQRV KKINDARIAK WKNNPPPEPD PDDEIQF | |
| Alicyclobacillus | MAVKSIKVKL RLSECPDILA GMWQLHRATN AGVRYYTEWV SLMRQEILYS | (SEQ ID |
| kakegawensis | RGPDGGQQCY MTAEDCQREL LRRLRNRQLH NGRQDQPGTD ADLLAISRRL | NO: 184) |
| WP_067936067.1 | YEILVLQSIG KRGDAQQIAS SFLSPLVDPN SKGGRGEAKS GRKPAWQKMR | |
| | DQGDPRWVAA REKYEQRKAV DPSKEILNSL DALGLRPLFA VFTETYRSGV | |
| | DWKPLGKSQG VRTWDRDMFQ QALERLMSWE SWNRRVGEEY ARLFQQKMKF | |
| | EQEHFAEQSH LVKLARALEA DMRAASQGFE AKRGTAHQIT RRALRGADRV | |
| | FEIWKSIPEE ALFSQYDEVI RQVQAEKRRD FGSHDLFAKL AEPKYQPLWR | |
| | ADETFLTRYA LYNGVLRDLE KARQFATFTL PDACVNPIWT RFESSQGSNL | |
| | HKYEFLFDHL GPGRHAVRFQ RLLVVESEGA KERDSVVVPV APSGQLDKLV | |
| | LREEEKSSVA LHLHDTARPD GFMAEWAGAK LQYERSTLAR KARRDKQGMR | |
| | SWRRQPSMLM SAAQMLEDAK QAGDVYLNIS VRVKSPSEVR GQRRPPYAAL | |
| | FRIDDKQRRV TVNYNKLSAY LEEHPDKQIP GAPGLLSGLR VMSVDLGLRT | |
| | SASISVFRVA KKEEVEALGD GRPPHYYPIH GTDDLVAVHE RSHLIQMPGE | |
| | TETKQLRKLR EERQAVLRPL FAQLALLRLL VRCGAADERI RTRSWQRLTK | |
| | QGREFTKRLT PSWREALELE LTRLEAYCGR VPDDEWSRIV DRTVIALWRR | |
| | MGKQVRDWRK QVKSGAKVKV KGYQLDVVGG NSLAQIDYLE QQYKFLRRWS | |
| | FFARASGLVV RADRESHFAV ALRQHIENAK RDRLKKLADR ILMEALGYVY | |
| | EASGPREGQW TAQHPPCQLI ILEELSAYRF SDDRPPSENS KLMAWGHRGI | |
| | LEELVNQAQV HDVLVGTVYA AFSSRFDART GAPGVRCRRV PARFVGATVD | |
| | DSLPLWLTEF LDKHRLDKNL LRPDDVIPTG EGEFLVSPCG EEAARVRQVH | |
| | ADINAAQNLQ RRLWQNFDIT ELRLRCDVKM GGEGTVLVPR VNNARAKQLF | |
| | GKKVLVSQDG VTFFERSQTG GKPHSEKQTD LTDKELELIA EADEARAKSV | |
| | VLFRDPSGHI GKGHWIRQRE FWSLVKQRIE SHTAERIRVR GVGSSLD | |
| Bacillus sp._ | MAIRSIKLKM KTNSGTDSIY LRKALWRTHQ LINEGIAYYM NLLTLYRQEA | (SEQ ID |
| V3-13 | IGDKTKEAYQ AELINIIRNQ QRNNGSSEEH GSDQEILALL RQLYELIIPS | NO: 185) |
| WP_101661451.1 | SIGESGDANQ LGNKFLYPLV DPNSQSGKGT SNAGRKPRWK RLKEEGNPDW | |
| | ELEKKKDEER KAKDPTVKIF DNLNKYGLLP LFPLFTNIQK DIEWLPLGKR | |
| | QSVRKWDKDM FIQAIERLLS WESWNRRVAD EYKQLKEKTE SYYKEHLTGG | |
| | EEWIEKIRKF EKERNMELEK NAFAPNDGYF ITSRQIRGWD RVYEKWSKLP | |
| | ESASPEELWK VVAEQQNKMS EGFGDPKVFS FLANRENRDI WRGHSERIYH | |
| | IAAYNGLQKK LSRTKEQATF TLPDAIEHPL WIRYESPGGT NLNLFKLEEK | |
| | QKKNYYVTLS KIIWPSEEKW IEKENIEIPL APSIQFNRQI KLKQHVKGKQ | |
| | EISFSDYSSR ISLDGVLGGS RIQFNRKYIK NHKELLGEGD IGPVFFNLVV | |
| | DVAPLQETRN GRLQSPIGKA LKVISSDFSK VIDYKPKELM DWMNTGSASN | |
| | SFGVASLLEG MRVMSIDMGQ RTSASVSIFE VVKELPKDQE QKLFYSINDT | |
| | ELFAIHKRSF LLNLPGEVVT KNNKQQRQER RKKRQFVRSQ IRMLANVLRL | |
| | ETKKTPDERK KAIHKLMEIV QSYDSWTASQ KEVWEKELNL LTNMAAFNDE | |
| | IWKESLVELH HRIEPYVGQI VSKWRKGLSE GRKNLAGISM WNIDELEDTR | |
| | RLLISWSKRS RTPGEANRIE TDEPFGSSLL QHIQNVKDDR LKQMANLIIM | |
| | TALGFKYDKE EKDRYKRWKE TYPACQIILF ENLNRYLFNL DRSRRENSRL | |
| | MKWAHRSIPR TVSMQGEMFG LQVGDVRSEY SSRFHAKTGA PGIRCHALTE | |
| | EDLKAGSNTL KRLIEDGFIN ESELAYLKKG DIIPSQGGEL FVTLSKRYKK | |
| | DSDNNELTVI HADINAAQNL QKRFWQQNSE VYRVPCQLAR MGEDKLYIPK | |
| | SQTETIKKYF GKGSFVKNNT EQEVYKWEKS EKMKIKTDTT FDLQDLDGFE | |
| | DISKTIELAQ EQQKKYLTMF RDPSGYFFNN ETWRPQKEYW SIVNNIIKSC | |
| | LKKKILSNKV EL | |
| Desulfatirhabdium | MPLSNNPPVT QRAYTLRLRG ADPSDLSWRE ALWHTHEAVN KGAKVFGDWL | (SEQ ID |
| butyrativorans | LTLRGGLDHT LADTKVKGGK GKPDRDPTPE ERKARRILLA LSWLSVESKL | NO: 186) |
| WP_028326052.1 | GAPSSYIVAS GDEPAKDRND NVVSALEEIL QSRKVAKSEI DDWKRDCSAS | |
| | LSAAIRDDAV WVNRSKVFDE AVKSVGSSLT REEAWDMLER FFGSRDAYLT | |
| | PMKDPEDKSS ETEQEDKAKD LVQKAGQWLS SRYGTSEGAD FCRMSDIYGK | |
| | IAAWADNASQ GGSSTVDDLV SELRQHFDTK ESKATNGLDW IIGLSSYTGH | |
| | TPNPVHELLR QNTSLNKSHL DDLKKKANTR AESCKSKIGS KGQRPYSDAI | |
| | LNDVESVCGF TYRVDKDGQP VSVADYSKYD VDYKWGTARH YIFAVMLDHA | |
| | ARRISLAHKW IKRAEAERHK FEEDAKRIAN VPARAREWLD SFCKERSVTS | |
| | GAVEPYRIRR RAVDGWKEVV AAWSKSDCKS TEDRIAAARA LQDDSEIDKF | |
| | GDIQLFEALA EDDALCVWHK DGEATNEPDF QPLIDYSLAI EAEFKKRQFK | |
| | VPAYRHPDEL LHPVFCDFGK SRWKINYDVH KNVQAPFYRG LCLTLWTGSE | |
| | IKPVPLCWQS KRLTRDLALG NNHRNDAASA VTRADRLGRA ASNVTKSDMV | |
| | NITGLFEQAD WNGRLQAPRQ QLEAIAVVRD NPRLSEQERN LRMCGMIEHI | |
| | RWLVTFSVKL QPQGPWCAYA EQHGLNTNPQ YWPHADTNRD RKVHARLILP | |
| | RLPGLRVLSV DLGHRYAAAC AVWEAVNTET VKEACQNVGR DMPKEHDLYL | |
| | HIKVKKQGIG KQTEVDKTTI YRRIGADTLP DGRPHPAPWA RLDRQFLIKL | |
| | QGEEKDAREA SNEEIWALHQ MECKLDRTKP LIDRLIASGW GLLKRQMARL | |
| | DALKELGWIP APDSSENLSR EDGEAKDYRE SLAVDDLMFS AVRTLRLALQ | |
| | RHGNRARIAY YLISEVKIRP GGIQEKLDEN GRIDLLQDAL ALWHELFSSP | |
| | GWRDEAAKQL WDSRIATLAG YKAPEENGDN VSDVAYRKKQ QVYREQLRNV | |
| | AKTLSGDVIT CKELSDAWKE RWEDEDQRWK KLLRWFKDWV LPSGTQANNA | |
| | TIRNVGGLSL SRLATITEFR RKVQVGFFTR LRPDGTRHEI GEQFGQKTLD | |
| | ALELLREQRV KQLASRIAEA ALGIGSEGGK GWDGGKRPRQ RINDSRFAPC | |
| | HAVVIENLAN YRPDETRTRL ENRRLMTWSA SKVHKYLSEA CQLNGLYLCT | |
| | VSAWYTSRQD SRTGAPGIRC QDVSVREFMQ SPFWRKQVKQ AEAKHDENKG | |
| | DARERFLCEL NKTWKAKTPA EWKKAGFVRI PLRGGEIFVS ADSKSPSAKG | |
| | IHADLNAAAN IGLRALTDPD WPGKWWYVPC DPVSFESKMD YVKGCAAVKV | |
| | GQPLRQPAQT NADGAASKIR KGKKNRTAGT SKEKVYLWRD ISAFPLESNE | |
| | IGEWKETSAY QNDVQYRVIR MLKEHIKSLD NRTGDNVEG | |
| Desulfonatronum | MVLGRKDDTA ELRRALWITH EHVNLAVAEV ERVLLRCRGR SYWTLDRRGD | (SEQ ID |
| thiodismutans | PVHVPESQVA EDALAMAREA QRRNGWPVVG EDEEILLALR YLYEQIVPSC | NO: 187) |
| WP_031386437.1 | LLDDLGKPLK GDAQKIGTNY AGPLFDSDTC RRDEGKDVAC CGPFHEVAGK | |
| | YLGALPEWAT PISKQEFDGK DASHLRFKAT GGDDAFFRVS IEKANAWYED | |
| | PANQDALKNK AYNKDDWKKE KDKGISSWAV KYIQKQLQLG QDPRTEVRRK | |
| | LWLELGLLPL FIPVFDKTMV GNLWNRLAVR LALAHLLSWE SWNHRAVQDQ | |
| | ALARAKRDEL AALFLGMEDG FAGLREYELR RNESIKQHAF EPVDRPYVVS | |
| | GRALRSWTRV REEWLRHGDT QESRKNICNR LQDRLRGKFG DPDVFHWLAE | |
| | DGQEALWKER DCVTSFSLLN DADGLLEKRK GYALMTFADA RLHPRWAMYE | |
| | APGGSNLRTY QIRKTENGLW ADVVLLSPRN ESAAVEEKTF NVRLAPSGQL | |
| | SNVSFDQIQK GSKMVGRCRY QSANQQFEGL LGGAEILFDR KRIANEQHGA | |
| | TDLASKPGHV WFKLTLDVRP QAPQGWLDGK GRPALPPEAK HFKTALSNKS | |
| | KFADQVRPGL RVLSVDLGVR SFAACSVFEL VRGGPDQGTY FPAADGRTVD | |
| | DPEKLWAKHE RSFKITLPGE NPSRKEEIAR RAAMEELRSL NGDIRRLKAI | |
| | LRLSVLQEDD PRTEHLRLFM EAIVDDPAKS ALNAELFKGF GDDRFRSTPD | |
| | LWKQHCHFFH DKAEKVVAER FSRWRTETRP KSSSWQDWRE RRGYAGGKSY | |
| | WAVTYLEAVR GLILRWNMRG RTYGEVNRQD KKQFGTVASA LLHHINQLKE | |
| | DRIKTGADMI IQAARGFVPR KNGAGWVQVH EPCRLILFED LARYRFRTDR | |
| | SRRENSRLMR WSHREIVNEV GMQGELYGLH VDTTEAGFSS RYLASSGAPG | |
| | VRCRHLVEED FHDGLPGMHL VGELDWLLPK DKDRTANEAR RLLGGMVRPG | |
| | MLVPWDGGEL FATLNAASQL HVIHADINAA QNLQRRFWGR CGEAIRIVCN | |
| | QLSVDGSTRY EMAKAPKARL LGALQQLKNG DAPFHLTSIP NSQKPENSYV | |
| | MTPTNAGKKY RAGPGEKSSG EEDELALDIV EQAEELAQGR KTFFRDPSGV | |
| | FFAPDRWLPS EIYWSRIRRR IWQVTLERNS SGRQERAEMD EMPY | |
| Lentisphaeria | MAVELNRIYQ GRVNHVYIFD ENQNQVSVDN GDDLLFVHHE LYQDAINYYL | (SEQ ID |
| bacterium | VALAAMALDS KDSLFGKFKM QIRAVWNDFY RNGQLRPGLK HSLIRSLGHA | NO: 188) |
| DCFZ01000012.1 | AELNTSNGAD IAMNLILEDG GIPSEILNAA LEHLAEKCTG DVSQLGKTFF | |
| | PRFCDTAYHG NWDVDAKSFS EKKGRQRLVD ALYSLHPVQA VQELAPEIEI | |
| | GWGGVKTQTG KFFTGDEAKA SLKKAISYFL QDTGKNSPEL QEYFSVAGKQ | |
| | PLEQYLGKID TFPEISFGRI SSHQNINISN AMWILKFFPD QYSVDLIKNL | |
| | IPNKKYEIGI APQWGDDPVK LSRGKRGYTF RAFTDLAMWE KNWKVFDRAA | |
| | FSDALKTINQ FRNKTQERND QLKRYCAALN WMDGESSDKK PPVEPADADA | |
| | VDEAATSVLP ILAGDKRWNA LLQLQKELGI CNDFTENELM DYGLSLRTIR | |
| | GYQKLRSMML EKEEKMRAKT ADDEEISQAL QEIIIKFQSS HRDTIGSVSL | |
| | FLKLAEPKYF CVWHDADKNQ NFASVDMVAD AVRYYSYQEE KARLEEPIQI | |
| | TPADARYSRR VSDLYALVYK NAKECKTGYG LRPDGNFVFE IAQKNAKGYA | |
| | PAKVVLAFSA PRLKRDGLID KEFSAYYPPV LQAFLREEEA PKQSFKTTAV | |
| | ILMPDWDKNG KRRILLNFPI KLDVSAIHQK TDHRFENQFY FANNTNTCLL | |
| | WPSYQYKKPV TWYQGKKPFD VVAVDLGQRS AGAVSRITVS TEKREHSVAI | |
| | GEAGGTQWYA YRKFSGLLRL PGEDATVIRD GQRTEELSGN AGRLSTEEET | |
| | VQACVLCKML IGDATLLGGS DEKTIRSFPK QNDKLLIAFR RATGRMKQLQ | |
| | RWLWMLNENG LCDKAKTEIS NSDWLVNKNI DNVLKEEKQH REMLPAILLQ | |
| | IADRVLPLRG RKWDWVLNPQ SNSFVLQQTA HGSGDPHKKI CGQRGLSFAR | |
| | IEQLESLRMR CQALNRILMR KTGEKPATLA EMRNNPIPDC CPDILMRLDA | |
| | MKEQRINQTA NLILAQALGL RHCLHSESAT KRKENGMHGE YEKIPGVEPA | |
| | AFVVLEDLSR YRFSQDRSSY ENSRLMKWSH RKILEKLALL CEVFNVPILQ | |
| | VGAAYSSKFS ANAIPGFRAE ECSIDQLSFY PWRELKDSRE KALVEQIRKI | |
| | GHRLLTFDAK ATIIMPRNGG PVFIPFVPSD SKDTLIQADI NASFNIGLRG | |
| | VADATNLLCN NRVSCDRKKD CWQVKRSSNF SKMVYPEKLS LSFDPIKKQE | |
| | GAGGNFFVLG CSERILTGTS EKSPVFTSSE MAKKYPNLMF GSALWRNEIL | |
| | KLERCCKINQ SRLDKFIAKK EVQNEL | |
| Laceyella sediminis | MSIRSFKLKI KTKSGVNAEE LRRGLWRTHQ LINDGIAYYM NWLVLLRQED | (SEQ ID |
| WP_106341859.1 | LFIRNEETNE IEKRSKEEIQ GELLERVHKQ QQRNQWSGEV DDQTLLQTLR | NO: 189) |
| | HLYEEIVPSV IGKSGNASLK ARFFLGPLVD PNNKTTKDVS KSGPTPKWKK | |
| | MKDAGDPNWV QEYEKYMAER QTLVRLEEMG LIPLFPMYTD EVGDIHWLPQ | |
| | ASGYTRTWDR DMFQQAIERL LSWESWNRRV RERRAQFEKK THDFASRFSE | |
| | SDVQWMNKLR EYEAQQEKSL EENAFAPNEP YALTKKALRG WERVYHSWMR | |
| | LDSAASEEAY WQEVATCQTA MRGEFGDPAI YQFLAQKENH DIWRGYPERV | |
| | IDFAELNHLQ RELRRAKEDA TFTLPDSVDH PLWVRYEAPG GTNIHGYDLV | |
| | QDTKRNLTLI LDKFILPDEN GSWHEVKKVP FSLAKSKQFH RQVWLQEEQK | |
| | QKKREVVFYD YSTNLPHLGT LAGAKLQWDR NFLNKRTQQQ IEETGEIGKV | |
| | FFNISVDVRP AVEVKNGRLQ NGLGKALTVL THPDGTKIVT GWKAEQLEKW | |
| | VGESGRVSSL GLDSLSEGLR VMSIDLGQRT SATVSVFEIT KEAPDNPYKF | |
| | FYQLEGTELF AVHQRSFLLA LPGENPPQKI KQMREIRWKE RNRIKQQVDQ | |
| | LSAILRLHKK VNEDERIQAI DKLLQKVASW QLNEEIATAW NQALSQLYSK | |
| | AKENDLQWNQ AIKNAHHQLE PVVGKQISLW RKDLSTGRQG IAGLSLWSIE | |
| | ELEATKKLLT RWSKRSREPG VVKRIERFET FAKQIQHHIN QVKENRLKQL | |
| | ANLIVMTALG YKYDQEQKKW IEVYPACQVV LFENLRSYRF SYERSRRENK | |
| | KLMEWSHRSI PKLVQMQGEL FGLQVADVYA AYSSRYHGRT GAPGIRCHAL | |
| | TEADLRNETN IIHELIEAGF IKEEHRPYLQ QGDLVPWSGG ELFATLQKPY | |
| | DNPRILTLHA DINAAQNIQK RFWHPSMWFR VNCESVMEGE IVTYVPKNKT | |
| | VHKKQGKTFR FVKVEGSDVY EWAKWSKNRN KNTFSSITER KPPSSMILFR | |
| | DPSGTFFKEQ EWVEQKTFWG KVQSMIQAYM KKTIVQRMEE | |
| Methylobacterium | MYEAIVLADD ANAQLANAFL GPLTDPNSAG FLEAFNKVDR PAPSWLDQVP | (SEQ ID |
| nodulans (long | ASDPIDPAVL AEANAWLDTD AGRAWLVDTG APPRWRSLAA KQDPIWPREF | NO: 190) |
| form) | ARKLGELRKE AASGTSAIIK ALKRDFGVLP LFQPSLAPRI LGSRSSLTPW | |
| | DRLAFRLAVG HLLSWESWCT RARDEHTARV QRLEQFSSAH LKGDLATKVS | |
| | TLREYERARK EQIAQLGLPM GERDFLITVR MTRGWDDLRE KWRRSGDKGQ | |
| | EALHAIIATE QTRKRGRFGD PDLFRWLARP ENHHVWADGH ADAVGVLARV | |
| | NAMERLVERS RDTALMTLPD PVAHPRSAQW EAEGGSNLRN YQLEAVGGEL | |
| | QITLPLLKAA DDGRCIDTPL SFSLAPSDQL QGVVLTKQDK QQKITYCTNM | |
| | NEVFEAKLGS ADLLLNWDHL RGRIRDRVDA GDIGSAFLKL ALDVAHVLPD | |
| | GVDDQLARAA FHFQSAKGAK SKHADSVQAG LRVLSIDLGV RSFATCSVFE | |
| | LKDTAPTTGV AFPLAEFRLW AVHERSFTLE LPGENVGAAG QQWRAQADAE | |
| | LRQLRGGLNR HRQLLRAATV QKGERDAYLT DLREAWSAKE LWPFEASLLS | |
| | ELERCSTVAD PLWQDTCKRA ARLYRTEFGA VVSEWRSRTR SREDRKYAGK | |
| | SMWSVQHLTD VRRFLQSWSL AGRASGDIRR LDRERGGVFA KDLLDHIDAL | |
| | KDDRLKTGAD LIVQAARGFQ RNEFGYWVQK HAPCHVILFE DLSRYRMRTD | |
| | RPRRENSQLM QWAHRGVPDM VGMQGEIYGI QDRRDPDSAR KHARQPLAAF | |
| | CLDTPAAFSS RYHASTMTPG IRCHPLRKRE FEDQGFLELL KRENEGLDLN | |
| | GYKPGDLVPL PGGEVFVCLN ANGLSRIHAD INAAQNLQRR FWTQHGDAFR | |
| | LPCGKSAVQG QIRWAPLSMG KRQAGALGGF GYLEPTGHDS GSCQWRKTTE | |
| | AEWRRLSGAQ KDRDEAAAAE DEELQGLEEE LLERSGERVV FFRDPSGVVL | |
| | PTDLWFPSAA FWSIVRAKTV GRLRSHLDAQ AEASYAVAAG L | |
| Opitutaceae | MSLNRIYQGR VAAVETGTAL AKGNVEWMPA AGGDEVLWQH HELFQAAINY | (SEQ ID |
| bacterium | YLVALLALAD KNNPVLGPLI SQMDNPQSPY HVWGSFRRQG RQRTGLSQAV | NO: 191) |
| WP_009513281.1 | APYITPGNNA PTLDEVFRSI LAGNPTDRAT LDAALMQLLK ACDGAGAIQQ | |
| | EGRSYWPKFC DPDSTANFAG DPAMLRREQH RLLLPQVLHD PAITHDSPAL | |
| | GSFDTYSIAT PDTRTPQLTG PKARARLEQA ITLWRVRLPE SAADFDRLAS | |
| | SLKKIPDDDS RLNLQGYVGS SAKGEVQARL FALLLFRHLE RSSFTLGLLR | |
| | SATPPPKNAE TPPPAGVPLP AASAADPVRI ARGKRSFVFR AFTSLPCWHG | |
| | GDNIHPTWKS FDIAAFKYAL TVINQIEEKT KERQKECAEL ETDFDYMHGR | |
| | LAKIPVKYTT GEAEPPPILA NDLRIPLLRE LLQNIKVDTA LTDGEAVSYG | |
| | LQRRTIRGFR ELRRIWRGHA PAGTVFSSEL KEKLAGELRQ FQTDNSTTIG | |
| | SVQLFNELIQ NPKYWPIWQA PDVETARQWA DAGFADDPLA ALVQEAELQE | |
| | DIDALKAPVK LTPADPEYSR RQYDFNAVSK FGAGSRSANR HEPGQTERGH | |
| | NTFTTEIAAR NAADGNRWRA THVRIHYSAP RLLRDGLRRP DTDGNEALEA | |
| | VPWLQPMMEA LAPLPTLPQD LTGMPVFLMP DVTLSGERRI LLNLPVTLEP | |
| | AALVEQLGNA GRWQNQFFGS REDPFALRWP ADGAVKTAKG KTHIPWHQDR | |
| | DHFTVLGVDL GTRDAGALAL LNVTAQKPAK PVHRIIGEAD GRTWYASLAD | |
| | ARMIRLPGED ARLFVRGKLV QEPYGERGRN ASLLEWEDAR NIILRLGQNP | |
| | DELLGADPRR HSYPEINDKL LVALRRAQAR LARLQNRSWR LRDLAESDKA | |
| | LDEIHAERAG EKPSPLPPLA RDDAIKSTDE ALLSQRDIIR RSFVQIANLI | |
| | LPLRGRRWEW RPHVEVPDCH ILAQSDPGTD DTKRIVAGQR GISHERIEQI | |
| | EELRRRCQSL NRALRHKPGE RPVLGRPAKG EEIADPCPAL LEKINRLRDQ | |
| | RVDQTAHAIL AAALGVRLRA PSKDRAERRH RDIHGEYERF RAPADFVVIE | |
| | NLSRYLSSQD RARSENTRLM QWCHRQIVQK LRQLCETYGI PVLAVPAAYS | |
| | SRFSSRDGSA GFRAVHLTPD HRHRMPWSRI LARLKAHEED GKRLEKTVLD | |
| | EARAVRGLFD RLDRFNAGHV PGKPWRTLLA PLPGGPVFVP LGDATPMQAD | |
| | LNAAINIALR GIAAPDRHDI HHRLRAENKK RILSLRLGTQ REKARWPGGA | |
| | PAVTLSTPNN GASPEDSDAL PERVSNLFVD IAGVANFERV TIEGVSQKFA | |
| | TGRGLWASVK QRAWNRVARL NETVTDNNRN EEEDDIPM | |
| Thermomonas | MSEKTTQRAY TLRLNRASGE CAVCQNNSCD CWHDALWATH KAVNRGAKAF | (SEQ ID |
| hydrothermalis | GDWLLTLRGG LCHTLVEMEV PAKGNNPPQR PTDQERRDRR VLLALSWLSV | NO: 192) |
| WP_072754838.1 | EDEHGAPKEF IVATGRDSAD DRAKKVEEKL REILEKRDFQ EHEIDAWLQD | |
| | CGPSLKAHIR EDAVWVNRRA LFDAAVERIK TLTWEEAWDF LEPFFGTQYF | |
| | AGIGDGKDKD DAEGPARQGE KAKDLVQKAG QWLSARFGIG TGADFMSMAE | |
| | AYEKIAKWAS QAQNGDNGKA TIEKLACALR PSEPPTLDTV LKCISGPGHK | |
| | SATREYLKTL DKKSTVTQED LNQLRKLADE DARNCRKKVG KKGKKPWADE | |
| | VLKDVENSCE LTYLQDNSPA RHREFSVMLD HAARRVSMAH SWIKKAEQRR | |
| | RQFESDAQKL KNLQERAPSA VEWLDRFCES RSMTTGANTG SGYRIRKRAI | |
| | EGWSYVVQAW AEASCDTEDK RIAAARKVQA DPEIEKFGDI QLFEALAADE | |
| | AICVWRDQEG TQNPSILIDY VTGKTAEHNQ KRFKVPAYRH PDELRHPVFC | |
| | DFGNSRWSIQ FAIHKEIRDR DKGAKQDTRQ LQNRHGLKMR LWNGRSMTDV | |
| | NLHWSSKRLT ADLALDQNPN PNPTEVTRAD RLGRAASSAF DHVKIKNVFN | |
| | EKEWNGRLQA PRAELDRIAK LEEQGKTEQA EKLRKRLRWY VSFSPCLSPS | |
| | GPFIVYAGQH NIQPKRSGQY APHAQANKGR ARLAQLILSR LPDLRILSVD | |
| | LGHRFAAACA VWETLSSDAF RREIQGLNVL AGGSGEGDLF LHVEMTGDDG | |
| | KRRTVVYRRI GPDQLLDNTP HPAPWARLDR QFLIKLQGED EGVREASNEE | |
| | LWTVHKLEVE VGRTVPLIDR MVRSGFGKTE KQKERLKKLR ELGWISAMPN | |
| | EPSAETDEKE GEIRSISRSV DELMSSALGT LRLALKRHGN RARIAFAMTA | |
| | DYKPMPGGQK YYFHEAKEAS KNDDETKRRD NQIEFLQDAL SLWHDLFSSP | |
| | DWEDNEAKKL WQNHIATLPN YQTPEEISAE LKRVERNKKR KENRDKLRTA | |
| | AKALAENDQL RQHLHDTWKE RWESDDQQWK ERLRSLKDWI FPRGKAEDNP | |
| | SIRHVGGLSI TRINTISGLY QILKAFKMRP EPDDLRKNIP QKGDDELENF | |
| | NRRLLEARDR LREQRVKQLA SRIIEAALGV GRIKIPKNGK LPKRPRTTVD | |
| | TPCHAVVIES LKTYRPDDLR TRRENRQLMQ WSSAKVRKYL KEGCELYGLH | |
| | FLEVPANYTS RQCSRTGLPG IRCDDVPTGD FLKAPWWRRA INTAREKNGG | |
| | DAKDRFLVDL YDHLNNLQSK GEALPATVRV PRQGGNLFIA GAQLDDTNKE | |
| | RRAIQADLNA AANIGLRALL DPDWRGRWWY VPCKDGTSEP ALDRIEGSTA | |
| | FNDVRSLPTG DNSSRRAPRE IENLWRDPSG DSLESGTWSP TRAYWDTVQS | |
| | RVIELLRRHA GLPTS | |
| Methylobacterium | MYEAIVLADD ANAQLANAFL GPLTDPNSAG FLEAFNKVDR PAPSWLDQVP | (SEQ ID |
| nodulans | ASDPIDPAVL AEANAWLDTD AGRAWLVDTG APPRWRSLAA KQDPIWPREF | NO: 193) |
| WP_043747912.1 | ARKLGELRKE AASGTSAIIK ALKRDFGVLP LFQPSLAPRI LGSRSSLTPW | |
| | DRLAFRLAVG HLLSWESWCT RARDEHTARV QRLEQFSSAH LKGDLATKVS | |
| | TLREYERARK EQIAQLGLPM GERDFLITVR MTRGWDDLRE KWRRSGDKGQ | |
| | EALHAIIATE QTRKRGRFGD PDLFRWLARP ENHHVWADGH ADAVGVLARV | |
| | NAMERLVERS RDTALMTLPD PVAHPRSAQW EAEGGSNLRN YQLEAVGGEL | |
| | QITLPLLKAA DDGRCIDTPL | |
| Chloracidobacterium | MPQQAKPPVT QRAYTLRLRG ADSNDPSWRD ALWQTHEAVN RGAQAFGDWL | (SEQ ID |
| thermophilum | LTLRGGLDHT LADTPVKGGK GKPDPDPTDE ERKARRILLA LSWLSVESKL | NO: 194) |
| WP_058868187.1 | GAPAGLIIAF GTEAAEERNR KVVAALEEIL KSRGVDQNEI NAWKKDCSAS | |
| | LSAAIRDDAV WVNRSKAFDE AVESIGSSGS SGSSLTREEP WDMLERFFGS | |
| | RDAYLAPAKG SEDESSEAKQ EDQAKDLVQK AGQWLSSRFG TGKGADFRRM | |
| | ATVYEAIAKW DGKASLEMAG DKAIADLATA LSEFNPASND LQGVLGLISG | |
| | PGYKSATRNF LNQLAAQTTV TQQDFVSLKD KANNDAQECK QNTGSKGQRP | |
| | YSNSILEKVE SVCGFTYLQD GGPARHSEFA VILDHAARRV SLAHTWIKLA | |
| | EAERRKFEED AKKIDQVPEA AKDWLDRFCL ERSGVSGALE PYRIRRRAVD | |
| | GWKEVVAEWS KSDCKTVEDR IAAARALQDD PEIDKFGDIQ LFEALAEDDA | |
| | VCVWHKDGDA AKAPDPQPLI DYALAAEAEF KKRHFKVPAY RHPDALLHPI | |
| | FCDFGKSRWD ICFDVHKNMQ TPFPRALCLT LWTGSEMKRI PLCWQSKRLA | |
| | RDLALGNNTG DAGASEVTRA DRLGRAASRA ASNVTKSDVV NIAGLFEQAD | |
| | WNGRLQAPRQ QLEAIARYVE KHDWDQKAEK MRNAIQWLVT FSARLQPQGP | |
| | WCAYAKIHGL KEDPQYWPHA DTNKNRKGHA RLILSRLPGL RVLAVDLGHR | |
| | YAAACAVWEA LSTEAFQREI KGRTILRGRT DGNALYCHTR HKANGKERVT | |
| | IYRRIGADTL PDGKPHPAPW ARLDRQFLIK LQGEEEGVRE ASNEEIWAVH | |
| | QLEAALGRPV SLIDRIVASG WGGSDKQKAR LEGLKQLGWD PADKPSLSVD | |
| | ELMSSAVRTM RLALKRHGDR ARIAHYLITD EKTTPGGIKE TLDEKGRIDL | |
| | LQDALVLWHD LFSSRGWRDD TAKQLWNAHV AKLHGYKAPE EPGEDSSGAE | |
| | RKKKQRENRE KLYDVAKALA QDVTLREALH DAWKKRWEND DERWKKQLRW | |
| | FKDWVFPRGN HASDPTIRKR QLINPSGGNG RRGNHASDPT IRKRQLINPS | |
| | GGNGRRGNHA SDPTIRKVGG LSLPRLATLT EFRRKVQVGF FTRLKPDGTR | |
| | AETKEQFGQS ALDALEHLRE QRVKQLASRI AEAALGVGRV RRPVEGKDPK | |
| | RPDVRVDEPC HAIVIEDLTH YRPEETRTRR ENRQLMTWSS SKVKKYLAEA | |
| | CQLHGLHLRE VSASYTSRQD SRTGAPGVRC QDVPVKEFMR SPFWRKQVKQ | |
| | AEAKQAANKG DARERLLCDL NARWKDRTAA DWEKAGAVRI PLQGGEIFVS | |
| | ADANSPAAKG IQADLNAAAN IGLRALTDPD WAGKWWYVPC DPASFRPVRD | |
| | KVDGSAVVNP DQPLRQSAQA QSGDAAKDKN GNKGAGKSKE VVNLWRDISS | |
| | SPLECIEFGE WKEYAAYQNE VQCRVIRILK EQIKGRDKQP HEGSKEDDIP | |
| | L | |
| Desulfovibrio | MPTRTINLKL VLGKNPENAT LRRALFSTHR LVNQATKRIE EFLLLCRGEA | (SEQ ID |
| inopinatus | YRTVDNEGKE AEIPRHAVQE EALAFAKAAQ RHNGCISTYE DQEILDVLRQ | NO: 195) |
| WP_027186183.1 | LYERLVPSVN ENNEAGDAQA ANAWVSPLMS AESEGGLSVY DKVLDPPPVW | |
| | MKLKEEKAPG WEAASQIWIQ SDEGQSLLNK PGSPPRWIRK LRSGQPWQDD | |
| | FVSDQKKKQD ELTKGNAPLI KQLKEMGLLP LVNPFFRHLL DPEGKGVSPW | |
| | DRLAVRAAVA HFISWESWNH RTRAEYNSLK LRRDEFEAAS DEFKDDFTLL | |
| | RQYEAKRHST LKSIALADDS NPYRIGVRSL RAWNRVREEW IDKGATEEQR | |
| | VTILSKLQTQ LRGKFGDPDL FNWLAQDRHV HLWSPRDSVT PLVRINAVDK | |
| | VLRRRKPYAL MTFAHPRFHP RWILYEAPGG SNLRQYALDC TENALHITLP | |
| | LLVDDAHGTW IEKKIRVPLA PSGQIQDLTL EKLEKKKNRL YYRSGFQQFA | |
| | GLAGGAEVLF HRPYMEHDER SEESLLERPG AVWFKLTLDV ATQAPPNWLD | |
| | GKGRVRTPPE VHHFKTALSN KSKHTRTLQP GLRVLSVDLG MRTFASCSVF | |
| | ELIEGKPETG RAFPVADERS MDSPNKLWAK HERSFKLTLP GETPSRKEEE | |
| | ERSIARAEIY ALKRDIQRLK SLLRLGEEDN DNRRDALLEQ FFKGWGEEDV | |
| | VPGQAFPRSL FQGLGAAPFR STPELWRQHC QTYYDKAEAC LAKHISDWRK | |
| | RTRPRPTSRE MWYKTRSYHG GKSIWMLEYL DAVRKLLLSW SLRGRTYGAI | |
| | NRQDTARFGS LASRLLHHIN SLKEDRIKTG ADSIVQAARG YIPLPHGKGW | |
| | EQRYEPCQLI LFEDLARYRF RVDRPRRENS QLMQWNHRAI VAETTMQAEL | |
| | YGQIVENTAA GFSSRFHAAT GAPGVRCRFL LERDFDNDLP KPYLLRELSW | |
| | MLGNTKVESE EEKLRLLSEK IRPGSLVPWD GGEQFATLHP KRQTLCVIHA | |
| | DMNAAQNLQR RFFGRCGEAF RLVCQPHGDD VLRLASTPGA RLLGALQQLE | |
| | NGQGAFELVR DMGSTSQMNR FVMKSLGKKK IKPLQDNNGD DELEDVLSVL | |
| | PEEDDTGRIT VFRDSSGIFF PCNVWIPAKQ FWPAVRAMIW KVMASHSLG | |
| Desulfonatronum | MVLGRKDDTA ELRRALWITH EHVNLAVAEV ERVLLRCRGR SYWTLDRRGD | (SEQ ID |
| thiodismutans | PVHVPESQVA EDALAMAREA QRRNGWPVVG EDEEILLALR YLYEQIVPSC | NO: 187) |
| WP_031386437.1 | LLDDLGKPLK GDAQKIGTNY AGPLFDSDTC RRDEGKDVAC CGPFHEVAGK | |
| | YLGALPEWAT PISKQEFDGK DASHLRFKAT GGDDAFFRVS IEKANAWYED | |
| | PANQDALKNK AYNKDDWKKE KDKGISSWAV KYIQKQLQLG QDPRTEVRRK | |
| | LWLELGLLPL FIPVFDKTMV GNLWNRLAVR LALAHLLSWE SWNHRAVQDQ | |
| | ALARAKRDEL AALFLGMEDG FAGLREYELR RNESIKQHAF EPVDRPYVVS | |
| | GRALRSWTRV REEWLRHGDT QESRKNICNR LQDRLRGKFG DPDVFHWLAE | |
| | DGQEALWKER DCVTSFSLLN DADGLLEKRK GYALMTFADA RLHPRWAMYE | |
| | APGGSNLRTY QIRKTENGLW ADVVLLSPRN ESAAVEEKTF NVRLAPSGQL | |
| | SNVSFDQIQK GSKMVGRCRY QSANQQFEGL LGGAEILFDR KRIANEQHGA | |
| | TDLASKPGHV WFKLTLDVRP QAPQGWLDGK GRPALPPEAK HFKTALSNKS | |
| | KFADQVRPGL RVLSVDLGVR SFAACSVFEL VRGGPDQGTY FPAADGRTVD | |
| | DPEKLWAKHE RSFKITLPGE NPSRKEEIAR RAAMEELRSL NGDIRRLKAI | |
| | LRLSVLQEDD PRTEHLRLFM EAIVDDPAKS ALNAELFKGF GDDRFRSTPD | |
| | LWKQHCHFFH DKAEKVVAER FSRWRTETRP KSSSWQDWRE RRGYAGGKSY | |
| | WAVTYLEAVR GLILRWNMRG RTYGEVNRQD KKQFGTVASA LLHHINQLKE | |
| | DRIKTGADMI IQAARGFVPR KNGAGWVQVH EPCRLILFED LARYRFRTDR | |
| | SRRENSRLMR WSHREIVNEV GMQGELYGLH VDTTEAGFSS RYLASSGAPG | |
| | VRCRHLVEED FHDGLPGMHL VGELDWLLPK DKDRTANEAR RLLGGMVRPG | |
| | MLVPWDGGEL FATLNAASQL HVIHADINAA QNLQRRFWGR CGEAIRIVCN | |
| | QLSVDGSTRY EMAKAPKARL LGALQQLKNG DAPFHLTSIP NSQKPENSYV | |
| | MTPTNAGKKY RAGPGEKSSG EEDELALDIV EQAEELAQGR KTFFRDPSGV | |
| | FFAPDRWLPS EIYWSRIRRR IWQVTLERNS SGRQERAEMD EMPY | |
| Tuberibacillus | MATKSFILKM KTKNNPQLRL SLWKTHELFN FGVAYYMDLL SLFRQKDLYM | (SEQ ID |
| calidus | HNDEDPDHPV VLKKEEIQER LWMKVRETQQ KNGFHGEVSK DEVLETLRAL | NO: 196) |
| WP_027726362.1 | YEELVPSAVG KSGEANQISN KYLYPLTDPA SQSGKGTANS GRKPRWKKLK | |
| | EAGDPSWKDA YEKWEKERQE DPKLKILAAL QSFGLIPLFR PFTENDHKAV | |
| | ISVKWMPKSK NQSVRKFDKD MFNQAIERFL SWESWNEKVA EDYEKTVSIY | |
| | ESLQKELKGI STKAFEIMER VEKAYEAHLR EITFSNSTYR IGNRAIRGWT | |
| | EIVKKWMKLD PSAPQGNYLD VVKDYQRRHP RESGDFKLFE LLSRPENQAA | |
| | WREYPEFLPL YVKYRHAEQR MKTAKKQATF TLCDPIRHPL WVRYEERSGT | |
| | NLNKYRLIMN EKEKVVQFDR LICLNADGHY EEQEDVTVPL APSQQFDDQI | |
| | KFSSEDTGKG KHNFSYYHKG INYELKGTLG GARIQFDREH LLRRQGVKAG | |
| | NVGRIFLNVT LNIEPMQPFS RSGNLQTSVG KALKVYVDGY PKVVNFKPKE | |
| | LTEHIKESEK NTLTLGVESL PTGLRVMSVD LGQRQAAAIS IFEVVSEKPD | |
| | DNKLFYPVKD TDLFAVHRTS FNIKLPGEKR TERRMLEQQK RDQAIRDLSR | |
| | KLKFLKNVLN MQKLEKTDER EKRVNRWIKD REREEENPVY VQEFEMISKV | |
| | LYSPHSVWVD QLKSIHRKLE EQLGKEISKW RQSISQGRQG VYGISLKNIE | |
| | DIEKTRRLLF RWSMRPENPG EVKQLQPGER FAIDQQNHLN HLKDDRIKKL | |
| | ANQIVMTALG YRYDGKRKKW IAKHPACQLV LFEDLSRYAF YDERSRLENR | |
| | NLMRWSRREI PKQVAQIGGL YGLLVGEVGA QYSSRFHAKS GAPGIRCRVV | |
| | KEHELYITEG GQKVRNQKFL DSLVENNIIE PDDARRLEPG DLIRDQGGDK | |
| | FATLDERGEL VITHADINAA QNLQKRFWTR THGLYRIRCE SREIKDAVVL | |
| | VPSDKDQKEK MENLFGIGYL QPFKQENDVY KWVKGEKIKG KKTSSQSDDK | |
| | ELVSEILQEA SVMADELKGN RKTLFRDPSG YVFPKDRWYT GGRYFGTLEH | |
| | LLKRKLAERR LFDGGSSRRG LFNGTDSNTN VE | |
| Bacillus | MATRSFILKI EPNEEVKKGL WKTHEVLNHG IAYYMNILKL IRQEAIYEHH | (SEQ ID |
| thermoamylovorans | EQDPKNPKKV SKAEIQAELW DFVLKMQKCN SFTHEVDKDV VFNILRELYE | NO: 197) |
| WP_041902512.1 | ELVPSSVEKK GEANQLSNKF LYPLVDPNSQ SGKGTASSGR KPRWYNLKIA | |
| | GDPSWEEEKK KWEEDKKKDP LAKILGKLAE YGLIPLFIPF TDSNEPIVKE | |
| | IKWMEKSRNQ SVRRLDKDMF IQALERFLSW ESWNLKVKEE YEKVEKEHKT | |
| | LEERIKEDIQ AFKSLEQYEK ERQEQLLRDT LNTNEYRLSK RGLRGWREII | |
| | QKWLKMDENE PSEKYLEVFK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDFPKFVNF KPKELTEWIK | |
| | DSKGKKLKSG IESLEIGLRV MSIDLGQRQA AAASIFEVVD QKPDIEGKLF | |
| | FPIKGTELYA VHRASFNIKL PGETLVKSRE VLRKAREDNL KLMNQKLNFL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYEERS RFENSKLMKW | |
| | SRREIPRQVA LQGEIYGLQV GEVGAQFSSR FHAKTGSPGI RCSVVTKEKL | |
| | QDNRFFKNLQ REGRLTLDKI AVLKEGDLYP DKGGEKFISL SKDRKLVTTH | |
| | ADINAAQNLQ KRFWTRTHGF YKVYCKAYQV DGQTVYIPES KDQKQKIIEE | |
| | FGEGYFILKD GVYEWGNAGK LKIKKGSSKQ SSSELVDSDI LKDSFDLASE | |
| | LKGEKLMLYR DPSGNVFPSD KWMAAGVFFG KLERILISKL TNQYSISTIE | |
| | DDSSKQSM | |
| Bacillus sp. NSP2.1 | MAIRSIKLKL KTHTGPEAQN LRKGIWRTHR LLNEGVAYYM KMLLLFRQES | (SEQ ID |
| WP_026557978.1 | TGERPKEELQ EELICHIREQ QQRNQADKNT QALPLDKALE ALRQLYELLV | NO: 198) |
| | PSSVGQSGDA QIISRKFLSP LVDPNSEGGK GTSKAGAKPT WQKKKEANDP | |
| | TWEQDYEKWK KRREEDPTAS VITTLEEYGI RPIFPLYTNT VTDIAWLPLQ | |
| | SNQFVRTWDR DMLQQAIERL LSWESWNKRV QEEYAKLKEK MAQLNEQLEG | |
| | GQEWISLLEQ YEENRERELR ENMTAANDKY RITKRQMKGW NELYELWSTF | |
| | PASASHEQYK EALKRVQQRL RGRFGDAHFF QYLMEEKNRL IWKGNPQRIH | |
| | YFVARNELTK RLEEAKQSAT MTLPNARKHP LWVRFDARGG NLQDYYLTAE | |
| | ADKPRSRRFV TFSQLIWPSE SGWMEKKDVE VELALSRQFY QQVKLLKNDK | |
| | GKQKIEFKDK GSGSTFNGHL GGAKLQLERG DLEKEEKNFE DGEIGSVYLN | |
| | VVIDFEPLQE VKNGRVQAPY GQVLQLIRRP NEFPKVTTYK SEQLVEWIKA | |
| | SPQHSAGVES LASGFRVMSI DLGLRAAAAT SIFSVEESSD KNAADFSYWI | |
| | EGTPLVAVHQ RSYMLRLPGE QVEKQVMEKR DERFQLHQRV KFQIRVLAQI | |
| | MRMANKQYGD RWDELDSLKQ AVEQKKSPLD QTDRTFWEGI VCDLTKVLPR | |
| | NEADWEQAVV QIHRKAEEYV GKAVQAWRKR FAADERKGIA GLSMWNIEEL | |
| | EGLRKLLISW SRRTRNPQEV NRFERGHTSH QRLLTHIQNV KEDRLKQLSH | |
| | AIVMTALGYV YDERKQEWCA EYPACQVILF ENLSQYRSNL DRSTKENSTL | |
| | MKWAHRSIPK YVHMQAEPYG IQIGDVRAEY SSRFYAKTGT PGIRCKKVRG | |
| | QDLQGRRFEN LQKRLVNEQF LTEEQVKQLR PGDIVPDDSG ELFMTLTDGS | |
| | GSKEVVFLQA DINAAHNLQK RFWQRYNELF KVSCRVIVRD EEEYLVPKTK | |
| | SVQAKLGKGL FVKKSDTAWK DVYVWDSQAK LKGKTTFTEE SESPEQLEDF | |
| | QEIIEEAEEA KGTYRTLFRD PSGVFFPESV WYPQKDFWGE VKRKLYGKLR | |
| | ERFLTKAR | |
| Alicyclobacillus | MAVKSIKVKL RLDDMPEIRA GLWKLHKEVN AGVRYYTEWL SLLRQENLYR | (SEQ ID |
| acidoterrestris | RSPNGDGEQE CDKTAEECKA ELLERLRARQ VENGHRGPAG SDDELLQLAR | NO: 199) |
| WP_021296342.1 | QLYELLVPQA IGAKGDAQQI ARKFLSPLAD KDAVGGLGIA KAGNKPRWVR | |
| | MREAGEPGWE EEKEKAETRK SADRTADVLR ALADFGLKPL MRVYTDSEMS | |
| | SVEWKPLRKG QAVRTWDRDM FQQAIERMMS WESWNQRVGQ EYAKLVEQKN | |
| | RFEQKNFVGQ EHLVHLVNQL QQDMKEASPG LESKEQTAHY VTGRALRGSD | |
| | KVFEKWGKLA PDAPFDLYDA EIKNVQRRNT RRFGSHDLFA KLAEPEYQAL | |
| | WREDASFLTR YAVYNSILRK LNHAKMFATF TLPDATAHPI WTRFDKLGGN | |
| | LHQYTFLFNE FGERRHAIRF HKLLKVENGV AREVDDVTVP ISMSEQLDNL | |
| | LPRDPNEPIA LYFRDYGAEQ HFTGEFGGAK IQCRRDQLAH MHRRRGARDV | |
| | YLNVSVRVQS QSEARGERRP PYAAVFRLVG DNHRAFVHFD KLSDYLAEHP | |
| | DDGKLGSEGL LSGLRVMSVD LGLRTSASIS VFRVARKDEL KPNSKGRVPF | |
| | FFPIKGNDNL VAVHERSQLL KLPGETESKD LRAIREERQR TLRQLRTQLA | |
| | YLRLLVRCGS EDVGRRERSW AKLIEQPVDA ANHMTPDWRE AFENELQKLK | |
| | SLHGICSDKE WMDAVYESVR RVWRHMGKQV RDWRKDVRSG ERPKIRGYAK | |
| | DVVGGNSIEQ IEYLERQYKF LKSWSFFGKV SGQVIRAEKG SRFAITLREH | |
| | IDHAKEDRLK KLADRIIMEA LGYVYALDER GKGKWVAKYP PCQLILLEEL | |
| | SEYQFNNDRP PSENNQLMQW SHRGVFQELI NQAQVHDLLV GTMYAAFSSR | |
| | FDARTGAPGI RCRRVPARCT QEHNPEPFPW WLNKFVVEHT LDACPLRADD | |
| | LIPTGEGEIF VSPFSAEEGD FHQIHADLNA AQNLQQRLWS DFDISQIRLR | |
| | CDWGEVDGEL VLIPRLTGKR TADSYSNKVF YTNTGVTYYE RERGKKRRKV | |
| | FAQEKLSEEE AELLVEADEA REKSVVLMRD PSGIINRGNW TRQKEFWSMV | |
| | NQRIEGYLVK QIRSRVPLQD SACENTGDI | |
| Alicyclobacillus | MTVRSIRVKL AVGSPQYRDV RRGLWKTHEI MNQGVRYYCE WLVLMRQEPI | (SEQ ID |
| hesperidum | YDEDEHGLTV VQRTREDIQA ELLSRLRTLQ SAHQHSGDMG TDEELLSLMR | NO: 200) |
| WP_074693942.1 | QLYEQLVPSS VDKNKSGDAR MIARNFFNPL TNPNSQGGLG ISNAGRKPKW | |
| | LLKKLSGDPT WEEDYKKAME QKQESSVSFL LLELRRFGLH PIFLPYTDTV | |
| | LEVSWAPKKA RQWVRKWDYD LFQQSIERML SWESWTRRVK ERFEKLVESE | |
| | KKFYDENFAT DPEFIKLAET LEGELQASSQ GFVAVDEHAF QIRPRSMRGF | |
| | DRVADEWCKL ADDAPIEEYE AAIKRVQARL GRNFGSYVLF AHLAKPEYWS | |
| | LWRSDPTKIL RFARLRALQR AVARAKRHAR LTLPDAIHHP IWIRYDAKGK | |
| | NIYSYRLLIP EKRSKRYYVE FSSLIMPDGE NRWAEHRNIR VPLAFSRQWE | |
| | RLHFSIMEDG SLCVQYRDPG VDEPLRAELG GAKIQFDRRY LIRRSSTLSA | |
| | GECGPVYLNV SVDVNPAHRP DVQVLQSAKL VSVSRDTNRI YLRPENLSAY | |
| | WKSQGDGTLP LRVMSVDLGV RSSAAVVICR LEHRDSVVSS GRRTATIYRI | |
| | AGTDEFVAVQ ERAFLLRLPG EGKGTNEDAP LRDVYAQLGT IRQGIQILRS | |
| | LLRLCDTKTP DERQEALHGL AQSLEPSGAW KDELHPHLVM LQGVVHDSVD | |
| | NWKQKVISVH RQMERILGHA VREWKVARKN AGKPPIRRGA GGLSLRRIRQ | |
| | LEQERRTLVA WSNHAREPGQ VVRIKRGTQV AQWLVERVNH LKEDRLKKLA | |
| | DLLIMTALGY VYDETKPSGH KWDKRYPPCQ IILMEDLSRY RFQSDRPPSE | |
| | NSQLMAWSHR RLLEILKLQA DLHKLIVGTV FPAFSSRFDA QSGAPGVRCR | |
| | SVKKQDIENA AQGKGWLARE LQRLNWTLEW LQPNDLIPTG DGELFVTPAC | |
| | CDRQKGIKIV HADLNAAQNL QRRFWGGHAE SLCRVTCDVV ERDGRRYAVP | |
| | RISNAFADSF YKVFGQGVFV STDEEDVYRW MVGEKISSRG RSRGRTSDEE | |
| | AEAETWIDEA REQQGKVIAL FRDASGQIHG GDWLVAKVFW GWVERLVTAR | |
| | LLSRMSEREA AAHKE | |
| Alicyclobacillus | MAVKSMKVKL RLDNMPEIRA GLWKLHTEVN AGVRYYTEWL SLLRQENLYR | (SEQ ID |
| acidiphilus | RSPNGDGEQE CYKTAEECKA ELLERLRARQ VENGHCGPAG SDDELLQLAR | NO: 201) |
| WP_067623834.1 | QLYELLVPQA IGAKGDAQQI ARKFLSPLAD KDAVGGLGIA KAGNKPRWVR | |
| | MREAGEPGWE EEKAKAEARK STDRTADVLR ALADFGLKPL MRVYTDSDMS | |
| | SVQWKPLRKG QAVRTWDRDM FQQAIERMMS WESWNQRVGE AYAKLVEQKS | |
| | RFEQKNFVGQ EHLVQLVNQL QQDMKEASHG LESKEQTAHY LTGRALRGSD | |
| | KVFEKWEKLD PDAPFDLYDT EIKNVQRRNT RRFGSHDLFA KLAEPKYQAL | |
| | WREDASFLTR YAVYNSIVRK LNHAKMFATF TLPDATAHPI WTRFDKLGGN | |
| | LHQYTFLFNE FGEGRHAIRF QKLLTVEDGV AKEVDDVTVP ISMSAQLDDL | |
| | LPRDPHELVA LYFQDYGAEQ HLAGEFGGAK IQYRRDQLNH LHARRGARDV | |
| | YLNLSVRVQS QSEARGERRP PYAAVFRLVG DNHRAFVHFD KLSDYLAEHP | |
| | DDGKLGSEGL LSGLRVMSVD LGLRTSASIS VFRVARKDEL KPNSEGRVPF | |
| | CFPIEGNENL VAVHERSQLL KLPGETESKD LRAIREERQR TLRQLRTQLA | |
| | YLRLLVRCGS EDVGRRERSW AKLIEQPMDA NQMTPDWREA FEDELQKLKS | |
| | LYGICGDREW TEAVYESVRR VWRHMGKQVR DWRKDVRSGE RPKIRGYQKD | |
| | VVGGNSIEQI EYLERQYKFL KSWSFFGKVS GQVIRAEKGS RFAITLREHI | |
| | DHAKEDRLKK LADRIIMEAL GYVYALDDER GKGKWVAKYP PCQLILLEEL | |
| | SEYQFNNDRP PSENNQLMQW SHRGVFQELL NQAQVHDLLV GTMYAAFSSR | |
| | FDARTGAPGI RCRRVPARCA REQNPEPFPW WINKFVAEHK LDGCPLRADD | |
| | LIPTGEGEFF VSPFSAEEGD FHQIHADLNA AQNLQRRLWS DFDISQIRLR | |
| | CDWGEVDGEP VLIPRTTGKR TADSYGNKVF YTKTGVTYYE RERGKKRRKV | |
| | FAQEELSEEE AELLVEADEA REKSVVLMRD PSGIINRGDW TRQKEFWSMV | |
| | NQRIEGYLVK QIRSRVRLQE SACENTGDI | |
| Alicyclobacillus | MAVKSIKVKL MLGHLPEIRE GLWHLHEAVN LGVRYYTEWL ALLRQGNLYR | (SEQ ID |
| macrosporangiidus | RGKDGAQECY MTAEQCRQEL LVRLRDRQKR NGHTGDPGTD EELLGVARRL | NO: 202) |
| SFU30094.1 | YELLVPQSVG KKGQAQMLAS GFLSPLADPK SEGGKGTSKS GRKPAWMGMK | |
| | EAGDSRWVEA KARYEANKAK DPTKQVIASL EMYGLRPLFD VFTETYKTIR | |
| | WMPLGKHQGV RAWDRDMFQQ SLERLMSWES WNERVGAEFA RLVDRRDRFR | |
| | EKHFTGQEHL VALAQRLEQE MKEASPGFES KSSQAHRITK RALRGADGII | |
| | DDWLKLSEGE PVDRFDEILR KRQAQNPRRF GSHDLFLKLA EPVFQPLWRE | |
| | DPSFLSRWAS YNEVLNKLED AKQFATFTLP SPCSNPVWAR FENAEGTNIF | |
| | KYDFLFDHFG KGRHGVRFQR MIVMRDGVPT EVEGIVVPIA PSRQLDALAP | |
| | NDAASPIDVF VGDPAAPGAF RGQFGGAKIQ YRRSALVRKG RREEKAYLCG | |
| | FRLPSQRRTG TPADDAGEVF LNLSLRVESQ SEQAGRRNPP YAAVFHISDQ | |
| | TRRVIVRYGE IERYLAEHPD TGIPGSRGLT SGLRVMSVDL GLRTSAAISV | |
| | FRVAHRDELT PDAHGRQPFF FPIHGMDHLV ALHERSHLIR LPGETESKKV | |
| | RSIREQRLDR LNRLRSQMAS LRLLVRTGVL DEQKRDRNWE RLQSSMERGG | |
| | ERMPSDWWDL FQAQVRYLAQ HRDASGEAWG RMVQAAVRTL WRQLAKQVRD | |
| | WRKEVRRNAD KVKIRGIARD VPGGHSLAQL DYLERQYRFL RSWSAFSVQA | |
| | GQVVRAERDS RFAVALREHI DNGKKDRLKK LADRILMEAL GYVYVTDGRR | |
| | AGQWQAVYPP CQLVLLEELS EYRFSNDRPP SENSQLMVWS HRGVLEELIH | |
| | QAQVHDVLVG TIPAAFSSRF DARTGAPGIR CRRVPSIPLK DAPSIPIWLS | |
| | HYLKQTERDA AALRPGELIP TGDGEFLVTP AGRGASGVRV VHADINAAHN | |
| | LQRRLWENFD LSDIRVRCDR REGKDGTVVL IPRLTNQRVK ERYSGVIFTS | |
| | EDGVSFTVGD AKTRRRSSAS QGEGDDLSDE EQELLAEADD ARERSVVLFR | |
| | DPSGFVNGGR WTAQRAFWGM VHNRIETLLA ERFSVSGAAE KVRG | |
| Sulfobacillus | RQSREDASPQ IIISASDLKA DLLYHARQQQ KEHVPRITGS DAEVLGALRQ | (SEQ ID |
| thermosulfidooxidanS | VYELIVPSSV GKSGDSKTIA RKFLSPLTDP DSAGGRDQSA SGRKPTWTKM | NO: 203) |
| PSR34340.1 | KAEGNPLWEE KFRQWKDRKD NDPTPFVLNQ LADYGLLPLI RLFTDVGENI | |
| | FDPKKPGQFV RPWDRSMFQQ AIERLMSWES WNQRVRQEWE ALTQKHSAFY | |
| | REQFTAEPDA ALYRVAQSLE EEMRKEHQGF ATDAPEAFRI RRVALKGFDR | |
| | LLERWQKTLG KNGQSATLLD DIRRVQSDLG DKFGSAPLYQ KLVDERWQRL | |
| | WTVDPTFLQR YAAFNDLTQR LQRAKRVANL TLPDAVAHPI WSRYEGPNAS | |
| | SGNRYHIHLP TTGQPSSVTF DRILWPDGDG GWYERKRVTV FLRPSHQVDR | |
| | IREAPTDSVV DNFPLVVEDQ SARTILRASW GGAKLEYDRN RLPRQLKKGV | |
| | PDSIYLSLTL NLDTTKPSGL FHMQQNGRVW IRKDVVMQYY NEIPGDNVQF | |
| | KPLYVMSVDL GIRSAAAVSI FSVQLKTGIE EHRLTYPVAD CPGLVAVHER | |
| | SVLLTMPGER REQRDRRYEQ QRQGLRELRT DMRGMNDLLR GAYVDGDRRE | |
| | EFLARLSKLE ETSPELWEPV YRSLNDSKMA PAAEWERLVV YCHRQVEQSL | |
| | SSRIQNLRSG RSAYRMSGGL SLDHVQDLER IRGIIASWTN HPRIPGSVVR | |
| | WQQGRSHTVA LGRHILELKR DRVKKVANYL IMTALGYAYD SKRARGEKWV | |
| | RRYPSCHLMV FEDLTRYRFR TDRPRSENRQ LMRWTHQELI AVTGIQAEPH | |
| | GILVGTMYAG FSSRFDAVTK APGVRGATVR QILRTRGMVR LKEIAADVGV | |
| | DINTLRPHDV LPTGDGEYLL SVVRHRDSYR LKQVHADINA AHNLQRRLWT | |
| | QDEVFRVSCR LALNSERVVA TPPPSYNKRY GKGFFEKGDN GVYIWKTGGK | |
| | IKISDMLEED MDIPEDTAEL LRGNSVTLFR DPSGTIAGGN WLEAKEFWGR | |
| | VNSLVNKGVR DKILGGIPVD NSSAHAE | |
| Spirochaeta sp. | MGLLLPSLSR TVNVTIHLIL HPRKKGSRHR EYAVMLDHAV RKIFLAHNWI | (SEQ ID |
| LUC14_002_19_P3 | KRAEAERQKF EADLYKIDRV PQEARDWLDE FCRERTESTG SIDGYHIRRK | NO: 204) |
| OQX29950.1 | AVLGWEALVE AWDQKDCLSV EDRIAAARDL QDNPGMDKFG DIWLYEALAS | |
| | APCVWQKDGE PNAQILLDYV DAGEAEYKRS HYKVPAYRHP DPLLHPIFCD | |
| | FGQSRWSISF DIHEFKKNGE KNPVNIHALT MGLVSKKRIV KTELKWSSKR | |
| | LNSNLALSLE SPEDAIEVSR ATRLGRAAVG ASQDRAVNIA GLFESAGWNG | |
| | RLQAPRKQLE ALAKLEEDKS AEALAKALRN RIKWFITFSP KLQPHGPWME | |
| | YAERFSGEAP SRAAVIKGKY TVIHQDKTRR RPLAKLHLCR MPGLRVLSVD | |
| | LGHRHAAACA VWETLSSESM EKKCREAGCL PPAPEDLYLH LKKKNKTAVY | |
| | RRIGGNFLPD GNEHPAPWAK LDRQFIIDLQ GEEGCTRMAL AGEIWQVHCM | |
| | EKVFGRSIPL VDRLVRAGWG EKNKQPEILQ ELKQKGWVPL EVSKTNTGYH | |
| | YSLCVDSLMT LAVNTVRFAL RRHACRARIA YYMEGGAIPE GGLPENSGNK | |
| | DFIVEALMLW YELATDSRWN GSWEANFWDE NFDKKLAEIQ DAVNEREGDK | |
| | AKIIKQKERK ELLKKEFIPL AEGLLENSRR ISIASQWRMV WNEEDAIWQS | |
| | ELRSLRDWIL PKGTRGKKRT IRHVGGLSLS RLAVIKSLYR VQKSFYTRMK | |
| | PEGEPMDGTM AVGEGFGQKI LDDLETMKEQ RVKQLASRVV EAALGTGRIK | |
| | KPENNKTPKR PFTAVDEPCH AVVIENLTHY RPENKRTRRE NRQLMTWSSS | |
| | KVKKYLFESC QLHGLYLFEV QASYTSRQDS RTGAPGVRCS ELSVKKFLES | |
| | PFRQREIAHA EENMAQENPC NRYLIALHNK WKNREYDKTA PPLRIPHWGG | |
| | EIFVSALTGN TLQADLNAAA NIGLQALLDP DWPGRWWYVP AVKGCDGRRI | |
| | PHSKCSGAAC LDNWRVGLKN NLYTGVRTPL PGKNKGSTSG EDVHKSNAVE | |
| | KSTINLWRDI SVLPLTEGQW | |
| Bacillus hisashii | MATRSFILKI EPNEEVKKGL WKTHEVLNHG IAYYMNILKL IRQEAIYEHH | (SEQ ID |
| strain C4 v4 | EQDPKNPKKV SKAEIQAELW DFVLKMQKCN SFTHEVDKDE VFNILRELYE | NO: 205) |
| mutant of | ELVPSSVEKK GEANQLSNKF LYPLVDPNSQ SGKGTASSGR KPRWYNLKIA | |
| WP_095142515.1 | GDPSWEEEKK KWEEDKKKDP LAKILGKLAE YGLIPLFIPY TDSNEPIVKE | |
| K846R | IKWMEKSRNQ SVRRLDKDMF IQALERFLSW ESWNLKVKEE YEKVEKEYKT | |
| S893R | LEERIKEDIQ ALKALEQYEK ERQEQLLRDT LNTNEYRLSK RGLRGWREII | |
| E837G | QKWLKMDENE PSEKYLEVFK DYQRKHPREA GDYSVYEFLS KKENHFIWRN | |
| | HPEYPYLYAT FCEIDKKKKD AKQQATFTLA DPINHPLWVR FEERSGSNLN | |
| | KYRILTEQLH TEKLKKKLTV QLDRLIYPTE SGGWEEKGKV DIVLLPSRQF | |
| | YNQIFLDIEE KGKHAFTYKD ESIKFPLKGT LGGARVQFDR DHLRRYPHKV | |
| | ESGNVGRIYF NMTVNIEPTE SPVSKSLKIH RDDFPKVVNF KPKELTEWIK | |
| | DSKGKKLKSG IESLEIGLRV MSIDLGQRQA AAASIFEVVD QKPDIEGKLF | |
| | FPIKGTELYA VHRASFNIKL PGETLVKSRE VLRKAREDNL KLMNQKLNFL | |
| | RNVLHFQQFE DITEREKRVT KWISRQENSD VPLVYQDELI QIRELMYKPY | |
| | KDWVAFLKQL HKRLEVEIGK EVKHWRKSLS DGRKGLYGIS LKNIDEIDRT | |
| | RKFLLRWSLR PTEPGEVRRL EPGQRFAIDQ LNHLNALKED RLKKMANTII | |
| | MHALGYCYDV RKKKWQAKNP ACQIILFEDL SNYNPYGERS RFENSRLMKW | |
| | SRREIPRQVA LQGEIYGLQV GEVGAQFSSR FHAKTGSPGI RCRVVTKEKL | |
| | QDNRFFKNLQ REGRLTLDKI AVLKEGDLYP DKGGEKFISL SKDRKCVTTH | |
| | ADINAAQNLQ KRFWTRTHGF YKVYCKAYQV DGQTVYIPES KDQKQKIIEE | |
| | FGEGYFILKD GVYEWVNAGK LKIKKGSSKQ SSSELVDSDI LKDSFDLASE | |
| | LKGEKLMLYR DPSGNVFPSD KWMAAGVFFG KLERILISKL TNQYSISTIE | |
| | DDSSKQSM | |

TABLE 7
Cas12c (C2c3) orthologs

| | | |
|---|---|---|
| OspCas12c | MTKLRHRQKK LTHDWAGSKK REVLGSNGKL QNPLLMPVKK GQVTEFRKAF | (SEQ ID |
| AWU30132.1 | SAYARATKGE MTDGRKNMFT HSFEPFKTKP SLHQCELADK AYQSLHSYLP | NO: 206) |
| KZX85786.1 | GSLAHFLLSA HALGFRIFSK SGEATAFQAS SKIEAYESKL ASELACVDLS | |
| | IQNLTISTLF NALTTSVRGK GEETSADPLI ARFYTLLTGK PLSRDTQGPE | |
| | RDLAEVISRK IASSFGTWKE MTANPLQSLQ FFEEELHALD ANVSLSPAFD | |
| | VLIKMNDLQG DLKNRTIVFD PDAPVFEYNA EDPADIIIKL TARYAKEAVI | |
| | KNQNVGNYVK NAITTTNANG LGWLLNKGLS LLPVSTDDEL LEFIGVERSH | |
| | PSCHALIELI AQLEAPELFE KNVFSDTRSE VQGMIDSAVS NHIARLSSSR | |
| | NSLSMDSEEL ERLIKSFQIH TPHCSLFIGA QSLSQQLESL PEALQSGVNS | |
| | ADILLGSTQY MLTNSLVEES IATYQRTLNR INYLSGVAGQ INGAIKRKAI | |
| | DGEKIHLPAA WSELISLPFI GQPVIDVESD LAHLKNQYQT LSNEFDTLIS | |
| | ALQKNFDLNF NKALLNRTQH FEAMCRSTKK NALSKPEIVS YRDLLARLTS | |
| | CLYRGSLVLR RAGIEVLKKH KIFESNSELR EHVHERKHFV FVSPLDRKAK | |
| | KLLRLTDSRP DLLHVIDEIL QHDNLENKDR ESLWLVRSGY LLAGLPDQLS | |
| | SSFINLPIIT QKGDRRLIDL IQYDQINRDA FVMLVTSAFK SNLSGLQYRA | |
| | NKQSFVVTRT LSPYLGSKLV YVPKDKDWLV PSQMFEGRFA DILQSDYMVW | |
| | KDAGRLCVID TAKHLSNIKK SVFSSEEVLA FLRELPHRTF IQTEVRGLGV | |
| | NVDGIAFNNG DIPSLKTFSN CVQVKVSRTN TSLVQTLNRW FEGGKVSPPS | |
| | IQFERAYYKK DDQIHEDAAK RKIRFQMPAT ELVHASDDAG WTPSYLLGID | |
| | PGEYGMGLSL VSINNGEVLD SGFIHINSLI NFASKKSNHQ TKVVPRQQYK | |
| | SPYANYLEQS KDSAAGDIAH ILDRLIYKLN ALPVFEALSG NSQSAADQVW | |
| | TKVLSFYTWG DNDAQNSIRK QHWFGASHWD IKGMLRQPPT EKKPKPYIAF | |
| | PGSQVSSYGN SQRCSCCGRN PIEQLREMAK DTSIKELKIR NSEIQLFDGT | |
| | IKLFNPDPST VIERRRHNLG PSRIPVADRT FKNISPSSLE FKELITIVSR | |
| | SIRHSPEFIA KKRGIGSEYF CAYSDCNSSL NSEANAAANV AQKFQKQLFF | |
| | EL | |
| QFN42172.1 | MRSNYHGGRN ARQWRKQISG LARRTKETVF TYKFPLETDA AEIDFDKAVQ | (SEQ ID |
| | TYGIAEGVGH GSLIGLVCAF HLSGFRLFSK AGEAMAFRNR SRYPTDAFAE | NO: 207) |
| | KLSAIMGIQL PTLSPEGLDL IFQSPPRSRD GIAPVWSENE VRNRLYTNWT | |
| | GRGPANKPDE HLLEIAGEIA KQVFPKFGGW DDLASDPDKA LAAADKYFQS | |
| | QGDFPSIASL PAAIMLSPAN STVDFEGDYI AIDPAAETLL HQAVSRCAAR | |
| | LGRERPDLDQ NKGPFVSSLQ DALVSSQNNG LSWLFGVGFQ HWKEKSPKEL | |
| | IDEYKVPADQ HGAVTQVKSF VDAIPLNPLF DTTHYGEFRA SVAGKVRSWV | |
| | ANYWKRLLDL KSLLATTEFT LPESISDPKA VSLFSGLLVD PQGLKKVADS | |
| | LPARLVSAEE AIDRLMGVGI PTAADIAQVE RVADEIGAFI GQVQQFNNQV | |
| | KQKLENLQDA DDEEFLKGLK IELPSGDKEP PAINRISGGA PDAAAEISEL | |
| | EEKLQRLLDA RSEHFQTISE WAEENAVTLD PIAAMVELER LRLAERGATG | |
| | DPEEYALRLL LQRIGRLANR VSPVSAGSIR ELLKPVFMEE REFNLFFHNR | |
| | LGSLYRSPYS TSRHQPFSID VGKAKAIDWI AGLDQISSDI EKALSGAGEA | |
| | LGDQLRDWIN LAGFAISQRL RGLPDTVPNA LAQVRCPDDV RIPPLLAMLL | |
| | EEDDIARDVC LKAFNLYVSA INGCLFGALR EGFIVRTRFQ RIGTDQIHYV | |
| | PKDKAWEYPD RLNTAKGPIN AAVSSDWIEK DGAVIKPVET VRNLSSTGFA | |
| | GAGVSEYLVQ APHDWYTPLD LRDVAHLVTG LPVEKNITKL KRLTNRTAFR | |
| | MVGASSFKTH LDSVLLSDKI KLGDFTIIID QHYRQSVTYG GKVKISYEPE | |
| | RLQVEAAVPV VDTRDRTVPE PDTLFDHIVA IDLGERSVGF AVFDIKSCLR | |
| | TGEVKPIHDN NGNPVVGTVA VPSIRRLMKA VRSHRRRRQP NQKVNQTYST | |
| | ALQNYRENVI GDVCNRIDTL MERYNAFPVL EFQIKNFQAG AKQLEIVYGS | |
| QFN42158.1 | MKKFELKQNF RNNYSGKTLR NFRQTLAQIA NKKSSDSILT IKFKLDCSKT | (SEQ ID |
| | GKLPKYENLI SLYDTIEDIK KGTLSYYLFT LIVSGFKFFG SASQAKAFST | NO: 208) |
| | KDIFKDNDFY NQFKIQSHLD LPDFVPSKIY QRLKKNVRST NGKDNAFKAS | |
| | VIVAEYRKEI GKLKNKDESS EHQCEELFKK IGTALETRFS SWQDLINNCS | |
| | TGCEIIDEIL NDSFGTLPSI KKMVLASTTQ SSDGEQDGIA IAYDPDSTFI | |
| | KSDELLNPYF AVATILKSMP PEIQQDKKSA YVKANLTTPT HNALSWIFGK | |
| | GLTLFQTEST EKLCAMFNVS DKRVIEQVQD AAKAVKLPAE LDLNHCTLKF | |
| | QDFRSSLGGH LDSWTTNYLK RLDELNDLLL NLPKNLSLPD IFMIDGKDFI | |
| | EYSGCNRDEI QQMIDFVVNE QNRIKLQESL NALLGKGNNQ ICSDDISTVK | |
| | DFSEIVNSLH SFVQQIDNSL EQSSNEANSI FSELKKKIEK NEKWDIWKNN | |
| | LKKIPKLNKL SGGVPDAWKE IREIEQKFHE ISENQKKHFT EVMEWIDAGN | |
| | GTIDIFESRF KYDELLKKSK KNNLQSADEL AFRSVLNKLG RFARQGNDLV | |
| | CEKIKNWFKE QNIFDSSKDF NRYFINQKGF IFKHPSSKKD NSPYNLSANL | |
| | LEKRYEVTNT VGALLEQCES DPAIVNDPFS MRSLVEFRAL WFSINISGIS | |
| | KEQHIPTKIA QPKLDDSTYQ ESVSPTLKYR LEKEQITSSE LNSIFTVYKS | |
| | LLSGLSIRLS RNSFYLRTKF SWIGNNSLIY CPKETTWKIP AAYFKSDLWN | |
| | EYKDKQILIV NEEYDVDVVK TFESVYKIVK SKDNNEKNRI LPLLKQLPHD | |
| | WMFKLPFGAS NAEKCKVLKL EKNNKKFKPL SVSKDSLARL SGPSTYFNQI | |
| | DEIMMNDESE LSEMTLLADE PVRQQMSNGK IEIIPDDYVM SLAIPITRSL | |
| | KKGNTESFPF KNIVSIDQGE AGFAYAVFKL SDCGNERAEP IATGLIPIPS | |
| | IRRLIHSVKK YRGKKQRIQN FNQKFDSTMF TLRENVTGDI CGLIVALMKK | |
| | YNAFPILEKQ VGNLESGSKQ LMLVYKAVNS KFLAAKVDMQ NDQRRSWWYQ | |
| | GNSWNTPILR ISNPNQSNNK NIVKNINGKK YEELKIYPGY SVSAYMTSCI | |
| | CHVCGRNALE LLKNDDSTGK VKKYQINQDG EVTIGGEVIK LYRKPDRLTP | |
| | VKNLAKKGNR ERTYASINER APMSKDTTQS RYFCVFKNCP CHNKEQHADV | |
| | NAAINIGRRF LKDCILDDNK EKD | |
| QFN42173.1 | MNARDWRKHV GVLAQQHKET TRTYTFPLDT TGSAIDFDAA LQAYNAVEGV | (SEQ ID |
| | GYGSLLGLAC AVHLSGFRLF STGKEAATFR NRARYPNAAF QAALRKELGT | NO: 209) |
| | TITTLTPETL DRLFSSRPKR RNGVPLPWNQ DSIRDRLYTN WVKPRPGDTP | |
| | DAVLFQIATG IAQEITEDVS SWTDLAKNSD RGLKAAHRYF ARVGGFPAFD | |
| | NLTPPATVQP TDTTIDYDPN APFHLVSHAD QTLIHQSISL CAHRIRQEDP | |
| | ALDPNKSGFI KQLQNNFLSQ TFYGLSWLFG AGYVHFRECT ANDLAIQYGI | |
| | PNNCRDGIHQ IKSFADAILP NTFFEKKHYR KDSRSVGKKA KSWISNYWQR | |
| | LLQLQTWVDD HTWVTLPQEL TEAQFKPLFR GLLVDAVELM AIAERLPQRL | |
| | ADCRDSLDCL MGKGPQAATK NDVEIVEKVR EEIESFVGQI EQLGNQLRHQ | |
| | LENENNDQVH RDNLHQLKNR LPLDLRRPQA LNKISGGVPD VAKSIRGLET | |
| | QLDQVLKERR SHFGRLTKWA KECGITLDPL QPLIESEKQR VAERGSAHDA | |
| | KELAIRLLLQ RIGRLGHRLS PTNATAIQEL LRPVFAVKRE FNLFFHNHMG | |
| | ALYRSPYSTS RHQPFQINVD VAHGTDWIGT IETLIQNLFT QIQDDALLRD | |
| | LVQLEGFVFS HKLRALPGVI PSELARPNNL QQMGLPALLL VLLQADQVHR | |
| | ETVLRVFNLY GSAINGYLFQ ALRPGFIVRA GFQRLETKKL RYVPKAQSWQ | |
| | YPDRLHHAKS AIKNSLSAGW IKKNHQGAIL PQKTLTALVK QKSLKDTGVP | |
| | EYLVQAPHDW YVPIDLRGPA IPIEGLTVGT EGPELTQLGP MKDDCAFRAI | |
| | GPSSFKSKID AGLLPQDVKY GDMTLIFDQH YQQSISFANG TFSIQYQPTS | |
| | LQVKAAIPVV DKRPRDTRNN SHLYDRIVAI DLGERKIGYA IFDLKQVLKS | |
| | EQLEPMREDG KPLIGSISIR SIRGLMKAVQ THRNRRQPNY RIDQTYSKAL | |
| | MHYRESVIGD VCNAIDTLCA RYGGFPVLES SVRNFEVGSA QLKTVYGSVS | |
| | RRYTWSAVDA HKNQRQQYWL GGTKDKIPIW THPYLMTREW DEKNSKWSNR | |
| | SKPLKMHPGV EVHPAGTSQI CHQCKRNPIG ALWNVADTVV LDDQGQLDLD | |
| | DGTIRLNSGY IDTTEIKRAR RKKIRLPENK PLTGSHKTSH VRAVARRNLR | |
| | QPPKSTRAKD TTQSRYTCLY VDCGHECHAD ENAAINIGRK YLQERIHIEA | |
| | SRQALSTR | |
| QFN42174.1 | MVAGLKKIKR DGVTMKSNYH GGVKARAWRK RIGGLARRQK ETVFTYKFPL | (SEQ ID |
| | ETEEAGIDFD KAVQTYGIAE GISQGSLIGL VCAFHLSGFR LFSKADETKA | NO: 210) |
| | FCNQGRYPNQ AFAEKLRNEL SVTLPKLSPQ SLDVLFQSSP KSKNGVAPEW | |
| | SKNAIRNRLY TNWTGKGAGT NPDEHLLEIA EDIAAEIDSD LDGWKDLEEH | |
| | PEKGLSAADR YFQAQGDFPS LTGLPPSVPL TPQNSTVAFE GDPVCLNPSD | |
| | NTLLHQAVAR CAGRILQEQP NLSPDKNRFI NQLQDELVSS QNNGLSWLFG | |
| | VGFKYWKEMS VDQLADDYKV KSTDLDALKQ VKSFIDAIPL NPLFDTPHYG | |
| | EFRASVAGKM RSWVKNYWKR LLDLKSQLGT ANINLPEGLD EQRAENLFSG | |
| | LLIDSKGLRQ VTDKLPSRLK KAEDTIDRLM GDGNPTSDDI EQVETVAAEI | |
| | SAFIGQVEQF NNQLEQRLEN PLEGDDETFL KQLKIDLPAE FKKPPAINRI | |
| | SGGSPDPTAE IAELEEKLDR LMSARKEHYE TIAEWASANK VTLDPMEAMT | |
| | TLEAQRLTER GAEGDQEEFA LRLLLQRIGR LANRLSPQGA TAIRDLLRPV | |
| | FTEKREFNLF FHNRMGSLYR SPYSTSRHQP FTIDVAVAKN TDWMDALDGI | |
| | AETIMKGLSQ AGDELSLRQL EEDEVSREVC LKAFNLYVSA INGCLFRALR | |
| | EGFIVRTKFQ RLERDVLSYV PKTKLWNYPQ RLDTARGPIH SALAAAWINK | |
| | EGSVIDPVET VTALSDTGFS DDGIPEYLVQ APHDWYLRDW INISGFSLSQ | |
| | RLRGLPDTVP GELALVRSAD DVRIPPMLAL TPIDLRDISK PVSGLPVKKN | |
| | ITGLKRQKKQ TAFRMVGPSS FKSHLDSTLL SEEVKLGDFT LIFDQYYKQR | |
| | VSYNGRVKIT FEPDRLHVEA AVPVIDKRVR PSTEEDALFD HLLAIDLGEK | |
| | RVGYAVYDIK ACLRTGDIKP LEDGDGKPIV GSVAVPSIRR LMKAVRSHRQ | |
| | QRQPNQKVNQ TYSTALMNYR ENVIGDVCNR IDTLMEKYNA FPVLESSVMN | |
| | FEAGSRQLEM VYGSVLHRYT YSKIDAHTAK RKEYWYTGEY WDHPYLMAHK | |
| | WNERTRSYSG SLSALTLYPG VMVHPAGTSQ RCHQCKRNPM VEIKQLTGQV | |
| | EINADGSLEL DDGTICLYEG YDYSPEEYKK AKREKRRLDP NVPLSGRHQA | |
| | KHVSAVAKRN LRRPTVSMMS GDTTQARYVC LYTDCDFTGH ADENAAINIG | |
| | WKYLTERIAL SESKDKAGV | |

TABLE 8
Cas12e (CasY) orthologs

| | | |
|---|---|---|
| APG80656.1 | MSKRHPRISG VKGYRLHAQR LEYTGKSGAM RTIKYPLYSS PSGGRTVPRE | (SEQ ID |
| GI: 1110962136 | IVSAINDDYV GLYGLSNFDD LYNAEKRNEE KVYSVLDFWY DCVQYGAVFS | NO: 211) |
| QFN42175.1 | YTAPGLLKNV AEVRGGSYEL TKTLKGSHLY DELQIDKVIK FLNKKEISRA | |
| | NGSLDKLKKD IIDCFKAEYR ERHKDQCNKL ADDIKNAKKD AGASLGERQK | |
| | KLFRDFFGIS EQSENDKPSF TNPLNLTCCL LPFDTVNNNR NRGEVLFNKL | |
| | KEYAQKLDKN EGSLEMWEYI GIGNSGTAFS NFLGEGFLGR LRENKITELK | |
| | KAMMDITDAW RGQEQEEELE KRLRILAALT IKLREPKFDN HWGGYRSDIN | |
| | GKLSSWLQNY INQTVKIKED LKGHKKDLKK AKEMINRFGE SDTKEEAVVS | |
| | SLLESIEKIV PDDSADDEKP DIPAIAIYRR FLSDGRLTLN RFVQREDVQE | |
| | ALIKERLEAE KKKKPKKRKK KSDAEDEKET IDFKELFPHL AKPLKLVPNF | |
| | YGDSKRELYK KYKNAAIYTD ALWKAVEKIY KSAFSSSLKN SFFDTDFDKD | |
| | FFIKRLQKIF SVYRRFNTDK WKPIVKNSFA PYCDIVSLAE NEVLYKPKQS | |
| | RSRKSAAIDK NRVRLPSTEN IAKAGIALAR ELSVAGFDWK DLLKKEEHEE | |
| | YIDLIELHKT ALALLLAVTE TQLDISALDF VENGTVKDFM KTRDGNLVLE | |
| | GRFLEMFSQS IVFSELRGLA GLMSRKEFIT RSAIQTMNGK QAELLYIPHE | |
| | FQSAKITTPK EMSRAFLDLA PAEFATSLEP ESLSEKSLLK LKQMRYYPHY | |
| | FGYELTRTGQ GIDGGVAENA LRLEKSPVKK REIKCKQYKT LGRGQNKIVL | |
| | YVRSSYYQTQ FLEWFLHRPK NVQTDVAVSG SFLIDEKKVK TRWNYDALTV | |
| | ALEPVSGSER VFVSQPFTIF PEKSAEEEGQ RYLGIDIGEY GIAYTALEIT | |
| | GDSAKILDQN FISDPQLKTL REEVKGLKLD QRRGTFAMPS TKIARIRESL | |
| | VHSLRNRIHH LALKHKAKIV YELEVSRFEE GKQKIKKVYA TLKKADVYSE | |
| | IDADKNLQTT VWGKLAVASE ISASYTSQFC GACKKLWRAE MQVDETITTQ | |
| | ELIGTVRVIK GGTLIDAIKD FMRPPIFDEN DTPFPKYRDF CDKHHISKKM | |
| | IKVLGQMKKI FCRANADADI QASQTIALLR YVKEEKKVED YFERFRKLKN | |
| | RGNSCLFICP | |

### 6.4. Protospacer Adjacent Motif

As used herein, the term “protospacer adjacent sequence” or “protospacer adjacent motif” or “PAM” refers to an approximately 2-6 base pair DNA sequence (or a 2-, 3—, 4-, 5-, 6-, 7-, 8-, 9-, 10-, 11-, 12-long nucleotide sequence) that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease may be modified to alter the PAM specificity of the nuclease such that the nuclease recognizes alternative PAM sequence.

For example, with reference to the canonical SpCas9 amino acid sequence, the PAM specificity can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities and in some embodiments are therefore chosen based on the desired PAM recognition. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These examples are not meant to be limiting. It will be further appreciated that non-SpCas9s bind a variety of PAM sequences, which makes them useful to expand the range of sequences that can be targeted according to the invention. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10 (5): 891-899 (which is incorporated herein by reference). Gasiunas used cell-free biochemical screens to identify protospacer adjacent motif (PAM) and guide RNA requirements of 79 Cas9 proteins. (Gasiunas et al., A catalogue of biochemically diverse CRISPR-Cas9 orthologs, Nature Communications 11:5512 doi.org/10.1038/s41467-020-19344-1) The authors described 7 classes of gRNA and 50 different PAM requirement.

Oh, Y. et al. describe linking reverse transcriptase to a Francisella novicida Cas9 [FnCas9 (H969A)] nickase module. (Oh, Y. et al., Expansion of the prime editing modality with Cas9 from Francisella novicida, bioRxiv 2021.05.25.445577; doi.org/10.1101/2021.05.25.445577). By increasing the distance to the PAM, the FnCas9 (H969A) nickase module expands the region of a reverse transcription template (RTT) following the primer binding site.

### 6.5. Prime Editors

“Prime editor fusion protein” describes a protein that is used in prime editing. Prime editing uses CRISPR enzyme that nicks or cuts only single strand of double stranded DNA, i.e., a nickase; and a nickase can occur either naturally or by mutation or modification of a nuclease that makes double stranded cuts. Such an enzyme can be a catalytically-impaired Cas9 endonuclease (a nickase). Such an enzyme can be a Casl2a/b, MAD7, or variant thereof. The nickase is fused to an engineered reverse transcriptase (RT). The nickase is programmed (directed) with a prime-editing guide RNA (pegRNA). The skilled person in the art would appreciate that the pegRNA both specifies the target site and encodes the desired edit. Advantageously the nickase is a catalytically-impaired Cas9 endonuclease, a Cas9 nickase, that is fused to the reverse transcriptase. During genetic editing, the Cas9 nickase part of the protein is guided to the DNA target site by the pegRNA, whereby a nick or single stranded cut occurs. The reverse transcriptase domain then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, optionally, the prime editor (PE) guides resolution of the heteroduplex to favor copying the edit onto the unedited strand, completing the process (typically achieved with a nickase gRNA).

As used herein, “PE1” refers to a PE complex comprising a fusion protein comprising Cas9 (H840A) and a wild type MMLV_RT having the following N-terminus to C-terminus structure: [NLS]-[Cas9 (H840A)]-[linker]-[MMLV_RT (wt)]+a desired PEgRNA. In various embodiments, the prime editors disclosed herein is comprised of PE1.

As used herein, “PE2” refers to a PE complex comprising a fusion protein comprising Cas9 (H840A) and a variant MMLV_RT having the following N-terminus to C-terminus structure: [NLS]-[Cas9 (H840A)]-[linker]-[MMLV_RT (D200N) (T330P) (L603W) (T306K) (W313F)]+a desired PEgRNA. In various embodiments, the prime editors disclosed herein is comprised of PE2.

In various embodiments, the prime editors disclosed herein is comprised of PE2 and co-expression of MMR protein MLH1dn, that is PE4.

As used herein, “PE3” refers to PE2 plus a second-strand nicking guide RNA that complexes with the PE2 and introduces a nick in the non-edited DNA strand. The induction of the second nick increases the chances of the unedited strand, rather than the edited strand, to be repaired. In various embodiments, the prime editors disclosed herein is comprised of PE3.

In various embodiments, the prime editors disclosed herein is comprised of PE3 and co-expression of MMR protein MLH1dn, that is PE5.

As used herein, “PE3b” refers to PE3 but wherein the second-strand nicking guide RNA is designed for temporal control such that the second strand nick is not introduced until after the installation of the desired edit. This is achieved by designing a gRNA with a spacer sequence with mismatches to the unedited original allele that matches only the edited strand. Using this strategy, mismatches between the protospacer and the unedited allele should disfavor nicking by the sgRNA until after the editing event on the PAM strand takes place.

### 6.6. Guides for Prime Editing

Anzalone et al., 2019 (Nature 576:149) describes prime editing and a prime editing complex using a type II CRISPR and can be used herein. A prime editing complex consists of a type II CRISPR PE protein containing an RNA-guided DNA-nicking domain fused to a reverse transcriptase (RT) domain and complexed with a pegRNA. The pegRNA comprises (5′ to 3′) a spacer that is complementary to the target sequence of a genomic DNA, a nickase (e.g. Cas9) binding site, a reverse transcriptase template including editing positions, and primer binding site (PBS). The PE-pegRNA complex binds the target DNA and the CRISPR protein nicks the PAM-containing strand. The resulting 3′ end of the nicked target hybridizes to the primer-binding site (PBS) of the pegRNA, then primes reverse transcription of new DNA containing the desired edit using the RT template of the pegRNA. The overall structure of the pegRNA is like that of a typical type II sgRNA with a reverse transcriptase template/primer binding site appended to the 3′ end. The structure leaves the PBS at the 3′ end of the pegRNA free to bind to the nicked strand complementary to the target which forms the primer for reverse transcription.

Guide RNAs of CRISPRs differ in overall structure. For example, while the spacer of a type II gRNA is located at the 5′ end, the spacer of a type V gRNA is located towards the 3′ end, with the CRISPR protein (e.g. Cas12a) binding region located toward the 5′ end. Accordingly, the regions of a type V pegRNA are rearranged compared to a type II pegRNA. The overall structure of the pegRNA is like that of a typical type II sgRNA with a reverse transcriptase template/primer binding site appended to the 3′ end. The pegRNA comprises (5′ to 3′) a CRISPR protein-binding region, a spacer which is complementary to the target sequence of a genomic DNA, a reverse transcriptase template including editing positions, and primer binding site (PBS).

In typical embodiments, the guide RNA (e.g., atgRNA) or guide RNA complex is capable of binding a DNA binding nickase selected from the group consisting of: Cas9-D10A, Cas9-H840A, Cas12a/b/c/d/e nickase, CasX nickase, SaCas9 nickase, and CasY nickase. In certain embodiments, the nickase is linked or fused to one or more of a reverse transcriptase. In certain embodiments, the nickase is linked or fused to one or more of a reverse transcriptase and integrase. In certain embodiments, the nickase is linked or fused to one or more of an integrase.

6.7. Attachment Site-Containing Guide RNA (atgRNA)

As used herein, the term “attachment site-containing guide RNA” (atgRNA) and the like refer to an extended single guide RNA (sgRNA) comprising a primer binding site (PBS), a reverse transcriptase (RT) template sequence, and wherein the RT template encodes for an integration recognition site or a recombinase recognition site that can be recognized by a recombinase, integrase, or transposase. In some embodiments, the RT template comprises a clamp sequence and an integration recognition site. As referred to herein an atgRNA may be referred to as a guide RNA. An integration recognition site or recombinase target recognition site incorporated into the pegRNA is referred to as an attachment site containing guide RNA (atgRNA).

As used herein, the term “cognate integrase recognition site” or “integration cognate” or “cognate pair” refers to a first integrase recognition site (e.g., any of the integrase recognition sites described herein) and a second integrase recognition site (e.g., any of the integrase recognition sites described herein) that can be recombined. Recombination between a first integrase recognition site (e.g., any of the integrase recognition sites described herein) and a second recognition site (e.g., any of the integrase recognition sites described herein) is mediated by functional symmetry between the two integrase recognition sites and the central dinucleotide of each of the two integrase recognition sites. In some cases, a first integrase recognition site (e.g., any of the integrase recognition sites described herein) that can be recombined with a second integrase recognition site (e.g., any of the integrase recognition sites described herein) are referred to as a “cognate pair.” A non-limiting example of a cognate pair include an attB site and an attP site, whereby a B×B1 integrase mediates recombination between the attB site and the attP site.

In some cases, a single nucleic acid construct includes a first cognate pair (e.g., a first integrase recognition site and a second integrase recognition site) and a second cognate pair (e.g., a third integrase recognition site and a fourth recognition site). In such cases, the first cognate pair and the second cognate pair have different central dinucleotides that enable recombination only with the other integrase recognition site within the cognate pair.

In typical embodiments, an atgRNA comprises a reverse transcriptase template that encodes, partially or in its entirety, an integration recognition site (also referred to as an integration target recognition site) or a recombinase recognition site (also referred to as a recombinase target recognition site). The integration target recognition site, which is to be place at a desired location in the genome, is referred to as a “beacon” site or an “attachment site” or a “landing pad” or “landing site.” An integration target recognition site or recombinase target recognition site incorporated into the pegRNA is referred to as an attachment site containing guide RNA (atgRNA).

During genome editing, the primer binding site allows the 3′ end of the nicked DNA strand to hybridize to the atgRNA, while the RT template serves as a template for the synthesis of edited genetic information. The atgRNA is capable for instance, without limitation, of (i) identifying the target nucleotide sequence to be edited and (ii) encoding new genetic information that replaces (or in some cases adds) the targeted sequence. In some embodiments, the atgRNA is capable of (i) identifying the target nucleotide sequence to be edited and (ii) encoding an integration site that replaces (or inserts/deletes within) the targeted sequences.

In some embodiments, the single nucleic acid construct (i.e., “installer”) contains a nucleotide sequence encoding an attachment site-containing guide RNA (atgRNA). In some embodiments, the atgRNA comprises a domain that is capable of guiding the prime editor fusion protein to a target sequence, thereby identifying the target nucleotide sequence to be edited; and a reverse transcriptase (RT) template that comprises a first integration recognition site. In some embodiments, the atgRNA comprises a domain that is capable of guiding the prime editor fusion protein to a target sequence, thereby identifying the target nucleotide sequence to be edited; and a reverse transcriptase (RT) template that comprises at least a portion first integration recognition site.

In some embodiments, the single nucleic acid construct (i.e., “installer”) contains a contains a nucleotide sequence encoding a first attachment site-containing guide RNA (atgRNA) and a nucleotide sequence encoding a second attachment site-containing guide RNA (atgRNA). In some embodiments, where the single nucleic acid construct (i.e., “installer”) contains a first atgRNA and a second atgRNA, the first atgRNA and the second atgRNA are an at least first pair of atgRNAs, where the at least first pair of atgRNAs have domains that are capable of guiding the gene editor protein or prime editor fusion protein to a target sequence, the first atgRNA further includes a first RT template that comprises at least a portion of the first integration recognition site; and the second atgRNA further includes a second RT template that comprises at least a portion of the first integration recognition site, and the first atgRNA and the second atgRNAs collectively encode the entirety of the first integration recognition site.

In some embodiments, the first atgRNA's reverse transcriptase template encodes for a first single-stranded DNA sequence (i.e., a first DNA flap) that contains a complementary region to a second single-stranded DNA sequence (i.e., a second DNA flap) encoded by a second atgRNA comprising a second reverse transcriptase template. In certain embodiments, the complementary region between the first and second single-stranded DNA sequences is comprised of more than 5 consecutive bases of an integrase target recognition site. In certain embodiments, the complementary region between the first and second single-stranded DNA sequences is comprised of more than 10 consecutive bases of an integrase target recognition site. In certain embodiments, the complementary region between the first and second single-stranded DNA sequences is comprised of more than 20 consecutive bases of an integrase target recognition site. In certain embodiments, the complementary region between the first and second single-stranded DNA sequences is comprised of more than 30 consecutive bases of an integrase target recognition site. Use of two guide RNAs that are (or encode DNA that is) partially complementarity to each other and comprised of consecutive bases of an integrase target recognition site are referred to as dual, paired, annealing, complementary, or twin attachment site-containing guide RNAs (atgRNAs). In certain embodiments, use of two guide RNAs that are (or encode DNA that is) full complementarity to each other and comprised of consecutive bases of an integrase target recognition site are referred to as dual, paired, annealing, complementary, or twin attachment site-containing guide RNAs (atgRNAs).

In some embodiments, upon introducing the nucleic acid construct into a cell, the first atgRNA incorporates the first integrase recognition site into the cell's genome at the target sequence.

In some embodiments, upon introducing the nucleic acid construct into a cell, the first pair of atgRNAs incorporate the first integrase recognition site into the cell's genome at the target sequence.

Table 9 includes atgRNAs, sgRNAs and nicking guides that can be used herein. Spacers are labeled in capital font (SPACER), RT regions in bold capital (RT REGION), AttB sites in bold lower case (attB site), and PBS in capital italics (PBS). Unless otherwise denoted, the AttB is for Bxb1.

TABLE 9

| | | SEQ |
|---|---|---|
| Description | Sequence (5′-3′) | ID NO: |
| ACTB N- | GCTATTCTCGCAGCTCACCAgttttagagctagaaatagcaagttaaaataaggctagtc | 212 |
| term PBS | cgttatc | |
| 13 RT | aacttgaaaaagtggcaccgagtcggtgcGACGAGCGCGGCGATATCATCATC | |
| 29 AttB 46 | CATGGccggatgatcctgacgacggagaccgccgtcgtcgacaagccggccTGAGCTGC | |
| atgRNA | GAGAA | |
| ACTB N- | GCTATTCTCGCAGCTCACCAgtttgagagctatgctggaaacagcatagcaagttcaaat | 213 |
| term PBS | aaggc | |
| 13 RT | tagtccgttatcaacttgaaaaagtggcaccgagtcggtgcGACGAGCGCGGCGATAT | |
| 29 AttB 46 | CATCATCCATGGccggatgatcctgacgacggagaccgccgtcgtcgacaagccggcc | |
| atgRNA | TGAGCTGCGA GAA | |
| with v2 | | |
| scaffold | | |
| ACTB N- | GCTATTCTCGCAGCTCACCAgttttagagctagaaatagcaagttaaaataaggctagtc | 214 |
| term | cgttatc | |
| PBS_13_RT_ | aacttgaaaaagtggcaccgagtcggtgcGAGTCGGTGCGACGAGCGCGGC | |
| 29_with | GATATCATCATCCATGGcacaattaacatctcaatcaaggtaaaTGCTTGAGC | |
| TP901-1 | TGCGAGAA | |
| minimal | | |
| AttB f | | |
| atgRNA | | |
| ACTB N- | GCTATTCTCGCAGCTCACCAgttttagagctagaaatagcaagttaaaataaggctagtc | 215 |
| term | cgttatc | |
| PBS_13_RT_ | aacttgaaaaagtggcaccgagtcggtgcGAGTCGGTGCGACGAGCGCGGC | |
| 29_with | GATATCATCATCCATGGagcatttaccttgattgagatgttaattgtgTGAGCTG | |
| TP901-1 | CGAGAA | |
| minimal | | |
| AttB rc | | |
| atgRNA | | |
| ACTB N- | GCTATTCTCGCAGCTCACCAgttttagagctagaaatagcaagttaaaataaggctagtc | 216 |
| term | cgttatc | |
| PBS_13_RT_ | aacttgaaaaagtggcaccgagtcggtgcGAGTCGGTGCGACGAGCGCGGC | |
| 29_with | GATATCATCATCCATGGcaggtttttgacgaaagtgatccagatgatccagTGAG | |
| PhiBT1 | CTGCGAGAA | |
| minimal | | |
| AttB f | | |
| atgRNA | | |
| ACTB N- | GCTATTCTCGCAGCTCACCAgttttagagctagaaatagcaagttaaaataaggctagtc | 217 |
| term | cgttatc | |
| PBS_13_RT_ | aacttgaaaaagtggcaccgagtcggtgcGAGTCGGTGCGACGAGCGCGGC | |
| 29_with | GATATCATCATCCATGGctggatcatctggatcactttcgtcaaaaacctgTGAGC | |
| PhiBT1 | TGCGAGAA | |
| minimal | | |
| AttB rc | | |
| atgRNA | | |
| ACTB N- | GAAGCCGGCCTTGCACATGCgttttagagctagaaatagcaagttaaaataaggctagtc | 218 |
| term | cgttat caacttgaaaaagtggcaccgagtcggtgc | |
| Nicking | | |
| guide 1 +48 | | |
| guide | | |
| ACTB N- | GAAGCCGGCCTTGCACATGCgttttagagctagaaatagcaagttaaaataaggctagtc | 219 |
| term | cgttatcaacttgaaaaagtggcaccgagtcggtgcATATCATCATCCATGGtaccgttc | |
| PBS_18_RT_ | gtatagcatacattatacgaagttatTGAGCTGCGAGAATAGCC | |
| 16_with_ | | |
| Lo x71_Cre | | |
| atgRNA | | |
| ACTB N- | GAAGCCGGCCTTGCACATGCgttttagagctagaaatagcaagttaaaataaggctagtc | 220 |
| term | cgttatcaacttgaaaaagtggcaccgagtcggtgcGACGAGCGCGGCGATATCAT | |
| PBS_13_RT_ | CATCCATGGtaccgttcgtatagcatacattatacgaagttatTGAGCTGCGAGAA | |
| 29_with_ | | |
| Lo x71_Cre | | |
| atgRNA | | |
| ACTB N- | GCTATTCTCGCAGCTCACCAgttttagagctagaaatagcaagttaaaataaggctagtc | 221 |
| term PBS | cgttatcaacttgaaaaagtggcaccgagtcggtgcTCGACGACGAGCGCGGCGAT | |
| 13 RT | ATCATCATCCATGGccggatgatcctgacgacggagaccgccgtcgtcgacaagccg | |
| 34 atgRNA | gccTGAGCTGCGAGAA | |
| ACTB N- | GCTATTCTCGCAGCTCACCAgttttagagctagaaatagcaagttaaaataaggctagtc | 222 |
| term PBS | cgttatcaacttgaaaaagtggcaccgagtcggtgcGAGCGCGGCGATATCATCAT | |
| 13 RT | CCATGGccggatgatcctgacgacggagaccgccgtcgtcgacaagccggccTGAGCTG | |
| 26 atgRNA | CGAGAA | |
| ACTB N- | GCTATTCTCGCAGCTCACCAgttttagagctagaaatagcaagttaaaataaggctagtc | 223 |
| term PBS | cgttatcaacttgaaaaagtggcaccgagtcggtgcCGCGGCGATATCATCATCCA | |
| 13 RT | TGGccggatgatcctgacgacggagaccgccgtcgtcgacaagccggccTGAGCTGCGA | |
| 23 atgRNA | GAA | |
| ACTB N- | GCTATTCTCGCAGCTCACCAgttttagagctagaaatagcaagttaaaataaggctagtc | 224 |
| term PBS | cgttatcaacttgaaaaagtggcaccgagtcggtgcGGCGATATCATCATCCATGGc | |
| 13 RT | cggatgatcctgacgacggagaccgccgtcgtcgacaagccggccTGAGCTGCGAGAA | |
| 20 atgRNA | | |
| ACTB N- | GCTATTCTCGCAGCTCACCAgttttagagctagaaatagcaagttaaaataaggctagtc | 225 |
| term PBS | cgttatcaacttgaaaaagtggcaccgagtcggtgcATATCATCATCCATGGccggatg | |
| 13 RT | atcctgacgacggagaccgccgtcgtcgacaagccggccTGAGCTGCGAGAA | |
| 16 atgRNA | | |
| ACTB N- | GCTATTCTCGCAGCTCACCAgttttagagctagaaatagcaagttaaaataaggctagtc | 226 |
| term PBS | cgttatcaacttgaaaaagtggcaccgagtcggtgcTCGACGACGAGCGCGGCGAT | |
| 18 RT | ATCATCATCCATGGccggatgatcctgacgacggagaccgccgtcgtcgacaagccg | |
| 34 atgRNA | gccTGAGCTGCGAGAATAGCC | |
| ACTB N- | GCTATTCTCGCAGCTCACCAgttttagagctagaaatagcaagttaaaataaggctagtc | 227 |
| term PBS | cgttatcaacttgaaaaagtggcaccgagtcggtgcGACGAGCGCGGCGATATCAT | |
| 18 RT | CATCCATGGccggatgatcctgacgacggagaccgccgtcgtcgacaagccggccTGA | |
| 29 atgRNA | GCTGCGAGAATAGCC | |
| ACTB N- | GCTATTCTCGCAGCTCACCAgttttagagctagaaatagcaagttaaaataaggctagtc | 228 |
| term PBS | cgttatcaacttgaaaaagtggcaccgagtcggtgcATATCATCATCCATGGccggatg | |
| 18 RT | atcctgacgacggagaccgccgtcgtcgacaagccggccTGAGCTGCGAGAATAGCC | |
| 16 atgRNA | | |
| LMNB1 N- | GCTGTCTCCGCCGCCCGCCAgttttagagctagaaatagcaagttaaaataaggctagtc | 229 |
| term PBS | cgttatcaacttgaaaaagtggcaccgagtcggtgcCTGCCCATCCGCGGCGGCAC | |
| 13 RT 39 | GGGGGTCGCAGTCGCCATGccggatgatcctgacgacggagaccgccgtcgtcg | |
| atgRNA | acaagccggccCGGGCGGCGGAGA | |
| LMNB1 N- | GCTGTCTCCGCCGCCCGCCAgttttagagctagaaatagcaagttaaaataaggctagtc | 230 |
| term PBS | cgttatcaacttgaaaaagtggcaccgagtcggtgcCATCCGCGGCGGCACGGGGG | |
| 13 RT 34 | TCGCAGTCGCCATGccggatgatcctgacgacggagaccgccgtcgtcgacaagccg | |
| atgRNA | gccCGGGCGGCGGAGA | |
| LMNB1 N- | GCTGTCTCCGCCGCCCGCCAgttttagagctagaaatagcaagttaaaataaggctagtc | 231 |
| term PBS | cgttatcaacttgaaaaagtggcaccgagtcggtgcGCGGCGGCACGGGGGTCGCA | |
| 13 RT 29 | GTCGCCATGccggatgatcctgacgacggagaccgccgtcgtcgacaagccggccCGG | |
| atgRNA | GCGGCGGAGA | |
| LMNB1 N- | GCTGTCTCCGCCGCCCGCCAgttttagagctagaaatagcaagttaaaataaggctagtc | 232 |
| term PBS | cgttatcaacttgaaaaagtggcaccgagtcggtgcGGCACGGGGGTCGCAGTCGC | |
| 13 RT 24 | CATGccggatgatcctgacgacggagaccgccgtcgtcgacaagccggccCGGGCGGC | |
| atgRNA | GGAGA | |
| LMNB1 N- | GCTGTCTCCGCCGCCCGCCAgttttagagctagaaatagcaagttaaaataaggctagtc | 233 |
| term PBS | cgttatcaacttgaaaaagtggcaccgagtcggtgcGGGGGTCGCAGTCGCCATGcc | |
| 13 RT 19 | ggatgatcctgacgacggagaccgccgtcgtcgacaagccggccCGGGCGGCGGAGA | |
| atgRNA | | |
| LMNB1 N- | GCTGTCTCCGCCGCCCGCCAgttttagagctagaaatagcaagttaaaataaggctagtc | 234 |
| term PBS | cgttatcaacttgaaaaagtggcaccgagtcggtgcCTGCCCATCCGCGGCGGCAC | |
| 18 RT 39 | GGGGGTCGCAGTCGCCATGccggatgatcctgacgacggagaccgccgtcgtcg | |
| atgRNA | acaagccggccCGGGCGGCGGAGACAGCG | |
| LMNB1 N- | GCTGTCTCCGCCGCCCGCCAgttttagagctagaaatagcaagttaaaataaggctagtc | 235 |
| term PBS | cgttatcaacttgaaaaagtggcaccgagtcggtgcCATCCGCGGCGGCACGGGGG | |
| 18 RT 34 | TCGCAGTCGCCATGccggatgatcctgacgacggagaccgccgtcgtcgacaagccg | |
| atgRNA | gccCGGGCGGCGGAGACAGCG | |
| LMNB1 N- | GCTGTCTCCGCCGCCCGCCAgttttagagctagaaatagcaagttaaaataaggctagtc | 236 |
| term PBS | cgttatcaacttgaaaaagtggcaccgagtcggtgcGCGGCGGCACGGGGGTCGCA | |
| 18 RT 29 | GTCGCCATGccggatgatcctgacgacggagaccgccgtcgtcgacaagccggccCGG | |
| atgRNA | GCGGCGGAGACAGCG | |
| LMNB1 N- | GCTGTCTCCGCCGCCCGCCAgttttagagctagaaatagcaagttaaaataaggctagtc | 237 |
| term PBS | cgttatcaacttgaaaaagtggcaccgagtcggtgcGGCACGGGGGTCGCAGTCGC | |
| 18 RT 24 | CATGccggatgatcctgacgacggagaccgccgtcgtcgacaagccggccCGGGCGGC | |
| atgRNA | GGAGACAGCG | |
| LMNB1 N- | GCTGTCTCCGCCGCCCGCCAgttttagagctagaaatagcaagttaaaataaggctagtc | 238 |
| term PBS | cgttatcaacttgaaaaagtggcaccgagtcggtgcGGGGGTCGCAGTCGCCATGcc | |
| 18 RT 19 | ggatgatcctgacgacggagaccgccgtcgtcgacaagccggccCGGGCGGCGGAGAC | |
| atgRNA | AGCG | |
| LMNB1 N- | GCGTGGTGGGGCCGCCAGCGgttttagagctagaaatagcaagttaaaataaggctagt | 239 |
| term | ccgttatcaacttgaaaaagtggcaccgagtcggtgc | |
| Nicking | | |
| guide 1 +46 | | |
| ACTB N- | GCTATTCTCGCAGCTCACCAgttttagagctagaaatagcaagttaaaataaggctagtc | 240 |
| term PBS | cgttatcaacttgaaaaagtggcaccgagtcggtgcGACGAGCGCGGCGATATCAT | |
| 13 RT | CATCCATGGggatgatcctgacgacggagaccgccgtcgtcgacaagccggTGAGCT | |
| 29 AttB 42 | GCGAGAA | |
| atgRNA | | |
| ACTB N- | GCTATTCTCGCAGCTCACCAgttttagagctagaaatagcaagttaaaataaggctagtc | 241 |
| term PBS | cgttatcaacttgaaaaagtggcaccgagtcggtgcGACGAGCGCGGCGATATCAT | |
| 13 RT | CATCCATGGgatgatcctgacgacggagaccgccgtcgtcgacaagccgTGAGCTGC | |
| 29 AttB 40 | GAGAA | |
| atgRNA | | |
| ACTB N- | GCTATTCTCGCAGCTCACCAgttttagagctagaaatagcaagttaaaataaggctagtc | 242 |
| term PBS | cgttatcaacttgaaaaagtggcaccgagtcggtgcGACGAGCGCGGCGATATCAT | |
| 13 RT | CATCCATGGatgatcctgacgacggagaccgccgtcgtcgacaagccTGAGCTGCG | |
| 29 AttB 38 | AGAA | |
| atgRNA | | |
| ACTB N- | GCTATTCTCGCAGCTCACCAgttttagagctagaaatagcaagttaaaataaggctagtc | 243 |
| term PBS | cgttatcaacttgaaaaagtggcaccgagtcggtgcGACGAGCGCGGCGATATCAT | |
| 13 RT | CATCCATGGtgatcctgacgacggagaccgccgtcgtcgacaagcTGAGCTGCGAG | |
| 29 AttB 36 | AA | |
| atgRNA | | |
| LMNB1 N- | GCTGTCTCCGCCGCCCGCCAgttttagagctagaaatagcaagttaaaataaggctagtc | 244 |
| term PBS | cgttatcaacttgaaaaagtggcaccgagtcggtgcGCGGCGGCACGGGGGTCGCA | |
| 13 | GTCGCCATGcggatgatcctgacgacggagaccgccgtcgtcgacaagccggcCGGG | |
| RT 29 AttB | CGGCGGAGA | |
| 44 atgRNA | | |
| v2 | | |
| LMNB1 N- | GCTGTCTCCGCCGCCCGCCAgttttagagctagaaatagcaagttaaaataaggctagtc | 245 |
| term PBS | cgttatcaacttgaaaaagtggcaccgagtcggtgcGCGGCGGCACGGGGGTCGCA | |
| 13 | GTCGCCATGggatgatcctgacgacggagaccgccgtcgtcgacaagccggCGGGCG | |
| RT 29 AttB | GCGGAGA | |
| 42 atgRNA | | |
| v2 | | |
| LMNB1 N- | GCTGTCTCCGCCGCCCGCCAgttttagagctagaaatagcaagttaaaataaggctagtc | 1246 |
| term PBS | cgttatcaacttgaaaaagtggcaccgagtcggtgcGCGGCGGCACGGGGGTCGCA | |
| 13 | GTCGCCATGgatgatcctgacgacggagaccgccgtcgtcgacaagccgCGGGCGG | |
| RT 29 AttB | CGGAGA | |
| 40 atgRNA | | |
| v2 | | |
| LMNB1 N- | GCTGTCTCCGCCGCCCGCCAgttttagagctagaaatagcaagttaaaataaggctagtc | 247 |
| term PBS | cgttatcaacttgaaaaagtggcaccgagtcggtgcGCGGCGGCACGGGGGTCGCA | |
| 13 | GTCGCCATGatgatcctgacgacggagaccgccgtcgtcgacaagccCGGGCGGC | |
| RT 29 AttB | GGAGA | |
| 38 atgRNA | | |
| v2 | | |
| NOLC1 N- | GCGTATTGCCTGGAGGATGGgttttagagctagaaatagcaagttaaaataaggctagtc | 248 |
| term PBS | cgttatcaacttgaaaaagtggcaccgagtcggtgcGAACCACGCGGCGAATGCCG | |
| 18 | GCGTCCGCCccggatgatcctgacgacggagaccgccgtcgtcgacaagccggccTCCT | |
| RT 29 AttB | CCAGGCAATACGCG | |
| 46 atgRNA | | |
| NOLC1 N- | GCGTATTGCCTGGAGGATGGgttttagagctagaaatagcaagttaaaataaggctagtc | 249 |
| term PBS | cgttatcaacttgaaaaagtggcaccgagtcggtgcGAACCACGCGGCGAATGCCG | |
| 13 | GCGTCCGCCccggatgatcctgacgacggagaccgccgtcgtcgacaagccggccTCCT | |
| RT 29 AttB | CCAGGCAAT | |
| 46 atgRNA | | |
| NOLC1 N- | GCGTATTGCCTGGAGGATGGgttttagagctagaaatagcaagttaaaataaggctagtc | 250 |
| term PBS | cgttatcaacttgaaaaagtggcaccgagtcggtgcGAACCACGCGGCGAATGCCG | |
| 13 | GCGTCCGCCcggatgatcctgacgacggagaccgccgtcgtcgacaagccggcTCCTC | |
| RT 29 AttB | CAGGCAAT | |
| 44 atgRNA | | |
| NOLC1 N- | GCGTATTGCCTGGAGGATGGgttttagagctagaaatagcaagttaaaataaggctagtc | 251 |
| term PBS | cgttatcaacttgaaaaagtggcaccgagtcggtgcGAACCACGCGGCGAATGCCG | |
| 13 | GCGTCCGCCggatgatcctgacgacggagaccgccgtcgtcgacaagccggTCCTCC | |
| RT 29 AttB | AGGCAAT | |
| 42 atgRNA | | |
| NOLC1 N- | GCGTATTGCCTGGAGGATGGgttttagagctagaaatagcaagttaaaataaggctagtc | 252 |
| term PBS | cgttatcaacttgaaaaagtggcaccgagtcggtgcGAACCACGCGGCGAATGCCG | |
| 13 | GCGTCCGCCgatgatcctgacgacggagaccgccgtcgtcgacaagccgTCCTCCAG | |
| RT 29 AttB | GCAAT | |
| 40 atgRNA | | |
| NOLC1 N- | GCGTATTGCCTGGAGGATGGgttttagagctagaaatagcaagttaaaataaggctagtc | 253 |
| term PBS | cgttatcaacttgaaaaagtggcaccgagtcggtgcGAACCACGCGGCGAATGCCG | |
| 13 | GCGTCCGCCatgatcctgacgacggagaccgccgtcgtcgacaagccTCCTCCAGG | |
| RT 29 AttB | CAAT | |
| 38 atgRNA | | |
| NOLC1 | GAGCCGAGCACGAGGGGATACgttttagagctagaaatagcaagttaaaataaggcta | 254 |
| nicking | gtccgttatcaacttgaaaaagtggcaccgagtcggtgc | |
| guide −43 | | |
| ACTB N- | GCTATTCTCGCAGCTCACCAgttttagagctagaaatagcaagttaaaataaggctagtc | 255 |
| term PBS | cgttatcaacttgaaaaagtggcaccgagtcggtgcGGCGATATCATCATCCATGGa | |
| 13 RT | tgatcctgacgacggagaccgccgtcgtcgacaagccTGAGCTGCGAGAA | |
| 20 AttB 38 | | |
| atgRNA | | |
| ACTB N- | GCTATTCTCGCAGCTCACCAgttttagagctagaaatagcaagttaaaataaggctagtc | 256 |
| term PBS | cgttatcaacttgaaaaagtggcaccgagtcggtgcTATCATCATCCATGGatgatcctga | |
| 13 RT | cgacggagaccgccgtcgtcgacaagccTGAGCTGCGAGAA | |
| 15 AttB 38 | | |
| atgRNA | | |
| ACTB N- | GCTATTCTCGCAGCTCACCAgttttagagctagaaatagcaagttaaaataaggctagtc | 257 |
| term PBS | cgttatcaacttgaaaaagtggcaccgagtcggtgcTCATCCATGGatgatcctgacgacgga | |
| 13 RT | gaccgccgtcgtcgacaagccTGAGCTGCGAGAA | |
| 10 AttB 38 | | |
| atgRNA | | |
| ACTB N- | GCTATTCTCGCAGCTCACCAgttttagagctagaaatagcaagttaaaataaggctagtc | 258 |
| term PBS 9 | cgttatcaacttgaaaaagtggcaccgagtcggtgcGGCGATATCATCATCCATGGa | |
| RT | tgatcctgacgacggagaccgccgtcgtcgacaagccTGAGCTGCG | |
| 20 AttB 38 | | |
| atgRNA | | |
| ACTB N- | GCTATTCTCGCAGCTCACCAgttttagagctagaaatagcaagttaaaataaggctagtc | 259 |
| term PBS 9 | cgttatcaacttgaaaaagtggcaccgagtcggtgcTATCATCATCCATGGatgatcctga | |
| RT | cgacggagaccgccgtcgtcgacaagccTGAGCTGCG | |
| 15 AttB 38 | | |
| atgRNA | | |
| ACTB N- | GCTATTCTCGCAGCTCACCAgttttagagctagaaatagcaagttaaaataaggctagtc | 260 |
| term PBS 9 | cgttatcaacttgaaaaagtggcaccgagtcggtgcTCATCCATGGatgatcctgacgacgga | |
| RT | gaccgccgtcgtcgacaagccTGAGCTGCG | |
| 10 AttB 38 | | |
| atgRNA | | |
| LMNB1 N- | GCTGTCTCCGCCGCCCGCCAgttttagagctagaaatagcaagttaaaataaggctagtc | 261 |
| term PBS | cgttatcaacttgaaaaagtggcaccgagtcggtgcCGGGGGTCGCAGTCGCCATG | |
| 13 | atgatcctgacgacggagaccgccgtcgtcgacaagccCGGGCGGCGGAGA | |
| RT 20 AttB | | |
| 38 atgRNA | | |
| LMNB1 N- | GCTGTCTCCGCCGCCCGCCAgttttagagctagaaatagcaagttaaaataaggctagtc | 262 |
| term PBS | cgttatcaacttgaaaaagtggcaccgagtcggtgcGTCGCAGTCGCCATGatgatcctg | |
| 13 | acgacggagaccgccgtcgtcgacaagccCGGGCGGCGGAGA | |
| RT 15 AttB | | |
| 38 atgRNA | | |
| LMNB1 N- | GCTGTCTCCGCCGCCCGCCAgttttagagctagaaatagcaagttaaaataaggctagtc | 263 |
| term PBS | cgttatcaacttgaaaaagtggcaccgagtcggtgcAGTCGCCATGatgatcctgacgacgga | |
| 13 | gaccgccgtcgtcgacaagccCGGGCGGCGGAGA | |
| RT 10 AttB | | |
| 38 atgRNA | | |
| LMNB1 N- | GCTGTCTCCGCCGCCCGCCAgttttagagctagaaatagcaagttaaaataaggctagtc | 264 |
| term PBS 9 | cgttatcaacttgaaaaagtggcaccgagtcggtgcCGGGGGTCGCAGTCGCCATG | |
| RT 20 AttB | atgatcctgacgacggagaccgccgtcgtcgacaagccCGGGCGGCG | |
| 38 atgRNA | | |
| LMNB1 N- | GCTGTCTCCGCCGCCCGCCAgttttagagctagaaatagcaagttaaaataaggctagtc | 265 |
| term PBS 9 | cgttatcaacttgaaaaagtggcaccgagtcggtgcGTCGCAGTCGCCATGatgatcctg | |
| RT 15 AttB | acgacggagaccgccgtcgtcgacaagccCGGGCGGCG | |
| 38 atgRNA | | |
| LMNB1 N- | GCTGTCTCCGCCGCCCGCCAgttttagagctagaaatagcaagttaaaataaggctagtc | 266 |
| term PBS 9 | cgttatcaacttgaaaaagtggcaccgagtcggtgcAGTCGCCATGatgatcctgacgacgga | |
| RT 10 AttB | gaccgccgtcgtcgacaagccCGGGCGGCG | |
| 38 atgRNA | | |
| SUPT16H | GAGAAGCGGCGTCCGGGGCTAgttttagagctagaaatagcaagttaaaataaggcta | 267 |
| N-term PBS | gtccgttatcaacttgaaaaagtggcaccgagtcggtgcTCTTTGTCCAGAGTCACAG | |
| 13 | CCATAccggatgatcctgacgacggagaccgccgtcgtcgacaagccggccCCCCGGAC | |
| RT 24 | GCCGC | |
| Bxb1- | | |
| GT_Initial | | |
| length | | |
| SRRM2 N- | GGGCACGGGGCCATGTACAAgttttagagctagaaatagcaagttaaaataaggctagt | 268 |
| term PBS | ccgttatcaacttgaaaaagtggcaccgagtcggtgcGGCGTCGGCAGCCCGATCC | |
| 13 | CGTTGccggatgatcctgacgacggagaccgccgtcgtcgacaagccggccTACATGGC | |
| RT 24 | CCCGT | |
| Bxb1 Initial | | |
| length | | |
| DEPDC4 | GTGTCAGGTGGGGCGGGGCTAgttttagagctagaaatagcaagttaaaataaggctag | 269 |
| N-term PBS | tccgttatcaacttgaaaaagtggcaccgagtcggtgcGCTGGCTCCTCCCCTGGCA | |
| 18 | CCATAccggatgatcctgacgacggagaccgccgtcgtcgacaagccggccCCCCGCCC | |
| RT 24 | CACCTGACAC | |
| Bxb1 Initial | | |
| length | | |
| NES N- | GAGTGGGTCAGACGAGCAGGAgttttagagctagaaatagcaagttaaaataaggcta | 270 |
| term PBS | gtccgttatcaacttgaaaaagtggcaccgagtcggtgcCGACTCCTCCCCCATGCAG | |
| 13 RT | CCCTCCATCccggatgatcctgacgacggagaccgccgtcgtcgacaagccggccTGCT | |
| 29 Bxb1 | CGTCTGACC | |
| Initial | | |
| length | | |
| SUPT16H | GCAGCCACCCGCTCTCGGCCCgttttagagctagaaatagcaagttaaaataaggctagt | 271 |
| nicking | ccgttatcaacttgaaaaagtggcaccgagtcggtgc | |
| guide −53 | | |
| SRRM2 N- | GTGTAGTCAGGCCGCTCACCCgttttagagctagaaatagcaagttaaaataaggctagt | 272 |
| term | ccgttatcaacttgaaaaagtggcaccgagtcggtgc | |
| nicking | | |
| guide 1 +87 | | |
| DEPDC4 | GCTGACAAGTCTACGGAACCTgttttagagctagaaatagcaagttaaaataaggctag | 273 |
| N-term | tccgttatcaacttgaaaaagtggcaccgagtcggtgc | |
| Nicking | | |
| guide 1 +59 | | |
| NES N- | GCTCCTCCAGCGCCTTGACCgttttagagctagaaatagcaagttaaaataaggctagtc | 274 |
| term | cgttatcaacttgaaaaagtggcaccgagtcggtgc | |
| Nicking | | |
| guide 2 +79 | | |
| HITI_ACT | GCTATTCTCGCAGCTCACCAgttttagagctagaaatagcaagttaaaataaggctagtc | 275 |
| B_guide | cgttatcaacttgaaaaagtggcaccgagtcggtgc | |
| HITI_SUP | AGAAGCGGCGTCCGGGGCTAgttttagagctagaaatagcaagttaaaataaggctagt | 276 |
| TH16_guide | ccgttatcaacttgaaaaagtggcaccgagtcggtgc | |
| HITI_SRR | GGGCACGGGGCCATGTACAAgttttagagctagaaatagcaagttaaaataaggctagt | 277 |
| M2_guide | ccgttatcaacttgaaaaagtggcaccgagtcggtgc | |
| HITI_NOL | GCGTATTGCCTGGAGGATGGgttttagagctagaaatagcaagttaaaataaggctagtc | 278 |
| Cl_guide | cgttatcaacttgaaaaagtggcaccgagtcggtgc | |
| HITI_DEP | TGTCAGGTGGGGCGGGGCTAgttttagagctagaaatagcaagttaaaataaggctagtc | 279 |
| DC4_guide | cgttatcaacttgaaaaagtggcaccgagtcggtgc | |
| HITI_NES_ | AGTGGGTCAGACGAGCAGGAgttttagagctagaaatagcaagttaaaataaggctagt | 280 |
| guide | ccgttatcaacttgaaaaagtggcaccgagtcggtgc | |
| HITI_LMN | GCTGTCTCCGCCGCCCGCCAgttttagagctagaaatagcaagttaaaataaggctagtc | 281 |
| B1_guide | cgttatcaacttgaaaaagtggcaccgagtcggtgc | |
| HDR Cas9 | GCTATTCTCGCAGCTCACCAgttttagagctagaaatagcaagttaaaataaggctagtc | 275 |
| ACTB | cgttatcaacttgaaaaagtggcaccgagtcggtgc | |
| guide | | |
| HDR Cas9 | GGGGTCGCAGTCGCCATGGCgttttagagctagaaatagcaagttaaaataaggctagtc | 282 |
| LMNB1 | cgttatcaacttgaaaaagtggcaccgagtcggtgc | |
| guide | | |
| ACTB N- | GCTATTCTCGCAGCTCACCAgttttagagctagaaatagcaagttaaaataaggctagtc | 283 |
| term PBS | cgttatcaacttgaaaaagtggcaccgagtcggtgcGACGAGCGCGGCGATATCAT | |
| 13 RT | CATCCATGGccggatgatcctgacgacggagXXcgccgtcgtcgacaagccggccTGA | |
| 29 AttB | GCTGCGAGAA | |
| original | XX: CG, GC, AT, TA, GG, TT, GA, AG, CC, TC, CT, AA, TG, GT, CA, AC | |
| length | | |
| atgRNAs | | |
| for | | |
| dinucleotides | | |
| ACTB N- | GCTATTCTCGCAGCTCACCAgttttagagctagaaatagcaagttaaaataaggctagtc | 284 |
| term PBS | cgttatcaacttgaaaaagtggcaccgagtcggtgcGACGAGCGCGGCGATATCAT | |
| 13 RT | CATCCATGccggatgatcctgacgacggagACcgccgtcgtcgacaagccggccTGAG | |
| 29 atgRNA | CTGCGAGAA | |
| with AttB | | |
| 46 GT for | | |
| fusion | | |
| ACTB N- | GCTATTCTCGCAGCTCACCAgttttagagctagaaatagcaagttaaaataaggctagtc | 285 |
| term PBS | cgttatcaacttgaaaaagtggcaccgagtcggtgcGACGAGCGCGGCGATATCAT | |
| 13 RT | CATCCATGccggatgatcctgacgacggagAGcgccgtcgtcgacaagccggccTGAG | |
| 29 atgRNA | CTGCGAGAA | |
| with AttB | | |
| 46 CT for | | |
| multiplexing | | |
| NOLC1N- | GCGTATTGCCTGGAGGATGGgttttagagctagaaatagcaagttaaaataaggctagtc | 286 |
| term PBS | cgttatcaacttgaaaaagtggcaccgagtcggtgcGAACCACGCGGCGAATGCCG | |
| 18 | GCGTCCGCCccggatgatcctgacgacggagTCcgccgtcgtcgacaagccggccTCC | |
| RT 29 | TCCAGGCAATACGCG | |
| atgRNA | | |
| with AttB | | |
| 46 GA for | | |
| multiplexing | | |
| LMNB1 N- | GCTGTCTCCGCCGCCCGCCAgttttagagctagaaatagcaagttaaaataaggctagtc | 287 |
| term PBS | cgttatcaacttgaaaaagtggcaccgagtcggtgcGCGGCGGCACGGGGGTCGCA | |
| 18 | GTCGCCATGccggatgatcctgacgacggagCTcgccgtcgtcgacaagccggccCG | |
| RT 29 | GGCGGCGGAGACAGCG | |
| atgRNA | | |
| with AttB | | |
| 46 AG for | | |
| multiplexing | | |
| EMX1 | GTCACCTCCAATGACTAGGGgttttagagctagaaatagcaagttaaaataaggctagtc | 288 |
| Cas9 guide 1 | cgttatcaacttgaaaaagtggcaccgagtcggtgc | |
| EMX1 | GGGCAACCACAAACCCACGAgttttagagctagaaatagcaagttaaaataaggctagt | 289 |
| Cas9 guide 2 | ccgttatcaacttgaaaaagtggcaccgagtcggtgc | |
| ACTB N- | GCTATTCTCGCAGCTCACCAgttttagagctagaaatagcaagttaaaataaggctagtc | 290 |
| term PBS | cgttatcaacttgaaaaagtggcaccgagtcggtgcGACGAGCGCGGCGATATCAT | |
| 13 RT | CATCCATGGctatgccggatgatcctgacgacggagtccgccgtcgtcgacaagccggccc | |
| 29 AttB 56 | tagcTGAGCTGCGAGAA | |
| GA | | |
| atgRNA | | |
| ACTB N- | GCTATTCTCGCAGCTCACCAgttttagagctagaaatagcaagttaaaataaggctagtc | 291 |
| term PBS | cgttatcaacttgaaaaagtggcaccgagtcggtgcGACGAGCGCGGCGATATCAT | |
| 13 RT | CATCCATGGtgccggatgatcctgacgacggagtccgccgtcgtcgacaagccggccctaT | |
| 29 AttB 51 | GAGCTGCGAGAA | |
| GA | | |
| atgRNA | | |
| ACTB N- | GCTATTCTCGCAGCTCACCAgttttagagctagaaatagcaagttaaaataaggctagtc | 292 |
| term PBS | cgttatcaacttgaaaaagtggcaccgagtcggtgcGACGAGCGCGGCGATATCAT | |
| 13 RT | CATCCATGGccggatgatcctgacgacggagtccgccgtcgtcgacaagccggccTGAG | |
| 29 AttB 46 | CTGCGAGAA | |
| GA | | |
| atgRNA | | |
| ACTB N- | GCTATTCTCGCAGCTCACCAgttttagagctagaaatagcaagttaaaataaggctagtc | 293 |
| term PBS | cgttatcaacttgaaaaagtggcaccgagtcggtgcGACGAGCGCGGCGATATCATC | |
| 13 RT | ATCCATGGggatgatcctgacgacggagtccgccgtcgtcgacaagccgTGAGCTGCG | |
| 29 AttB 41 | AGAA | |
| GA | | |
| atgRNA | | |
| ACTB N- | GCTATTCTCGCAGCTCACCAgttttagagctagaaatagcaagttaaaataaggctagtc | 294 |
| term PBS | cgttatcaacttgaaaaagtggcaccgagtcggtgcGACGAGCGCGGCGATATCAT | |
| 13 RT | CATCCATGGtgatcctgacgacggagtccgccgtcgtcgacaagcTGAGCTGCGAG | |
| 29 AttB 36 | AA | |
| GA | | |
| atgRNA | | |
| ACTB N- | GCTATTCTCGCAGCTCACCAgttttagagctagaaatagcaagttaaaataaggctagtc | 295 |
| term PBS | cgttatcaacttgaaaaagtggcaccgagtcggtgcGACGAGCGCGGCGATATCAT | |
| 13 RT | CATCCATGGatcctgacgacggagtccgccgtcgtcgacaTGAGCTGCGAGAA | |
| 29 AttB 31 | | |
| GA | | |
| atgRNA | | |
| ACTB N- | GCTATTCTCGCAGCTCACCAgttttagagctagaaatagcaagttaaaataaggctagtc | 296 |
| term PBS | cgttatcaacttgaaaaagtggcaccgagtcggtgcGACGAGCGCGGCGATATCAT | |
| 13 RT | CATCCATGGcctgacgacggagtccgccgtcgtcgTGAGCTGCGAGAA | |
| 29 AttB 26 | | |
| GA | | |
| atgRNA | | |
| ACTB N- | GCTATTCTCGCAGCTCACCAgttttagagctagaaatagcaagttaaaataaggctagtc | 297 |
| term PBS | cgttatcaacttgaaaaagtggcaccgagtcggtgcGACGAGCGCGGCGATATCATC | |
| 13 RT | ATCCATGGtgacgacggagtccgccgtcgTGAGCTGCGAGAA | |
| 29 AttB 21 | | |
| GA | | |
| atgRNA | | |
| ACTB N- | GCTATTCTCGCAGCTCACCAgttttagagctagaaatagcaagttaaaataaggctagtc | 298 |
| term PBS | cgttatcaacttgaaaaagtggcaccgagtcggtgcGACGAGCGCGGCGATATCAT | |
| 13 RT | CATCCATGGacgacggagtccgccgTGAGCTGCGAGAA | |
| 29 AttB 16 | | |
| GA | | |
| atgRNA | | |
| ACTB N- | GCTATTCTCGCAGCTCACCAgttttagagctagaaatagcaagttaaaataaggctagtc | 299 |
| term PBS | cgttatcaacttgaaaaagtggcaccgagtcggtgcGACGAGCGCGGCGATATCAT | |
| 13 RT | CATCCATGGgacggagtccgTGAGCTGCGAGAA | |
| 29 AttB 11 | | |
| GA | | |
| atgRNA | | |
| ACTB N- | GCTATTCTCGCAGCTCACCAgttttagagctagaaatagcaagttaaaataaggctagtc | 300 |
| term PBS | cgttatcaacttgaaaaagtggcaccgagtcggtgcGACGAGCGCGGCGATATCAT | |
| 13 RT | CATCCATGGcggagtTGAGCTGCGAGAA | |
| 29 AttB 6 | | |
| GA | | |
| atgRNA | | |
| ACTB N- | GAAGCCGGCCTTGCACATGCgttttagagctagaaatagcaagttaaaataaggctagtc | 301 |
| term | cgttatcaacttgaaaaagtggcaccgagtcggtgcTCGACGACGAGCGCGGCGAT | |
| PBS_18_RT_ | ATCATCATCCATGGtaccgttcgtatagcatacattatacgaagttatTGAGCTGC | |
| 34_with_ | GAGAATAGCC | |
| Lo_x71_Cre | | |
| atgRNA | | |
| ACTB N- | GAAGCCGGCCTTGCACATGCgttttagagctagaaatagcaagttaaaataaggctagtc | 302 |
| term | cgttatcaacttgaaaaagtggcaccgagtcggtgcGACGAGCGCGGCGATATCAT | |
| PBS_18_RT_ | CATCCATGGtaccgttcgtatagcatacattatacgaagttatTGAGCTGCGAGAAT | |
| 29_with | AGCC | |
| Lo_x71_Cre | | |
| atgRNA | | |
| ACTB N- | GAAGCCGGCCTTGCACATGCgttttagagctagaaatagcaagttaaaataaggctagtc | 303 |
| term | cgttatcaacttgaaaaagtggcaccgagtcggtgcTCGACGACGAGCGCGGCGAT | |
| PBS_13_RT_ | ATCATCATCCATGGtaccgttcgtatagcatacattatacgaagttatTGAGCTGC | |
| 34_with_ | GAGAA | |
| Lo_x71_Cre | | |
| atgRNA | | |
| ACTB N- | GAAGCCGGCCTTGCACATGCgttttagagctagaaatagcaagttaaaataaggctagtc | 304 |
| term | cgttatcaacttgaaaaagtggcaccgagtcggtgcATATCATCATCCATGGtaccgttc | |
| PBS_13_RT_ | gtatagcatacattatacgaagttatTGAGCTGCGAGAA | |
| 16_with_ | | |
| Lo_x71_Cre | | |
| atgRNA | | |
| ACTB N- | CCCCACGATGGAGGGGAAGAgttttagagctagaaatagcaagttaaaataaggctagt | 305 |
| term | ccgttatcaacttgaaaaagtggcaccgagtcggtgc | |
| Nicking | | |
| guide 2 +93 | | |
| guide | | |
| LMNB1 N- | CCTTCTCCTGGAGCCGCGACgttttagagctagaaatagcaagttaaaataaggctagtc | 306 |
| term | cgttatcaacttgaaaaagtggcaccgagtcggtgc | |
| Nicking | | |
| guide 2 +87 | | |
| guide | | |
| ACTB N- | GCTATTCTCGCAGCTCACCAgttttagagctagaaatagcaagttaaaataaggctagtc | 307 |
| term PBS | cgttatcaacttgaaaaagtggcaccgagtcggtgcGACGAGCGCGGCGATATCAT | |
| 13 RT 29 | CATCCATGGcattatatgttcttacagtatggcggcccggattgtaaaaacatataatgTGA | |
| AttB 46 | GCTGCGAGAA | |
| N191352_ | | |
| 143_72 | | |
| integrase | | |
| ACTB N- | GCTATTCTCGCAGCTCACCAgttttagagctagaaatagcaagttaaaataaggctagtc | 308 |
| term PBS | cgttatcaacttgaaaaagtggcaccgagtcggtgcGACGAGCGCGGCGATATCAT | |
| 13 RT | CATCCATGGcgttatagggtattacagtatggcggtcggtactgcaataccctataacgTG | |
| 29 AttB 46 | AGCTGCGAGAA | |
| N684346_ | | |
| 90_69 | | |
| integrase | | |
| ACTB N- | GCTATTCTCGCAGCTCACCAgttttagagctagaaatagcaagttaaaataaggctagtc | 309 |
| term PBS | cgttatcaacttgaaaaagtggcaccgagtcggtgcGACGAGCGCGGCGATATCAT | |
| 13 RT 29 | CATCCATGGtgtatcattttcatatagttagcacctgcacactatatgaaaatgatacaTGA | |
| AttB 46 | GCTGCGAGAA | |
| N675015_ | | |
| 95_5 | | |
| integrase | | |
| ACTB N- | GCTATTCTCGCAGCTCACCAgttttagagctagaaatagcaagttaaaataaggctagtc | 310 |
| term PBS | cgttatcaacttgaaaaagtggcaccgagtcggtgcGACGAGCGCGGCGATATCAT | |
| 13 RT 29 | CATCCATGGtgtctactatctgtatatgcgacacatgtggcataaagacatagtagacaTG | |
| AttB 46 | AGCTGCGAGAA | |
| N189929_ | | |
| 49_54 | | |
| integrase | | |
| ACTB N- | GCTATTCTCGCAGCTCACCAgttttagagctagaaatagcaagttaaaataaggctagtc | 311 |
| term PBS | cgttatcaacttgaaaaagtggcaccgagtcggtgcGACGAGCGCGGCGATATCAT | |
| 13 RT 29 | CATCCATGGcatcgaccctgacgcatgcggaggcggcgctccatgcgtctgacctcattTG | |
| AttB 46 | AGCTGCGAGAA | |
| N203911_ | | |
| 45186_6 | | |
| integrase | | |
| ACTB N- | GCTATTCTCGCAGCTCACCAgttttagagctagaaatagcaagttaaaataaggctagtc | 312 |
| term PBS | cgttatcaacttgaaaaagtggcaccgagtcggtgcGACGAGCGCGGCGATATCAT | |
| 13 RT 29 | CATCCATGGgttagtacccaaatgacaaaaggtcatccttttatcatttgggtactaacTGA | |
| AttB 46 | GCTGCGAGAA | |
| N687663_ | | |
| 53_29 | | |
| integrase | | |
| ACTB N- | GCTATTCTCGCAGCTCACCAgttttagagctagaaatagcaagttaaaataaggctagtc | 313 |
| term PBS | cgttatcaacttgaaaaagtggcaccgagtcggtgcGACGAGCGCGGCGATATCAT | |
| 13 RT 29 | CATCCATGGcttattaaaacccgttccgcttctgtcaaagcggcatcggttttataaacTGA | |
| AttB 46 | GCTGCGAGAA | |
| N687611 9 | | |
| 0 68 | | |
| integrase | | |
| ACTB N- | GCTATTCTCGCAGCTCACCAgttttagagctagaaatagcaagttaaaataaggctagtc | 314 |
| term PBS | cgttatcaacttgaaaaagtggcaccgagtcggtgcGACGAGCGCGGCGATATCAT | |
| 13 RT 29 | CATCCATGGggcgtgatggtcgtgaacctcaacatgacgacgaacacgacctcgcggccT | |
| AttB 46 | GAGCTGCGAGAA | |
| N190156_ | | |
| 234_12 | | |
| integrase | | |
| ACTB N- | GCTATTCTCGCAGCTCACCAgttttagagctagaaatagcaagttaaaataaggctagtc | 315 |
| term PBS | cgttatcaacttgaaaaagtggcaccgagtcggtgcGACGAGCGCGGCGATATCAT | |
| 13 RT 29 | CATCCATGGtctacatcttgaatatatcaagttataactttgaattatatcagtttataTGAG | |
| AttB 46 | CTGCGAGAA | |
| N191533_ | | |
| 224_76 | | |
| integrase | | |
| ACTB N- | GCTATTCTCGCAGCTCACCAgttttagagctagaaatagcaagttaaaataaggctagtc | 316 |
| term PBS | cgttatcaacttgaaaaagtggcaccgagtcggtgcGACGAGCGCGGCGATATCAT | |
| 13 RT 29 | CATCCATGGaattatatctaaaagcactaagctccgccatactgcttttagatataataTGA | |
| AttB 46 | GCTGCGAGAA | |
| N208621_ | | |
| 9_15 | | |
| integrase | | |
| ACTB N- | GCTATTCTCGCAGCTCACCAgttttagagctagaaatagcaagttaaaataaggctagtc | 317 |
| term PBS | cgttatcaacttgaaaaagtggcaccgagtcggtgcGACGAGCGCGGCGATATCAT | |
| 13 RT 29 | CATCCATGGgatatggggaagtgaatcagtacaaccgccacagtaccTGAGCTGCG | |
| AttB 46 | AGAA | |
| Bacillus_ | | |
| cereus_ | | |
| Ah187_38 | | |
| bp_Att | | |
| ACTB N- | GCTATTCTCGCAGCTCACCAgttttagagctagaaatagcaagttaaaataaggctagtc | 318 |
| term PBS | cgttatcaacttgaaaaagtggcaccgagtcggtgcGACGAGCGCGGCGATATCAT | |
| 13 RT 29 | CATCCATGGggtactgtggcggttgtactgattcacttccccatatcTGAGCTGCGAG | |
| AttB 46 | AA | |
| Bacillus_ | | |
| cereus_ | | |
| AH187_38 | | |
| bp_Att_rc | | |
| ACTB N- | GCTATTCTCGCAGCTCACCAgttttagagctagaaatagcaagttaaaataaggctagtc | 319 |
| term PBS | cgttatcaacttgaaaaagtggcaccgagtcggtgcGACGAGCGCGGCGATATCAT | |
| 13 RT 29 | CATCCATGGtgggtggtacaggtgccacattagttgtaccatttatgTGAGCTGCGAG | |
| AttB 46 | AA | |
| Staphylo- | | |
| coccus_ | | |
| lugdunensis_ | | |
| N920143_ | | |
| 38 bp_Att | | |
| ACTB N- | GCTATTCTCGCAGCTCACCAgttttagagctagaaatagcaagttaaaataaggctagtc | 320 |
| term PBS | cgttatcaacttgaaaaagtggcaccgagtcggtgcGACGAGCGCGGCGATATCAT | |
| 13 RT 29 | CATCCATGGcataaatggtacaactaatgtggcacctgtaccacccaTGAGCTGCGA | |
| AttB 46 | GAA | |
| Staphylo- | | |
| coccus_ | | |
| lugdunensis_ | | |
| N920143_ | | |
| 38 bp_Att_rc | | |
| ACTB N- | GCTATTCTCGCAGCTCACCAgttttagagctagaaatagcaagttaaaataaggctagtc | 321 |
| term PBS | cgttatcaacttgaaaaagtggcaccgagtcggtgcGACGAGCGCGGCGATATCAT | |
| 13 RT 29 | CATCCATGGgttgtttttccagatccagttggtcctgtaaatataagTGAGCTGCGAG | |
| AttB 46 | AA | |
| Bacillus_ | | |
| cytotoxicus_ | | |
| NVH_391- | | |
| 98_38 bp_Att | | |
| ACTB N- | GCTATTCTCGCAGCTCACCAgttttagagctagaaatagcaagttaaaataaggctagtc | 322 |
| term PBS | cgttatcaacttgaaaaagtggcaccgagtcggtgcGACGAGCGCGGCGATATCAT | |
| 13 RT 29 | CATCCATGGcttatatttacaggaccaactggatctggaaaaacaacTGAGCTGCGA | |
| AttB 46 | GAA | |
| Bacillus_ | | |
| cytotoxicus_ | | |
| NVH_391- | | |
| 98_38 bp_ | | |
| Att_rc | | |
| ACTB N- | GCTATTCTCGCAGCTCACCAgttttagagctagaaatagcaagttaaaataaggctagtc | 323 |
| term PBS | cgttatcaacttgaaaaagtggcaccgagtcggtgcGACGAGCGCGGCGATATCAT | |
| 13 RT 29 | CATCCATGGgtactgtggcggttgtactgattcacttccccatatTGAGCTGCGAGA | |
| AttB 46 | A | |
| Bacillus_ | | |
| cereus_AH18 | | |
| 7_Att 36 bp | | |
| ACTB N- | GCTATTCTCGCAGCTCACCAgttttagagctagaaatagcaagttaaaataaggctagtc | 324 |
| term PBS | cgttatcaacttgaaaaagtggcaccgagtcggtgcGACGAGCGCGGCGATATCAT | |
| 13 RT 29 | CATCCATGGtactgtggcggttgtactgattcacttccccataTGAGCTGCGAGAA | |
| AttB 46 | | |
| Bacillus_ | | |
| cereus_ | | |
| AH187_Att_ | | |
| 34 bp | | |
| ACTB N- | GCTATTCTCGCAGCTCACCAgttttagagctagaaatagcaagttaaaataaggctagtc | 325 |
| term PBS | cgttatcaacttgaaaaagtggcaccgagtcggtgcGACGAGCGCGGCGATATCAT | |
| 13 RT 29 | CATCCATGGactgtggcggttgtactgattcacttccccatTGAGCTGCGAGAA | |
| AttB 46 | | |
| Bacillus_ | | |
| cereus_ | | |
| AH187_Att_ | | |
| 32 bp | | |
| ACTB N- | GCTATTCTCGCAGCTCACCAgttttagagctagaaatagcaagttaaaataaggctagtc | 326 |
| term PBS | cgttatcaacttgaaaaagtggcaccgagtcggtgcGACGAGCGCGGCGATATCAT | |
| 13 RT 29 | CATCCATGGatatggggaagtgaatcagtacaaccgccacagtacTGAGCTGCGA | |
| AttB 46 | GAA | |
| Bacillus_ | | |
| cereus_ | | |
| AH187_Att_ | | |
| 36 bp | | |
| ACTB N- | GCTATTCTCGCAGCTCACCAgttttagagctagaaatagcaagttaaaataaggctagtc | 327 |
| term PBS | cgttatcaacttgaaaaagtggcaccgagtcggtgcGACGAGCGCGGCGATATCAT | |
| 13 RT 29 | CATCCATGGtatggggaagtgaatcagtacaaccgccacagtaTGAGCTGCGAGA | |
| AttB 46 | A | |
| Bacillus_ | | |
| cereus_ | | |
| AH187_Att_ | | |
| 34 bp | | |
| ACTB N- | GCTATTCTCGCAGCTCACCAgttttagagctagaaatagcaagttaaaataaggctagtc | 328 |
| term PBS | cgttatcaacttgaaaaagtggcaccgagtcggtgcGACGAGCGCGGCGATATCAT | |
| 13 RT 29 | CATCCATGGatggggaagtgaatcagtacaaccgccacagtTGAGCTGCGAGAA | |
| AttB 46 | | |
| Bacillus_ | | |
| cereus_ | | |
| AH187_Att_ | | |
| 32 bp | | |
| ACTB N- | GCTATTCTCGCAGCTCACCAgttttagagctagaaatagcaagttaaaataaggctagtc | 329 |
| term PBS | cgttatcaacttgaaaaagtggcaccgagtcggtgcGACGAGCGCGGCGATATCAT | |
| 13 RT 29 | CATCCATGGataaatggtacaactaatgtggcacctgtaccacccTGAGCTGCGAG | |
| AttB 46 | AA | |
| Staphylo- | | |
| coccus_ | | |
| lugdunensis_ | | |
| N920143_ | | |
| Att 36 bp | | |
| ACTB N- | GCTATTCTCGCAGCTCACCAgttttagagctagaaatagcaagttaaaataaggctagtc | 330 |
| term PBS | cgttatcaacttgaaaaagtggcaccgagtcggtgcGACGAGCGCGGCGATATCAT | |
| 13 RT 29 | CATCCATGGtaaatggtacaactaatgtggcacctgtaccaccTGAGCTGCGAGAA | |
| AttB 46 | | |
| Staphylo- | | |
| coccus_ | | |
| lugdunensis_ | | |
| N920143_ | | |
| Att 34 bp | | |
| ACTB N- | GCTATTCTCGCAGCTCACCAgttttagagctagaaatagcaagttaaaataaggctagtc | 331 |
| term PBS | cgttatcaacttgaaaaagtggcaccgagtcggtgcGACGAGCGCGGCGATATCAT | |
| 13 RT 29 | CATCCATGGaaatggtacaactaatgtggcacctgtaccacTGAGCTGCGAGAA | |
| AttB 46 | | |
| Staphylo- | | |
| coccus_ | | |
| lugdunensis_ | | |
| N920143_ | | |
| Att 32 bp | | |
| ACTB N- | GCTATTCTCGCAGCTCACCAgttttagagctagaaatagcaagttaaaataaggctagtc | 332 |
| term PBS | cgttatcaacttgaaaaagtggcaccgagtcggtgcGACGAGCGCGGCGATATCAT | |
| 13 RT 29 | CATCCATGGgggtggtacaggtgccacattagttgtaccatttatTGAGCTGCGAGA | |
| AttB 46 | A | |
| Staphylo- | | |
| coccus_ | | |
| lugdunensis_ | | |
| N920143 | | |
| Att_rc 36 bp | | |
| ACTB N- | GCTATTCTCGCAGCTCACCAgttttagagctagaaatagcaagttaaaataaggctagtc | 333 |
| term PBS | cgttatcaacttgaaaaagtggcaccgagtcggtgcGACGAGCGCGGCGATATCAT | |
| 13 RT 29 | CATCCATGGggtggtacaggtgccacattagttgtaccatttaTGAGCTGCGAGAA | |
| AttB 46 | | |
| Staphylo- | | |
| coccus_ | | |
| lugdunensis_ | | |
| N920143_ | | |
| Att_rc 34 bp | | |
| ACTB N- | GCTATTCTCGCAGCTCACCAgttttagagctagaaatagcaagttaaaataaggctagtc | 334 |
| term PBS | cgttatcaacttgaaaaagtggcaccgagtcggtgcGACGAGCGCGGCGATATCAT | |
| 13 RT 29 | CATCCATGGgtggtacaggtgccacattagttgtaccatttTGAGCTGCGAGAA | |
| AttB 46 | | |
| Staphylo- | | |
| coccus_ | | |
| lugdunensis_ | | |
| N920143_ | | |
| Att_rc 32 bp | | |
| ACTB N- | GCTATTCTCGCAGCTCACCAgttttagagctagaaatagcaagttaaaataaggctagtc | 335 |
| term PBS | cgttatcaacttgaaaaagtggcaccgagtcggtgcGACGAGCGCGGCGATATCAT | |
| 13 RT 29 | CATCCATGGttatatttacaggaccaactggatctggaaaaacaaTGAGCTGCGAG | |
| AttB 46 | AA | |
| Bacillus_ | | |
| cytotoxicus_ | | |
| NVH_391- | | |
| 98_Att | | |
| 36 bp | | |
| ACTB N- | GCTATTCTCGCAGCTCACCAgttttagagctagaaatagcaagttaaaataaggctagtc | 336 |
| term PBS | cgttatcaacttgaaaaagtggcaccgagtcggtgcGACGAGCGCGGCGATATCAT | |
| 13 RT 29 | CATCCATGGtatatttacaggaccaactggatctggaaaaacaTGAGCTGCGAGA | |
| AttB 46 | A | |
| Bacillus_ | | |
| cytotoxicus_ | | |
| NVH_391- | | |
| 98_Att | | |
| 34 bp | | |
| ACTB N- | GCTATTCTCGCAGCTCACCAgttttagagctagaaatagcaagttaaaataaggctagtc | 337 |
| term PBS | cgttatcaacttgaaaaagtggcaccgagtcggtgcGACGAGCGCGGCGATATCAT | |
| 13 RT 29 | CATCCATGGatatttacaggaccaactggatctggaaaaacTGAGCTGCGAGAA | |
| AttB 46 | | |
| Bacillus_ | | |
| cytotoxicus_ | | |
| NVH_391- | | |
| 98_Att | | |
| 32 bp | | |
| ACTB N- | GCTATTCTCGCAGCTCACCAgttttagagctagaaatagcaagttaaaataaggctagtc | 338 |
| term PBS | cgttatcaacttgaaaaagtggcaccgagtcggtgcGACGAGCGCGGCGATATCAT | |
| 13 RT 29 | CATCCATGGttgtttttccagatccagttggtcctgtaaatataaTGAGCTGCGAGAA | |
| AttB 46 | | |
| Bacillus_ | | |
| cytotoxicus_ | | |
| NVH_391- | | |
| 98_Att_rc | | |
| 36 bp | | |
| ACTB N- | GCTATTCTCGCAGCTCACCAgttttagagctagaaatagcaagttaaaataaggctagtc | 339 |
| term PBS | cgttatcaacttgaaaaagtggcaccgagtcggtgcGACGAGCGCGGCGATATCAT | |
| 13 RT 29 | CATCCATGGtgtttttccagatccagttggtcctgtaaatataTGAGCTGCGAGAA | |
| AttB 46 | | |
| Bacillus_ | | |
| cytotoxicus_ | | |
| NVH_391- | | |
| 98_Att_rc | | |
| 34 bp | | |
| ACTB N- | GCTATTCTCGCAGCTCACCAgttttagagctagaaatagcaagttaaaataaggctagtc | 340 |
| term PBS | cgttatcaacttgaaaaagtggcaccgagtcggtgcGACGAGCGCGGCGATATCAT | |
| 13 RT 29 | CATCCATGGgtttttccagatccagttggtcctgtaaatatTGAGCTGCGAGAA | |
| AttB 46 | | |
| Bacillus_ | | |
| cytotoxicus_ | | |
| NVH_391- | | |
| 98_Att_rc | | |
| 32 bp | | |
| Bacillus_ | GCTGTCTCCGCCGCCCGCCAgttttagagctagaaatagcaagttaaaataaggctagtc | 341 |
| cereus_AH18 | cgttatcaacttgaaaaagtggcaccgagtcggtgcAGTCGCCATGatatggggaagtgaatc | |
| 7 Att_rc_36 | agtacaaccgccacagtacCGGGCGGCG | |
| LMNB1 | | |
| PBS 9 RT | | |
| 10 AttB 36 | | |
| atgRNA | | |
| Bacillus_ | GCGTATTGCCTGGAGGATGGgttttagagctagaaatagcaagttaaaataaggctagtc | 342 |
| cereus_ | cgttatcaacttgaaaaagtggcaccgagtcggtgcGAACCACGCGGCGAATGCCG | |
| AH187_Att_ | GCGTCCGCCatatggggaagtgaatcagtacaaccgccacagtacTCCTCCAGGCA | |
| rc_36 | ATACGCG | |
| NOLC1 | | |
| PBS 18 RT | | |
| 29 AttB 36 | | |
| atgRNA | | |
| Bacillus_ | GAGAAGCGGCGTCCGGGGCTAgttttagagctagaaatagcaagttaaaataaggcta | 343 |
| cereus_ | gtccgttatcaacttgaaaaagtggcaccgagtcggtgcTCTTTGTCCAGAGTCACAG | |
| AH187_Att_ | CCATAatatggggaagtgaatcagtacaaccgccacagtacCCCCGGACGCCGC | |
| rc_36 | | |
| SUPT16H | | |
| PBS 13 | | |
| RT 24 AttB | | |
| 36 atgRNA | | |
| Bacillus_ | GGGCACGGGGCCATGTACAAgttttagagctagaaatagcaagttaaaataaggctagt | 344 |
| cereus_ | ccgttatcaacttgaaaaagtggcaccgagtcggtgcGGCGTCGGCAGCCCGATCC | |
| AH187_Att_ | CGTTGatatggggaagtgaatcagtacaaccgccacagtacTACATGGCCCCGT | |
| rc_36 | | |
| SRRM2 | | |
| PBS 13 RT | | |
| 24 AttB 36 | | |
| atgRNA | | |
| Bacillus_ | GTGTCAGGTGGGGCGGGGCTAgttttagagctagaaatagcaagttaaaataaggctag | 345 |
| cereus_ | tccgttatcaacttgaaaaagtggcaccgagtcggtgcGCTGGCTCCTCCCCTGGCA | |
| AH187_Att_ | CCATAatatggggaagtgaatcagtacaaccgccacagtacCCCCGCCCCACCTGA | |
| rc_36 | CAC | |
| DEPDC4 | | |
| PBS 18 | | |
| RT 24 AttB | | |
| 36 atgRNA | | |
| Bacillus_ | GAGTGGGTCAGACGAGCAGGAgttttagagctagaaatagcaagttaaaataaggcta | 346 |
| cereus_ | gtccgttatcaacttgaaaaagtggcaccgagtcggtgcCGACTCCTCCCCCATGCAG | |
| AH187_Att_ | CCCTCCATCatatggggaagtgaatcagtacaaccgccacagtacTGCTCGTCTGA | |
| rc_36 NES | CC | |
| PBS 13 RT | | |
| 28 | | |
| AttB 36 | | |
| atgRNA | | |
| B. cereus | GCTGTCTCCGCCGCCCGCCAgttttagagctagaaatagcaagttaaaataaggctagtc | 347 |
| LMNB1_ | cgttatca | |
| PBS 9 | acttgaaaaagtggcaccgagtcggtgcCGGGGGTCGCAGTCGCCATGatatggg | |
| RT 20 AttB | gaagtgaatcagtacaaccgccacagtacCGGGCGGCG | |
| 36 atgRNA | | |
| B. cereus | GGGCACGGGGCCATGTACAAgttttagagctagaaatagcaagttaaaataaggctagt | 348 |
| LMNB1_ | ccgttatcaacttgaaaaagtggcaccgagtcggtgcCGGGGGTCGCAGTCGCCATGat | |
| PBS 13 RT | atggggaagtgaatcagtacaaccgccacagtacCGGGCGGCGGAGA | |
| 20 AttB 36 | | |
| atgRNA | | |
| B. cereus | GGGCACGGGGCCATGTACAAgttttagagctagaaatagcaagttaaaataaggctagt | 349 |
| LMNB1_ | ccgttatcaacttgaaaaagtggcaccgagtcggtgcGCGGCGGCACGGGGGTCGC | |
| PBS 13 RT | AGTCGCCATGatatggggaagtgaatcagtacaaccgccacagtacCGGGCGGCG | |
| 29 AttB 36 | GAGA | |
| atgRNA | | |
| B. cereus | GCGTATTGCCTGGAGGATGGgttttagagctagaaatagcaagttaaaataaggctagtc | 350 |
| NOLC1_ | cgttatcaacttgaaaaagtggcaccgagtcggtgcGAACCACGCGGCGAATGCCGG | |
| PBS 13 RT | CGTCCGCCatatggggaagtgaatcagtacaaccgccacagtacTCCTCCAGGCAAT | |
| 29 AttB 36 | | |
| atgRNA | | |
| B. cereus | GGGCACGGGGCCATGTACAAgttttagagctagaaatagcaagttaaaataaggctagt | 351 |
| NOLC1_ | ccgttatcaacttgaaaaagtggcaccgagtcggtgcGGCGAATGCCGGCGTCCGC | |
| PBS 13 RT | CatatggggaagtgaatcagtacaaccgccacagtacTCCTCCAGGCAAT | |
| 20 AttB 36 | | |
| atgRNA | | |
| B. cereus | GGGCACGGGGCCATGTACAAgttttagagctagaaatagcaagttaaaataaggctagt | 352 |
| NOLC1_ | ccgttatcaacttgaaaaagtggcaccgagtcggtgcGGCGAATGCCGGCGTCCGC | |
| PBS 18 RT | CatatggggaagtgaatcagtacaaccgccacagtacTCCTCCAGGCAATACGCG | |
| 20 AttB 36 | | |
| atgRNA | | |
| B. cereus | GGGCACGGGGCCATGTACAAgttttagagctagaaatagcaagttaaaataaggctagt | 353 |
| SRRM2_ | ccgttatcaacttgaaaaagtggcaccgagtcggtgcGGCGTCGGCAGCCCGATCC | |
| PBS 9 RT 24 | CGTTGatatggggaagtgaatcagtacaaccgccacagtacTACATGGCC | |
| AttB 36 | | |
| atgRNA | | |
| B. cereus | GGGCACGGGGCCATGTACAAgttttagagctagaaatagcaagttaaaataaggctagt | 354 |
| SRRM2_ | ccgttatcaacttgaaaaagtggcaccgagtcggtgcGATCCCGTTGatatggggaagtgaat | |
| PBS 9 RT 10 | cagtacaaccgccacagtacTACATGGCC | |
| AttB 36 | | |
| atgRNA | | |
| B. cereus | GGGCACGGGGCCATGTACAAgttttagagctagaaatagcaagttaaaataaggctagt | 355 |
| SRRM2_ | ccgttatcaacttgaaaaagtggcaccgagtcggtgcGATCCCGTTGatatggggaagtgaat | |
| PBS 13 RT | cagtacaaccgccacagtacTACATGGCCCCGT | |
| 10 AttB 36 | | |
| atgRNA | | |
| Screen | GCTATTCTCGCAGCTCACCAgttttagagctagaaatagcaagttaaaataaggctagtc | 356 |
| validation | cgttatcaacttgaaaaagtggcaccgagtcggtgcgcgcggcgatatcatcatccatggatgatcctgac | |
| guides | gacggagaccgccgtcgtcgacaagcctgagctgcgag | |
| ACTB_1_11_ | | |
| 24_38 | | |
| Screen | GCTATTCTCGCAGCTCACCAgttttagagctagaaatagcaagttaaaataaggctagtc | 357 |
| validation | cgttatcaacttgaaaaagtggcaccgagtcggtgccgatatcatcatccatggoggatgatcctgacgac | |
| guides | ggagaccgccgtcgtcgacaagccggctgagctgcgagaatag | |
| ACTB_1_16_ | | |
| 18_43 | | |
| Screen | GCTGTCTCCGCCGCCCGCCAgttttagagctagaaatagcaagttaaaataaggctagtc | 358 |
| validation | cgttatcaacttgaaaaagtggcaccgagtcggtgcgcggcacgggggtcgcagtcgccatgatgatcct | |
| guides | gacgacggagaccgccgtcgtcgacaagcccgggcggc | |
| LMNB1_18_ | | |
| 26_38 | | |
| Screen | GCGTATTGCCTGGAGGATGGgttttagagctagaaatagcaagttaaaataaggctagtc | 359 |
| validation | cgttatcaacttgaaaaagtggcaccgagtcggtgcaatgccggcgtccgcccggatgatcctgacgacg | |
| guides | gagaccgccgtcgtcgacaagccggctcctccaggcaatac | |
| NOLC1_1_ | | |
| 15_16_43 | | |
| Screen | GCGTATTGCCTGGAGGATGGgttttagagctagaaatagcaagttaaaataaggctagtc | 360 |
| validation | cgttatcaacttgaaaaagtggcaccgagtcggtgcggcgtccgccatgatcctgacgacggagaccgcc | |
| guides | gtcgtcgacaagcctcctccaggcaata | |
| NOLC1 1 | | |
| 14 10 38 | | |
| Screen | GGGAAATGCATCTTGCACAAgttttagagctagaaatagcaagttaaaataaggctagtc | 361 |
| validation | cgttatcaacttgaaaaagtggcaccgagtcggtgcagcccctccatgctctctagctgttgccattgggctt | |
| guides | gtcgacgacggcggtctccgtcgtcaggatcattgcaagatgcatt | |
| SERPIN_13_ | | |
| 32_38 | | |
| Screen | GTGTCAGGTGGGGCGGGGCTAgttttagagctagaaatagcaagttaaaataaggctag | 362 |
| validation | tccgttatcaacttgaaaaagtggcaccgagtcggtgctggcaccataatgatcctgacgacggagaccgc | |
| guides | cgtcgtcgacaagccccccgccc | |
| DEPDC4_8_ | | |
| 10_38 | | |
| SERPIN | GTGGGGACAGCCCCGTCTCTgttttagagctagaaatagcaagttaaaataaggctagtc | 363 |
| Nicking | cgttatcaacttgaaaaagtggcaccgagtcggtgc | |
| guide −107 | | |
| guide | | |
| SERPIN | GCTCTTGGGAAAAAAACCCTAgttttagagctagaaatagcaagttaaaataaggctag | 364 |
| Nicking | tccgttatcaacttgaaaaagtggcaccgagtcggtgc | |
| guide −91 | | |
| guide | | |
| SERPIN | GTCTTGGGAAAAAAACCCTAAgttttagagctagaaatagcaagttaaaataaggctag | 365 |
| Nicking | tccgttatcaacttgaaaaagtggcaccgagtcggtgc | |
| guide −90 | | |
| guide | | |
| SERPIN | GAAAAAAACCCTAAGGGCTGgttttagagctagaaatagcaagttaaaataaggctagt | 366 |
| Nicking | ccgttatcaacttgaaaaagtggcaccgagtcggtgc | |
| guide −84 | | |
| guide | | |
| SERPIN | GCTGAGGATCCTTGTGAGTGTgttttagagctagaaatagcaagttaaaataaggctagt | 367 |
| Nicking | ccgttatcaacttgaaaaagtggcaccgagtcggtgc | |
| guide −67 | | |
| guide | | |
| SERPIN | GTGAGGATCCTTGTGAGTGTTgttttagagctagaaatagcaagttaaaataaggctagt | 368 |
| Nicking | ccgttatcaacttgaaaaagtggcaccgagtcggtgc | |
| guide −66 | | |
| guide | | |
| SERPIN | GGATCCTTGTGAGTGTTGGGgttttagagctagaaatagcaagttaaaataaggctagtc | 369 |
| Nicking | cgttatcaacttgaaaaagtggcaccgagtcggtgc | |
| guide −63 | | |
| guide | | |
| SERPIN | GATCCTTGTGAGTGTTGGGTgttttagagctagaaatagcaagttaaaataaggctagtc | 370 |
| Nicking | cgttatcaacttgaaaaagtggcaccgagtcggtgc | |
| guide −62 | | |
| guide | | |
| SERPIN | GTTGGGTGGGAACAGCTCCCgttttagagctagaaatagcaagttaaaataaggctagtc | 371 |
| Nicking | cgttatcaacttgaaaaagtggcaccgagtcggtgc | |
| guide −49 | | |
| guide | | |
| SERPIN | GGGTGGGAACAGCTCCCAGGgttttagagctagaaatagcaagttaaaataaggctagt | 372 |
| Nicking | ccgttatcaacttgaaaaagtggcaccgagtcggtgc | |
| guide −46 | | |
| guide | | |
| SERPIN | GCTTCTGTGCAGCAGTTTCCCgttttagagctagaaatagcaagttaaaataaggctagt | 373 |
| Nicking | ccgttatc aacttgaaaaagtggcaccgagtcggtgc | |
| guide +34 | | |
| guide | | |
| SERPIN | GTTTCCCTGGCCACTAAATAGgttttagagctagaaatagcaagttaaaataaggctagt | 374 |
| Nicking | ccgttatc aacttgaaaaagtggcaccgagtcggtgc | |
| guide +48 | | |
| guide | | |
| SERPIN | GTTCCCTGGCCACTAAATAGTgttttagagctagaaatagcaagttaaaataaggctagt | 375 |
| Nicking | ccgttatc aacttgaaaaagtggcaccgagtcggtgc | |
| guide +49 | | |
| guide | | |
| SERPIN | GATTAGATAGAAGCCCTCCAgttttagagctagaaatagcaagttaaaataaggctagtc | 376 |
| Nicking | cgttatca acttgaaaaagtggcaccgagtcggtgc | |
| guide +71 | | |
| guide | | |
| SERPIN | GATTAGATAGAAGCCCTCCAAgttttagagctagaaatagcaagttaaaataaggctag | 377 |
| Nicking | tccgttat caacttgaaaaagtggcaccgagtcggtgc | |
| guide +72 | | |
| guide | | |

### 6.8. Integrases/Recombinases and Integration/Recombination Sites

In typical embodiments, the single nucleic acid construct (i.e., “installer”) contains an integrase or recombinase. In some embodiments, the single nucleic acid construct (i.e., “installer”) contains an integrase and a recombinase. In some embodiments, the single nucleic acid construct (i.e., “installer”) contains at least one integrase (e.g., at least two integrases) and at least one recombinase (e.g., at least two recombinases). In some embodiments, an integration enzyme (e.g., an integrase or a recombinase) is selected from the group consisting of Cre, Dre, Vika, Bxb1, φC31, RDF, FLP, φBTl, R1, R2, R3, R4, R5, TP901-1, A118, φFCI, φC1, MR11, TG1, φ370.1, WB, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by a Tc1/mariner family member including but not limited to retrotransposases encoded by LI, Tol2, Tel, Tc3, Himar 1 (isolated from the horn fly, Haematobia irritans), Mos1 (Mosaic element of Drosophila mauritiana), and Minos, and any mutants thereof. As can be used herein, Xu et al describes methods for evaluating integrase activity in E. coli and mammalian cells and confirmed at least R4, φC31, φBT1, Bxb1, SPBc, TP901-1 and WB integrases to be active on substrates integrated into the genome of HT1080 cells (Xu et al., 2013, Accuracy and efficiency define Bxb1 integrase as the best of fifteen candidate serine recombinases for the integration of DNA into the human genome. BMC Biotechnol. 2013 Oct. 20; 13:87. doi: 10.1186/1472-6750-13-87). Durrant describes new large serine recombinases (LSRs) divided into three classes distinguished from one another by efficiency and specificity, including landing pad LSRs which outperform wild-type Bxb1 in episomal and chromosomal integration efficiency, LSRs that achieve both efficient and site-specific integration without a landing pad, and multi-targeting LSRs with minimal site-specificity. Additionally, embodiments can include any serine recombinase such as BceINT, SSCINT, SACINT, and INT10 (see Ionnidi et al., 2021; Drag- and-drop genome insertion without DNA cleavage with CRISPR directed integrases. bioRxty 2021.11.01 466786, doi.org/10.1101/2021.11.01.466786). In some embodiments, the integration site can be selected from an attB site, an attP site, an attL site, an attR site, a lox71 site a Vox site, or a FRT site.

In one embodiment, the single nucleic acid construct (i.e., “installer”) contains an integrase (e.g., any of the integreases described herein (e.g., any of the large serine integrases described herein). In one embodiment, the single nucleic acid construct (i.e., “installer”) contains a recombinase (e.g., any of the recombinases described herein). In some embodiments, the single nucleic acid construct (i.e., “installer”) contains a large serine integrase (e.g., any of the large serine integrases described herein) and a recombinase. In some embodiments, the single nucleic acid construct (i.e., “installer”) contains a B×B1 integrase and a flippase (e.g., FLP).

It will be appreciated that desired activity of integrases, transposases and the like can depend on nuclear localization. In certain embodiments, prokaryotic enzymes are adapted to modulate nuclear localization. In certain embodiments, eukaryotic or vertebrate enzymes are adapted to modulate nuclear localization. In certain embodiments, the invention provides fusion or hybrid proteins. Such modulation can comprise addition or removal of one or more nuclear localization signal (NLS) and/or addition or removal of one or more nuclear export signal (NES). Xu et al compared derivatives of fourteen serine integrases that either possess or lack a nuclear localization signal (NLS) to conclude that certain integrases benefit from addition of an NLS whereas others are transported efficiently without addition, and a major determinant of activity in yeast and vertebrate cells is avoidance of toxicity. (Xu et al., 2016, Comparison and optimization of ten phage encoded serine integrases for genome engineering in Saccharomyces cerevisiae. BMC Biotechnol. 2016 Feb. 9; 16:13. doi: 10.1186/s12896-016-0241-5). Ramakrishnan et al. systematically studied the effect of different NES mutants developed from mariner-like elements (MLEs) on transposase localization and activity and concluded that nuclear export provides a means of controlling transposition activity and maintaining genome integrity. (Ramakrishnan et al. Nuclear export signal (NES) of transposases affects the transposition activity of mariner-like elements Ppmar 1 and Ppmar 2 of moso bamboo. Mob DNA. 2019 Aug. 19; 10:35. doi: 10.1186/s13100-019-0179-y). The methods and constructs are used to modulate nuclear localization of system components of the invention.

In typical embodiments, the integrase used herein is selected from below.

TABLE 10
Integrases

| | | | | protein | | | | | | | | | |
|---|---|---|---|---|---|---|---|---|---|---|---|---|---|
| | | | nucleo- | acc- | | | | | | | | | |
| | SRA | bio | tide | ession | internal | | Alter- | | | | SEQ | | |
| Data | acc- | project_ | acc- | or ORF | protein | Proposed | native | organism/ | des- | | ID | | |
| base | ession | acc | ession | ID | ID | names | names | source | cription | Sequence | NO: | Length | Group |
| ENA | SRS | PRJ | NA | NA | N189929_ | SsuINT | NA | human | stool | MEKNRAVLYLRLSKEDVDKVN | 378 | 527 | INTc |
| | 1205 | EB2 | | | 49_54 | | | gutmetage | sample | KGDDSSSIKSQRLLLTDFALERG | | | |
| | 298 | 6277 | | | | | | nome | from | FKIVGVYSDDDESGLYDDRPDF | | | |
| | | | | | | | | | male in | ERMMTDAKLDEFDIIIAKTQSRF | | | |
| | | | | | | | | | USA | SRNMEHIEKYLHHDLPNLGIRFI | | | |
| | | | | | | | | | | GAVDGVDTESDENKKSRQINGL | | | |
| | | | | | | | | | | VNEWYCEDLSKNIRSAFKAKM | | | |
| | | | | | | | | | | KDGQFLGSSCPYGYKKDPQNH | | | |
| | | | | | | | | | | NHLVVDDYAAKVVQKIFNLYL | | | |
| | | | | | | | | | | EGYGKAKIGSILSSEGILIPTLYK | | | |
| | | | | | | | | | | KDILKQNYHNSKALDTTQNWS | | | |
| | | | | | | | | | | YQTIHTILNNEVYLGHLIQNKV | | | |
| | | | | | | | | | | NTMSYKDKNKRILPKEKWIIVR | | | |
| | | | | | | | | | | NTHEPIITEEMFQDVQKLQKNR | | | |
| | | | | | | | | | | TRSVENIEPNGLFSGLIFCADCK | | | |
| | | | | | | | | | | HAMSRKYARRGEKGFVGYVCK | | | |
| | | | | | | | | | | TYKTQGKNFCESHSIDYDELEE | | | |
| | | | | | | | | | | AVLFSIKNEARSILQQEEIDELR | | | |
| | | | | | | | | | | KVQAYDETKSYYEMQLENIKSR | | | |
| | | | | | | | | | | MEKIEKYKKKTYDNYMDDLIS | | | |
| | | | | | | | | | | RDDYKKYVTEYDKEIGGLKQQ | | | |
| | | | | | | | | | | QELINSKTDLEKEISTQYDEWVE | | | |
| | | | | | | | | | | AFINYVDIDKLTREIVIELIEKIEV | | | |
| | | | | | | | | | | NKDGSINIYYKFKNPYIS | | | |
| ENA | ERS | PRJ | NA | NA | N190156_ | SssINT | NA | human | stool | MNTVIYARYSAGPRQTDQSIDG | 379 | 510 | INTd |
| | 3964 | EB2 | | | 234_12 | | | gut | sample | QLRVCTEFCKQRGLTVVDTYC | | | |
| | 61 | 6280 | | | | | | metagenome | from | DRHISGRTDERPEFQRLIADAKA | | | |
| | | | | | | | | | Spain | HKFEAVVVYKTDRFARNKYDS | | | |
| | | | | | | | | | | AIYKRELRRNGIQIFYAAEAIPE | | | |
| | | | | | | | | | | GPEGIILESLMEGLAEYYSAELA | | | |
| | | | | | | | | | | QKIKRGLNESALKCQSLGSGRP | | | |
| | | | | | | | | | | LGYTVDEQKHFQIDPESSQAVK | | | |
| | | | | | | | | | | TIFEMYIKGESNAAICDYLNARG | | | |
| | | | | | | | | | | LRTSQGNLFNKNSINRIIKNRKY | | | |
| | | | | | | | | | | IGEYRYNDIVVEGGMPAIISKET | | | |
| | | | | | | | | | | FCMAQAEMERRRTHRAPVSPK | | | |
| | | | | | | | | | | AEYLLAGKLFCGHCKGPMQGV | | | |
| | | | | | | | | | | SGTGKSGNKWYYYYCANTRGK | | | |
| | | | | | | | | | | ERTCDKKQVSRDRLEKAVVDF | | | |
| | | | | | | | | | | TVRYILQENVLEELSKKVYAAQ | | | |
| | | | | | | | | | | ERQNNTASEIAFYEKKLAENKK | | | |
| | | | | | | | | | | AIANILRAIESGAMTQALPARLQ | | | |
| | | | | | | | | | | ELENEQTVIQGELSYLKGARLA | | | |
| | | | | | | | | | | FTEDQILFALLQHLDPRPGESER | | | |
| | | | | | | | | | | DYHRRIITDFVSEVYLYDDRMLI | | | |
| | | | | | | | | | | YFNISSADGKLKHADLSAIESGV | | | |
| | | | | | | | | | | FDAGLISSSSRASSFSTRCALI | | | |
| ENA | ERS | PRJ | NA | NA | N191352_ | SscINT | NA | human | stool | MNEKNLEIGAAYIRVSTDDQTE | 380 | 482 | INTd |
| | 1015 | EB2 | | | 143_72 | | | gutmetage | sample | LSPDAQLRVILEAAKKDGIIIPQE | | | |
| | 837 | 6832 | | | | | | nome | from | FVFMEDRGRSGRRADNRPEFQR | | | |
| | | | | | | | | | China | MISTARQNPSPFRYLYLWKFSR | | | |
| | | | | | | | | | | FARNQEESAFYKGILRKKCGVTI | | | |
| | | | | | | | | | | KSVSEPIMEGMFGRLVEMIIEWS | | | |
| | | | | | | | | | | DEFYSVNLSGEVLRGMTQKALE | | | |
| | | | | | | | | | | HGYQLTPCLGYDAVGHGRPYVI | | | |
| | | | | | | | | | | NEEQYQIVEFIHRSFFDGKDMT | | | |
| | | | | | | | | | | WIAREANRRGYHTRRGNPFDTR | | | |
| | | | | | | | | | | AVRIILTNSFYVGLVKWNDVTF | | | |
| | | | | | | | | | | QGTHECRESVTSVFSANQERLN | | | |
| | | | | | | | | | | RIHRPRGRRQASSCKHWLSGLL | | | |
| | | | | | | | | | | KCSICGASLGYNQTKDLTKRGH | | | |
| | | | | | | | | | | AFQCWKYTKGIHPGSCSVSSLK | | | |
| | | | | | | | | | | AEAAVLESLQMILETGEVEYTY | | | |
| | | | | | | | | | | EQREKHLDDNKLTLIQKSLERL | | | |
| | | | | | | | | | | DTKELRIREAYESGIDTLDEFKT | | | |
| | | | | | | | | | | NKARLQRERDQLMEELEELHSQ | | | |
| | | | | | | | | | | EEPEDVPGKEILIERIQNVYDLL | | | |
| | | | | | | | | | | QSPDVDNDDKGNAVRSIIKKIV | | | |
| | | | | | | | | | | YIKESKTFCFYYYV | | | |
| ENA | ERS | PRJ | NA | NA | N191533_ | Ssc2INT | NA | human | stool | MERTIKVIQPGTVKIPTKKRVAA | 381 | 406 | INTc |
| | 1289 | EB2 | | | 224_76 | | | gutmetage | sample | YARVSSGKDAMLHSLSAQVSY | | | |
| | 677 | 6924 | | | | | | nome | from | YSNMIQQKNEWSYVGIYADEAI | | | |
| | | | | | | | | | China | TGTKDRRVEFNRLIQDCTDGKI | | | |
| | | | | | | | | | | DMIITKSISRFARNTLTMLEVVR | | | |
| | | | | | | | | | | KLKNINVDVYFEKENIHSISGDG | | | |
| | | | | | | | | | | ELMLTILASFAQEESRSVSENCK | | | |
| | | | | | | | | | | WRIRKGFEQGELINLRFLYGYRI | | | |
| | | | | | | | | | | NKGKIEIYEKEAEIVRMIFDDYL | | | |
| | | | | | | | | | | NGEGCTRIGNKLRKMKVNKLR | | | |
| | | | | | | | | | | GGMWNSERVVDIIKNEKYTGN | | | |
| | | | | | | | | | | ALLQKKYVKDHLSKKLVRNKG | | | |
| | | | | | | | | | | ILTQYYAEGTHPAIIDIKTFEIAQ | | | |
| | | | | | | | | | | KIMEANRTKFQGKCGSNRYLFT | | | |
| | | | | | | | | | | SKIECGICGKNYRHKDREGKST | | | |
| | | | | | | | | | | WVCANHLKYGNSRCIAKPLNE | | | |
| | | | | | | | | | | EKLKKLINEALELKYFDEEIFIR | | | |
| | | | | | | | | | | NIKRIKVTGNQTIEFILKDGKVIE | | | |
| | | | | | | | | | | EGMI | | | |
| ENA | ERS | PRJ | NA | NA | N203911_ | SsdINT | NA | human | stool | MKKIKIDRAIQERPATRKQTRN | 382 | 401 | INTc |
| | 265 | EB2 | | | 45186_6 | | | gut | sample | EKIRQSLTEHVDVQVIPAITDRE | | | |
| | 5827 | 8245 | | | | | | metagenome | from | GYEKPKLRVCAYCRVSTDMDT | | | |
| | | | | | | | | | Denmark | QALSYELQVQNYTDYIRGNDE | | | |
| | | | | | | | | | | WRFAGIYADRGISGTSLKHRDE | | | |
| | | | | | | | | | | FNRMIEDCKAGKIDLIITKAVTR | | | |
| | | | | | | | | | | FARNVLDCISTIRMLKQLEHPV | | | |
| | | | | | | | | | | AVYFETERINTLDTTSETYLGLI | | | |
| | | | | | | | | | | SLFAQGESESKSESLKWSYIRR | | | |
| | | | | | | | | | | WKRGTGIYPAWSLLGYEMGED | | | |
| | | | | | | | | | | GKWQIVEAEAELVRIIYDMYLN | | | |
| | | | | | | | | | | GYSSPQIAEILTRSGVPTATNQT | | | |
| | | | | | | | | | | VWSSGGVLGILRNEKYCGNVL | | | |
| | | | | | | | | | | CQKTMTVDVFSHKAIKNTGQK | | | |
| | | | | | | | | | | TQYFIEGHHDPIILRSDWDRVQQ | | | |
| | | | | | | | | | | MIDEKYYRKRRGRRTKPRIVLK | | | |
| | | | | | | | | | | GCLAGFTQIDLDWDEDDIARIF | | | |
| | | | | | | | | | | YSTTPAAEVATPAMADHIEIIKV | | | |
| | | | | | | | | | | KGEN | | | |
| ENA | SRS | PRJ | NA | NA | N208621_ | SmcINT | NA | human | sample | MKTAAAYIRVSTDDQVEYSPDS | 383 | 476 | INTd |
| | 2949 | EB3 | | | 9_15 | | | gut | from 72- | QIKLIRDYAKRNDYILPDEFIFR | | | |
| | 42 | 0046 | | | | | | metagenome | year-old | DDGISGKSAKHRPEFTKMIALA | | | |
| | | | | | | | | | male | KSPEHPFDAILVWKFSRFARNQ | | | |
| | | | | | | | | | from | EESIVFKNILRKIGVEVRSVSEPI | | | |
| | | | | | | | | | China | SEDPFGSLVERIIEWTDEYYIINL | | | |
| | | | | | | | | | | SGEVKRGMLEKISRGQPVVPPP | | | |
| | | | | | | | | | | VGYKMENGQYIPDENAHFIKEI | | | |
| | | | | | | | | | | FEAYAAGEGARHIAQRLAAQG | | | |
| | | | | | | | | | | CLTKRGNPIDNRFVDYVLHNPV | | | |
| | | | | | | | | | | YIGKLRWSVNSHAASSRHYDSA | | | |
| | | | | | | | | | | DIIVFDGTHEPLISSELWESVQK | | | |
| | | | | | | | | | | RLHEVKTLYPKYQRREQPVSFM | | | |
| | | | | | | | | | | LKGLVRCSSCGSTLCYCRTSEPS | | | |
| | | | | | | | | | | LQCHSYARGSCRQSHSINIATAN | | | |
| | | | | | | | | | | EAVIKGLQLAVDKLDFAIAPAK | | | |
| | | | | | | | | | | PHYSADAPGTNKLLAAEYKKM | | | |
| | | | | | | | | | | ERIKAAYANGTDTLEEYAANK | | | |
| | | | | | | | | | | KKISAEIARLEAELQQESNVKPI | | | |
| | | | | | | | | | | NKKAFAKRVSEIIKYISDPHNSE | | | |
| | | | | | | | | | | AAKNQALRTVISYIIFDRAATTF | | | |
| | | | | | | | | | | NIIFHF | | | |
| Met | NA | NA | NA | NA | N675015_ | UhmINT | NA | urban | NA | MKIAIYARKSKYSPTGESVENQI | 384 | 550 | INTd |
| aSUB | | | | | 95_5 | | | human | | QLCKEYLQAKYKSETLEIDEYK | | | |
| | | | | | | | | microbiome | | DEGYSGGNTNRPDFKKLIAQIE | | | |
| | | | | | | | | | | DYDMLICYRLDRISRNVADFSS | | | |
| | | | | | | | | | | TLTLLQNNKCDFVSIKEQFDTTS | | | |
| | | | | | | | | | | PMGRAMIYISSVFAQLERETIAE | | | |
| | | | | | | | | | | RIRDNMMELAKMGRWLGGTIP | | | |
| | | | | | | | | | | MGFDSEPITFIDENMKERSMTK | | | |
| | | | | | | | | | | LIPNVEELKVIELIYEKYLQLGS | | | |
| | | | | | | | | | | MGKVVTYLLQNNIKTKKGKDF | | | |
| | | | | | | | | | | TLGSIKVILTNPIYVKANQEVVN | | | |
| | | | | | | | | | | HLKTQGITICGDVDGKKALLTY | | | |
| | | | | | | | | | | NKTTGISNDVGTKTIVKDKSEW | | | |
| | | | | | | | | | | IAAVANHKGIIPADKWLQAQNI | | | |
| | | | | | | | | | | KDKNKDSFPALGRSNTTIASRV | | | |
| | | | | | | | | | | LRCDKCESTMGVTHGHINPVTG | | | |
| | | | | | | | | | | KKHYYYNCTLKKRSKGVRCDN | | | |
| | | | | | | | | | | KPAKAAEVDEAILITLENMFKA | | | |
| | | | | | | | | | | KSSIIDNLKAKNKARRIEMISSN | | | |
| | | | | | | | | | | RVDVINKIIEDKTKQIDNL VNKL | | | |
| | | | | | | | | | | SLDDDLTDILFKKIKGLKAEIKE | | | |
| | | | | | | | | | | LEDELLTLTSDNIKLNEDEVVLD | | | |
| | | | | | | | | | | FTEKLLEKCSIIRTLDILEQQQIV | | | |
| | | | | | | | | | | DALIPLVTWNGDTEVLNIYPLG | | | |
| | | | | | | | | | | SPELELKEAESKKK | | | |
| Sega | NA | PRJ | NA | NA | N684346_ | SacINT | NA | human | stool | MKEKVSERKTGAIYIRVSTDKQ | 385 | 493 | INTd |
| ta- | | NA4 | | | 90_69 | | | gut | sample | EELSPDAQLRLLLDYAKKDSID | | | |
| Paso | | 2243 | | | | | | metagenome | from | VPKEYIFQDNGISGRKANKRPA | | | |
| lli | | 4 | | | | | | | adult in | FQNMIALAKSKEHPIDTIIVWKF | | | |
| | | | | | | | | | China | SRFARNQEESIVYKSLLKKNNV | | | |
| | | | | | | | | | | DVVSVSEPLIDGPFGSLIERIIEW | | | |
| | | | | | | | | | | MDEYYSIRLSGEVMRGMTQNA | | | |
| | | | | | | | | | | MRGHYQSDAPIGYTSPGDKKPP | | | |
| | | | | | | | | | | VINPDTVQIPLMIKDMFLSGSTQ | | | |
| | | | | | | | | | | LQIARKLNDSGYRTKRGNLWD | | | |
| | | | | | | | | | | ARGVRYVLENPFYIGKSRWNYT | | | |
| | | | | | | | | | | ERGRRLKPADEVIYADGNWEA | | | |
| | | | | | | | | | | LWDEDTFKEIQKRLALNMRKS | | | |
| | | | | | | | | | | KSRDISAAKHWLSGLLICSSCGG | | | |
| | | | | | | | | | | TLAFGGAHNMRGFQCWKYSKG | | | |
| | | | | | | | | | | FCSESHYISTGPIEKMVLEYLEA | | | |
| | | | | | | | | | | VMHSPALSYTVISSSSVDASSKL | | | |
| | | | | | | | | | | SDLERQLQKIDAKEKRIKAAYL | | | |
| | | | | | | | | | | NEIDTLEEYKANKTALEEERRT | | | |
| | | | | | | | | | | VEKEIEELTLSDVKYSKEDLDK | | | |
| | | | | | | | | | | KMKQNISDLLRVLRDESADYIQ | | | |
| | | | | | | | | | | KGNMMRNVVDHIVFNRKNTSL | | | |
| | | | | | | | | | | DVFLKLVV | | | |
| Sega | ERR | PRJ | NA | NA | N687611_ | RsaINT | NA | human | rectal | MKITKKQPLRPRGRSEDKRQST | 386 | 404 | INTc |
| ta- | 1136 | EB1 | | | 90_68 | | | gut | swab | KNVIRDAYINGPQKEVQIIPAKR | | | |
| Paso | 864 | 1532 | | | | | | metagenome | from | DMEAETEKKKLRVCAYCRVST | | | |
| Li | | | | | | | | | adult in | DEDTQASSYELQVQNYTRMIRE | | | |
| | | | | | | | | | Isreal | NPEWEFAGIFADEGISGTSVLHR | | | |
| | | | | | | | | | | EHFLEMIEKCKAGEIDLIITKQV | | | |
| | | | | | | | | | | SRFARNVLDSLNYIFMLRKLDP | | | |
| | | | | | | | | | | PVGVYFETEKLNTLDKSSDMVI | | | |
| | | | | | | | | | | TVLSLVAQSESEQKSNSLKWSF | | | |
| | | | | | | | | | | KRRRAQGLGIYPSWALLGYRLD | | | |
| | | | | | | | | | | DEKNWEIVEDEADIVRTIYSLYL | | | |
| | | | | | | | | | | DGYSSTQIAELLTKSGIPTVKGL | | | |
| | | | | | | | | | | SVWSSGSVLGILKNEKFCGDAL | | | |
| | | | | | | | | | | CQKTVTIDFFTHKSVKNNGIEPQ | | | |
| | | | | | | | | | | YFVEGHHIPIIEKNDWLLAQQIR | | | |
| | | | | | | | | | | KERRYRKRRSTHRKPRIVVKGA | | | |
| | | | | | | | | | | LSGFMIVDTSWDEEYVDSLLISA | | | |
| | | | | | | | | | | TQKPEPAPVIAEEDENFIVIEKE | | | |
| Sega | ERR | PRJ | NA | NA | N687663_ | Rsa2INT | NA | human | rectal | MADIQPVKNGALYIRVSTHLQE | 387 | 498 | INTd |
| ta- | 1136 | EB1 | | | 53_29 | | | gut | swab | ELSPDAQKRLLMEYAEAHNIIV | | | |
| Paso | 737 | 1532 | | | | | | metagenome | from | LKEHIYIDSGISGRSARQRPQFN | | | |
| lli | | | | | | | | | adult in | NMIAEAKSKEHPFDVILVWKYS | | | |
| | | | | | | | | | Isreal | RFARNQEESIVYKSMLKRENVD | | | |
| | | | | | | | | | | VISVSEPISDDPFGSLIERIIEWM | | | |
| | | | | | | | | | | DEYYSIRLSGEVSRGMAENAMR | | | |
| | | | | | | | | | | GNYQARPPLGYRIPGYRQTPVI | | | |
| | | | | | | | | | | VPEEAELIQLIFDLYTEKKMGIF | | | |
| | | | | | | | | | | EIVRYLNEHGYQTGHKKPFQRR | | | |
| | | | | | | | | | | SVTYILKNPTYIGKTIWNQHDQ | | | |
| | | | | | | | | | | DHKLRDKSEWIIADGKHEPIISK | | | |
| | | | | | | | | | | EQFDKAQKRIESTYKPAYRKPT | | | |
| | | | | | | | | | | SVCHHWLSSLLKCSSCGRTLVV | | | |
| | | | | | | | | | | KRTASKKKDRMYVNFQCYGYQ | | | |
| | | | | | | | | | | KGICNTNQSISAIKLEPVIMHAL | | | |
| | | | | | | | | | | EDAMTSGKIHFDVLNPTTLDSS | | | |
| | | | | | | | | | | QKQQFLTRLNEIEKKEERIKRAY | | | |
| | | | | | | | | | | RDGIDTLEEYKENKSIIQTEKEM | | | |
| | | | | | | | | | | LLKKIEHIEEPALSPEEAKPIMM | | | |
| | | | | | | | | | | DRIKNVYEIITNPDIGMEEKNKA | | | |
| | | | | | | | | | | ARSIIEKIVFDRATGSVNIFFYLA | | | |
| | | | | | | | | | | HCP | | | |
| NCBI | NA | NA | NC_ | NP_ | NA | BxbINT | Bxb1 | Myco- | NA | MRALVVIRLSRVTDATTSPERQ | 388 | 501 | INTa |
| | | | 0026 | 07530 | | | inte- | bacterium | | LESCQQLCAQRGWDVVGVAED | | | |
| | | | 56.1 | 2.1 | | | grase | phage | | LDVSGAVDPFDRKRRPNLARW | | | |
| | | | | | | | | Bxb1 | | LAFEEQPFDVIVAYRVDRLTRSI | | | |
| | | | | | | | | | | RHLQQLVHWAEDHKKLVVSAT | | | |
| | | | | | | | | | | EAHFDTTTPFAAVVIALMGTVA | | | |
| | | | | | | | | | | QMELEAIKERNRSAAHFNIRAG | | | |
| | | | | | | | | | | KYRGSLPPWGYLPTRVDGEWR | | | |
| | | | | | | | | | | LVPDPVQRERILEVYHRVVDNH | | | |
| | | | | | | | | | | EPLHLVAHDLNRRGVLSPKDYF | | | |
| | | | | | | | | | | AQLQGREPQGREWSATALKRS | | | |
| | | | | | | | | | | MISEAMLGYATLNGKTVRDDD | | | |
| | | | | | | | | | | GAPLVRAEPILTREQLEALRAEL | | | |
| | | | | | | | | | | VKTSRAKPAVSTPSLLLRVLFC | | | |
| | | | | | | | | | | AVCGEPAYKFAGGGRKHPRYR | | | |
| | | | | | | | | | | CRSMGFPKHCGNGTVAMAEW | | | |
| | | | | | | | | | | DAFCEEQVLDLLGDAERLEKV | | | |
| | | | | | | | | | | WVAGSDSAVELAEVNAELVDL | | | |
| | | | | | | | | | | TSLIGSPAYRAGSPQREALDARI | | | |
| | | | | | | | | | | AALAARQEELEGLEARPSGWE | | | |
| | | | | | | | | | | WRETGQRFGDWWREQDTAAK | | | |
| | | | | | | | | | | NTWLRSMNVRLTFDVRGGLTR | | | |
| | | | | | | | | | | TIDFGDLQEYEQHLRLGSVVER | | | |
| | | | | | | | | | | LHTGMS* | | | |
| NCBI | NA | NA | NC _ | NP_ | NA | Tp9INT | TP901- | Lacto- | NA | MTKKVAIYTRVSTTNQAEEGFS | 389 | 486 | INTd |
| | | | 0027 | 11266 | | | linte- | coccus | | IDEQIDRLTKYAEAMGWQVSDT | | | |
| | | | 47.1 | 4.1 | | | grase | phage | | YTDAGFSGAKLERPAMQRLIND | | | |
| | | | | | | | | TP901-1 | | IENKAFDTVLVYKLDRLSRSVR | | | |
| | | | | | | | | | | DTLYLVKDVFTKNKIDFISLNES | | | |
| | | | | | | | | | | IDTSSAMGSLFLTILSAINEFERE | | | |
| | | | | | | | | | | NIKERMTMGKLGRAKSGKSMM | | | |
| | | | | | | | | | | WTKTAFGYYHNRKTGILEIVPL | | | |
| | | | | | | | | | | QATIVEQIFTDYLSGISLTKLRD | | | |
| | | | | | | | | | | KLNESGHIGKDIPWSYRTLRQT | | | |
| | | | | | | | | | | LDNPVYCGYIKFKDSLFEGMHK | | | |
| | | | | | | | | | | PIIPYETYLKVQKELEERQQQTY | | | |
| | | | | | | | | | | ERNNNPRPFQAKYMLSGMARC | | | |
| | | | | | | | | | | GYCGAPLKIVLGHKRKDGSRT | | | |
| | | | | | | | | | | MKYHCANRFPRKTKGITVYND | | | |
| | | | | | | | | | | NKKCDSGTYDLSNLENTVIDNL | | | |
| | | | | | | | | | | IGFQENNDSLLKIINGNNQPILDT | | | |
| | | | | | | | | | | SSFKKQISQIDKKIQKNSDLYLN | | | |
| | | | | | | | | | | DFITMDELKDRTDSLQAEKKLL | | | |
| | | | | | | | | | | KAKISENKFNDSTDVFELVKTQ | | | |
| | | | | | | | | | | LGSIPINELSYDNKKKIVNNLVS | | | |
| | | | | | | | | | | KVDVTADNVDIIFKFQLA* | | | |
| NCBI | NA | NA | NC_ | NP_ | NA | Bt1INT | PhiBT | Strepto- | NA | MSPFIAPDVPEHLLDTVRVFLY | 390 | 595 | INTa |
| | | | 004664. | 813744. | | | inte- | myces | | ARQSKGRSDGSDVSTEAQLAA | | | |
| | | | 2 | 2 | | | grase | virus | | GRALVASRNAQGGARWVVAG | | | |
| | | | | | | | | phiBT1 | | EFVDVGRSGWDPNVTRADFER | | | |
| | | | | | | | | | | MMGEVRAGEGDVVVVNELSRL | | | |
| | | | | | | | | | | TRKGAHDALEIDNELKKHGVRF | | | |
| | | | | | | | | | | MSVLEPFLDTSTPIGVAIFALIAA | | | |
| | | | | | | | | | | LAKQDSDLKAERLKGAKDEIAA | | | |
| | | | | | | | | | | LGGVHSSSAPFGMRAVRKKVD | | | |
| | | | | | | | | | | NLVISVLEPDEDNPDHVELVER | | | |
| | | | | | | | | | | MAKMSFEGVSDNAIATTFEKEK | | | |
| | | | | | | | | | | IPSPGMAERRATEKRLASIKARR | | | |
| | | | | | | | | | | LNGAEKPIMWRAQTVRWILNH | | | |
| | | | | | | | | | | PAIGGFAFERVKHGKAHINVIRR | | | |
| | | | | | | | | | | DPGGKPLTPHTGILSGSKWLEL | | | |
| | | | | | | | | | | QEKRSGKNLSDRKPGAEVEPTL | | | |
| | | | | | | | | | | LSGWRFLGCRICGGSMGQSQG | | | |
| | | | | | | | | | | GRKRNGDLAEGNYMCANPKG | | | |
| | | | | | | | | | | HGGLSVKRSELDEFVASKVWA | | | |
| | | | | | | | | | | RLRTADMEDEHDQAWIAAAAE | | | |
| | | | | | | | | | | RFALQHDLAGVADERREQQAH | | | |
| | | | | | | | | | | LDNVRRSIKDLQADRKAGLYV | | | |
| | | | | | | | | | | GREELETWRSTVLQYRSYEAEC | | | |
| | | | | | | | | | | TTRLAELDEKMNGSTRVPSEWF | | | |
| | | | | | | | | | | SGEDPTAEGGIWASWDVYERR | | | |
| | | | | | | | | | | EFLSFFLDSVMVDRGRHPETKK | | | |
| | | | | | | | | | | YIPLKDRVTLKWAELLKEEDEA | | | |
| | | | | | | | | | | SEATERELAAL* | | | |
| NCBI | NA | NA | NC_ | WP_ | NA | BceINT | NA | Bacillusce | NA | MYPYDVPDYAGSYRPESLDVCI | 391 | 529 | INTc |
| | | | 011658. | 0002 | | | | reus AH187 | | YLRKSRKDVEEERRAIEEGSSY | | | |
| | | | 1 | 86206. | | | | | | NALERHRKRLFAIAKAENHNIID | | | |
| | | | | 1 | | | | | | IFEEVASGESIQERPQMQQLLRK | | | |
| | | | | | | | | | | LEGNEIDGVLVIDLDRLGRGDM | | | |
| | | | | | | | | | | LDAGMIDRAFRYSSTKIITPTDV | | | |
| | | | | | | | | | | YDPDDESWELVFGIKSLISRQEL | | | |
| | | | | | | | | | | KSITKRLQNGRIDSVKEGKHIGK | | | |
| | | | | | | | | | | KPPYGYLKDENLRLYPDPEKA | | | |
| | | | | | | | | | | WIVKKIFELMCDGKGRQMIAAE | | | |
| | | | | | | | | | | LDRLGIDPPVTKRGAWDSSTITS | | | |
| | | | | | | | | | | IIKNEVYTGVIVWGKFKHKKRN | | | |
| | | | | | | | | | | GKYTRHKNPQEKWIMYENAHE | | | |
| | | | | | | | | | | PIISKELFDAANEAHSSRHKPAV | | | |
| | | | | | | | | | | ITSKKLTNPLAGILKCKLCG | | | |
| | | | | | | | | | | YTMLIQTRKDRPHNYLRCNNPA | | | |
| | | | | | | | | | | CKGKQKQSVFNLVEEKLLYSLQ | | | |
| | | | | | | | | | | QIVDEY | | | |
| | | | | | | | | | | QAQKVEEVEIDDSKLISFKEKAII | | | |
| | | | | | | | | | | SKE | | | |
| | | | | | | | | | | KELKELQAQKGNLHDLLEQGIY | | | |
| | | | | | | | | | | TVE | | | |
| | | | | | | | | | | IFLERQKNLVERITSIENDIEVLQ | | | |
| | | | | | | | | | | KEIE | | | |
| | | | | | | | | | | TEQIKEHNKTEFIPALKTVIESY | | | |
| | | | | | | | | | | HKTT | | | |
| | | | | | | | | | | NIELKNOLLKTILSTVTYYRHPD | | | |
| | | | | | | | | | | WKTNEFEIQVYFKIS* | | | |
| NCBI | NA | NA | NC_ | WP_ | NA | BcyINT | NA | Bacillus | NA | MYPYDVPDYAGSAVGIYIRVST | 392 | 487 | INTd |
| | | | 009674. | 0120954 | | | | cyto- | | QEQASEGHSIESQKKKLASYCEI | | | |
| | | | 1 | 29.1 | | | | toxicus | | QGWDDYRFYIEEGISGKNTNRP | | | |
| | | | | | | | | NVH391-98 | | KLKLLMEHIEKGKINILLVYRLD | | | |
| | | | | | | | | | | RLTRSVIDLHKLLNFLQEHGCA | | | |
| | | | | | | | | | | FKSATETYDTTTANGRMSMGIV | | | |
| | | | | | | | | | | SLLAQWETENMSERIKLNLEHK | | | |
| | | | | | | | | | | VLVEGERVGAIPYGFDLSDDEK | | | |
| | | | | | | | | | | LVKNEKSAILLDMVERVENGW | | | |
| | | | | | | | | | | SVNRIVNYLNLTNNDRNWSPN | | | |
| | | | | | | | | | | GVLRLLRNPALYGATRWNDKI | | | |
| | | | | | | | | | | AENTHEGIISKERFNRLQQILAD | | | |
| | | | | | | | | | | RSIHHRRDVKGTYIFQGVLRCP | | | |
| | | | | | | | | | | VCDQTLSVNRFIKKRKDGTEYC | | | |
| | | | | | | | | | | GVLYRCQPCIKQNKYNLAIGEA | | | |
| | | | | | | | | | | RFLKALNEYMSTVEFQTVEDEV | | | |
| | | | | | | | | | | IPKKSEREMLESQLQQIARKREK | | | |
| | | | | | | | | | | YQKAWASDLMSDDEFEKLMVE | | | |
| | | | | | | | | | | TRETYDECKQKLESCEDPIKIDE | | | |
| | | | | | | | | | | TYLKEIVYMFHQTFNDLESEKQ | | | |
| | | | | | | | | | | KEFISKFIRTIRYTVKEQQPIRPD | | | |
| | | | | | | | | | | KSKTGKGKQKVIITEVEFYQS* | | | |
| NCBI | NA | NA | NC_ | WP_ | NA | SluINT | NA | Staphy- | NA | MYPYDVPDYAGSKVAIYTRVSS | 393 | 473 | INTd |
| | | | 0173 | 3323 | | | | lococcus | | AEQANEGYSIHEQKKKLISYCEI | | | |
| | | | 53.1 | 0145 | | | | lugd- | | HDWNEYKVFTDAGISGGSMKR | | | |
| | | | | 8.1 | | | | unensis | | PALQKLMKHLSSFDLVLVYKLD | | | |
| | | | | | | | | N920143 | | RLTRNVRDLLDMLEEFEQYNVS | | | |
| | | | | | | | | | | FKSATEVFDTTSAIGKLFITMVG | | | |
| | | | | | | | | | | AMAEWERETIRERSLFGSRAAV | | | |
| | | | | | | | | | | REGNYIREAPFCYDNIEGKLHPN | | | |
| | | | | | | | | | | EYAKVIDLIVSMFKKGISANEIA | | | |
| | | | | | | | | | | RRLNSSKVHVPNKKSWNRNSLI | | | |
| | | | | | | | | | | RLMRSPVLRGHTKYGDMLIENT | | | |
| | | | | | | | | | | HEPVLSEHDYNAINNAISSKTHK | | | |
| | | | | | | | | | | SKVKHHAIFRGALVCPQCNRRL | | | |
| | | | | | | | | | | HLYAGTVKDRKGYKYDVRRY | | | |
| | | | | | | | | | | KCETCSKNKDVKNVSFNESEVE | | | |
| | | | | | | | | | | NKFVNLLKSYELNKFHIRKVEP | | | |
| | | | | | | | | | | VKKIEYDIDKINKQKINYTRSWS | | | |
| | | | | | | | | | | LGYIEDDEYFELMEEINATKKMI | | | |
| | | | | | | | | | | EEQTTENKQSVSKEQIQSINNFIL | | | |
| | | | | | | | | | | KGWEELTIKDKEELILSTVDKIE | | | |
| | | | | | | | | | | FNFIPKDKKHK | | | |
| | | | | | | | | | | TNTLDINNIHFKFS* | | | |

Sequences of insertion sites (i.e., recognition target sites) suitable for use in embodiments of the disclosure are presented below.

TABLE 11

| Bxb1_AttP_ | GTGGTTTGTCTGGTCAACCA | SEQ ID | TGGGTTTGTACCGTACACC | SEQ ID |
|---|---|---|---|---|
| GT_original_ | CCGCGGTCTCAGTGGTGTAC | NO: 394 | ACTGAGACCGCGGTGGTTG | NO: 473 |
| site | GGTACAAACCCA | | ACCAGACAAACCAC | |
| | | SEQ ID | | SEQ ID |
| Description | Forward Sequence (5′-3′) | NO: | Reverse Sequence (5′-3′) | NO: |
| Bxb1_AttP_ | GTGGTTTGTCTGGTCAACCA | 395 | TGGGTTTGTACCGTACACC | 474 |
| CG_site | CCGCGcgCTCAGTGGTGTAC | | ACTGAGCGCGCGGTGGTTG | |
| | GGTACAAACCCA | | ACCAGACAAACCAC | |
| Bxb1_AttP_ | GTGGTTTGTCTGGTCAACCA | 396 | TGGGTTTGTACCGTACACC | 475 |
| GC_site | CCGCGgcCTCAGTGGTGTAC | | ACTGAGGCCGCGGTGGTTG | |
| | GGTACAAACCCA | | ACCAGACAAACCAC | |
| Bxb1_AttP_ | GTGGTTTGTCTGGTCAACCA | 397 | TGGGTTTGTACCGTACACC | 476 |
| AT_site | CCGCGatCTCAGTGGTGTAC | | ACTGAGATCGCGGTGGTTG | |
| | GGTACAAACCCA | | ACCAGACAAACCAC | |
| Bxb1_AttP_ | GTGGTTTGTCTGGTCAACCA | 398 | TGGGTTTGTACCGTACACC | 477 |
| TA site | CCGCGtaCTCAGTGGTGTAC | | ACTGAGTACGCGGTGGTTG | |
| | GGTACAAACCCA | | ACCAGACAAACCAC | |
| Bxb1_AttP_ | GTGGTTTGTCTGGTCAACCA | 399 | TGGGTTTGTACCGTACACC | 478 |
| GG_site | CCGCGggCTCAGTGGTGTAC | | ACTGAGCCCGCGGTGGTTG | |
| | GGTACAAACCCA | | ACCAGACAAACCAC | |
| Bxb1_AttP_ | GTGGTTTGTCTGGTCAACCA | 400 | TGGGTTTGTACCGTACACC | 479 |
| TT_site | CCGCGttCTCAGTGGTGTACG | | ACTGAGAACGCGGTGGTTG | |
| | GTACAAACCCA | | ACCAGACAAACCAC | |
| Bxb1_AttP_ | GTGGTTTGTCTGGTCAACCA | 401 | TGGGTTTGTACCGTACACC | 480 |
| GA_site | CCGCGgaCTCAGTGGTGTAC | | ACTGAGTCCGCGGTGGTTG | |
| | GGTACAAACCCA | | ACCAGACAAACCAC | |
| Bxb1_AttP_ | GTGGTTTGTCTGGTCAACCA | 402 | TGGGTTTGTACCGTACACC | 481 |
| AG_site | CCGCGagCTCAGTGGTGTAC | | ACTGAGCTCGCGGTGGTTG | |
| | GGTACAAACCCA | | ACCAGACAAACCAC | |
| Bxb1_AttP_ | GTGGTTTGTCTGGTCAACCA | 403 | TGGGTTTGTACCGTACACC | 482 |
| CC_site | CCGCGccCTCAGTGGTGTAC | | ACTGAGGGCGCGGTGGTTG | |
| | GGTACAAACCCA | | ACCAGACAAACCAC | |
| Bxb1_AttP_ | GTGGTTTGTCTGGTCAACCA | 404 | TGGGTTTGTACCGTACACC | 483 |
| TC_site | CCGCGtcCTCAGTGGTGTAC | | ACTGAGGACGCGGTGGTTG | |
| | GGTACAAACCCA | | ACCAGACAAACCAC | |
| Bxb1_AttP_ | GTGGTTTGTCTGGTCAACCA | 405 | TGGGTTTGTACCGTACACC | 484 |
| CT_site | CCGCGctCTCAGTGGTGTAC | | ACTGAGAGCGCGGTGGTTG | |
| | GGTACAAACCCA | | ACCAGACAAACCAC | |
| Bxb1_AttP_ | GTGGTTTGTCTGGTCAACCA | 406 | TGGGTTTGTACCGTACACC | 485 |
| AA_site | CCGCGaaCTCAGTGGTGTAC | | ACTGAGTTCGCGGTGGTTG | |
| | GGTACAAACCCA | | ACCAGACAAACCAC | |
| Bxb1_AttP_ | GTGGTTTGTCTGGTCAACCA | 407 | TGGGTTTGTACCGTACACC | 486 |
| CA_site | CCGCGcaCTCAGTGGTGTAC | | ACTGAGTGCGCGGTGGTTG | |
| | GGTACAAACCCA | | ACCAGACAAACCAC | |
| Bxb1_AttP_ | GTGGTTTGTCTGGTCAACCA | 408 | TGGGTTTGTACCGTACACC | 487 |
| AC_site | CCGCGacCTCAGTGGTGTAC | | ACTGAGGTCGCGGTGGTTG | |
| | GGTACAAACCCA | | ACCAGACAAACCAC | |
| Bxb1_AttP_ | GTGGTTTGTCTGGTCAACCA | 409 | TGGGTTTGTACCGTACACC | 488 |
| TG_site | CCGCGtgCTCAGTGGTGTAC | | ACTGAGCACGCGGTGGTTG | |
| | GGTACAAACCCA | | ACCAGACAAACCAC | |
| Bxb1_AttB_ | GGCCGGCTTGTCGACGACG | 410 | CCGGATGATCCTGACGACG | 489 |
| 46_GT_ | GCGGTCTCCGTCGTCAGGAT | | GAGACCGCCGTCGTCGACA | |
| original_ | CATCCGG | | AGCCGGCC | |
| site | | | | |
| Bxb1_AttB_ | GGCCGGCTTGTCGACGACG | 411 | CCGGATGATCCTGACGACG | 490 |
| 46_AA_site | GCGaaCTCCGTCGTCAGGAT | | GAGTTCGCCGTCGTCGACA | |
| | CATCCGG | | AGCCGGCC | |
| Bxb1_AttB_ | GGCCGGCTTGTCGACGACG | 412 | CCGGATGATCCTGACGACG | 491 |
| 46_GA_site | GCGgaCTCCGTCGTCAGGAT | | GAGTCCGCCGTCGTCGACA | |
| | CATCCGG | | AGCCGGCC | |
| Bxb1_AttB_ | GGCCGGCTTGTCGACGACG | 413 | CCGGATGATCCTGACGACG | 492 |
| 46_CA_site | GCGcaCTCCGTCGTCAGGAT | | GAGTGCGCCGTCGTCGACA | |
| | CATCCGG | | AGCCGGCC | |
| Bxb1_AttB_ | GGCCGGCTTGTCGACGACG | 414 | CCGGATGATCCTGACGACG | 493 |
| 46_TA_site | GCGtaCTCCGTCGTCAGGATC | | GAGTACGCCGTCGTCGACA | |
| | ATCCGG | | AGCCGGCC | |
| Bxb1_AttB_ | GGCCGGCTTGTCGACGACG | 415 | CCGGATGATCCTGACGACG | 494 |
| 46_AG_site | GCGagCTCCGTCGTCAGGAT | | GAGCTCGCCGTCGTCGACA | |
| | CATCCGG | | AGCCGGCC | |
| Bxb1_AttB_ | GGCCGGCTTGTCGACGACG | 416 | CCGGATGATCCTGACGACG | 495 |
| 46_GG_site | GCGggCTCCGTCGTCAGGAT | | GAGCCCGCCGTCGTCGACA | |
| | CATCCGG | | AGCCGGCC | |
| Bxb1_AttB_ | GGCCGGCTTGTCGACGACG | 417 | CCGGATGATCCTGACGACG | 496 |
| 46_CG_site | GCGcgCTCCGTCGTCAGGAT | | GAGCGCGCCGTCGTCGACA | |
| | CATCCGG | | AGCCGGCC | |
| Bxb1_AttB_ | GGCCGGCTTGTCGACGACG | 418 | CCGGATGATCCTGACGACG | 497 |
| 46_TG_site | GCGtgCTCCGTCGTCAGGAT | | GAGCACGCCGTCGTCGACA | |
| | CATCCGG | | AGCCGGCC | |
| Bxb1_AttB_ | GGCCGGCTTGTCGACGACG | 419 | CCGGATGATCCTGACGACG | 498 |
| 46_AC_site | GCGacCTCCGTCGTCAGGAT | | GAGGTCGCCGTCGTCGACA | |
| | CATCCGG | | AGCCGGCC | |
| Bxb1_AttB_ | GGCCGGCTTGTCGACGACG | 420 | CCGGATGATCCTGACGACG | 499 |
| 46_GC_site | GCGgcCTCCGTCGTCAGGAT | | GAGGCCGCCGTCGTCGACA | |
| | CATCCGG | | AGCCGGCC | |
| Bxb1_AttB_ | GGCCGGCTTGTCGACGACG | 421 | CCGGATGATCCTGACGACG | 500 |
| 46_CC_site | GCGccCTCCGTCGTCAGGAT | | GAGGGCGCCGTCGTCGACA | |
| | CATCCGG | | AGCCGGCC | |
| Bxb1_AttB_ | GGCCGGCTTGTCGACGACG | 422 | CCGGATGATCCTGACGACG | 501 |
| 46_TC_site | GCGtcCTCCGTCGTCAGGATC | | GAGGACGCCGTCGTCGACA | |
| | ATCCGG | | AGCCGGCC | |
| Bxb1_AttB_ | GGCCGGCTTGTCGACGACG | 423 | CCGGATGATCCTGACGACG | 502 |
| 46_AT_site | GCGatCTCCGTCGTCAGGATC | | GAGATCGCCGTCGTCGACA | |
| | ATCCGG | | AGCCGGCC | |
| Bxb1_AttB_ | GGCCGGCTTGTCGACGACG | 424 | CCGGATGATCCTGACGACG | 503 |
| 46_CT_site | GCGctCTCCGTCGTCAGGATC | | GAGAGCGCCGTCGTCGACA | |
| | ATCCGG | | AGCCGGCC | |
| Bxb1_AttB_ | GGCCGGCTTGTCGACGACG | 425 | CCGGATGATCCTGACGACG | 504 |
| 46_TT_site | GCGttCTCCGTCGTCAGGATC | | GAGAACGCCGTCGTCGACA | |
| | ATCCGG | | AGCCGGCC | |
| Bxb1_AttB_ | GGCTTGTCGACGACGGCGG | 426 | ATGATCCTGACGACGGAGA | 505 |
| 38_GT_site | TCTCCGTCGTCAGGATCAT | | CCGCCGTCGTCGACAAGCC | |
| Bxb1_AttB_ | GGCTTGTCGACGACGGCGaa | 427 | ATGATCCTGACGACGGAGT | 506 |
| 38_AA_site | CTCCGTCGTCAGGATCAT | | TCGCCGTCGTCGACAAGCC | |
| Bxb1_AttB_ | GGCTTGTCGACGACGGCGga | 428 | ATGATCCTGACGACGGAGT | 507 |
| 38_GA_site | CTCCGTCGTCAGGATCAT | | CCGCCGTCGTCGACAAGCC | |
| Bxb1_AttB_ | GGCTTGTCGACGACGGCGca | 429 | ATGATCCTGACGACGGAGT | 508 |
| 38_CA_site | CTCCGTCGTCAGGATCAT | | GCGCCGTCGTCGACAAGCC | |
| Bxb1_AttB_ | GGCTTGTCGACGACGGCGta | 430 | ATGATCCTGACGACGGAGT | 509 |
| 38_TA_site | CTCCGTCGTCAGGATCAT | | ACGCCGTCGTCGACAAGCC | |
| Bxb1_AttB_ | GGCTTGTCGACGACGGCGag | 431 | ATGATCCTGACGACGGAGC | 510 |
| 38_AG_site | CTCCGTCGTCAGGATCAT | | TCGCCGTCGTCGACAAGCC | |
| Bxb1_AttB_ | GGCTTGTCGACGACGGCGgg | 432 | ATGATCCTGACGACGGAGC | 511 |
| 38_GG_site | CTCCGTCGTCAGGATCAT | | CCGCCGTCGTCGACAAGCC | |
| Bxb1_AttB_ | GGCTTGTCGACGACGGCGcg | 433 | ATGATCCTGACGACGGAGC | 512 |
| 38_CG_site | CTCCGTCGTCAGGATCAT | | GCGCCGTCGTCGACAAGCC | |
| Bxb1_AttB_ | GGCTTGTCGACGACGGCGtg | 434 | ATGATCCTGACGACGGAGC | 513 |
| 38_TG_site | CTCCGTCGTCAGGATCAT | | ACGCCGTCGTCGACAAGCC | |
| Bxb1_AttB_ | GGCTTGTCGACGACGGCGac | 435 | ATGATCCTGACGACGGAGG | 514 |
| 38_AC_site | CTCCGTCGTCAGGATCAT | | TCGCCGTCGTCGACAAGCC | |
| Bxb1_AttB_ | GGCTTGTCGACGACGGCGgc | 436 | ATGATCCTGACGACGGAGG | 515 |
| 38_GC_site | CTCCGTCGTCAGGATCAT | | CCGCCGTCGTCGACAAGCC | |
| Bxb1_AttB_ | GGCTTGTCGACGACGGCGcc | 437 | ATGATCCTGACGACGGAGG | \|516 |
| 38_CC_site | CTCCGTCGTCAGGATCAT | | GCGCCGTCGTCGACAAGCC | |
| Bxb1_AttB_ | GGCTTGTCGACGACGGCGtc | 438 | ATGATCCTGACGACGGAGG | 517 |
| 38_TC_site | CTCCGTCGTCAGGATCAT | | ACGCCGTCGTCGACAAGCC | |
| Bxb1_AttB_ | GGCTTGTCGACGACGGCGat | 439 | ATGATCCTGACGACGGAGA | 518 |
| 38_AT_site | CTCCGTCGTCAGGATCAT | | TCGCCGTCGTCGACAAGCC | |
| Bxb1_AttB_ | GGCTTGTCGACGACGGCGct | 440 | ATGATCCTGACGACGGAGA | 519 |
| 38_CT_site | CTCCGTCGTCAGGATCAT | | GCGCCGTCGTCGACAAGCC | |
| Bxb1_AttB_ | GGCTTGTCGACGACGGCGttC | 441 | ATGATCCTGACGACGGAGA | 520 |
| 38_TT_site | TCCGTCGTCAGGATCAT | | ACGCCGTCGTCGACAAGCC | |
| Cre Lox 66 | TACCGTTCGTATAATGTATG | 442 | ATAACTTCGTATAGCATAC | 521 |
| site | CTATACGAAGTTAT | | ATTATACGAACGGTA | |
| Cre Lox 71 | ATAACTTCGTATAATGTATG | 443 | TACCGTTCGTATAGCATAC | 522 |
| site | CTATACGAACGGTA | | ATTATACGAAGTTAT | |
| TP901-1 | TTTACCTTGATTGAGATGTT | 444 | CACAATTAACATCTCAATC | 523 |
| minimal | AATTGTG | | AAGGTAAA | |
| AttB site | | | | |
| TP901-1 | GCGAGTTTTTATTTCGTTTA | 445 | AAAGGAGTTTTTTAGTTAC | 524 |
| minimal | TTTCAATTAAGGTAACTAAA | | CTTAATTGAAATAAACGAA | |
| AttP site | AAACTCCTTT | | ATAAAAACTCGC | |
| PhiBT1 | CTGGATCATCTGGATCACTT | 446 | CAGGTTTTTGACGAAAGTG | 525 |
| minimal | TCGTCAAAAACCTG | | ATCCAGATGATCCAG | |
| AttB site | | | | |
| PhiBT1 | TTCGGGTGCTGGGTTGTTGT | 447 | TGGTGCTGAGTAGTTTCCC | 526 |
| minimal | CTCTGGACAGTGATCCATGG | | ATGGATCACTGTCCAGAGA | |
| AttP_site | GAAACTACTCAGCACCA | | CAACAACCCAGCACCCGAA | |
| Bacillus_ | gatatggggaagtgaatcagtac | 448 | ggtactgtggcggttgtactgat | 527 |
| cereus_AH1 | aaccgccacagtacc | | tcacttccccatatc | |
| 87_Int30_ | | | | |
| 38bp_Att | | | | |
| Staphylococ | tgggtggtacaggtgccacatta | 449 | cataaatggtacaactaatgtgg | 528 |
| cus_lugdun | gttgtaccatttatg | | cacctgtaccaccca | |
| ensis_N920 | | | | |
| 143_Int1 | | | | |
| 2_38bp_Att | | | | |
| Bacillus_ | gttgtttttccagatccagttgg | 450 | cttatatttacaggaccaactgg | 529 |
| cytotoxicus | tcctgtaaatataag | | atctggaaaaacaac | |
| NVH_391- | | | | |
| 98_Int13_3 | | | | |
| 8bp_Att | | | | |
| Bacillus_ | tggggaagtgaatcagtacaacc | 451 | ctgtggcggttgtactgattcac | 454 |
| cereus_AH1 | gccacag | | ttcccca | |
| 87_Int30_A | | | | |
| tt_30 | | | | |
| Bacillus_ | ggggaagtgaatcagtacaaccg | 452 | tgtggcggttgtactgattcact | 455 |
| cereus_AH1 | ccaca | | tcccc | |
| 87_Int30_A | | | | |
| tt_28 | | | | |
| Bacillus_ | gggaagtgaatcagtacaaccgc | 453 | gtggcggttgtactgattcactt | 456 |
| cereus_AH1 | cac | | ccc | |
| 87_Int30_A | | | | |
| tt_26 | | | | |
| Bacillus_ | ctgtggcggttgtactgattcac | 454 | tggggaagtgaatcagtacaacc | 451 |
| cereus_AH1 | ttcccca | | gccacag | |
| 87_Int30_A | | | | |
| tt_rc_30 | | | | |
| Bacillus_ | tgtggcggttgtactgattcact | 455 | ggggaagtgaatcagtacaaccg | 452 |
| cereus_AH187 | tcccc | | ccaca | |
| Int30_Att | | | | |
| rc_28 | | | | |
| Bacillus_ | gtggcggttgtactgattcactt | 456 | gggaagtgaatcagtacaaccgc | 453 |
| cereus_AH187 | ccc | | cac | |
| Int30_Att | | | | |
| rc_26 | | | | |
| Bacillus_ | tttttccagatccagttggtcct | 457 | tatttacaggaccaactggatct | 460 |
| cytotoxicus | gtaaata | | ggaaaaa | |
| NVH_391- | | | | |
| 98_Int13_A | | | | |
| tt_30 | | | | |
| Bacillus_ | ttttccagatccagttggtcct | 458 | atttacaggaccaactggatctg | 461 |
| cytotoxicus | gtaaat | | gaaaa | |
| NVH_391- | | | | |
| 98_Int13_A | | | | |
| tt_28 | | | | |
| Bacillus_ | tttccagatccagttggtcctgt | 459 | tttacaggaccaactggatctgg | 462 |
| cytotoxicus | aaa | | aaa | |
| NVH_391- | | | | |
| 98_Int13_A | | | | |
| tt_26 | | | | |
| Bacillus_ | tatttacaggaccaactggatct | 460 | tttttccagatccagttggtcct | 457 |
| cytotoxicus | ggaaaaa | | gtaaata | |
| NVH_391- | | | | |
| 98_Int13_A | | | | |
| tt_rc_30 | | | | |
| Bacillus_ | atttacaggaccaactggatct | 461 | ttttccagatccagttggtcctg | 458 |
| cytotoxicus | ggaaaa | | taaat | |
| NVH_391- | | | | |
| 98_Int13_A | | | | |
| tt_rc_28 | | | | |
| Bacillus_ | tttacaggaccaactggatctg | 462 | tttccagatccagttggtcctgt | 459 |
| cytotoxicus | gaaa | | aaa | |
| NVH_391- | | | | |
| 98_Int13_A | | | | |
| tt_rc_26 | | | | |
| N680429_ | CATTATATGTTTTTACAATC | 463 | cattatatgttcttacagtatgg | 530 |
| 560_31_50bp | CGGGCCGCCATACTGTAAG | | cggcccggattgtaaaaacatat | |
| | AACATATAATG | | aatg | |
| N191607_ | CGTTATAGGGTATTGCAGTA | 464 | cgttatagggtattacagtatgg | 531 |
| 8_101_50bp | CCGACCGCCATACTGTAATA | | cggtcggtactgcaataccctat | |
| | CCCTATAACG | | aacg | |
| N674992_ | TGTATCATTTTCATATAGTG | 465 | tgtatcattttcatatagttagc | 532 |
| 11308_50bp | TGCAGGTGCTAACTATATGA | | acctgcacactatatgaaaatga | |
| | AAATGATACA | | taca | |
| N684613_ | TGTCTACTATGTCTTTATGC | 466 | tgtctactatctgtatatgcgac | 533 |
| 54_96_50bp | CACATGTGTCGCATATACAG | | acatgtggcataaagacatagt | |
| | ATAGTAGACA | | agaca | |
| N252616_ | AATGAGGTCAGACGCATGG | 467 | catcgaccctgacgcatgcgga | 534 |
| 121_74_50bp | AGCGCCGCCTCCGCATGCGT | | ggcggcgctccatgcgtctgacc | |
| | CAGGGTCGATG | | tcatt | |
| N683040_ | GTTAGTACCCAAATGATAA | 468 | gttagtacccaaatgacaaaagg | 535 |
| 222_19_50bp | AAGGATGACCTTTTGTCATT | | tcatccttttatcatttgggtac | |
| | TGGGTACTAAC | | taac | |
| N687537_ | GTTTATAAAACCGATGCCGC | 469 | cttattaaaacccgttccgcttc | 536 |
| 173_59_50bp | TTTGACAGAAGCGGAACGG | | tgtcaaagcggcatcggttttat | |
| | GTTTTAATAAG | | aaac | |
| N183629_ | GGCCGCGAGGTCGTGTTCGT | 470 | ggcgtgatggtcgtgaacctcaa | 537 |
| 47_40_50b_p | CGTCATGTTGAGGTTCACGA | | catgacgacgaacacgacctcg | |
| | CCATCACGCC | | cggcc | |
| N191533_ | TATAAACTGATATAATTCAA | 471 | tctacatcttgaatatatcaagt | 538 |
| 224_76_50bp | AGTTATAACTTGATATATTC | | tataactttgaattatatcagtt | |
| | AAGATGTAGA | | tata | |
| N682356_ | TATTATATCTAAAAGCAGTA | 472 | aattatatctaaaagcactaag | 539 |
| 188_20_50 | TGGCGGAGCTTAGTGCTTTT | | ctccgccatactgcttttagat | |
| bp | AGATATAATT | | ataata | |

6.9. Nucleic acid construct design

A single nucleic acid construct is described herein that allows for programmable gene insertion (PGI) (e.g., incorporation of any template into any DNA locus using DNA delivery of a single component DNA).

In various embodiments, the nucleic acid construct contains a nucleotide sequence encoding an integrase, a nucleotide sequence encoding a prime editor fusion protein or a gene writer protein, a nucleotide sequence encoding at least a first attachment site-containing guide RNA (atgRNA), a DNA donor template (i.e., “cargo”), optionally a nucleotide sequence encoding a nickase guide RNA (ngRNA), and optionally a nucleotide sequence encoding a recombinase.

In various embodiments, the nucleic acid construct contains a nucleotide sequence encoding an integrase, a nucleotide sequence encoding a prime editor fusion protein or a gene writer protein, a nucleotide sequence encoding at least a first attachment site-containing guide RNA (atgRNA), a DNA donor template (i.e., “cargo”), a nucleotide sequence encoding a nickase guide RNA (ngRNA), and optionally a nucleotide sequence encoding a recombinase. In various embodiments, the nucleic acid construct contains a nucleotide sequence encoding an integrase, a nucleotide sequence encoding a prime editor fusion protein or a gene writer protein, a nucleotide sequence encoding at least a first attachment site-containing guide RNA (atgRNA), a DNA donor template (i.e., “cargo”), a nucleotide sequence encoding a nickase guide RNA (ngRNA), and a nucleotide sequence encoding a recombinase. In various embodiments, the nucleic acid construct contains a nucleotide sequence encoding an integrase, a nucleotide sequence encoding a prime editor fusion protein or a gene writer protein, a nucleotide sequence encoding a first attachment site-containing guide RNA (atgRNA), a second attachment site-containing guide RNA (atgRNA), a DNA donor template (i.e., “cargo”), and a nucleotide sequence encoding a recombinase, where the first atgRNA and the second atgRNA are an at least first pair of atgRNAs. In various embodiments, the nucleic acid construct contains a nucleotide sequence encoding an integrase, a nucleotide sequence encoding a prime editor fusion protein or a gene writer protein, a nucleotide sequence encoding a first attachment site-containing guide RNA (atgRNA), a nucleotide sequence encoding a second attachment site-containing guide RNA (atgRNA), and a DNA donor template (i.e., “cargo”), where the first atgRNA and the second atgRNA are an at least first pair of atgRNAs.

In various embodiments, the nucleic acid construct comprises: a nucleotide sequence encoding a prime editor fusion protein; a nucleotide sequence encoding at least a first attachment site-containing guide RNA (atgRNA); a nucleotide sequence encoding a recombinase; a nucleic acid cargo; and a nucleotide sequence encoding a nickase guide RNA (ngRNA).

In some embodiments, the nucleic acid construct comprises: a nucleotide sequence encoding a prime editor fusion protein, a nucleotide sequence encoding a first attachment site-containing guide RNA (atgRNA), a nucleotide sequence encoding a second attachment site-containing guide RNA (atgRNA), and a nucleotide sequence encoding a recombinase; a nucleic acid cargo; where the first atgRNA and the second atgRNA are an at least first pair of atgRNAs.

In some embodiments, a single promoter drives expression of all the different nucleotide sequences on the single nucleic acid construct. In some embodiments, two or more promoters drive expression of the different nucleotide sequences on the single nucleic acid construct. In typical embodiments, at least one promoter drives the expression of the prime editor fusion protein or the gene writer protein, atgRNA, optionally ngRNA, integrase (e.g., serine integrase), and optionally recombinase. In some embodiments, the promoter is an immediate early promoter such as a CMV promoter or a type III RNA polymerase III promoter such as a U6 promoter. In some embodiments, the promoter is any Pol II promoter. In some embodiments, the atgRNA and ngRNA are driven by any Pol III promoter. In some embodiments, the respective promoters used to drive the expression of the protein components, the atgRNA, and the ngRNA have different promoter expression strength, fidelity, selectivity, and/or tissue-specificity.

In various embodiments, the integrase that is encoded in the nucleic acid construct is fused to the prime editor fusion protein or the Gene Writer protein optionally by a linker. In various embodiments, the recombinase that is encoded in the nucleic acid construct is fused to the prime editor fusion protein or the Gene Writer protein optionally by a linker.

In some embodiments, the nucleic acid construct contains a 5′ inverted terminal repeat (ITR). In some embodiments, the nucleic acid construct contains a 3′ inverted terminal repeat (ITR). In some embodiments, the nucleic acid construct contains a 5′ and a 3′ inverted terminal repeat. In some embodiments, the 5′ and 3′ ITR are not derived from the same serotype of virus. In some embodiments, the ITRs are derived from Adenovirus, AAV2, AAV5, or both.

In typical embodiments, the nucleic acid construct further comprises at least one integrase recognition target site (e.g., an integrase recognition site in the nucleic acid construct used to facilitate integration of all or part of the nucleic acid construct into an integrase recognition site incorporated into a cell genome). In such cases, the at least one integrase recognition site is separate from the integration sequences encoded by the first atgRNA, second atgRNA, or both. In some embodiments, the at least one integrase recognition site is a cognate pair with the integration sequences encoded by the first atgRNA, second atgRNA, or by a combination of the first atgRNA and second atgRNA. In some embodiments, the at least one integrase recognition site is specific for a B×B1, B. cereus (BceINTc or Bcec), N191352_143_72 stool sample from China (SscINTd or Sscd), N684346_90_69 stool sample from adult in China (SacINTd or Sacd).

In certain embodiments, the nucleic acid construct further comprises at least one recombinase recognition target site (e.g., one recombinase recognition site, two recombination recognition sites, three recombinase recognition sites, or four recombinase recognitions site, or more). In some embodiments, the at least one recombinase recognition site is specific for a FLP, a FLP mutant, Cre, or a Cre mutant. In some embodiments, the nucleic acid construct comprises two recombinase recognition sites where the two sites flank the nucleic acid cargo. In such cases, the two recombinase recognition sites are capable of self-circularizing to form a circular construct when contacted with a recombinase.

In certain embodiments, the nucleic acid construct further comprises at least one recombinase recognition target site and at least one integrase recognition target site.

In typical embodiments, the nucleic acid construct contains a nucleic acid cargo (i.e., “integration” cargo) of interest. In some embodiments, the nucleic acid cargo is one or more genes or gene fragments. In some embodiments, the nucleic acid cargo is at least one intron, at least one exon sequence, or a combination thereof. In some embodiments, the nucleic acid cargo is at least one intron fragment, at least exon fragment sequence, or a combination thereof. In some embodiments the nucleic acid cargo is an expression cassette. In some embodiments, the nucleic acid cargo is a logic gate or logic gate system. The logic gate or logic gate system may be DNA based, RNA based, protein based, or a mix of DNA, RNA, and protein. In some embodiments, the nucleic acid cargo is DNA or RNA. In some embodiments, the nucleic acid cargo is a genetic, protein, or peptide tag and/or barcode.

In certain embodiments, the constructs and methods described herein may be utilized for monitoring a biological or biochemical cellular condition or circuits, such as pH via a marker. In some embodiments, the constructs and methods described herein may be utilized for recording, via writing directly to a genome or intracellular DNA element, cellular, environmental, chemical, or other cellular temporal or spatial related events. In some embodiments, the constructs and methods described herein may be utilized for recording, via writing directly to a genome or intracellular DNA element, cellular lineage information.

In certain embodiments, the genome to be programmably inserted into is eukaryotic or porkarytotic. In certain embodiments, the genome is mammalian, nonmammalian, human, murine, or NHP.

In additional embodiments, constructs and methods describe herein may be utilized in agricultural settings for production of crops with improved properties or traits as well as to produce livestock, such as cattle, avian, or other species with improved or desirable features.

### 6.10. Integrase-or Recombinase-Mediated Self-Circularization of a Subsequence of the Single Nucleic Acid Construct

In some embodiments, the single nucleic acid construct comprises a sub-sequence of the nucleic acid construct that is capable of self-circularizing to form a self-circular nucleic acid. In some embodiments, the single nucleic acid construct comprises a physical portion or region of the nucleic acid construct that is capable of self-circularizing to form a circular construct. As used herein, the term “sub-sequence” refers to a portion of the single nucleic acid construct that is capable of self-circularizing, where the subsequence is flanked by integrase recognition sites or recombinase recognition sites positioned to enable self-circularization. As used herein, the term “self-circular nucleic acid” refers to a double-stranded, circular nucleic acid construct produced as a result of recombination of a cognate pair of integrase or recombinase recognition sites present on the single nucleic acid construct. Recombination occurs when the single nucleic acid construct is contacted with an integrase or a recombinase under conditions that allow for recombination of the cognate pair or integrase or recombinase recognition sites.

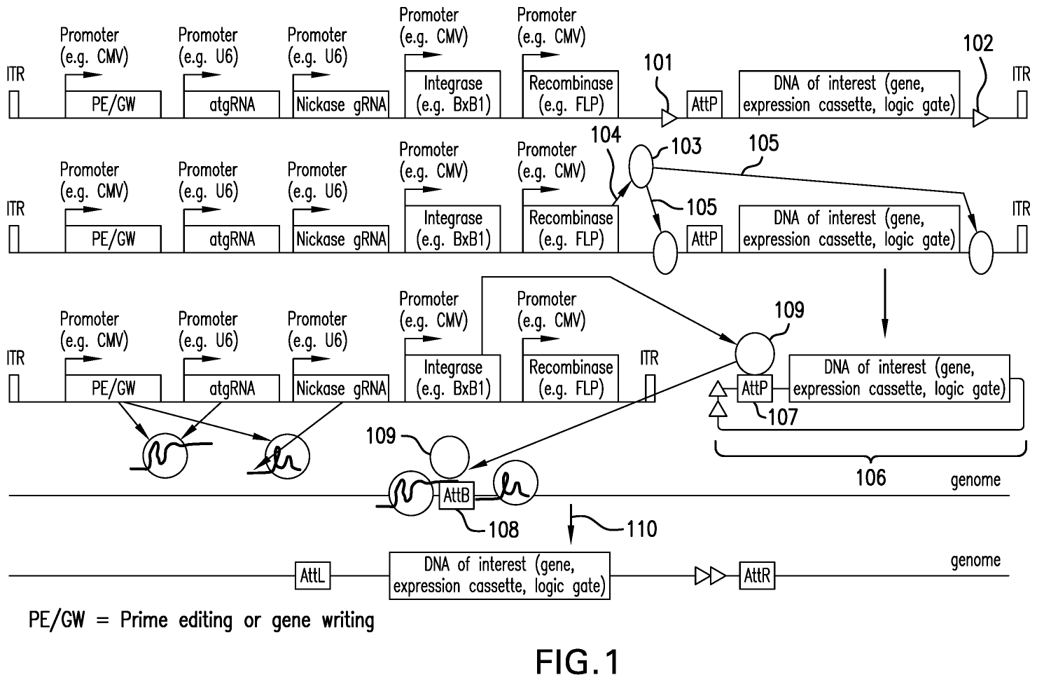

In some embodiments, the sub-sequence of the single nucleic acid construct includes a first recombinase recognition site and a second recombinase recognition site, wherein the first and second recombinase recognition sites are capable of being recombined by a recombinase. In some embodiments, the sub-sequence of the single nucleic acid includes a first recombinase recognition site, a second recombinase recognition site, and an integrase recognition site (e.g., a second integrase recognition site), where the first and second recombinase recognition sites flank the integrase recognition site. In such cases, the first recombinase recognition site, the second recombinase recognition, and a recombinase enable the self-circularizing and formation of the circular construct (see, e.g., FIG. 1).

In some embodiments, the sub-sequence of the single nucleic acid construct includes a third integrase recognition site and a fourth integrase recognition site, wherein the third and fourth integrase recognition sites are a cognate pair. In some embodiments, the subsequence of the single nucleic acid construct includes the second integrase recognition site, the third integrase recognition site, the fourth integrase recognition site, where the third and fourth integrase recognition sites flank the second integrase. In such cases, the third integrase recognition site, the fourth integrase recognition site, and an integrase enable self-circularization and formation of the circular construct. In such cases, the third integrase recognition site and/or the fourth integrase recognition sites cannot recombine due, in part, to having different central dinucleotides with the first integrase recognition site and/or the second integrase recognition site.

In some embodiments where the subsequence includes three or more integrase recognition sites, each integrase recognition site or each pair of integrase recognition is capable of being recognized by a different integrase. In some embodiments where the subsequence includes three or more integrase recognition sites, each integrase recognition site or each pair of integrase recognition comprises a different central dinucleotide.

In some embodiments, self-circularizing is mediated at the integrase recognition sites or recombinase recognition sites. In some embodiments, the self-circularizing is mediated by an integrase or a recombinase.

In some embodiments, upon introducing the nucleic acid construct into a cell and after self-circularizing to form the self-circular nucleic acid, the self-circular nucleic acid comprising the second integrase recognition site is capable of being integrated into the cell's genome at the target sequence that contains the first integrase recognition site.

In some embodiments, following self-circularization, the self-circular nucleic acid comprises one or more additional integrase recognition sites that enable integration of an additional nucleic acid cargo. In such cases, the additional nucleic acid cargo includes a sequence that is a cognate pair with one or more of the additional integrase recognition sites in the self-circular nucleic acid. For example, integration of the self-circular nucleic acid into the genome of a cell results in integration of the one or more integrase recognition sites into the genome along with the nucleic acid cargo. The integrated one or more integrase recognition sites serve as an integrase recognition site (beacon) for placing the additional nucleic acid cargo. Upon contacting the cell harboring the integrated nucleic acid cargo and the one or more additional integrase recognition sites with an integrase and the second additional nucleic that includes a sequence that is an integration cognate to the one or more integrase recognition sites, thereby integrating the additional nucleic acid cargo.

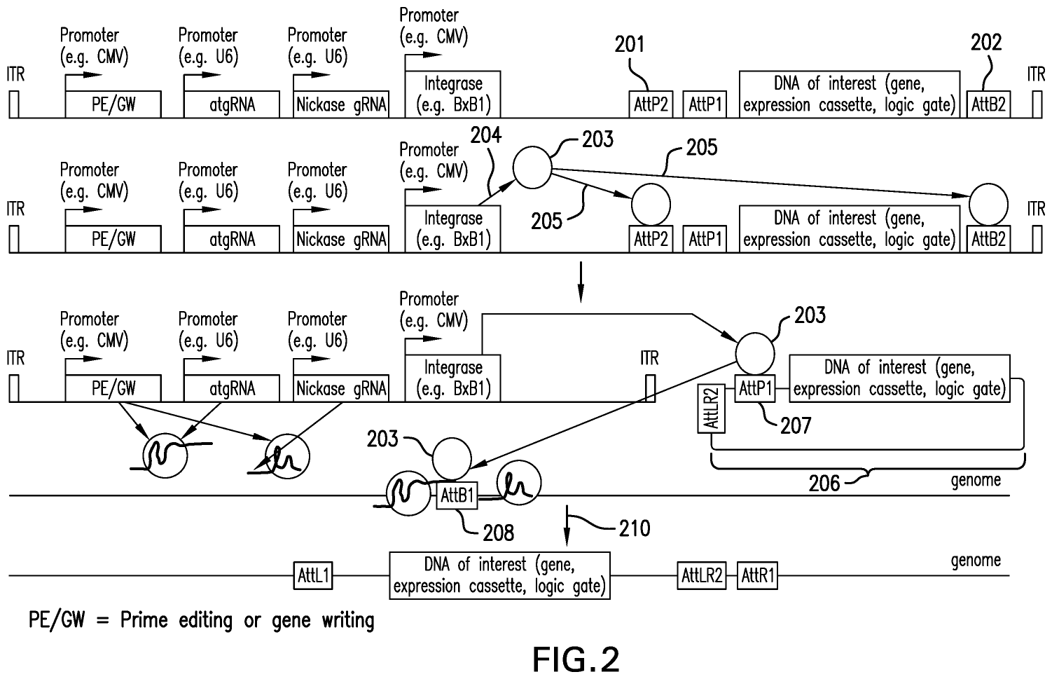

In some embodiments, the self-circular nucleic acid includes a second integrase recognition stie that is capable of being integrated into a genomic locus that contains the first integrase recognition site (i.e., the first and second integrase recognition sites are a cognate pair). See, FIGS. 1-2.

In some embodiments, the single nucleic acid construct comprises two recombinase recognition sites where the two sites flank the nucleic acid cargo. In such cases, the two recombinase recognition sites are capable of self-circularizing to form a self-circular nucleic acid when contacted with a recombinase. FIG. 1 illustrates a non-limiting example of a single nucleic acid construct that includes two recombinase recognition sites capable of self-circularizing to form a circular construct (e.g., a self-circular nucleic acid) when contacted with a recombinase. In FIGS. 1, 101 and 102 are recombinase recognition sites present in the single nucleic acid construct. The single nucleic acid construct also includes a sequence encoding a recombinase 103. The recombinase 103 is expressed 104 and contacts 105 the recombinase recognition sites (101 and 102), thereby mediating self-circularization of a portion of the single nucleic acid construct and producing a self-circular nucleic acid 106.

In some embodiments, the self-circular nucleic acid 106 includes a sequence 107 that is an integration cognate (e.g., a cognate pair) to the first integrase recognition sequence 108. In such cases, the self-circular nucleic acid is integrated into a genome at the incorporation stie of the first integrase recognition site. In some embodiments, integration of the self-circular nucleic acid into the genome is mediated by an integrase. For example, FIG. 1 illustrates a non-limiting example where the single nucleic acid construct also includes a sequence encoding an integrase 109. The integrase 109 is expressed and integrates 110 the circular construct 106 into the first integrase recognition site 108 site-specifically incorporated into the genome.

In some embodiments, the nucleic acid construct comprises two integrase recognition sites where the two sites flank the nucleic acid cargo. In such cases, the two integrase recognition sites are capable of self-circularizing to form a self-circular nucleic acid when contacted with an integrase. FIG. 2 illustrates a non-limiting example of a single nucleic acid construct that includes two integration sequences capable of self-circularizing to form a circular construct (e.g., a self-circular nucleic acid) when contacted with a recombinase. In FIGS. 2, 201 and 202 are integrase recognition sites (e.g., the third and fourth integrase recognition sites) present in the single nucleic acid construct. The single nucleic acid construct also includes a sequence encoding an integrase 203. The integrase 203 is expressed 204 and contacts 205 the integrase recognition sites (201 and 202), thereby mediating self-circularization of a portion of the single nucleic acid construct and producing a self-circular nucleic acid 206.

In some embodiments, the self-circular nucleic construct 206 includes a sequence 207 that is a cognate pair to the site-specifically incorporated integration sequence 208. As shown in FIG. 2, one embodiment uses the same integrase for both self-circularizing and integration of the self-circular nucleic acid. The integrase 203 is expressed 204 and integrates 210 the self-circular nucleic acid 206 into the first integrase recognition site 208 site-specifically incorporated into the genome.

High efficiency and/or fast integrase recognition target sites allow for integrase-mediated template circularization to happen prior to integrase-mediated genomic integration at an integrase recognition target site within the genome (i.e. “beacon” or “landing pad”). In some embodiments, the integration rate can be altered by changing the dinucleotide used within the integrase recognition target site. In some embodiments, the integration rate can be altered by changing the integrase recognition target site sequence length. In some embodiments, the integration rate can be altered by changing the dinucleotide used within the integrase recognition target site and by changing the integrase recognition target site sequence length. For example, the attB/attP integrase recognition target site sequence length can be about 32-46 bp in length. In some embodiments, high efficiency and/or fast integrase target recognition is mediated by orthogonal integrases or recombinases.

In some embodiments where a single nucleic acid construct includes a first cognate pair (e.g., a first integrase recognition site and a second integrase recognition site) and a second cognate pair (e.g., a third integrase recognition site and a fourth recognition site), the first cognate pair and the second cognate pair are designed such that each cognate pair has a different integration rate. In such embodiments, the cognate pair with the faster integration rate recombines prior to the cognate pair with the slower integration rate. For example, as shown in FIG. 2, the first cognate pair is represented by 207 and 208 and the second cognate pair is represented by 201 and 202. In one embodiment of the illustration in FIG. 2, the second cognate pair (i.e., 201 and 202) has a faster integration rate whereby self-circularization occurs prior to integration into the genome.

In some embodiments, the self-circularizing is effected at an integrase or recombinase recognition target sequence. In typical embodiments, the self-circularizing is mediated by an integrase or a recombinase.

In typical embodiments, the self-circularized nucleic acid comprises a DNA cargo. embodiments, the DNA cargo is a gene or gene fragment. In some embodiments the DNA cargo is an expression cassette. In some embodiments, the DNA cargo is a logic gate or logic gate system. The logic gate or logic gate system may be DNA based, RNA based, protein based, or a mix of DNA, RNA, and protein. In some embodiments, the nucleic acid cargo is a genetic, protein, or peptide tag and/or barcode.

In some embodiments, the DNA cargo contains one or more orthogonal recombinase recognition target site(s). In some embodiments, the DNA cargo contains one or more orthogonal integrase recognition target site(s). The region that contains one or more orthogonal recombinase or integrase recognition target site(s) may be referred to as a multiple access site. Further, after DNA cargo integration into a genomic locus, the additional one or more orthogonal recombinase or integrase target recognition site(s) contained within the inserted DNA cargo may be subsequently targeted via a recombinase or integrase to incorporate additional DNA cargo. The DNA cargo may contain one or one or more orthogonal recombinase or integrase target recognition site(s). Hence, because each newly genomically incorporated DNA template, insert, or DNA cargo, may contain at least one “embedded” or “nested” orthogonal recombinase or integrase target recognition site(s) it becomes possible to programmatically (spatially and temporally) access, introduce, delete, and modify a genomic-or DNA-locus of interest at the orthogonal recombinase or integrase target recognition site(s).

In typical embodiments, the self-circular nucleic acid is capable of being integrated into a genomic locus that contains an integrase or recombinase recognition site (i.e., “beacon” or “landing pad” site). In typical embodiments, the self-circular nucleic acid contains the DNA cargo of interest. In some embodiments, the integrase or recombinase that mediates self-circularization is fused or linked to the prime editor protein fusion.

In typical embodiments, the nucleic acid construct that contains a nucleotide sequence encoding an integrase, encoding a prime editor fusion protein or a gene writer protein, a nucleotide sequence encoding one or more attachment site-containing guide RNA (atgRNA), optionally a nucleotide sequence encoding a nickase guide RNA (ngRNA), a nucleotide sequence encoding an integrase, a DNA cargo, and optionally a nucleotide sequence encoding a recombinase is vectorized.

In some embodiments, an integration target recognition site is incorporated (i.e., beacon placement) into a human primary cell genome using a single atgRNA and a single nicking guide RNA (ngRNA). In some embodiments, an integration target recognition site is incorporated into a human primary cell genome using two atgRNAs (dual or paired or twin atgRNAs). In certain embodiments, the nucleic acid construct comprises two atgRNAs.

In some embodiments, the atgRNA reverse transcriptase template encodes for a first single-stranded DNA sequence (i.e., a first DNA flap) that contains a complementary region to a second single-stranded DNA sequence (i.e., a second DNA flap) encoded by a second atgRNA comprised of a reverse transcriptase template. In certain embodiments, the complementary region between the first and second single-stranded DNA sequences is comprised of more than 10 consecutive bases of an integrase target recognition site. In certain embodiments, the complementary region between the first and second single-stranded DNA sequences is comprised of more than 20 consecutive bases of an integrase target recognition site. In certain embodiments, the complementary region between the first and second single-stranded DNA sequences is comprised of more than 30 consecutive bases of an integrase target recognition site. Use of two guide RNAs that are (or encode DNA that is) partially complementary to each other and comprised of consecutive bases of an integrase target recognition site are referred to as dual, paired, annealing, complementary, or twin attachment site-containing guide RNAs (atgRNAs).

### 6.11. Genes and Targets

This disclosure provides compositions and methods for correcting or replacing genes or gene fragments (including introns or exons) or inserting genes in new locations. In certain embodiments, such a method comprises recombination or integration into a safe harbor site (SHS). A frequently used human SHS is the AAVS1 site on chromosome 19q, initially identified as a site for recurrent adeno-associated virus insertion. Another locus comprises the human homolog of the murine Rosa26 locus. Yet another SHS comprises the human H11 locus on chromosome 22. In some cases, a complete gene may be prohibitively large and replacement of an entire gene impractical. In certain embodiments, a method of the invention comprises recombining corrective gene fragments into a defective locus.

The methods and compositions can be used to target, without limitation, stem cells for example induced pluripotent stem cells (iPSCs), HSCs, HSPCs, mesenchymal stem cells, or neuronal stem cells and cells at various stages of differentiation. In certain embodiments, methods and compositions of the invention are adapted to target organoids, including patient derived organoids. In certain embodiments, methods and compositions of the invention are adapted to treat muscle cells, not limited to cardiomyocytes for Duchene Muscular Dystrophy (DMD). The dystrophin gene is the largest gene in the human genome, spanning ˜2.3 Mb of DNA. DMD is composed of 79 exons resulting in a 14-kb full-length mRNA. Common mutations include mutations that disrupt the reading frame of generate a premature stop codon. An aspect of DMD that lends it to gene editing as a therapeutic approach is the modular structure of the dystrophin protein. Redundancy in the central rod domain permits the deletion of internal segments of the gene that may harbor loss-of-function mutations, thereby restoring the open reading frame (ORFs). The following are non-limiting diseases that may be treated utilizing the methods and compositions of the present disclosure:

### Inherited Retinal Diseases:

Stargardt Disease (ABCA4)
Leber congenital amaurosis 10 (CEP290)
X linked Retinitis Pigmentosa (RPGR)
Autosomal Dominant Retinitis Pigmentosa (RHO)

### Liver Diseases:

Wilson's disease (ATP7B)
Alpha-1 antitrypsin (SERPINA1)

### Intellectual Disabilities:

Rett Syndrome (MECP2)
SYNGAP1-ID (SYNGAP1)
CDKL5 deficiency disorder (CDKL5)

### Peripheral Neuropathies:

Charcot-Marie-Tooth 2A (MFN2)

### Lung Diseases:

Cystic Fibrosis (CFTR)
Alpha-1 Antitrypsin (SERPINA1)

### Blood Disorders:

Sickle Cell
Hemophilia,
Factor VIII or
Factor IX
CFTR (cystic fibrosis transmembrane conductance regulator)

Over 2500 mutations have been identified associated with various diseases and defects.

The most common cystic fibrosis (CF) mutation F508del removes a single amino acid. In some embodiments, recombining human CFTR into an SHS of a cell that expresses CFTR F508del is a corrective treatment path. In certain embodiments, appropriate cells include epithelial cells which may be derived from iPSCs. Proposed validation is detection of persistent CFTR mRNA and protein expression in transduced cells.

Sickle cell disease (SCD) is caused by mutation of a specific amino acid-valine to glutamic acid at amino acid position 6. In some embodiments, SCD is corrected by recombination of the HBB gene into a safe harbor site (SHS) and by demonstrating correction in a proportion of target cells that is high enough to produce a substantial benefit. Appropriate test cells include erythroid cells which may be derived from iPSCs. In some embodiments, validation is detection of persistent HBB mRNA and protein expression in transduced cells.

### DMD-Duchenne Muscular Dystrophy

The dystrophin gene is the largest gene in the human genome, spanning ˜2.3 Mb of DNA. DMD is composed of 79 exons resulting in a 14-kb full-length mRNA. Common mutations include mutations that disrupt the reading frame of generate a premature stop codon. An aspect of DMD that lends it to gene editing as a therapeutic approach is the modular structure of the dystrophin protein. Redundancy in the central rod domain permits the deletion of internal segments of the gene that may harbor loss-of-function mutations, thereby restoring the open reading frame (ORFs).

In some embodiments, recombination will be into safe harbor sites (SHS). A frequently used human SHS is the AAVS1 site on chromosome 19q, initially identified as a site for recurrent adeno-associated virus insertion. In some embodiments, the site is the human homolog of the e murine Rosa26 locus (pubmed.ncbi.nlm.nih.gov/18037879). In some embodiments, the site is the human H11 locus on chromosome 22. Proposed target cells for recombination include stem cells for example induced pluripotent stem cells (iPSCs) and cells at various stages of differentiation. In some cases, a complete gene may be prohibitively large and replacement of an entire gene impractical. In such instances, rescuing mutants by recombining in corrected gene fragments with the methods and systems described herein is a corrective option.

In some embodiments, correcting mutations in exon 44 (or 51) by recombining in a corrective coding sequence downstream of exon 43 (or 50), using the methods and systems described herein is a corrective option. Appropriate test target cells include cardiomyocytes derived from iPSCs. Proposed validation is detection of persistent DMD mRNA and protein expression in transduced cells.

### F8 (Factor VIII)

A large proportion of severe hemophilia A patients harbor one of two types of chromosomal inversions in the FVIII gene. The recombinase technology and methods described herein are well suited to correcting such inversions (and other mutations) by recombining of the FVIII gene into a SHS.

In some embodiments, correcting factor VIII deficiency by recombining the FVIII gene into an SHS is a corrective path. Appropriate test target cells include liver cells and endothelial cells which may be derived from iPSCs. Proposed validation is detection of persistent FVIII mRNA and protein expression in transduced cells.

### 6.12. Methods of Treatment

In another aspect, methods of treatment are presented. The method comprises administering an effective amount of the pharmaceutical composition comprising the nucleic acid construct or vectorized nucleic acid construct described above to a patient in need thereof.

DNA or RNA viral vectors can be administered directly to patients (in vivo) or they can be used to treat cells in vitro, and the modified cells may optionally be administered to patients (ex vivo). Conventional viral based systems to be used herein could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Methods of non-viral delivery of the single nucleic acid construct described herein include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

### 6.12.1.1 Lipid Nanoparticle Delivery

In some embodiments, the single nucleic acid construct is packaged in a LNP and administered intravenously. In some embodiments, the single nucleic acid construct is packaged in a LNP and administered intrathecally. In some embodiments, the single nucleic acid construct is packaged in a LNP and administered by intracerebral ventricular injection. In some embodiments, the single nucleic acid construct is packaged in a LNP and administered by intracisternal magna administration. In some embodiments, the single nucleic acid construct is packaged in a LNP and administered by intravitreal injection.

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

In another embodiment, LNP doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

The charge of the LNP must be taken into consideration. As cationic lipids combined with negatively charged lipids to induce nonbilayer structures that facilitate intracellular delivery. Because charged LNPs are rapidly cleared from circulation following intravenous injection, ionizable cationic lipids with pKa values below 7 were developed (see, e.g., Rosin et al, Molecular Therapy, vol. 19, no. 12, pages 1286-220 Dec. 2011). Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). It has been shown that LNP siRNA systems containing these lipids exhibit remarkably different gene silencing properties in hepatocytes in vivo, with potencies varying according to the series DLinKC2-DMA>DLinKDMA>DLinDMA>>DLinDAP employing a Factor VII gene silencing model (see, e.g., Rosin et al, Molecular Therapy, vol. 19, no. 12, pages 1286-220 Dec. 2011). A dosage of 1 μg/ml of LNP in or associated with the LNP may be contemplated, especially for a formulation containing DLinKC2-DMA.

In some embodiments, the LNP composition comprises one or more one or more ionizable lipids. As used herein, the term “ionizable lipid” has its ordinary meaning in the art and may refer to a lipid comprising one or more charged moieties. In some embodiments, an ionizable lipid may be positively charged or negatively charged. In principle, there are no specific limitations concerning the ionizable lipids of the LNP compositions disclosed herein. In some embodiments, the one or more ionizable lipids are selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanami-ne (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3)-cholest-5-en-3-yloxy]octyl}oxy)-N,N-dimethyl-3-[(9Z,12Z)-octad-eca-9,12-dien-1-yloxy]propan-1-amine (Octyl-CLinDMA), (2R)-2-({8-[(3)-cholest-5-en-3-yloxy]octyl}oxy)-N,N-dimethyl-3-[(9Z, 12Z)--octadeca-9,12-dien-1-yloxy]propan-1-amine (Octyl-CLinDMA (2R)), and (2S)-2-({8-[(3)-cholest-5-en-3-yloxy]octyl}oxy)-N,N-dimethyl-3-[(9Z,12Z)--octadeca-9,12-dien-1-y loxy]propan-1-amine (Octyl-CLinDMA (2S)). In one embodiment, the ionizable lipid may be selected from, but not limited to, an ionizable lipid described in International Publication Nos. WO2013086354 and WO2013116126.

In some embodiments, the lipid nanoparticle may include one or more (e.g., 1, 2, 3, 4, 5, 6, 7, or 8) cationic and/or ionizable lipids. Such cationic and/or ionizable lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanami-ne (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 2-({8-[(3.beta.)-cholest-5-en-3-yloxy]octyl}oxy)-N,N-dimethyl-3-[(9Z,12Z)--octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3Bcholest-5-en-3-yloxy]octyl}oxy)-N,N-dimethyl-3-[(9Z-,12Z)-octadeca-9,12-dien-1-yloxy]propan-1-amine (Octyl-CLinDMA (2S)).N,N-dioleyl-N,N-dimethylammonium chloride (“DODAC”); N-(2,3-dioleyloxy) propyl-N,N-N-triethylammonium chloride (“DOTMA”); N,N-distearyl-N,N-dimethylammonium bromide (“DDAB”); N-(2,3-dioleoyloxy) propyl)-N,N,N-trimethylammonium chloride (“DOTAP”); 1,2-Dioleyloxy-3-trimethylaminopropane chloride salt (“DOTAP.Cl”); 3-.beta.-(N--(N′, N′-dimethylaminoethane)-carbamoyl) cholesterol (“DC-Chol”), N-(1-(2,3-dioleyloxy) propyl)-N-2-(sperminecarboxamido)ethyl)-N,N-dimethyl--ammonium trifluoracetate (“DOSPA”), dioctadecylamidoglycyl carboxyspermine (“DOGS”), 1,2-dioleoyl-3-dimethylammonium propane (“DODAP”), N,N-dimethyl-2,3-dioleyloxy) propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic and/or ionizable lipids can be used, such as, e.g., LIPOFECTIN.RTM. (including DOTMA and DOPE, available from GIBCO/BRL), and LIPOFECTAMINE.RTM. (including DOSPA and DOPE, available from GIBCO/BRL). KL10, KL22, and KL25 are described, for example, in U.S. Pat. No. 8,691,750.

In some embodiments, the LNP composition comprises one or more amino lipids. The terms “amino lipid” and “cationic lipid” are used interchangeably herein to include those lipids and salts thereof having one, two, three, or more fatty acid or fatty alkyl chains and a pH-titratable amino head group (e.g., an alkylamino or dialkylamino head group). In principle, there are no specific limitations concerning the amino lipids of the LNP compositions disclosed herein. The cationic lipid is typically protonated (i.e., positively charged) at a pH below the pKa of the cationic lipid and is substantially neutral at a pH above the pKa. The cationic lipids can also be termed titratable cationic lipids. In some embodiments, the one or more cationic lipids include: a protonatable tertiary amine (e.g., pH-titratable) head group; alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DOTMA, DLinDMA, DLenDMA,.gamma.-DLenDMA, DLin-K-DMA, DLin-K-C2-DMA (also known as DLin-C2K-DMA, XTC2, and C2K), DLin-K-C3-DMA, DLin-K-C4-DMA, DLen-C2K-DMA, y-DLen-C2-DMA, C12-200, cKK-E12, cKK-A12, cKK-012, DLin-MC2-DMA (also known as MC2), and DLin-MC3-DMA (also known as MC3).

Anionic lipids suitable for use in lipid nanoparticles include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Neutral lipids (including both uncharged and zwitterionic lipids) suitable for use in lipid nanoparticles include, but are not limited to, diacylphosphatidylcholine, diacylphosphatidylethanolamine, ceramide, sphingomyelin, dihydrosphingomyelin, cephalin, sterols (e.g., cholesterol) and cerebrosides. In some embodiments, the lipid nanoparticle comprises cholesterol. Lipids having a variety of acyl chain groups of varying chain length and degree of saturation are available or may be isolated or synthesized by well-known techniques. Additionally, lipids having mixtures of saturated and unsaturated fatty acid chains and cyclic regions can be used. In some embodiments, the neutral lipids used in the disclosure are DOPE, DSPC, DPPC, POPC, or any related phosphatidylcholine. In some embodiments, the neutral lipid may be composed of sphingomyelin, dihydrosphingomyeline, or phospholipids with other head groups, such as serine and inositol.

In some embodiments, amphipathic lipids are included in nanoparticles. Exemplary amphipathic lipids suitable for use in nanoparticles include, but are not limited to, sphingolipids, phospholipids, fatty acids, and amino lipids.

The lipid composition of the pharmaceutical composition may comprise one or more phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular amphipathic lipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural amphipathic lipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the LNP composition comprises one or more phospholipids. In some embodiments, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16:0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine1,2-didocosahexaenoyl--sn-glycero-3-phosphoethanolamine, 1,2-dioleoyl-sn-glycero-3-phospho-rac-(1-glycerol) sodium salt (DOPG), sphingomyelin, and any mixtures thereof.

Other phosphorus-lacking compounds, such as sphingolipids, glycosphingolipid families, diacylglycerols, and.beta.-acyloxyacids, may also be used. Additionally, such amphipathic lipids can be readily mixed with other lipids, such as triglycerides and sterols.

In some embodiments, the LNP composition comprises one or more helper lipids. The term “helper lipid” as used herein refers to lipids that enhance transfection (e.g., transfection of an LNP comprising an mRNA that encodes a site-directed endonuclease, such as a SpCas9 polypeptide). In principle, there are no specific limitations concerning the helper lipids of the LNP compositions disclosed herein. Without being bound to any particular theory, it is believed that the mechanism by which the helper lipid enhances transfection includes enhancing particle stability. In some embodiments, the helper lipid enhances membrane fusogenicity. Generally, the helper lipid of the LNP compositions disclosure herein can be any helper lipid known in the art. Non-limiting examples of helper lipids suitable for the compositions and methods include steroids, sterols, and alkyl resorcinols. Particularly helper lipids suitable for use in the present disclosure include, but are not limited to, saturated phosphatidylcholine (PC) such as distearoyl-PC (DSPC) and dipalymitoyl-PC (DPPC), dioleoylphosphatidylethanolamine (DOPE), 1,2-dilinoleoyl-sn-glycero-3-phosphocholine (DLPC), cholesterol, 5-heptadecylresorcinol, and cholesterol hemisuccinate. In some embodiments, the helper lipid of the LNP composition includes cholesterol.

In some embodiments, the LNP composition comprises one or more structural lipids. As used herein, the term “structural lipid” refers to sterols and also to lipids containing sterol moieties. Without being bound to any particular theory, it is believed that the incorporation of structural lipids into the LNPs mitigates aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In some embodiments, the structural lipid is cholesterol. In certain embodiments, the structural lipid is an analog of cholesterol.

The lipid component of a lipid nanoparticle composition may include one or more molecules comprising polyethylene glycol, such as PEG or PEG-modified lipids. In some embodiments, the LNP composition disclosed herein comprise one or more polyethylene glycol (PEG) lipid. The term “PEG-lipid” refers to polyethylene glycol (PEG)-modified lipids. Such lipids are also referred to as PEGylated lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines For example, a PEG lipid can be PEG-c-DOMG, PEG-DMG, PEG-DLPE, PEG-DMPE, PEG-DPPC, or a PEG-DSPE lipid. In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the lipid moiety of the PEG-lipids includes those having lengths of from about C.sub. 14 to about C.sub. 22, preferably from about C.sub. 14 to about C.sub. 16. In some embodiments, a PEG moiety, for example a mPEG-NH.sub. 2, has a size of about 1000, 2000, 5000, 10,000, 15,000 or 20,000 daltons. In some embodiment, the PEG-lipid is PEG2k-DMG. In some embodiments, the one or more PEG lipids of the LNP composition comprises PEG-DMPE. In some embodiments, the one or more PEG lipids of the LNP composition comprises PEG-DMG.

In some embodiments, the ratio between the lipid components and the nucleic acid molecules of the LNP composition, e.g., the weight ratio, is sufficient for (i) formation of LNPs with desired characteristics, e.g., size, charge, and (ii) delivery of a sufficient dose of nucleic acid at a dose of the lipid component(s) that is tolerable for in vivo administration as readily ascertained by one of skill in the art.

In certain embodiments, it is desirable to target a nanoparticle, e.g., a lipid nanoparticle, using a targeting moiety that is specific to a cell type and/or tissue type. In some embodiments, a nanoparticle may be targeted to a particular cell, tissue, and/or organ using a targeting moiety. In particular embodiments, a nanoparticle comprises a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′) 2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42 (5): 439-62, 2003 and Abra et al., J. Liposome Res. 12:1-3, 2002.

In some embodiments, a lipid nanoparticle (e.g., a liposome) may include a surface coating of hydrophilic polymer chains, such as polyethylene glycol (PEG) chains (see, e.g., Allen et al., Biochimica et Biophysica Acta 1237:99-108, 1995; DeFrees et al., Journal of the American Chemistry Society 118:6101-6104, 1996; Blume et al., Biochimica et Biophysica Acta 1149:180-184,1993; Klibanov et al., Journal of Liposome Research 2:321-334, 1992; U.S. Pat. No. 5,013,556; Zalipsky, Bioconjugate Chemistry 4:296-299, 1993; Zalipsky, FEBS Letters 353:71-74, 1994; Zalipsky, in Stealth Liposomes Chapter 9 (Lasic and Martin, Eds) CRC Press, Boca Raton Fla., 1995). In one approach, a targeting moiety for targeting the lipid nanoparticle is linked to the polar head group of lipids forming the nanoparticle. In another approach, the targeting moiety is attached to the distal ends of the PEG chains forming the hydrophilic polymer coating (see, e.g., Klibanov et al., Journal of Liposome Research 2:321-334, 1992; Kirpotin et al., FEBS Letters 388:115-118, 1996).

Standard methods for coupling the targeting moiety or moieties may be used. For example, phosphatidylethanolamine, which can be activated for attachment of targeting moieties, or derivatized lipophilic compounds, such as lipid-derivatized bleomycin, can be used. Antibody-targeted liposomes can be constructed using, for instance, liposomes that incorporate protein A (see, e.g., Renneisen et al., J. Bio. Chem., 265:16337-16342, 1990 and Leonetti et al., Proc. Natl. Acad. Sci. (USA), 87:2448-2451, 1990). Other examples of antibody conjugation are disclosed in U.S. Pat. No. 6,027,726. Examples of targeting moieties can also include other polypeptides that are specific to cellular components, including antigens associated with neoplasms or tumors. Polypeptides used as targeting moieties can be attached to the liposomes via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some embodiments, a lipid nanoparticle includes a targeting moiety that targets the lipid nanoparticle to a cell including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte.

The lipid nanoparticles described herein may be lipidoid-based. The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat. Biotechnol. 2008 26:561-569; Love et al., Proc Natl Acad Sci USA. 2010 107:1864-1869; Siegwart et al., Proc Natl Acad Sci USA. 2011 108:12996-3001).

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see e.g., Akinc et al., Mol Ther. 2009 17:872-879), use of lipidoid oligonucleotides to deliver the formulation to other cells types including, but not limited to, endothelial cells, myeloid cells, and muscle cells may not be similarly size-limited.

In one aspect, effective delivery to myeloid cells, such as monocytes, lipidoid formulations may have a similar component molar ratio. Different ratios of lipidoids and other components including, but not limited to, a neutral lipid (e.g., diacylphosphatidylcholine), cholesterol, a PEGylated lipid (e.g., PEG-DMPE), and a fatty acid (e.g., an omega-3 fatty acid) may be used to optimize the formulation of the mRNA or system for delivery to different cell types including, but not limited to, hepatocytes, myeloid cells, muscle cells, etc. Exemplary lipidoids include, but are not limited to, DLin-DMA, DLin-K-DMA, DLin-KC2-DMA, 98N12-5, C12-200 (including variants and derivatives), DLin-MC3-DMA and analogs thereof. The use of lipidoid formulations for the localized delivery of nucleic acids to cells (such as, but not limited to, adipose cells and muscle cells) via either subcutaneous or intramuscular delivery, may also not require all of the formulation components which may be required for systemic delivery, and as such may comprise the lipidoid and the mRNA or system.

According to the present disclosure, a system described herein may be formulated by mixing the mRNA or system, or individual components of the system, with the lipidoid at a set ratio prior to addition to cells. In vivo formulations may require the addition of extra ingredients to facilitate circulation throughout the body. After formation of the particle, a system or individual components of a system is added and allowed to integrate with the complex. The encapsulation efficiency is determined using a standard dye exclusion assays.

In vivo delivery of systems may be affected by many parameters, including, but not limited to, the formulation composition, nature of particle PEGylation, degree of loading, oligonucleotide to lipid ratio, and biophysical parameters such as particle size (Akinc et al., Mol Ther. 2009 17:872-879; herein incorporated by reference in its entirety). As an example, small changes in the anchor chain length of poly (ethylene glycol) (PEG) lipids may result in significant effects on in vivo efficacy. Formulations with the different lipidoids, including, but not limited to penta [3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), MD1, DLin-DMA, DLin-K-DMA, DLin-KC2-DMA and DLin-MC3-DMA can be tested for in vivo activity. The lipidoid referred to herein as “98N12-5” is disclosed by Akinc et al., Mol Ther. 2009 17:872-879). The lipidoid referred to herein as “C12-200” is disclosed by Love et al., Proc Natl Acad Sci USA. 2010 107:1864-1869 and Liu and Huang, Molecular Therapy. 2010 669-670.

LNPs in which a nucleic acid is entrapped within the lipid portion of the particle and is protected from degradation, can be formed by any method known in the art including, but not limited to, a continuous mixing method, a direct dilution process, and an in-line dilution process. Additional techniques and methods suitable for the preparation of the LNPs described herein include coacervation, microemulsions, supercritical fluid technologies, phase-inversion temperature (PIT) techniques.

In some embodiments, the LNPs used herein are produced via a continuous mixing method, e.g., a process that includes providing an aqueous solution a nucleic acid described herein in a first reservoir, providing an organic lipid solution in a second reservoir (wherein the lipids present in the organic lipid solution are solubilized in an organic solvent, e.g., a lower alkanol such as ethanol), and mixing the aqueous solution with the organic lipid solution such that the organic lipid solution mixes with the aqueous solution so as to substantially instantaneously produce a lipid vesicle (e.g., liposome) encapsulating the nucleic acid molecule within the lipid vesicle. This process and the apparatus for carrying out this process are known in the art. More information in this regard can be found in, for example, U.S. Patent Publication No. 20040142025. The action of continuously introducing lipid and buffer solutions into a mixing environment, such as in a mixing chamber, causes a continuous dilution of the lipid solution with the buffer solution, thereby producing a lipid vesicle substantially instantaneously upon mixing. By mixing the aqueous solution comprising a nucleic acid molecule with the organic lipid solution, the organic lipid solution undergoes a continuous stepwise dilution in the presence of the buffer solution (e.g., aqueous solution) to produce a nucleic acid-lipid particle.

In some embodiments, the LNPs used herein are produced via a direct dilution process that includes forming a lipid vesicle (e.g., liposome) solution and immediately and directly introducing the lipid vesicle solution into a collection vessel containing a controlled amount of dilution buffer. In some embodiments, the collection vessel includes one or more elements configured to stir the contents of the collection vessel to facilitate dilution. In some embodiments, the amount of dilution buffer present in the collection vessel is substantially equal to the volume of lipid vesicle solution introduced thereto.

In some embodiments, the LNPs are produced via an in-line dilution process in which a third reservoir containing dilution buffer is fluidly coupled to a second mixing region. In these embodiments, the lipid vesicle (e.g., liposome) solution formed in a first mixing region is immediately and directly mixed with dilution buffer in the second mixing region. These processes and the apparatuses for carrying out direct dilution and in-line dilution processes are known in the art. More information in this regard can be found in, for example, U.S. Patent Publication No. 20070042031.

### 6.12.2. Viral Vector Delivery

In certain aspects the invention involves vectors, e.g. for delivering or introducing in a cell, but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

Vector delivery, e.g., plasmid, viral delivery: The CRISPR enzyme, for instance a Type V protein such as C2cl or C2c3, and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. Effector proteins and one or more guide RNAs can be packaged into one or more vectors, e.g., plasmid or viral vectors. In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹¹ particles), more preferably at least about 1×10⁷ particles, more preferably at least about 1×10⁸ particles (e.g., about 1×10⁸-1×10¹¹ particles or about 1×10⁹-1×10¹² particles), and most preferably at least about 1×10¹⁰ particles (e.g., about 1×10⁹-1×10¹⁰ particles or about 1×10⁹-1×10¹² particles), or even at least about 1 ×10¹⁰ particles (e.g., about 1×10¹⁰-1×10¹² particles) of the adenoviral vector. Alternatively, the dose comprises no more than about 1×10¹⁴ particles, preferably no more than about 1×1013 particles, even more preferably no more than about 1×10¹² particles, even more preferably no more than about 1×10¹¹ particles, and most preferably no more than about 1×10¹⁰ particles (e.g., no more than about 1×10⁹ particles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et, al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

The promoter used to drive nucleic acid-targeting effector protein coding nucleic acid molecule expression can include: AAV ITR can serve as a promoter: this is advantageous for eliminating the need for an additional promoter element (which can take up space in the vector). The additional space freed up can be used to drive the expression of additional elements (gRNA, etc.). Also, ITR activity is relatively weaker, so can be used to reduce potential toxicity due to over expression of nucleic acid-targeting effector protein. For ubiquitous expression, can use promoters: CMV, CAG, CBh, PGK, SV40, Ferritin heavy or light chains, etc. For brain or other CNS expression, can use promoters: SynapsinI for all neurons, CaMKIIalpha for excitatory neurons, GAD67 or GAD65 or VGAT for GABAergic neurons, etc. For liver expression, can use Albumin promoter. For lung expression, can use SP-B. For endothelial cells, can use ICAM. For hematopoietic cells can use IFNbeta or CD45. For Osteoblasts can use OG-2.

The promoter used to drive guide RNA can include: Pol III promoters such as U6 or H1 Use of Pol II promoter and intronic cassettes to express guide RNA Adeno Associated Virus (AAV)

Nucleic acid-targeting effector protein and one or more guide RNA can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific genome modification, the expression of nucleic acid-targeting effector can be driven by a cell-type specific promoter. For example, liver-specific expression might use the Albumin promoter and neuron-specific expression (e.g., for targeting CNS disorders) might use the Synapsin I promoter.

In terms of in vivo delivery, AAV is advantageous over other viral vectors for a couple of reasons: Low toxicity (this may be due to the purification method not requiring ultra centrifugation of cell particles that can activate the immune response) and Low probability of causing insertional mutagenesis because it doesn't integrate into the host genome.

AAV has a packaging limit of 4.5 or 4.75 Kb. This means that nucleic acid-targeting effector protein (such as a Type V protein such as C2cl or C2c3) as well as a promoter and transcription terminator have to be all fit into the same viral vector. Therefore embodiments of the invention include utilizing homologs of nucleic acid-targeting effector protein (such as a Type V protein such as C2cl or C2c3) that are shorter.

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The herein promoters and vectors are preferred individually.

Packaging cells are typically used to form virus particles that are capable of infecting a host cell. Such cells include 293 cells, which package adenovirus, and psi2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

Millington-Ward et al. (Molecular Therapy, vol. 19 no. 4, 642-649 Apr. 2011) describes adeno-associated virus (AAV) vectors to deliver an RNA interference (RNAi)-based rhodopsin suppressor and a codon-modified rhodopsin replacement gene resistant to suppression due to nucleotide alterations at degenerate positions over the RNAi target site. An injection of either 6.0×10⁸ vp or 1.8×10¹⁰ vp AAV were subretinally injected into the eyes by Millington-Ward et al. The AAV vectors of Millington-Ward et al. may be applied to the system of the present invention, contemplating a dose of about 2×10¹¹ to about 6×10¹¹ vp administered to a human.

Dalkara et al. (Sci Transl Med 5, 189ra76 (2013)) also relates to in vivo directed evolution to fashion an AAV vector that delivers wild-type versions of defective genes throughout the retina after noninjurious injection into the eyes' vitreous humor. Dalkara describes a 7 mer peptide display library and an AAV library constructed by DNA shuffling of cap genes from AAV1, 2, 4, 5, 6, 8, and 9. The rcAAV libraries and rAAV vectors expressing GFP under a CAG or Rho promoter were packaged and deoxyribonuclease-resistant genomic titers were obtained through quantitative PCR. The libraries were pooled, and two rounds of evolution were performed, each consisting of initial library diversification followed by three in vivo selection steps. In each such step, P30 rho-GFP mice were intravitreally injected with 2 ml of iodixanol-purified, phosphate-buffered saline (PBS)-dialyzed library with a genomic titer of about 1.times. 10.sup. 12 vg/ml. The AAV vectors of Dalkara et al. may be applied to the nucleic acid-targeting system of the present invention, contemplating a dose of about 1×10¹⁵ to about 1×10¹⁶ vg/ml administered to a human.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SW), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and yr2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. Cells taken from a subject include, but are not limited to, hepatocytes or cells isolated from muscle, the CNS, eye or lung. Immunological cells are also contemplated, such as but not limited to T cells, HSCs, B-cells and NK cells.

Another useful method to deliver proteins, enzymes, and guides comprises transfection of messenger RNA (mRNA). Examples of mRNA delivery methods and compositions that may be utilized in the present disclosure including, for example, PCT/US2014/028330, U.S. Pat. No. 8,822,663B2, NZ700688A, ES2740248T3, EP2755693 A4, EP2755986A4, WO2014152940A1, EP3450553B1, BR112016030852A2, and EP3362461A1. Expression of CRISPR systems in particular is described by WO2020014577. Each of these publications are incorporated herein by reference in their entireties. Additional disclosure hereby incorporated by reference can be found in Kowalski et al., “Delivering the Messenger: Advances in Technologies for Therapeutic mRNA Delivery,” Mol Therap., 2019; 27 (4): 710-728.

In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CVI, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd. 3, BHK-21, BR 293, BxPC3, C3H-10T1/2, C6/36, Cal-27, CHO, CHO-7, CHO—IR, CHO-K1, CHO-K2, CHO-T, CHO Dhfr-/-, COR-L23, COR-L23/CPR, COR-L23/5010, COR-L23/R23, COS-7, COV-434, CML TI, CMT, CT26, D17, DH82, DU145, DuCaP, EL4, EM2, EM3, EMT6/ARI, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THPI cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences.

In some embodiments, one or more vectors described herein are used to produce a non-human transgenic animal or transgenic plant. In some embodiments, the transgenic animal is a mammal, such as a mouse, rat, or rabbit. In certain embodiments, the organism or subject is a plant. In certain embodiments, the organism or subject or plant is algae. Methods for producing transgenic plants and animals are known in the art, and generally begin with a method of cell transfection, such as described herein.

In one aspect, the invention provides for methods of modifying a target polynucleotide in a prokaryotic or eukaryotic cell, which may be in vivo, ex vivo or in vitro. In some embodiments, the method comprises sampling a cell or population of cells from a human or non-human animal or plant (including micro-algae) and modifying the cell or cells. Culturing may occur at any stage ex vivo. The cell or cells may even be re-introduced into the non-human animal or plant (including micro-algae).

In plants, pathogens are often host-specific. For example, Fusariumn oxysporum f. sp. lycopersici causes tomato wilt but attacks only tomato, and F. oxysporum f. dianthii Puccinia graminis f. sp. tritici attacks only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield. Quality, Uniformity, Hardiness, Resistance. The sources of resistance genes include native or foreign Varieties, Heirloom Varieties, Wild Plant Relatives, and Induced Mutations, e.g., treating plant material with mutagenic agents. Using the present invention, plant breeders are provided with a new tool to induce mutations. Accordingly, one skilled in the art can analyze the genome of sources of resistance genes, and in Varieties having desired characteristics or traits employ the present invention to induce the rise of resistance genes, with more precision than previous mutagenic agents and hence accelerate and improve plant breeding programs.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown and may be at a normal or abnormal level.

### 7. EXAMPLES

### 7.1. Example 1: Single Nucleic Acid Construct Comprising PASTE Components and a Nucleic Acid Cargo of Interest that is Capable of Recombinase-Mediated Subsequence Circularization Effects Targeted Integration of the Cargo into a Genomic Locus

A single construct “installer” that contains a prime editor fusion protein, an attachment site-containing guide RNA (atgRNA), a nickase guide RNA (ngRNA), an integrase, a recombinase, recombination target sites, integration target site, a DNA of interest, and flanking ITRs is designed (FIG. 1). Following delivery of the single nucleic acid construct “installer”, recombinase expression and binding at recombinase recognition sites leads to self-circularization of a subsequence of the single nucleic acid construct. A DNA of interest (e.g. gene) contained within the self-circularized nucleic acid integrates into a genomic locus of interest via an integrase. Genomic integration occurs at an integrase recognition target site (i.e., “beacon”) placed via prime editing or gene writing. For additional disclosure regarding the nucleic acid construct, self-circularization and integration see, for example, Section 6.9 and 6.10.

### 7.2. Example 2: Single Nucleic Acid Construct Comprising PASTE Components and a Nucleic Acid Cargo of Interest that is Capable of Integrase-Mediated Subsequence Circularization Effects Targeted Integration of the Cargo into a Genomic Locus

A single construct “installer” that contains a prime editor fusion protein, an attachment site-containing guide RNA (atgRNA), a nickase guide RNA (ngRNA), an integrase, integration target sites, a DNA of interest, and flanking ITRs is designed (FIG. 2). Following delivery of the single nucleic acid construct “installer”, integrase expression and binding at integrase recognition sites (attP2/attB2) leads to self-circularization of a subsequence of the single nucleic acid construct.

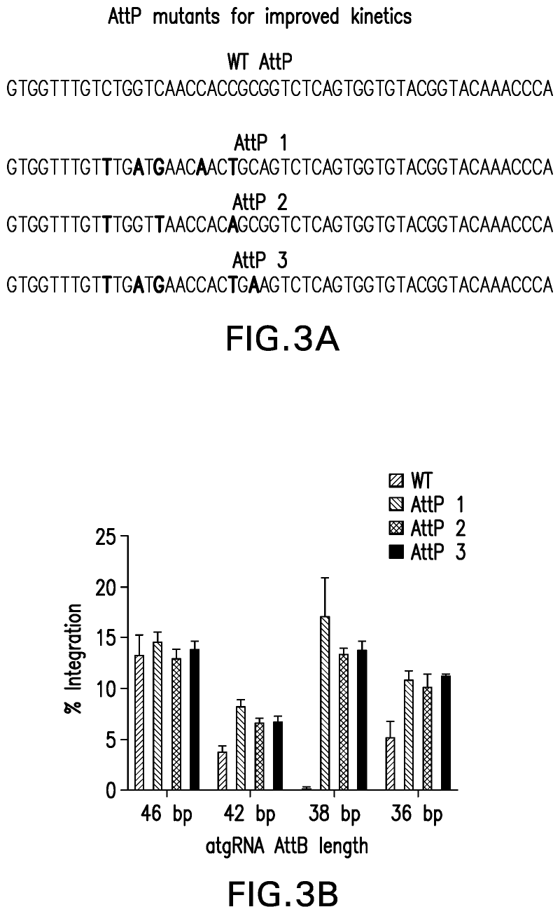

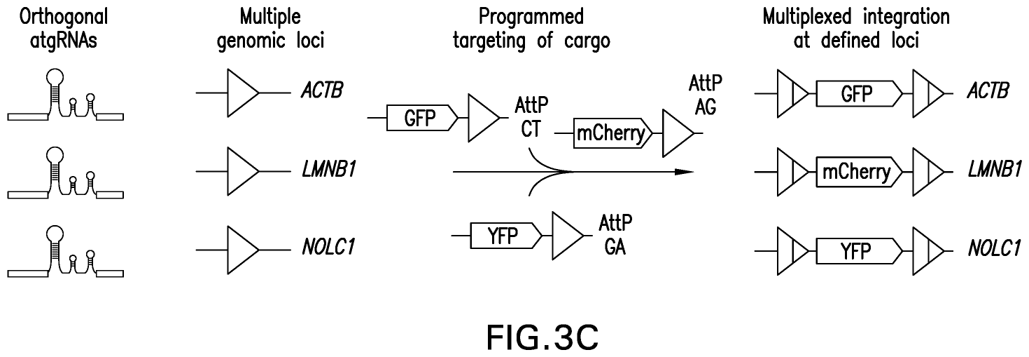

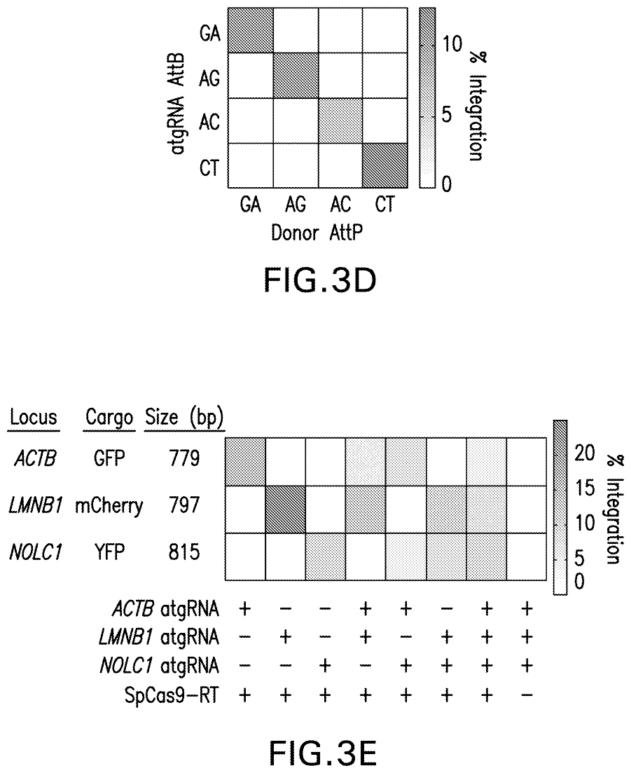

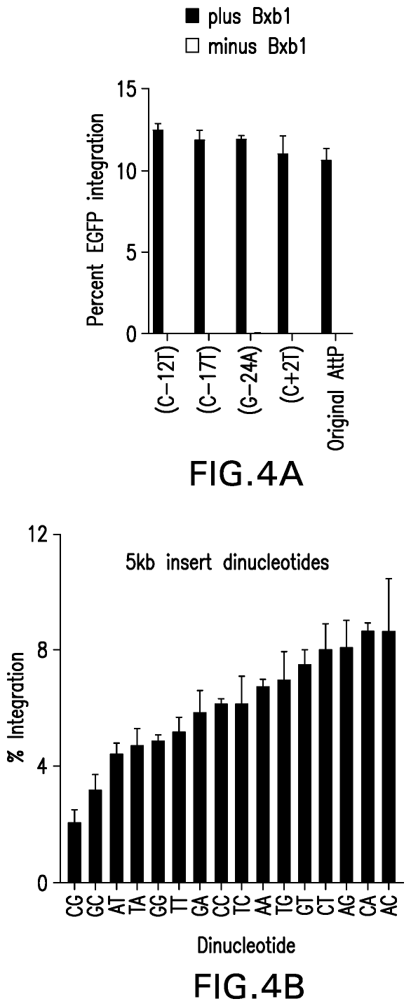

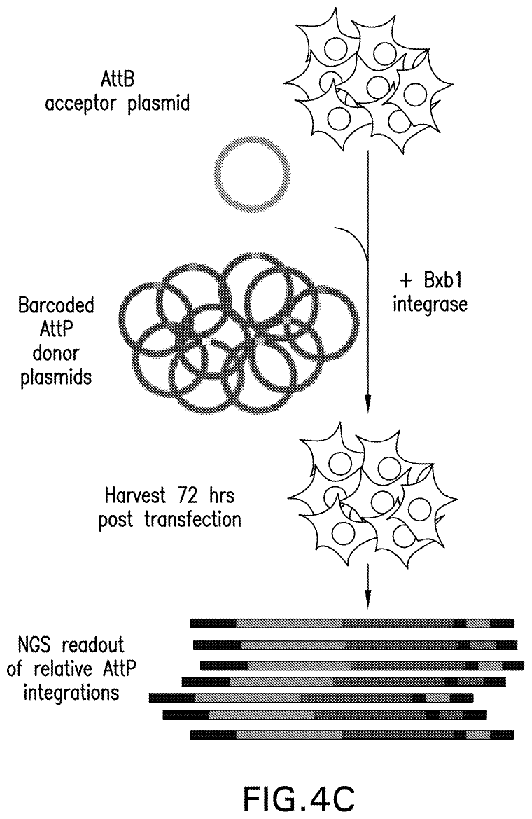

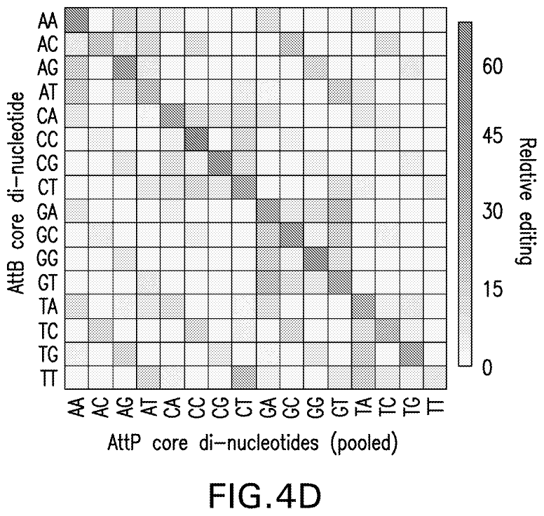

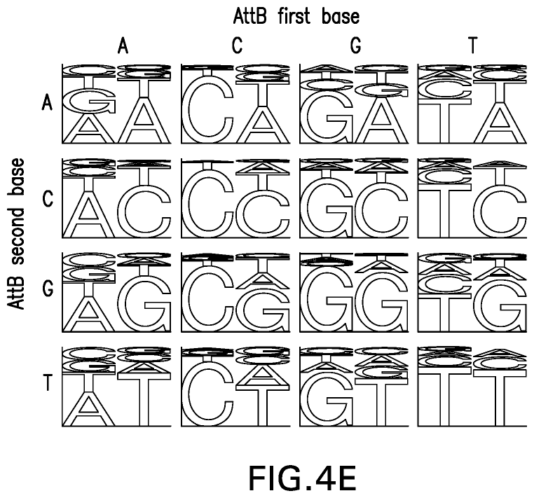

Stepwise control of self-circularization followed by genomic integration is achieved by use of central dinucleotide matched orthogonal integrase target recognition sites (i.e., attB/attP pairs) (FIG. 3D and FIG. 4D). Additionally, use of a kinetically fast attB/attP pair integrated into the single nucleic acid construct allows self-circularization prior to genomic integration. Screening of attB/attP pairs is achieved through a pooled attB/attP dinucleotide orthogonality assay (FIG. 4C) and relative insertion preferences for all attB/attP dinucleotide pairs results shown in FIG. 4E. Improved genomic integration occurs via the selection of attP/attB mutant pairs (FIG. 3A) that demonstrate improved integration efficiency (FIGS. 3B-C and FIGS. 4A-4B).

A DNA of interest (e.g., gene) contained within the self-circularized nucleic acid integrates into a genomic locus of interest via the integrase via the attP1/attB1 sites. Genomic integration occurs at an attB1 integrase recognition target site (i.e., “beacon”) placed via prime editing or gene writing.

### 7.3. Example 3: Single Nucleic Acid Construct Comprising PASTE Components Wherein an Integrase is Linked to a Prime Editor and a Nucleic Acid Cargo of Interest that is Capable of Integrase-Mediated Subsequence Circularization Effects Targeted Integration of the Cargo into a Genomic Locus

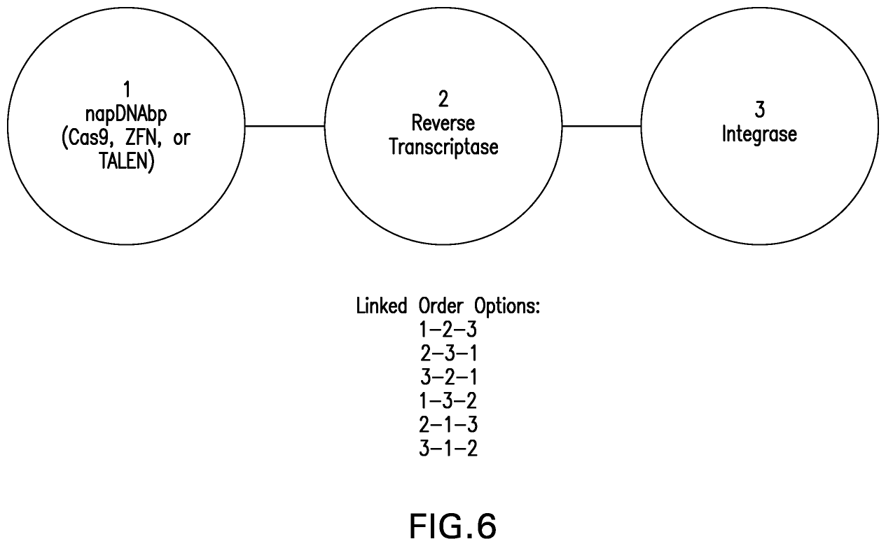

A single construct “installer” that contains a prime editor fusion protein linked to an integrase (FIG. 6), an attachment site-containing guide RNA (atgRNA), a nickase guide RNA (ngRNA), an integrase, integration target sites, a DNA of interest, and flanking ITRs is designed. Following delivery of the single nucleic acid construct “installer”, prime editor-integrase fusion (Cas9-RT-Integrase) expression and binding at integrase recognition sites (attP2/attB2) leads to self-circularization of a subsequence of the single nucleic acid construct.

Stepwise control of self-circularization followed by genomic integration is achieved by use of central dinucleotide matched orthogonal integrase target recognition sites (i.e., attB/attP pairs) (FIG. 3D and FIG. 4D). Additionally, use of a kinetically fast attB/attP pair integrated into the single nucleic acid construct allows self-circularization prior to genomic integration. Screening of attB/attP pairs is achieved through a pooled attB/attP dinucleotide orthogonality assay (FIG. 4C) and relative insertion preferences for all attB/attP dinucleotide pairs results shown in FIG. 4E. Improved genomic integration occurs via the selection of attP/attB mutant pairs (FIG. 3A) that demonstrate improved integration efficiency (FIG. 3B and FIG. 4B).

A DNA of interest (e.g., gene) contained within the self-circularized nucleic acid integrates into a genomic locus of interest via the integrase via the attP1/attB1 sites. Genomic integration occurs at an attB1 integrase recognition target site (i.e., “beacon”) placed via prime editing mediated by the prime editor-integrase fusion.

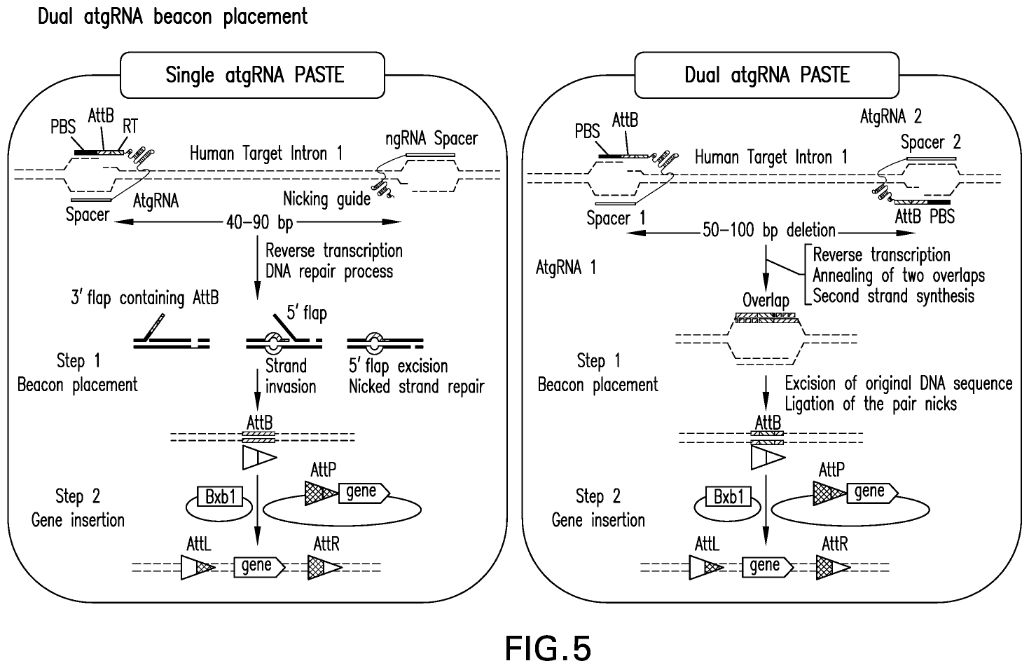

FIG. 5 illustrates a schematic of single atgRNA and dual atgRNA approaches for beacon placement. The single construct “installer” that contains a prime editor fusion protein linked to an integrase (FIG. 6), a first attachment site-containing guide RNA (atgRNA), a second attachment site-containing guide (atgRNA), an integrase, integration target sites, a DNA of interest, and flanking ITRs is designed. In this version of the single construct “installer” the first atgRNA and the second atgRNAs collectively encode the entirety of the integration recognition site.

### 7.4. Example 4: Extrachromosomal Circular DNA (EccDNA) Sensor to Evaluate Template Integrase-Mediated Circularization and Programmable Gene Insertion within a ACTB Beacon Locus

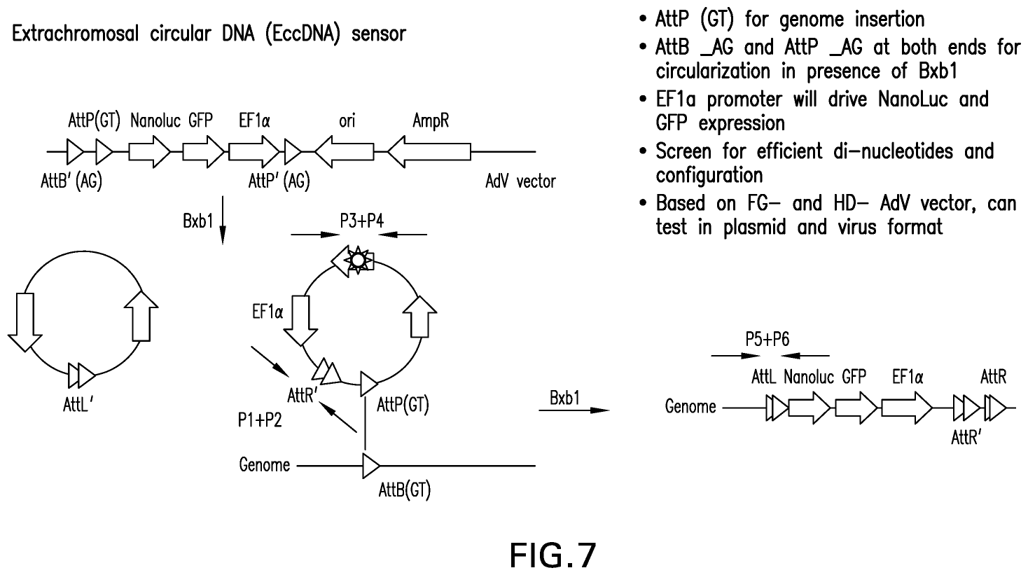

A dual reporter (Nanoluc and GFP) extrachromosomal circular DNA (EccDNA) sensor capable of detecting Bxb1-mediated self-circularization was designed (FIG. 7). B×B1-mediated circularization of the EccDNA sensor, which occurs at a attP′/attB′ target recognition site within the EccDNA sensor, orients the EF1a promoter upstream of nanoluc and GFP, thereby allowing for dual reporter expression. EccDNA circularization can also be confirmed by PCR amplification of the post-circularization attR′ scar using primers PI and P2 as shown in FIG. 7. Total EccDNA (linear and circularized) is quantified by primers P3 and P4 as shown in FIG. 7. The EccDNA construct contains an orthogonal attP (GT central dinucleotide, see FIGS. 4A and 4D) to facilitate genomic insertion at a placed attB beacon site. Genomic integration of the EccDNA is verified using primers P5 and P6 (FIG. 7).

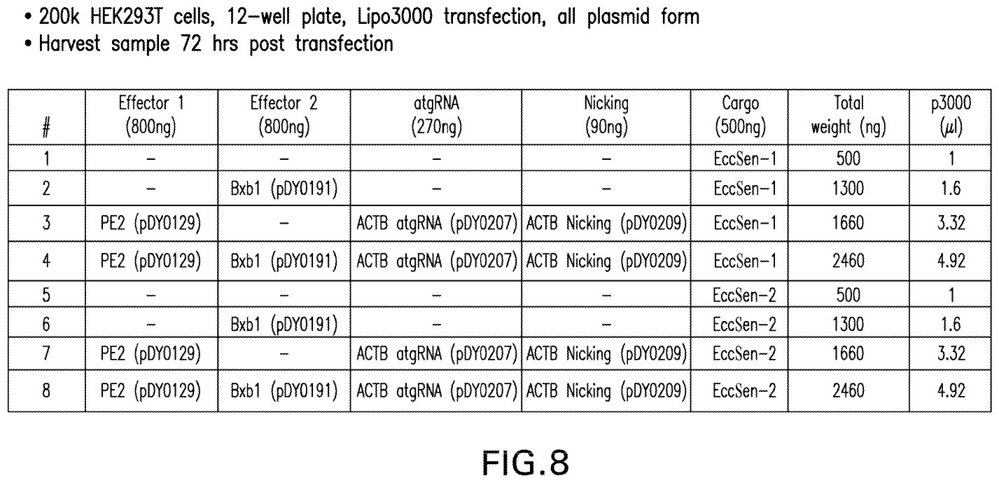

A transfection screen was performed to confirm Bxb1-mediated EccDNA circularization (FIG. 8). Plasmid expressed EccDNA sensor, prime editor protein, Bxb1, ACTB targeting atgRNA, and nicking guide RNA were transfected using Lipo3000 into HEK293T cells (200K cells in a 12-well plate). Cell samples were harvested 72 hours post transfection for circularization, beacon placement, and insertion analysis.

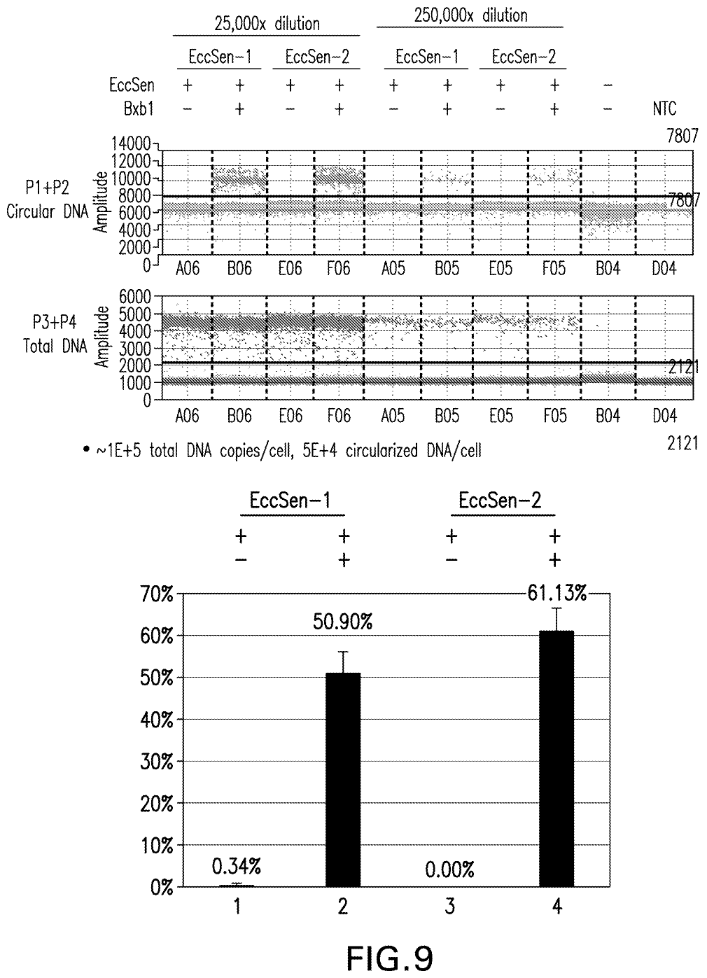

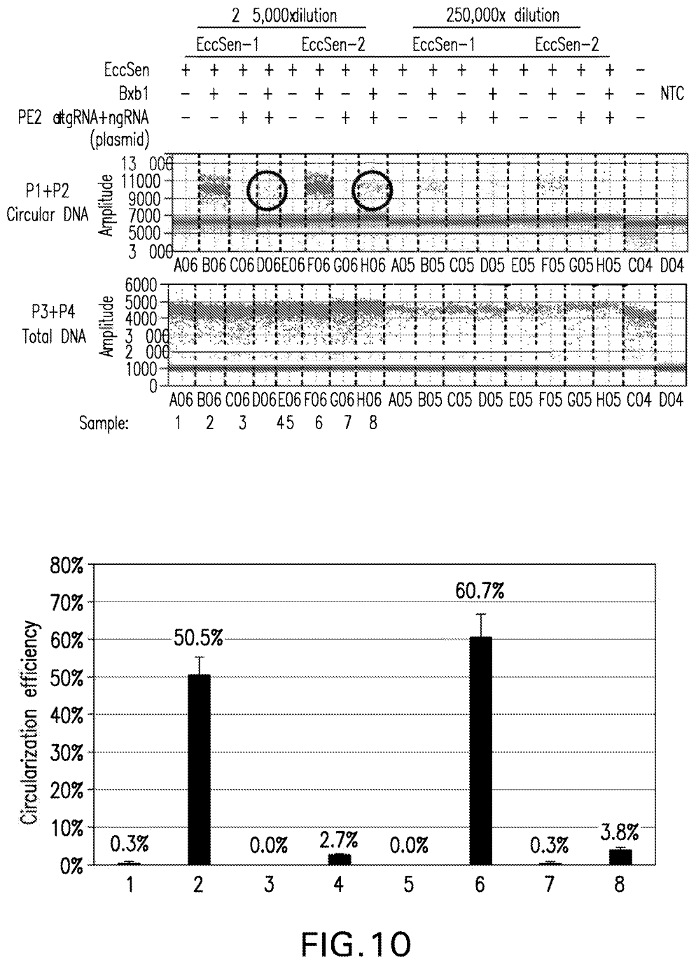

As confirmed by ddPCR, transfection of both EccDNA sensor and Bxb 1 resulted in confirmed intracellular circularization (FIG. 9). Circularization efficiency was >50% for Bxb1-containing samples tested at a 25,000-fold dilution, whereas equivalent samples that lacked B×B1 demonstrated <1% circularization. In addition to B×B1 transfection, circularization occurred with plasmid-form transfection of PE2 prime editor, A (TB targeting atgRNA, and nicking guide RNA (FIG. 10), albeit at <4% circularization efficiency. It is hypothesized that the drop in circularization efficiency is due an interaction between the plasmid-form atgRNA attB and the EccDNA AttP in the presence of B×B1. Unwanted cross talk is mitigated by use of synthetic RNAs that contain stabilizing chemical modifications.

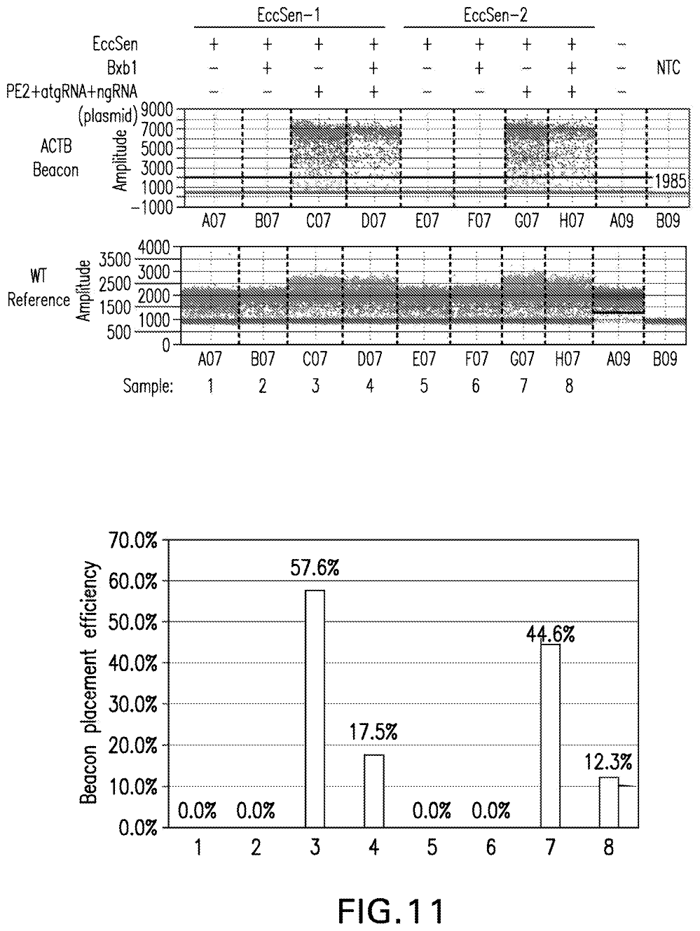

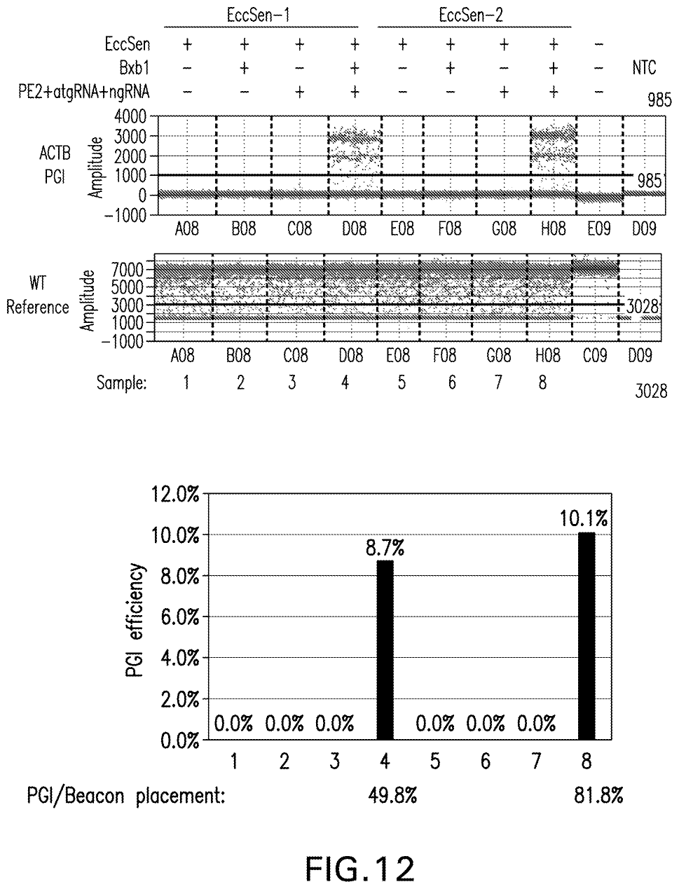

Beacon placement facilitated by the plasmid-form transfection of PE2 prime editor, A (TB targeting atgRNA, and nicking guide RNA was verified by ddPCR (FIG. 11). Beacon placement efficiency was >40% for samples containing the requisite beacon placement PE2/atgRNA/ngRNA components, however samples that also included Bxb1 demonstrated <20% beacon placement. It is hypothesized that the drop in beacon placement efficiency is due an interaction between the plasmid-form atgRNA attB and the EccDNA AttP in the presence of B×B1. FIG. 12 demonstrates programmable gene insertion of the EccDNA at the A (TB beacon locus was confirmed by ddPCR.

### 7.5. Example 5: Extrachromosomal Circular DNA (EccDNA) Sensor to Evaluate Template Integrase-Mediated Circularization and Programmable Gene Insertion within a LMNB Placed Beacon

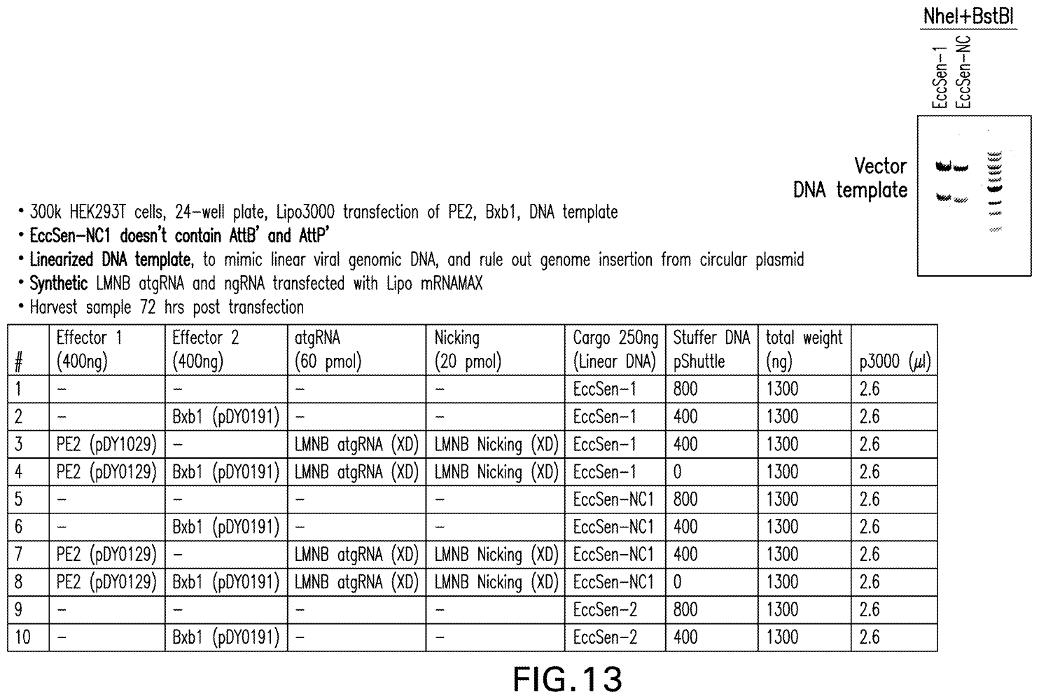

A transfection screen was performed to confirm Bxb1-mediated EccDNA circularization and subsequent programmable gene insertion at a LMNB placed attB beacon site. To mimic linear viral genomic DNA and to eliminate the potential for unwanted genome insertion of a transfected plasmid directly, a linearized EccDNA sensor was tested in cell transfections (FIG. 13). An EccDNA sensor called EccDNA-NC1 which lacks the attP′/B′ cognate pair was developed as a non-circularizing negative control. LMNB targeting atgRNA and nicking guide RNA were transfected as synthetic RNAs (containing standard IDT chemical modifications). Prime editor protein and Bxb1 effectors were transfected in plasmid form. Transfection was conducted across 300,000 HEK293T cells in a 24-well plate format using Lipo3000 for plasmid delivery (PE2, B×B1, and EccDNA sensors) in conjunction with Lipo mRNAMAX for synthetic RNA delivery (atgRNA, ngRNA). Cell samples were harvested 72 hours post transfection for circularization, beacon placement, and insertion analysis.

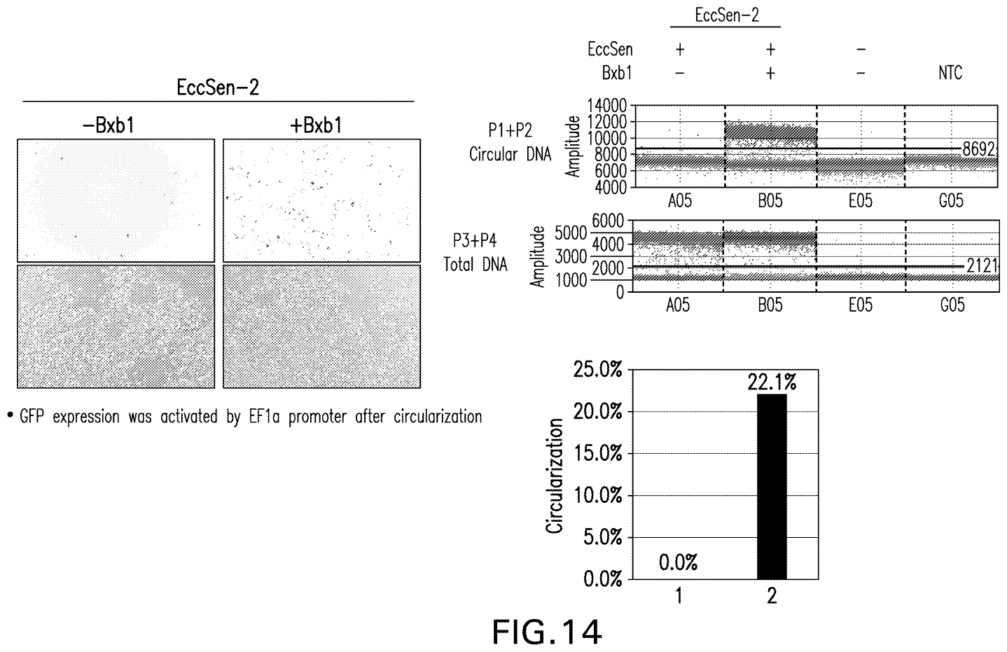

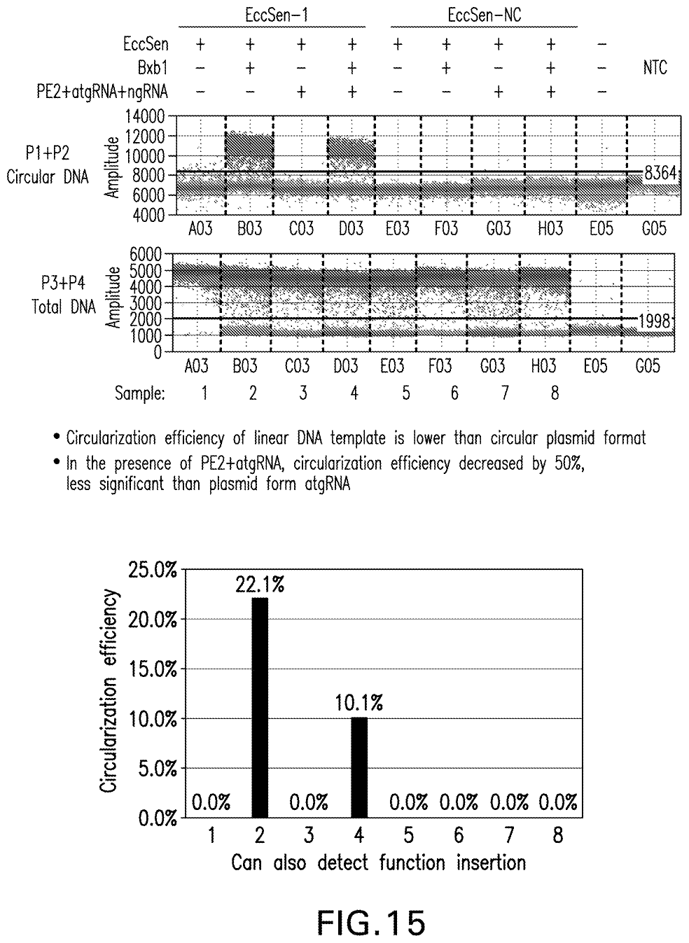

Intracellular circularization of the EccDNA sensor in the presence of B×BI was confirmed via GFP expression (FIG. 14). In a ddPCR format, co-delivery of EccDNA with B×B1 also demonstrated circularization (FIG. 15), whereas no circularization was observed in either the no B×BI control or across any of the EccDNA-NCI control replicates. EccDNA circularization was observed in the presence of B×Bland PE2/atgRNA/ngRNA (FIG. 15).

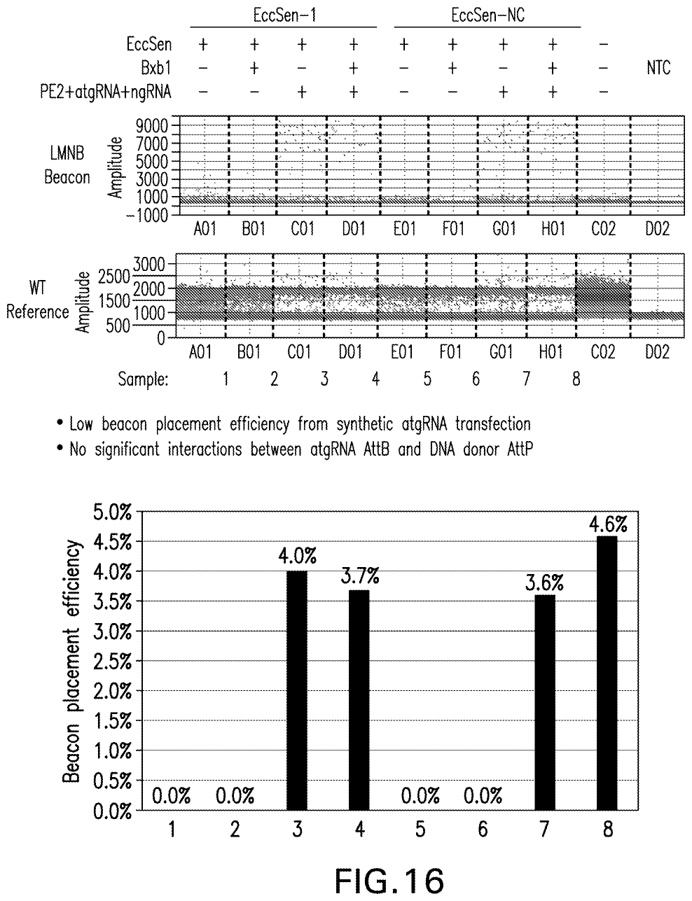

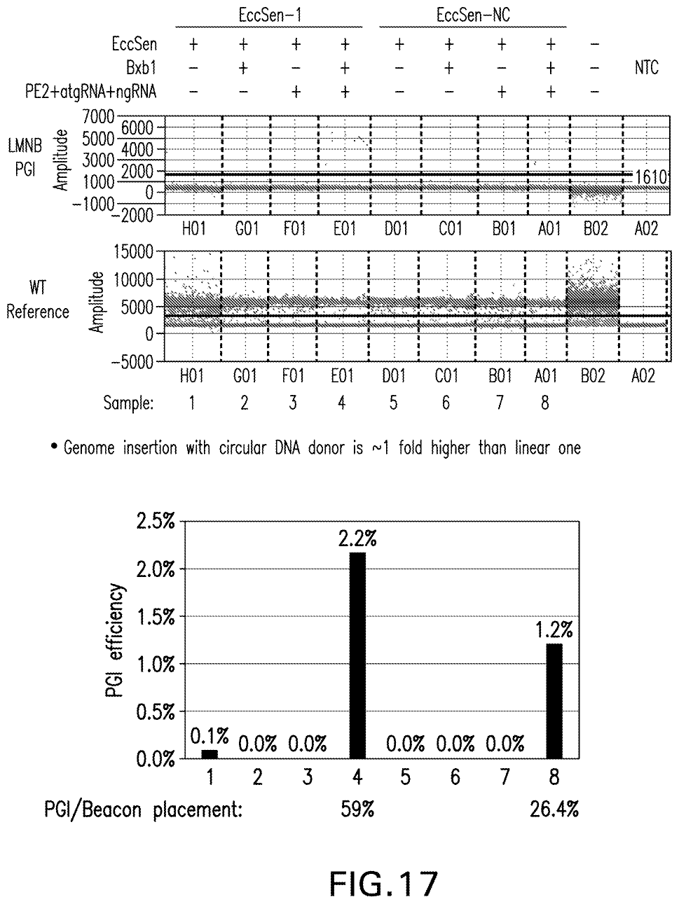

Transfection of PE2 (plasmid form) with atgRNA/ngRNA (synthetic RNA form) did result in LMNB beacon placement, however at <5% beacon placement efficiency, with a further drop in efficiency observed when Bxb1 is co transfected (FIG. 16). Low (˜1-2%) PGI of the linear EccDNA was observed Co-at the LMNB placed beacon (FIG. 17).

### 7.6. Example 6: Programmable Gene Insertion with a Single Nucleic Acid Construct (HDAd) in Mouse Cells

In this example, a single nucleic acid construct having PGI components “all-in-one” (i.e., nucleotide sequence encoding the prime editor fusion protein, nucleotide sequence encoding a first atgRNA, a nucleotide sequence encoding a second atgRNA, a nucleotide sequence encoding an integrase, and a nucleic acid cargo) was compared with a four plasmid system to see which resulted in greater beacon placement, PGI, and PGI conversion rate.

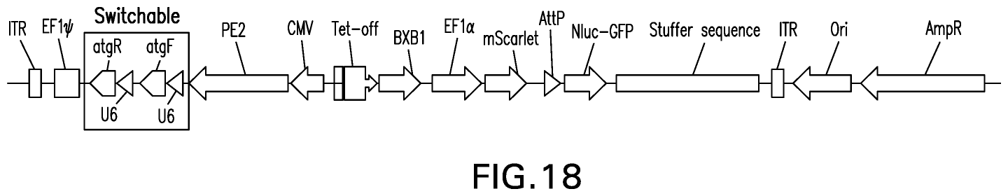

An “all-in-one” construct as shown in FIG. 18 was cloned in an adenoviral backbone (a helper dependent Adenoviral backbone) (SEQ ID NO: 559) using multistep Gibson assembly. Two clones (i.e., C5 and C8) were selected and used for further analysis. For the four plasmid system, the same components as shown in FIG. 18 were cloned into four separate plasmids (e.g., a plasmid with a nucleotide sequence encoding a prime editor fusion protein and a nucleotide sequence encoding an integrase, a second plasmid encoding a first atgRNA, a third plasmid encoding a second atgRNA, and a fourth plasmid having the nucleic acid cargo.

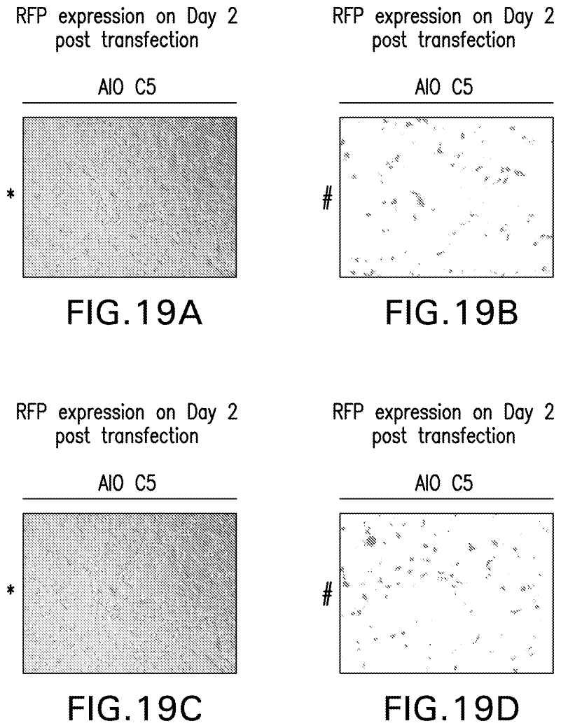

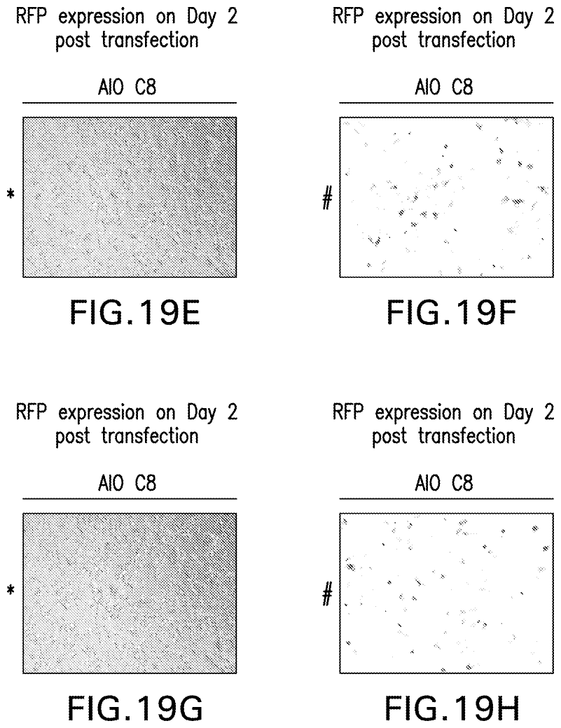

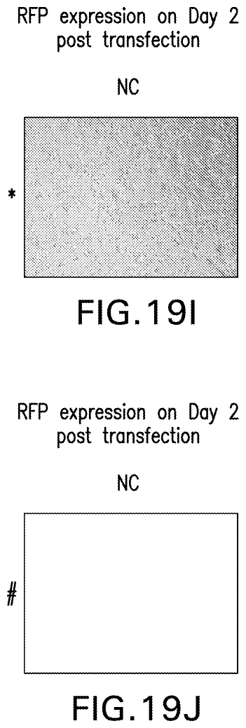

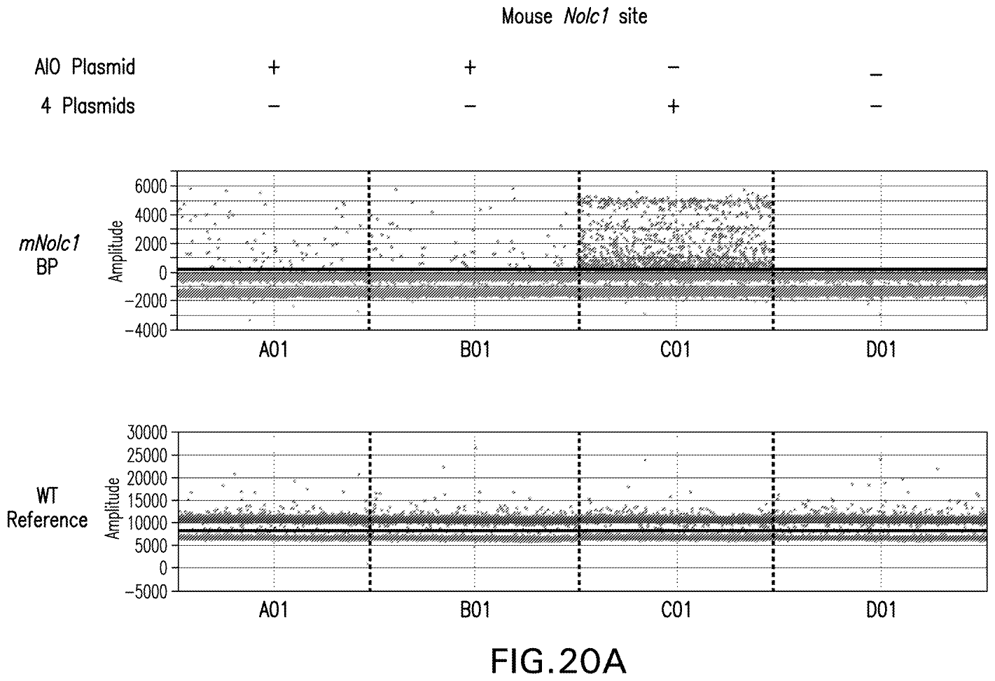

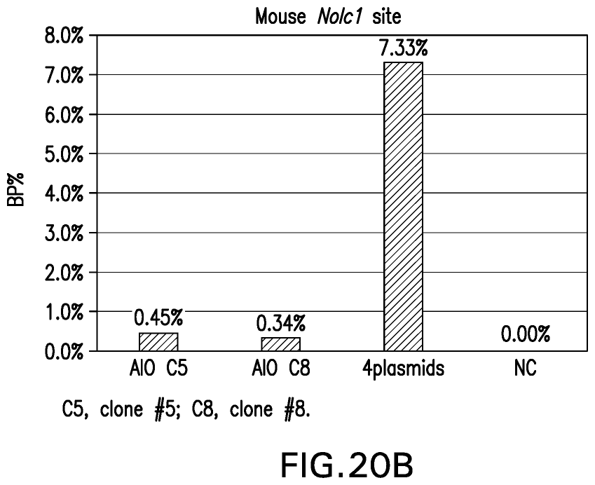

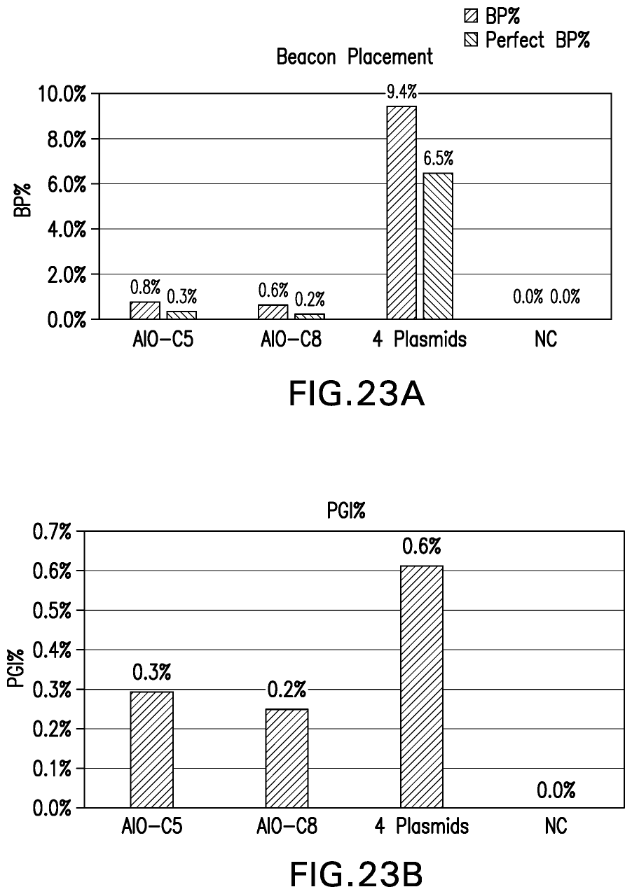

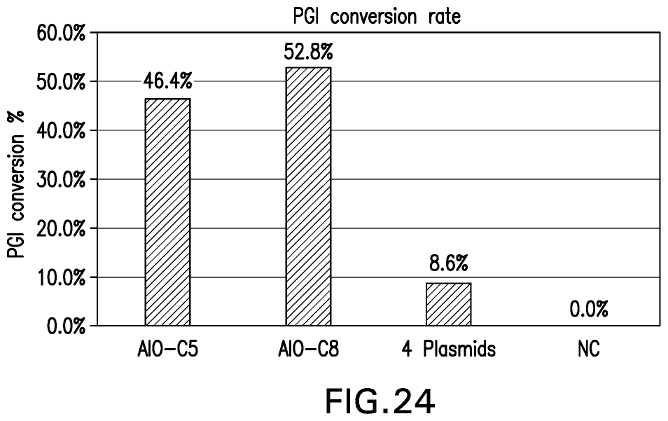

Mouse Hepa 1-6 cells were transfected in a 48 well format with 50,000 cells per well seeded 1 day prior to transfection. Total of 200 ng plasmid DNA was transfected in each well using Lipfectamine 3000 (ThermoFisher) using 3:1 (Lipo3000: DNA). As shown in FIG. 18, RFP driven by an EF1alpha promoter was used a marker for transduction. FIGS. 19A-19J shows successful transduction for both clones with RFP positive cells at day 2 post transfection. 72 hours after transfection RNA was collected and subjected to ddPCR and NGS analysis to assess beacon placement and PGI. Data for ddPCR is shown in FIGS. 20A-20B, FIGS. 21A-21B, and FIG. 22. NGS data is shown in in FIGS. 23A-23B and FIG. 24.

Beacon placement at the Nolc1 site in mouse Hepa 1-6 cells was detected using ddPCR (FIG. 20A and FIG. 20B). In particular, transfection of both single nucleic acid constructs (both clones) resulted in beacon placement at the Nolc1 site but was lower than when PGI components were delivered using a four plasmid system.

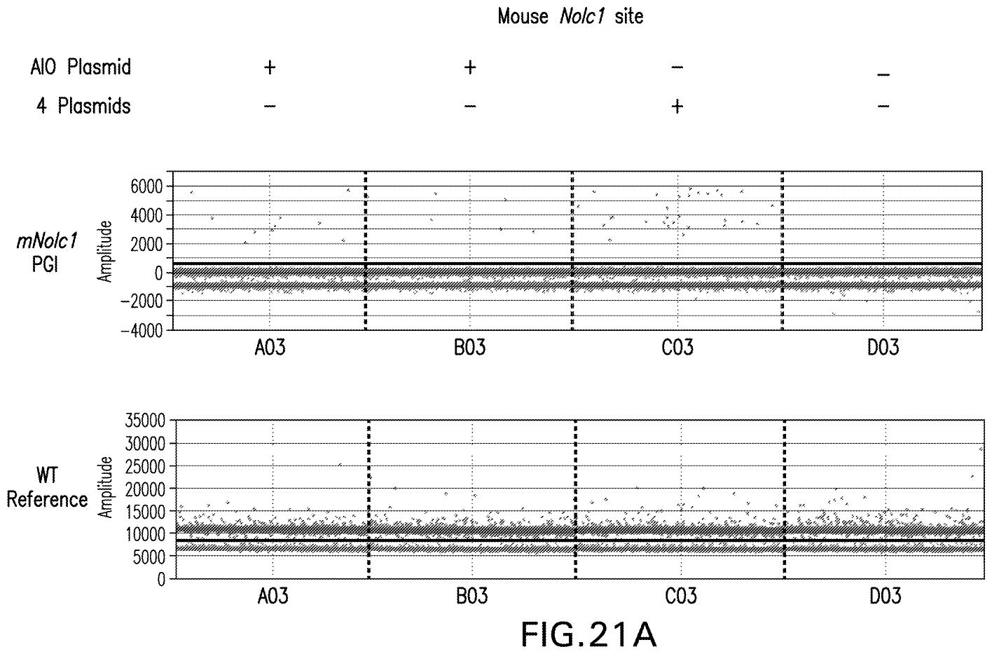

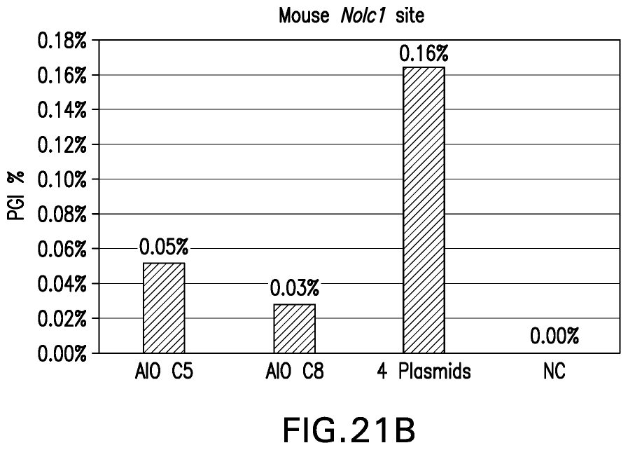

Once expressed B×B1 mediated PGI at the Nolc1 site. In particular, PGI was detected at the Nolc1 site in mouse Hepa 1-6 cells using ddPCR for both single nucleic acid constructs (both clones) but exhibited lower PGI than when PGI components were delivered using a four plasmid system (FIG. 21A and FIG. 21B).

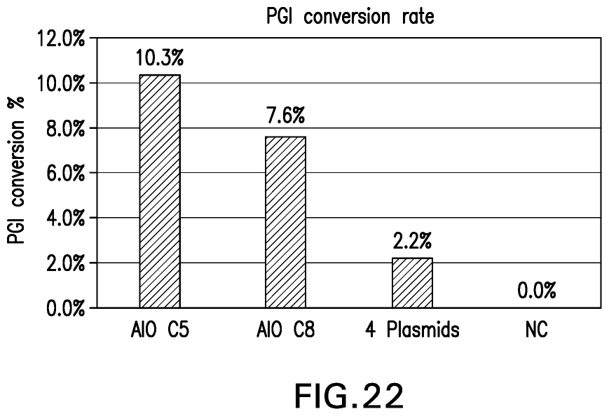

Analysis of PGI conversion rate, calculated as PGI %/(PGI%+BP %), for the data in FIGS. 20A-20B and FIGS. 21A-21B show a higher PGI conversion rate when using the single nucleic acid construct as compared to the four plasmid system (FIG. 22). PGI conversion rate identifies the percentage of beacons where PGI occurred (i.e., integration of the nucleic acid cargo), thereby serving as a proxy for PGI efficiency.

Beacon placement and PGI were confirmed using next generation sequencing (NGS). As shown in FIGS. 23A-23B beacon placement (FIG. 23A) and PGI (FIG. 23B) were higher with the four plasmid system. However, the PGI conversion rate for the data in FIG. 23A and FIG. 23B showed a higher PGI conversion rate for both of the single nucleic acid constructs (both clones) as compared to the four plasmid system (FIG. 24).

Overall, this data shows successful PGI using a single nucleic acid construct in mouse cells. Additionally, this data shows that delivering all of the PGI components in a single nucleic acid construct results in more efficient PGI (i.e., higher PGI conversion rate) than when the delivering the components in separate plasmids.

### 7.7. Example 7: Programmable Gene Insertion with a Single Nucleic Acid Construct (HDAd) in Human Cells

In this example, a single nucleic acid construct having PGI components “all-in-one” (i.e., nucleotide sequence encoding the prime editor fusion protein, nucleotide sequence encoding a first atgRNA, a nucleotide sequence encoding a second atgRNA, a nucleotide sequence encoding an integrase, and a nucleic acid cargo) was compared with a four plasmid system to see which resulted in greater beacon placement and PGI.

The same construct shown in FIG. 18 and used in Example 6 was also used for these experiments. Similarly, the same four plasmid system used in Example 6 was also used for these experiments.

human hHepG2 cells were transfected in a 48 well format with 50,000 cells per well seeded 1 day prior to transfection. Total of 300 ng plasmid DNA was transfected in each well using Lipofectamine 3000 (ThermoFisher) using 3:1 (Lipo3000: DNA) with further experimental details provided in Table 12.

TABLE 12

| | | | | | | | | | Opti- |
|---|---|---|---|---|---|---|---|---|---|
| | | | | | | Total | Lipo3000 | | MEM |
| # | Cells | Plasmid1 | Plasmid2 | Plasmid3 | Plasmid4 | (ng) | (uL) | P3000 | uL |
| 13 | hHepG2 | AdVG012-1 | | | | 300 | 0.9 | 0.6 | 10 + 10 |
| 14 | hHepG2 | duplicate | | | | | | | |
| 15 | hHepG2 | AdVG012-2 | | | | 300 | 0.9 | 0.6 | 10 + 10 |
| 16 | hHepG2 | duplicate | | | | | | | |
| 17 | hHepG2 | PL216 (50 | hF9 | hF9 | CNGNC | 300 | 0.9 | 0.6 | 10 + 10 |
| | | ng) | atgF | atgR | (80 ng) | | | | |
| | | | (100 ng) | (70 ng) | | | | | |
| 18 | hHepG2 | duplicate | | | | | | | |
| 19 | hHepG2 | NC | | | | | | | |
| 20 | hHepG2 | duplicate | | | | | | | |

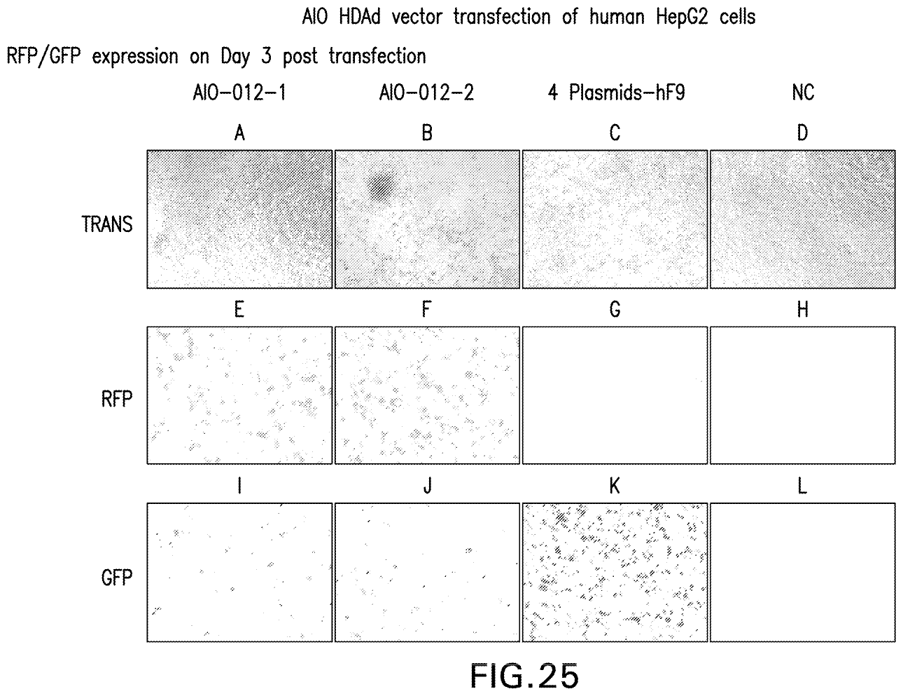

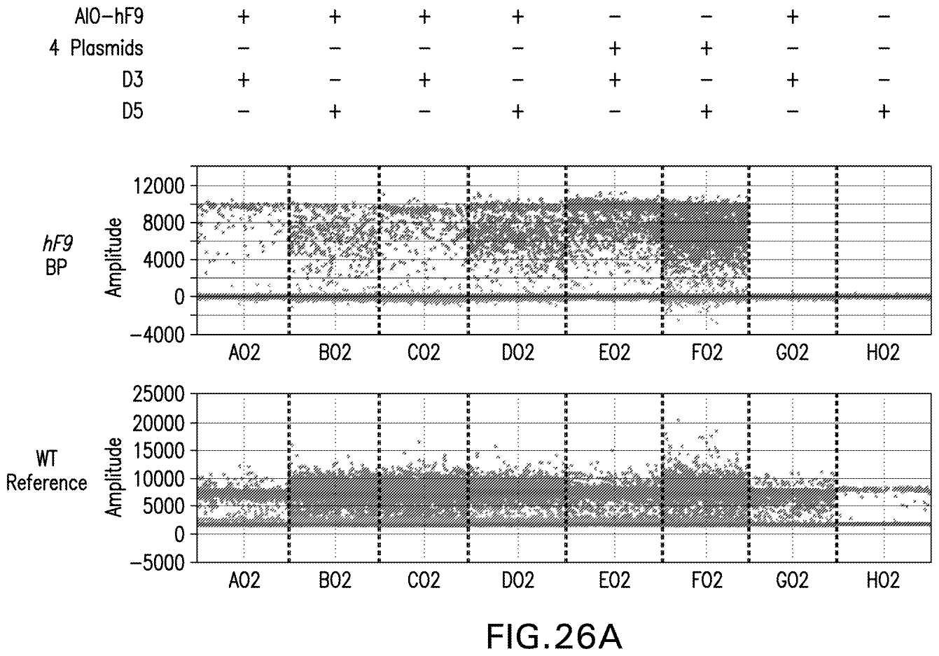

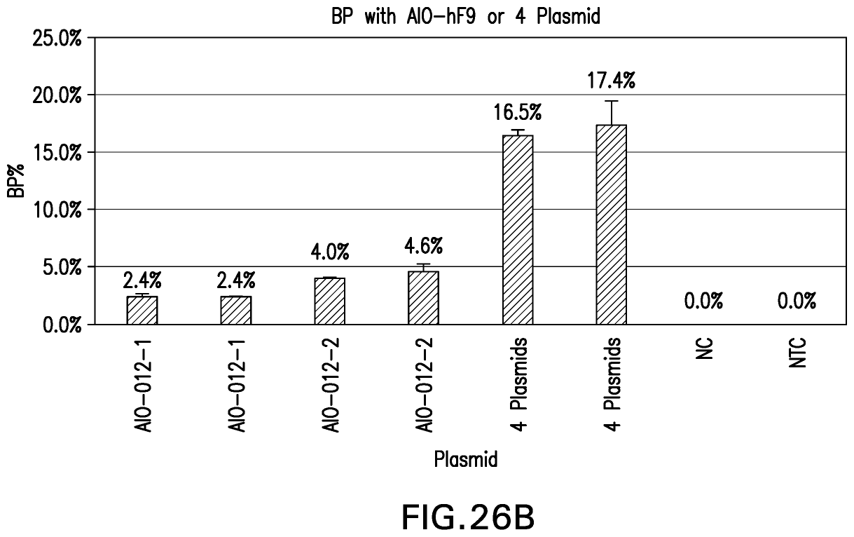

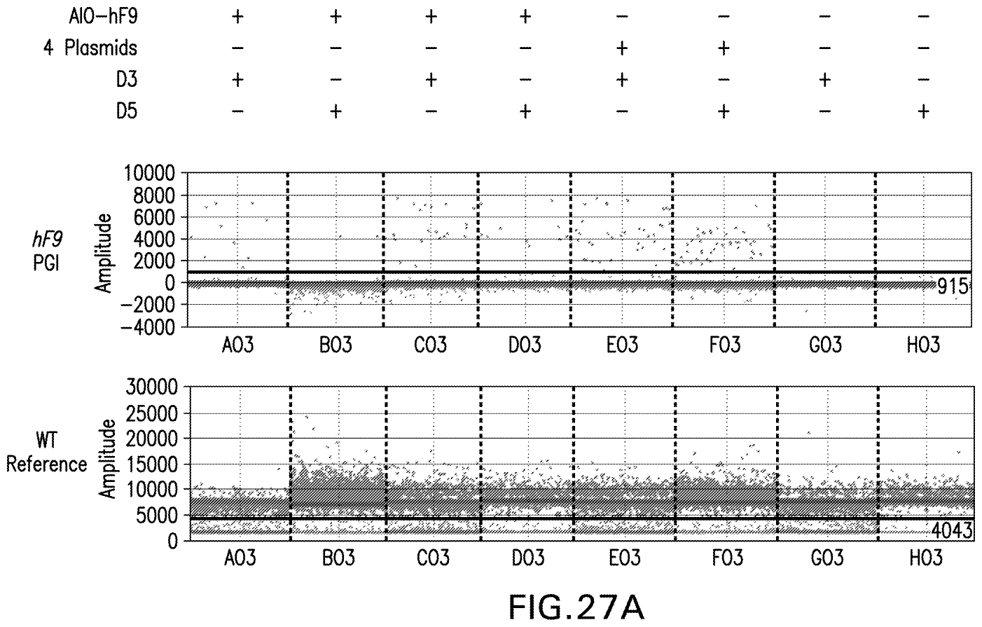

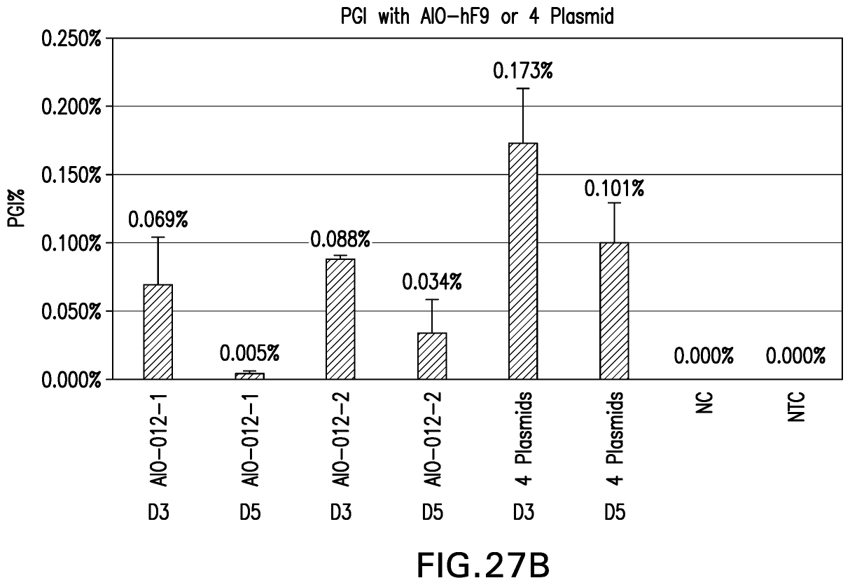

FIGS. 25A-25L show the results at day 2 post transfection. FIGS. 25E and 25F show successful adenovirus transduction for both all-in-one clones (RFP is a marker for all-in-one systems (“AIO-012-1” and “AIO-012-2”) at day 2 post transfection. FIGS. 25K and 25L show GFP expression (marker for four plasmid system (“4plasmids-hF9)) at day 2 post transfection. 72 hours after transfection RNA was collected and subjected to ddPCR and NGS to assess beacon placement and PGI. ddPCR data for beacon placement is shown in FIGS. 26A-26B. ddPCR data for PGI is shown in FIGS. 27A-27B.

Beacon placement at the human Factor IX site in human HepG2 cells was detected using ddPCR (FIG. 26A and FIG. 26B). In particular, transfection of both single nucleic acid constructs (both clones) resulted in beacon placement at the human Factor IX site but was lower than when PGI components were delivered using the four plasmid system.

Once expressed B×B1 mediated PGI at the human Factor IX I site. In particular, PGI was detected at the human Factor IX site using ddPCR for both single nucleic acid constructs (both clones) but exhibited lower PGI than when PGI components were delivered using a four plasmid system (FIG. 27A and FIG. 27B).

Overall, this data shows successful PGI using a single nucleic acid construct in human cells.

### 8. Equivalents and Incorporation by Reference

All references cited herein are incorporated by reference to the same extent as if each individual publication, database entry (e.g. Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57 (b) (1), to relate to each and every individual publication, database entry (e.g. Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

It is an object of the invention not to encompass within the invention any previously known product, process of making the product, or method of using the product such that Applicant reserves the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. 112 (a)) or the EPO (Article 83 of the EPC), such that Applicant reserves the right and hereby disclose a disclaimer of any previously described product, process of making the product, or method of using the product. It may be advantageous in the practice of the invention to be in compliance with Art. 53 (c) EPC and Rule 28 (b) and (c) EPC. Nothing herein is to be construed as a promise. It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

While the invention has been particularly shown and described with reference to a preferred embodiment and various alternate embodiments, it is understood by persons skilled in the relevant art that various changes in form and details can be made therein without departing from the spirit and scope of the invention.

What is claimed is:

1. A nucleic acid construct comprising:
a) a nucleotide sequence encoding a prime editor system;
b) a nucleotide sequence encoding at least a first attachment site-containing guide RNA (atgRNA);
c) a nucleotide sequence encoding at least a first integrase;
d) a nucleic acid cargo;
e) optionally, a nucleotide sequence encoding a nickase guide RNA (ngRNA); and
f) optionally a nucleotide sequence encoding a recombinase.

2. The nucleic acid construct of claim 1, wherein the prime editor system comprises a nucleotide sequence encoding a nickase and a nucleotide sequence encoding a reverse transcriptase.

3. The nucleic acid construct of claim 2, wherein the nucleotide sequence encoding the nickase and the nucleotide sequence encoding the reverse transcriptase are positioned in the construct such that when expressed the gene editor system comprises a fusion protein comprising the nickase and the reverse transcriptase.

4. The nucleic acid construct of any one of claims 1-3, wherein the first integrase that is encoded by a nucleotide sequence in the nucleic acid construct is fused to the prime editor system, the nickase, or the reverse transcriptase by a linker.

5. The nucleic acid construct of any one of claims 1-4, wherein the first atgRNA comprises
(i) a domain that is capable of guiding the prime editor system to a target sequence; and
(ii) a reverse transcriptase (RT) template that comprises at least a portion of a first integration recognition site.

6. The nucleic acid construct of claim 5, wherein the RT template comprises the entirety of the first integration recognition site.

7. The nucleic acid construct of any one of claims 1-6, wherein, upon introducing the nucleic acid construct into a cell, the first atgRNA incorporates the first integrase recognition site into the cell's genome at the target sequence.

8. The nucleic acid construct of any one of claims 1-7, further comprising a second atgRNA.

9. The nucleic acid construct of claim 8, wherein the first atgRNA and the second atgRNA are an at least first pair of atgRNAs, wherein
the at least first pair of atgRNAs have domains that are capable of guiding the prime editor system to a target sequence,
the first atgRNA further includes a first RT template that comprises at least a portion of the first integration recognition site; and
the second atgRNA further includes a second RT template that comprises at least a portion of the first integration recognition site, and
the first atgRNA and the second atgRNAs collectively encode the entirety of the first integration recognition site.

10. The nucleic acid construct of claim 9, wherein, upon introducing the nucleic acid construct into a cell, the first pair of atgRNAs incorporate the first integrase recognition site into the cell's genome at the target sequence.

11. The nucleic acid construct of any one of claims 1-10, further comprising a second integrase recognition site.

12. The nucleic acid construct of claim 11, wherein the second integrase recognition site and the first integrase recognition site are a first cognate pair.

13. The nucleic acid construct of claim 11 or 12, further comprising a third integrase recognition site.

14. The nucleic acid construct of any one of claims 11-13, further comprising a fourth integrase recognition site.

15. The nucleic acid construct of claim 14, wherein the third integrase recognition site and the fourth integrase recognition site are a second cognate pair.

16. The nucleic acid construct of any one of claims 10-15, wherein the second cognate pair has a faster integration rate than the first cognate pair, whereby in the presence of the first integrase the second cognate pair recombines prior to recombination of the first cognate pair.

17. The nucleic acid construct of any one of claims 1-16, further comprising a nucleotide sequence encoding a second integrase.

18. The nucleic acid construct of any one of claims 1-17, wherein the first integrase, the second integrase, or both, are selected from B×B1, Bcec, Sscd, Sacd, Int10, or Pa01.

19. The nucleic acid construct of claim 17 or 18, wherein the first integrase and the second integrase recognize different integration recognition sites.

20. The nucleic acid construct of any one of claims 1-19, further comprising at least a first recombinase recognition site.

21. The nucleic acid construct of claim 20, further comprising a second recombinase recognition site.

22. The nucleic acid construct of any one of claims 1-21, wherein the recombinase is FLP or Cre.

23. The nucleic acid construct of any one of claims 1-22, wherein the nucleic acid cargo comprises at least one of the following: a gene, an expression cassette, a logic gate system, or any combination thereof.

24. The nucleic acid construct of any one of claims 1-23, further comprising a sub-sequence of the nucleic acid construct that is capable of self-circularizing to form a self-circular nucleic acid.

25. The nucleic acid construct of claim 24, wherein the sub-sequence of the nucleic acid construct that is capable of self-circularizing includes the nucleic acid cargo, whereby upon self-circularizing the self-circular nucleic acid comprises the nucleic acid cargo.

26. The nucleic acid construct of claim 24 or 25, wherein the sub-sequence is flanked by the third integrase recognition site and the fourth integrase recognition site.

27. The nucleic acid construct of claim 26, wherein the sub-sequence includes the second integrase recognition site.

28. The nucleic acid construct of any one of claims 25-27, wherein self-circularizing is mediated by recombination of the third integrase recognition site and the fourth integration recognition site by the first integrase.

29. The nucleic acid construct of claim 28, wherein the sub-sequence is flanked by the first recombinase recognition site and the second recombinase recognition site.

30. The nucleic acid construct of claim 29, wherein self-circularizing is mediated by recombination of the first recombinase recognition site and a second recombinase recognition site by the recombinase.

31. The nucleic acid construct of any one of claims 24-30, wherein the self-circular nucleic acid comprises one or more additional integration recognition sites that enable integration of additional nucleic acid cargo.

32. The nucleic acid construct of any of claims 24-31, wherein, upon introducing the nucleic acid construct into a cell and after self-circularizing to form the self-circular nucleic acid, the self-circular nucleic acid comprising the second integrase recognition site is capable of being integrated into the cell's genome at the target sequence that contains the first integrase recognition site.

33. The nucleic acid construct of claim 32, wherein self-circularization to form the self-circular nucleic acid is effected by the first integrase and integration of the self-circular nucleic acid is effected by the second integrase.

34. The nucleic acid construct of any one of claims 1-33, further comprising a 5′ inverted terminal repeat (ITR).

35. The nucleic acid construct of any one of claims 1-34, further comprising a 3′ inverted terminal repeat (ITR).

36. A vector comprising any of the nucleic acid constructs of claims 1-35.

37. The vector of claim 36, wherein the vector is recombinant adenovirus, helper dependent adenovirus, AAV, lentivirus, HSV, annelovirus, retrovirus, Doggybone DNA (dbDNA), minicircle, plasmid, miniDNA, or nanoplasmid.

38. A pharmaceutical composition comprising any of the nucleic acid constructs or vectors of claims 1-37.

39. A method comprising administering an effective amount of a pharmaceutical composition of claim 38 to a patient in need thereof.


SEQUENCE LISTING (559 sequences)

SEQ ID NO: 1 - DNA, 19 bp, synthetic construct
  source 1..19: mol_type = other DNA
ttgagcgggcccccaccgt

SEQ ID NO: 2 - DNA, 21 bp, synthetic construct
  source 1..21: mol_type = other DNA
cagcgggctcagctgatagca

SEQ ID NO: 3 - DNA, 21 bp, synthetic construct
  source 1..21: mol_type = other DNA
cggatggctaaccaagcggcc

SEQ ID NO: 4 - DNA, 393 bp, synthetic construct
  source 1..393: mol_type = other DNA
atgactcactatcaggccttgcttttggacacggaccgggtccagttcggaccggtggtagccctgaacccggctacgctgctcccactgcctgaggaagggctgcaacacaactgccttgatgggacaggtggcggtggtgtcaccgtcaagttcaagtacaagggtgaggaacttgaagttgatattagcaaaatcaagaaggtttggcgcgttggtaaaatgatatcttttacttatgacgacaacggcaagacaggtagaggggcagtgtctgagaaagacgcccccaaggagctgttgcaaatgttggaaaagtctgggaaaaagtctggcggctcaaaaagaaccgccgacggcagcgaattcgagcccaagaagaagaggaaagtc

SEQ ID NO: 5 - DNA, 66 bp, synthetic construct
  source 1..66: mol_type = other DNA
ggaagcggagctactaacttcagcctgctgaagcaggctggcgacgtggaggagaaccctggacct

SEQ ID NO: 6 - DNA, 45 bp, synthetic construct
  source 1..45: mol_type = other DNA
gggggaggaggttctggaggcggaggctccggaggcggagggtca

SEQ ID NO: 7 - DNA, 15 bp, synthetic construct
  source 1..15: mol_type = other DNA
ggaggtggcgggagc

SEQ ID NO: 8 - DNA, 15 bp, synthetic construct
  source 1..15: mol_type = other DNA
cccgcaccagcgcct

SEQ ID NO: 9 - DNA, 45 bp, synthetic construct
  source 1..45: mol_type = other DNA
gaggcagctgccaaggaagccgctgccaaggaggcggccgcaaag

SEQ ID NO: 10 - DNA, 48 bp, synthetic construct
  source 1..48: mol_type = other DNA
agtgggagcgagacccctgggactagcgagtcagctacacccgaaagc

SEQ ID NO: 11 - DNA, 54 bp, synthetic construct
  source 1..54: mol_type = other DNA
ggggggtcaggtggatccggcggaagtggcggatccggtggatctggcggcagt

SEQ ID NO: 12 - DNA, 15 bp, synthetic construct
  source 1..15: mol_type = other DNA
gaagctgctgctaag

SEQ ID NO: 13 - protein, 22 aa, synthetic construct
GSGATNFSLLKQAGDVEENPGP

SEQ ID NO: 14 - protein, 15 aa, synthetic construct
GGGGSGGGGSGGGGS

SEQ ID NO: 15 - protein, 5 aa, synthetic construct
GGGGS

SEQ ID NO: 16 - protein, 5 aa, synthetic construct
PAPAP

SEQ ID NO: 17 - protein, 15 aa, synthetic construct
EAAAKEAAAKEAAAK

SEQ ID NO: 18 - protein, 16 aa, synthetic construct
SGSETPGTSESATPES

SEQ ID NO: 19 - protein, 18 aa, synthetic construct
GGSGGSGGSGGSGGSGGS

SEQ ID NO: 20 - protein, 5 aa, synthetic construct
EAAAK

SEQ ID NO: 21 - protein, 1363 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLS

SEQ ID NO: 22 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 23 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 24 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 25 - protein, 1037 aa, Parvibaculum lavamentivorans
MERIFGFDIGTTSIGFSVIDYSSTQSAGNIQRLGVRIFPEARDPDGTPLNQQRRQKRMMRRQLRRRRIRRKALNETLHEAGFLPAYGSADWPVVMADEPYELRRRGLEEGLSAYEFGRAIYHLAQHRHFKGRELEESDTPDPDVDDEKEAANERAATLKALKNEQTTLGAWLARRPPSDRKRGIHAHRNVVAEEFERLWEVQSKFHPALKSEEMRARISDTIFAQRPVFWRKNTLGECRFMPGEPLCPKGSWLSQQRRMLEKLNNLAIAGGNARPLDAEERDAILSKLQQQASMSWPGVRSALKALYKQRGEPGAEKSLKFNLELGGESKLLGNALEAKLADMFGPDWPAHPRKQEIRHAVHERLWAADYGETPDKKRVIILSEKDRKAHREAAANSFVADFGITGEQAAQLQALKLPTGWEPYSIPALNLFLAELEKGERFGALVNGPDWEGWRRTNFPHRNQPTGEILDKLPSPASKEERERISQLRNPTVVRTQNELRKVVNNLIGLYGKPDRIRIEVGRDVGKSKREREEIQSGIRRNEKQRKKATEDLIKNGIANPSRDDVEKWILWKEGQERCPYTGDQIGFNALFREGRYEVEHIWPRSRSFDNSPRNKTLCRKDVNIEKGNRMPFEAFGHDEDRWSAIQIRLQGMVSAKGGTGMSPGKVKRFLAKTMPEDFAARQLNDTRYAAKQILAQLKRLWPDMGPEAPVKVEAVTGQVTAQLRKLWTLNNILADDGEKTRADHRHHAIDALTVACTHPGMTNKLSRYWQLRDDPRAEKPALTPPWDTIRADAEKAVSEIVVSHRVRKKVSGPLHKETTYGDTGTDIKTKSGTYRQFVTRKKIESLSKGELDEIRDPRIKEIVAAHVAGRGGDPKKAFPPYPCVSPGGPEIRKVRLTSKQQLNLMAQTGNGYADLGSNHHIAIYRLPDGKADFEIVSLFDASRRLAQRNPIVQRTRADGASFVMSLAAGEAIMIPEGSKKGIWIVQGVWASGQVVLERDTDADHSTTTRPMPNPILKDDAKKVSIDPIGRVRPSND

SEQ ID NO: 26 - protein, 1084 aa, Corynebacterium diphtheriae
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 27 - protein, 1130 aa, unidentified
MTNGKILGLDIGIASVGVGIIEAKTGKVVHANSRLFSAANAENNAERRGFRGSRRLNRRKKHRVKRVRDLFEKYGIVTDFRNLNLNPYELRVKGLTEQLKNEELFAALRTISKRRGISYLDDAEDDSTGSTDYAKSIDENRRLLKNKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCNFYPDEYRASKASYTAQEYNFLNDLNNLKVSTETGKLSTEQKESLVEFAKNTATLGPAKLLKEIAKILDCKVDEIKGYREDDKGKPDLHTFEPYRKLKFNLESINIDDLSREVIDKLADILTLNTEREGIEDAIKRNLPNQFTEEQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNSSDKLPDEVFHGNKQLETKIRLWYQQGERCLYSGKPISIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGKNQVVDKQTGEILSVSDDEYKELVFQPPYQGFVNTISSKGFEDEILFSYQVDSKYNRKVSDATIYSTRKAKIGKDKKEETYVLGKIKDIYSQNGFDTFIKKYNKDKTQFLMYQKDSLTWENVIEVILRDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKKGDKPKLDFKNNKK

SEQ ID NO: 28 - protein, 1082 aa, Neisseria cinerea
MAAFKPNPMNYILGLDIGIASVGWAIVEIDEEENPIRLIDLGVRVFERAEVPKTGDSLAAARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNTHALQTGDFRTPAELALNKFEKESGHIRNQRGDYSHTFNRKDLQAELNLLFEKQKEFGNPHVSDGLKEGIETLLMTQRPALSGDAVQKMLGHCTFEPTEPKAAKNTYTAERFVWLTKLNNLRILEQGSERPLTDTERATLMDEPYRKSKLTYAQARKLLDLDDTAFFKGLRYGKDNAEASTLMEMKAYHAISRALEKEGLKDKKSPLNLSPELQDEIGTAFSLFKTDEDITGRLKDRVQPEILEALLKHISFDKFVQISLKALRRIVPLMEQGNRYDEACTEIYGDHYGKKNTEEKIYLPPIPADEIRNPVVLRALSQARKVINGVVRRYGSPARIHIETAREVGKSFKDRKEIEKRQEENRKDREKSAAKFREYFPNFVGEPKSKDILKLRLYEQQHGKCLYSGKEINLGRLNEKGYVEIDHALPFSRTWDDSFNNKVLALGSENQNKGNQTPYEYFNGKDNSREWQEFKARVETSRFPRSKKQRILLQKFDEDGFKERNLNDTRYINRFLCQFVADHMLLTGKGKRRVFASNGQITNLLRGFWGLRKVRAENDRHHALDAVVVACSTIAMQQKITRFVRYKEMNAFDGKTIDKETGEVLHQKAHFPQPWEFFAQEVMIRVFGKPDGKPEFEEADTPEKLRTLLAEKLSSRPEAVHKYVTPLFISRAPNRKMSGQGHMETVKSAKRLDEGISVLRVPLTQLKLKDLEKMVNREREPKLYEALKARLEAHKDDPAKAFAEPFYKYDKAGNRTQQVKAVRVEQVQKTGVWVHNHNGIADNATIVRVDVFEKGGKYYLVPIYSWQVAKGILPDRAVVQGKDEEDWTVMDDSFEFKFVLYANDLIKLTAKKNEFLGYFVSLNRATGAIDIRTHDTDSTKGKNGIFQSVGVKTALSFQKYQIDELGKEIRPCRLKKRPPVR

SEQ ID NO: 29 - protein, 1115 aa, unidentified
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - protein, 1003 aa, Campylobacter lari
MRILGFDIGINSIGWAFVENDELKDCGVRIFTKAENPKNKESLALPRRNARSSRRRLKRRKARLIAIKRILAKELKLNYKDYVAADGELPKAYEGSLASVYELRYKALTQNLETKDLARVILHIAKHRGYMNKNEKKSNDAKKGKILSALKNNALKLENYQSVGEYFYKEFFQKYKKNTKNFIKIRNTKDNYNNCVLSSDLEKELKLILEKQKEFGYNYSEDFINEILKVAFFQRPLKDFSHLVGACTFFEEEKRACKNSYSAWEFVALTKIINEIKSLEKISGEIVPTQTINEVLNLILDKGSITYKKFRSCINLHESISFKSLKYDKENAENAKLIDFRKLVEFKKALGVHSLSRQELDQISTHITLIKDNVKLKTVLEKYNLSNEQINNLLEIEFNDYINLSFKALGMILPLMREGKRYDEACEIANLKPKTVDEKKDFLPAFCDSIFAHELSNPVVNRAISEYRKVLNALLKKYGKVHKIHLELARDVGLSKKAREKIEKEQKENQAVNAWALKECENIGLKASAKNILKLKLWKEQKEICIYSGNKISIEHLKDEKALEVDHIYPYSRSFDDSFINKVLVFTKENQEKLNKTPFEAFGKNIEKWSKIQTLAQNLPYKKKNKILDENFKDKQQEDFISRNLNDTRYIATLIAKYTKEYLNFLLLSENENANLKSGEKGSKIHVQTISGMLTSVLRHTWGFDKKDRNNHLHHALDAIIVAYSTNSIIKAFSDFRKNQELLKARFYAKELTSDNYKHQVKFFEPFKSFREKILSKIDEIFVSKPPRKRARRALHKDTFHSENKIIDKCSYNSKEGLQIALSCGRVRKIGTKYVENDTIVRVDIFKKQNKFYAIPIYAMDFALGILPNKIVITGKDKNNNPKQWQTIDESYEFCFSLYKNDLILLQKKNMQEPEFAYYNDFSISTSSICVEKHDNKFENLTSNQKLLFSNAKEGSVKVESLGIQNLKVFEKYIITPLGDKIKADFQPRENISLKTSKKYGLR

SEQ ID NO: 31 - protein, 1374 aa, unidentified
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 32 - protein, 1368 aa, unidentified
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIAMARENQTTQKGQKNSRERMKRIEEGIKELGSQILKEHPVENTQLQNEKLYLYYLQNGRDMYVDQELDINRLSDYDVDAIVPQSFLKDDSIDAKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHAAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 33 - protein, 1422 aa, Sutterella wadsworthensis
MTQSERRFSCSIGIDMGAKYTGVFYALFDREELPTNLNSKAMTLVMPETGPRYVQAQRTAVRHRLRGQKRYTLARKLAFLVVDDMIKKQEKRLTDEEWKRGREALSGLLKRRGYSRPNADGEDLTPLENVRADVFAAHPAFSTYFSEVRSLAEQWEEFTANISNVEKFLGDPNIPADKEFIEFAVAEGLIDKTEKKAYQSALSTLRANANVLTGLRQMGHKPRSEYFKAIEADLKKDSRLAKINEAFGGAERLARLLGNLSNLQLRAERWYFNAPDIMKDRGWEPDRFKKTLVRAFKFFHPAKDQNKQHLELIKQIENSEDIIETLCTLDPNRTIPPYEDQNNRRPPLDQTLLLSPEKLTRQYGEIWKTWSARLTSAEPTLAPAAEILERSTDRKSRVAVNGHEPLPTLAYQLSYALQRAFDRSKALDPYALRALAAGSKSNKLTSARTALENCIGGQNVKTFLDCARRYYREADDAKVGLWFDNADGLLERSDLHPPMKKKILPLLVANILQTDETTGQKFLDEIWRKQIKGRETVASRCARIETVRKSFGGGFNIAYNTAQYREVNKLPRNAQDKELLTIRDRVAETADFIAANLGLSDEQKRKFANPFSLAQFYTLIETEVSGFSATTLAVHLENAWRMTIKDAVINGETVRAAQCSRLPAETARPFDGLVRRLVDRQAWEIAKRVSTDIQSKVDFSNGIVDVSIFVEENKFEFSASVADLKKNKRVKDKMLSEAEKLETRWLIKNERIKKASRGTCPYTGDRLAEGGEIDHILPRSLIKDARGIVFNAEPNLIYASSRGNQLKKNQRYSLSDLKANYRNEIFKTSNIAAITAEIEDVVTKLQQTHRLKFFDLLNEHEQDCVRHALFLDDGSEARDAVLELLATQRRTRVNGTQIWMIKNLANKIREELQNWCKTTNNRLHFQAAATNVSDAKNLRLKLAQNQPDFEKPDIQPIASHSIDALCSFAVGSADAERDQNGFDYLDGKTVLGLYPQSCEVIHLQAKPQEEKSHFDSVAIFKEGIYAEQFLPIFTLNEKIWIGYETLNAKGERCGAIEVSGKQPKELLEMLAPFFNKPVGDLSAHATYRILKKPAYEFLAKAALQPLSAEEKRLAALLDALRYCTSRKSLMSLFMAANGKSLKKREDVLKPKLFQLKVELKGEKSFKLNGSLTLPVKQDWLRICDSPELADAFGKPCSADELTSKLARIWKRPVMRDLAHAPVRREFSLPAIDNPSGGFRIRRTNLFGNELYQVHAINAKKYRGFASAGSNVDWSKGILFNELQHENLTECGGRFITSADVTPMSEWRKVVAEDNLSIWIAPGTEGRRYVRVETTFIQASHWFEQSVENWAITSPLSLPASFKVDKPAEFQKAVGTELSELLGQPRSEIFIENVGNAKHIRFWYIVVSSNKKMNESYNNVSKS

SEQ ID NO: 34 - protein, 1372 aa, Legionella pneumophila
MESSQILSPIGIDLGGKFTGVCLSHLEAFAELPNHANTKYSVILIDHNNFQLSQAQRRATRHRVRNKKRNQFVKRVALQLFQHILSRDLNAKEETALCHYLNNRGYTYVDTDLDEYIKDETTINLLKELLPSESEHNFIDWFLQKMQSSEFRKILVSKVEEKKDDKELKNAVKNIKNFITGFEKNSVEGHRHRKVYFENIKSDITKDNQLDSIKKKIPSVCLSNLLGHLSNLQWKNLHRYLAKNPKQFDEQTFGNEFLRMLKNFRHLKGSQESLAVRNLIQQLEQSQDYISILEKTPPEITIPPYEARTNTGMEKDQSLLLNPEKLNNLYPNWRNLIPGIIDAHPFLEKDLEHTKLRDRKRIISPSKQDEKRDSYILQRYLDLNKKIDKFKIKKQLSFLGQGKQLPANLIETQKEMETHFNSSLVSVLIQIASAYNKEREDAAQGIWFDNAFSLCELSNINPPRKQKILPLLVGAILSEDFINNKDKWAKFKIFWNTHKIGRTSLKSKCKEIEEARKNSGNAFKIDYEEALNHPEHSNNKALIKIIQTIPDIIQAIQSHLGHNDSQALIYHNPFSLSQLYTILETKRDGFHKNCVAVTCENYWRSQKTEIDPEISYASRLPADSVRPFDGVLARMMQRLAYEIAMAKWEQIKHIPDNSSLLIPIYLEQNRFEFEESFKKIKGSSSDKTLEQAIEKQNIQWEEKFQRIINASMNICPYKGASIGGQGEIDHIYPRSLSKKHFGVIFNSEVNLIYCSSQGNREKKEEHYLLEHLSPLYLKHQFGTDNVSDIKNFISQNVANIKKYISFHLLTPEQQKAARHALFLDYDDEAFKTITKFLMSQQKARVNGTQKFLGKQIMEFLSTLADSKQLQLEFSIKQITAEEVHDHRELLSKQEPKLVKSRQQSFPSHAIDATLTMSIGLKEFPQFSQELDNSWFINHLMPDEVHLNPVRSKEKYNKPNISSTPLFKDSLYAERFIPVWVKGETFAIGFSEKDLFEIKPSNKEKLFTLLKTYSTKNPGESLQELQAKSKAKWLYFPINKTLALEFLHHYFHKEIVTPDDTTVCHFINSLRYYTKKESITVKILKEPMPVLSVKFESSKKNVLGSFKHTIALPATKDWERLFNHPNFLALKANPAPNPKEFNEFIRKYFLSDNNPNSDIPNNGHNIKPQKHKAVRKVFSLPVIPGNAGTMMRIRRKDNKGQPLYQLQTIDDTPSMGIQINEDRLVKQEVLMDAYKTRNLSTIDGINNSEGQAYATFDNWLTLPVSTFKPEIIKLEMKPHSKTRRYIRITQSLADFIKTIDEALMIKPSDSIDDPLNMPNEIVCKNKLFGNELKPRDGKMKIVSTGKIVTYEFESDSTPQWIQTLYVTQLKKQP

SEQ ID NO: 35 - protein, 1395 aa, Treponema denticola
MKKEIKDYFLGLDVGTGSVGWAVTDTDYKLLKANRKDLWGMRCFETAETAEVRRLHRGARRRIERRKKRIKLLQELFSQEIAKTDEGFFQRMKESPFYAEDKTILQENTLFNDKDFADKTYHKAYPTINHLIKAWIENKVKPDPRLLYLACHNIIKKRGHFLFEGDFDSENQFDTSIQALFEYLREDMEVDIDADSQKVKEILKDSSLKNSEKQSRLNKILGLKPSDKQKKAITNLISGNKINFADLYDNPDLKDAEKNSISFSKDDFDALSDDLASILGDSFELLLKAKAVYNCSVLSKVIGDEQYLSFAKVKIYEKHKTDLTKLKNVIKKHFPKDYKKVFGYNKNEKNNNNYSGYVGVCKTKSKKLIINNSVNQEDFYKFLKTILSAKSEIKEVNDILTEIETGTFLPKQISKSNAEIPYQLRKMELEKILSNAEKHFSFLKQKDEKGLSHSEKIIMLLTFKIPYYIGPINDNHKKFFPDRCWVVKKEKSPSGKTTPWNFFDHIDKEKTAEAFITSRTNFCTYLVGESVLPKSSLLYSEYTVLNEINNLQIIIDGKNICDIKLKQKIYEDLFKKYKKITQKQISTFIKHEGICNKTDEVIILGIDKECTSSLKSYIELKNIFGKQVDEISTKNMLEEIIRWATIYDEGEGKTILKTKIKAEYGKYCSDEQIKKILNLKFSGWGRLSRKFLETVTSEMPGFSEPVNIITAMRETQNNLMELLSSEFTFTENIKKINSGFEDAEKQFSYDGLVKPLFLSPSVKKMLWQTLKLVKEISHITQAPPKKIFIEMAKGAELEPARTKTRLKILQDLYNNCKNDADAFSSEIKDLSGKIENEDNLRLRSDKLYLYYTQLGKCMYCGKPIEIGHVFDTSNYDIDHIYPQSKIKDDSISNRVLVCSSCNKNKEDKYPLKSEIQSKQRGFWNFLQRNNFISLEKLNRLTRATPISDDETAKFIARQLVETRQATKVAAKVLEKMFPETKIVYSKAETVSMFRNKFDIVKCREINDFHHAHDAYLNIVVGNVYNTKFTNNPWNFIKEKRDNPKIADTYNYYKVFDYDVKRNNITAWEKGKTIITVKDMLKRNTPIYTRQAACKKGELFNQTIMKKGLGQHPLKKEGPFSNISKYGGYNKVSAAYYTLIEYEEKGNKIRSLETIPLYLVKDIQKDQDVLKSYLTDLLGKKEFKILVPKIKINSLLKINGFPCHITGKTNDSFLLRPAVQFCCSNNEVLYFKKIIRFSEIRSQREKIGKTISPYEDLSFRSYIKENLWKKTKNDEIGEKEFYDLLQKKNLEIYDMLLTKHKDTIYKKRPNSATIDILVKGKEKFKSLIIENQFEVILEILKLFSATRNVSDLQHIGGSKYSGVAKIGNKISSLDNCILIYQSITGIFEKRIDLLKV

SEQ ID NO: 36 - protein, 1365 aa, Filifactor alocis
MTKEYYLGLDVGTNSVGWAVTDSQYNLCKFKKKDMWGIRLFESANTAKDRRLQRGNRRRLERKKQRIDLLQEIFSPEICKIDPTFFIRLNESRLHLEDKSNDFKYPLFIEKDYSDIEYYKEFPTIFHLRKHLIESEEKQDIRLIYLALHNIIKTRGHFLIDGDLQSAKQLRPILDTFLLSLQEEQNLSVSLSENQKDEYEEILKNRSIAKSEKVKKLKNLFEISDELEKEEKKAQSAVIENFCKFIVGNKGDVCKFLRVSKEELEIDSFSFSEGKYEDDIVKNLEEKVPEKVYLFEQMKAMYDWNILVDILETEEYISFAKVKQYEKHKTNLRLLRDIILKYCTKDEYNRMFNDEKEAGSYTAYVGKLKKNNKKYWIEKKRNPEEFYKSLGKLLDKIEPLKEDLEVLTMMIEECKNHTLLPIQKNKDNGVIPHQVHEVELKKILENAKKYYSFLTETDKDGYSVVQKIESIFRFRIPYYVGPLSTRHQEKGSNVWMVRKPGREDRIYPWNMEEIIDFEKSNENFITRMTNKCTYLIGEDVLPKHSLLYSKYMVLNELNNVKVRGKKLPTSLKQKVFEDLFENKSKVTGKNLLEYLQIQDKDIQIDDLSGFDKDFKTSLKSYLDFKKQIFGEEIEKESIQNMIEDIIKWITIYGNDKEMLKRVIRANYSNQLTEEQMKKITGFQYSGWGNFSKMFLKGISGSDVSTGETFDIITAMWETDNNLMQILSKKFTFMDNVEDFNSGKVGKIDKITYDSTVKEMFLSPENKRAVWQTIQVAEEIKKVMGCEPKKIFIEMARGGEKVKKRTKSRKAQLLELYAACEEDCRELIKEIEDRDERDFNSMKLFLYYTQFGKCMYSGDDIDINELIRGNSKWDRDHIYPQSKIKDDSIDNLVLVNKTYNAKKSNELLSEDIQKKMHSFWLSLLNKKLITKSKYDRLTRKGDFTDEELSGFIARQLVETRQSTKAIADIFKQIYSSEVVYVKSSLVSDFRKKPLNYLKSRRVNDYHHAKDAYLNIVVGNVYNKKFTSNPIQWMKKNRDTNYSLNKVFEHDVVINGEVIWEKCTYHEDTNTYDGGTLDRIRKIVERDNILYTEYAYCEKGELFNATIQNKNGNSTVSLKKGLDVKKYGGYFSANTSYFSLIEFEDKKGDRARHIIGVPIYIANMLEHSPSAFLEYCEQKGYQNVRILVEKIKKNSLLIINGYPLRIRGENEVDTSFKRAIQLKLDQKNYELVRNIEKFLEKYVEKKGNYPIDENRDHITHEKMNQLYEVLLSKMKKFNKKGMADPSDRIEKSKPKFIKLEDLIDKINVINKMLNLLRCDNDTKADLSLIELPKNAGSFVVKKNTIGKSKIILVNQSVTGLYENRREL

SEQ ID NO: 37 - protein, 1334 aa, Staphylococcus pseudintermedius
MGRKPYILSLDIGTGSVGYACMDKGFNVLKYHDKDALGVYLFDGALTAQERRQFRTSRRRKNRRIKRLGLLQELLAPLVQNPNFYQFQRQFAWKNDNMDFKNKSLSEVLSFLGYESKKYPTIYHLQEALLLKDEKFDPELIYMALYHLVKYRGHFLFDHLKIENLTNNDNMHDFVELIETYENLNNIKLNLDYEKTKVIYEILKDNEMTKNDRAKRVKNMEKKLEQFSIMLLGLKFNEGKLFNHADNAEELKGANQSHTFADNYEENLTPFLTVEQSEFIERANKIYLSLTLQDILKGKKSMAMSKVAAYDKFRNELKQVKDIVYKADSTRTQFKKIFVSSKKSLKQYDATPNDQTFSSLCLFDQYLIRPKKQYSLLIKELKKIIPQDSELYFEAENDTLLKVLNTTDNASIPMQINLYEAETILRNQQKYHAEITDEMIEKVLSLIQFRIPYYVGPLVNDHTASKFGWMERKSNESIKPWNFDEVVDRSKSATQFIRRMTNKCSYLINEDVLPKNSLLYQEMEVLNELNATQIRLQTDPKNRKYRMMPQIKLFAVEHIFKKYKTVSHSKFLEIMLNSNHRENFMNHGEKLSIFGTQDDKKFASKLSSYQDMTKIFGDIEGKRAQIEEIIQWITIFEDKKILVQKLKECYPELTSKQINQLKKLNYSGWGRLSEKLLTHAYQGHSIIELLRHSDENFMEILTNDVYGFQNFIKEENQVQSNKIQHQDIANLTTSPALKKGIWSTIKLVRELTSIFGEPEKIIMEFATEDQQKGKKQKSRKQLWDDNIKKNKLKSVDEYKYIIDVANKLNNEQLQQEKLWLYLSQNGKCMYSGQSIDLDALLSPNATKHYEVDHIFPRSFIKDDSIDNKVLVIKKMNQTKGDQVPLQFIQQPYERIAYWKSLNKAGLISDSKLHKLMKPEFTAMDKEGFIQRQLVETRQISVHVRDFLKEEYPNTKVIPMKAKMVSEFRKKFDIPKIRQMNDAHHAIDAYLNGVVYHGAQLAYPNVDLFDFNFKWEKVREKWKALGEFNTKQKSRELFFFKKLEKMEVSQGERLISKIKLDMNHFKINYSRKLANIPQQFYNQTAVSPKTAELKYESNKSNEVVYKGLTPYQTYVVAIKSVNKKGKEKMEYQMIDHYVFDFYKFQNGNEKELALYLAQRENKDEVLDAQIVYSLNKGDLLYINNHPCYFVSRKEVINAKQFELTVEQQLSLYNVMNNKETNVEKLLIEYDFIAEKVINEYHHYLNSKLKEKRVRTFFSESNQTHEDFIKALDELFKVVTASATRSDKIGSRKNSMTHRAFLGKGKDVKIAYTSISGLKTTKPKSLFKLAESRNEL

SEQ ID NO: 38 - protein, 1375 aa, Lactobacillus johnsonii
MTKIKDDYIVGLDIGTDSCGWVAMNSNNDILKLQGKTAIGSRLFEGGKSAAERRLFRTTHRRIKRRRWRLKLLEEFFDPYMAEVDPYFFARLKESGLSPLDKRKTVSSIVFPTSAEDKKFYDDYPTIYHLRYKLMTEDEKFDLREVYLAIHHIIKYRGNFLYNTSVKDFKASKIDVKSSIEKLNELYENLGLDLNVEFNISNTAEIEKVLKDKQIFKRDKVKKIAELFAIKTDNKEQSKRIKDISKQVANAVLGYKTRFDTIALKEISKDELSDWNFKLSDIDADSKFEALMGNLDENEQAILLTIKELFNEVTLNGIVEDGNTLSESMINKYNDHRDDLKLLKEVIENHIDRKKAKELALAYDLYVNNRHGQLLQAKKKLGKIKPRSKEDFYKVVNKNLDDSRASKEIKKKIELDSFMPKQRTNANGVIPYQLQQLELDKIIENQSKYYPFLKEINPVSSHLKEAPYKLDELIRFRVPYYVGPLISPNESTKDIQTKKNQNFAWMIRKEEGRITPWNFDQKVDRIESANKFIKRMTTKDTYLFGEDVLPANSLLYQKFTVLNELNNIRINGKRISVDLKQEIYENLFKKHTTVTVKKLENYLKENHNLVKVEIKGLADEKKFNSGLTTYNRFKNLNIFDNQIDDLKYRNDFEKIIEWSTIFEDKSIYKEKLRSIDWLNEKQINALSNIRLQGWGRLSKKLLAQLHDHNGQTIIEQLWDSQNNFMQIVTQADFKDAIAKANQNLLVATSVEDILNNAYTSPANKKAIRQVIKVVDDIVKAASGKVPKQIAIEFTRDADENPKRSQTRGSKLQKVYKDLSTELASKTIAEELNEAIKDKKLVQDKYYLYFMQLGRDAYTGEPINIDEIQKYDIDHILPQSFIKDDALDNRVLVSRAVNNGKSDNVPVKLFGNEMAANLGMTIRKMWEEWKNIGLISKTKYNNLLTDPDHINKYKSAGFIRRQLVETSQIIKLVSTILQSRYPNTEIITVKAKYNHYLREKFDLYKSREVNDYHHAIDAYLSAICGNLLYQNYPNLRPFFVYGQYKKFSSDPDKEKAIFNKTRKFSFISQLLKNKSENSKEIAKKLKRAYQFKYMLVSRETETRDQEMFKMTVYPRFSHDTVKAPRNLIPKKMGMSPDIYGGYTNNSDAYMVIVRIDKKKGTEYKILGIPTRELVNLKKAEKEDHYKSYLKEILTPRILYNKNGKRDKKITSFEIVKSKIPYKQVIQDGDKKFMLGSSTYVYNAKQLTLSTESMKAITNNFDKDSDENDALIKAYDEILDKVDKYLPLFDINKFREKLHSGREKFIKLSLEDKKDTILKVLEGLHDNAVMTKIPTIGLSTPLGFMQFPNGVILSENAKLIYQSPTGLFKKSVKISDL

SEQ ID NO: 39 - protein, 1269 aa, Mycoplasma gallisepticum
MNNSIKSKPEVTIGLDLGVGSVGWAIVDNETNIIHHLGSRLFSQAKTAEDRRSFRGVRRLIRRRKYKLKRFVNLIWKYNSYFGFKNKEDILNNYQEQQKLHNTVLNLKSEALNAKIDPKALSWILHDYLKNRGHFYEDNRDFNVYPTKELAKYFDKYGYYKGIIDSKEDNDNKLEEELTKYKFSNKHWLEEVKKVLSNQTGLPEKFKEEYESLFSYVRNYSEGPGSINSVSPYGIYHLDEKEGKVVQKYNNIWDKTIGKCNIFPDEYRAPKNSPIAMIFNEINELSTIRSYSIYLTGWFINQEFKKAYLNKLLDLLIKTNGEKPIDARQFKKLREETIAESIGKETLKDVENEEKLEKEDHKWKLKGLKLNTNGKIQYNDLSSLAKFVHKLKQHLKLDFLLEDQYATLDKINFLQSLFVYLGKHLRYSNRVDSANLKEFSDSNKLFERILQKQKDGLFKLFEQTDKDDEKILAQTHSLSTKAMLLAITRMTNLDNDEDNQKNNDKGWNFEAIKNFDQKFIDITKKNNNLSLKQNKRYLDDRFINDAILSPGVKRILREATKVFNAILKQFSEEYDVTKVVIELARELSEEKELENTKNYKKLIKKNGDKISEGLKALGISEDEIKDILKSPTKSYKFLLWLQQDHIDPYSLKEIAFDDIFTKTEKFEIDHIIPYSISFDDSSSNKLLVLAESNQAKSNQTPYEFISSGNAGIKWEDYEAYCRKFKDGDSSLLDSTQRSKKFAKMMKTDTSSKYDIGFLARNLNDTRYATIVFRDALEDYANNHLVEDKPMFKVVCINGSVTSFLRKNFDDSSYAKKDRDKNIHHAVDASIISIFSNETKTLFNQLTQFADYKLFKNTDGSWKKIDPKTGVVTEVTDENWKQIRVRNQVSEIAKVIEKYIQDSNIERKARYSRKIENKTNISLFNDTVYSAKKVGYEDQIKRKNLKTLDIHESAKENKNSKVKRQFVYRKLVNVSLLNNDKLADLFAEKEDILMYRANPWVINLAEQIFNEYTENKKIKSQNVFEKYMLDLTKEFPEKFSEFLVKSMLRNKTAIIYDDKKNIVHRIKRLKMLSSELKENKLSNVIIRSKNQSGTKLSYQDTINSLALMIMRSIDPTAKKQYIRVPLNTLNLHLGDHDFDLHNMDAYLKKPKFVKYLKANEIGDEYKPWRVLTSGTLLIHKKDKKLMYISSFQNLNDVIEIKNLIETEYKENDDSDSKKKKKANRFLMTLSTILNDYILLDAKDNFDILGLSKNRIDEILNSKLGLDKIVK

SEQ ID NO: 40 - protein, 1003 aa, unidentified; identical to SEQ ID NO: 30

SEQ ID NO: 41 - protein, 1120 aa, unidentified
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLM

SEQ ID NO: 42 - protein, 1415 aa, Bergeyella zoohelcum
MKHILGLDLGTNSIGWALIERNIEEKYGKIIGMGSRIVPMGAELSKFEQGQAQTKNADRRTNRGARRLNKRYKQRRNKLIYILQKLDMLPSQIKLKEDFSDPNKIDKITILPISKKQEQLTAFDLVSLRVKALTEKVGLEDLGKIIYKYNQLRGYAGGSLEPEKEDIFDEEQSKDKKNKSFIAFSKIVFLGEPQEEIFKNKKLNRRAIIVETEEGNFEGSTFLENIKVGDSLELLINISASKSGDTITIKLPNKTNWRKKMENIENQLKEKSKEMGREFYISEFLLELLKENRWAKIRNNTILRARYESEFEAIWNEQVKHYPFLENLDKKTLIEIVSFIFPGEKESQKKYRELGLEKGLKYIIKNQVVFYQRELKDQSHLISDCRYEPNEKAIAKSHPVFQEYKVWEQINKLIVNTKIEAGTNRKGEKKYKYIDRPIPTALKEWIFEELQNKKEITFSAIFKKLKAEFDLREGIDFLNGMSPKDKLKGNETKLQLQKSLGELWDVLGLDSINRQIELWNILYNEKGNEYDLTSDRTSKVLEFINKYGNNIVDDNAEETAIRISKIKFARAYSSLSLKAVERILPLVRAGKYFNNDFSQQLQSKILKLLNENVEDPFAKAAQTYLDNNQSVLSEGGVGNSIATILVYDKHTAKEYSHDELYKSYKEINLLKQGDLRNPLVEQIINEALVLIRDIWKNYGIKPNEIRVELARDLKNSAKERATIHKRNKDNQTINNKIKETLVKNKKELSLANIEKVKLWEAQRHLSPYTGQPIPLSDLFDKEKYDVDHIIPISRYFDDSFTNKVISEKSVNQEKANRTAMEYFEVGSLKYSIFTKEQFIAHVNEYFSGVKRKNLLATSIPEDPVQRQIKDTQYIAIRVKEELNKIVGNENVKTTTGSITDYLRNHWGLTDKFKLLLKERYEALLESEKFLEAEYDNYKKDFDSRKKEYEEKEVLFEEQELTREEFIKEYKENYIRYKKNKLIIKGWSKRIDHRHHAIDALIVACTEPAHIKRLNDLNKVLQDWLVEHKSEFMPNFEGSNSELLEEILSLPENERTEIFTQIEKFRAIEMPWKGFPEQVEQKLKEIIISHKPKDKLLLQYNKAGDRQIKLRGQLHEGTLYGISQGKEAYRIPLTKFGGSKFATEKNIQKIVSPFLSGFIANHLKEYNNKKEEAFSAEGIMDLNNKLAQYRNEKGELKPHTPISTVKIYYKDPSKNKKKKDEEDLSLQKLDREKAFNEKLYVKTGDNYLFAVLEGEIKTKKTSQIKRLYDIISFFDATNFLKEEFRNAPDKKTFDKDLLFRQYFEERNKAKLLFTLKQGDFVYLPNENEEVILDKESPLYNQYWGDLKERGKNIYVVQKFSKKQIYFIKHTIADIIKKDVEFGSQNCYETVEGRSIKENCFKLEIDRLGNIVKVIKR

SEQ ID NO: 43 - protein, 1338 aa, Coprococcus catus
MKQEYFLGLDMGTGSLGWAVTDSTYQVMRKHGKALWGTRLFESASTAEERRMFRTARRRLDRRNWRIQVLQEIFSEEISKVDPGFFLRMKESKYYPEDKRDAEGNCPELPYALFVDDNYTDKNYHKDYPTIYHLRKMLMETTEIPDIRLVYLVLHHMMKHRGHFLLSGDISQIKEFKSTFEQLIQNIQDEELEWHISLDDAAIQFVEHVLKDRNLTRSTKKSRLIKQLNAKSACEKAILNLLSGGTVKLSDIFNNKELDESERPKVSFADSGYDDYIGIVEAELAEQYYIIASAKAVYDWSVLVEILGNSVSISEAKIKVYQKHQADLKTLKKIVRQYMTKEDYKRVFVDTEEKLNNYSAYIGMTKKNGKKVDLKSKQCTQADFYDFLKKNVIKVIDHKEITQEIESEIEKENFLPKQVTKDNGVIPYQVHDYELKKILDNLGTRMPFIKENAEKIQQLFEFRIPYYVGPLNRVDDGKDGKFTWSVRKSDARIYPWNFTEVIDVEASAEKFIRRMTNKCTYLVGEDVLPKDSLVYSKFMVLNELNNLRLNGEKISVELKQRIYEELFCKYRKVTRKKLERYLVIEGIAKKGVEITGIDGDFKASLTAYHDFKERLTDVQLSQRAKEAIVLNVVLFGDDKKLLKQRLSKMYPNLTTGQLKGICSLSYQGWGRLSKTFLEEITVPAPGTGEVWNIMTALWQTNDNLMQLLSRNYGFTNEVEEFNTLKKETDLSYKTVDELYVSPAVKRQIWQTLKVVKEIQKVMGNAPKRVFVEMAREKQEGKRSDSRKKQLVELYRACKNEERDWITELNAQSDQQLRSDKLFLYYIQKGRCMYSGETIQLDELWDNTKYDIDHIYPQSKTMDDSLNNRVLVKKNYNAIKSDTYPLSLDIQKKMMSFWKMLQQQGFITKEKYVRLVRSDELSADELAGFIERQIVETRQSTKAVATILKEALPDTEIVYVKAGNVSNFRQTYELLKVREMNDLHHAKDAYLNIVVGNAYFVKFTKNAAWFIRNNPGRSYNLKRMFEFDIERSGEIAWKAGNKGSIVTVKKVMQKNNILVTRKAYEVKGGLFDQQIMKKGKGQVPIKGNDERLADIEKYGGYNKAAGTYFMLVKSLDKKGKEIRTIEFVPLYLKNQIEINHESAIQYLAQERGLNSPEILLSKIKIDTLFKVDGFKMWLSGRTGNQLIFKGANQLILSHQEAAILKGVVKYVNRKNENKDAKLSERDGMTEEKLLQLYDTFLDKLSNTVYSIRLSAQIKTLTEKRAKFIGLSNEDQCIVLNEILHMFQCQSGSANLKLIGGPGSAGILVMNNNITACKQISVINQSPTGIYEKEIDLIKL

SEQ ID NO: 44 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 45 - protein, 1195 aa, Elusimicrobium minutum
MQKNINTKQNHIYIKQAQKIKEKLGDKPYRIGLDLGVGSIGFAIVSMEENDGNVLLPKEIIMVGSRIFKASAGAADRKLSRGQRNNHRHTRERMRYLWKVLAEQKLALPVPADLDRKENSSEGETSAKRFLGDVLQKDIYELRVKSLDERLSLQELGYVLYHIAGHRGSSAIRTFENDSEEAQKENTENKKIAGNIKRLMAKKNYRTYGEYLYKEFFENKEKHKREKISNAANNHKFSPTRDLVIKEAEAILKKQAGKDGFHKELTEEYIEKLTKAIGYESEKLIPESGFCPYLKDEKRLPASHKLNEERRLWETLNNARYSDPIVDIVTGEITGYYEKQFTKEQKQKLFDYLLTGSELTPAQTKKLLGLKNTNFEDIILQGRDKKAQKIKGYKLIKLESMPFWARLSEAQQDSFLYDWNSCPDEKLLTEKLSNEYHLTEEEIDNAFNEIVLSSSYAPLGKSAMLIILEKIKNDLSYTEAVEEALKEGKLTKEKQAIKDRLPYYGAVLQESTQKIIAKGFSPQFKDKGYKTPHTNKYELEYGRIANPVVHQTLNELRKLVNEIIDILGKKPCEIGLETARELKKSAEDRSKLSREQNDNESNRNRIYEIYIRPQQQVIITRRENPRNYILKFELLEEQKSQCPFCGGQISPNDIINNQADIEHLFPIAESEDNGRNNLVISHSACNADKAKRSPWAAFASAAKDSKYDYNRILSNVKENIPHKAWRFNQGAFEKFIENKPMAARFKTDNSYISKVAHKYLACLFEKPNIICVKGSLTAQLRMAWGLQGLMIPFAKQLITEKESESFNKDVNSNKKIRLDNRHHALDAIVIAYASRGYGNLLNKMAGKDYKINYSERNWLSKILLPPNNIVWENIDADLESFESSVKTALKNAFISVKHDHSDNGELVKGTMYKIFYSERGYTLTTYKKLSALKLTDPQKKKTPKDFLETALLKFKGRESEMKNEKIKSAIENNKRLFDVIQDNLEKAKKLLEEENEKSKAEGKKEKNINDASIYQKAISLSGDKYVQLSKKEPGKFFAISKPTPTTTGYGYDTGDSLCVDLYYDNKGKLCGEIIRKIDAQQKNPLKYKEQGFTLFERIYGGDILEVDFDIHSDKNSFRNNTGSAPENRVFIKVGTFTEITNNNIQIWFGNIIKSTGGQDDSFTINSMQQYNPRKLILSSCGFIKYRSPILKNKEG

SEQ ID NO: 46 - protein, 1062 aa, Treponema sp.
MIMKLEKWRLGLDLGTNSIGWSVFSLDKDNSVQDLIDMGVRIFSDGRDPKTKEPLAVARRTARSQRKLIYRRKLRRKQVFKFLQEQGLFPKTKEECMTLKSLNPYELRIKALDEKLEPYELGRALFNLAVRRGFKSNRKDGSREEVSEKKSPDEIKTQADMQTHLEKAIKENGCRTITEFLYKNQGENGGIRFAPGRMTYYPTRKMYEEEFNLIRSKQEKYYPQVDWDDIYKAIFYQRPLKPQQRGYCIYENDKERTFKAMPCSQKLRILQDIGNLAYYEGGSKKRVELNDNQDKVLYELLNSKDKVTFDQMRKALCLADSNSFNLEENRDFLIGNPTAVKMRSKNRFGKLWDEIPLEEQDLIIETIITADEDDAVYEVIKKYDLTQEQRDFIVKNTILQSGTSMLCKEVSEKLVKRLEEIADLKYHEAVESLGYKFADQTVEKYDLLPYYGKVLPGSTMEIDLSAPETNPEKHYGKISNPTVHVALNQTRVVVNALIKEYGKPSQIAIELSRDLKNNVEKKAEIARKQNQRAKENIAINDTISALYHTAFPGKSFYPNRNDRMKYRLWSELGLGNKCIYCGKGISGAELFTKEIEIEHILPFSRTLLDAESNLTVAHSSCNAFKAERSPFEAFGTNPSGYSWQEIIQRANQLKNTSKKNKFSPNAMDSFEKDSSFIARQLSDNQYIAKAALRYLKCLVENPSDVWTTNGSMTKLLRDKWEMDSILCRKFTEKEVALLGLKPEQIGNYKKNRFDHRHHAIDAVVIGLTDRSMVQKLATKNSHKGNRIEIPEFPILRSDLIEKVKNIVVSFKPDHGAEGKLSKETLLGKIKLHGKETFVCRENIVSLSEKNLDDIVDEKIKSKVKDYVAKHKGQKIEAVLSDFSKENGIKKVRCVNRVQTPIEITSGKISRYLSPEDYFAAVIWEIPGEKKTFKAQYIRRNEVEKNSKGLNVVKPAVLENGKPHPAAKQVCLLHKDDYLEFSDKGKMYFCRIAGYAATNNKLDIRPVYAVSYCADWINSTNETMLTGYWKPTPTQNWVSVNVLFDKQKARLVTVSPIGRVFRK

SEQ ID NO: 47 - protein, 1054 aa, Staphylococcus lugdunensis
MNQKFILGLDIGITSVGYGLIDYETKNIIDAGVRLFPEANVENNEGRRSKRGSRRLKRRRIHRLERVKKLLEDYNLLDQSQIPQSTNPYAIRVKGLSEALSKDELVIALLHIAKRRGIHKIDVIDSNDDVGNELSTKEQLNKNSKLLKDKFVCQIQLERMNEGQVRGEKNRFKTADIIKEIIQLLNVQKNFHQLDENFINKYIELVEMRREYFEGPGKGSPYGWEGDPKAWYETLMGHCTYFPDELRSVKYAYSADLFNALNDLNNLVIQRDGLSKLEYHEKYHIIENVFKQKKKPTLKQIANEINVNPEDIKGYRITKSGKPQFTEFKLYHDLKSVLFDQSILENEDVLDQIAEILTIYQDKDSIKSKLTELDILLNEEDKENIAQLTGYTGTHRLSLKCIRLVLEEQWYSSRNQMEIFTHLNIKPKKINLTAANKIPKAMIDEFILSPVVKRTFGQAINLINKIIEKYGVPEDIIIELARENNSKDKQKFINEMQKKNENTRKRINEIIGKYGNQNAKRLVEKIRLHDEQEGKCLYSLESIPLEDLLNNPNHYEVDHIIPRSVSFDNSYHNKVLVKQSENSKKSNLTPYQYFNSGKSKLSYNQFKQHILNLSKSQDRISKKKKEYLLEERDINKFEVQKEFINRNLVDTRYATRELTNYLKAYFSANNMNVKVKTINGSFTDYLRKVWKFKKERNHGYKHHAEDALIIANADFLFKENKKLKAVNSVLEKPEIESKQLDIQVDSEDNYSEMFIIPKQVQDIKDFRNFKYSHRVDKKPNRQLINDTLYSTRKKDNSTYIVQTIKDIYAKDNTTLKKQFDKSPEKFLMYQHDPRTFEKLEVIMKQYANEKNPLAKYHEETGEYLTKYSKKNNGPIVKSLKYIGNKLGSHLDVTHQFKSSTKKLVKLSIKPYRFDVYLTDKGYKFITISYLDVLKKDNYYYIPEQKYDKLKLGKAIDKNAKFIASFYKNDLIKLDGEIYKIIGVNSDTRNMIELDLPDIRYKEYCELNNIKGEPRIKKTIGKKVNSIEKLTTDVLGNVFTNTQYTKPQLLFKRGN

SEQ ID NO: 48 - protein, 1388 aa, Streptococcus thermophilus
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 49 - protein, 1436 aa, Bacteroides fragilis
MKRILGLDLGTNSIGWALVNEAENKDERSSIVKLGVRVNPLTVDELTNFEKGKSITTNADRTLKRGMRRNLQRYKLRRETLTEVLKEHKLITEDTILSENGNRTTFETYRLRAKAVTEEISLEEFARVLLMINKKRGYKSSRKAKGVEEGTLIDGMDIARELYNNNLTPGELCLQLLDAGKKFLPDFYRSDLQNELDRIWEKQKEYYPEILTDVLKEELRGKKRDAVWAICAKYFVWKENYTEWNKEKGKTEQQEREHKLEGIYSKRKRDEAKRENLQWRVNGLKEKLSLEQLVIVFQEMNTQINNSSGYLGAISDRSKELYFNKQTVGQYQMEMLDKNPNASLRNMVFYRQDYLDEFNMLWEKQAVYHKELTEELKKEIRDIIIFYQRRLKSQKGLIGFCEFESRQIEVDIDGKKKIKTVGNRVISRSSPLFQEFKIWQILNNIEVTVVGKKRKRRKLKENYSALFEELNDAEQLELNGSRRLCQEEKELLAQELFIRDKMTKSEVLKLLFDNPQELDLNFKTIDGNKTGYALFQAYSKMIEMSGHEPVDFKKPVEKVVEYIKAVFDLLNWNTDILGFNSNEELDNQPYYKLWHLLYSFEGDNTPTGNGRLIQKMTELYGFEKEYATILANVSFQDDYGSLSAKAIHKILPHLKEGNRYDVACVYAGYRHSESSLTREEIANKVLKDRLMLLPKNSLHNPVVEKILNQMVNVINVIIDIYGKPDEIRVELARELKKNAKEREELTKSIAQTTKAHEEYKTLLQTEFGLTNVSRTDILRYKLYKELESCGYKTLYSNTYISREKLFSKEFDIEHIIPQARLFDDSFSNKTLEARSVNIEKGNKTAYDFVKEKFGESGADNSLEHYLNNIEDLFKSGKISKTKYNKLKMAEQDIPDGFIERDLRNTQYIAKKALSMLNEISHRVVATSGSVTDKLREDWQLIDVMKELNWEKYKALGLVEYFEDRDGRQIGRIKDWTKRNDHRHHAMDALTVAFTKDVFIQYFNNKNASLDPNANEHAIKNKYFQNGRAIAPMPLREFRAEAKKHLENTLISIKAKNKVITGNINKTRKKGGVNKNMQQTPRGQLHLETIYGSGKQYLTKEEKVNASFDMRKIGTVSKSAYRDALLKRLYENDNDPKKAFAGKNSLDKQPIWLDKEQMRKVPEKVKIVTLEAIYTIRKEISPDLKVDKVIDVGVRKILIDRLNEYGNDAKKAFSNLDKNPIWLNKEKGISIKRVTISGISNAQSLHVKKDKDGKPILDENGRNIPVDFVNTGNNHHVAVYYRPVIDKRGQLVVDEAGNPKYELEEVVVSFFEAVTRANLGLPIIDKDYKTTEGWQFLFSMKQNEYFVFPNEKTGFNPKEIDLLDVENYGLISPNLFRVQKFSLKNYVFRHHLETTIKDTSSILRGITWIDFRSSKGLDTIVKVRVNHIGQIVSVGEY

SEQ ID NO: 50 - protein, 1398 aa, Veillonella atypica
METQTSNQLITSHLKDYPKQDYFVGLDIGTNSVGWAVTNTSYELLKFHSHKMWGSRLFEEGESAVTRRGFRSMRRRLERRKLRLKLLEELFADAMAQVDSTFFIRLHESKYHYEDKTTGHSSKHILFIDEDYTDQDYFTEYPTIYHLRKDLMENGTDDIRKLFLAVHHILKYRGNFLYEGATFNSNAFTFEDVLKQALVNITFNCFDTNSAISSISNILMESGKTKSDKAKAIERLVDTYTVFDEVNTPDKPQKEQVKEDKKTLKAFANLVLGLSANLIDLFGSVEDIDDDLKKLQIVGDTYDEKRDELAKVWGDEIHIIDDCKSVYDAIILMSIKEPGLTISQSKVKAFDKHKEDLVILKSLLKLDRNVYNEMFKSDKKGLHNYVHYIKQGRTEETSCSREDFYKYTKKIVEGLADSKDKEYILNEIELQTLLPLQRIKDNGVIPYQLHLEELKVILDKCGPKFPFLHTVSDGFSVTEKLIKMLEFRIPYYVGPLNTHHNIDNGGFSWAVRKQAGRVTPWNFEEKIDREKSAAAFIKNLTNKCTYLFGEDVLPKSSLLYSEFMLLNELNNVRIDGKALAQGVKQHLIDSIFKQDHKKMTKNRIELFLKDNNYITKKHKPEITGLDGEIKNDLTSYRDMVRILGNNFDVSMAEDIITDITIFGESKKMLRQTLRNKFGSQLNDETIKKLSKLRYRDWGRLSKKLLKGIDGCDKAGNGAPKTIIELMRNDSYNLMEILGDKFSFMECIEEENAKLAQGQVVNPHDIIDELALSPAVKRAVWQALRIVDEVAHIKKALPSRIFVEVARTNKSEKKKKDSRQKRLSDLYSAIKKDDVLQSGLQDKEFGALKSGLANYDDAALRSKKLYLYYTQMGRCAYTGNIIDLNQLNTDNYDIDHIYPRSLTKDDSFDNLVLCERTANAKKSDIYPIDNRIQTKQKPFWAFLKHQGLISERKYERLTRIAPLTADDLSGFIARQLVETNQSVKATTTLLRRLYPDIDVVFVKAENVSDFRHNNNFIKVRSLNHHHHAKDAYLNIVVGNVYHEKFTRNFRLFFKKNGANRTYNLAKMFNYDVICTNAQDGKAWDVKTSMNTVKKMMASNDVRVTRRLLEQSGALADATIYKASVAAKAKDGAYIGMKTKYSVFADVTKYGGMTKIKNAYSIIVQYTGKKGEEIKEIVPLPIYLINRNATDIELIDYVKSVIPKAKDISIKYRKLCINQLVKVNGFYYYLGGKTNDKIYIDNAIELVVPHDIATYIKLLDKYDLLRKENKTLKASSITTSIYNINTSTVVSLSNKVGIDVFDYFMSKLRTPLYMKMKGNKVDELSSTGRSKFIKMTLEEQSIYLLEVLNLLTNSKTTFDVKPLGITGSRSTIGVKIHNLDEFKIINESITGLYSNEVTIV

SEQ ID NO: 51 - protein, 1092 aa, Ilyobacter polytropus
MKYSIGLDIGIASVGWSVINKDKERIEDMGVRIFQKAENPKDGSSLASSRREKRGSRRRNRRKKHRLDRIKNILCESGLVKKNEIEKIYKNAYLKSPWELRAKSLEAKISNKEIAQILLHIAKRRGFKSFRKTDRNADDTGKLLSGIQENKKIMEEKGYLTIGDMVAKDPKFNTHVRNKAGSYLFSFSRKLLEDEVRKIQAKQKELGNTHFTDDVLEKYIEVFNSQRNFDEGPSKPSPYYSEIGQIAKMIGNCTFESSEKRTAKNTWSGERFVFLQKLNNFRIVGLSGKRPLTEEERDIVEKEVYLKKEVRYEKLRKILYLKEEERFGDLNYSKDEKQDKKTEKTKFISLIGNYTIKKLNLSEKLKSEIEEDKSKLDKIIEILTFNKSDKTIESNLKKLELSREDIEILLSEEFSGTLNLSLKAIKKILPYLEKGLSYNEACEKADYDYKNNGIKFKRGELLPVVDKDLIANPVVLRAISQTRKVVNAIIRKYGTPHTIHVEVARDLAKSYDDRQTIIKENKKRELENEKTKKFISEEFGIKNVKGKLLLKYRLYQEQEGRCAYSRKELSLSEVILDESMTDIDHIIPYSRSMDDSYSNKVLVLSGENRKKSNLLPKEYFDRQGRDWDTFVLNVKAMKIHPRKKSNLLKEKFTREDNKDWKSRALNDTRYISRFVANYLENALEYRDDSPKKRVFMIPGQLTAQLRARWRLNKVRENGDLHHALDAAVVAVTDQKAINNISNISRYKELKNCKDVIPSIEYHADEETGEVYFEEVKDTRFPMPWSGFDLELQKRLESENPREEFYNLLSDKRYLGWFNYEEGFIEKLRPVFVSRMPNRGVKGQAHQETIRSSKKISNQIAVSKKPLNSIKLKDLEKMQGRDTDRKLYEALKNRLEEYDDKPEKAFAEPFYKPTNSGKRGPLVRGIKVEEKQNVGVYVNGGQASNGSMVRIDVFRKNGKFYTVPIYVHQTLLKELPNRAINGKPYKDWDLIDGSFEFLYSFYPNDLIEIEFGKSKSIKNDNKLTKTEIPEVNLSEVLGYYRGMDTSTGAATIDTQDGKIQMRIGIKTVKNIKKYQVDVLGNVYKVKREKRQTF

SEQ ID NO: 52 - protein, 1517 aa, Parabacteroides sp.
MKKIVGLDLGTNSIGWALINAYINKEHLYGIEACGSRIIPMDAAILGNFDKGNSISQTADRTSYRGIRRLRERHLLRRERLHRILDLLGFLPKHYSDSLNRYGKFLNDIECKLPWVKDETGSYKFIFQESFKEMLANFTEHHPILIANNKKVPYDWTIYYLRKKALTQKISKEELAWILLNFNQKRGYYQLRGEEEETPNKLVEYYSLKVEKVEDSGERKGKDTWYNVHLENGMIYRRTSNIPLDWEGKTKEFIVTTDLEADGSPKKDKEGNIKRSFRAPKDDDWTLIKKKTEADIDKIKMTVGAYIYDTLLQKPDQKIRGKLVRTIERKYYKNELYQILKTQSEFHEELRDKQLYIACLNELYPNNEPRRNSISTRDFCHLFIEDIIFYQRPLKSKKSLIDNCPYEENRYIDKESGEIKHASIKCIAKSHPLYQEFRLWQFIVNLRIYRKETDVDVTQELLPTEADYVTLFEWLNEKKEIDQKAFFKYPPFGFKKTTSNYRWNYVEDKPYPCNETHAQIIARLGKAHIPKAFLSKEKEETLWHILYSIEDKQEIEKALHSFANKNNLSEEFIEQFKNFPPFKKEYGSYSAKAIKKLLPLMRMGKYWSIENIDNGTRIRINKIIDGEYDENIRERVRQKAINLTDITHFRALPLWLACYLVYDRHSEVKDIVKWKTPKDIDLYLKSFKQHSLRNPIVEQVITETLRTVRDIWQQVGHIDEIHIELGREMKNPADKRARMSQQMIKNENTNLRIKALLTEFLNPEFGIENVRPYSPSQQDLLRIYEEGVLNSILELPEDIGIILGKFNQTDTLKRPTRSEILRYKLWLEQKYRSPYTGEMIPLSKLFTPAYEIEHIIPQSRYFDDSLSNKVICESEINKLKDRSLGYEFIKNHHGEKVELAFDKPVEVLSVEAYEKLVHESYSHNRSKMKKLLMEDIPDQFIERQLNDSRYISKVVKSLLSNIVREENEQEAISKNVIPCTGGITDRLKKDWGINDVWNKIVLPRFIRLNELTESTRFTSINTNNTMIPSMPLELQKGFNKKRIDHRHHAMDAIIIACANRNIVNYLNNVSASKNTKITRRDLQTLLCHKDKTDNNGNYKWVIDKPWETFTQDTLTALQKITVSFKQNLRVINKTTNHYQHYENGKKIVSNQSKGDSWAIRKSMHKETVHGEVNLRMIKTVSFNEALKKPQAIVEMDLKKKILAMLELGYDTKRIKNYFEENKDTWQDINPSKIKVYYFTKETKDRYFAVRKPIDTSFDKKKIKESITDTGIQQIMLRHLETKDNDPTLAFSPDGIDEMNRNILILNKGKKHQPIYKVRVYEKAEKFTVGQKGNKRTKFVEAAKGTNLFFAIYETEEIDKDTKKVIRKRSYSTIPLNVVIERQKQGLSSAPEDENGNLPKYILSPNDLVYVPTQEEINKGEVVMPIDRDRIYKMVDSSGITANFIPASTANLIFALPKATAEIYCNGENCIQNEYGIGSPQSKNQKAITGEMVKEICFPIKVDRLGNIIQVGSCILTN

SEQ ID NO: 53 - protein, 1323 aa, Fructobacillus fructosus
MVYDVGLDIGTGSVGWVALDENGKLARAKGKNLVGVRLFDTAQTAADRRGFRTTRRRLSRRKWRLRLLDELFSAEINEIDSSFFQRLKYSYVHPKDEENKAHYYGGYLFPTEEETKKFHRSYPTIYHLRQELMAQPNKRFDIREIYLAIHHLVKYRGHFLSSQEKITIGSTYNPEDLANAIEVYADEKGLSWELNNPEQLTEIISGEAGYGLNKSMKADEALKLFEFDNNQDKVAIKTLLAGLTGNQIDFAKLFGKDISDKDEAKLWKLKLDDEALEEKSQTILSQLTDEEIELFHAVVQAYDGFVLIGLLNGADSVSAAMVQLYDQHREDRKLLKSLAQKAGLKHKRFSEIYEQLALATDEATIKNGISTARELVEESNLSKEVKEDTLRRLDENEFLPKQRTKANSVIPHQLHLAELQKILQNQGQYYPFLLDTFEKEDGQDNKIEELLRFRIPYYVGPLVTKKDVEHAGGDADNHWVERNEGFEKSRVTPWNFDKVFNRDKAARDFIERLTGNDTYLIGEKTLPQNSLRYQLFTVLNELNNVRVNGKKFDSKTKADLINDLFKARKTVSLSALKDYLKAQGKGDVTITGLADESKFNSSLSSYNDLKKTFDAEYLENEDNQETLEKIIEIQTVFEDSKIASRELSKLPLDDDQVKKLSQTHYTGWGRLSEKLLDSKIIDERGQKVSILDKLKSTSQNFMSIINNDKYGVQAWITEQNTGSSKLTFDEKVNELTTSPANKRGIKQSFAVLNDIKKAMKEEPRRVYLEFAREDQTSVRSVPRYNQLKEKYQSKSLSEEAKVLKKTLDGNKNKMSDDRYFLYFQQQGKDMYTGRPINFERLSQDYDIDHIIPQAFTKDDSLDNRVLVSRPENARKSDSFAYTDEVQKQDGSLWTSLLKSGFINRKKYERLTKAGKYLDGQKTGFIARQLVETRQIIKNVASLIEGEYENSKAVAIRSEITADMRLLVGIKKHREINSFHHAFDALLITAAGQYMQNRYPDRDSTNVYNEFDRYTNDYLKNLRQLSSRDEVRRLKSFGFVVGTMRKGNEDWSEENTSYLRKVMMFKNILTTKKTEKDRGPLNKETIFSPKSGKKLIPLNSKRSDTALYGGYSNVYSAYMTLVRANGKNLLIKIPISIANQIEVGNLKINDYIVNNPAIKKFEKILISKLPLGQLVNEDGNLIYLASNEYRHNAKQLWLSTTDADKIASISENSSDEELLEAYDILTSENVKNRFPFFKKDIDKLSQVRDEFLDSDKRIAVIQTILRGLQIDAAYQAPVKIISKKVSDWHKLQQSGGIKLSDNSEMIYQSATGIFETRVKISDLL

SEQ ID NO: 54 - protein, 1088 aa, Bacillus smithii
MNYKMGLDIGIASVGWAVINLDLKRIEDLGVRIFDKAEHPQNGESLALPRRIARSARRRLRRRKHRLERIRRLLVSENVLTKEEMNLLFKQKKQIDVWQLRVDALERKLNNDELARVLLHLAKRRGFKSNRKSERNSKESSEFLKNIEENQSILAQYRSVGEMIVKDSKFAYHKRNKLDSYSNMIARDDLEREIKLIFEKQREFNNPVCTERLEEKYLNIWSSQRPFASKEDIEKKVGFCTFEPKEKRAPKATYTFQSFIVWEHINKLRLVSPDETRALTEIERNLLYKQAFSKNKMTYYDIRKLLNLSDDIHFKGLLYDPKSSLKQIENIRFLELDSYHKIRKCIENVYGKDGIRMFNETDIDTFGYALTIFKDDEDIVAYLQNEYITKNGKRVSNLANKVYDKSLIDELLNLSFSKFAHLSMKAIRNILPYMEQGEIYSKACELAGYNFTGPKKKEKALLLPVIPNIANPVVMRALTQSRKVVNAIIKKYGSPVSIHIELARDLSHSFDERKKIQKDQTENRKKNETAIKQLIEYELTKNPTGLDIVKFKLWSEQQGRCMYSLKPIELERLLEPGYVEVDHILPYSRSLDDSYANKVLVLTKENREKGNHTPVEYLGLGSERWKKFEKFVLANKQFSKKKKQNLLRLRYEETEEKEFKERNLNDTRYISKFFANFIKEHLKFADGDGGQKVYTINGKITAHLRSRWDFNKNREESDLHHAVDAVIVACATQGMIKKITEFYKAREQNKESAKKKEPIFPQPWPHFADELKARLSKFPQESIEAFALGNYDRKKLESLRPVFVSRMPKRSVTGAAHQETLRRCVGIDEQSGKIQTAVKTKLSDIKLDKDGHFPMYQKESDPRTYEAIRQRLLEHNNDPKKAFQEPLYKPKKNGEPGPVIRTVKIIDTKNKVVHLDGSKTVAYNSNIVRTDVFEKDGKYYCVPVYTMDIMKGTLPNKAIEANKPYSEWKEMTEEYTFQFSLFPNDLVRIVLPREKTIKTSTNEEIIIKDIFAYYKTIDSATGGLELISHDRNFSLRGVGSKTLKRFEKYQVDVLGNIHKVKGEKRVGLAAPTNQKKGKTVDSLQSVSD

SEQ ID NO: 55 - protein, 1233 aa, Mycoplasma canis
MEKKRKVTLGFDLGIASVGWAIVDSETNQVYKLGSRLFDAPDTNLERRTQRGTRRLLRRRKYRNQKFYNLVKRTEVFGLSSREAIENRFRELSIKYPNIIELKTKALSQEVCPDEIAWILHDYLKNRGYFYDEKETKEDFDQQTVESMPSYKLNEFYKKYGYFKGALSQPTESEMKDNKDLKEAFFFDFSNKEWLKEINYFFNVQKNILSETFIEEFKKIFSFTRDISKGPGSDNMPSPYGIFGEFGDNGQGGRYEHIWDKNIGKCSIFTNEQRAPKYLPSALIFNFLNELANIRLYSTDKKNIQPLWKLSSVDKLNILLNLFNLPISEKKKKLTSTNINDIVKKESIKSIMISVEDIDMIKDEWAGKEPNVYGVGLSGLNIEESAKENKFKFQDLKILNVLINLLDNVGIKFEFKDRNDIIKNLELLDNLYLFLIYQKESNNKDSSIDLFIAKNESLNIENLKLKLKEFLLGAGNEFENHNSKTHSLSKKAIDEILPKLLDNNEGWNLEAIKNYDEEIKSQIEDNSSLMAKQDKKYLNDNFLKDAILPPNVKVTFQQAILIFNKIIQKFSKDFEIDKVVIELAREMTQDQENDALKGIAKAQKSKKSLVEERLEANNIDKSVFNDKYEKLIYKIFLWISQDFKDPYTGAQISVNEIVNNKVEIDHIIPYSLCFDDSSANKVLVHKQSNQEKSNSLPYEYIKQGHSGWNWDEFTKYVKRVFVNNVDSILSKKERLKKSENLLTASYDGYDKLGFLARNLNDTRYATILFRDQLNNYAEHHLIDNKKMFKVIAMNGAVTSFIRKNMSYDNKLRLKDRSDFSHHAYDAAIIALFSNKTKTLYNLIDPSLNGIISKRSEGYWVIEDRYTGEIKELKKEDWTSIKNNVQARKIAKEIEEYLIDLDDEVFFSRKTKRKTNRQLYNETIYGIATKTDEDGITNYYKKEKFSILDDKDIYLRLLREREKFVINQSNPEVIDQIIEIIESYGKENNIPSRDEAINIKYTKNKINYNLYLKQYMRSLTKSLDQFSEEFINQMIANKTFVLYNPTKNTTRKIKFLRLVNDVKINDIRKNQVINKFNGKNNEPKAFYENINSLGAIVFKNSANNFKTLSINTQIAIFGDKNWDIEDFKTYNMEKIEKYKEIYGIDKTYNFHSFIFPGTILLDKQNKEFYYISSIQTVRDIIEIKFLNKIEFKDENKNQDTSKTPKRLMFGIKSIMNNYEQVDISPFGINKKIFE

SEQ ID NO: 56 - protein, 1498 aa, Odoribacter laneus
METTLGIDLGTNSIGLALVDQEEHQILYSGVRIFPEGINKDTIGLGEKEESRNATRRAKRQMRRQYFRKKLRKAKLLELLIAYDMCPLKPEDVRRWKNWDKQQKSTVRQFPDTPAFREWLKQNPYELRKQAVTEDVTRPELGRILYQMIQRRGFLSSRKGKEEGKIFTGKDRMVGIDETRKNLQKQTLGAYLYDIAPKNGEKYRFRTERVRARYTLRDMYIREFEIIWQRQAGHLGLAHEQATRKKNIFLEGSATNVRNSKLITHLQAKYGRGHVLIEDTRITVTFQLPLKEVLGGKIEIEEEQLKFKSNESVLFWQRPLRSQKSLLSKCVFEGRNFYDPVHQKWIIAGPTPAPLSHPEFEEFRAYQFINNIIYGKNEHLTAIQREAVFELMCTESKDFNFEKIPKHLKLFEKFNFDDTTKVPACTTISQLRKLFPHPVWEEKREEIWHCFYFYDDNTLLFEKLQKDYALQTNDLEKIKKIRLSESYGNVSLKAIRRINPYLKKGYAYSTAVLLGGIRNSFGKRFEYFKEYEPEIEKAVCRILKEKNAEGEVIRKIKDYLVHNRFGFAKNDRAFQKLYHHSQAITTQAQKERLPETGNLRNPIVQQGLNELRRTVNKLLATCREKYGPSFKFDHIHVEMGRELRSSKTEREKQSRQIRENEKKNEAAKVKLAEYGLKAYRDNIQKYLLYKEIEEKGGTVCCPYTGKTLNISHTLGSDNSVQIEHIIPYSISLDDSLANKTLCDATFNREKGELTPYDFYQKDPSPEKWGASSWEEIEDRAFRLLPYAKAQRFIRRKPQESNEFISRQLNDTRYISKKAVEYLSAICSDVKAFPGQLTAELRHLWGLNNILQSAPDITFPLPVSATENHREYYVITNEQNEVIRLFPKQGETPRTEKGELLLTGEVERKVFRCKGMQEFQTDVSDGKYWRRIKLSSSVTWSPLFAPKPISADGQIVLKGRIEKGVFVCNQLKQKLKTGLPDGSYWISLPVISQTFKEGESVNNSKLTSQQVQLFGRVREGIFRCHNYQCPASGADGNFWCTLDTDTAQPAFTPIKNAPPGVGGGQIILTGDVDDKGIFHADDDLHYELPASLPKGKYYGIFTVESCDPTLIPIELSAPKTSKGENLIEGNIWVDEHTGEVRFDPKKNREDQRHHAIDAIVIALSSQSLFQRLSTYNARRENKKRGLDSTEHFPSPWPGFAQDVRQSVVPLLVSYKQNPKTLCKISKTLYKDGKKIHSCGNAVRGQLHKETVYGQRTAPGATEKSYHIRKDIRELKTSKHIGKVVDITIRQMLLKHLQENYHIDITQEFNIPSNAFFKEGVYRIFLPNKHGEPVPIKKIRMKEELGNAERLKDNINQYVNPRNNHHVMIYQDADGNLKEEIVSFWSVIERQNQGQPIYQLPREGRNIVSILQINDTFLIGLKEEEPEVYRNDLSTLSKHLYRVQKLSGMYYTFRHHLASTLNNEREEFRIQSLEAWKRANPVKVQIDEIGRITFLNGPLC

SEQ ID NO: 57 - protein, 1101 aa, Akkermansia muciniphila
MSRSLTFSFDIGYASIGWAVIASASHDDADPSVCGCGTVLFPKDDCQAFKRREYRRLRRNIRSRRVRIERIGRLLVQAQIITPEMKETSGHPAPFYLASEALKGHRTLAPIELWHVLRWYAHNRGYDNNASWSNSLSEDGGNGEDTERVKHAQDLMDKHGTATMAETICRELKLEEGKADAPMEVSTPAYKNLNTAFPRLIVEKEVRRILELSAPLIPGLTAEIIELIAQHHPLTTEQRGVLLQHGIKLARRYRGSLLFGQLIPRFDNRIISRCPVTWAQVYEAELKKGNSEQSARERAEKLSKVPTANCPEFYEYRMARILCNIRADGEPLSAEIRRELMNQARQEGKLTKASLEKAISSRLGKETETNVSNYFTLHPDSEEALYLNPAVEVLQRSGIGQILSPSVYRIAANRLRRGKSVTPNYLLNLLKSRGESGEALEKKIEKESKKKEADYADTPLKPKYATGRAPYARTVLKKVVEEILDGEDPTRPARGEAHPDGELKAHDGCLYCLLDTDSSVNQHQKERRLDTMTNNHLVRHRMLILDRLLKDLIQDFADGQKDRISRVCVEVGKELTTFSAMDSKKIQRELTLRQKSHTDAVNRLKRKLPGKALSANLIRKCRIAMDMNWTCPFTGATYGDHELENLELEHIVPHSFRQSNALSSLVLTWPGVNRMKGQRTGYDFVEQEQENPVPDKPNLHICSLNNYRELVEKLDDKKGHEDDRRRKKKRKALLMVRGLSHKHQSQNHEAMKEIGMTEGMMTQSSHLMKLACKSIKTSLPDAHIDMIPGAVTAEVRKAWDVFGVFKELCPEAADPDSGKILKENLRSLTHLHHALDACVLGLIPYIIPAHHNGLLRRVLAMRRIPEKLIPQVRPVANQRHYVLNDDGRMMLRDLSASLKENIREQLMEQRVIQHVPADMGGALLKETMQRVLSVDGSGEDAMVSLSKKKDGKKEKNQVKASKLVGVFPEGPSKLKALKAAIEIDGNYGVALDPKPVVIRHIKVFKRIMALKEQNGGKPVRILKKGMLIHLTSSKDPKHAGVWRIESIQDSKGGVKLDLQRAHCAVPKNKTHECNWREVDLISLLKKYQMKRYPTSYTGTPR

SEQ ID NO: 58 - protein, 1079 aa, Dinoroseobacter shibae
MRLGLDIGTSSIGWWLYETDGAGSDARITGVVDGGVRIFSDGRDPKSGASLAVDRRAARAMRRRRDRYLRRRATLMKVLAETGLMPADPAEAKALEALDPFALRAAGLDEPLPLPHLGRALFHLNQRRGFKSNRKTDRGDNESGKIKDATARLDMEMMANGARTYGEFLHKRRQKATDPRHVPSVRTRLSIANRGGPDGKEEAGYDFYPDRRHLEEEFHKLWAAQGAHHPELTETLRDLLFEKIFFQRPLKEPEVGLCLFSGHHGVPPKDPRLPKAHPLTQRRVLYETVNQLRVTADGREARPLTREERDQVIHALDNKKPTKSLSSMVLKLPALAKVLKLRDGERFTLETGVRDAIACDPLRASPAHPDRFGPRWSILDADAQWEVISRIRRVQSDAEHAALVDWLTEAHGLDRAHAEATAHAPLPDGYGRLGLTATTRILYQLTADVVTYADAVKACGWHHSDGRTGECFDRLPYYGEVLERHVIPGSYHPDDDDITRFGRITNPTVHIGLNQLRRLVNRIIETHGKPHQIVVELARDLKKSEEQKRADIKRIRDTTEAAKKRSEKLEELEIEDNGRNRMLLRLWEDLNPDDAMRRFCPYTGTRISAAMIFDGSCDVDHILPYSRTLDDSFPNRTLCLREANRQKRNQTPWQAWGDTPHWHAIAANLKNLPENKRWRFAPDAMTRFEGENGFLDRALKDTQYLARISRSYLDTLFTKGGHVWVVPGRFTEMLRRHWGLNSLLSDAGRGAVKAKNRTDHRHHAIDAAVIAATDPGLLNRISRAAGQGEAAGQSAELIARDTPPPWEGFRDDLRVRLDRIIVSHRADHGRIDHAARKQGRDSTAGQLHQETAYSIVDDIHVASRTDLLSLKPAQLLDEPGRSGQVRDPQLRKALRVATGGKTGKDFENALRYFASKPGPYQAIRRVRIIKPLQAQARVPVPAQDPIKAYQGGSNHLFEIWRLPDGEIEAQVITSFEAHTLEGEKRPHPAAKRLLRVHKGDMVALERDGRRVVGHVQKMDIANGLFIVPHNEANADTRNNDKSDPFKWIQIGARPAIASGIRRVSVDEIGRLRDGGTRPI

SEQ ID NO: 59 - protein, 1059 aa, Wolinella succinogenes
MIERILGVDLGISSLGWAIVEYDKDDEAANRIIDCGVRLFTAAETPKKKESPNKARREARGIRRVLNRRRVRMNMIKKLFLRAGLIQDVDLDGEGGMFYSKANRADVWELRHDGLYRLLKGDELARVLIHIAKHRGYKFIGDDEADEESGKVKKAGVVLRQNFEAAGCRTVGEWLWRERGANGKKRNKHGDYEISIHRDLLVEEVEAIFVAQQEMRSTIATDALKAAYREIAFFVRPMQRIEKMVGHCTYFPEERRAPKSAPTAEKFIAISKFFSTVIIDNEGWEQKIIERKTLEELLDFAVSREKVEFRHLRKFLDLSDNEIFKGLHYKGKPKTAKKREATLFDPNEPTELEFDKVEAEKKAWISLRGAAKLREALGNEFYGRFVALGKHADEATKILTYYKDEGQKRRELTKLPLEAEMVERLVKIGFSDFLKLSLKAIRDILPAMESGARYDEAVLMLGVPHKEKSAILPPLNKTDIDILNPTVIRAFAQFRKVANALVRKYGAFDRVHFELAREINTKGEIEDIKESQRKNEKERKEAADWIAETSFQVPLTRKNILKKRLYIQQDGRCAYTGDVIELERLFDEGYCEIDHILPRSRSADDSFANKVLCLARANQQKTDRTPYEWFGHDAARWNAFETRTSAPSNRVRTGKGKIDRLLKKNFDENSEMAFKDRNLNDTRYMARAIKTYCEQYWVFKNSHTKAPVQVRSGKLTSVLRYQWGLESKDRESHTHHAVDAIIIAFSTQGMVQKLSEYYRFKETHREKERPKLAVPLANFRDAVEEATRIENTETVKEGVEVKRLLISRPPRARVTGQAHEQTAKPYPRIKQVKNKKKWRLAPIDEEKFESFKADRVASANQKNFYETSTIPRVDVYHKKGKFHLVPIYLHEMVLNELPNLSLGTNPEAMDENFFKFSIFKDDLISIQTQGTPKKPAKIIMGYFKNMHGANMVLSSINNSPCEGFTCTPVSMDKKHKDKCKLCPEENRIAGRCLQGFLDYWSQEGLRPPRKEFECDQGVKFALDVKKYQIDPLGYYYEVKQEKRLGTIPQMRSAKKLVKK

SEQ ID NO: 60 - protein, 1428 aa, Parasutterella excrementihominis
MGKTHIIGVGLDLGGTYTGTFITSHPSDEAEHRDHSSAFTVVNSEKLSFSSKSRTAVRHRVRSYKGFDLRRRLLLLVAEYQLLQKKQTLAPEERENLRIALSGYLKRRGYARTEAETDTSVLESLDPSVFSSAPSFTNFFNDSEPLNIQWEAIANSPETTKALNKELSGQKEADFKKYIKTSFPEYSAKEILANYVEGRRAILDASKYIANLQSLGHKHRSKYLSDILQDMKRDSRITRLSEAFGSTDNLWRIIGNISNLQERAVRWYFNDAKFEQGQEQLDAVKLKNVLVRALKYLRSDDKEWSASQKQIIQSLEQSGDVLDVLAGLDPDRTIPPYEDQNNRRPPEDQTLYLNPKALSSEYGEKWKSWANKFAGAYPLLTEDLTEILKNTDRKSRIKIRSDVLPDSDYRLAYILQRAFDRSIALDECSIRRTAEDFENGVVIKNEKLEDVLSGHQLEEFLEFANRYYQETAKAKNGLWFPENALLERADLHPPMKNKILNVIVGQALGVSPAEGTDFIEEIWNSKVKGRSTVRSICNAIENERKTYGPYFSEDYKFVKTALKEGKTEKELSKKFAAVIKVLKMVSEVVPFIGKELRLSDEAQSKFDNLYSLAQLYNLIETERNGFSKVSLAAHLENAWRMTMTDGSAQCCRLPADCVRPFDGFIRKAIDRNSWEVAKRIAEEVKKSVDFTNGTVKIPVAIEANSFNFTASLTDLKYIQLKEQKLKKKLEDIQRNEENQEKRWLSKEERIRADSHGICAYTGRPLDDVGEIDHIIPRSLTLKKSESIYNSEVNLIFVSAQGNQEKKNNIYLLSNLAKNYLAAVFGTSDLSQITNEIESTVLQLKAAGRLGYFDLLSEKERACARHALFLNSDSEARRAVIDVLGSRRKASVNGTQAWFVRSIFSKVRQALAAWTQETGNELIFDAISVPAADSSEMRKRFAEYRPEFRKPKVQPVASHSIDAMCIYLAACSDPFKTKRMGSQLAIYEPINFDNLFTGSCQVIQNTPRNFSDKTNIANSPIFKETIYAERFLDIIVSRGEIFIGYPSNMPFEEKPNRISIGGKDPFSILSVLGAYLDKAPSSEKEKLTIYRVVKNKAFELFSKVAGSKFTAEEDKAAKILEALHFVTVKQDVAATVSDLIKSKKELSKDSIENLAKQKGCLKKVEYSSKEFKFKGSLIIPAAVEWGKVLWNVFKENTAEELKDENALRKALEAAWPSSFGTRNLHSKAKRVFSLPVVATQSGAVRIRRKTAFGDFVYQSQDTNNLYSSFPVKNGKLDWSSPIIHPALQNRNLTAYGYRFVDHDRSISMSEFREVYNKDDLMRIELAQGTSSRRYLRVEMPGEKFLAWFGENSISLGSSFKFSVSEVFDNKIYTENAEFTKFLPKPREDNKHNGTIFFELVGPRVIFNYIVGGAASSLKEIFSEAGKERS

SEQ ID NO: 61 - protein, 1421 aa, Streptococcus sanguinis
MTKFNKNYSIGLDIGVSSVGYAVVTEDYRVPAFKFKVLGNTEKEKIKKNLIGSTTFVSAQPAKGTRVFRVNRRRIDRRNHRITYLRDIFQKEIEKVDKNFYRRLDESFRVLGDKSEDLQIKQPFFGDKELETAYHKKYPTIYHLRKHLADADKNSPVADIREVYMAISHILKYRGHFLTLDKINPNNINMQNSWIDFIESCQEVFDLEISDESKNIADIFKSSENRQEKVKKILPYFQQELLKKDKSIFKQLLQLLFGLKTKFKDCFELEEEPDLNFSKENYDENLENFLGSLEEDFSDVFAKLKVLRDTILLSGMLTYTGATHARFSATMVERYEEHRKDLQRFKFFIKQNLSEQDYLDIFGRKTQNGFDVDKETKGYVGYITNKMVLTNPQKQKTIQQNFYDYISGKITGIEGAEYFLNKISDGTFLRKLRTSDNGAIPNQIHAYELEKIIERQGKDYPFLLENKDKLLSILTFKIPYYVGPLAKGSNSRFAWIKRATSSDILDDNDEDTRNGKIRPWNYQKLINMDETRDAFITNLIGNDIILLNEKVLPKRSLIYEEVMLQNELTRVKYKDKYGKAHFFDSELRQNIINGLFKNNSKRVNAKSLIKYLSDNHKDLNAIEIVSGVEKGKSFNSTLKTYNDLKTIFSEELLDSEIYQKELEEIIKVITVFDDKKSIKNYLTKFFGHLEILDEEKINQLSKLRYSGWGRYSAKLLLDIRDEDTGFNLLQFLRNDEENRNLTKLISDNTLSFEPKIKDIQSKSTIEDDIFDEIKKLAGSPAIKRGILNSIKIVDELVQIIGYPPHNIVIEMARENMTTEEGQKKAKTRKTKLESALKNIENSLLENGKVPHSDEQLQSEKLYLYYLQNGKDMYTLDKTGSPAPLYLDQLDQYEVDHIIPYSFLPIDSIDNKVLTHRENNQQKLNNIPDKETVANMKPFWEKLYNAKLISQTKYQRLTTSERTPDGVLTESMKAGFIERQLVETRQIIKHVARILDNRFSDTKIITLKSQLITNFRNTFHIAKIRELNDYHHAHDAYLAVVVGQTLLKVYPKLAPELIYGHHAHFNRHEENKATLRKHLYSNIMRFFNNPDSKVSKDIWDCNRDLPIIKDVIYNSQINFVKRTMIKKGAFYNQNPVGKFNKQLAANNRYPLKTKALCLDTSIYGGYGPMNSALSIIIIAERFNEKKGKIETVKEFHDIFIIDYEKFNNNPFQFLNDTSENGFLKKNNINRVLGFYRIPKYSLMQKIDGTRMLFESKSNLHKATQFKLTKTQNELFFHMKRLLTKSNLMDLKSKSAIKESQNFILKHKEEFDNISNQLSAFSQKMLGNTTSLKNLIKGYNERKIKEIDIRDETIKYFYDNFIKMFSFVKSGAPKDINDFFDNKCTVARMRPKPDKKLLNATLIHQSITGLYETRIDLSKLGED

SEQ ID NO: 62 - protein, 1181 aa, Actinomyces sp.
MLHCIAVIRVPPSEEPGFFETHADSCALCHHGCMTYAANDKAIRYRVGIDVGLRSIGFCAVEVDDEDHPIRILNSVVHVHDAGTGGPGETESLRKRSGVAARARRRGRAEKQRLKKLDVLLEELGWGVSSNELLDSHAPWHIRKRLVSEYIEDETERRQCLSVAMAHIARHRGWRNSFSKVDTLLLEQAPSDRMQGLKERVEDRTGLQFSEEVTQGELVATLLEHDGDVTIRGFVRKGGKATKVHGVLEGKYMQSDLVAELRQICRTQRVSETTFEKLVLSIFHSKEPAPSAARQRERVGLDELQLALDPAAKQPRAERAHPAFQKFKVVATLANMRIREQSAGERSLTSEELNRVARYLLNHTESESPTWDDVARKLEVPRHRLRGSSRASLETGGGLTYPPVDDTTVRVMSAEVDWLADWWDCANDESRGHMIDAISNGCGSEPDDVEDEEVNELISSATAEDMLKLELLAKKLPSGRVAYSLKTLREVTAAILETGDDLSQAITRLYGVDPGWVPTPAPIEAPVGNPSVDRVLKQVARWLKFASKRWGVPQTVNIEHTREGLKSASLLEEERERWERFEARREIRQKEMYKRLGISGPFRRSDQVRYEILDLQDCACLYCGNEINFQTFEVDHIIPRVDASSDSRRTNLAAVCHSCNSAKGGLAFGQWVKRGDCPSGVSLENAIKRVRSWSKDRLGLTEKAMGKRKSEVISRLKTEMPYEEFDGRSMESVAWMAIELKKRIEGYFNSDRPEGCAAVQVNAYSGRLTACARRAAHVDKRVRLIRLKGDDGHHKNRFDRRNHAMDALVIALMTPAIARTIAVREDRREAQQLTRAFESWKNFLGSEERMQDRWESWIGDVEYACDRLNELIDADKIPVTENLRLRNSGKLHADQPESLKKARRGSKRPRPQRYVLGDALPADVINRVTDPGLWTALVRAPGFDSQLGLPADLNRGLKLRGKRISADFPIDYFPTDSPALAVQGGYVGLEFHHARLYRIIGPKEKVKYALLRVCAIDLCGIDCDDLFEVELKPSSISMRTADAKLKEAMGNGSAKQIGWLVLGDEIQIDPTKFPKQSIGKFLKECGPVSSWRVSALDTPSKITLKPRLLSNEPLLKTSRVGGHESDLVVAECVEKIMKKTGWVVEINALCQSGLIRVIRRNALGEVRTSPKSGLPISLNLR

SEQ ID NO: 63 - protein, 1059 aa, Rhodovulum sp.
MGIRFAFDLGTNSIGWAVWRTGPGVFGEDTAASLDGSGVLIFKDGRNPKDGQSLATMRRVPRQSRKRRDRFVLRRRDLLAALRKAGLFPVDVEEGRRLAATDPYHLRAKALDESLTPHEMGRVIFHLNQRRGFRSNRKADRQDREKGKIAEGSKRLAETLAATNCRTLGEFLWSRHRGTPRTRSPTRIRMEGEGAKALYAFYPTREMVRAEFERLWTAQSRFAPDLLTPERHEEIAGILFRQRDLAPPKIGCCTFEPSERRLPRALPSVEARGIYERLAHLRITTGPVSDRGLTRPERDVLASALLAGKSLTFKAVRKTLKILPHALVNFEEAGEKGLDGALTAKLLSKPDHYGAAWHGLSFAEKDTFVGKLLDEADEERLIRRLVTENRLSEDAARRCASIPLADGYGRLGRTANTEILAALVEETDETGTVVTYAEAVRRAGERTGRNWHHSDERDGVILDRLPYYGEILQRHVVPGSGEPEEKNEAARWGRLANPTVHIGLNQLRKVVNRLIAAHGRPDQIVVELARELKLNREQKERLDRENRKNREENERRTAILAEHGQRDTAENKIRLRLFEEQARANAGIALCPYTGRAIGIAELFTSEVEIDHILPVSLTLDDSLANRVLCRREANREKRRQTPFQAFGATPAWNDIVARAAKLPPNKRWRFDPAALERFEREGGFLGRQLNETKYLSRLAKIYLGKICDPDRVYVTPGTLTGLLRARWGLNSILSDSNFKNRSDHRHHAVDAVVIGVLTRGMIQRIAHDAARAEDQDLDRVFRDVPVPFEDFRDHVRERVSTITVAVKPEHGKGGALHEDTSYGLVPDTDPNAALGNLVVRKPIRSLTAGEVDRVRDRALRARLGALAAPFRDESGRVRDAKGLAQALEAFGAENGIRRVRILKPDASVVTIADRRTGVPYRAVAPGENHHVDIVQMRDGSWRGFAASVFEVNRPGWRPEWEVKKLGGKLVMRLHKGDMVELSDKDGQRRVKVVQQIEISANRVRLSPHNDGGKLQDRHADADDPFRWDLATIPLLKDRGCVAVRVDPIGVVTLRRSNV

SEQ ID NO: 64 - protein, 1420 aa, Bifidobacterium bifidum
MSRKNYVDDYAISLDIGNASVGWSAFTPNYRLVRAKGHELIGVRLFDPADTAESRRMARTTRRRYSRRRWRLRLLDALFDQALSEIDPSFLARRKYSWVHPDDENNADCWYGSVLFDSNEQDKRFYEKYPTIYHLRKALMEDDSQHDIREIYLAIHHMVKYRGNFLVEGTLESSNAFKEDELLKLLGRITRYEMSEGEQNSDIEQDDENKLVAPANGQLADALCATRGSRSMRVDNALEALSAVNDLSREQRAIVKAIFAGLEGNKLDLAKIFVSKEFSSENKKILGIYFNKSDYEEKCVQIVDSGLLDDEEREFLDRMQGQYNAIALKQLLGRSTSVSDSKCASYDAHRANWNLIKLQLRTKENEKDINENYGILVGWKIDSGQRKSVRGESAYENMRKKANVFFKKMIETSDLSETDKNRLIHDIEEDKLFPIQRDSDNGVIPHQLHQNELKQIIKKQGKYYPFLLDAFEKDGKQINKIEGLLTFRVPYFVGPLVVPEDLQKSDNSENHWMVRKKKGEITPWNFDEMVDKDASGRKFIERLVGTDSYLLGEPTLPKNSLLYQEYEVLNELNNVRLSVRTGNHWNDKRRMRLGREEKTLLCQRLFMKGQTVTKRTAENLLRKEYGRTYELSGLSDESKFTSSLSTYGKMCRIFGEKYVNEHRDLMEKIVELQTVFEDKETLLHQLRQLEGISEADCALLVNTHYTGWGRLSRKLLTTKAGECKISDDFAPRKHSIIEIMRAEDRNLMEIITDKQLGFSDWIEQENLGAENGSSLMEVVDDLRVSPKVKRGIIQSIRLIDDISKAVGKRPSRIFLELADDIQPSGRTISRKSRLQDLYRNANLGKEFKGIADELNACSDKDLQDDRLFLYYTQLGKDMYTGEELDLDRLSSAYDIDHIIPQAVTQNDSIDNRVLVARAENARKTDSFTYMPQIADRMRNFWQILLDNGLISRVKFERLTRQNEFSEREKERFVQRSLVETRQIMKNVATLMRQRYGNSAAVIGLNAELTKEMHRYLGFSHKNRDINDYHHAQDALCVGIAGQFAANRGFFADGEVSDGAQNSYNQYLRDYLRGYREKLSAEDRKQGRAFGFIVGSMRSQDEQKRVNPRTGEVVWSEEDKDYLRKVMNYRKMLVTQKVGDDFGALYDETRYAATDPKGIKGIPFDGAKQDTSLYGGFSSAKPAYAVLIESKGKTRLVNVTMQEYSLLGDRPSDDELRKVLAKKKSEYAKANILLRHVPKMQLIRYGGGLMVIKSAGELNNAQQLWLPYEEYCYFDDLSQGKGSLEKDDLKKLLDSILGSVQCLYPWHRFTEEELADLHVAFDKLPEDEKKNVITGIVSALHADAKTANLSIVGMTGSWRRMNNKSGYTFSDEDEFIFQSPSGLFEKRVTVGELKRKAKKEVNSKYRTNEKRLPTLSGASQP

SEQ ID NO: 65 - protein, 1153 aa, Barnesiella intestinihominis
MKNILGLDLGLSSIGWSVIRENSEEQELVAMGSRVVSLTAAELSSFTQGNGVSINSQRTQKRTQRKGYDRYQLRRTLLRNKLDTLGMLPDDSLSYLPKLQLWGLRAKAVTQRIELNELGRVLLHLNQKRGYKSIKSDFSGDKKITDYVKTVKTRYDELKEMRLTIGELFFRRLTENAFFRCKEQVYPRQAYVEEFDCIMNCQRKFYPDILTDETIRCIRDEIIYYQRPLKSCKYLVSRCEFEKRFYLNAAGKKTEAGPKVSPRTSPLFQVCRLWESINNIVVKDRRNEIVFISAEQRAALFDFLNTHEKLKGSDLLKLLGLSKTYGYRLGEQFKTGIQGNKTRVEIERALGNYPDKKRLLQFNLQEESSSMVNTETGEIIPMISLSFEQEPLYRLWHVLYSIDDREQLQSVLRQKFGIDDDEVLERLSAIDLVKAGFGNKSSKAIRRILPFLQLGMNYAEACEAAGYNHSNNYTKAENEARALLDRLPAIKKNELRQPVVEKILNQMVNVVNALMEKYGRFDEIRVELARELKQSKEERSNTYKSINKNQRENEQIAKRIVEYGVPTRSRIQKYKMWEESKHCCIYCGQPVDVGDFLRGFDVEVEHIIPKSLYFDDSFANKVCSCRSCNKEKNNRTAYDYMKSKGEKALSDYVERVNTMYTNNQISKTKWQNLLTPVDKISIDFIDRQLRESQYIARKAKEILTSICYNVTATSGSVTSFLRHVWGWDTVLHDLNFDRYKKVGLTEVIEVNHRGSVIRREQIKDWSKRFDHRHHAIDALTIACTKQAYIQRLNNLRAEEGPDFNKMSLERYIQSQPHFSVAQVREAVDRILVSFRAGKRAVTPGKRYIRKNRKRISVQSVLIPRGALSEESVYGVIHVWEKDEQGHVIQKQRAVMKYPITSINREMLDKEKVVDKRIHRILSGRLAQYNDNPKEAFAKPVYIDKECRIPIRTVRCFAKPAINTLVPLKKDDKGNPVAWVNPGNNHHVAIYRDEDGKYKERTVTFWEAVDRCRVGIPAIVTQPDTIWDNILQRNDISENVLESLPDVKWQFVLSLQQNEMFILGMNEEDYRYAMDQQDYALLNKYLYRVQKLSKSDYSFRYHTETSVEDKYDGKPNLKLSMQMGKLKRVSIKSLLGLNPHKVHISVLGEIKEIS

SEQ ID NO: 66 - protein, 1052 aa, Aminomonas paucivorans
MIGEHVRGGCLFDDHWTPNWGAFRLPNTVRTFTKAENPKDGSSLAEPRRQARGLRRRLRRKTQRLEDLRRLLAKEGVLSLSDLETLFRETPAKDPYQLRAEGLDRPLSFPEWVRVLYHITKHRGFQSNRRNPVEDGQERSRQEEEGKLLSGVGENERLLREGGYRTAGEMLARDPKFQDHRRNRAGDYSHTLSRSLLLEEARRLFQSQRTLGNPHASSNLEEAFLHLVAFQNPFASGEDIRNKAGHCSLEPDQIRAPRRSASAETFMLLQKTGNLRLIHRRTGEERPLTDKEREQIHLLAWKQEKVTHKTLRRHLEIPEEWLFTGLPYHRSGDKAEEKLFVHLAGIHEIRKALDKGPDPAVWDTLRSRRDLLDSIADTLTFYKNEDEILPRLESLGLSPENARALAPLSFSGTAHLSLSALGKLLPHLEEGKSYTQARADAGYAAPPPDRHPKLPPLEEADWRNPVVFRALTQTRKVVNALVRRYGPPWCIHLETARELSQPAKVRRRIETEQQANEKKKQQAEREFLDIVGTAPGPGDLLKMRLWREQGGFCPYCEEYLNPTRLAEPGYAEMDHILPYSRSLDNGWHNRVLVHGKDNRDKGNRTPFEAFGGDTARWDRLVAWVQASHLSAPKKRNLLREDFGEEAERELKDRNLTDTRFITKTAATLLRDRLTFHPEAPKDPVMTLNGRLTAFLRKQWGLHKNRKNGDLHHALDAAVLAVASRSFVYRLSSHNAAWGELPRGREAENGFSLPYPAFRSEVLARLCPTREEILLRLDQGGVGYDEAFRNGLRPVFVSRAPSRRLRGKAHMETLRSPKWKDHPEGPRTASRIPLKDLNLEKLERMVGKDRDRKLYEALRERLAAFGGNGKKAFVAPFRKPCRSGEGPLVRSLRIFDSGYSGVELRDGGEVYAVADHESMVRVDVYAKKNRFYLVPVYVADVARGIVKNRAIVAHKSEEEWDLVDGSFDFRFSLFPGDLVEIEKKDGAYLGYYKSCHRGDGRLLLDRHDRMPRESDCGTFYVSTRKDVLSMSKYQVDPLGEIRLVGSEKPPFVL

SEQ ID NO: 67 - protein, 1062 aa, Ralstonia syzygii
MAEKQHRWGLDIGTNSIGWAVIALIEGRPAGLVATGSRIFSDGRNPKDGSSLAVERRGPRQMRRRRDRYLRRRDRFMQALINVGLMPGDAAARKALVTENPYVLRQRGLDQALTLPEFGRALFHLNQRRGFQSNRKTDRATAKESGKVKNAIAAFRAGMGNARTVGEALARRLEDGRPVRARMVGQGKDEHYELYIAREWIAQEFDALWASQQRFHAEVLADAARDRLRAILLFQRKLLPVPVGKCFLEPNQPRVAAALPSAQRFRLMQELNHLRVMTLADKRERPLSFQERNDLLAQLVARPKCGFDMLRKIVFGANKEAYRFTIESERRKELKGCDTAAKLAKVNALGTRWQALSLDEQDRLVCLLLDGENDAVLADALREHYGLTDAQIDTLLGLSFEDGHMRLGRSALLRVLDALESGRDEQGLPLSYDKAVVAAGYPAHTADLENGERDALPYYGELLWRYTQDAPTAKNDAERKFGKIANPTVHIGLNQLRKLVNALIQRYGKPAQIVVELARNLKAGLEEKERIKKQQTANLERNERIRQKLQDAGVPDNRENRLRMRLFEELGQGNGLGTPCIYSGRQISLQRLFSNDVQVDHILPFSKTLDDSFANKVLAQHDANRYKGNRGPFEAFGANRDGYAWDDIRARAAVLPRNKRNRFAETAMQDWLHNETDFLARQLTDTAYLSRVARQYLTAICSKDDVYVSPGRLTAMLRAKWGLNRVLDGVMEEQGRPAVKNRDDHRHHAIDAVVIGATDRAMLQQVATLAARAREQDAERLIGDMPTPWPNFLEDVRAAVARCVVSHKPDHGPEGGLHNDTAYGIVAGPFEDGRYRVRHRVSLFDLKPGDLSNVRCDAPLQAELEPIFEQDDARAREVALTALAERYRQRKVWLEELMSVLPIRPRGEDGKTLPDSAPYKAYKGDSNYCYELFINERGRWDGELISTFRANQAAYRRFRNDPARFRRYTAGGRPLLMRLCINDYIAVGTAAERTIFRVVKMSENKITLAEHFEGGTLKQRDADKDDPFKYLTKSPGALRDLGARRIFVDLIGRVLDPGIKGD

SEQ ID NO: 68 - protein, 1329 aa, Catenibacterium mitsuokai
IVDYCIGLDLGTGSVGWAVVDMNHRLMKRNGKHLWGSRLFSNAETAANRRASRSIRRRYNKRRERIRLLRAILQDMVLEKDPTFFIRLEHTSFLDEEDKAKYLGTDYKDNYNLFIDEDFNDYTYYHKYPTIYHLRKALCESTEKADPRLIYLALHHIVKYRGNFLYEGQKFNMDASNIEDKLSDIFTQFTSFNNIPYEDDEKKNLEILEILKKPLSKKAKVDEVMTLIAPEKDYKSAFKELVTGIAGNKMNVTKMILCEPIKQGDSEIKLKFSDSNYDDQFSEVEKDLGEYVEFVDALHNVYSWVELQTIMGATHTDNASISEAMVSRYNKHHDDLKLLKDCIKNNVPNKYFDMFRNDSEKSKGYYNYINRPSKAPVDEFYKYVKKCIEKVDTPEAKQILNDIELENFLLKQNSRTNGSVPYQMQLDEMIKIIDNQAEYYPILKEKREQLLSILTFRIPYYFGPLNETSEHAWIKRLEGKENQRILPWNYQDIVDVDATAEGFIKRMRSYCTYFPDEEVLPKNSLIVSKYEVYNELNKIRVDDKLLEVDVKNDIYNELFMKNKTVTEKKLKNWLVNNQCCSKDAEIKGFQKENQFSTSLTPWIDFTNIFGKIDQSNFDLIENIIYDLTVFEDKKIMKRRLKKKYALPDDKVKQILKLKYKDWSRLSKKLLDGIVADNRFGSSVTVLDVLEMSRLNLMEIINDKDLGYAQMIEEATSCPEDGKFTYEEVERLAGSPALKRGIWQSLQIVEEITKVMKCRPKYIYIEFERSEEAKERTESKIKKLENVYKDLDEQTKKEYKSVLEELKGFDNTKKISSDSLFLYFTQLGKCMYSGKKLDIDSLDKYQIDHIVPQSLVKDDSFDNRVLVVPSENQRKLDDLVVPFDIRDKMYRFWKLLFDHELISPKKFYSLIKTEYTERDEERFINRQLVETRQITKNVTQIIEDHYSTTKVAAIRANLSHEFRVKNHIYKNRDINDYHHAHDAYIVALIGGFMRDRYPNMHDSKAVYSEYMKMFRKNKNDQKRWKDGFVINSMNYPYEVDGKLIWNPDLINEIKKCFYYKDCYCTTKLDQKSGQLFNLTVLSNDAHADKGVTKAVVPVNKNRSDVHKYGGFSGLQYTIVAIEGQKKKGKKTELVKKISGVPLHLKAASINEKINYIEEKEGLSDVRIIKDNIPVNQMIEMDGGEYLLTSPTEYVNARQLVLNEKQCALIADIYNAIYKQDYDNLDDILMIQLYIELTNKMKVLYPAYRGIAEKFESMNENYVVISKEEKANIIKQMLIVMHRGPQNGNIVYDDFKISDRIGRLKTKNHNLNNIVFISQSPTGIYTKKYKL

SEQ ID NO: 69 - protein, 1314 aa, Mycoplasma synoviae
MLRLYCANNLVLNNVQNLWKYLLLLIFDKKIIFLFKIKVILIRRYMENNNKEKIVIGFDLGVASVGWSIVNAETKEVIDLGVRLFSEPEKADYRRAKRTTRRLLRRKKFKREKFHKLILKNAEIFGLQSRNEILNVYKDQSSKYRNILKLKINALKEEIKPSELVWILRDYLQNRGYFYKNEKLTDEFVSNSFPSKKLHEHYEKYGFFRGSVKLDNKLDNKKDKAKEKDEEEESDAKKESEELIFSNKQWINEIVKVFENQSYLTESFKEEYLKLFNYVRPFNKGPGSKNSRTAYGVFSTDIDPETNKFKDYSNIWDKTIGKCSLFEEEIRAPKNLPSALIFNLQNEICTIKNEFTEFKNWWLNAEQKSEILKFVFTELFNWKDKKYSDKKFNKNLQDKIKKYLLNFALENFNLNEEILKNRDLENDTVLGLKGVKYYEKSNATADAALEFSSLKPLYVFIKFLKEKKLDLNYLLGLENTEILYFLDSIYLAISYSSDLKERNEWFKKLLKELYPKIKNNNLEIIENVEDIFEITDQEKFESFSKTHSLSREAFNHIIPLLLSNNEGKNYESLKHSNEELKKRTEKAELKAQQNQKYLKDNFLKEALVPLSVKTSVLQAIKIFNQIIKNFGKKYEISQVVIEMARELTKPNLEKLLNNATNSNIKILKEKLDQTEKFDDFTKKKFIDKIENSVVFRNKLFLWFEQDRKDPYTQLDIKINEIEDETEIDHVIPYSKSADDSWFNKLLVKKSTNQLKKNKTVWEYYQNESDPEAKWNKFVAWAKRIYLVQKSDKESKDNSEKNSIFKNKKPNLKFKNITKKLFDPYKDLGFLARNLNDTRYATKVFRDQLNNYSKHHSKDDENKLFKVVCMNGSITSFLRKSMWRKNEEQVYRFNFWKKDRDQFFHHAVDASIIAIFSLLTKTLYNKLRVYESYDVQRREDGVYLINKETGEVKKADKDYWKDQHNFLKIRENAIEIKNVLNNVDFQNQVRYSRKANTKLNTQLFNETLYGVKEFENNFYKLEKVNLFSRKDLRKFILEDLNEESEKNKKNENGSRKRILTEKYIVDEILQILENEEFKDSKSDINALNKYMDSLPSKFSEFFSQDFINKCKKENSLILTFDAIKHNDPKKVIKIKNLKFFREDATLKNKQAVHKDSKNQIKSFYESYKCVGFIWLKNKNDLEESIFVPINSRVIHFGDKDKDIFDFDSYNKEKLLNEINLKRPENKKFNSINEIEFVKFVKPGALLLNFENQQIYYISTLESSSLRAKIKLLNKMDKGKAVSMKKITNPDEYKIIEHVNPLGINLNWTKKLENNN

SEQ ID NO: 70 - protein, 1473 aa, Flavobacterium branchiophilum
MAKILGLDLGTNSIGWAVVERENIDFSLIDKGVRIFSEGVKSEKGIESSRAAERTGYRSARKIKYRRKLRKYETLKVLSLNRMCPLSIEEVEEWKKSGFKDYPLNPEFLKWLSTDEESNVNPYFFRDRASKHKVSLFELGRAFYHIAQRRGFLSNRLDQSAEGILEEHCPKIEAIVEDLISIDEISTNITDYFFETGILDSNEKNGYAKDLDEGDKKLVSLYKSLLAILKKNESDFENCKSEIIERLNKKDVLGKVKGKIKDISQAMLDGNYKTLGQYFYSLYSKEKIRNQYTSREEHYLSEFITICKVQGIDQINEEEKINEKKFDGLAKDLYKAIFFQRPLKSQKGLIGKCSFEKSKSRCAISHPDFEEYRMWTYLNTIKIGTQSDKKLRFLTQDEKLKLVPKFYRKNDFNFDVLAKELIEKGSSFGFYKSSKKNDFFYWFNYKPTDTVAACQVAASLKNAIGEDWKTKSFKYQTINSNKEQVSRTVDYKDLWHLLTVATSDVYLYEFAIDKLGLDEKNAKAFSKTKLKKDFASLSLSAINKILPYLKEGLLYSHAVFVANIENIVDENIWKDEKQRDYIKTQISEIIENYTLEKSRFEIINGLLKEYKSENEDGKRVYYSKEAEQSFENDLKKKLVLFYKSNEIENKEQQETIFNELLPIFIQQLKDYEFIKIQRLDQKVLIFLKGKNETGQIFCTEEKGTAEEKEKKIKNRLKKLYHPSDIEKFKKKIIKDEFGNEKIVLGSPLTPSIKNPMAMRALHQLRKVLNALILEGQIDEKTIIHIEMARELNDANKRKGIQDYQNDNKKFREDAIKEIKKLYFEDCKKEVEPTEDDILRYQLWMEQNRSEIYEEGKNISICDIIGSNPAYDIEHTIPRSRSQDNSQMNKTLCSQRFNREVKKQSMPIELNNHLEILPRIAHWKEEADNLTREIEIISRSIKAAATKEIKDKKIRRRHYLTLKRDYLQGKYDRFIWEEPKVGFKNSQIPDTGIITKYAQAYLKSYFKKVESVKGGMVAEFRKIWGIQESFIDENGMKHYKVKDRSKHTHHTIDAITIACMTKEKYDVLAHAWTLEDQQNKKEARSIIEASKPWKTFKEDLLKIEEEILVSHYTPDNVKKQAKKIVRVRGKKQFVAEVERDVNGKAVPKKAASGKTIYKLDGEGKKLPRLQQGDTIRGSLHQDSIYGAIKNPLNTDEIKYVIRKDLESIKGSDVESIVDEVVKEKIKEAIANKVLLLSSNAQQKNKLVGTVWMNEEKRIAINKVRIYANSVKNPLHIKEHSLLSKSKHVHKQKVYGQNDENYAMAIYELDGKRDFELINIFNLAKLIKQGQGFYPLHKKKEIKGKIVFVPIEKRNKRDVVLKRGQQVVFYDKEVENPKDISEIVDFKGRIYIIEGLSIQRIVRPSGKVDEYGVIMLRYFKEARKADDIKQDNFKPDGVFKLGENKPTRKMNHNQFTAFVEGIDFKVLPSGKFEKI

SEQ ID NO: 71 - protein, 1391 aa, Eubacterium yurii
MENKQYYIGLDVGTNSVGWAVTDTSYNLLRAKGKDMWGARLFEKANTAAERRTKRTSRRRSEREKARKAMLKELFADEINRVDPSFFIRLEESKFFLDDRSENNRQRYTLFNDATFTDKDYYEKYKTIFHLRSALINSDEKFDVRLVFLAILNLFSHRGHFLNASLKGDGDIQGMDVFYNDLVESCEYFEIELPRITNIDNFEKILSQKGKSRTKILEELSEELSISKKDKSKYNLIKLISGLEASVVELYNIEDIQDENKKIKIGFRESDYEESSLKVKEIIGDEYFDLVERAKSVHDMGLLSNIIGNSKYLCEARVEAYENHHKDLLKIKELLKKYDKKAYNDMFRKMTDKNYSAYVGSVNSNIAKERRSVDKRKIEDLYKYIEDTALKNIPDDNKDKIEILEKIKLGEFLKKQLTASNGVIPNQLQSRELRAILKKAENYLPFLKEKGEKNLTVSEMIIQLFEFQIPYYVGPLDKNPKKDNKANSWAKIKQGGRILPWNFEDKVDVKGSRKEFIEKMVRKCTYISDEHTLPKQSLLYEKFMVLNEINNIKIDGEKISVEAKQKIYNDLFVKGKKVSQKDIKKELISLNIMDKDSVLSGTDTVCNAYLSSIGKFTGVFKEEINKQSIVDMIEDIIFLKTVYGDEKRFVKEEIVEKYGDEIDKDKIKRILGFKFSNWGNLSKSFLELEGADVGTGEVRSIIQSLWETNFNLMELLSSRFTYMDELEKRVKKLEKPLSEWTIEDLDDMYLSSPVKRMIWQSMKIVDEIQTVIGYAPKRIFVEMTRSEGEKVRTKSRKDRLKELYNGIKEDSKQWVKELDSKDESYFRSKKMYLYYLQKGRCMYSGEVIELDKLMDDNLYDIDHIYPRSFVKDDSLDNLVLVKKEINNRKQNDPITPQIQASCQGFWKILHDQGFMSNEKYSRLTRKTQEFSDEEKLSFINRQIVETGQATKCMAQILQKSMGEDVDVVFSKARLVSEFRHKFELFKSRLINDFHHANDAYLNIVVGNSYFVKFTRNPANFIKDARKNPDNPVYKYHMDRFFERDVKSKSEVAWIGQSEGNSGTIVIVKKTMAKNSPLITKKVEEGHGSITKETIVGVKEIKFGRNKVEKADKTPKKPNLQAYRPIKTSDERLCNILRYGGRTSISISGYCLVEYVKKRKTIRSLEAIPVYLGRKDSLSEEKLLNYFRYNLNDGGKDSVSDIRLCLPFISTNSLVKIDGYLYYLGGKNDDRIQLYNAYQLKMKKEEVEYIRKIEKAVSMSKFDEIDREKNPVLTEEKNIELYNKIQDKFENTVFSKRMSLVKYNKKDLSFGDFLKNKKSKFEEIDLEKQCKVLYNIIFNLSNLKEVDLSDIGGSKSTGKCRCKKNITNYKEFKLIQQSITGLYSCEKDLMTI

SEQ ID NO: 72 - protein, 1131 aa, unidentified
MAQHVFGLDIGIASVGWAILGEQRIIDLGVRCFDKAETAKEGDPLNLTRRQARLLRRRLYRRAWRLTQLSRLLKRKGLIADAKLFAKAPSYGDSAWELRRQGLDRLLTPLEWARVIYHQCKHRGFHWTSKAEEAKADSDAEGGRVKQGLAHTKALMQAKNYRSAAEMVLAEFPDAQRNKRGQYDKALSRVLLGEELALLFATQRRLGNPHASDFFEKLILGDGDRKSGLFWQQKPALSGADLLKMLGKCTFEKGEYRAPKASFSVERHVWLTRLNNLRIVVDGRSRPLNEAERQAALLLPYQTETSKYKTLKNAFIKAGLWGDGVRFGGLAYPSQAQIDAEKTKDPEDQFLVKLPAWHELRKAFKAAGHEALWQQISTPALDGDPTLLDQIATVLSVYKDGAEVVQQLRQLALPEPAASIAVLEKISFDKFSSLSLKALRRIVPLMQSGLRYDEAVAQIPEYGHHSQRIEPGAAKHLYLPPFYEAQRKYAGKGDHIGSMQFRDDADIPRNPVVLRALNQARKVVNALIREYGSPIAVNIEMARDLSRPLDERNKVKRAQEEFRDRNDRARSEFERDFGYKPKAAAFEKWMLYREQLGQCAYSQQPLDIQRVLDDHNYAQVDHALPYSRSYDDSKNNKVLVLTHENQNKGNRTAFEYLTSFPDGEDGERWRTFVAWVQGNKAYRMAKRNRLLRKNYGVDESKGFIDRNLNDTRYICKFFKNYVEEHLQLAARADGDTARRCVVVNGQLTAFLRARWGLTKVRGDSDRHHALDAAVVAACTHGMVKALADYSRRKEISFLQEGFPDPETGEILNPAAFDRARQHFPEPWTHFAHELKARLFTDDLAALREDMQRLGSYTTEDLGRLRTLFVSRAPQRRSGGAVHKETIYAQPESLKQQGGVIEKILLTSLKLQDFDKLLNPESNDHFVEPHRNERLYAAIRQRLEQFGGRADKAFGPDNLFHKPDKNNQPTGPVVRSIKLVRGKQTGIPIRGGLAKNDSMLRVDIFTKAGKFHLVPVYVHHRVTGLPNRAIVAFKDEDEWTLIDESFAFLFSVYPNDYVKVTLKKEQQSGYYSGADRSTGAMNLWAHDRAASVGKDGLIRGIGVKTALSVEKFNVDVLGRIYLAPPETRSGLA

SEQ ID NO: 73 - protein, 1197 aa, Porphyromonas sp.
MLMSKHVLGLDLGVGSIGWCLIALDAQGDPAEILGMGSRVVPLNNATKAIEAFNAGAAFTASQERTARRTMRRGFARYQLRRYRLRRELEKVGMLPDAALIQLPLLELWELRERAATAGRRLTLPELGRVLCHINQKRGYRHVKSDAAAIVGDEGEKKKDSNSAYLAGIRANDEKLQAEHKTVGQYFAEQLRQNQSESPTGGISYRIKDQIFSRQCYIDEYDQIMAVQRVHYPDILTDEFIRMLRDEVIFMQRPLKSCKHLVSLCEFEKQERVMRVQQDDGKGGWQLVERRVKFGPKVAPKSSPLFQLCCIYEAVNNIRLTRPNGSPCDITPEERAKIVAHLQSSASLSFAALKKLLKEKALIADQLTSKSGLKGNSTRVALASALQPYPQYHHLLDMELETRMMTVQLTDEETGEVTEREVAVVTDSYVRKPLYRLWHILYSIEEREAMRRALITQLGMKEEDLDGGLLDQLYRLDFVKPGYGNKSAKFICKLLPQLQQGLGYSEACAAVGYRHSNSPTSEEITERTLLEKIPLLQRNELRQPLVEKILNQMINLVNALKAEYGIDEVRVELARELKMSREERERMARNNKDREERNKGVAAKIRECGLYPTKPRIQKYMLWKEAGRQCLYCGRSIEEEQCLREGGMEVEHIIPKSVLYDDSYGNKTCACRRCNKEKGNRTALEYIRAKGREAEYMKRINDLLKEKKISYSKHQRLRWLKEDIPSDFLERQLRLTQYISRQAMAILQQGIRRVSASEGGVTARLRSLWGYGKILHTLNLDRYDSMGETERVSREGEATEELHITNWSKRMDHRHHAIDALVVACTRQSYIQRLNRLSSEFGREDKKKEDQEAQEQQATETGRLSNLERWLTQRPHFSVRTVSDKVAEILISYRPGQRVVTRGRNIYRKKMADGREVSCVQRGVLVPRGELMEASFYGKILSQGRVRIVKRYPLHDLKGEVVDPHLRELITTYNQELKSREKGAPIPPLCLDKDKKQEVRSVRCYAKTLSLDKAIPMCFDEKGEPTAFVKSASNHHLALYRTPKGKLVESIVTFWDAVDRARYGIPLVITHPREVMEQVLQRGDIPEQVLSLLPPSDWVFVDSLQQDEMVVIGLSDEELQRALEAQNYRKISEHLYRVQKMSSSYYVFRYHLETSVADDKNTSGRIPKFHRVQSLKAYEERNIRKVRVDLLGRISLL

SEQ ID NO: 74 - protein, 1265 aa, Mycoplasma ovipneumoniae
MHNKKNITIGFDLGIASIGWAIIDSTTSKILDWGTRTFEERKTANERRAFRSTRRNIRRKAYRNQRFINLILKYKDLFELKNISDIQRANKKDTENYEKIISFFTEIYKKCAAKHSNILEVKVKALDSKIEKLDLIWILHDYLENRGFFYDLEEENVADKYEGIEHPSILLYDFFKKNGFFKSNSSIPKDLGGYSFSNLQWVNEIKKLFEVQEINPEFSEKFLNLFTSVRDYAKGPGSEHSASEYGIFQKDEKGKVFKKYDNIWDKTIGKCSFFVEENRSPVNYPSYEIFNLLNQLINLSTDLKTTNKKIWQLSSNDRNELLDELLKVKEKAKIISISLKKNEIKKIILKDFGFEKSDIDDQDTIEGRKIIKEEPTTKLEVTKHLLATIYSHSSDSNWININNILEFLPYLDAICIILDREKSRGQDEVLKKLTEKNIFEVLKIDREKQLDFVKSIFSNTKFNFKKIGNFSLKAIREFLPKMFEQNKNSEYLKWKDEEIRRKWEEQKSKLGKTDKKTKYLNPRIFQDEIISPGTKNTFEQAVLVLNQIIKKYSKENIIDAIIIESPREKNDKKTIEEIKKRNKKGKGKTLEKLFQILNLENKGYKLSDLETKPAKLLDRLRFYHQQDGIDLYTLDKINIDQLINGSQKYEIEHIIPYSMSYDNSQANKILTEKAENLKKGKLIASEYIKRNGDEFYNKYYEKAKELFINKYKKNKKLDSYVDLDEDSAKNRFRFLTLQDYDEFQVEFLARNLNDTRYSTKLFYHALVEHFENNEFFTYIDENSSKHKVKISTIKGHVTKYFRAKPVQKNNGPNENLNNNKPEKIEKNRENNEHHAVDAAIVAIIGNKNPQIANLLTLADNKTDKKFLLHDENYKENIETGELVKIPKFEVDKLAKVEDLKKIIQEKYEEAKKHTAIKFSRKTRTILNGGLSDETLYGFKYDEKEDKYFKIIKKKLVTSKNEELKKYFENPFGKKADGKSEYTVLMAQSHLSEFNKLKEIFEKYNGFSNKTGNAFVEYMNDLALKEPTLKAEIESAKSVEKLLYYNFKPSDQFTYHDNINNKSFKRFYKNIRIIEYKSIPIKFKILSKHDGGKSFKDTLFSLYSLVYKVYENGKESYKSIPVTSQMRNFGIDEFDFLDENLYNKEKLDIYKSDFAKPIPVNCKPVFVLKKGSILKKKSLDIDDFKETKETEEGNYYFISTISKRFNRDTAYGLKPLKLSVVKPVAEPSTNPIFKEYIPIHLDELGNEYPVKIKEHTDDEKLMCTIK

SEQ ID NO: 75 - protein, 1409 aa, Wolinella succinogenes
MLVSPISVDLGGKNTGFFSFTDSLDNSQSGTVIYDESFVLSQVGRRSKRHSKRNNLRNKLVKRLFLLILQEHHGLSIDVLPDEIRGLFNKRGYTYAGFELDEKKKDALESDTLKEFLSEKLQSIDRDSDVEDFLNQIASNAESFKDYKKGFEAVFASATHSPNKKLELKDELKSEYGENAKELLAGLRVTKEILDEFDKQENQGNLPRAKYFEELGEYIATNEKVKSFFDSNSLKLTDMTKLIGNISNYQLKELRRYFNDKEMEKGDIWIPNKLHKITERFVRSWHPKNDADRQRRAELMKDLKSKEIMELLTTTEPVMTIPPYDDMNNRGAVKCQTLRLNEEYLDKHLPNWRDIAKRLNHGKFNDDLADSTVKGYSEDSTLLHRLLDTSKEIDIYELRGKKPNELLVKTLGQSDANRLYGFAQNYYELIRQKVRAGIWVPVKNKDDSLNLEDNSNMLKRCNHNPPHKKNQIHNLVAGILGVKLDEAKFAEFEKELWSAKVGNKKLSAYCKNIEELRKTHGNTFKIDIEELRKKDPAELSKEEKAKLRLTDDVILNEWSQKIANFFDIDDKHRQRFNNLFSMAQLHTVIDTPRSGFSSTCKRCTAENRFRSETAFYNDETGEFHKKATATCQRLPADTQRPFSGKIERYIDKLGYELAKIKAKELEGMEAKEIKVPIILEQNAFEYEESLRKSKTGSNDRVINSKKDRDGKKLAKAKENAEDRLKDKDKRIKAFSSGICPYCGDTIGDDGEIDHILPRSHTLKIYGTVFNPEGNLIYVHQKCNQAKADSIYKLSDIKAGVSAQWIEEQVANIKGYKTFSVLSAEQQKAFRYALFLQNDNEAYKKVVDWLRTDQSARVNGTQKYLAKKIQEKLTKMLPNKHLSFEFILADATEVSELRRQYARQNPLLAKAEKQAPSSHAIDAVMAFVARYQKVFKDGTPPNADEVAKLAMLDSWNPASNEPLTKGLSTNQKIEKMIKSGDYGQKNMREVFGKSIFGENAIGERYKPIVVQEGGYYIGYPATVKKGYELKNCKVVTSKNDIAKLEKIIKNQDLISLKENQYIKIFSINKQTISELSNRYFNMNYKNLVERDKEIVGLLEFIVENCRYYTKKVDVKFAPKYIHETKYPFYDDWRRFDEAWRYLQENQNKTSSKDRFVIDKSSLNEYYQPDKNEYKLDVDTQPIWDDFCRWYFLDRYKTANDKKSIRIKARKTFSLLAESGVQGKVFRAKRKIPTGYAYQALPMDNNVIAGDYANILLEANSKTLSLVPKSGISIEKQLDKKLDVIKKTDVRGLAIDNNSFFNADFDTHGIRLIVENTSVKVGNFPISAIDKSAKRMIFRALFEKEKGKRKKKTTISFKESGPVQDYLKVFLKKIVKIQLRTDGSISNIVVRKNAADFTLSFRSEHIQKLLK

SEQ ID NO: 76 - protein, 1345 aa, Streptococcus mutans
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 77 - protein, 1218 aa, Prevotella timonensis
MNKRILGLDTGTNSLGWAVVDWDEHAQSYELIKYGDVIFQEGVKIEKGIESSKAAERSGYKAIRKQYFRRRLRKIQVLKVLVKYHLCPYLSDDDLRQWHLQKQYPKSDELMLWQRTSDEEGKNPYYDRHRCLHEKLDLTVEADRYTLGRALYHLTQRRGFLSNRLDTSADNKEDGVVKSGISQLSTEMEEAGCEYLGDYFYKLYDAQGNKVRIRQRYTDRNKHYQHEFDAICEKQELSSELIEDLQRAIFFQLPLKSQRHGVGRCTFERGKPRCADSHPDYEEFRMLCFVNNIQVKGPHDLELRPLTYEEREKIEPLFFRKSKPNFDFEDIAKALAGKKNYAWIHDKEERAYKFNYRMTQGVPGCPTIAQLKSIFGDDWKTGIAETYTLIQKKNGSKSLQEMVDDVWNVLYSFSSVEKLKEFAHHKLQLDEESAEKFAKIKLSHSFAALSLKAIRKFLPFLRKGMYYTHASFFANIPTIVGKEIWNKEQNRKYIMENVGELVFNYQPKHREVQGTIEMLIKDFLANNFELPAGATDKLYHPSMIETYPNAQRNEFGILQLGSPRTNAIRNPMAMRSLHILRRVVNQLLKESIIDENTEVHVEYARELNDANKRRAIADRQKEQDKQHKKYGDEIRKLYKEETGKDIEPTQTDVLKFQLWEEQNHHCLYTGEQIGITDFIGSNPKFDIEHTIPQSVGGDSTQMNLTLCDNRFNREVKKAKLPTELANHEEILTRIEPWKNKYEQLVKERDKQRTFAGMDKAVKDIRIQKRHKLQMEIDYWRGKYERFTMTEVPEGFSRRQGTGIGLISRYAGLYLKSLFHQADSRNKSNVYVVKGVATAEFRKMWGLQSEYEKKCRDNHSHHCMDAITIACIGKREYDLMAEYYRMEETFKQGRGSKPKFSKPWATFTEDVLNIYKNLLVVHDTPNNMPKHTKKYVQTSIGKVLAQGDTARGSLHLDTYYGAIERDGEIRYVVRRPLSSFTKPEELENIVDETVKRTIKEAIADKNFKQAIAEPIYMNEEKGILIKKVRCFAKSVKQPINIRQHRDLSKKEYKQQYHVMNENNYLLAIYEGLVKNKVVREFEIVSYIEAAKYYKRSQDRNIFSSIVPTHSTKYGLPLKTKLLMGQLVLMFEENPDEIQVDNTKDLVKRLYKVVGIEKDGRIKFKYHQEARKEGLPIFSTPYKNNDDYAPIFRQSINNINILVDGIDFTIDILGKVTLKE

SEQ ID NO: 78 - protein, 1021 aa, Clostridium cellulolyticum
MKYTLGLDVGIASVGWAVIDKDNNKIIDLGVRCFDKAEESKTGESLATARRIARGMRRRISRRSQRLRLVKKLFVQYEIIKDSSEFNRIFDTSRDGWKDPWELRYNALSRILKPYELVQVLTHITKRRGFKSNRKEDLSTTKEGVVITSIKNNSEMLRTKNYRTIGEMIFMETPENSNKRNKVDEYIHTIAREDLLNEIKYIFSIQRKLGSPFVTEKLEHDFLNIWEFQRPFASGDSILSKVGKCTLLKEELRAPTSCYTSEYFGLLQSINNLVLVEDNNTLTLNNDQRAKIIEYAHFKNEIKYSEIRKLLDIEPEILFKAHNLTHKNPSGNNESKKFYEMKSYHKLKSTLPTDIWGKLHSNKESLDNLFYCLTVYKNDNEIKDYLQANNLDYLIEYIAKLPTFNKFKHLSLVAMKRIIPFMEKGYKYSDACNMAELDFTGSSKLEKCNKLTVEPIIENVTNPVVIRALTQARKVINAIIQKYGLPYMVNIELAREAGMTRQDRDNLKKEHENNRKAREKISDLIRQNGRVASGLDILKWRLWEDQGGRCAYSGKPIPVCDLLNDSLTQIDHIYPYSRSMDDSYMNKVLVLTDENQNKRSYTPYEVWGSTEKWEDFEARIYSMHLPQSKEKRLLNRNFITKDLDSFISRNLNDTRYISRFLKNYIESYLQFSNDSPKSCVVCVNGQCTAQLRSRWGLNKNREESDLHHALDAAVIACADRKIIKEITNYYNERENHNYKVKYPLPWHSFRQDLMETLAGVFISRAPRRKITGPAHDETIRSPKHFNKGLTSVKIPLTTVTLEKLETMVKNTKGGISDKAVYNVLKNRLIEHNNKPLKAFAEKIYKPLKNGTNGAIIRSIRVETPSYTGVFRNEGKGISDNSLMVRVDVFKKKDKYYLVPIYVAHMIKKELPSKAIVPLKPESQWELIDSTHEFLFSLYQNDYLVIKTKKGITEGYYRSCHRGTGSLSLMPHFANNKNVKIDIGVRTAISIEKYNVDILGNKSIVKGEPRRGMEKYNSFKSN

SEQ ID NO: 79 - protein, 1629 aa, Francisella tularensis
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 80 - protein, 1168 aa, Azospirillum sp.
MARPAFRAPRREHVNGWTPDPHRISKPFFILVSWHLLSRVVIDSSSGCFPGTSRDHTDKFAEWECAVQPYRLSFDLGTNSIGWGLLNLDRQGKPREIRALGSRIFSDGRDPQDKASLAVARRLARQMRRRRDRYLTRRTRLMGALVRFGLMPADPAARKRLEVAVDPYLARERATRERLEPFEIGRALFHLNQRRGYKPVRTATKPDEEAGKVKEAVERLEAAIAAAGAPTLGAWFAWRKTRGETLRARLAGKGKEAAYPFYPARRMLEAEFDTLWAEQARHHPDLLTAEAREILRHRIFHQRPLKPPPVGRCTLYPDDGRAPRALPSAQRLRLFQELASLRVIHLDLSERPLTPAERDRIVAFVQGRPPKAGRKPGKVQKSVPFEKLRGLLELPPGTGFSLESDKRPELLGDETGARIAPAFGPGWTALPLEEQDALVELLLTEAEPERAIAALTARWALDEATAAKLAGATLPDFHGRYGRRAVAELLPVLERETRGDPDGRVRPIRLDEAVKLLRGGKDHSDFSREGALLDALPYYGAVLERHVAFGTGNPADPEEKRVGRVANPTVHIALNQLRHLVNAILARHGRPEEIVIELARDLKRSAEDRRREDKRQADNQKRNEERKRLILSLGERPTPRNLLKLRLWEEQGPVENRRCPYSGETISMRMLLSEQVDIDHILPFSVSLDDSAANKVVCLREANRIKRNRSPWEAFGHDSERWAGILARAEALPKNKRWRFAPDALEKLEGEGGLRARHLNDTRHLSRLAVEYLRCVCPKVRVSPGRLTALLRRRWGIDAILAEADGPPPEVPAETLDPSPAEKNRADHRHHALDAVVIGCIDRSMVQRVQLAAASAEREAAAREDNIRRVLEGFKEEPWDGFRAELERRARTIVVSHRPEHGIGGALHKETAYGPVDPPEEGFNLVVRKPIDGLSKDEINSVRDPRLRRALIDRLAIRRRDANDPATALAKAAEDLAAQPASRGIRRVRVLKKESNPIRVEHGGNPSGPRSGGPFHKLLLAGEVHHVDVALRADGRRWVGHWVTLFEAHGGRGADGAAAPPRLGDGERFLMRLHKGDCLKLEHKGRVRVMQVVKLEPSSNSVVVVEPHQVKTDRSKHVKISCDQLRARGARRVTVDPLGRVRVHAPGARVGIGGDAGRTAMEPAEDIS

SEQ ID NO: 81 - protein, 1364 aa, unidentified
MKNLKEYYIGLDIGTASVGWAVTDESYNIPKFNGKKMWGVRLFDDAKTAEERRTQRGSRRRLNRRKERINLLQDLFATEISKVDPNFFLRLDNSDLYREDKDEKLKSKYTLFNDKDFKDRDYHKKYPTIHHLIMDLIEDEGKKDIRLLYLACHYLLKNRGHFIFEGQKFDTKNSFDKSINDLKIHLRDEYNIDLEFNNEDLIEIITDTTLNKTNKKKELKNIVGDTKFLKAISAIMIGSSQKLVDLFEDGEFEETTVKSVDFSTTAFDDKYSEYEEALGDTISLLNILKSIYDSSILENLLKDADKSKDGNKYISKAFVKKFNKHGKDLKTLKRIIKKYLPSEYANIFRNKSINDNYVAYTKSNITSNKRTKASKFTKQEDFYKFIKKHLDTIKETKLNSSENEDLKLIDEMLTDIEFKTFIPKLKSSDNGVIPYQLKLMELKKILDNQSKYYDFLNESDEYGTVKDKVESIMEFRIPYYVGPLNPDSKYAWIKRENTKITPWNFKDIVDLDSSREEFIDRLIGRCTYLKEEKVLPKASLIYNEFMVLNELNNLKLNEFLITEEMKKAIFEELFKTKKKVTLKAVSNLLKKEFNLTGDILLSGTDGDFKQGLNSYIDFKNIIGDKVDRDDYRIKIEEIIKLIVLYEDDKTYLKKKIKSAYKNDFTDDEIKKIAALNYKDWGRLSKRFLTGIEGVDKTTGEKGSIIYFMREYNLNLMELMSGHYTFTEEVEKLNPVENRELCYEMVDELYLSPSVKRMLWQSLRVVDEIKRIIGKDPKKIFIEMARAKEAKNSRKESRKNKLLEFYKFGKKAFINEIGEERYNYLLNEINSEEESKFRWDNLYLYYTQLGRCMYSLEPIDLADLKSNNIYDQDHIYPKSKIYDDSLENRVLVKKNLNHEKGNQYPIPEKVLNKNAYGFWKILFDKGLIGQKKYTRLTRRTPFEERELAEFIERQIVETRQATKETANLLKNICQDSEIVYSKAENASRFRQEFDIIKCRTVNDLHHMHDAYLNIVVGNVYNTKFTKNPLNFIKDKDNVRSYNLENMFKYDVVRGSYTAWIADDSEGNVKAATIKKVKRELEGKNYRFTRMSYIGTGGLYDQNLMRKGKGQIPQKENTNKSNIEKYGGYNKASSAYFALIESDGKAGRERTLETIPIMVYNQEKYGNTEAVDKYLKDNLELQDPKILKDKIKINSLIKLDGFLYNIKGKTGDSLSIAGSVQLIVNKEEQKLIKKMDKFLVKKKDNKDIKVTSFDNIKEEELIKLYKTLSDKLNNGIYSNKRNNQAKNISEALDKFKEISIEEKIDVLNQIILLFQSYNNGCNLKSIGLSAKTGVVFIPKKLNYKECKLINQSITGLFENEVDLLNL

SEQ ID NO: 82 - protein, 1119 aa, Lactobacillus coryniformis
MGYRIGLDVGITSTGYAVLKTDKNGLPYKILTLDSVIYPRAENPQTGASLAEPRRIKRGLRRRTRRTKFRKQRTQQLFIHSGLLSKPEIEQILATPQAKYSVYELRVAGLDRRLTNSELFRVLYFFIGHRGFKSNRKAELNPENEADKKQMGQLLNSIEEIRKAIAEKGYRTVGELYLKDPKYNDHKRNKGYIDGYLSTPNRQMLVDEIKQILDKQRELGNEKLTDEFYATYLLGDENRAGIFQAQRDFDEGPGAGPYAGDQIKKMVGKDIFEPTEDRAAKATYTFQYFNLLQKMTSLNYQNTTGDTWHTLNGLDRQAIIDAVFAKAEKPTKTYKPTDFGELRKLLKLPDDARFNLVNYGSLQTQKEIETVEKKTRFVDFKAYHDLVKVLPEEMWQSRQLLDHIGTALTLYSSDKRRRRYFAEELNLPAELIEKLLPLNFSKFGHLSIKSMQNIIPYLEMGQVYSEATTNTGYDFRKKQISKDTIREEITNPVVRRAVTKTIKIVEQIIRRYGKPDGINIELARELGRNFKERGDIQKRQDKNRQTNDKIAAELTELGIPVNGQNIIRYKLHKEQNGVDPYTGDQIPFERAFSEGYEVDHIIPYSISWDDSYTNKVLTSAKCNREKGNRIPMVYLANNEQRLNALTNIADNIIRNSRKRQKLLKQKLSDEELKDWKQRNINDTRFITRVLYNYFRQAIEFNPELEKKQRVLPLNGEVTSKIRSRWGFLKVREDGDLHHAIDATVIAAITPKFIQQVTKYSQHQEVKNNQALWHDAEIKDAEYAAEAQRMDADLFNKIFNGFPLPWPEFLDELLARISDNPVEMMKSRSWNTYTPIEIAKLKPVFVVRLANHKISGPAHLDTIRSAKLFDEKGIVLSRVSITKLKINKKGQVATGDGIYDPENSNNGDKVVYSAIRQALEAHNGSGELAFPDGYLEYVDHGTKKLVRKVRVAKKVSLPVRLKNKAAADNGSMVRIDVFNTGKKFVFVPIYIKDTVEQVLPNKAIARGKSLWYQITESDQFCFSLYPGDMVHIESKTGIKPKYSNKENNTSVVPIKNFYGYFDGADIATASILVRAHDSSYTARSIGIAGLLKFEKYQVDYFGRYHKVHEKKRQLFVKRDE

SEQ ID NO: 83 - protein, 1688 aa, Ignavibacterium album
MEFKKVLGLDIGTNSIGCALLSLPKSIQDYGKGGRLEWLTSRVIPLDADYMKAFIDGKNGLPQVITPAGKRRQKRGSRRLKHRYKLRRSRLIRVFKTLNWLPEDFPLDNPKRIKETISTEGKFSFRISDYVPISDESYREFYREFGYPENEIEQVIEEINFRRKTKGKNKNPMIKLLPEDWVVYYLRKKALIKPTTKEELIRIIYLFNQRRGFKSSRKDLTETAILDYDEFAKRLAEKEKYSAENYETKFVSITKVKEVVELKTDGRKGKKRFKVILEDSRIEPYEIERKEKPDWEGKEYTFLVTQKLEKGKFKQNKPDLPKEEDWALCTTALDNRMGSKHPGEFFFDELLKAFKEKRGYKIRQYPVNRWRYKKELEFIWTKQCQLNPELNNLNINKEILRKLATVLYPSQSKFFGPKIKEFENSDVLHIISEDIIYYQRDLKSQKSLISECRYEKRKGIDGEIYGLKCIPKSSPLYQEFRIWQDIHNIKVIRKESEVNGKKKINIDETQLYINENIKEKLFELFNSKDSLSEKDILELISLNIINSGIKISKKEEETTHRINLFANRKELKGNETKSRYRKVFKKLGFDGEYILNHPSKLNRLWHSDYSNDYADKEKTEKSILSSLGWKNRNGKWEKSKNYDVFNLPLEVAKAIANLPPLKKEYGSYSALAIRKMLVVMRDGKYWQHPDQIAKDQENTSLMLFDKNLIQLTNNQRKVLNKYLLTLAEVQKRSTLIKQKLNEIEHNPYKLELVSDQDLEKQVLKSFLEKKNESDYLKGLKTYQAGYLIYGKHSEKDVPIVNSPDELGEYIRKKLPNNSLRNPIVEQVIRETIFIVRDVWKSFGIIDEIHIELGRELKNNSEERKKTSESQEKNFQEKERARKLLKELLNSSNFEHYDENGNKIFSSFTVNPNPDSPLDIEKFRIWKNQSGLTDEELNKKLKDEKIPTEIEVKKYILWLTQKCRSPYTGKIIPLSKLFDSNVYEIEHIIPRSKMKNDSTNNLVICELGVNKAKGDRLAANFISESNGKCKFGEVEYTLLKYGDYLQYCKDTFKYQKAKYKNLLATEPPEDFIERQINDTRYIGRKLAELLTPVVKDSKNIIFTIGSITSELKITWGLNGVWKDILRPRFKRLESIINKKLIFQDEDDPNKYHFDLSINPQLDKEGLKRLDHRHHALDATIIAATTREHVRYLNSLNAADNDEEKREYFLSLCNHKIRDFKLPWENFTSEVKSKLLSCVVSYKESKPILSDPFNKYLKWEYKNGKWQKVFAIQIKNDRWKAVRRSMFKEPIGTVWIKKIKEVSLKEAIKIQAIWEEVKNDPVRKKKEKYIYDDYAQKVIAKIVQELGLSSSMRKQDDEKLNKFINEAKVSAGVNKNLNTTNKTIYNLEGRFYEKIKVAEYVLYKAKRMPLNKKEYIEKLSLQKMFNDLPNFILEKSILDNYPEILKELESDNKYIIEPHKKNNPVNRLLLEHILEYHNNPKEAFSTEGLEKLNKKAINKIGKPIKYITRLDGDINEEEIFRGAVFETDKGSNVYFVMYENNQTKDREFLKPNPSISVLKAIEHKNKIDFFAPNRLGFSRIILSPGDLVYVPTNDQYVLIKDNSSNETIINWDDNEFISNRIYQVKKFTGNSCYFLKNDIASLILSYSASNGVGEFGSQNISEYSVDDPPIRIKDVCIKIRVDRLGNVRPL

SEQ ID NO: 84 - protein, 1011 aa, unidentified
MSSKAIDSLEQLDLFKPQEYTLGLDLGIKSIGWAILSGERIANAGVYLFETAEELNSTGNKLISKAAERGRKRRIRRMLDRKARRGRHIRYLLEREGLPTDELEEVVVHQSNRTLWDVRAEAVERKLTKQELAAVLFHLVRHRGYFPNTKKLPPDDESDSADEEQGKINRATSRLREELKASDCKTIGQFLAQNRDRQRNREGDYSNLMARKLVFEEALQILAFQRKQGHELSKDFEKTYLDVLMGQRSGRSPKLGNCSLIPSELRAPSSAPSTEWFKFLQNLGNLQISNAYREEWSIDAPRRAQIIDACSQRSTSSYWQIRRDFQIPDEYRFNLVNYERRDPDVDLQEYLQQQERKTLANFRNWKQLEKIIGTGHPIQTLDEAARLITLIKDDEKLSDQLADLLPEASDKAITQLCELDFTTAAKISLEAMYRILPHMNQGMGFFDACQQESLPEIGVPPAGDRVPPFDEMYNPVVNRVLSQSRKLINAVIDEYGMPAKIRVELARDLGKGRELRERIKLDQLDKSKQNDQRAEDFRAEFQQAPRGDQSLRYRLWKEQNCTCPYSGRMIPVNSVLSEDTQIDHILPISQSFDNSLSNKVLCFTEENAQKSNRTPFEYLDAADFQRLEAISGNWPEAKRNKLLHKSFGKVAEEWKSRALNDTRYLTSALADHLRHHLPDSKIQTVNGRITGYLRKQWGLEKDRDKHTHHAVDAIVVACTTPAIVQQVTLYHQDIRRYKKLGEKRPTPWPETFRQDVLDVEEEIFITRQPKKVSGGIQTKDTLRKHRSKPDRQRVALTKVKLADLERLVEKDASNRNLYEHLKQCLEESGDQPTKAFKAPFYMPSGPEAKQRPILSKVTLLREKPEPPKQLTELSGGRRYDSMAQGRLDIYRYKPGGKRKDEYRVVLQRMIDLMRGEENVHVFQKGVPYDQGPEIEQNYTFLFSLYFDDLVEFQRSADSEVIRGYYRTFNIANGQLKISTYLEGRQDFDFFGANRLAHFAKVQVNLLGKVIK

SEQ ID NO: 85 - protein, 1156 aa, Ruminococcus albus
MGNYYLGLDVGIGSIGWAVINIEKKRIEDFNVRIFKSGEIQEKNRNSRASQQCRRSRGLRRLYRRKSHRKLRLKNYLSIIGLTTSEKIDYYYETADNNVIQLRNKGLSEKLTPEEIAACLIHICNNRGYKDFYEVNVEDIEDPDERNEYKEEHDSIVLISNLMNEGGYCTPAEMICNCREFDEPNSVYRKFHNSAASKNHYLITRHMLVKEVDLILENQSKYYGILDDKTIAKIKDIIFAQRDFEIGPGKNERFRRFTGYLDSIGKCQFFKDQERGSRFTVIADIYAFVNVLSQYTYTNNRGESVFDTSFANDLINSALKNGSMDKRELKAIAKSYHIDISDKNSDTSLTKCFKYIKVVKPLFEKYGYDWDKLIENYTDTDNNVLNRIGIVLSQAQTPKRRREKLKALNIGLDDGLINELTKLKLSGTANVSYKYMQGSIEAFCEGDLYGKYQAKFNKEIPDIDENAKPQKLPPFKNEDDCEFFKNPVVFRSINETRKLINAIIDKYGYPAAVNIETADELNKTFEDRAIDTKRNNDNQKENDRIVKEIIECIKCDEVHARHLIEKYKLWEAQEGKCLYSGETITKEDMLRDKDKLFEVDHIVPYSLILDNTINNKALVYAEENQKKGQRTPLMYMNEAQAADYRVRVNTMFKSKKCSKKKYQYLMLPDLNDQELLGGWRSRNLNDTRYICKYLVNYLRKNLRFDRSYESSDEDDLKIRDHYRVFPVKSRFTSMFRRWWLNEKTWGRYDKAELKKLTYLDHAADAIIIANCRPEYVVLAGEKLKLNKMYHQAGKRITPEYEQSKKACIDNLYKLFRMDRRTAEKLLSGHGRLTPIIPNLSEEVDKRLWDKNIYEQFWKDDKDKKSCEELYRENVASLYKGDPKFASSLSMPVISLKPDHKYRGTITGEEAIRVKEIDGKLIKLKRKSISEITAESINSIYTDDKILIDSLKTIFEQADYKDVGDYLKKTNQHFFTTSSGKRVNKVTVIEKVPSRWLRKEIDDNNFSLLNDSSYYCIELYKDSKGDNNLQGIAMSDIVHDRKTKKLYLKPDFNYPDDYYTHVMYIFPGDYLRIKSTSKKSGEQLKFEGYFISVKNVNENSFRFISDNKPCAKDKRVSITKKDIVIKLAVDLMGKVQGENNGKGISCGEPLSLLKEKN

SEQ ID NO: 86 - protein, 1356 aa, Lactobacillus farciminis
MTKKEQPYNIGLDIGTSSVGWAVTNDNYDLLNIKKKNLWGVRLFEEAQTAKETRLNRSTRRRYRRRKNRINWLNEIFSEELAKTDPSFLIRLQNSWVSKKDPDRKRDKYNLFIDGPYTDKEYYREFPTIFHLRKELILNKDKADIRLIYLALHNILKYRGNFTYEHQKFNISNLNNNLSKELIELNQQLIKYDISFPDDCDWNHISDILIGRGNATQKSSNILKDFTLDKETKKLLKEVINLILGNVAHLNTIFKTSLTKDEEKLNFSGKDIESKLDDLDSILDDDQFTVLDAANRIYSTITLNEILNGESYFSMAKVNQYENHAIDLCKLRDMWHTTKNEEAVEQSRQAYDDYINKPKYGTKELYTSLKKFLKVALPTNLAKEAEEKISKGTYLVKPRNSENGVVPYQLNKIEMEKIIDNQSQYYPFLKENKEKLLSILSFRIPYYVGPLQSAEKNPFAWMERKSNGHARPWNFDEIVDREKSSNKFIRRMTVTDSYLVGEPVLPKNSLIYQRYEVLNELNNIRITENLKTNPIGSRLTVETKQRIYNELFKKYKKVTVKKLTKWLIAQGYYKNPILIGLSQKDEFNSTLTTYLDMKKIFGSSFMEDNKNYDQIEELIEWLTIFEDKQILNEKLHSSKYSYTPDQIKKISNMRYKGWGRLSKKILMDITTETNTPQLLQLSNYSILDLMWATNNNFISIMSNDKYDFKNYIENHNLNKNEDQNISDLVNDIHVSPALKRGITQSIKIVQEIVKFMGHAPKHIFIEVTRETKKSEITTSREKRIKRLQSKLLNKANDFKPQLREYLVPNKKIQEELKKHKNDLSSERIMLYFLQNGKSLYSEESLNINKLSDYQVDHILPRTYIPDDSLENKALVLAKENQRKADDLLLNSNVIDRNLERWTYMLNNNMIGLKKFKNLTRRVITDKDKLGFIHRQLVQTSQMVKGVANILDNMYKNQGTTCIQARANLSTAFRKALSGQDDTYHFKHPELVKNRNVNDFHHAQDAYLASFLGTYRLRRFPTNEMLLMNGEYNKFYGQVKELYSKKKKLPDSRKNGFIISPLVNGTTQYDRNTGEIIWNVGFRDKILKIFNYHQCNVTRKTEIKTGQFYDQTIYSPKNPKYKKLIAQKKDMDPNIYGGFSGDNKSSITIVKIDNNKIKPVAIPIRLINDLKDKKTLQNWLEENVKHKKSIQIIKNNVPIGQIIYSKKVGLLSLNSDREVANRQQLILPPEHSALLRLLQIPDEDLDQILAFYDKNILVEILQELITKMKKFYPFYKGEREFLIANIENFNQATTSEKVNSLEELITLLHANSTSAHLIFNNIEKKAFGRKTHGLTLNNTDFIYQSVTGLYETRIHIE

SEQ ID NO: 87 - protein, 1096 aa, Eubacterium dolichum
MMEVFMGRLVLGLDIGITSVGFGIIDLDESEIVDYGVRLFKEGTAAENETRRTKRGGRRLKRRRVTRREDMLHLLKQAGIISTSFHPLNNPYDVRVKGLNERLNGEELATALLHLCKHRGSSVETIEDDEAKAKEAGETKKVLSMNDQLLKSGKYVCEIQKERLRTNGHIRGHENNFKTRAYVDEAFQILSHQDLSNELKSAIITIISRKRMYYDGPGGPLSPTPYGRYTYFGQKEPIDLIEKMRGKCSLFPNEPRAPKLAYSAELFNLLNDLNNLSIEGEKLTSEQKAMILKIVHEKGKITPKQLAKEVGVSLEQIRGFRIDTKGSPLLSELTGYKMIREVLEKSNDEHLEDHVFYDEIAEILTKTKDIEGRKKQISELSSDLNEESVHQLAGLTKFTAYHSLSFKALRLINEEMLKTELNQMQSITLFGLKQNNELSVKGMKNIQADDTAILSPVAKRAQRETFKVVNRLREIYGEFDSIVVEMAREKNSEEQRKAIRERQKFFEMRNKQVADIIGDDRKINAKLREKLVLYQEQDGKTAYSLEPIDLKLLIDDPNAYEVDHIIPISISLDDSITNKVLVTHRENQEKGNLTPISAFVKGRFTKGSLAQYKAYCLKLKEKNIKTNKGYRKKVEQYLLNENDIYKYDIQKEFINRNLVDTSYASRVVLNTLTTYFKQNEIPTKVFTVKGSLTNAFRRKINLKKDRDEDYGHHAIDALIIASMPKMRLLSTIFSRYKIEDIYDESTGEVFSSGDDSMYYDDRYFAFIASLKAIKVRKFSHKIDTKPNRSVADETIYSTRVIDGKEKVVKKYKDIYDPKFTALAEDILNNAYQEKYLMALHDPQTFDQIVKVVNYYFEEMSKSEKYFTKDKKGRIKISGMNPLSLYRDEHGMLKKYSKKGDGPAITQMKYFDGVLGNHIDISAHYQVRDKKVVLQQISPYRTDFYYSKENGYKFVTIRYKDVRWSEKKKKYVIDQQDYAMKKAEKKIDDTYEFQFSMHRDELIGITKAEGEALIYPDETWHNFNFFFHAGETPEILKFTATNNDKSNKIEVKPIHCYCKMRLMPTISKKIVRIDKYATDVVGNLYKVKKNTLKFEFD

SEQ ID NO: 88 - protein, 1132 aa, Nitratifractor salsuginis
MKKILGVDLGITSFGYAILQETGKDLYRCLDNSVVMRNNPYDEKSGESSQSIRSTQKSMRRLIEKRKKRIRCVAQTMERYGILDYSETMKINDPKNNPIKNRWQLRAVDAWKRPLSPQELFAIFAHMAKHRGYKSIATEDLIYELELELGLNDPEKESEKKADERRQVYNALRHLEELRKKYGGETIAQTIHRAVEAGDLRSYRNHDDYEKMIRREDIEEEIEKVLLRQAELGALGLPEEQVSELIDELKACITDQEMPTIDESLFGKCTFYKDELAAPAYSYLYDLYRLYKKLADLNIDGYEVTQEDREKVIEWVEKKIAQGKNLKKITHKDLRKILGLAPEQKIFGVEDERIVKGKKEPRTFVPFFFLADIAKFKELFASIQKHPDALQIFRELAEILQRSKTPQEALDRLRALMAGKGIDTDDRELLELFKNKRSGTRELSHRYILEALPLFLEGYDEKEVQRILGFDDREDYSRYPKSLRHLHLREGNLFEKEENPINNHAVKSLASWALGLIADLSWRYGPFDEIILETTRDALPEKIRKEIDKAMREREKALDKIIGKYKKEFPSIDKRLARKIQLWERQKGLDLYSGKVINLSQLLDGSADIEHIVPQSLGGLSTDYNTIVTLKSVNAAKGNRLPGDWLAGNPDYRERIGMLSEKGLIDWKKRKNLLAQSLDEIYTENTHSKGIRATSYLEALVAQVLKRYYPFPDPELRKNGIGVRMIPGKVTSKTRSLLGIKSKSRETNFHHAEDALILSTLTRGWQNRLHRMLRDNYGKSEAELKELWKKYMPHIEGLTLADYIDEAFRRFMSKGEESLFYRDMFDTIRSISYWVDKKPLSASSHKETVYSSRHEVPTLRKNILEAFDSLNVIKDRHKLTTEEFMKRYDKEIRQKLWLHRIGNTNDESYRAVEERATQIAQILTRYQLMDAQNDKEIDEKFQQALKELITSPIEVTGKLLRKMRFVYDKLNAMQIDRGLVETDKNMLGIHISKGPNEKLIFRRMDVNNAHELQKERSGILCYLNEMLFIFNKKGLIHYGCLRSYLEKGQGSKYIALFNPRFPANPKAQPSKFTSDSKIKQVGIGSATGIIKAHLDLDGHVRSYEVFGTLPEGSIEWFKEESGYGRVEDDPHH

SEQ ID NO: 89 - protein, 1173 aa, Rhodospirillum rubrum
MRPIEPWILGLDIGTDSLGWAVFSCEEKGPPTAKELLGGGVRLFDSGRDAKDHTSRQAERGAFRRARRQTRTWPWRRDRLIALFQAAGLTPPAAETRQIALALRREAVSRPLAPDALWAALLHLAHHRGFRSNRIDKRERAAAKALAKAKPAKATAKATAPAKEADDEAGFWEGAEAALRQRMAASGAPTVGALLADDLDRGQPVRMRYNQSDRDGVVAPTRALIAEELAEIVARQSSAYPGLDWPAVTRLVLDQRPLRSKGAGPCAFLPGEDRALRALPTVQDFIIRQTLANLRLPSTSADEPRPLTDEEHAKALALLSTARFVEWPALRRALGLKRGVKFTAETERNGAKQAARGTAGNLTEAILAPLIPGWSGWDLDRKDRVFSDLWAARQDRSALLALIGDPRGPTRVTEDETAEAVADAIQIVLPTGRASLSAKAARAIAQAMAPGIGYDEAVTLALGLHHSHRPRQERLARLPYYAAALPDVGLDGDPVGPPPAEDDGAAAEAYYGRIGNISVHIALNETRKIVNALLHRHGPILRLVMVETTRELKAGADERKRMIAEQAERERENAEIDVELRKSDRWMANARERRQRVRLARRQNNLCPYTSTPIGHADLLGDAYDIDHVIPLARGGRDSLDNMVLCQSDANKTKGDKTPWEAFHDKPGWIAQRDDFLARLDPQTAKALAWRFADDAGERVARKSAEDEDQGFLPRQLTDTGYIARVALRYLSLVTNEPNAVVATNGRLTGLLRLAWDITPGPAPRDLLPTPRDALRDDTAARRFLDGLTPPPLAKAVEGAVQARLAALGRSRVADAGLADALGLTLASLGGGGKNRADHRHHFIDAAMIAVTTRGLINQINQASGAGRILDLRKWPRTNFEPPYPTFRAEVMKQWDHIHPSIRPAHRDGGSLHAATVFGVRNRPDARVLVQRKPVEKLFLDANAKPLPADKIAEIIDGFASPRMAKRFKALLARYQAAHPEVPPALAALAVARDPAFGPRGMTANTVIAGRSDGDGEDAGLITPFRANPKAAVRTMGNAVYEVWEIQVKGRPRWTHRVLTRFDRTQPAPPPPPENARLVMRLRRGDLVYWPLESGDRLFLVKKMAVDGRLALWPARLATGKATALYAQLSCPNINLNGDQGYCVQSAEGIRKEKIRTTSCTALGRLRLSKKAT

SEQ ID NO: 90 - protein, 1348 aa, Finegoldia magna
MKSEKKYYIGLDVGTNSVGWAVTDEFYNILRAKGKDLWGVRLFEKADTAANTRIFRSGRRRNDRKGMRLQILREIFEDEIKKVDKDFYDRLDESKFWAEDKKVSGKYSLFNDKNFSDKQYFEKFPTIFHLRKYLMEEHGKVDIRYYFLAINQMMKRRGHFLIDGQISHVTDDKPLKEQLILLINDLLKIELEEELMDSIFEILADVNEKRTDKKNNLKELIKGQDFNKQEGNILNSIFESIVTGKAKIKNIISDEDILEKIKEDNKEDFVLTGDSYEENLQYFEEVLQENITLFNTLKSTYDFLILQSILKGKSTLSDAQVERYDEHKKDLEILKKVIKKYDEDGKLFKQVFKEDNGNGYVSYIGYYLNKNKKITAKKKISNIEFTKYVKGILEKQCDCEDEDVKYLLGKIEQENFLLKQISSINSVIPHQIHLFELDKILENLAKNYPSFNNKKEEFTKIEKIRKTFTFRIPYYVGPLNDYHKNNGGNAWIFRNKGEKIRPWNFEKIVDLHKSEEEFIKRMLNQCTYLPEETVLPKSSILYSEYMVLNELNNLRINGKPLDTDVKLKLIEELFKKKTKVTLKSIRDYMVRNNFADKEDFDNSEKNLEIASNMKSYIDFNNILEDKFDVEMVEDLIEKITIHTGNKKLLKKYIEETYPDLSSSQIQKIINLKYKDWGRLSRKLLDGIKGTKKETEKTDTVINFLRNSSDNLMQIIGSQNYSFNEYIDKLRKKYIPQEISYEVVENLYVSPSVKKMIWQVIRVTEEITKVMGYDPDKIFIEMAKSEEEKKTTISRKNKLLDLYKAIKKDERDSQYEKLLTGLNKLDDSDLRSRKLYLYYTQMGRDMYTGEKIDLDKLFDSTHYDKDHIIPQSMKKDDSIINNLVLVNKNANQTTKGNIYPVPSSIRNNPKIYNYWKYLMEKEFISKEKYNRLIRNTPLTNEELGGFINRQLVETRQSTKAIKELFEKFYQKSKIIPVKASLASDLRKDMNTLKSREVNDLHHAHDAFLNIVAGDVWNREFTSNPINYVKENREGDKVKYSLSKDFTRPRKSKGKVIWTPEKGRKLIVDTLNKPSVLISNESHVKKGELFNATIAGKKDYKKGKIYLPLKKDDRLQDVSKYGGYKAINGAFFFLVEHTKSKKRIRSIELFPLHLLSKFYEDKNTVLDYAINVLQLQDPKIIIDKINYRTEIIIDNFSYLISTKSNDGSITVKPNEQMYWRVDEISNLKKIENKYKKDAILTEEDRKIMESYIDKIYQQFKAGKYKNRRTTDTIIEKYEIIDLDTLDNKQLYQLLVAFISLSYKTSNNAVDFTVIGLGTECGKPRITNLPDNTYLVYKSITGIYEKRIRIK

SEQ ID NO: 91 - protein, 1114 aa, Eubacterium rectale
MNYTEKEKLFMKYILALDIGIASVGWAILDKESETVIEAGSNIFPEASAADNQLRRDMRGAKRNNRRLKTRINDFIKLWENNNLSIPQFKSTEIVGLKVRAITEEITLDELYLILYSYLKHRGISYLEDALDDTVSGSSAYANGLKLNAKELETHYPCEIQQERLNTIGKYRGQSQIINENGEVLDLSNVFTIGAYRKEIQRVFEIQKKYHPELTDEFCDGYMLIFNRKRKYYEGPGNEKSRTDYGRFTTKLDANGNYITEDNIFEKLIGKCSVYPDELRAAAASYTAQEYNVLNDLNNLTINGRKLEENEKHEIVERIKSSNTINMRKIISDCMGENIDDFAGARIDKSGKEIFHKFEVYNKMRKALLEIGIDISNYSREELDEIGYIMTINTDKEAMMEAFQKSWIDLSDDVKQCLINMRKTNGALFNKWQSFSLKIMNELIPEMYAQPKEQMTLLTEMGVTKGTQEEFAGLKYIPVDVVSEDIFNPVVRRSVRISFKILNAVLKKYKALDTIVIEMPRDRNSEEQKKRINDSQKLNEKEMEYIEKKLAVTYGIKLSPSDFSSQKQLSLKLKLWNEQDGICLYSGKTIDPNDIINNPQLFEIDHIIPRSISFDDARSNKVLVYRSENQKKGNQTPYYYLTHSHSEWSFEQYKATVMNLSKKKEYAISRKKIQNLLYSEDITKMDVLKGFINRNINDTSYASRLVLNTIQNFFMANEADTKVKVIKGSYTHQMRCNLKLDKNRDESYSHHAVDAMLIGYSELGYEAYHKLQGEFIDFETGEILRKDMWDENMSDEVYADYLYGKKWANIRNEVVKAEKNVKYWHYVMRKSNRGLCNQTIRGTREYDGKQYKINKLDIRTKEGIKVFAKLAFSKKDSDRERLLVYLNDRRTFDDLCKIYEDYSDAANPFVQYEKETGDIIRKYSKKHNGPRIDKLKYKDGEVGACIDISHKYGFEKGSKKVILESLVPYRMDVYYKEENHSYYLVGVKQSDIKFEKGRNVIDEEAYARILVNEKMIQPGQSRADLENLGFKFKLSFYKNDIIEYEKDGKIYTERLVSRTMPKQRNYIETKPIDKAKFEKQNLVGLGKTKFIKKYRYDILGNKYSCSEEKFTSFC

SEQ ID NO: 92 - protein, 1084 aa, Corynebacterium diphtheriae
MKYHVGIDVGTFSVGLAAIEVDDAGMPIKTLSLVSHIHDSGLDPDKIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDEPSDAFKAIREEIKRASGQPVPETATVGQMVTLCELGTLKLRGEGGVLSARLQQSDHAREIQEICRMQEIGQELYRKIIDVVFAAESPKGSASSRVGKDPLQPGKNRALKASDAFQRYRIAALIGNLRVRVDGEKRILSVEEKNLVFDHLVNLAPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLADGVDLYTARLQEFGIEPSWTPPAPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLEFLDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHEETIGKLSKVKLGSQLSVSDIDKASSEALWCALTREPDFDPKDGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFGDTRLRLRPLQMSKEGIKKESAPELSKIIDRPGWLPAVNKLFSEGNVTVVRRDSLGRVRLESTAHLPVTWKVQ

SEQ ID NO: 93 - protein, 1152 aa, Roseburia inulinivorans
MNAEHGKEGLLIMEENFQYRIGLDIGITSVGWAVLQNNSQDEPVRITDLGVRIFDVAENPKNGDALAAPRRDARTTRRRLRRRRHRLERIKFLLQENGLIEMDSFMERYYKGNLPDVYQLRYEGLDRKLKDEELAQVLIHIAKHRGFRSTRKAETKEKEGGAVLKATTENQKIMQEKGYRTVGEMLYLDEAFHTECLWNEKGYVLTPRNRPDDYKHTILRSMLVEEVHAIFAAQRAHGNQKATEGLEEAYVEIMTSQRSFDMGPGLQPDGKPSPYAMEGFGDRVGKCTFEKDEYRAPKATYTAELFVALQKINHTKLIDEFGTGRFFSEEERKTIIGLLLSSKELKYGTIRKKLNIDPSLKFNSLNYSAKKEGETEEERVLDTEKAKFASMFWTYEYSKCLKDRTEEMPVGEKADLFDRIGEILTAYKNDDSRSSRLKELGLSGEEIDGLLDLSPAKYQRVSLKAMRKMQPYLEDGLIYDKACEAAGYDFRALNDGNKKHLLKGEEINAIVNDITNPVVKRSVSQTIKVINAIIQKYGSPQAVNIELAREMSKNFQDRTNLEKEMKKRQQENERAKQQIIELGKQNPTGQDILKYRLWNDQGGYCLYSGKKIPLEELFDGGYDIDHILPYSITFDDSYRNKVLVTAQENRQKGNRTPYEYFGADEKRWEDYEASVRLLVRDYKKQQKLLKKNFTEEERKEFKERNLNDTKYITRVVYNMIRQNLELEPFNHPEKKKQVWAVNGAVTSYLRKRWGLMQKDRSTDRHHAMDAVVIACCTDGMIHKISRYMQGRELAYSRNFKFPDEETGEILNRDNFTREQWDEKFGVKVPLPWNSFRDELDIRLLNEDPKNFLLTHADVQRELDYPGWMYGEEESPIEEGRYINYIRPLFVSRMPNHKVTGSAHDATIRSARDYETRGVVITKVPLTDLKLNKDNEIEGYYDKDSDRLLYQALVRQLLLHGNDGKKAFAEDFHKPKADGTEGPVVRKVKIEKKQTSGVMVRGGTGIAANGEMVRIDVFRENGKYYFVPVYTADVVRKVLPNRAATHTKPYSEWRVMDDANFVFSLYSRDLIHVKSKKDIKTNLVNGGLLLQKEIFAYYTGADIATASIAGFANDSNFKFRGLGIQSLEIFEKCQVDILGNISVVRHENRQEFH

SEQ ID NO: 94 - protein, 1029 aa, Alicycliphilus denitrificans
MRSLRYRLALDLGSTSLGWALFRLDACNRPTAVIKAGVRIFSDGRNPKDGSSLAVTRRAARAMRRRRDRLLKRKTRMQAKLVEHGFFPADAGKRKALEQLNPYALRAKGLQEALLPGEFARALFHINQRRGFKSNRKTDKKDNDSGVLKKAIGQLRQQMAEQGSRTVGEYLWTRLQQGQGVRARYREKPYTTEEGKKRIDKSYDLYIDRAMIEQEFDALWAAQAAFNPTLFHEAARADLKDTLLHQRPLRPVKPGRCTLLPEEERAPLALPSTQRFRIHQEVNHLRLLDENLREVALTLAQRDAVVTALETKAKLSFEQIRKLLKLSGSVQFNLEDAKRTELKGNATSAALARKELFGAAWSGFDEALQDEIVWQLVTEEGEGALIAWLQTHTGVDEARAQAIVDVSLPEGYGNLSRKALARIVPALRAAVITYDKAVQAAGFDHHSQLGFEYDASEVEDLVHPETGEIRSVFKQLPYYGKALQRHVAFGSGKPEDPDEKRYGKIANPTVHIGLNQVRMVVNALIRRYGRPTEVVIELARDLKQSREQKVEAQRRQADNQRRNARIRRSIAEVLGIGEERVRGSDIQKWICWEELSFDAADRRCPYSGVQISAAMLLSDEVEVEHILPFSKTLDDSLNNRTVAMRQANRIKRNRTPWDARAEFEAQGWSYEDILQRAERMPLRKRYRFAPDGYERWLGDDKDFLARALNDTRYLSRVAAEYLRLVCPGTRVIPGQLTALLRGKFGLNDVLGLDGEKNRNDHRHHAVDACVIGVTDQGLMQRFATASAQARGDGLTRLVDGMPMPWPTYRDHVERAVRHIWVSHRPDHGFEGAMMEETSYGIRKDGSIKQRRKADGSAGREISNLIRIHEATQPLRHGVSADGQPLAYKGYVGGSNYCIEITVNDKGKWEGEVISTFRAYGVVRAGGMGRLRNPHEGQNGRKLIMRLVIGDSVRLEVDGAERTMRIVKISGSNGQIFMAPIHEANVDARNTDKQDAFTYTSKYAGSLQKAKTRRVTISPIGEVRDPGFKG

SEQ ID NO: 95 - protein, 1179 aa, Sphaerochaeta globosa
MSKKVSRRYEEQAQEICQRLGSRPYSIGLDLGVGSIGVAVAAYDPIKKQPSDLVFVSSRIFIPSTGAAERRQKRGQRNSLRHRANRLKFLWKLLAERNLMLSYSEQDVPDPARLRFEDAVVRANPYELRLKGLNEQLTLSELGYALYHIANHRGSSSVRTFLDEEKSSDDKKLEEQQAMTEQLAKEKGISTFIEVLTAFNTNGLIGYRNSESVKSKGVPVPTRDIISNEIDVLLQTQKQFYQEILSDEYCDRIVSAILFENEKIVPEAGCCPYFPDEKKLPRCHFLNEERRLWEAINNARIKMPMQEGAAKRYQSASFSDEQRHILFHIARSGTDITPKLVQKEFPALKTSIIVLQGKEKAIQKIAGFRFRRLEEKSFWKRLSEEQKDDFFSAWTNTPDDKRLSKYLMKHLLLTENEVVDALKTVSLIGDYGPIGKTATQLLMKHLEDGLTYTEALERGMETGEFQELSVWEQQSLLPYYGQILTGSTQALMGKYWHSAFKEKRDSEGFFKPNTNSDEEKYGRIANPVVHQTLNELRKLMNELITILGAKPQEITVELARELKVGAEKREDIIKQQTKQEKEAVLAYSKYCEPNNLDKRYIERFRLLEDQAFVCPYCLEHISVADIAAGRADVDHIFPRDDTADNSYGNKVVAHRQCNDIKGKRTPYAAFSNTSAWGPIMHYLDETPGMWRKRRKFETNEEEYAKYLQSKGFVSRFESDNSYIAKAAKEYLRCLFNPNNVTAVGSLKGMETSILRKAWNLQGIDDLLGSRHWSKDADTSPTMRKNRDDNRHHGLDAIVALYCSRSLVQMINTMSEQGKRAVEIEAMIPIPGYASEPNLSFEAQRELFRKKILEFMDLHAFVSMKTDNDANGALLKDTVYSILGADTQGEDLVFVVKKKIKDIGVKIGDYEEVASAIRGRITDKQPKWYPMEMKDKIEQLQSKNEAALQKYKESLVQAAAVLEESNRKLIESGKKPIQLSEKTISKKALELVGGYYYLISNNKRTKTFVVKEPSNEVKGFAFDTGSNLCLDFYHDAQGKLCGEIIRKIQAMNPSYKPAYMKQGYSLYVRLYQGDVCELRASDLTEAESNLAKTTHVRLPNAKPGRTFVIIITFTEMGSGYQIYFSNLAKSKKGQDTSFTLTTIKNYDVRKVQLSSAGLVRYVSPLLVDKIEKDEVALCGE

SEQ ID NO: 96 - protein, 1374 aa, Fusobacterium nucleatum
MKKQKFSDYYLGFDIGTNSVGWCVTDLDYNVLRFNKKDMWGSRLFDEAKTAAERRVQRNSRRRLKRRKWRLNLLEEIFSDEIMKIDSNFFRRLKESSLWLEDKNSKEKFTLFNDDNYKDYDFYKQYPTIFHLRDELIKNPEKKDIRLIYLALHSIFKSRGHFLFEGQNLKEIKNFETLYNNLISFLEDNGINKSIDKDNIEKLEKIICDSGKGLKDKEKEFKGIFNSDKQLVAIFKLSVGSSVSLNDLFDTDEYKKEEVEKEKISFREQIYEDDKPIYYSILGEKIELLDIAKSFYDFMVLNNILSDSNYISEAKVKLYEEHKKDLKNLKYIIRKYNKENYDKLFKDKNENNYPAYIGLNKEKDKKEVVEKSRLKIDDLIKVIKGYLPKPERIEEKDKTIFNEILNKIELKTILPKQRISDNGTLPYQIHEVELEKILENQSKYYDFLNYEENGVSTKDKLLKTFKFRIPYYVGPLNSYHKDKGGNSWIVRKEEGKILPWNFEQKVDIEKSAEEFIKRMTNKCTYLNGEDVIPKDSFLYSEYIILNELNKVQVNDEFLNEENKRKIIDELFKENKKVSEKKFKEYLLVNQIANRTVELKGIKDSFNSNYVSYIKFKDIFGEKLNLDIYKEISEKSILWKCLYGDDKKIFEKKIKNEYGDILNKDEIKKINSFKFNTWGRLSEKLLTGIEFINLETGECYSSVMEALRRTNYNLMELLSSKFTLQESIDNENKEMNEVSYRDLIEESYVSPSLKRAILQTLKIYEEIKKITGRVPKKVFIEMARGGDESMKNKKIPARQEQLKKLYDSCGNDIANFSIDIKEMKNSLSSYDNNSLRQKKLYLYYLQFGKCMYTGREIDLDRLLQNNDTYDIDHIYPRSKVIKDDSFDNLVLVLKNENAEKSNEYPVKKEIQEKMKSFWRFLKEKNFISDEKYKRLTGKDDFELRGFMARQLVNVRQTTKEVGKILQQIEPEIKIVYSKAEIASSFREMFDFIKVRELNDTHHAKDAYLNIVAGNVYNTKFTEKPYRYLQEIKENYDVKKIYNYDIKNAWDKENSLEIVKKNMEKNTVNITRFIKEEKGELFNLNPIKKGETSNEIISIKPKLYDGKDNKLNEKYGYYTSLKAAYFIYVEHEKKNKKVKTFERITRIDSTLIKNEKNLIKYLVSQKKLLNPKIIKKIYKEQTLIIDSYPYTFTGVDSNKKVELKNKKQLYLEKKYEQILKNALKFVEDNQGETEENYKFIYLKKRNNNEKNETIDAVKERYNIEFNEMYDKFLEKLSSKDYKNYINNKLYTNFLNSKEKFKKLKLWEKSLILREFLKIFNKNTYGKYEIKDSQTKEKLFSFPEDTGRIRLGQSSLGNNKELLEESVTGLFVKKIKL

SEQ ID NO: 97 - protein, 1056 aa, Pasteurella multocida
MQTTNLSYILGLDLGIASVGWAVVEINENEDPIGLIDVGVRIFERAEVPKTGESLALSRRLARSTRRLIRRRAHRLLLAKRFLKREGILSTIDLEKGLPNQAWELRVAGLERRLSAIEWGAVLLHLIKHRGYLSKRKNESQTNNKELGALLSGVAQNHQLLQSDDYRTPAELALKKFAKEEGHIRNQRGAYTHTFNRLDLLAELNLLFAQQHQFGNPHCKEHIQQYMTELLMWQKPALSGEAILKMLGKCTHEKNEFKAAKHTYSAERFVWLTKLNNLRILEDGAERALNEEERQLLINHPYEKSKLTYAQVRKLLGLSEQAIFKHLRYSKENAESATFMELKAWHAIRKALENQGLKDTWQDLAKKPDLLDEIGTAFSLYKTDEDIQQYLTNKVPNSVINALLVSLNFDKFIELSLKSLRKILPLMEQGKRYDQACREIYGHHYGEANQKTSQLLPAIPAQEIRNPVVLRTLSQARKVINAIIRQYGSPARVHIETGRELGKSFKERREIQKQQEDNRTKRESAVQKFKELFSDFSSEPKSKDILKFRLYEQQHGKCLYSGKEINIHRLNEKGYVEIDHALPFSRTWDDSFNNKVLVLASENQNKGNQTPYEWLQGKINSERWKNFVALVLGSQCSAAKKQRLLTQVIDDNKFIDRNLNDTRYIARFLSNYIQENLLLVGKNKKNVFTPNGQITALLRSRWGLIKARENNNRHHALDAIVVACATPSMQQKITRFIRFKEVHPYKIENRYEMVDQESGEIISPHFPEPWAYFRQEVNIRVFDNHPDTVLKEMLPDRPQANHQFVQPLFVSRAPTRKMSGQGHMETIKSAKRLAEGISVLRIPLTQLKPNLLENMVNKEREPALYAGLKARLAEFNQDPAKAFATPFYKQGGQQVKAIRVEQVQKSGVLVRENNGVADNASIVRTDVFIKNNKFFLVPIYTWQVAKGILPNKAIVAHKNEDEWEEMDEGAKFKFSLFPNDLVELKTKKEYFFGYYIGLDRATGNISLKEHDGEISKGKDGVYRVGVKLALSFEKYQVDELGKNRQICRPQQRQPVR

SEQ ID NO: 98 - protein, 1113 aa, Alcanivorax pacificus
MRYRVGLDLGTASVGAAVFSMDEQGNPMELIWHYERLFSEPLVPDMGQLKPKKAARRLARQQRRQIDRRASRLRRIAIVSRRLGIAPGRNDSGVHGNDVPTLRAMAVNERIELGQLRAVLLRMGKKRGYGGTFKAVRKVGEAGEVASGASRLEEEMVALASVQNKDSVTVGEYLAARVEHGLPSKLKVAANNEYYAPEYALFRQYLGLPAIKGRPDCLPNMYALRHQIEHEFERIWATQSQFHDVMKDHGVKEEIRNAIFFQRPLKSPADKVGRCSLQTNLPRAPRAQIAAQNFRIEKQMADLRWGMGRRAEMLNDHQKAVIRELLNQQKELSFRKIYKELERAGCPGPEGKGLNMDRAALGGRDDLSGNTTLAAWRKLGLEDRWQELDEVTQIQVINFLADLGSPEQLDTDDWSCRFMGKNGRPRNFSDEFVAFMNELRMTDGFDRLSKMGFEGGRSSYSIKALKALTEWMIAPHWRETPETHRVDEEAAIRECYPESLATPAQGGRQSKLEPPPLTGNEVVDVALRQVRHTINMMIDDLGSVPAQIVVEMAREMKGGVTRRNDIEKQNKRFASERKKAAQSIEENGKTPTPARILRYQLWIEQGHQCPYCESNISLEQALSGAYTNFEHILPRTLTQIGRKRSELVLAHRECNDEKGNRTPYQAFGHDDRRWRIVEQRANALPKKSSRKTRLLLLKDFEGEALTDESIDEFADRQLHESSWLAKVTTQWLSSLGSDVYVSRGSLTAELRRRWGLDTVIPQVRFESGMPVVDEEGAEITPEEFEKFRLQWEGHRVTREMRTDRRPDKRIDHRHHLVDAIVTALTSRSLYQQYAKAWKVADEKQRHGRVDVKVELPMPILTIRDIALEAVRSVRISHKPDRYPDGRFFEATAYGIAQRLDERSGEKVDWLVSRKSLTDLAPEKKSIDVDKVRANISRIVGEAIRLHISNIFEKRVSKGMTPQQALREPIEFQGNILRKVRCFYSKADDCVRIEHSSRRGHHYKMLLNDGFAYMEVPCKEGILYGVPNLVRPSEAVGIKRAPESGDFIRFYKGDTVKNIKTGRVYTIKQILGDGGGKLILTPVTETKPADLLSAKWGRLKVGGRNIHLLRLCAE

SEQ ID NO: 99 - protein, 1236 aa, Mycoplasma mobile
MYFYKNKENKLNKKVVLGLDLGIASVGWCLTDISQKEDNKFPIILHGVRLFETVDDSDDKLLNETRRKKRGQRRRNRRLFTRKRDFIKYLIDNNIIELEFDKNPKILVRNFIEKYINPFSKNLELKYKSVTNLPIGFHNLRKAAINEKYKLDKSELIVLLYFYLSLRGAFFDNPEDTKSKEMNKNEIEIFDKNESIKNAEFPIDKIIEFYKISGKIRSTINLKFGHQDYLKEIKQVFEKQNIDFMNYEKFAMEEKSFFSRIRNYSEGPGNEKSFSKYGLYANENGNPELIINEKGQKIYTKIFKTLWESKIGKCSYDKKLYRAPKNSFSAKVFDITNKLTDWKHKNEYISERLKRKILLSRFLNKDSKSAVEKILKEENIKFENLSEIAYNKDDNKINLPIINAYHSLTTIFKKHLINFENYLISNENDLSKLMSFYKQQSEKLFVPNEKGSYEINQNNNVLHIFDAISNILNKFSTIQDRIRILEGYFEFSNLKKDVKSSEIYSEIAKLREFSGTSSLSFGAYYKFIPNLISEGSKNYSTISYEEKALQNQKNNFSHSNLFEKTWVEDLIASPTVKRSLRQTMNLLKEIFKYSEKNNLEIEKIVVEVTRSSNNKHERKKIEGINKYRKEKYEELKKVYDLPNENTTLLKKLWLLRQQQGYDAYSLRKIEANDVINKPWNYDIDHIVPRSISFDDSFSNLVIVNKLDNAKKSNDLSAKQFIEKIYGIEKLKEAKENWGNWYLRNANGKAFNDKGKFIKLYTIDNLDEFDNSDFINRNLSDTSYITNALVNHLTFSNSKYKYSVVSVNGKQTSNLRNQIAFVGIKNNKETEREWKRPEGFKSINSNDFLIREEGKNDVKDDVLIKDRSFNGHHAEDAYFITIISQYFRSFKRIERLNVNYRKETRELDDLEKNNIKFKEKASFDNFLLINALDELNEKLNQMRFSRMVITKKNTQLFNETLYSGKYDKGKNTIKKVEKLNLLDNRTDKIKKIEEFFDEDKLKENELTKLHIFNHDKNLYETLKIIWNEVKIEIKNKNLNEKNYFKYFVNKKLQEGKISFNEWVPILDNDFKIIRKIRYIKFSSEEKETDEIIFSQSNFLKIDQRQNFSFHNTLYWVQIWVYKNQKDQYCFISIDARNSKFEKDEIKINYEKLKTQKEKLQIINEEPILKINKGDLFENEEKELFYIVGRDEKPQKLEIKYILGKKIKDQKQIQKPVKKYFPNWKKVNLTYMGEIFKK

SEQ ID NO: 100 - protein, 1397 aa, unidentified
MTKNYISPIAIDLGAKFTGVALYQYLEGADCTQEVAKGLLVDDRGNVTWSQEGRRGKRHQVRGYKRRKMAKRLLWLILDSEYGIKREEVTEPLLKFINGLLNRRGYTYISEEVDEESMNVSPLPFSEMMPDYFNSSAPLLEQLAKLLSDKNKLVRFRAEGKIPSNKNEFKKLLDTALDGKYKDEKKELSEAWGNILIASENVLKSTVDGHKSRSEYLANIKEDIKSNEELEKQISSKEIDGFYNLVGHLSNFQLRLLRKYFNDPNMSGVSYWDEKRLEKYFYQWVQGWHTKGGTDEAEKKNIILKTKGAPLLKTLKSLSADLTIPPYEDQNNRRPPKCQSVLLSDEKLTMHYPKWKEWVGQLVKQNDNAYLNENVTLANALHRIVERSRSIDPYQLRLLISITDAEKRNDLAGYKRLKLSLGSEVDEFLLLVKNIVDETKEAREGLWFETENKLFFKCGKTPPRKEKLKSTLLSAVLGKNLSDDEQSSFIEEFWKSGTPKIERRNVRGWCRLASQVQKTYGVYLKEYGLQQLHKLEAGKKLDDKPLALLYKNSGLIASKIGEALNIEPDEVSRFASPHSLAQIFNIIEGDVAGFNKTCRACTYENIWRMQEEKVESLLTNQLLSEIHGERKVPLKSAMCTRLSADSTRPFDGQMASIIEHIARKIAQHKIAQINDVPKEFSIDIPIIIESNQFSFTAELEEIKRGRGSAKAKKAKELGEKSKAGWVSKTERIKTSSEGICPYTGAPLGGSGEIDHIIPRSLTGRTKKTVFNSEANLIYCSSKGNHDKGNRVYVIEQLNDKYLKKQFSTSDVNLIKKKIKTTIQRFTEGGEKLRSFSELSREDQKAFRHALFVPELKSEVTSLLAVKNITRVNGTQAWLAKKIASLLAEHLDKQGRDYTLSAHQIDPWSVSKQRKMLASAEPIWAKKDPQPAASHVVDAVCTFLEALEQPHTASRLKTISSTSFEKTGWRSALIPDLIKVDALDRRPKYRRYNIGSTSLFKDGIYAERFLPILIDENGLMAGYDIDNSLKAKGADVVFESLSPFLLFKGEEVGAQSLSDWQERIDGRYLYMSIDKVKAFDYLQEKVGEKDIAAELLNSIHFTQRKTELRAKFSDDSGKKMKTLDAIRKSLKLTVTVNEIGKRKEKCGFSGTIGIPAKSAWENLLDEPLLETYWGTKMPPQEIWEKVYRKHFPRNIPNQAHRKVRKDFSLPVVDSVSGGFRVKRKTPNGYNYQLLAIDGYSAVGFKKEGDNVDFKSPALVPQIAESKSVTPISSELVHLDKNEIVYFDEWRKIDISDSDLKQFVSSLELAPGSQNRFYIRFTVDEDQFERHFKSALRVNGIQDLDTVNKTFDWNREIPSLLIPPRSNLFLLETGQKITFEYIANGANAEVKKAYSLRRA

SEQ ID NO: 101 - protein, 1333 aa, Planococcus antarcticus
MKNYTIGLDIGVASVGWVCIDENYKILNYNNRHAFGVHEFESAESAAGRRLKRGMRRRYNRRKKRLQLLQSLFDSYITDSGFFSKTDSQHFWKNNNEFENRSLTEVLSSLRISSRKYPTIYHLRSDLIESNKKMDLRLVYLALHNLVKYRGHFLQEGNWSEAASAEGMDDQLLELVTRYAELENLSPLDLSESQWKAAETLLLNRNLTKTDQSKELTAMFGKEYEPFCKLVAGLGVSLHQLFPSSEQALAYKETKTKVQLSNENVEEVMELLLEEESALLEAVQPFYQQVVLYELLKGETYVAKAKVSAFKQYQKDMASLKNLLDKTFGEKVYRSYFISDKNSQREYQKSHKVEVLCKLDQFNKEAKFAETFYKDLKKLLEDKSKTSIGTTEKDEMLRIIKAIDSNQFLQKQKGIQNAAIPHQNSLYEAEKILRNQQAHYPFITTEWIEKVKQILAFRIPYYIGPLVKDTTQSPFSWVERKGDAPITPWNFDEQIDKAASAEAFISRMRKTCTYLKGQEVLPKSSLTYERFEVLNELNGIQLRTTGAESDFRHRLSYEMKCWIIDNVFKQYKTVSTKRLLQELKKSPYADELYDEHTGEIKEVFGTQKENAFATSLSGYISMKSILGAVVDDNPAMTEELIYWIAVFEDREILHLKIQEKYPSITDVQRQKLALVKLPGWGRFSRLLIDGLPLDEQGQSVLDHMEQYSSVFMEVLKNKGFGLEKKIQKMNQHQVDGTKKIRYEDIEELAGSPALKRGIWRSVKIVEELVSIFGEPANIVLEVAREDGEKKRTKSRKDQWEELTKTTLKNDPDLKSFIGEIKSQGDQRFNEQRFWLYVTQQGKCLYTGKALDIQNLSMYEVDHILPQNFVKDDSLDNLALVMPEANQRKNQVGQNKMPLEIIEANQQYAMRTLWERLHELKLISSGKLGRLKKPSFDEVDKDKFIARQLVETRQIIKHVRDLLDERFSKSDIHLVKAGIVSKFRRFSEIPKIRDYNNKHHAMDALFAAALIQSILGKYGKNFLAFDLSKKDRQKQWRSVKGSNKEFFLFKNFGNLRLQSPVTGEEVSGVEYMKHVYFELPWQTTKMTQTGDGMFYKESIFSPKVKQAKYVSPKTEKFVHDEVKNHSICLVEFTFMKKEKEVQETKFIDLKVIEHHQFLKEPESQLAKFLAEKETNSPIIHARIIRTIPKYQKIWIEHFPYYFISTRELHNARQFEISYELMEKVKQLSERSSVEELKIVFGLLIDQMNDNYPIYTKSSIQDRVQKFVDTQLYDFKSFEIGFEELKKAVAANAQRSDTFGSRISKKPKPEEVAIGYESITGLKYRKPRSVVGTKR

SEQ ID NO: 102 - protein, 1424 aa, Prevotella sp.
MTQKVLGLDLGTNSIGSAVRNLDLSDDLQWQLEFFSSDIFRSSVNKESNGREYSLAAQRSAHRRSRGLNEVRRRRLWATLNLLIKHGFCPMSSESLMRWCTYDKRKGLFREYPIDDKDFNAWILLDFNGDGRPDYSSPYQLRRELVTRQFDFEQPIERYKLGRALYHIAQHRGFKSSKGETLSQQETNSKPSSTDEIPDVAGAMKASEEKLSKGLSTYMKEHNLLTVGAAFAQLEDEGVRVRNNNDYRAIRSQFQHEIETIFKFQQGLSVESELYERLISEKKNVGTIFYKRPLRSQRGNVGKCTLERSKPRCAIGHPLFEKFRAWTLINNIKVRMSVDTLDEQLPMKLRLDLYNECFLAFVRTEFKFEDIRKYLEKRLGIHFSYNDKTINYKDSTSVAGCPITARFRKMLGEEWESFRVEGQKERQAHSKNNISFHRVSYSIEDIWHFCYDAEEPEAVLAFAQETLRLERKKAEELVRIWSAMPQGYAMLSQKAIRNINKILMLGLKYSDAVILAKVPELVDVSDEELLSIAKDYYLVEAQVNYDKRINSIVNGLIAKYKSVSEEYRFADHNYEYLLDESDEKDIIRQIENSLGARRWSLMDANEQTDILQKVRDRYQDFFRSHERKFVESPKLGESFENYLTKKFPMVEREQWKKLYHPSQITIYRPVSVGKDRSVLRLGNPDIGAIKNPTVLRVLNTLRRRVNQLLDDGVISPDETRVVVETARELNDANRKWALDTYNRIRHDENEKIKKILEEFYPKRDGISTDDIDKARYVIDQREVDYFTGSKTYNKDIKKYKFWLEQGGQCMYTGRTINLSNLFDPNAFDIEHTIPESLSFDSSDMNLTLCDAHYNRFIKKNHIPTDMPNYDKAITIDGKEYPAITSQLQRWVERVERLNRNVEYWKGQARRAQNKDRKDQCMREMHLWKMELEYWKKKLERFTVTEVTDGFKNSQLVDTRVITRHAVLYLKSIFPHVDVQRGDVTAKFRKILGIQSVDEKKDRSLHSHHAIDATTLTIIPVSAKRDRMLELFAKIEEINKMLSFSGSEDRTGLIQELEGLKNKLQMEVKVCRIGHNVSEIGTFINDNIIVNHHIKNQALTPVRRRLRKKGYIVGGVDNPRWQTGDALRGEIHKASYYGAITQFAKDDEGKVLMKEGRPQVNPTIKFVIRRELKYKKSAADSGFASWDDLGKAIVDKELFALMKGQFPAETSFKDACEQGIYMIKKGKNGMPDIKLHHIRHVRCEAPQSGLKIKEQTYKSEKEYKRYFYAAVGDLYAMCCYTNGKIREFRIYSLYDVSCHRKSDIEDIPEFITDKKGNRLMLDYKLRTGDMILLYKDNPAELYDLDNVNLSRRLYKINRFESQSNLVLMTHHLSTSKERGRSLGKTVDYQNLPESIRSSVKSLNFLIMGENRDFVIKNGKIIFNHR

SEQ ID NO: 103 - protein, 1146 aa, Alicyclobacillus hesperidum
MAYRLGLDIGITSVGWAVVALEKDESGLKPVRIQDLGVRIFDKAEDSKTGASLALPRREARSARRRTRRRRHRLWRVKRLLEQHGILSMEQIEALYAQRTSSPDVYALRVAGLDRCLIAEEIARVLIHIAHRRGFQSNRKSEIKDSDAGKLLKAVQENENLMQSKGYRTVAEMLVSEATKTDAEGKLVHGKKHGYVSNVRNKAGEYRHTVSRQAIVDEVRKIFAAQRALGNDVMSEELEDSYLKILCSQRNFDDGPGGDSPYGHGSVSPDGVRQSIYERMVGSCTFETGEKRAPRSSYSFERFQLLTKVVNLRIYRQQEDGGRYPCELTQTERARVIDCAYEQTKITYGKLRKLLDMKDTESFAGLTYGLNRSRNKTEDTVFVEMKFYHEVRKALQRAGVFIQDLSIETLDQIGWILSVWKSDDNRRKKLSTLGLSDNVIEELLPLNGSKFGHLSLKAIRKILPFLEDGYSYDVACELAGYQFQGKTEYVKQRLLPPLGEGEVTNPVVRRALSQAIKVVNAVIRKHGSPESIHIELARELSKNLDERRKIEKAQKENQKNNEQIKDEIREILGSAHVTGRDIVKYKLFKQQQEFCMYSGEKLDVTRLFEPGYAEVDHIIPYGISFDDSYDNKVLVKTEQNRQKGNRTPLEYLRDKPEQKAKFIALVESIPLSQKKKNHLLMDKRAIDLEQEGFRERNLSDTRYITRALMNHIQAWLLFDETASTRSKRVVCVNGAVTAYMRARWGLTKDRDAGDKHHAADAVVVACIGDSLIQRVTKYDKFKRNALADRNRYVQQVSKSEGITQYVDKETGEVFTWESFDERKFLPNEPLEPWPFFRDELLARLSDDPSKNIRAIGLLTYSETEQIDPIFVSRMPTRKVTGAAHKETIRSPRIVKVDDNKGTEIQVVVSKVALTELKLTKDGEIKDYFRPEDDPRLYNTLRERLVQFGGDAKAAFKEPVYKISKDGSVRTPVRKVKIQEKLTLGVPVHGGRGIAENGGMVRIDVFAKGGKYYFVPIYVADVLKRELPNRLATAHKPYSEWRVVDDSYQFKFSLYPNDAVMIKPSREVDITYKDRKEPVGCRIMYFVSANIASASISLRTHDNSGELEGLGIQGLEVFEKYVVGPLGDTHPVYKERRMPFRVERKMN

SEQ ID NO: 104 - protein, 1363 aa, Lactobacillus rhamnosus
MTKLNQPYGIGLDIGSNSIGFAVVDANSHLLRLKGETAIGARLFREGQSAADRRGSRTTRRRLSRTRWRLSFLRDFFAPHITKIDPDFFLRQKYSEISPKDKDRFKYEKRLFNDRTDAEFYEDYPSMYHLRLHLMTHTHKADPREIFLAIHHILKSRGHFLTPGAAKDFNTDKVDLEDIFPALTEAYAQVYPDLELTFDLAKADDFKAKLLDEQATPSDTQKALVNLLLSSDGEKEIVKKRKQVLTEFAKAITGLKTKFNLALGTEVDEADASNWQFSMGQLDDKWSNIETSMTDQGTEIFEQIQELYRARLLNGIVPAGMSLSQAKVADYGQHKEDLELFKTYLKKLNDHELAKTIRGLYDRYINGDDAKPFLREDFVKALTKEVTAHPNEVSEQLLNRMGQANFMLKQRTKANGAIPIQLQQRELDQIIANQSKYYDWLAAPNPVEAHRWKMPYQLDELLNFHIPYYVGPLITPKQQAESGENVFAWMVRKDPSGNITPYNFDEKVDREASANTFIQRMKTTDTYLIGEDVLPKQSLLYQKYEVLNELNNVRINNECLGTDQKQRLIREVFERHSSVTIKQVADNLVAHGDFARRPEIRGLADEKRFLSSLSTYHQLKEILHEAIDDPTKLLDIENIITWSTVFEDHTIFETKLAEIEWLDPKKINELSGIRYRGWGQFSRKLLDGLKLGNGHTVIQELMLSNHNLMQILADETLKETMTELNQDKLKTDDIEDVINDAYTSPSNKKALRQVLRVVEDIKHAANGQDPSWLFIETADGTGTAGKRTQSRQKQIQTVYANAAQELIDSAVRGELEDKIADKASFTDRLVLYFMQGGRDIYTGAPLNIDQLSHYDIDHILPQSLIKDDSLDNRVLVNATINREKNNVFASTLFAGKMKATWRKWHEAGLISGRKLRNLMLRPDEIDKFAKGFVARQLVETRQIIKLTEQIAAAQYPNTKIIAVKAGLSHQLREELDFPKNRDVNHYHHAFDAFLAARIGTYLLKRYPKLAPFFTYGEFAKVDVKKFREFNFIGALTHAKKNIIAKDTGEIVWDKERDIRELDRIYNFKRMLITHEVYFETADLFKQTIYAAKDSKERGGSKQLIPKKQGYPTQVYGGYTQESGSYNALVRVAEADTTAYQVIKISAQNASKIASANLKSREKGKQLLNEIVVKQLAKRRKNWKPSANSFKIVIPRFGMGTLFQNAKYGLFMVNSDTYYRNYQELWLSRENQKLLKKLFSIKYEKTQMNHDALQVYKAIIDQVEKFFKLYDINQFRAKLSDAIERFEKLPINTDGNKIGKTETLRQILIGLQANGTRSNVKNLGIKTDLGLLQVGSGIKLDKDTQIVYQSPSGLFKRRIPLADL

SEQ ID NO: 105 - protein, 1150 aa, Enterococcus faecalis
MYSIGLDLGISSVGWSVIDERTGNVIDLGVRLFSAKNSEKNLERRTNRGGRRLIRRKTNRLKDAKKILAAVGFYEDKSLKNSCPYQLRVKGLTEPLSRGEIYKVTLHILKKRGISYLDEVDTEAAKESQDYKEQVRKNAQLLTKYTPGQIQLQRLKENNRVKTGINAQGNYQLNVFKVSAYANELATILKTQQAFYPNELTDDWIALFVQPGIAEEAGLIYRKRPYYHGPGNEANNSPYGRWSDFQKTGEPATNIFDKLIGKDFQGELRASGLSLSAQQYNLLNDLTNLKIDGEVPLSSEQKEYILTELMTKEFTRFGVNDVVKLLGVKKERLSGWRLDKKGKPEIHTLKGYRNWRKIFAEAGIDLATLPTETIDCLAKVLTLNTEREGIENTLAFELPELSESVKLLVLDRYKELSQSISTQSWHRFSLKTLHLLIPELMNATSEQNTLLEQFQLKSDVRKRYSEYKKLPTKDVLAEIYNPTVNKTVSQAFKVIDALLVKYGKEQIRYITIEMPRDDNEEDEKKRIKELHAKNSQRKNDSQSYFMQKSGWSQEKFQTTIQKNRRFLAKLLYYYEQDGICAYTGLPISPELLVSDSTEIDHIIPISISLDDSINNKVLVLSKANQVKGQQTPYDAWMDGSFKKINGKFSNWDDYQKWVESRHFSHKKENNLLETRNIFDSEQVEKFLARNLNDTRYASRLVLNTLQSFFTNQETKVRVVNGSFTHTLRKKWGADLDKTRETHHHHAVDATLCAVTSFVKVSRYHYAVKEETGEKVMREIDFETGEIVNEMSYWEFKKSKKYERKTYQVKWPNFREQLKPVNLHPRIKFSHQVDRKANRKLSDATIYSVREKTEVKTLKSGKQKITTDEYTIGKIKDIYTLDGWEAFKKKQDKLLMKDLDEKTYERLLSIAETTPDFQEVEEKNGKVKRVKRSPFAVYCEENDIPAIQKYAKKNNGPLIRSLKYYDGKLNKHINITKDSQGRPVEKTKNGRKVTLQSLKPYRYDIYQDLETKAYYTVQLYYSDLRFVEGKYGITEKEYMKKVAEQTKGQVVRFCFSLQKNDGLEIEWKDSQRYDVRFYNFQSANSINFKGLEQEMMPAENQFKQKPYNNGAINLNIAKYGKEGKKLRKFNTDILGKKHYLFYEKEPKNIIK

SEQ ID NO: 106 - protein, 1035 aa, unidentified
MRRLGLDLGTNSIGWCLLDLGDDGEPVSIFRTGARIFSDGRDPKSLGSLKATRREARLTRRRRDRFIQRQKNLINALVKYGLMPADEIQRQALAYKDPYPIRKKALDEAIDPYEMGRAIFHINQRRGFKSNRKSADNEAGVVKQSIADLEMKLGEAGARTIGEFLADRQATNDTVRARRLSGTNALYEFYPDRYMLEQEFDTLWAKQAAFNPSLYIEAARERLKEIVFFQRKLKPQEVGRCIFLSDEDRISKALPSFQRFRIYQELSNLAWIDHDGVAHRITASLALRDHLFDELEHKKKLTFKAMRAILRKQGVVDYPVGFNLESDNRDHLIGNLTSCIMRDAKKMIGSAWDRLDEEEQDSFILMLQDDQKGDDEVRSILTQQYGLSDDVAEDCLDVRLPDGHGSLSKKAIDRILPVLRDQGLIYYDAVKEAGLGEANLYDPYAALSDKLDYYGKALAGHVMGASGKFEDSDEKRYGTISNPTVHIALNQVRAVVNELIRLHGKPDEVVIEIGRDLPMGADGKRELERFQKEGRAKNERARDELKKLGHIDSRESRQKFQLWEQLAKEPVDRCCPFTGKMMSISDLFSDKVEIEHLLPFSLTLDDSMANKTVCFRQANRDKGNRAPFDAFGNSPAGYDWQEILGRSQNLPYAKRWRFLPDAMKRFEADGGFLERQLNDTRYISRYTTEYISTIIPKNKIWVVTGRLTSLLRGFWGLNSILRGHNTDDGTPAKKSRDDHRHHAIDAIVVGMTSRGLLQKVSKAARRSEDLDLTRLFEGRIDPWDGFRDEVKKHIDAIIVSHRPRKKSQGALHNDTAYGIVEHAENGASTVVHRVPITSLGKQSDIEKVRDPLIKSALLNETAGLSGKSFENAVQKWCADNSIKSLRIVETVSIIPITDKEGVAYKGYKGDGNAYMDIYQDPTSSKWKGEIVSRFDANQKGFIPSWQSQFPTARLIMRLRINDLLKLQDGEIEEIYRVQRLSGSKILMAPHTEANVDARDRDKNDTFKLTSKSPGKLQSASARKVHISPTGLIREG

SEQ ID NO: 107 - protein, 1389 aa, Oenococcus kitaharae
MARDYSVGLDIGTSSVGWAAIDNKYHLIRAKSKNLIGVRLFDSAVTAEKRRGYRTTRRRLSRRHWRLRLLNDIFAGPLTDFGDENFLARLKYSWVHPQDQSNQAHFAAGLLFDSKEQDKDFYRKYPTIYHLRLALMNDDQKHDLREVYLAIHHLVKYRGHFLIEGDVKADSAFDVHTFADAIQRYAESNNSDENLLGKIDEKKLSAALTDKHGSKSQRAETAETAFDILDLQSKKQIQAILKSVVGNQANLMAIFGLDSSAISKDEQKNYKFSFDDADIDEKIADSEALLSDTEFEFLCDLKAAFDGLTLKMLLGDDKTVSAAMVRRFNEHQKDWEYIKSHIRNAKNAGNGLYEKSKKFDGINAAYLALQSDNEDDRKKAKKIFQDEISSADIPDDVKADFLKKIDDDQFLPIQRTKNNGTIPHQLHRNELEQIIEKQGIYYPFLKDTYQENSHELNKITALINFRVPYYVGPLVEEEQKIADDGKNIPDPTNHWMVRKSNDTITPWNLSQVVDLDKSGRRFIERLTGTDTYLIGEPTLPKNSLLYQKFDVLQELNNIRVSGRRLDIRAKQDAFEHLFKVQKTVSATNLKDFLVQAGYISEDTQIEGLADVNGKNFNNALTTYNYLVSVLGREFVENPSNEELLEEITELQTVFEDKKVLRRQLDQLDGLSDHNREKLSRKHYTGWGRISKKLLTTKIVQNADKIDNQTFDVPRMNQSIIDTLYNTKMNLMEIINNAEDDFGVRAWIDKQNTTDGDEQDVYSLIDELAGPKEIKRGIVQSFRILDDITKAVGYAPKRVYLEFARKTQESHLTNSRKNQLSTLLKNAGLSELVTQVSQYDAAALQNDRLYLYFLQQGKDMYSGEKLNLDNLSNYDIDHIIPQAYTKDNSLDNRVLVSNITNRRKSDSSNYLPALIDKMRPFWSVLSKQGLLSKHKFANLTRTRDFDDMEKERFIARSLVETRQIIKNVASLIDSHFGGETKAVAIRSSLTADMRRYVDIPKNRDINDYHHAFDALLFSTVGQYTENSGLMKKGQLSDSAGNQYNRYIKEWIHAARLNAQSQRVNPFGFVVGSMRNAAPGKLNPETGEITPEENADWSIADLDYLHKVMNFRKITVTRRLKDQKGQLYDESRYPSVLHDAKSKASINFDKHKPVDLYGGFSSAKPAYAALIKFKNKFRLVNVLRQWTYSDKNSEDYILEQIRGKYPKAEMVLSHIPYGQLVKKDGALVTISSATELHNFEQLWLPLADYKLINTLLKTKEDNLVDILHNRLDLPEMTIESAFYKAFDSILSFAFNRYALHQNALVKLQAHRDDFNALNYEDKQQTLERILDALHASPASSDLKKINLSSGFGRLFSPSHFTLADTDEFIFQSVTGLFSTQKTVAQLYQETK

SEQ ID NO: 108 - protein, 1024 aa, Helicobacter mustelae
MIRTLGIDIGIASIGWAVIEGEYTDKGLENKEIVASGVRVFTKAENPKNKESLALPRTLARSARRRNARKKGRIQQVKHYLSKALGLDLECFVQGEKLATLFQTSKDFLSPWELRERALYRVLDKEELARVILHIAKRRGYDDITYGVEDNDSGKIKKAIAENSKRIKEEQCKTIGEMMYKLYFQKSLNVRNKKESYNRCVGRSELREELKTIFQIQQELKSPWVNEELIYKLLGNPDAQSKQEREGLIFYQRPLKGFGDKIGKCSHIKKGENSPYRACKHAPSAEEFVALTKSINFLKNLTNRHGLCFSQEDMCVYLGKILQEAQKNEKGLTYSKLKLLLDLPSDFEFLGLDYSGKNPEKAVFLSLPSTFKLNKITQDRKTQDKIANILGANKDWEAILKELESLQLSKEQIQTIKDAKLNFSKHINLSLEALYHLLPLMREGKRYDEGVEILQERGIFSKPQPKNRQLLPPLSELAKEESYFDIPNPVLRRALSEFRKVVNALLEKYGGFHYFHIELTRDVCKAKSARMQLEKINKKNKSENDAASQLLEVLGLPNTYNNRLKCKLWKQQEEYCLYSGEKITIDHLKDQRALQIDHAFPLSRSLDDSQSNKVLCLTSSNQEKSNKTPYEWLGSDEKKWDMYVGRVYSSNFSPSKKRKLTQKNFKERNEEDFLARNLVDTGYIGRVTKEYIKHSLSFLPLPDGKKEHIRIISGSMTSTMRSFWGVQEKNRDHHLHHAQDAIIIACIEPSMIQKYTTYLKDKETHRLKSHQKAQILREGDHKLSLRWPMSNFKDKIQESIQNIIPSHHVSHKVTGELHQETVRTKEFYYQAFGGEEGVKKALKFGKIREINQGIVDNGAMVRVDIFKSKDKGKFYAVPIYTYDFAIGKLPNKAIVQGKKNGIIKDWLEMDENYEFCFSLFKNDCIKIQTKEMQEAVLAIYKSTNSAKATIELEHLSKYALKNEDEEKMFTDTDKEKNKTMTRESCGIQGLKVFQKVKLSVLGEVLEHKPRNRQNIALKTTPKHV

SEQ ID NO: 109 - protein, 1064 aa, Bradyrhizobium sp.
MKRTSLRAYRLGVDLGANSLGWFVVWLDDHGQPEGLGPGGVRIFPDGRNPQSKQSNAAGRRLARSARRRRDRYLQRRGKLMGLLVKHGLMPADEPARKRLECLDPYGLRAKALDEVLPLHHVGRALFHLNQRRGLFANRAIEQGDKDASAIKAAAGRLQTSMQACGARTLGEFLNRRHQLRATVRARSPVGGDVQARYEFYPTRAMVDAEFEAIWAAQAPHHPTMTAEAHDTIREAIFSQRAMKRPSIGKCSLDPATSQDDVDGFRCAWSHPLAQRFRIWQDVRNLAVVETGPTSSRLGKEDQDKVARALLQTDQLSFDEIRGLLGLPSDARFNLESDRRDHLKGDATGAILSARRHFGPAWHDRSLDRQIDIVALLESALDEAAIIASLGTTHSLDEAAAQRALSALLPDGYCRLGLRAIKRVLPLMEAGRTYAEAASAAGYDHALLPGGKLSPTGYLPYYGQWLQNDVVGSDDERDTNERRWGRLPNPTVHIGIGQLRRVVNELIRWHGPPAEITVELTRDLKLSPRRLAELEREQAENQRKNDKRTSLLRKLGLPASTHNLLKLRLWDEQGDVASECPYTGEAIGLERLVSDDVDIDHLIPFSISWDDSAANKVVCMRYANREKGNRTPFEAFGHRQGRPYDWADIAERAARLPRGKRWRFGPGARAQFEELGDFQARLLNETSWLARVAKQYLAAVTHPHRIHVLPGRLTALLRATWELNDLLPGSDDRAAKSRKDHRHHAIDALVAALTDQALLRRMANAHDDTRRKIEVLLPWPTFRIDLETRLKAMLVSHKPDHGLQARLHEDTAYGTVEHPETEDGANLVYRKTFVDISEKEIDRIRDRRLRDLVRAHVAGERQQGKTLKAAVLSFAQRRDIAGHPNGIRHVRLTKSIKPDYLVPIRDKAGRIYKSYNAGENAFVDILQAESGRWIARATTVFQANQANESHDAPAAQPIMRVFKGDMLRIDHAGAEKFVKIVRLSPSNNLLYLVEHHQAGVFQTRHDDPEDSFRWLFASFDKLREWNAELVRIDTLGQPWRRKRGLETGSEDATRIGWTRPKKWP

SEQ ID NO: 110 - protein, 1358 aa, Acidaminococcus sp.
MGKMYYLGLDIGTNSVGYAVTDPSYHLLKFKGEPMWGAHVFAAGNQSAERRSFRTSRRRLDRRQQRVKLVQEIFAPVISPIDPRFFIRLHESALWRDDVAETDKHIFFNDPTYTDKEYYSDYPTIHHLIVDLMESSEKHDPRLVYLAVAWLVAHRGHFLNEVDKDNIGDVLSFDAFYPEFLAFLSDNGVSPWVCESKALQATLLSRNSVNDKYKALKSLIFGSQKPEDNFDANISEDGLIQLLAGKKVKVNKLFPQESNDASFTLNDKEDAIEEILGTLTPDECEWIAHIRRLFDWAIMKHALKDGRTISESKVKLYEQHHHDLTQLKYFVKTYLAKEYDDIFRNVDSETTKNYVAYSYHVKEVKGTLPKNKATQEEFCKYVLGKVKNIECSEADKVDFDEMIQRLTDNSFMPKQVSGENRVIPYQLYYYELKTILNKAASYLPFLTQCGKDAISNQDKLLSIMTFRIPYFVGPLRKDNSEHAWLERKAGKIYPWNFNDKVDLDKSEEAFIRRMTNTCTYYPGEDVLPLDSLIYEKFMILNEINNIRIDGYPISVDVKQQVFGLFEKKRRVTVKDIQNLLLSLGALDKHGKLTGIDTTIHSNYNTYHHFKSLMERGVLTRDDVERIVERMTYSDDTKRVRLWLNNNYGTLTADDVKHISRLRKHDFGRLSKMFLTGLKGVHKETGERASILDFMWNTNDNLMQLLSECYTFSDEITKLQEAYYAKAQLSLNDFLDSMYISNAVKRPIYRTLAVVNDIRKACGTAPKRIFIEMARDGESKKKRSVTRREQIKNLYRSIRKDFQQEVDFLEKILENKSDGQLQSDALYLYFAQLGRDMYTGDPIKLEHIKDQSFYNIDHIYPQSMVKDDSLDNKVLVQSEINGEKSSRYPLDAAIRNKMKPLWDAYYNHGLISLKKYQRLTRSTPFTDDEKWDFINRQLVETRQSTKALAILLKRKFPDTEIVYSKAGLSSDFRHEFGLVKSRNINDLHHAKDAFLAIVTGNVYHERFNRRWFMVNQPYSVKTKTLFTHSIKNGNFVAWNGEEDLGRIVKMLKQNKNTIHFTRFSFDRKEGLFDIQPLKASTGLVPRKAGLDVVKYGGYDKSTAAYYLLVRFTLEDKKTQHKLMMIPVEGLYKARIDHDKEFLTDYAQTTISEILQKDKQKVINIMFPMGTRHIKLNSMISIDGFYLSIGGKSSKGKSVLCHAMVPLIVPHKIECYIKAMESFARKFKENNKLRIVEKFDKITVEDNLNLYELFLQKLQHNPYNKFFSTQFDVLTNGRSTFTKLSPEEQVQTLLNILSIFKTCRSSGCDLKSINGSAQAARIMISADLTGLSKKYSDIRLVEQSASGLFVSKSQNLLEYL

SEQ ID NO: 111 - protein, 1082 aa, Methylosinus trichosporium
MRVLGLDAGIASLGWALIEIEESNRGELSQGTIIGAGTWMFDAPEEKTQAGAKLKSEQRRTFRGQRRVVRRRRQRMNEVRRILHSHGLLPSSDRDALKQPGLDPWRIRAEALDRLLGPVELAVALGHIARHRGFKSNSKGAKTNDPADDTSKMKRAVNETREKLARFGSAAKMLVEDESFVLRQTPTKNGASEIVRRFRNREGDYSRSLLRDDLAAEMRALFTAQARFQSAIATADLQTAFTKAAFFQRPLQDSEKLVGPCPFEVDEKRAPKRGYSFELFRFLSRLNHVTLRDGKQERTLTRDELALAAADFGAAAKVSFTALRKKLKLPETTVFVGVKADEESKLDVVARSGKAAEGTARLRSVIVDALGELAWGALLCSPEKLDKIAEVISFRSDIGRISEGLAQAGCNAPLVDALTAAASDGRFDPFTGAGHISSKAARNILSGLRQGMTYDKACCAADYDHTASRERGAFDVGGHGREALKRILQEERISRELVGSPTARKALIESIKQVKAIVERYGVPDRIHVELARDVGKSIEEREEITRGIEKRNRQKDKLRGLFEKEVGRPPQDGARGKEELLRFELWSEQMGRCLYTDDYISPSQLVATDDAVQVDHILPWSRFADDSYANKTLCMAKANQDKKGRTPYEWFKAEKTDTEWDAFIVRVEALADMKGFKKRNYKLRNAEEAAAKFRNRNLNDTRWACRLLAEALKQLYPKGEKDKDGKERRRVFSRPGALTDRLRRAWGLQWMKKSTKGDRIPDDRHHALDAIVIAATTESLLQRATREVQEIEDKGLHYDLVKNVTPPWPGFREQAVEAVEKVFVARAERRRARGKAHDATIRHIAVREGEQRVYERRKVAELKLADLDRVKDAERNARLIEKLRNWIEAGSPKDDPPLSPKGDPIFKVRLVTKSKVNIALDTGNPKRPGTVDRGEMARVDVFRKASKKGKYEYYLVPIYPHDIATMKTPPIRAVQAYKPEDEWPEMDSSYEFCWSLVPMTYLQVISSKGEIFEGYYRGMNRSVGAIQLSAHSNSSDVVQGIGARTLTEFKKFNVDRFGRKHEVERELRTWRGETWRGKAYI

SEQ ID NO: 112 - protein, 1105 aa, Actinomyces coleocanis
MDNKNYRIGIDVGLNSIGFCAVEVDQHDTPLGFLNLSVYRHDAGIDPNGKKTNTTRLAMSGVARRTRRLFRKRKRRLAALDRFIEAQGWTLPDHADYKDPYTPWLVRAELAQTPIRDENDLHEKLAIAVRHIARHRGWRSPWVPVRSLHVEQPPSDQYLALKERVEAKTLLQMPEGATPAEMVVALDLSVDVNLRPKNREKTDTRPENKKPGFLGGKLMQSDNANELRKIAKIQGLDDALLRELIELVFAADSPKGASGELVGYDVLPGQHGKRRAEKAHPAFQRYRIASIVSNLRIRHLGSGADERLDVETQKRVFEYLLNAKPTADITWSDVAEEIGVERNLLMGTATQTADGERASAKPPVDVTNVAFATCKIKPLKEWWLNADYEARCVMVSALSHAEKLTEGTAAEVEVAEFLQNLSDEDNEKLDSFSLPIGRAAYSVDSLERLTKRMIENGEDLFEARVNEFGVSEDWRPPAEPIGARVGNPAVDRVLKAVNRYLMAAEAEWGAPLSVNIEHVREGFISKRQAVEIDRENQKRYQRNQAVRSQIADHINATSGVRGSDVTRYLAIQRQNGECLYCGTAITFVNSEMDHIVPRAGLGSTNTRDNLVATCERCNKSKSNKPFAVWAAECGIPGVSVAEALKRVDFWIADGFASSKEHRELQKGVKDRLKRKVSDPEIDNRSMESVAWMARELAHRVQYYFDEKHTGTKVRVFRGSLTSAARKASGFESRVNFIGGNGKTRLDRRHHAMDAATVAMLRNSVAKTLVLRGNIRASERAIGAAETWKSFRGENVADRQIFESWSENMRVLVEKFNLALYNDEVSIFSSLRLQLGNGKAHDDTITKLQMHKVGDAWSLTEIDRASTPALWCALTRQPDFTWKDGLPANEDRTIIVNGTHYGPLDKVGIFGKAAASLLVRGGSVDIGSAIHHARIYRIAGKKPTYGMVRVFAPDLLRYRNEDLFNVELPPQSVSMRYAEPKVREAIREGKAEYLGWLVVGDELLLDLSSETSGQIAELQQDFPGTTHWTVAGFFSPSRLRLRPVYLAQEGLGEDVSEGSKSIIAGQGWRPAVNKVFGSAMPEVIRRDGLGRKRRFSYSGLPVSWQG

SEQ ID NO: 113 - protein, 1442 aa, unidentified
MPVLSPLSPNAAQGRRRWSLALDIGEGSIGWAVAEVDAEGRVLQLTGTGVTLFPSAWSNENGTYVAHGAADRAVRGQQQRHDSRRRRLAGLARLCAPVLERSPEDLKDLTRTPPKADPRAIFFLRADAARRPLDGPELFRVLHHMAAHRGIRLAELQEVDPPPESDADDAAPAATEDEDGTRRAAADERAFRRLMAEHMHRHGTQPTCGEIMAGRLRETPAGAQPVTRARDGLRVGGGVAVPTRALIEQEFDAIRAIQAPRHPDLPWDSLRRLVLDQAPIAVPPATPCLFLEELRRRGETFQGRTITREAIDRGLTVDPLIQALRIRETVGNLRLHERITEPDGRQRYVPRAMPELGLSHGELTAPERDTLVRALMHDPDGLAAKDGRIPYTRLRKLIGYDNSPVCFAQERDTSGGGITVNPTDPLMARWIDGWVDLPLKARSLYVRDVVARGADSAALARLLAEGAHGVPPVAAAAVPAATAAILESDIMQPGRYSVCPWAAEAILDAWANAPTEGFYDVTRGLFGFAPGEIVLEDLRRARGALLAHLPRTMAAARTPNRAAQQRGPLPAYESVIPSQLITSLRRAHKGRAADWSAADPEERNPFLRTWTGNAATDHILNQVRKTANEVITKYGNRRGWDPLPSRITVELAREAKHGVIRRNEIAKENRENEGRRKKESAALDTFCQDNTVSWQAGGLPKERAALRLRLAQRQEFFCPYCAERPKLRATDLFSPAETEIDHVIERRMGGDGPDNLVLAHKDCNNAKGKKTPHEHAGDLLDSPALAALWQGWRKENADRLKGKGHKARTPREDKDFMDRVGWRFEEDARAKAEENQERRGRRMLHDTARATRLARLYLAAAVMPEDPAEIGAPPVETPPSPEDPTGYTAIYRTISRVQPVNGSVTHMLRQRLLQRDKNRDYQTHHAEDACLLLLAGPAVVQAFNTEAAQHGADAPDDRPVDLMPTSDAYHQQRRARALGRVPLATVDAALADIVMPESDRQDPETGRVHWRLTRAGRGLKRRIDDLTRNCVILSRPRRPSETGTPGALHNATHYGRREITVDGRTDTVVTQRMNARDLVALLDNAKIVPAARLDAAAPGDTILKEICTEIADRHDRVVDPEGTHARRWISARLAALVPAHAEAVARDIAELADLDALADADRTPEQEARRSALRQSPYLGRAISAKKADGRARAREQEILTRALLDPHWGPRGLRHLIMREARAPSLVRIRANKTDAFGRPVPDAAVWVKTDGNAVSQLWRLTSVVTDDGRRIPLPKPIEKRIEISNLEYARLNGLDEGAGVTGNNAPPRPLRQDIDRLTPLWRDHGTAPGGYLGTAVGELEDKARSALRGKAMRQTLTDAGITAEAGWRLDSEGAVCDLEVAKGDTVKKDGKTYKVGVITQGIFGMPVDAAGSAPRTPEDCEKFEEQYGIKPWKAKGIPLA

SEQ ID NO: 114 - protein, 1384 aa, Coriobacterium glomerans
MKLRGIEDDYSIGLDMGTSSVGWAVTDERGTLAHFKRKPTWGSRLFREAQTAAVARMPRGQRRRYVRRRWRLDLLQKLFEQQMEQADPDFFIRLRQSRLLRDDRAEEHADYRWPLFNDCKFTERDYYQRFPTIYHVRSWLMETDEQADIRLIYLALHNIVKHRGNFLREGQSLSAKSARPDEALNHLRETLRVWSSERGFECSIADNGSILAMLTHPDLSPSDRRKKIAPLFDVKSDDAAADKKLGIALAGAVIGLKTEFKNIFGDFPCEDSSIYLSNDEAVDAVRSACPDDCAELFDRLCEVYSAYVLQGLLSYAPGQTISANMVEKYRRYGEDLALLKKLVKIYAPDQYRMFFSGATYPGTGIYDAAQARGYTKYNLGPKKSEYKPSESMQYDDFRKAVEKLFAKTDARADERYRMMMDRFDKQQFLRRLKTSDNGSIYHQLHLEELKAIVENQGRFYPFLKRDADKLVSLVSFRIPYYVGPLSTRNARTDQHGENRFAWSERKPGMQDEPIFPWNWESIIDRSKSAEKFILRMTGMCTYLQQEPVLPKSSLLYEEFCVLNELNGAHWSIDGDDEHRFDAADREGIIEELFRRKRTVSYGDVAGWMERERNQIGAHVCGGQGEKGFESKLGSYIFFCKDVFKVERLEQSDYPMIERIILWNTLFEDRKILSQRLKEEYGSRLSAEQIKTICKKRFTGWGRLSEKFLTGITVQVDEDSVSIMDVLREGCPVSGKRGRAMVMMEILRDEELGFQKKVDDFNRAFFAENAQALGVNELPGSPAVRRSLNQSIRIVDEIASIAGKAPANIFIEVTRDEDPKKKGRRTKRRYNDLKDALEAFKKEDPELWRELCETAPNDMDERLSLYFMQRGKCLYSGRAIDIHQLSNAGIYEVDHIIPRTYVKDDSLENKALVYREENQRKTDMLLIDPEIRRRMSGYWRMLHEAKLIGDKKFRNLLRSRIDDKALKGFIARQLVETGQMVKLVRSLLEARYPETNIISVKASISHDLRTAAELVKCREANDFHHAHDAFLACRVGLFIQKRHPCVYENPIGLSQVVRNYVRQQADIFKRCRTIPGSSGFIVNSFMTSGFDKETGEIFKDDWDAEAEVEGIRRSLNFRQCFISRMPFEDHGVFWDATIYSPRAKKTAALPLKQGLNPSRYGSFSREQFAYFFIYKARNPRKEQTLFEFAQVPVRLSAQIRQDENALERYARELAKDQGLEFIRIERSKILKNQLIEIDGDRLCITGKEEVRNACELAFAQDEMRVIRMLVSEKPVSRECVISLFNRILLHGDQASRRLSKQLKLALLSEAFSEASDNVQRNVVLGLIAIFNGSTNMVNLSDIGGSKFAGNVRIKYKKELASPKVNVHLIDQSVTGMFERRTKIGL

SEQ ID NO: 115 - protein, 4 aa, synthetic construct
AGGG

SEQ ID NO: 116 - protein, 1477 aa, synthetic construct
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 117 - protein, 1403 aa, synthetic construct
MLFFMSTDITNKPREKGVFDNFTNLYEFSKTLTFGLIPLKWDDNKKMIVEDEDFSVLRKYGVIEEDKRIAESIKIAKFYLNILHRELIGKVLGSLKFEKKNLENYDRLLGEIEKNNKNENISEDKKKEIRKNFKKELSIAQDILLKKVGEVFESNGSGILSSKNCLDELTKRFTRQEVDKLRRENKDIGVEYPDVAYREKDGKEETKSFFAMDVGYLDDFHKNRKQLYSVKGKKNSLGRRILDNFEIFCKNKKLYEKYKNLDIDFSEIERNFNLTLEKVFDFDNYNERLTQEGLDEYAKILGGESNKQERTANIHGLNQIINLYIQKKQSEQKAEQKETGKKKIKFNKKDYPTFTCLQKQILSQVFRKEIIIESDRDLIRELKFFVEESKEKVDKARGIIEFLLNHEENDIDLAMVYLPKSKINSFVYKVFKEPQDFLSVFQDGASNLDFVSFDKIKTHLENNKLTYKIFFKTLIKENHDFESFLILLQQEIDLLIDGGETVTLGGKKESITSLDEKKNRLKEKLGWFEGKVRENEKMKDEEEGEFCSTVLAYSQAVLNITKRAEIFWLNEKQDAKVGEDNKDMIFYKKFDEFADDGFAPFFYFDKFGNYLKRRSRNTTKEIKLHFGNDDLLEGWDMNKEPEYWSFILRDRNQYYLGIGKKDGEIFHKKLGNSVEAVKEAYELENEADFYEKIDYKQLNIDRFEGIAFPKKTKTEEAFRQVCKKRADEFLGGDTYEFKILLAIKKEYDDFKARRQKEKDWDSKFSKEKMSKLIEYYITCLGKRDDWKRFNLNFRQPKEYEDRSDFVRHIQRQAYWIDPRKVSKDYVDKKVAEGEMFLFKVHNKDFYDFERKSEDKKNHTANLFTQYLLELFSCENIKNIKSKDLIESIFELDGKAEIRFRPKTDDVKLKIYQKKGKDVTYADKRDGNKEKEVIQHRRFAKDALTLHLKIRLNFGKHVNLFDFNKLVNTELFAKVPVKILGMDRGENNLIYYCFLDEHGEIENGKCGSLNRVGEQIITLEDDKKVKEPVDYFQLLVDREGQRDWEQKNWQKMTRIKDLKKAYLGNVVSWISKEMLSGIKEGVVTIGVLEDLNSNFKRTRFFRERQVYQGFEKALVNKLGYLVDKKYDNYRNVYQFAPIVDSVEEMEKNKQIGTLVYVPASYTSKICPHPKCGWRERLYMKNSASKEKIVGLLKSDGIKISYDQKNDRFYFEYQWEQEHKSDGKKKKYSGVDKVFSNVSRMRWDVEQKKSIDFVDGTDGSITNKLKSLLKGKGIELDNINQQIVNQQKELGVEFFQSIIFYFNLIMQIRNYDKEKSGSEADYIQCPSCLFDSRKPEMNGKLSAITNGDANGAYNIARKGFMQLCRIRENPQEPMKLITNREWDEAVREWDIYSAAQKIPVLSEEN

SEQ ID NO: 118 - protein, 1373 aa, synthetic construct
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 119 - protein, 1352 aa, synthetic construct
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 120 - protein, 1334 aa, synthetic construct
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 121 - protein, 1331 aa, synthetic construct
MKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 122 - protein, 1326 aa, synthetic construct
MKVMENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 123 - protein, 1318 aa, synthetic construct
MTNKFTNQYSLSKTLRFELIPQGKTLEFIQEKGLLSQDKQRAESYQEMKKTIDKFHKYFIDLALSNAKLTHLETYLELYNKSAETKKEQKFKDDLKKVQDNLRKEIVKSFSDGDAKSIFAILDKKELITVELEKWFENNEQKDIYFDEKFKTFTTYFTGFHQNRKNMYSVEPNSTAIAYRLIHENLPKFLENAKAFEKIKQVESLQVNFRELMGEFGDEGLIFVNELEEMFQINYYNDVLSQNGITIYNSIISGFTKNDIKYKGLNEYINNYNQTKDKKDRLPKLKQLYKQILSDRISLSFLPDAFTDGKQVLKAIFDFYKINLLSYTIEGQEESQNLLLLIRQTIENLSSFDTQKIYLKNDTHLTTISQQVFGDFSVFSTALNYWYETKVNPKFETEYSKANEKKREILDKAKAVFTKQDYFSIAFLQEVLSEYILTLDHTSDIVKKHSSNCIADYFKNHFVAKKENETDKTFDFIANITAKYQCIQGILENADQYEDELKQDQKLIDNLKFFLDAILELLHFIKPLHLKSESITEKDTAFYDVFENYYEALSLLTPLYNMVRNYVTQKPYSTEKIKLNFENAQLLNGWDANKEGDYLTTILKKDGNYFLAIMDKKHNKAFQKFPEGKENYEKMVYKLLPGVNKMLPKVFFSNKNIAYFNPSKELLENYKKETHKKGDTFNLEHCHTLIDFFKDSLNKHEDWKYFDFQFSETKSYQDLSGFYREVEHQGYKINFKNIDSEYIDGLVNEGKLFLFQIYSKDFSPFSKGKPNMHTLYWKALFEEQNLQNVIYKLNGQAEIFFRKASIKPKNIILHKKKIKIAKKHFIDKKTKTSEIVPVQTIKNLNMYYQGKISEKELTQDDLRYIDNFSIFNEKNKTIDIIKDKRFTVDKFQFHVPITMNFKATGGSYINQTVLEYLQNNPEVKIIGLDRGERHLVYLTLIDQQGNILKQESLNTITDSKISTPYHKLLDNKENERDLARKNWGTVENIKELKEGYISQVVHKIATLMLEENAIVVMEDLNFGFKRGRFKVEKQIYQKLEKMLIDKLNYLVLKDKQPQELGGLYNALQLTNKFESFQKMGKQSGFLFYVPAWNTSKIDPTTGFVNYFYTKYENVDKAKAFFEKFEAIRFNAEKKYFEFEVKKYSDFNPKAEGTQQAWTICTYGERIETKRQKDQNNKFVSTPINLTEKIEDFLGKNQIVYGDGNCIKSQIASKDDKAFFETLLYWFKMTLQMRNSETRTDIDYLISPVMNDNGTFYNSRDYEKLENPTLPKDADANGAYHIAKKGLMLLNKIDQADLTKKVDLSISNRDWLQFVQKNK

SEQ ID NO: 124 - protein, 1310 aa, synthetic construct
MFEKLSNIVSISKTIRFKLIPVGKTLENIEKLGKLEKDFERSDFYPILKNISDDYYRQYIKEKLSDLNLDWQKLYDAHELLDSSKKESQKNLEMIQAQYRKVLFNILSGELDKSGEKNSKDLIKNNKALYGKLFKKQFILEVLPDFVNNNDSYSEEDLEGLNLYSKFTTRLKNFWETRKNVFTDKDIVTAIPFRAVNENFGFYYDNIKIFNKNIEYLENKIPNLENELKEADILDDNRSVKDYFTPNGFNYVITQDGIDVYQAIRGGFTKENGEKVQGINEILNLTQQQLRRKPETKNVKLGVLTKLRKQILEYSESTSFLIDQIEDDNDLVDRINKFNVSFFESTEVSPSLFEQIERLYNALKSIKKEEVYIDARNTQKFSQMLFGQWDVIRRGYTVKITEGSKEEKKKYKEYLELDETSKAKRYLNIREIEELVNLVEGFEEVDVFSVLLEKFKMNNIERSEFEAPIYGSPIKLEAIKEYLEKHLEEYHKWKLLLIGNDDLDTDETFYPLLNEVISDYYIIPLYNLTRNYLTRKHSDKDKIKVNFDFPTLADGWSESKISDNRSIILRKGGYYYLGILIDNKLLINKKNKSKKIYEILIYNQIPEFSKSIPNYPFTKKVKEHFKNNVSDFQLIDGYVSPLIITKEIYDIKKEKKYKKDFYKDNNTNKNYLYTIYKWIEFCKQFLYKYKGPNKESYKEMYDFSTLKDTSLYVNLNDFYADVNSCAYRVLFNKIDENTIDNAVEDGKLLLFQIYNKDFSPESKGKKNLHTLYWLSMFSEENLRTRKLKLNGQAEIFYRKKLEKKPIIHKEGSILLNKIDKEGNTIPENIYHECYRYLNKKIGREDLSDEAIALFNKDVLKYKEARFDIIKDRRYSESQFFFHVPITFNWDIKTNKNVNQIVQGMIKDGEIKHIIGIDRGERHLLYYSVIDLEGNIVEQGSLNTLEQNRFDNSTVKVDYQNKLRTREEDRDRARKNWTNINKIKELKDGYLSHVVHKLSRLIIKYEAIVIMENLNQGFKRGRFKVERQVYQKFELALMNKLSALSFKEKYDERKNLEPSGILNPIQACYPVDAYQELQGQNGIVFYLPAAYTSVIDPVTGFTNLFRLKSINSSKYEEFIKKFKNIYFDNEEEDFKFIFNYKDFAKANLVILNNIKSKDWKISTRGERISYNSKKKEYFYVQPTEFLINKLKELNIDYENIDIIPLIDNLEEKAKRKILKALFDTFKYSVQLRNYDFENDYIISPTADDNGNYYNSNEIDIDKTNLPNNGDANGAFNIARKGLLLKDRIVNSNESKVDLKIKNEDWINFIIS

SEQ ID NO: 125 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 126 - protein, 1307 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILSNFAAIPMIFDEIAQNKDNLAQISIKYQNQGKKDLLQASAEEDVKAIKDLLDQTNNLLHRLKIFHISQSEDKANILDKDEHFYLVFEECYFELANIVPLYNKIRNYITQKPYSDEKFKLNFENSTLASGWDKNKESANTAILFIKDDKYYLGIMDKKHNKIFSDKAIEENKGEGYKKIVYKQIADASKDIQNLMIIDGKTVCKKGRKDRNGVNRQLLSLKRKHLPENIYRIKETKSYLKNEARFSRKDLYDFIDYYKDRLDYYDFEFELKPSNEYSDFNDFTNHIGSQGYKLTFENISQDYINSLVNEGKLYLFQIYSKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDNFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 127 - protein, 1305 aa, Eubacterium sp.
MNKAADNYTGGNYDEFIALSKVQKTLRNELKPTPFTAEHIKQRGIISEDEYRAQQSLELKKIADEYYRNYITHKLNDINNLDFYNLFDAIEEKYKKNDKDNRDKLDLVEKSKRGEIAKMLSADDNFKSMFEAKLITKLLPDYVERNYTGEDKEKALETLALFKGFTTYFKGYFKTRKNMFSGEGGASSICHRIVNVNASIFYDNLKTFMRIQEKAGDEIALIEEELTEKLDGWRLEHIFSRDYYNEVLAQKGIDYYNQICGDINKHMNLYCQQNKFKANIFKMMKIQKQIMGISEKAFEIPPMYQNDEEVYASFNEFISRLEEVKLTDRLINILQNINIYNTAKIYINARYYTNVSSYVYGGWGVIDSAIERYLYNTIAGKGQSKVKKIENAKKDNKFMSVKELDSIVAEYEPDYFNAPYIDDDDNAVKAFGGQGVLGYFNKMSELLADVSLYTIDYNSDDSLIENKESALRIKKQLDDIMSLYHWLQTFIIDEVVEKDNAFYAELEDICCELENVVTLYDRIRNYVTKKPYSTQKFKLNFASPTLAAGWSRSKEFDNNAIILLRNNKYYIAIFNVNNKPDKQIIKGSEEQRLSTDYKKMVYNLLPGPNKMLPKVFIKSDTGKRDYNPSSYILEGYEKNRHIKSSGNFDINYCHDLIDYYKACINKHPEWKNYGFKFKETNQYNDIGQFYKDVEKQGYSISWAYISEEDINKLDEEGKIYLFEIYNKDLSAHSTGRDNLHTMYLKNIFSEDNLKNICIELNGEAELFYRKSSMKSNITHKKDTILVNKTYINETGVRVSLSDEDYMKVYNYYNNNYVIDTENDKNLIDIIEKIGHRKSKIDIVKDKRYTEDKYFLYLPITINYGIEDENVNSKIIEYIAKQDNMNVIGIDRGERNLIYISVIDNKGNIIEQKSFNLVNNYDYKNKLKNMEKTRDNARKNWQEIGKIKDVKSGYLSGVISKIARMVIDYNAIIVMEDLNKGFKRGRFKVERQVYQKFENMLISKLNYLVFKERKADENGGILRGYQLTYIPKSIKNVGKQCGCIFYVPAAYTSKIDPATGFINIFDFKKYSGSGINAKVKDKKEFLMSMNSIRYINECSEEYEKIGHRELFAFSFDYNNFKTYNVSSPVNEWTAYTYGERIKKLYKDGRWLRSEVLNLTENLIKLMEQYNIEYKDGHDIREDISHMDETRNADFICSLFEELKYTVQLRNSKSEAEDENYDRLVSPILNSSNGFYDSSDYMENENNTTHTMPKDADANGAYCIALKGLYEINKIKQNWSDDKKFKENELYINVTEWLDYIQNRRFE

SEQ ID NO: 128 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYEKFEFNIEDCRKFIDFYKESISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKKSIPKKITHPAKEAIANKNKDNPKKESFFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSILNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 129 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSDDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILANFAAIPMIFDEIAQNKDNLAQISLKYQNQGKKDLLQASAEEDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYEKFEFNIEDCRKFIDFYKESISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 130 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 131 - protein, 1285 aa, unidentified
MKSFDSFTNLYSLSKTLKFEMRPVGNTQKMLDNAGVFEKDKLIQKKYGKTKPYFDRLHREFIEEALTGVELIGLDENFRTLVDWQKDKKNNVAMKAYENSLQRLRTEIGKIFNLKAEDWVKNKYPILGLKNKNTDILFEEAVFGILKARYGEEKDTFIEVEEIDKTGKSKINQISIFDSWKGFTGYFKKFFETRKNFYKNDGTSTAIATRIIDQNLKRFIDNLSIVESVRQKVDLAETEKSFSISLSQFFSIDFYNKCLLQDGIDYYNKIIGGETLKNGEKLIGLNELINQYRQNNKDQKIPFFKLLDKQILSEKILFLDEIKNDTELIEALSQFAKTAEEKTKIVKKLFADFVENNSKYDLAQIYISQEAFNTISNKWTSETETFAKYLFEAMKSGKLAKYEKKDNSYKFPDFIALSQMKSALLSISLEGHFWKEKYYKISKFQEKTNWEQFLAIFLYEFNSLFSDKINTKDGETKQVGYYLFAKDLHNLILSEQIDIPKDSKVTIKDFADSVLTIYQMAKYFAVEKKRAWLAEYELDSFYTQPDTGYLQFYDNAYEDIVQVYNKLRNYLTKKPYSEEKWKLNFENSTLANGWDKNKESDNSAVILQKGGKYYLGLITKGHNKIFDDRFQEKFIVGIEGGKYEKIVYKFFPDQAKMFPKVCFSAKGLEFFRPSEEILRIYNNAEFKKGETYSIDSMQKLIDFYKDCLTKYEGWACYTFRHLKPTEEYQNNIGEFFRDVAEDGYRIDFQGISDQYIHEKNEKGELHLFEIHNKDWNLDKARDGKSKTTQKNLHTLYFESLFSNDNVVQNFPIKLNGQAEIFYRPKTEKDKLESKKDKKGNKVIDHKRYSENKIFFHVPLTLNRTKNDSYRFNAQINNFLANNKDINIIGVDRGEKHLVYYSVITQASDILESGSLNELNGVNYAEKLGKKAENREQARRDWQDVQGIKDLKKGYISQVVRKLADLAIKHNAIIILEDLNMRFKQVRGGIEKSIYQQLEKALIDKLSFLVDKGEKNPEQAGHLLKAYQLSAPFETFQKMGKQTGIIFYTQASYTSKSDPVTGWRPHLYLKYFSAKKAKDDIAKFTKIEFVNDRFELTYDIKDFQQAKEYPNKTVWKVCSNVERFRWDKNLNQNKGGYTHYTNITENIQELFTKYGIDITKDLLTQISTIDEKQNTSFFRDFIFYFNLICQIRNTDDSEIAKKNGKDDFILSPVEPFFDSRKDNGNKLPENGDDNGAYNIARKGIVILNKISQYSEKNENCEKMKWGDLYVSNIDWDNFVTQANARH

SEQ ID NO: 132 - protein, 1282 aa, Eubacterium eligens
MNGNRSIVYREFVGVTPVAKTLRNELRPVGHTQEHIIQNGLIQEDELRQEKSTELKNIMDDYYREYIDKSLSGLTDLDFTLLFELMNSVQSSLSKDNKKALEKEHNKMREQICTHLQSDSDYKNMFNAKLFKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEIKKRLDMYMNMYHWVKAFIVDEVLDRDEMFYSDIDDIYNILENIVPLYNRVRNYVTQKPYTSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKNIVIKLNGQAELFYRKASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDVRIDMEKMYEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 133 - protein, 508 aa, Eubacterium ramulus
MIKKTIDTVLNVRPIFVGIQHLYFYEGPCRFGEGDELMPEYDAMMNQEMNAAYVNEVVQHETEGVHIMDPIYVERDDWFRSPEAMYEKMAEDIDKVDFYLFHFGIGRGDIYLEFAERYKKPVGAAPGLCCDGIGNTAAVKNRGLEAYAFMSWDEFDTWMRVLRVRKCLKNTRVLLAVRWDSNRSYSSYDNFINQSDVTNKWGIQFRHVNVHELLDQTHPVDPTTNPSTPGRKALNINDEDMKEIEKITDELIANAEACTMEPDMVKKTIQAYYTVQKLLDAYDCNAFTAPCPDLCSTRRFSEEKFTLCMTHSLNDENGISSACEYDINSVIGKVIMTNLSGKAPYMGNTNAIVFDKEGHMIPFHKFNDNTIEDIADKTNLYMTFHSTPNRNLKGLKAEKERYRLAPFAYSGFGATIRYDFAQDIGQVITMIRISPDATKIFIAKGTISGGAGYEMKNCDQGVFFNVADKVDFYHKQQYFGNHTVLAYGDYVEELKMLAEALGIEAVIA

SEQ ID NO: 134 - protein, 1273 aa, Flavobacterium sp.
MKNFSNLYQVSKTVRFELKPIGNTLENIKNKSLLKNDSIRAESYQKMKKTIDEFHKYFIDLALNNKKLSYLNEYIALYTQSAEAKKEDKFKADFKKVQDNLRKEIVSSFTEGEAKAIFSVLDKKELITIELEKWKNENNLAVYLDESFKSFTTYFTGFHQNRKNMYSAEANSTAIAYRLIHENLPKFIENSKAFEKSSQIAELQPKIEKLYKEFEAYLNVNSISELFEIDYFNEVLTQKGITVYNNIIGGRTATEGKQKIQGLNEIINLYNQTKPKNERLPKLKQLYKQILSDRISLSFLPDAFTEGKQVLKAVFEFYKINLLSYKQDGVEESQNLLELIQQVVKNLGNQDVNKIYLKNDTSLTTIAQQLFGDFSVFSAALQYRYETVVNPKYTAEYQKANEAKQEKLDKEKIKFVKQDYFSIAFLQEVVADYVKTLDENLDWKQKYTPSCIADYFTTHFIAKKENEADKTFNFIANIKAKYQCIQGILEQADDYEDELKQDQKLIDNIKFFLDAILEVVHFIKPLHLKSESITEKDNAFYDVFENYYEALNVVTPLYNMVRNYVTQKPYSTEKIKLNFENAQLLNGWDANKEKDYLTTILKRDGNYFLAIMDKKHNKTFQQFTEDDENYEKIVYKLLPGVNKMLPKVFFSNKNIAFFNPSKEILDNYKNNTHKKGATFNLKDCHALIDFFKDSLNKHEDWKYFDFQFSETKTYQDLSGFYKEVEHQGYKINFKKVSVSQIDTLIEEGKMYLFQIYNKDFSPYAKGKPNMHTLYWKALFETQNLENVIYKLNGQAEIFFRKASIKKKNIITHKAHQPIAAKNPLTPTAKNTFAYDLIKDKRYTVDKFQFHVPITMNFKATGNSYINQDVLAYLKDNPEVNIIGLDRGERHLVYLTLIDQKGTILLQESLNVIQDEKTHTPYHTLLDNKEIARDKARKNWGSIESIKELKEGYISQVVHKITKMMIEHNAIVVMEDLNFGFKRGRFKVEKQIYQKLEKMLIDKLNYLVLKDKQPHELGGLYNALQLTNKFESFQKMGKQSGFLFYVPAWNTSKIDPTTGFVNYFYTKYENVEKAKTFFSKFDSILYNKTKGYFEFVVKNYSDFNPKAADTRQEWTICTHGERIETKRQKEQNNNFVSTTIQLTEQFVNFFEKVGLDLSKELKTQLIAQNEKSFFEELFHLLKLTLQMRNSESHTEIDYLISPVANEKGIFYDSRKATASLPIDADANGAYHIAKKGLWIMEQINKTNSEDDLKKVKLAISNREWLQYVQQVQKK

SEQ ID NO: 135 - protein, 1264 aa, Prevotella brevis
MKQFTNLYQLSKTLRFELKPIGKTLEHINANGFIDNDAHRAESYKKVKKLIDDYHKDYIENVLNNFKLNGEYLQAYFDLYSQDTKDKQFKDIQDKLRKSIASALKGDDRYKTIDKKELIRQDMKTFLKKDTDKALLDEFYEFTTYFTGYHENRKNMYSDEAKSTAIAYRLIHDNLPKFIDNIAVFKKIANTSVADNFSTIYKNFEEYLNVNSIDEIFSLDYYNIVLTQTQIEVYNSIIGGRTLEDDTKIQGINEFVNLYNQQLANKKDRLPKLKPLFKQILSDRVQLSWLQEEFNTGADVLNAVKEYCTSYFDNVEESVKVLLTGISDYDLSKIYITNDLALTDVSQRMFGEWSIIPNAIEQRLRSDNPKKTNEKEEKYSDRISKLKKLPKSYSLGYINECISELNGIDIADYYATLGAINTESKQEPSIPTSIQVHYNALKPILDTDYPREKNLSQDKLTVMQLKDLLDDFKALQHFIKPLLGNGDEAEKDEKFYGELMQLWEVIDSITPLYNKVRNYCTRKPFSTEKIKVNFENAQLLDGWDENKESTNASIILRKNGMYYLGIMKKEYRNILTKPMPSDGDCYDKVVYKFFKDITTMVPKCTTQMKSVKEHFSNSNDDYTLFEKDKFIAPVVITKEIFDLNNVLYNGVKKFQIGYLNNTGDSFGYNHAVEIWKSFCLKFLKAYKSTSIYDFSSIEKNIGCYNDLNSFYGAVNLLLYNLTYRKVSVDYIHQLVDEDKMYLFMIYNKDFSTYSKGTPNMHTLYWKMLFDESNLNDVVYKLNGQAEVFYRKKSITYQHPTHPANKPIDNKNVNNPKKQSNFEYDLIKDKRYTVDKFMFHVPITLNFKGMGNGDINMQVREYIKTTDDLHFIGIDRGERHLLYICVINGKGEIVEQYSLNEIVNNYKGTEYKTDYHTLLSERDKKRKEERSSWQTIEGIKELKSGYLSQVIHKITQLMIKYNAIVLLEDLNMGFKRGRQKVESSVYQQFEKALIDKLNYLVDKNKDANEIGGLLHAYQLTNDPKLPNKNSKQSGFLFYVPAWNTSKIDPVTGFVNLLDTRYENVAKAQAFFKKFDSIRYNKEYDRFEFKFDYSNFTAKAEDTRTQWTLCTYGTRIETFRNAEKNSNWDSREIDLTTEWKTLFTQHNIPLNANLKEAILLQANKNFYTDILHLMKLTLQMRNSVTGTDIDYMVSPVANECGEFFDSRKVKEGLPVNADANGAYNIARKGLWLAQQIKNANDLSDVKLAITNKEWLQFAQKKQYLKD

SEQ ID NO: 136 - protein, 1264 aa, Moraxella caprae
MLFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNR

SEQ ID NO: 137 - protein, 1263 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 138 - protein, 1262 aa, Porphyromonas crevioricanis
MPWIDLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYEPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 139 - protein, 1262 aa, unidentified
MRKFNEFVGLYPISKTLRFELKPIGKTLEHIQRNKLLEHDAVRADDYVKVKKIIDKYHKCLIDEALSGFTFDTEADGRSNNSLSEYYLYYNLKKRNEQEQKTFKTIQNNLRKQIVNKLTQSEKYKRIDKKELITTDLPDFLTNESEKELVEKFKNFTTYFTEFHKNRKNMYSKEEKSTAIAFRLINENLPKFVDNIAAFEKVVSSPLAEKINALYEDFKEYLNVEEISRVFRLDYYDELLTQKQIDLYNAIVGGRTEEDNKIQIKGLNQYINEYNQQQTDRSNRLPKLKPLYKQILSDRESVSWLPPKFDSDKNLLIKIKECYDALSEKEKVFDKLESILKSLSTYDLSKIYISNDSQLSYISQKMFGRWDIISKAIREDCAKRNPQKSRESLEKFAERIDKKLKTIDSISIGDVDECLAQLGETYVKRVEDYFVAMGESEIDDEQTDTTSFKKNIEGAYESVKELLNNADNITDNNLMQDKGNVEKIKTLLDAIKDLQRFIKPLLGKGDEADKDGVFYGEFTSLWTKLDQVTPLYNMVRNYLTSKPYSTKKIKLNFENSTLMDGWDLNKEPDNTTVIFCKDGLYYLGIMGKKYNRVFVDREDLPHDGECYDKMEYKLLPGANKMLPKVFFSETGIQRFLPSEELLGKYERGTHKKGAGFDLGDCRALIDFFKKSIERHDDWKKFDFKFSDTSTYQDISEFYREVEQQGYKMSFRKVSVDYIKSLVEEGKLYLFQIYNKDFSAHSKGTPNMHTLYWKMLFDEENLKDVVYKLNGEAEVFFRKSSITVQSPTHPANSPIKNKNKDNQKKESKFEYDLIKDRRYTVDKFLFHVPITMNFKSVGGSNINQLVKRHIRSATDLHIIGIDRGERHLLYLTVIDSRGNIKEQFSLNEIVNEYNGNTYRTDYHELLDTREGERTEARRNWQTIQNIRELKEGYLSQVIHKISELAIKYNAVIVLEDLNFGFMRSRQKVEKQVYQKFEKMLIDKLNYLVDKKKPVAETGGLLRAYQLTGEFESFKTLGKQSGILFYVPAWNTSKIDPVTGFVNLFDTHYENIEKAKVFFDKFKSIRYNSDKDWFEFVVDDYTRFSPKAEGTRRDWTICTQGKRIQICRNHQRNNEWEGQEIDLTKAFKEHFEAYGVDISKDLREQINTQNKKEFFEELLRLLRLTLQMRNSMPSSDIDYLISPVANDTGCFFDSRKQAELKENAVLPMNADANGAYNIARKGLLAIRKMKQEENDSAKISLAISNKEWLKFAQTKPYLED

SEQ ID NO: 140 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 141 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRHYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 142 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 143 - protein, 1259 aa, Synergistes jonesii
MANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 144 - protein, 1262 aa, Synergistes jonesii
MENMANSLKDFTNIYQLSKTLRFELKPIGKTEEHINRKLIIMHDEKRGEDYKSVTKLIDDYHRKFIHETLDPAHFDWNPLAEALIQSGSKNNKALPAEQKEMREKIISMFTSQAVYKKLFKKELFSELLPEMIKSELVSDLEKQAQLDAVKSFDKFSTYFTGFHENRKNIYSKKDTSTSIAFRIVHQNFPKFLANVRAYTLIKERAPEVIDKAQKELSGILGGKTLDDIFSIESFNNVLTQDKIDYYNQIIGGVSGKAGDKKLRGVNEFSNLYRQQHPEVASLRIKMVPLYKQILSDRTTLSFVPEALKDDEQAINAVDGLRSELERNDIFNRIKRLFGKNNLYSLDKIWIKNSSISAFSNELFKNWSFIEDALKEFKENEFNGARSAGKKAEKWLKSKYFSFADIDAAVKSYSEQVSADISSAPSASYFAKFTNLIETAAENGRKFSYFAAESKAFRGDDGKTEIIKAYLDSLNDILHCLKPFETEDISDIDTEFYSAFAEIYDSVKDVIPVYNAVRNYTTQKPFSTEKFKLNFENPALAKGWDKNKEQNNTAIILMKDGKYYLGVIDKNNKLRADDLADDGSAYGYMKMNYKFIPTPHMELPKVFLPKRAPKRYNPSREILLIKENKTFIKDKNFNRTDCHKLIDFFKDSINKHKDWRTFGFDFSDTDSYEDISDFYMEVQDQGYKLTFTRLSAEKIDKWVEEGRLFLFQIYNKDFADGAQGSPNLHTLYWKAIFSEENLKDVVLKLNGEAELFFRRKSIDKPAVHAKGSMKVNRRDIDGNPIDEGTYVEICGYANGKRDMASLNAGARGLIESGLVRITEVKHELVKDKRYTIDKYFFHVPFTINFKAQGQGNINSDVNLFLRNNKDVNIIGIDRGERNLVYVSLIDRDGHIKLQKDFNIIGGMDYHAKLNQKEKERDTARKSWKTIGTIKELKEGYLSQVVHEIVRLAVDNNAVIVMEDLNIGFKRGRFKVEKQVYQKFEKMLIDKLNYLVFKDAGYDAPCGILKGLQLTEKFESFTKLGKQCGIIFYIPAGYTSKIDPTTGFVNLFNINDVSSKEKQKDFIGKLDSIRFDAKRDMFTFEFDYDKFRTYQTSYRKKWAVWTNGKRIVREKDKDGKFRMNDRLLTEDMKNILNKYALAYKAGEDILPDVISRDKSLASEIFYVFKNTLQMRNSKRDTGEDFIISPVLNAKGRFFDSRKTDAALPIDADANGAYHIALKGSLVLDAIDEKLKEDGRIDYKDMAVSNPKWFEFMQTRKFDF

SEQ ID NO: 145 - protein, 1257 aa, Prevotella bryantii
MQINNLKIIYMKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 146 - protein, 1253 aa, Prevotella albensis
MNIKNFTGLYPLSKTLRFELKPIGKTKENIEKNGILTKDEQRAKDYLIVKGFIDEYHKQFIKDRLWDFKLPLESEGEKNSLEEYQELYELTKRNDAQEADFTEIKDNLRSSITEQLTKSGSAYDRIFKKEFIREDLVNFLEDEKDKNIVKQFEDFTTYFTGFYENRKNMYSSEEKSTAIAYRLIHQNLPKFMDNMRSFAKIANSSVSEHFSDIYESWKEYLNVNSIEEIFQLDYFSETLTQPHIEVYNYIIGKKVLEDGTEIKGINEYVNLYNQQQKDKSKRLPFLVPLYKQILSDREKLSWIAEEFDSDKKMLSAITESYNHLHNVLMGNENESLRNLLLNIKDYNLEKINITNDLSLTEISQNLFGRYDVFTNGIKNKLRVLTPRKKKETDENFEDRINKIFKTQKSFSIAFLNKLPQPEMEDGKPRNIEDYFITQGAINTKSIQKEDIFAQIENAYEDAQVFLQIKDTDNKLSQNKTAVEKIKTLLDALKELQHFIKPLLGSGEENEKDELFYGSFLAIWDELDTITPLYNKVRNWLTRKPYSTEKIKLNFDNAQLLGGWDVNKEHDCAGILLRKNDSYYLGIINKKTNHIFDTDITPSDGECYDKIDYKLLPGANKMLPKVFFSKSRIKEFEPSEAIINCYKKGTHKKGKNFNLTDCHRLINFFKTSIEKHEDWSKFGFKFSDTETYEDISGFYREVEQQGYRLTSHPVSASYIHSLVKEGKLYLFQIWNKDFSQFSKGTPNLHTLYWKMLFDKRNLSDVVYKLNGQAEVFYRKSSIEHQNRIIHPAQHPITNKNELNKKHTSTFKYDIIKDRRYTVDKFQFHVPITINFKATGQNNINPIVQEVIRQNGITHIIGIDRGERHLLYLSLIDLKGNIIKQMTLNEIINEYKGVTYKTNYHNLLEKREKERTEARHSWSSIESIKELKDGYMSQVIHKITDMMVKYNAIVVLEDLNGGFMRGRQKVEKQVYQKFEKKLIDKLNYLVDKKLDANEVGGVLNAYQLTNKFESFKKIGKQSGFLFYIPAWNTSKIDPITGFVNLFNTRYESIKETKVFWSKFDIIRYNKEKNWFEFVFDYNTFTTKAEGTRTKWTLCTHGTRIQTFRNPEKNAQWDNKEINLTESFKALFEKYKIDITSNLKESIMQETEKKFFQELHNLLHLTLQMRNSVTGTDIDYLISPVADEDGNFYDSRINGKNFPENADANGAYNIARKGLMLIRQIKQADPQKKFKFETITNKDWLKFAQDKPYLKD

SEQ ID NO: 147 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYAWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 148 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEEYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKIYFRSGTSLTDVSRKVFGEWSIINRALDNYYATTYPIKPREKSEKYEERKEKWLKQDFNVSLIQTAIDEYDNETVKGKNSGKVIVDYFAKFCDDKETDLIQKVNEGYIAVKDLLNTPYPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKENLYYLGIMDKRHNRIFRNVPKADKKDSCYEKMVYKLLPGANKMLPKVFFSQSRIQEFTPSAKLLENYENETHKKGDNFNLNHCHQLIDFFKDSINKHEDWKNFDFRFSATSTYADLSGFYHEVEHQGYKISFQSIADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHVPITMNFKAEGIFNMNQRVNQFLKANPDINIIGIDRGERHLLYYTLINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLKQAKDFFEKFDSIRLNSKADYFEFAFDFKNFTGKADGGRTKWTVCTTNEDRYAWNRALNNNRGSQEKYDITAELKSLFDGKVDYKSGKDLKQQIASQELADFFRTLMKYLSVTLSLRHNNGEKGETEQDYILSPVADSMGKFFDSRKAGDDMPKNADANGAYHIALKGLWCLEQISKTDDLKKVKLAISNKEWLEFMQTLKG

SEQ ID NO: 149 - protein, 1247 aa, Prevotella bryantii
MKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 150 - protein, 1247 aa, unidentified
MFKGDAFTGLYEVQKTLRFELVPIGLTQSYLENDWVIQKDKEVEENYGKIKAYFDLIHKEFVRQSLENAWLCQLDDFYEKYIELHNSLETRKDKNLAKQFEKVMKSLKKEFVSFFDAKWNEWKQKFSFLKKWWIDVLNEKEVLDLMAEFYPDEKELFDKFDKFFTYFSNFKESRKNFYADDGRAWAIATRAIDENLITFIKNIEDFKKLNSSFREFVNDNFSEEDKQIFEIDFYNNCLLQPWIDKYNKIVWWYSLENWEKVQWLNEKINNFKQNQNKSNSKDLKFPRMKLLYKQILGDKEKKVYIDEIRDDKNLIDLIDNSKRRNQIKIDNANDIINDFINNNAKFELDKIYLTRQSINTISSKYFSSWDYIRWYFWTGELQEFVSFYDLKETFWKIEYETLENIFKDCYVKGINTESQNNIVFETQGIYENFLNIFKFEFNQNISQISLLEWELDKIQNEDIKKNEKQVEVIKNYFDSVMSVYKMTKYFSLEKWKKRVELDTDNNFYNDFNEYLEGFEIWKDYNLVRNYITKKQVNTDKIKLNFDNSQFLTWWDKDKENERLGIILRREWKYYLWILKKWNTLNFGDYLQKEWEIFYEKMNYKQLNNVYRQLPRLLFPLTKKLNELKWDELKKYLSKYIQNFWYNEEIAQIKIEFDIFQESKEKWEKFDIDKLRKLIEYYKKWVLALYSDLYDLEFIKYKNYDDLSIFYSDVEKKMYNLNFTKIDKSLIDGKVKSWELYLFQIYNKDFSESKKEWSTENIHTKYFKLLFNEKNLQNLVVKLSWWADIFFRDKTENLKFKKDKNGQEILDHRRFSQDKIMFHISITLNANCWDKYWFNQYVNEYMNKERDIKIIWIDRWEKHLAYYCVIDKSWKIFNNEIWTLNELNWVNYLEKLEKIESSRKDSRISWWEIENIKELKNGYISQVINKLTELIVKYNAIIVFEDLNIWFKRWRQKIEKQIYQKLELALAKKLNYLTQKDKKDDEILWNLKALQLVPKVNDYQDIWNYKQSWIMFYVRANYTSVTCPNCWLRKNLYISNSATKENQKKSLNSIAIKYNDWKFSFSYEIDDKSWKQKQSLNKKKFIVYSDIERFVYSPLEKLTKVIDVNKKLLELFRDFNLSLDINKQIQEKDLDSVFFKSLTHLFNLILQLRNSDSKDNKDYISCPSCYYHSNNWLQWFEFNWDANWAYNIARKGIILLDRIRKNQEKPDLYVSDIDWDNFVQSNQFPNTIIPIQNIEKQVPLNIKI

SEQ ID NO: 151 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 152 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEKARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 153 - protein, 1238 aa, unidentified
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 154 - protein, 1235 aa, Anaerovibrio sp.
MVAFIDEFVGQYPVSKTLRFEARPVPETKKWLESDQCSVLFNDQKRNEYYGVLKELLDDYYRAYIEDALTSFTLDKALLENAYDLYCNRDTNAFSSCCEKLRKDLVKAFGNLKDYLLGSDQLKDLVKLKAKVDAPAGKGKKKIEVDSRLINWLNNNAKYSAEDREKYIKAIESFEGFVTYLTNYKQARENMFSSEDKSTAIAFRVIDQNMVTYFGNIRIYEKIKAKYPELYSALKGFEKFFSPTAYSEILSQSKIDEYNYQCIGRPIDDADFKGVNSLINEYRQKNGIKARELPVMSMLYKQILSDRDNSFMSEVINRNEEAIECAKNGYKVSYALFNELLQLYKKIFTEDNYGNIYVKTQPLTELSQALFGDWSILRNALDNGKYDKDIINLAELEKYFSEYCKVLDADDAAKIQDKFNLKDYFIQKNALDATLPDLDKITQYKPHLDAMLQAIRKYKLFSMYNGRKKMDVPENGIDFSNEFNAIYDKLSEFSILYDRIRNFATKKPYSDEKMKLSFNMPTMLAGWDYNNETANGCFLFIKDGKYFLGVADSKSKNIFDFKKNPHLLDKYSSKDIYYKVKYKQVSGSAKMLPKVVFAGSNEKIFGHLISKRILEIREKKLYTAAAGDRKAVAEWIDFMKSAIAIHPEWNEYFKFKFKNTAEYDNANKFYEDIDKQTYSLEKVEIPTEYIDEMVSQHKLYLFQLYTKDFSDKKKKKGTDNLHTMYWHGVFSDENLKAVTEGTQPIIKLNGEAEMFMRNPSIEFQVTHEHNKPIANKNPLNTKKESVFNYDLIKDKRYTERKFYFHCPITLNFRADKPIKYNEKINRFVENNPDVCIIGIDRGERHLLYYTVINQTGDILEQGSLNKISGSYTNDKGEKVNKETDYHDLLDRKEKGKHVAQQAWETIENIKELKAGYLSQVVYKLTQLMLQYNAVIVLENLNVGFKRGRTKVEKQVYQKFEKAMIDKLNYLVFKDRGYEMNGSYAKGLQLTDKFESFDKIGKQTGCIYYVIPSYTSHIDPKTGFVNLLNAKLRYENITKAQDTIRKFDSISYNAKADYFEFAFDYRSFGVDMARNEWVVCTCGDLRWEYSAKTRETKAYSVTDRLKELFKAHGIDYVGGENLVSHITEVADKHFLSTLLFYLRLVLKMRYTVSGTENENDFILSPVEYAPGKFFDSREATSTEPMNADANGAYHIALKGLMTIRGIEDGKLHNYGKGGENAAWFKFMQNQEYKNNG

SEQ ID NO: 155 - protein, 1233 aa, unidentified
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKLLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 156 - protein, 1235 aa, unidentified
MTMDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKLLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 157 - protein, 1231 aa, Butyrivibrio fibrisolvens
MYYESLTKLYPIKKTIRNELVPIGKTLENIKKNNILEADEDRKIAYIRVKAIMDDYHKRLINEALSGFALIDLDKAANLYLSRSKSADDIESFSRFQDKLRKAIAKRLREHENFGKIGNKDIIPLLQKLSENEDDYNALESFKNFYTYFESYNDVRLNLYSDKEKSSTVAYRLINENLPRFLDNIRAYDAVQKAGITSEELSSEAQDGLFLVNTFNNVLIQDGINTYNEDIGKLNVAINLYNQKNASVQGFRKVPKMKVLYKQILSDREESFIDEFESDTELLDSLESHYANLAKYFGSNKVQLLFTALRESKGVNVYVKNDIAKTSFSNVVFGSWSRIDELINGEYDDNNNRKKDEKYYDKRQKELKKNKSYTIEKIITLSTEDVDVIGKYIEKLESDIDDIRFKGKNFYEAVLCGHDRSKKLSKNKGAVEAIKGYLDSVKDFERDLKLINGSGQELEKNLVVYGEQEAVLSELSGIDSLYNMTRNYLTKKPFSTEKIKLNFNKPTFLDGWDYGNEEAYLGFFMIKEGNYFLAVMDANWNKEFRNIPSVDKSDCYKKVIYKQISSPEKSIQNLMVIDGKTVKKNGRKEKEGIHSGENLILEELKNTYLPKKINDIRKRRSYLNGDTFSKKDLTEFIGYYKQRVIEYYNGYSFYFKSDDDYASFKEFQEDVGRQAYQISYVDVPVSFVDDLINSGKLYLFRVYNKDFSEYSKGRLNLHTLYFKMLFDERNLKNVVYKLNGQAEVFYRPSSIKKEELIVHRAGEEIKNKNPKRAAQKPTRRLDYDIVKDRRYSQDKFMLHTSIIMNFGAEENVSFNDIVNGVLRNEDKVNVIGIDRGERNLLYVVVIDPEGKILEQRSLNCITDSNLDIETDYHRLLDEKESDRKIARRDWTTIENIKELKAGYLSQVVHIVAELVLKYNAIICLEDLNFGFKRGRQKVEKQVYQKFEKMLIDKLNYLVMDKSREQLSPEKISGALNALQLTPDFKSFKVLGKQTGIIYYVPAYLTSKIDPMTGFANLFYVKYENVDKAKEFFSKFDSIKYNKDGKNWNTKGYFEFAFDYKKFTDRAYGRVSEWTVCTVGERIIKFKNKEKNNSYDDKVIDLTNSLKELFDSYKVTYESEVDLKDAILAIDDPAFYRDLTRRLQQTLQMRNSSCDGSRDYIISPVKNSKGEFFCSDNNDDTTPNDADANGAFNIARKGLWVLNEIRNSEEGSKINLAMSNAQWLEYAQDNTI

SEQ ID NO: 158 - protein, 1230 aa, unidentified
MHENNGKIADNFIGIYPVSKTLRFELKPVGKTQEYIEKHGILDEDLKRAGDYKSVKKIIDAYHKYFIDEALNGIQLDGLKNYYELYEKKRDNNEEKEFQKIQMSLRKQIVKRFSEHPQYKYLFKKELIKNVLPEFTKDNAEEQTLVKSFQEFTTYFEGFHQNRKNMYSDEEKSTAIAYRVVHQNLPKYIDNMRIFSMILNTDIRSDLTELFNNLKTKMDITIVEEYFAIDGFNKVVNQKGIDVYNTILGAFSTDDNTKIKGLNEYINLYNQKNKAKLPKLKPLFKQILSDRDKISFIPEQFDSDTEVLEAVDMFYNRLLQFVIENEGQITISKLLTNFSAYDLNKIYVKNDTTISAISNDLFDDWSYISKAVRENYDSENVDKNKRAAAYEEKKEKALSKIKMYSIEELNFFVKKYSCNECHIEGYFERRILEILDKMRYAYESCKILHDKGLINNISLCQDRQAISELKDFLDSIKEVQWLLKPLMIGQEQADKEEAFYTELLRIWEELEPITLLYNKVRNYVTKKPYTLEKVKLNFYKSTLLDGWDKNKEKDNLGIILLKDGQYYLGIMNRRNNKIADDAPLAKTDNVYRKMEYKLLTKVSANLPRIFLKDKYNPSEEMLEKYEKGTHLKGENFCIDDCRELIDFFKKGIKQYEDWGQFDFKFSDTESYDDISAFYKEVEHQGYKITFRDIDETYIDSLVNEGKLYLFQIYNKDFSPYSKGTKNLHTLYWEMLFSQQNLQNIVYKLNGNAEIFYRKASINQKDVVVHKADLPIKNKDPQNSKKESMFDYDIIKDKRFTCDKYQFHVPITMNFKALGENHFNRKVNRLIHDAENMHIIGIDRGERNLIYLCMIDMKGNIVKQISLNEIISYDKNKLEHKRNYHQLLKTREDENKSARQSWQTIHTIKELKEGYLSQVIHVITDLMVEYNAIVVLEDLNFGFKQGRQKFERQVYQKFEKMLIDKLNYLVDKSKGMDEDGGLLHAYQLTDEFKSFKQLGKQSGFLYYIPAWNTSKLDPTTGFVNLFYTKYESVEKSKEFINNFTSILYNQEREYFEFLFDYSAFTSKAEGSRLKWTVCSKGERVETYRNPKKNNEWDTQKIDLTFELKKLFNDYSISLLDGDLREQMGKIDKADFYKKFMKLFALIVQMRNSDEREDKLISPVLNKYGAFFETGKNERMPLDADANGAYNIARKGLWIIEKIKNTDVEQLDKVKLTISNKEWLQYAQEHIL

SEQ ID NO: 159 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 160 - protein, 1246 aa, unidentified
MLKNVGIDRLDVEKGRKNMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 161 - protein, 1227 aa, unidentified
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 162 - protein, 1224 aa, unidentified
MGLYDGFVNRYSVSKTLRFELIPQGRTREYIETNGILSDDEERAKDYKTIKRLIDEYHKDYISRCLKNVNISCLEEYYHLYNSSNRDKRHEELDALSDQMRGEIASFLTGNDEYKEQKSRDIIINERIINFASTDEELAAVKRFRKFTSYFTGFFTNRENMYSAEKKSTAIAHRIIDVNLPKYVDNIKAFNTAIEAGVFDIAEFESNFKAITDEHEVSDLLDITKYSRFIRNEDIIIYNTLLGGISMKDEKIQGLNELINLHNQKHPGKKVPLLKVLYKQILGDSQTHSFVDDQFEDDQQVINAVKAVTDTFSETLLGSLKIIINNIGHYDLDRIYIKAGQDITTLSKRALNDWHIITECLESEYDDKFPKNKKSDTYEEMRNRYVKSFKSFSIGRLNSLVTTYTEQACFLENYLGSFGGDTDKNCLTDFTNSLMEVEHLLNSEYPVTNRLITDYESVRILKRLLDSEMEVIHFLKPLLGNGNESDKDLVFYGEFEAEYEKLLPVIKVYNRVRNYLTRKPFSTEKIKLNFNSPTLLCGWSQSKEKEYMGVILRKDGQYYLGIMTPSNKKIFSEAPKPDEDCYEKMVLRYIPHPYQMLPKVFFSKSNIAFFNPSDEILRIKKQESFKKGKSFNRDDCHKFIDFYKDSINRHEEWRKFNFKFSDTDSYEDISRFYKEVENQAFSMSFTKIPTVYIDSLVDEGKLYLFKLHNKDFSEHSKGKPNLHTVYWNALFSEYNLQNTVYQLNGSAEIFFRKASIPENERVIHKKNVPITRKVAELNGKKEVSVFPYDIIKNRRYTVDKFQFHVPLKMNFKADEKKRINDDVIEAIRSNKGIHVIGIDRGERNLLYLSLINEEGRIIEQRSLNIIDSGEGHTQNYRDLLDSREKDREKARENWQEIQEIKDLKTGYLSQAIHTITKWMKEYNAIIVLEDLNDRFTNGRKKVEKQVYQKFEKMLIDKLNYYVDKDEEFDRMGGTHRALQLTEKFESFQKLGRQTGFIFYVPAWNTSKLDPTTGFVDLLYPKYKSVDATKDFIKKFDFIRFNSEKNYFEFGLHYSNFTERAIGCRDEWILCSYGNRIVNFRNAAKNNSWDYKEIDITKQLLDLFEKNGIDVKQENLIDSICEMKDKPFFKSLIANIKLILQIRNSASGTDIDYMISPAMNDRGEFFDTRKGLQQLPLDADANGAYNIAKKGLWIVDQIRNTTGNNVKMAMSNREWMHFAQESRLA

SEQ ID NO: 163 - protein, 1214 aa, unidentified
MKNVFGGFTNLYSLTKTLRFELKPTSKTQKLMKRNNVIQTDEEIDKLYHDEMKPILDEIHRRFINDALAQKIFISASLDNFLKVVKNYKVESAKKNIKQNQVKLLQKEITIKTLGLRREVVSGFITVSKKWKDKYVGLGIKLKGDGYKVLTEQAVLDILKIEFPNKAKYIDKFRGFWTYFSGFNENRKNYYSEEDKATSIANRIVNENLSRYIDNIIAFEEILQKIPNLKKFKQDLDITSYNYYLNQAGIDKYNKIIGGYIVDKDKKIQGINEKVNLYTQQTKKKLPKLKFLFKQIGSERKGFGIFEIKEGKEWEQLGDLFKLQRTKINSNGREKGLFDSLRTMYREFFDEIKRDSNSQARYSLDKIYFNKASVNTISNSWFTNWNKFAELLNIKEDKKNGEKKIPEQISIEDIKDSLSIIPKENLEELFKLTNREKHDRTRFFGSNAWVTFLNIWQNEIEESFNKLEEKEKDFKKNAAIKFQKNNLVQKNYIKEVCDRMLAIERMAKYHLPKDSNLSREEDFYWIIDNLSEQREIYKYYNAFRNYISKKPYNKSKMKLNFENGNLLGGWSDGQERNKAGVILRNGNKYYLGVLINRGIFRTDKINNEIYRTGSSKWERLILSNLKFQTLAGKGFLGKHGVSYGNMNPEKSVPSLQKFIRENYLKKYPQLTEVSNTKFLSKKDFDAAIKEALKECFTMNFINIAENKLLEAEDKGDLYLFEITNKDFSGKKSGKDNIHTIYWKYLFSESNCKSPIIGLNGGAEIFFREGQKDKLHTKLDKKGKKVFDAKRYSEDKLFFHVSITINYGKPKNIKFRDIINQLITSMNVNIIGIDRGEKHLLYYSVIDSNGIILKQGSLNKIRVGDKEVDFNKKLTERANEMKKARQSWEQIGNIKNFKEGYLSQAIHEIYQLMIKYNAIIVLEDLNTEFKAKRLSKVEKSVYKKFELKLARKLNHLILKDRNTNEIGGVLKAYQLTPTIGGGDVSKFEKAKQWGMMFYVRANYTSTTDPVTGWRKHLYISNFSNNSVIKSFFDPTNRDTGIEIFYSGKYRSWGFRYVQKETGKKWELFATKELERFKYNQTTKLCEKINLYDKFEELFKGIDKSADIYSQLCNVLDFRWKSLVYLWNLLNQIRNVDKNAEGNKNDFIQSPVYPFFDSRKTDGKTEPINGDANGALNIARKGLMLVERIKNNPEKYEQLIRDTEWDAWIQNFNKVN

SEQ ID NO: 164 - protein, 1206 aa, unidentified
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 165 - protein, 1206 aa, Butyrivibrio sp.
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 166 - protein, 1206 aa, unidentified
MENYYDSLTRQYPVTKTIRQELKPVGKTLENIKNAEIIEADKQKKEAYVKVKELMDEFHKSIIEKSLVGIKLDGLSEFEKLYKIKTKTDEDKNRISELFYYMRKQIADALKNSRDYGYVDNKDLIEKILPERVKDENSLNALSCFKGFTTYFTDYYKNRKNIYSDEEKHSTVGYRCINENLLIFMSNIEVYQIYKKANIKNDNYDEETLDKTFMIESFNECLTQSGVEAYNSVVASIKTATNLYIQKNNKEENFVRVPKMKVLFKQILSDRTSLFDGLIIESDDELLDKLCSFSAEVDKFLPINIDRYIKTLMDSNNGTGIYVKNDSSLTTLSNYLTDSWSSIRNAFNENYDAKYTGKVNDKYEEKREKAYKSNDSFELNYIQNLLGINVIDKYIERINFDIKEICEAYKEMTKNCFEDHDKTKKLQKNIKAVASIKSYLDSLKNIERDIKLLNGTGLESRNEFFYGEQSTVLEEITKVDELYNITRNYLTKKPFSTEKMKLNFNNPQLLGGWDVNKERDCYGVILIKDNNYYLGIMDKSANKSFLNIKESKNENAYKKVNCKLLPGPNKMFPKVFFAKSNIDYYDPTHEIKKLYDKGTFKKGNSFNLEDCHKLIDFYKESIKKNDDWKNFNFNFSDTKDYEDISGFFREVEAQNYKITYTNVSCDFIESLVDEGKLYLFQIYNKDFSEYATGNLNLHTLYLKMLFDERNLKDLCIKMNGEAEVFYRPASILDEDKVVHKANQKITNKNTNSKKKESIFSYDIVKDKRYTVDKFFIHLPITLNYKEQNVSRFNDYIREILKKSKNIRVIGIDRGERNLLYVVVCDSDGSILYQRSINEIVSGSHKTDYHKLLDNKEKERLSSRRDWKTIENIKDLKAGYMSQVVNEIYNLILKYNAIVVLEDLNIGFKNGRKKVEKQVYQNFEKALIDKLNYLCIDKTREQLSPSSPGGVLNAYQLTAKFESFEKIGKQTGCIFYVPAYLTSQIDPTTGFVNLFYQKDTSKQGLQLFFRKFKKINFDKVASNFEFVFDYNDFTNKAEGTKTNWTISTQGTRIAKYRSDDANGKWISRTVHPTDIIKEALNREKINYNDGHDLIDEIVSIEKSAVLKEIYYGFKLTLQLRNSTLANEEEQEDYIISPVKNSSGNYFDSRITSKELPCDADANGAYNIARKGLWALEQIRNSENVSKVKLAISNKEWFEYTQNNIPSL

SEQ ID NO: 167 - protein, 1222 aa, Oribacterium sp.
METEILKYDFFEREGKYMYYDGLTKQYALSKTIRNELVPIGKTLDNIKKNRILEADIKRKSDYEHVKKLMDMYHKKIINEALDNFKLSVLEDAADIYFNKQNDERDIDAFLKIQDKLRKEIVEQLKGHTDYSKVGNKDFLGLLKAASTEEDRILIESFDNFYTYFTSYNKVRSNLYSAEDKSSTVAYRLINENLPKFFDNIKAYRTVRNAGVISGDMSIVEQDELFEVDTFNHTLTQYGIDTYNHMIGQLNSAINLYNQKMHGAGSFKKLPKMKELYKQLLTEREEEFIEEYTDDEVLITSVHNYVSYLIDYLNSDKVESFFDTLRKSDGKEVFIKNDVSKTTMSNILFDNWSTIDDLINHEYDSAPENVKKTKDDKYFEKRQKDLKKNKSYSLSKIAALCRDTTILEKYIRRLVDDIEKIYTSNNVFSDIVLSKHDRSKKLSKNTNAVQAIKNMLDSIKDFEHDVMLINGSGQEIKKNLNVYSEQEALAGILRQVDHIYNLTRNYLTKKPFSTEKIKLNFNRPTFLDGWDKNKEEANLGILLIKDNRYYLGIMNTSSNKAFVNPPKAISNDIYKKVDYKLLPGPNKMLPKVFFATKNIAYYAPSEELLSKYRKGTHKKGDSFSIDDCRNLIDFFKSSINKNTDWSTFGFNFSDTNSYNDISDFYREVEKQGYKLSFTDIDACYIKDLVDNNELYLFQIYNKDFSPYSKGKLNLHTLYFKMLFDQRNLDNVVYKLNGEAEVFYRPASIESDEQIIHKSGQNIKNKNQKRSNCKKTSTFDYDIVKDRRYCKDKFMLHLPITVNFGTNESGKFNELVNNAIRADKDVNVIGIDRGERNLLYVVVVDPCGKIIEQISLNTIVDKEYDIETDYHQLLDEKEGSRDKARKDWNTIENIKELKEGYLSQVVNIIAKLVLKYDAIICLEDLNFGFKRGRQKVEKQVYQKFEKMLIDKMNYLVLDKSRKQESPQKPGGALNALQLTSAFKSFKELGKQTGIIYYVPAYLTSKIDPTTGFANLFYIKYESVDKARDFFSKFDFIRYNQMDNYFEFGFDYKSFTERASGCKSKWIACTNGERIVKYRNSDKNNSFDDKTVILTDEYRSLFDKYLQNYIDEDDLKDQILQIDSADFYKNLIKLFQLTLQMRNSSSDGKRDYIISPVKNYREEFFCSEFSDDTFPRDADANGAYNIARKGLWVIKQIRETKSGTKINLAMSNSEWLEYAQCNLL

SEQ ID NO: 168 - protein, 1205 aa, Pseudobutyrivibrio ruminis
MYYQNLTKMYPISKTLRNELIPVGKTLENIRKNGILEADIQRKADYEHVKKLMDNYHKQLINEALQGVHLSDLSDAYDLYFNLSKEKNSVDAFSKCQDKLRKEIVSLLKNHENFPKIGNKEIIKLLQSLYDNDTDYKALDSFSNFYTYFSSYNEVRKNLYSDEEKSSTVAYRLINENLPKFLDNIKAYAIAKKAGVRAEGLSEEDQDCLFIIETFERTLTQDGIDNYNAAIGKLNTAINLFNQQNKKQEGFRKVPQMKCLYKQILSDREEAFIDEFSDDEDLITNIESFAENMNVFLNSEIITDFKIALVESDGSLVYIKNDVSKTSFSNIVFGSWNAIDEKLSDEYDLANSKKKKDEKYYEKRQKELKKNKSYDLETIIGLFDDNSDVIGKYIEKLESDITAIAEAKNDFDEIVLRKHDKNKSLRKNTNAVEAIKSYLDTVKDFERDIKLINGSGQEVEKNLVVYAEQENILAEIKNVDSLYNMSRNYLTQKPFSTEKFKLNFNRATLLNGWDKNKETDNLGILFEKDGMYYLGIMNTKANKIFVNIPKATSNDVYHKVNYKLLPGPNKMLPKVFFAQSNLDYYKPSEELLAKYKAGTHKKGDNFSLEDCHALIDFFKASIEKHPDWSSFGFEFSETCTYEDLSGFYREVEKQGYKITYTDVDADYITSLVERDELYLFQIYNKDFSPYSKGNLNLHTIYLQMLFDQRNLNNVVYKLNGEAEVFYRPASINDEEVIIHKAGEEIKNKNSKRAVDKPTSKFGYDIIKDRRYSKDKFMLHIPVTMNFGVDETRRFNDVVNDALRNDEKVRVIGIDRGERNLLYVVVVDTDGTILEQISLNSIINNEYSIETDYHKLLDEKEGDRDRARKNWTTIENIKELKEGYLSQVVNVIAKLVLKYNAIICLEDLNFGFKRGRQKVEKQVYQKFEKMLIDKLNYLVIDKSRKQDKPEEFGGALNALQLTSKFTSFKDMGKQTGIIYYVPAYLTSKIDPTTGFANLFYVKYENVEKAKEFFSRFDSISYNNESGYFEFAFDYKKFTDRACGARSQWTVCTYGERIIKFRNTEKNNSFDDKTIVLSEEFKELFSIYGISYEDGAELKNKIMSVDEADFFRSLTRLFQQTMQMRNSSNDVTRDYIISPIMNDRGEFFNSEACDASKPKDADANGAFNIARKGLWVLEQIRNTPSGDKLNLAMSNAEWLEYAQRNQI

SEQ ID NO: 169 - protein, 1154 aa, Proteocatella sphenisci
MENFKNLYPINKTLRFELRPYGKTLENFKKSGLLEKDAFKANSRRSMQAIIDEKFKETIEERLKYTEFSECDLGNMTSKDKKITDKAATNLKKQVILSFDDEIFNNYLKPDKNIDALFKNDPSNPVISTFKGFTTYFVNFFEIRKHIFKGESSGSMAYRIIDENLTTYLNNIEKIKKLPEELKSQLEGIDQIDKLNNYNEFITQSGITHYNEIIGGISKSENVKIQGINEGINLYCQKNKVKLPRLTPLYKMILSDRVSNSFVLDTIENDTELIEMISDLINKTEISQDVIMSDIQNIFIKYKQLGNLPGISYSSIVNAICSDYDNNFGDGKRKKSYENDRKKHLETNVYSINYISELLTDTDVSSNIKMRYKELEQNYQVCKENFNATNWMNIKNIKQSEKTNLIKDLLDILKSIQRFYDLFDIVDEDKNPSAEFYTWLSKNAEKLDFEFNSVYNKSRNYLTRKQYSDKKIKLNFDSPTLAKGWDANKEIDNSTIIMRKFNNDRGDYDYFLGIWNKSTPANEKIIPLEDNGLFEKMQYKLYPDPSKMLPKQFLSKIWKAKHPTTPEFDKKYKEGRHKKGPDFEKEFLHELIDCFKHGLVNHDEKYQDVFGFNLRNTEDYNSYTEFLEDVERCNYNLSFNKIADTSNLINDGKLYVFQIWSKDFSIDSKGTKNLNTIYFESLFSEENMIEKMFKLSGEAEIFYRPASLNYCEDIIKKGHHHAELKDKFDYPIIKDKRYSQDKFFFHVPMVINYKSEKLNSKSLNNRTNENLGQFTHIIGIDRGERHLIYLTVVDVSTGEIVEQKHLDEIINTDTKGVEHKTHYLNKLEEKSKTRDNERKSWEAIETIKELKEGYISHVINEIQKLQEKYNALIVMENLNYGFKNSRIKVEKQVYQKFETALIKKFNYIIDKKDPETYIHGYQLTNPITTLDKIGNQSGIVLYIPAWNTSKIDPVTGFVNLLYADDLKYKNQEQAKSFIQKIDNIYFENGEFKFDIDFSKWNNRYSISKTKWTLTSYGTRIQTFRNPQKNNKWDSAEYDLTEEFKLILNIDGTLKSQDVETYKKFMSLFKLMLQLRNSVTGTDIDYMISPVTDKTGTHFDSRENIKNLPADADANGAYNIARKGIMAIENIMNGISDPLKISNEDYLKYIQNQQE

SEQ ID NO: 170 - protein, 1171 aa, Proteocatella sphenisci
MIILYISTSNMNMEGVFMENFKNLYPINKTLRFELRPYGKTLENFKKSGLLEKDAFKANSRRSMQAIIDEKFKETIEERLKYTEFSECDLGNMTSKDKKITDKAATNLKKQVILSFDDEIFNNYLKPDKNIDALFKNDPSNPVISTFKGFTTYFVNFFEIRKHIFKGESSGSMAYRIIDENLTTYLNNIEKIKKLPEELKSQLEGIDQIDKLNNYNEFITQSGITHYNEIIGGISKSENVKIQGINEGINLYCQKNKVKLPRLTPLYKMILSDRVSNSFVLDTIENDTELIEMISDLINKTEISQDVIMSDIQNIFIKYKQLGNLPGISYSSIVNAICSDYDNNFGDGKRKKSYENDRKKHLETNVYSINYISELLTDTDVSSNIKMRYKELEQNYQVCKENFNATNWMNIKNIKQSEKTNLIKDLLDILKSIQRFYDLFDIVDEDKNPSAEFYTWLSKNAEKLDFEFNSVYNKSRNYLTRKQYSDKKIKLNFDSPTLAKGWDANKEIDNSTIIMRKFNNDRGDYDYFLGIWNKSTPANEKIIPLEDNGLFEKMQYKLYPDPSKMLPKQFLSKIWKAKHPTTPEFDKKYKEGRHKKGPDFEKEFLHELIDCFKHGLVNHDEKYQDVFGFNLRNTEDYNSYTEFLEDVERCNYNLSFNKIADTSNLINDGKLYVFQIWSKDFSIDSKGTKNLNTIYFESLFSEENMIEKMFKLSGEAEIFYRPASLNYCEDIIKKGHHHAELKDKFDYPIIKDKRYSQDKFFFHVPMVINYKSEKLNSKSLNNRTNENLGQFTHIIGIDRGERHLIYLTVVDVSTGEIVEQKHLDEIINTDTKGVEHKTHYLNKLEEKSKTRDNERKSWEAIETIKELKEGYISHVINEIQKLQEKYNALIVMENLNYGFKNSRIKVEKQVYQKFETALIKKFNYIIDKKDPETYIHGYQLTNPITTLDKIGNQSGIVLYIPAWNTSKIDPVTGFVNLLYADDLKYKNQEQAKSFIQKIDNIYFENGEFKFDIDFSKWNNRYSISKTKWTLTSYGTRIQTFRNPQKNNKWDSAEYDLTEEFKLILNIDGTLKSQDVETYKKFMSLFKLMLQLRNSVTGTDIDYMISPVTDKTGTHFDSRENIKNLPADADANGAYNIARKGIMAIENIMNGISDPLKISNEDYLKYIQNQQE

SEQ ID NO: 171 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 172 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDMKDSLKEMSLDEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSRFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCRDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKENDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFANIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 173 - protein, 1263 aa, Eubacterium rectale
MNNGTNNFQNFIGISSLQKTLRNALTPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDMKDSLKKMSLEKIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPEKKIIEGNTSENKGDYKKMIYNLLPGPNKMIPKVFLSSKTGVETYKPSAYILEGYKQNKHLKSSKDFDITFCRDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDVVLKLNGEAEIFFRKSSIKNPIIHKKGSILVNRTYEAEEKDQFGNIQIVRKTIPENIYQELYKYFNDKSDKELSDEAAKLKNAVGHHEAATNIVKDYRYTYDKYFLHMPITINFKANKTSFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFVNIFKFKDLTVDAKREFIKKFDSIRYDSDKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRNYDRLISPVLNENNIFYDSAKAGDALPKDADANGAYCIALKGLYEIKQITENWKEDGKFSRDKLKISNKDWFDFIQNKRYL

SEQ ID NO: 174 - protein, 1263 aa, Eubacterium sp.
MNNGTNNFQNFIGISSLQKTLRNALIPTETTQQFIVKNGIIKEDELRGKNRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKKEKTQVIKLFSRFATSFKDYFKNRANCFSADDISSSSCHRIVNDNAEIFFSNALVYRRIVKNLSNDDINKISGDMKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNDYNLDKIYIVSKFYESVSQKTYRDWETINTALEIHYNNILPGNGKSKADKVKKAVKNDLQKSITEINELVSNYKLCSDDNIKAETYIHEISHILNNFEAHELKYNPEIHLVESELKASELKNVLDIIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSAYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFKLTVQMRNSLSELEDRDYDRLISPVLNENNIFYDSAKAGYALPKDADANGAYCIALKGLYEIKQITENWKEDGKFSRDKLKISNKDWFDFIQNKRYL

SEQ ID NO: 175 - protein, 1145 aa, Alicyclobacillus macrosporangiidus
MVAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 176 - protein, 1108 aa, unidentified
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 177 - protein, 1364 aa, unidentified
MPRDDLDLLTNLNSTAKGIRERGKTKEGTDKKKSGRKSSWPMDKAAWETAKTSDSSAHFLEKLKQHPDLKDAFGNLSSGGSKKLEYYKKLAGSAPWKESQSVILEKAARWKEAKQEREEKEQDSSEHGSKAAYRRLFDAGCLPMPEFAKYIDENQIEFGDLKLSDCGAEWKRGMWNQAGQRVRSHMGWQRRREKENAVYSLRKELFEKGGAIRRKKSEELTPEDILPGKAAPDQNDWQERPAYGNQMWFIGLRSYEENEMAKYAEEAGMGSRSAPRIRRGTIKGWSKLRERWLQILKRNPQATRDDLIGELNALRSQDPRAYGDARLFDWLSKTDQRFLWDGFDADGKILCGRDDRDCVSAFVAYNEEFADEPSSITLTETDERLHPVWPFFGESSAVPYEIEYDLETACPTAIRLPLLVGKENGGYAERQGTRLPLAEYADLASSFQLPTPVRLDVLVEIREVTRAGRKVTCPFSYFKQNGVWYVREGEIPSGESIQIKQTDRKIENGKIFISSKLRMAYRDDLMVSPATGDFGSIKILWERIELASHVDQKKLPETAPARSRVFVSFSCNVVERAPRKQLTRKPDAVVVTIPSGVDQGLVVVSTDVRTGKSKSSSAPPLPPGSRLWPADAVHGDPPLRILSVDLGHRHSAYAVWELGLQQKSWRAGVLKGSTQTPVYADCTGTGLLCLPGDGEDTPAEEESLRLRSRQIRRRLNLQNSILRVSRLLSLDKFEKTIFEQSDVRDRPNKKGLRIRRRCRTEKTPLSEAEVRKNCDKAAEILIRWADTDAMAKSLAATGNADISFWKYMAVKNPPLSAVVDVAPSTIVPDDGPDRETLKKKRQEEEEKFASSIYENRVKLAGALCSGYDADHRRPATGGLWHDLDRTLIREISYGDRGQKGNPRKLNNEGILRLLRRPPRARPDWREFHRTLNDANRIPKGRTLRGGLSMGRLNFLKEVGDFVKKWSCRPRWPGDRRHIPPGQLFDRQDAEHLEHLRDDRIKRLAHLIVAQALGFEPDIRRGLWKYVDGSTGEILWQHPETRRFFAEGAAGELREVSRPAEIDDDAAARPHTVSAPAHIVVFENLIRYRFQSDRPKTENAGLMQWAHRQIVHFTKQVASLYGLKVAMVYAAFSSKFCSRCGSPGARVSRFDPAWRNQEWFKRRTSNPRSKVDHSLKRASEDPTADETRPWVLIEGGKEFVCANAKCSAHDEPLNADENAAANIGLRFLRGVEDFRTKVNPAGALKGKLRFETGIHSFRPPVSGSPFWSPMAEPAQKKKIGAAAPGADVDEAGDADESGVVVLFRDPSGAFRNKQYWYEGKIFWSNVMMAVEAKIAGASVGAKPVAASWGQAQPQSGPGLAKPGGD

SEQ ID NO: 178 - protein, 1364 aa, unidentified
MNRIYQGRVTKVEVPDGKDEKGNIKWKKLENWSDILWQHHMLFQDAVNYYTLALAAISGSAVGSDEKSIILREWAVQVQNIWEKAKKKATVFEGPQKRLTSILGLEQNASFDIAAKHILRTSEAKPEQRASALIRLLEEIDKKNHNVVCGERLPFFCPRNIQSKRSPTSKAVSSVQEQKRQEEVRRFHNMQPEEVVKNAVTLDISLFKSSPKIVFLEDPKKARAELLKQFDNACKKHKELVGIKKAFTESIDKHGSSLKVPAPGSKPSGLYPSAIVFKYFPVDITKTVFLKATEKLAMGKDREVTNDPIADARVNDKPHFDYFTNIALIREKEKNRAAWFEFDLAAFIEAIMSPHRFYQDTQKRKEAARKLEEKIKAIEGKGGQFKESDSEDDDVDSLPGFEGDTRIDLLRKLVTDTLGWLGESETPDNNEGKKTEYSISERTLRIFPDIQKQWSELAEKGETTEGKLLEVLKHEQTEHQSDFGSATLYQHLAKPEFHPIWLKSGTEEWHAENPLKAWLNYKELQYELTDKKRPIHFTPAHPVYSPRYFDFPKKSETEEKEVSKNTHSLTTSLASEHIKNSLQFTAGLIRKTNVGKKAIKARFSYSAPRLRRDCLRSENNENLYKAPWLQPMMRALGIDEEKADRQNFANTRITLMAKGLDDIQLGFPVEANSQELQKEVSNGISWKGQFNWGGIASLSALRWPHEKKPKNPPEQPWWGIDSFSCLAVDLGQRYAGAFARLDVSTIEKKGKSRFIGEACDKKWYAKVSRMGLLRLPGEDVKVWRDASKIDKENGFAFRKELFGEKGRSATPLEAEETAELIKLFGANEKDVMPDNWSKELSFPEQNDKLLIVARRAQAAVSRLHRWAWFFDEAKRSDDAIREILESDDTDLKQKVNKNEIEKVKETIISLLKVKQELLPTLLTRLANRVLPLRGRSWEWKKHHQKNDGFILDQTGKAMPNVLIRGQRGLSMDRIEQITELRKRFQALNQSLRRQIGKKAPAKRDDSIPDCCPDLLEKLDHMKEQRVNQTAHMILAEALGLKLAEPPKDKKELNETCDMHGAYAKVDNPVSFIVIEDLSRYRSSQGRSPRENSRLMKWCHRAVRDKLKEMCEVFFPLCERRKAGSAWVSLPPLLETPAAYSSRFCSRSGVAGFRAVEVIPGFELKYPWSWLKDKKDKAGNLAKEALNIRTVSEQLKAFNQDKPEKPRTLLVPIAGGPIFVPISEVGLSSFGLKPQVVQADINAAINLGLRAISDPRIWEIHPRLRTEKRDGRLFAREKRKYGEEKVEVQPSKNEKAKKVKDDRKPNYFADFSGKVDWGFGNIKNESGLTLVSGKALWWTINQLQWERCFDINKRHIEDWSNKQKQ

SEQ ID NO: 179 - protein, 1413 aa, unidentified
MNRIYQGRVTKVEKLKNGKSPDDREELKDWQTALWRHHELFQDAVSYYTLALAAMAEGLPDKHPINVLRKRMEEAWEEFPRKTVTPAKNLRDSVRPWLGLSESASFGDALKKILPPAPENKEVRALAVALLAEKARTLKPQKTSASYWGRFCDDLKKKPNWDYSEEELARKTGSGDWVAGLWSEDALNKIDELAKSLKLSSLVKCVPDGQINPEGARNLVKEALDHLEGVSNGTKKEKNDPGPAKKTNNWLRQHASDVRNFIHKNKNQFSSLPNGRLITERARGGGININKTYAGVLFKAFPCPFTFDYVRAAVPEPKVKKVDQEKKSEQSATWTELEKRILRIGDDPIELARKNNKPIFKAFTALEKWSDQNSKSCWSDFDKCAFEEALKTLNQFNQKTEEREKRRSEAEAELKYMMDENPEWKPKKETEGDDVREVPILKGDPRYEKLVKLFGDLDEEGSEHATGKIYGPSRASLRGFGKLRNEWVDLFTKANDNPREQDLQKAVTGFQREHKLDMGYTAFFLKLCERDYWDIWRDDTEVEVKKIREKRWVKSVVYAAADTRELAEELERLQEPVRYTPAEPQFSRRLFMFSDIKGKQGAKHIREGLVEVSLAVKDQSGKYGTCRVRLHYSAPRLIRDHLSDGSSSMWLQPMMAALGLSSDARGCFTRDSKGNVKEPAVALMSDFVGRKRELRMLLNFPVDLDISKLEENIGKKARWEKQMNTAYEKNKLKQRFHLIWPGMELKETQEPGQFWWDNPTIQKEGMYCLAIDLSQRRAADYALLHAGVNRDSKTFVELGQAGGQSWFTKLCAAGSLRLPGEDTEVIREGKRQIELSGKKGRNATQSEYDQAIALAKQLLHNENSAELESAARDWLGDNAKRFSFPEQNDKLIDLYYGALSRYKTWLRWSWRLTEQHKELWDKTLDEIRKVPYFASWGELAGNGTNEATVQQLQKLIADAAVDLRNFLEKALLHIAYRALPLRENTWRWIENGKDGKGKPLHLLVSDGQSPAEIPWLRGQRGLSIARIEQLENFRRAVLSLNRLLRHEIGTKPEFGSSTCGESLPDPCPDLTDKIVRLKEERVNQTAHLIIAQSLGVRLKGHSLFTEEREKADMHGEHEVIPGRSPVDFVVLEDLSRYTTDKSRSRSENSRLMKWCHRKINEKVKLLAEPFGIPVIEVFASYSSKFDARTGAPGFRAVEVTSEDRPFWRKTIEKQSVAREVFDCLDNLVGKGLNGIHLVLPQNGGPLFIAAVKEDQPLPAIRQADINAAVNIGLRAIAGPSCYHAHPKVRLIKGESGTDKGKWLPRKGKEANKRENAQFGNVDLDLEVKFNRLDIDSDVLKGDNTNLFHDPLNIACYGFATIQNLQHPFLAHASAVFSRQKGAVARLQWEVCRAINSRRLEAWQKKAEKAAVKR

SEQ ID NO: 180 - protein, 747 aa, unidentified
MATKSYRARILTDSRLAAALDRTHVVFVESLKQMINTYLRMQNGKFGPDHKKLAQIMLSRSNTFAHGVMDQITRDQPTSTLDEEWTDLARRIHKTTGPLFLQAERFATVKNRAIHTKSRGKVIPSPETLAVPAKFWHQVCDSASAYIRSNRELMQQWRKDRAAWLKDKNEWQQKHPEFMQFYNGPYQNFLKLCDDDRITSQLAAEQQPTASKNNRPRKTGKRFARWHLWYKWLSENPEIIEWRNKASASDFKTVTDDVRKQIITKYPQQNKYITRLLDWLEDNNPELKTLENLRRTYVKKFDSFKRPPTLTLPSPYRHPYWFTMELDQFYKKADFENGTIQLLLIDEDDDGNWFFNWMPASLKPDPRLVPSWRAETFETEGRFPPYLGGKIGKKLSRPAPTDAERKAGIAGAKLMIKNNRSELLFTVFEQDCPPRVKWAKTKNRKCPADNAFSSDGKTRKPLRILSIDLGIRHIGAFALTQGTRNDSAWQTESLKKGIINSPSIPPLRQVRRHDYDLKRKRRRHGKPVKGQRSNANLQAHRTNMAQDRFKKGASAIVSLAREHSADLILFENLHSLKFSAFDERWMNRQLRDMNRRHIVELVSEQAPEFGITVKDDINPWMTSRICSNCNLPGFRFSMKKKNPYREKLPREKCTDFGYPVWEPGGHLFRCPHCDHRVNADINAAANLANKFFGLGYWNNGLKYDAETKTFTVHTDKKTPPLIFKPRPQFDLWADSVKTRKQLGPDPF

SEQ ID NO: 181 - protein, 743 aa, unidentified
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 182 - protein, 1119 aa, unidentified
MSFTISYPFKLIIKNKDEAKALLDTHQYMNEGVKYYLEKLLMFRQEKIFIGEDETGKRIYIEETEYKKQIEEFYLIKKTELGRNLTLTLDEFKTLMRELYICLVSSSMENKKGFPNAQQASLNIFSPLFDAESKGYILKEENNNISLIHKDYGKILLKRLRDNNLIPIFTKFTDIKKITAKLSPTALDRMIFAQAIEKLLSYESWCKLMIKERFDKEVKIKELENKCENKQERDKIFEILEKYEEERQKTFEQDSGFAKKGKFYITGRMLKGFDEIKEKWLKEKDRSEQNLINILNKYQTDNSKLVGDRNLFEFIIKLENQCLWNGDIDYLKIKRDINKNQIWLDRPEMPRFTMPDFKKHPLWYRYEDPSNSNFRNYKIEVVKDENYITIPLITERNNEYFEENYTFNLAKLKKLSENITFIPKSKNKEFEFIDSNDEEEDKKDQKKSKQYIKYCDTAKNTSYGKSGGIRLYFNRNELENYKDGKKMDSYTVFTLSIRDYKSLFAKEKLQPQIFNTVDNKITSLKIQKKFGNEEQTNFLSYFTQNQITKKDWMDEKTFQNVKELNEGIRVLSVDLGQRFFAAVSCFEIMSEIDNNKLFFNLNDQNHKIIRINDKNYYAKHIYSKTIKLSGEDDDLYKERKINKNYKLSYQERKNKIGIFTRQINKLNQLLKIIRNDEIDKEKFKELIETTKRYVKNTYNDGIIDWNNVDNKILSYENKEDVINLHKELDKKLEIDFKEFIRECRKPIFRSGGLSMQRIDFLEKLNKLKRKWVARTQKSAESIVLTPKFGYKLKEHINELKDNRVKQGVNYILMTALGYIKDNEIKNDSKKKQKEDWVKKNRACQIILMEKLTEYTFAEDRPREENSKLRMWSHRQIFNFLQQKASLWGILVGDVFAPYTSKCLSDNNAPGIRCHQVTKKDLIDNSWFLKIVVKDDAFCDLIEINKENVKNKSIKINDILPLRGGELFASIKDGKLHIVQADINASRNIAKRFLSQINPFRVVLKKDKDETFHLKNEPNYLKNYYSILNFVPTNEELTFFKVEENKDIKPTKRIKMDKHEKESTDEGDDYSKNQIALFRDDSGIFFDKSLWVDGKIFWSVVKNKMTKLLRERNNKKNGSK

SEQ ID NO: 183 - protein, 1447 aa, unidentified
MPLSRIYQGRTNSLIILTPTPQEPWDHKALARFDSPLWRHHALFQDAVNYYQLCLVALASSDGTRPLSKLHEQMKASWDEAKTDTEDSWRVRLARRLGIPAASLFEAALAKVLEGNEAPERARELAGELLLDKIEGDIQQAGRGYWPRFCDPKANPTYDYSATARASASGLTKLAAVIHAENVTEEALKQVAAEMDLSWTVKLQPDKNFVGAEARARLLEAAHHFIKVAESPPTKLAEVLARFPDGLALWQALPEKIAALPEETQVPRNRKASPDLTFATLLFQHFPSLFTAAVLGLSVGKPKSVKAPKVVEKVSARRKANAVTQAVVIEEPEIDFAELGDDPIKLARGERGFVFPAFTSLSFWAVPGPHVPVWKEFDIAAFKEALKTVNQFKLKTSERNALLAEAQRRLDYMDEKTHDWKTGDSDEPGHIPPRLKSDPNFTLIQALTQDEGVSNKATGDQHIPKGVYTGGLRGFYAIKKDWCELWERKADKSQGTPTEEELISIVTDYQRDHVYDVGDVGLFRALCEPRFWPLWQPLTDEQEAERIKAGRAKDMISAYRVWLELQEDVVRLAQPIRFTPAHAENSRRLFMFSDISGSHGAEFGSDGKSLEVSIAYDVDGKLQPVRAKLEFSAPRAARDELEGLSGGSESMRWFQPMMKALDCPEVEMPALEKCAVSLMPDVVKKGGGKWVRLLLNFPATLEPEGLIRHIGKQAMWYKQFNGTYKPRTQQLDTGLHLYWPGLEKAPEAEDAAAWWNREEIRAKGFSVLSVDLGQRDAGAWALLESRSDKAFSRNRQPFIELGEAGGKLWSTALLGLGMLRLPGEDARTGALDDQGKRAVEFHGKAGRNALEAEWQEAREMALLFGGEEAKSRLGPGFDHLSHSKQNEELLRILSRAQSRLARFHRWSCRIHEKPEATGDDVIDYGQVDELLTKTAEAMLENLKALYTNAGGILDSKSKQPLTLVGLRKKLEAQKVEPEKIAAVLKPHAEIIFQRLGTLIPELKQHLRVSLERLANRELPLRHREWVWNEAFEKLEQGNFKKEENPKWIRGQRGLSMARIEQIENLRKRFMSLRRQMSLIPGEQVKQGVEDKGQRQPEPCEDILNKLDRMKQQRVNQTAHLILAQALGLRLRPHLANDAEREEKDIHGEYELIPGRKPVDFIVMEDLSRYLSSQGRAPSENGRLMKWCHRAVLAKLKQMCEPFGIPVLEVPAAYSSRFCALTGVPGFRAVEVHDGNAEDFRWKRLIKKAEKDKSSKDAEAAAMLFDQLHDLNIEAREARKQDKKLPLRTLFAPVAGGPLFIPMVGGGPRQADMNAAINLGLRAIASPTCLRARPKIRAELKDGKHQAMLGNKLEKAAALTLEPPKEPTKELAAQKRTNFFLDEKFVGKFDTAHVTTSGKKLRLSGGMSLWKAIKDGAWQRVKKINDARIAKWKNNPPPEPDPDDEIQF

SEQ ID NO: 184 - protein, 1147 aa, Alicyclobacillus kakegawensis
MAVKSIKVKLRLSECPDILAGMWQLHRATNAGVRYYTEWVSLMRQEILYSRGPDGGQQCYMTAEDCQRELLRRLRNRQLHNGRQDQPGTDADLLAISRRLYEILVLQSIGKRGDAQQIASSFLSPLVDPNSKGGRGEAKSGRKPAWQKMRDQGDPRWVAAREKYEQRKAVDPSKEILNSLDALGLRPLFAVFTETYRSGVDWKPLGKSQGVRTWDRDMFQQALERLMSWESWNRRVGEEYARLFQQKMKFEQEHFAEQSHLVKLARALEADMRAASQGFEAKRGTAHQITRRALRGADRVFEIWKSIPEEALFSQYDEVIRQVQAEKRRDFGSHDLFAKLAEPKYQPLWRADETFLTRYALYNGVLRDLEKARQFATFTLPDACVNPIWTRFESSQGSNLHKYEFLFDHLGPGRHAVRFQRLLVVESEGAKERDSVVVPVAPSGQLDKLVLREEEKSSVALHLHDTARPDGFMAEWAGAKLQYERSTLARKARRDKQGMRSWRRQPSMLMSAAQMLEDAKQAGDVYLNISVRVKSPSEVRGQRRPPYAALFRIDDKQRRVTVNYNKLSAYLEEHPDKQIPGAPGLLSGLRVMSVDLGLRTSASISVFRVAKKEEVEALGDGRPPHYYPIHGTDDLVAVHERSHLIQMPGETETKQLRKLREERQAVLRPLFAQLALLRLLVRCGAADERIRTRSWQRLTKQGREFTKRLTPSWREALELELTRLEAYCGRVPDDEWSRIVDRTVIALWRRMGKQVRDWRKQVKSGAKVKVKGYQLDVVGGNSLAQIDYLEQQYKFLRRWSFFARASGLVVRADRESHFAVALRQHIENAKRDRLKKLADRILMEALGYVYEASGPREGQWTAQHPPCQLIILEELSAYRFSDDRPPSENSKLMAWGHRGILEELVNQAQVHDVLVGTVYAAFSSRFDARTGAPGVRCRRVPARFVGATVDDSLPLWLTEFLDKHRLDKNLLRPDDVIPTGEGEFLVSPCGEEAARVRQVHADINAAQNLQRRLWQNFDITELRLRCDVKMGGEGTVLVPRVNNARAKQLFGKKVLVSQDGVTFFERSQTGGKPHSEKQTDLTDKELELIAEADEARAKSVVLFRDPSGHIGKGHWIRQREFWSLVKQRIESHTAERIRVRGVGSSLD

SEQ ID NO: 185 - protein, 1112 aa, Bacillus sp.
MAIRSIKLKMKTNSGTDSIYLRKALWRTHQLINEGIAYYMNLLTLYRQEAIGDKTKEAYQAELINIIRNQQRNNGSSEEHGSDQEILALLRQLYELIIPSSIGESGDANQLGNKFLYPLVDPNSQSGKGTSNAGRKPRWKRLKEEGNPDWELEKKKDEERKAKDPTVKIFDNLNKYGLLPLFPLFTNIQKDIEWLPLGKRQSVRKWDKDMFIQAIERLLSWESWNRRVADEYKQLKEKTESYYKEHLTGGEEWIEKIRKFEKERNMELEKNAFAPNDGYFITSRQIRGWDRVYEKWSKLPESASPEELWKVVAEQQNKMSEGFGDPKVFSFLANRENRDIWRGHSERIYHIAAYNGLQKKLSRTKEQATFTLPDAIEHPLWIRYESPGGTNLNLFKLEEKQKKNYYVTLSKIIWPSEEKWIEKENIEIPLAPSIQFNRQIKLKQHVKGKQEISFSDYSSRISLDGVLGGSRIQFNRKYIKNHKELLGEGDIGPVFFNLVVDVAPLQETRNGRLQSPIGKALKVISSDFSKVIDYKPKELMDWMNTGSASNSFGVASLLEGMRVMSIDMGQRTSASVSIFEVVKELPKDQEQKLFYSINDTELFAIHKRSFLLNLPGEVVTKNNKQQRQERRKKRQFVRSQIRMLANVLRLETKKTPDERKKAIHKLMEIVQSYDSWTASQKEVWEKELNLLTNMAAFNDEIWKESLVELHHRIEPYVGQIVSKWRKGLSEGRKNLAGISMWNIDELEDTRRLLISWSKRSRTPGEANRIETDEPFGSSLLQHIQNVKDDRLKQMANLIIMTALGFKYDKEEKDRYKRWKETYPACQIILFENLNRYLFNLDRSRRENSRLMKWAHRSIPRTVSMQGEMFGLQVGDVRSEYSSRFHAKTGAPGIRCHALTEEDLKAGSNTLKRLIEDGFINESELAYLKKGDIIPSQGGELFVTLSKRYKKDSDNNELTVIHADINAAQNLQKRFWQQNSEVYRVPCQLARMGEDKLYIPKSQTETIKKYFGKGSFVKNNTEQEVYKWEKSEKMKIKTDTTFDLQDLDGFEDISKTIELAQEQQKKYLTMFRDPSGYFFNNETWRPQKEYWSIVNNIIKSCLKKKILSNKVEL

SEQ ID NO: 186 - protein, 1489 aa, Desulfatirhabdium butyrativorans
MPLSNNPPVTQRAYTLRLRGADPSDLSWREALWHTHEAVNKGAKVFGDWLLTLRGGLDHTLADTKVKGGKGKPDRDPTPEERKARRILLALSWLSVESKLGAPSSYIVASGDEPAKDRNDNVVSALEEILQSRKVAKSEIDDWKRDCSASLSAAIRDDAVWVNRSKVFDEAVKSVGSSLTREEAWDMLERFFGSRDAYLTPMKDPEDKSSETEQEDKAKDLVQKAGQWLSSRYGTSEGADFCRMSDIYGKIAAWADNASQGGSSTVDDLVSELRQHFDTKESKATNGLDWIIGLSSYTGHTPNPVHELLRQNTSLNKSHLDDLKKKANTRAESCKSKIGSKGQRPYSDAILNDVESVCGFTYRVDKDGQPVSVADYSKYDVDYKWGTARHYIFAVMLDHAARRISLAHKWIKRAEAERHKFEEDAKRIANVPARAREWLDSFCKERSVTSGAVEPYRIRRRAVDGWKEVVAAWSKSDCKSTEDRIAAARALQDDSEIDKFGDIQLFEALAEDDALCVWHKDGEATNEPDFQPLIDYSLAIEAEFKKRQFKVPAYRHPDELLHPVFCDFGKSRWKINYDVHKNVQAPFYRGLCLTLWTGSEIKPVPLCWQSKRLTRDLALGNNHRNDAASAVTRADRLGRAASNVTKSDMVNITGLFEQADWNGRLQAPRQQLEAIAVVRDNPRLSEQERNLRMCGMIEHIRWLVTFSVKLQPQGPWCAYAEQHGLNTNPQYWPHADTNRDRKVHARLILPRLPGLRVLSVDLGHRYAAACAVWEAVNTETVKEACQNVGRDMPKEHDLYLHIKVKKQGIGKQTEVDKTTIYRRIGADTLPDGRPHPAPWARLDRQFLIKLQGEEKDAREASNEEIWALHQMECKLDRTKPLIDRLIASGWGLLKRQMARLDALKELGWIPAPDSSENLSREDGEAKDYRESLAVDDLMFSAVRTLRLALQRHGNRARIAYYLISEVKIRPGGIQEKLDENGRIDLLQDALALWHELFSSPGWRDEAAKQLWDSRIATLAGYKAPEENGDNVSDVAYRKKQQVYREQLRNVAKTLSGDVITCKELSDAWKERWEDEDQRWKKLLRWFKDWVLPSGTQANNATIRNVGGLSLSRLATITEFRRKVQVGFFTRLRPDGTRHEIGEQFGQKTLDALELLREQRVKQLASRIAEAALGIGSEGGKGWDGGKRPRQRINDSRFAPCHAVVIENLANYRPDETRTRLENRRLMTWSASKVHKYLSEACQLNGLYLCTVSAWYTSRQDSRTGAPGIRCQDVSVREFMQSPFWRKQVKQAEAKHDENKGDARERFLCELNKTWKAKTPAEWKKAGFVRIPLRGGEIFVSADSKSPSAKGIHADLNAAANIGLRALTDPDWPGKWWYVPCDPVSFESKMDYVKGCAAVKVGQPLRQPAQTNADGAASKIRKGKKNRTAGTSKEKVYLWRDISAFPLESNEIGEWKETSAYQNDVQYRVIRMLKEHIKSLDNRTGDNVEG

SEQ ID NO: 187 - protein, 1194 aa, Desulfonatronum thiodismutans
MVLGRKDDTAELRRALWTTHEHVNLAVAEVERVLLRCRGRSYWTLDRRGDPVHVPESQVAEDALAMAREAQRRNGWPVVGEDEEILLALRYLYEQIVPSCLLDDLGKPLKGDAQKIGTNYAGPLFDSDTCRRDEGKDVACCGPFHEVAGKYLGALPEWATPISKQEFDGKDASHLRFKATGGDDAFFRVSIEKANAWYEDPANQDALKNKAYNKDDWKKEKDKGISSWAVKYIQKQLQLGQDPRTEVRRKLWLELGLLPLFIPVFDKTMVGNLWNRLAVRLALAHLLSWESWNHRAVQDQALARAKRDELAALFLGMEDGFAGLREYELRRNESIKQHAFEPVDRPYVVSGRALRSWTRVREEWLRHGDTQESRKNICNRLQDRLRGKFGDPDVFHWLAEDGQEALWKERDCVTSFSLLNDADGLLEKRKGYALMTFADARLHPRWAMYEAPGGSNLRTYQIRKTENGLWADVVLLSPRNESAAVEEKTFNVRLAPSGQLSNVSFDQIQKGSKMVGRCRYQSANQQFEGLLGGAEILFDRKRIANEQHGATDLASKPGHVWFKLTLDVRPQAPQGWLDGKGRPALPPEAKHFKTALSNKSKFADQVRPGLRVLSVDLGVRSFAACSVFELVRGGPDQGTYFPAADGRTVDDPEKLWAKHERSFKITLPGENPSRKEEIARRAAMEELRSLNGDIRRLKAILRLSVLQEDDPRTEHLRLFMEAIVDDPAKSALNAELFKGFGDDRFRSTPDLWKQHCHFFHDKAEKVVAERFSRWRTETRPKSSSWQDWRERRGYAGGKSYWAVTYLEAVRGLILRWNMRGRTYGEVNRQDKKQFGTVASALLHHINQLKEDRIKTGADMIIQAARGFVPRKNGAGWVQVHEPCRLILFEDLARYRFRTDRSRRENSRLMRWSHREIVNEVGMQGELYGLHVDTTEAGFSSRYLASSGAPGVRCRHLVEEDFHDGLPGMHLVGELDWLLPKDKDRTANEARRLLGGMVRPGMLVPWDGGELFATLNAASQLHVIHADINAAQNLQRRFWGRCGEAIRIVCNQLSVDGSTRYEMAKAPKARLLGALQQLKNGDAPFHLTSIPNSQKPENSYVMTPTNAGKKYRAGPGEKSSGEEDELALDIVEQAEELAQGRKTFFRDPSGVFFAPDRWLPSEIYWSRIRRRIWQVTLERNSSGRQERAEMDEMPY

SEQ ID NO: 188 - protein, 1326 aa, unidentified
MAVELNRIYQGRVNHVYIFDENQNQVSVDNGDDLLFVHHELYQDAINYYLVALAAMALDSKDSLFGKFKMQIRAVWNDFYRNGQLRPGLKHSLIRSLGHAAELNTSNGADIAMNLILEDGGIPSEILNAALEHLAEKCTGDVSQLGKTFFPRFCDTAYHGNWDVDAKSFSEKKGRQRLVDALYSLHPVQAVQELAPEIEIGWGGVKTQTGKFFTGDEAKASLKKAISYFLQDTGKNSPELQEYFSVAGKQPLEQYLGKIDTFPEISFGRISSHQNINISNAMWILKFFPDQYSVDLIKNLIPNKKYEIGIAPQWGDDPVKLSRGKRGYTFRAFTDLAMWEKNWKVFDRAAFSDALKTINQFRNKTQERNDQLKRYCAALNWMDGESSDKKPPVEPADADAVDEAATSVLPILAGDKRWNALLQLQKELGICNDFTENELMDYGLSLRTIRGYQKLRSMMLEKEEKMRAKTADDEEISQALQEIIIKFQSSHRDTIGSVSLFLKLAEPKYFCVWHDADKNQNFASVDMVADAVRYYSYQEEKARLEEPIQITPADARYSRRVSDLYALVYKNAKECKTGYGLRPDGNFVFEIAQKNAKGYAPAKVVLAFSAPRLKRDGLIDKEFSAYYPPVLQAFLREEEAPKQSFKTTAVILMPDWDKNGKRRILLNFPIKLDVSAIHQKTDHRFENQFYFANNTNTCLLWPSYQYKKPVTWYQGKKPFDVVAVDLGQRSAGAVSRITVSTEKREHSVAIGEAGGTQWYAYRKFSGLLRLPGEDATVIRDGQRTEELSGNAGRLSTEEETVQACVLCKMLIGDATLLGGSDEKTIRSFPKQNDKLLIAFRRATGRMKQLQRWLWMLNENGLCDKAKTEISNSDWLVNKNIDNVLKEEKQHREMLPAILLQIADRVLPLRGRKWDWVLNPQSNSFVLQQTAHGSGDPHKKICGQRGLSFARIEQLESLRMRCQALNRILMRKTGEKPATLAEMRNNPIPDCCPDILMRLDAMKEQRINQTANLILAQALGLRHCLHSESATKRKENGMHGEYEKIPGVEPAAFVVLEDLSRYRFSQDRSSYENSRLMKWSHRKILEKLALLCEVFNVPILQVGAAYSSKFSANAIPGFRAEECSIDQLSFYPWRELKDSREKALVEQIRKIGHRLLTFDAKATIIMPRNGGPVFIPFVPSDSKDTLIQADINASFNIGLRGVADATNLLCNNRVSCDRKKDCWQVKRSSNFSKMVYPEKLSLSFDPIKKQEGAGGNFFVLGCSERILTGTSEKSPVFTSSEMAKKYPNLMFGSALWRNEILKLERCCKINQSRLDKFIAKKEVQNEL

SEQ ID NO: 189 - protein, 1090 aa, Laceyella sediminis
MSIRSFKLKIKTKSGVNAEELRRGLWRTHQLINDGIAYYMNWLVLLRQEDLFIRNEETNEIEKRSKEEIQGELLERVHKQQQRNQWSGEVDDQTLLQTLRHLYEEIVPSVIGKSGNASLKARFFLGPLVDPNNKTTKDVSKSGPTPKWKKMKDAGDPNWVQEYEKYMAERQTLVRLEEMGLIPLFPMYTDEVGDIHWLPQASGYTRTWDRDMFQQAIERLLSWESWNRRVRERRAQFEKKTHDFASRFSESDVQWMNKLREYEAQQEKSLEENAFAPNEPYALTKKALRGWERVYHSWMRLDSAASEEAYWQEVATCQTAMRGEFGDPAIYQFLAQKENHDIWRGYPERVIDFAELNHLQRELRRAKEDATFTLPDSVDHPLWVRYEAPGGTNIHGYDLVQDTKRNLTLILDKFILPDENGSWHEVKKVPFSLAKSKQFHRQVWLQEEQKQKKREVVFYDYSTNLPHLGTLAGAKLQWDRNFLNKRTQQQIEETGEIGKVFFNISVDVRPAVEVKNGRLQNGLGKALTVLTHPDGTKIVTGWKAEQLEKWVGESGRVSSLGLDSLSEGLRVMSIDLGQRTSATVSVFEITKEAPDNPYKFFYQLEGTELFAVHQRSFLLALPGENPPQKIKQMREIRWKERNRIKQQVDQLSAILRLHKKVNEDERIQAIDKLLQKVASWQLNEEIATAWNQALSQLYSKAKENDLQWNQAIKNAHHQLEPVVGKQISLWRKDLSTGRQGIAGLSLWSIEELEATKKLLTRWSKRSREPGVVKRIERFETFAKQIQHHINQVKENRLKQLANLIVMTALGYKYDQEQKKWIEVYPACQVVLFENLRSYRFSYERSRRENKKLMEWSHRSIPKLVQMQGELFGLQVADVYAAYSSRYHGRTGAPGIRCHALTEADLRNETNIIHELIEAGFIKEEHRPYLQQGDLVPWSGGELFATLQKPYDNPRILTLHADINAAQNIQKRFWHPSMWFRVNCESVMEGEIVTYVPKNKTVHKKQGKTFRFVKVEGSDVYEWAKWSKNRNKNTFSSITERKPPSSMILFRDPSGTFFKEQEWVEQKTFWGKVQSMIQAYMKKTIVQRMEE

SEQ ID NO: 190 - protein, 1041 aa, Methylobacterium nodulans
MYEAIVLADDANAQLANAFLGPLTDPNSAGFLEAFNKVDRPAPSWLDQVPASDPIDPAVLAEANAWLDTDAGRAWLVDTGAPPRWRSLAAKQDPIWPREFARKLGELRKEAASGTSAIIKALKRDFGVLPLFQPSLAPRILGSRSSLTPWDRLAFRLAVGHLLSWESWCTRARDEHTARVQRLEQFSSAHLKGDLATKVSTLREYERARKEQIAQLGLPMGERDFLITVRMTRGWDDLREKWRRSGDKGQEALHAIIATEQTRKRGRFGDPDLFRWLARPENHHVWADGHADAVGVLARVNAMERLVERSRDTALMTLPDPVAHPRSAQWEAEGGSNLRNYQLEAVGGELQITLPLLKAADDGRCIDTPLSFSLAPSDQLQGVVLTKQDKQQKITYCTNMNEVFEAKLGSADLLLNWDHLRGRIRDRVDAGDIGSAFLKLALDVAHVLPDGVDDQLARAAFHFQSAKGAKSKHADSVQAGLRVLSIDLGVRSFATCSVFELKDTAPTTGVAFPLAEFRLWAVHERSFTLELPGENVGAAGQQWRAQADAELRQLRGGLNRHRQLLRAATVQKGERDAYLTDLREAWSAKELWPFEASLLSELERCSTVADPLWQDTCKRAARLYRTEFGAVVSEWRSRTRSREDRKYAGKSMWSVQHLTDVRRFLQSWSLAGRASGDIRRLDRERGGVFAKDLLDHIDALKDDRLKTGADLIVQAARGFQRNEFGYWVQKHAPCHVILFEDLSRYRMRTDRPRRENSQLMQWAHRGVPDMVGMQGEIYGIQDRRDPDSARKHARQPLAAFCLDTPAAFSSRYHASTMTPGIRCHPLRKREFEDQGFLELLKRENEGLDLNGYKPGDLVPLPGGEVFVCLNANGLSRIHADINAAQNLQRRFWTQHGDAFRLPCGKSAVQGQIRWAPLSMGKRQAGALGGFGYLEPTGHDSGSCQWRKTTEAEWRRLSGAQKDRDEAAAAEDEELQGLEEELLERSGERVVFFRDPSGVVLPTDLWFPSAAFWSIVRAKTVGRLRSHLDAQAEASYAVAAGL

SEQ ID NO: 191 - protein, 1388 aa, unidentified
MSLNRIYQGRVAAVETGTALAKGNVEWMPAAGGDEVLWQHHELFQAAINYYLVALLALADKNNPVLGPLISQMDNPQSPYHVWGSFRRQGRQRTGLSQAVAPYITPGNNAPTLDEVFRSILAGNPTDRATLDAALMQLLKACDGAGAIQQEGRSYWPKFCDPDSTANFAGDPAMLRREQHRLLLPQVLHDPAITHDSPALGSFDTYSIATPDTRTPQLTGPKARARLEQAITLWRVRLPESAADFDRLASSLKKIPDDDSRLNLQGYVGSSAKGEVQARLFALLLFRHLERSSFTLGLLRSATPPPKNAETPPPAGVPLPAASAADPVRIARGKRSFVFRAFTSLPCWHGGDNIHPTWKSFDIAAFKYALTVINQIEEKTKERQKECAELETDFDYMHGRLAKIPVKYTTGEAEPPPILANDLRIPLLRELLQNIKVDTALTDGEAVSYGLQRRTIRGFRELRRIWRGHAPAGTVFSSELKEKLAGELRQFQTDNSTTIGSVQLFNELIQNPKYWPIWQAPDVETARQWADAGFADDPLAALVQEAELQEDIDALKAPVKLTPADPEYSRRQYDFNAVSKFGAGSRSANRHEPGQTERGHNTFTTEIAARNAADGNRWRATHVRIHYSAPRLLRDGLRRPDTDGNEALEAVPWLQPMMEALAPLPTLPQDLTGMPVFLMPDVTLSGERRILLNLPVTLEPAALVEQLGNAGRWQNQFFGSREDPFALRWPADGAVKTAKGKTHIPWHQDRDHFTVLGVDLGTRDAGALALLNVTAQKPAKPVHRIIGEADGRTWYASLADARMIRLPGEDARLFVRGKLVQEPYGERGRNASLLEWEDARNIILRLGQNPDELLGADPRRHSYPEINDKLLVALRRAQARLARLQNRSWRLRDLAESDKALDEIHAERAGEKPSPLPPLARDDAIKSTDEALLSQRDIIRRSFVQIANLILPLRGRRWEWRPHVEVPDCHILAQSDPGTDDTKRLVAGQRGISHERIEQIEELRRRCQSLNRALRHKPGERPVLGRPAKGEEIADPCPALLEKINRLRDQRVDQTAHAILAAALGVRLRAPSKDRAERRHRDIHGEYERFRAPADFVVIENLSRYLSSQDRARSENTRLMQWCHRQIVQKLRQLCETYGIPVLAVPAAYSSRFSSRDGSAGFRAVHLTPDHRHRMPWSRILARLKAHEEDGKRLEKTVLDEARAVRGLFDRLDRFNAGHVPGKPWRTLLAPLPGGPVFVPLGDATPMQADLNAAINIALRGIAAPDRHDIHHRLRAENKKRILSLRLGTQREKARWPGGAPAVTLSTPNNGASPEDSDALPERVSNLFVDIAGVANFERVTIEGVSQKFATGRGLWASVKQRAWNRVARLNETVTDNNRNEEEDDIPM

SEQ ID NO: 192 - protein, 1465 aa, Thermomonas hydrothermalis
MSEKTTQRAYTLRLNRASGECAVCQNNSCDCWHDALWATHKAVNRGAKAFGDWLLTLRGGLCHTLVEMEVPAKGNNPPQRPTDQERRDRRVLLALSWLSVEDEHGAPKEFIVATGRDSADDRAKKVEEKLREILEKRDFQEHEIDAWLQDCGPSLKAHIREDAVWVNRRALFDAAVERIKTLTWEEAWDFLEPFFGTQYFAGIGDGKDKDDAEGPARQGEKAKDLVQKAGQWLSARFGIGTGADFMSMAEAYEKIAKWASQAQNGDNGKATIEKLACALRPSEPPTLDTVLKCISGPGHKSATREYLKTLDKKSTVTQEDLNQLRKLADEDARNCRKKVGKKGKKPWADEVLKDVENSCELTYLQDNSPARHREFSVMLDHAARRVSMAHSWIKKAEQRRRQFESDAQKLKNLQERAPSAVEWLDRFCESRSMTTGANTGSGYRIRKRAIEGWSYVVQAWAEASCDTEDKRIAAARKVQADPEIEKFGDIQLFEALAADEAICVWRDQEGTQNPSILIDYVTGKTAEHNQKRFKVPAYRHPDELRHPVFCDFGNSRWSIQFAIHKEIRDRDKGAKQDTRQLQNRHGLKMRLWNGRSMTDVNLHWSSKRLTADLALDQNPNPNPTEVTRADRLGRAASSAFDHVKIKNVFNEKEWNGRLQAPRAELDRIAKLEEQGKTEQAEKLRKRLRWYVSFSPCLSPSGPFIVYAGQHNIQPKRSGQYAPHAQANKGRARLAQLILSRLPDLRILSVDLGHRFAAACAVWETLSSDAFRREIQGLNVLAGGSGEGDLFLHVEMTGDDGKRRTVVYRRIGPDQLLDNTPHPAPWARLDRQFLIKLQGEDEGVREASNEELWTVHKLEVEVGRTVPLIDRMVRSGFGKTEKQKERLKKLRELGWISAMPNEPSAETDEKEGEIRSISRSVDELMSSALGTLRLALKRHGNRARIAFAMTADYKPMPGGQKYYFHEAKEASKNDDETKRRDNQIEFLQDALSLWHDLFSSPDWEDNEAKKLWQNHIATLPNYQTPEEISAELKRVERNKKRKENRDKLRTAAKALAENDQLRQHLHDTWKERWESDDQQWKERLRSLKDWIFPRGKAEDNPSIRHVGGLSITRINTISGLYQILKAFKMRPEPDDLRKNIPQKGDDELENFNRRLLEARDRLREQRVKQLASRIIEAALGVGRIKIPKNGKLPKRPRTTVDTPCHAVVIESLKTYRPDDLRTRRENRQLMQWSSAKVRKYLKEGCELYGLHFLEVPANYTSRQCSRTGLPGIRCDDVPTGDFLKAPWWRRAINTAREKNGGDAKDRFLVDLYDHLNNLQSKGEALPATVRVPRQGGNLFIAGAQLDDTNKERRAIQADLNAAANIGLRALLDPDWRGRWWYVPCKDGTSEPALDRIEGSTAFNDVRSLPTGDNSSRRAPREIENLWRDPSGDSLESGTWSPTRAYWDTVQSRVIELLRRHAGLPTS

SEQ ID NO: 193 - protein, 370 aa, Methylobacterium nodulans
MYEAIVLADDANAQLANAFLGPLTDPNSAGFLEAFNKVDRPAPSWLDQVPASDPIDPAVLAEANAWLDTDAGRAWLVDTGAPPRWRSLAAKQDPIWPREFARKLGELRKEAASGTSAIIKALKRDFGVLPLFQPSLAPRILGSRSSLTPWDRLAFRLAVGHLLSWESWCTRARDEHTARVQRLEQFSSAHLKGDLATKVSTLREYERARKEQIAQLGLPMGERDFLITVRMTRGWDDLREKWRRSGDKGQEALHAIIATEQTRKRGRFGDPDLFRWLARPENHHVWADGHADAVGVLARVNAMERLVERSRDTALMTLPDPVAHPRSAQWEAEGGSNLRNYQLEAVGGELQITLPLLKAADDGRCIDTPL

SEQ ID NO: 194 - protein, 1501 aa, unidentified
MPQQAKPPVTQRAYTLRLRGADSNDPSWRDALWQTHEAVNRGAQAFGDWLLTLRGGLDHTLADTPVKGGKGKPDPDPTDEERKARRILLALSWLSVESKLGAPAGLIIAFGTEAAEERNRKVVAALEEILKSRGVDQNEINAWKKDCSASLSAAIRDDAVWVNRSKAFDEAVESIGSSGSSGSSLTREEPWDMLERFFGSRDAYLAPAKGSEDESSEAKQEDQAKDLVQKAGQWLSSRFGTGKGADFRRMATVYEAIAKWDGKASLEMAGDKAIADLATALSEFNPASNDLQGVLGLISGPGYKSATRNFLNQLAAQTTVTQQDFVSLKDKANNDAQECKQNTGSKGQRPYSNSILEKVESVCGFTYLQDGGPARHSEFAVILDHAARRVSLAHTWIKLAEAERRKFEEDAKKIDQVPEAAKDWLDRFCLERSGVSGALEPYRIRRRAVDGWKEVVAEWSKSDCKTVEDRIAAARALQDDPEIDKFGDIQLFEALAEDDAVCVWHKDGDAAKAPDPQPLIDYALAAEAEFKKRHFKVPAYRHPDALLHPIFCDFGKSRWDICFDVHKNMQTPFPRALCLTLWTGSEMKRIPLCWQSKRLARDLALGNNTGDAGASEVTRADRLGRAASRAASNVTKSDVVNIAGLFEQADWNGRLQAPRQQLEAIARYVEKHDWDQKAEKMRNAIQWLVTFSARLQPQGPWCAYAKIHGLKEDPQYWPHADTNKNRKGHARLILSRLPGLRVLAVDLGHRYAAACAVWEALSTEAFQREIKGRTILRGRTDGNALYCHTRHKANGKERVTIYRRIGADTLPDGKPHPAPWARLDRQFLIKLQGEEEGVREASNEEIWAVHQLEAALGRPVSLIDRLVASGWGGSDKQKARLEGLKQLGWDPADKPSLSVDELMSSAVRTMRLALKRHGDRARIAHYLITDEKTTPGGIKETLDEKGRIDLLQDALVLWHDLFSSRGWRDDTAKQLWNAHVAKLHGYKAPEEPGEDSSGAERKKKQRENREKLYDVAKALAQDVTLREALHDAWKKRWENDDERWKKQLRWFKDWVFPRGNHASDPTIRKRQLINPSGGNGRRGNHASDPTIRKRQLINPSGGNGRRGNHASDPTIRKVGGLSLPRLATLTEFRRKVQVGFFTRLKPDGTRAETKEQFGQSALDALEHLREQRVKQLASRIAEAALGVGRVRRPVEGKDPKRPDVRVDEPCHAIVIEDLTHYRPEETRTRRENRQLMTWSSSKVKKYLAEACQLHGLHLREVSASYTSRQDSRTGAPGVRCQDVPVKEFMRSPFWRKQVKQAEAKQAANKGDARERLLCDLNARWKDRTAADWEKAGAVRIPLQGGEIFVSADANSPAAKGIQADLNAAANIGLRALTDPDWAGKWWYVPCDPASFRPVRDKVDGSAVVNPDQPLRQSAQAQSGDAAKDKNGNKGAGKSKEVVNLWRDISSSPLECIEFGEWKEYAAYQNEVQCRVIRILKEQIKGRDKQPHEGSKEDDIPL

SEQ ID NO: 195 - protein, 1149 aa, Desulfovibrio inopinatus
MPTRTINLKLVLGKNPENATLRRALFSTHRLVNQATKRIEEFLLLCRGEAYRTVDNEGKEAEIPRHAVQEEALAFAKAAQRHNGCISTYEDQEILDVLRQLYERLVPSVNENNEAGDAQAANAWVSPLMSAESEGGLSVYDKVLDPPPVWMKLKEEKAPGWEAASQIWIQSDEGQSLLNKPGSPPRWIRKLRSGQPWQDDFVSDQKKKQDELTKGNAPLIKQLKEMGLLPLVNPFFRHLLDPEGKGVSPWDRLAVRAAVAHFISWESWNHRTRAEYNSLKLRRDEFEAASDEFKDDFTLLRQYEAKRHSTLKSIALADDSNPYRIGVRSLRAWNRVREEWIDKGATEEQRVTILSKLQTQLRGKFGDPDLFNWLAQDRHVHLWSPRDSVTPLVRINAVDKVLRRRKPYALMTFAHPRFHPRWILYEAPGGSNLRQYALDCTENALHITLPLLVDDAHGTWIEKKIRVPLAPSGQIQDLTLEKLEKKKNRLYYRSGFQQFAGLAGGAEVLFHRPYMEHDERSEESLLERPGAVWFKLTLDVATQAPPNWLDGKGRVRTPPEVHHFKTALSNKSKHTRTLQPGLRVLSVDLGMRTFASCSVFELIEGKPETGRAFPVADERSMDSPNKLWAKHERSFKLTLPGETPSRKEEEERSIARAEIYALKRDIQRLKSLLRLGEEDNDNRRDALLEQFFKGWGEEDVVPGQAFPRSLFQGLGAAPFRSTPELWRQHCQTYYDKAEACLAKHISDWRKRTRPRPTSREMWYKTRSYHGGKSIWMLEYLDAVRKLLLSWSLRGRTYGAINRQDTARFGSLASRLLHHINSLKEDRIKTGADSIVQAARGYIPLPHGKGWEQRYEPCQLILFEDLARYRFRVDRPRRENSQLMQWNHRAIVAETTMQAELYGQIVENTAAGFSSRFHAATGAPGVRCRFLLERDFDNDLPKPYLLRELSWMLGNTKVESEEEKLRLLSEKIRPGSLVPWDGGEQFATLHPKRQTLCVIHADMNAAQNLQRRFFGRCGEAFRLVCQPHGDDVLRLASTPGARLLGALQQLENGQGAFELVRDMGSTSQMNRFVMKSLGKKKIKPLQDNNGDDELEDVLSVLPEEDDTGRITVFRDSSGIFFPCNVWIPAKQFWPAVRAMIWKVMASHSLG

SEQ ID NO: 196 - protein, 1132 aa, Tuberibacillus calidus
MATKSFILKMKTKNNPQLRLSLWKTHELFNFGVAYYMDLLSLFRQKDLYMHNDEDPDHPVVLKKEEIQERLWMKVRETQQKNGFHGEVSKDEVLETLRALYEELVPSAVGKSGEANQISNKYLYPLTDPASQSGKGTANSGRKPRWKKLKEAGDPSWKDAYEKWEKERQEDPKLKILAALQSFGLIPLFRPFTENDHKAVISVKWMPKSKNQSVRKFDKDMFNQAIERFLSWESWNEKVAEDYEKTVSIYESLQKELKGISTKAFEIMERVEKAYEAHLREITFSNSTYRIGNRAIRGWTEIVKKWMKLDPSAPQGNYLDVVKDYQRRHPRESGDFKLFELLSRPENQAAWREYPEFLPLYVKYRHAEQRMKTAKKQATFTLCDPIRHPLWVRYEERSGTNLNKYRLIMNEKEKVVQFDRLICLNADGHYEEQEDVTVPLAPSQQFDDQIKFSSEDTGKGKHNFSYYHKGINYELKGTLGGARIQFDREHLLRRQGVKAGNVGRIFLNVTLNIEPMQPFSRSGNLQTSVGKALKVYVDGYPKVVNFKPKELTEHIKESEKNTLTLGVESLPTGLRVMSVDLGQRQAAAISIFEVVSEKPDDNKLFYPVKDTDLFAVHRTSFNIKLPGEKRTERRMLEQQKRDQAIRDLSRKLKFLKNVLNMQKLEKTDEREKRVNRWIKDREREEENPVYVQEFEMISKVLYSPHSVWVDQLKSIHRKLEEQLGKEISKWRQSISQGRQGVYGISLKNIEDIEKTRRLLFRWSMRPENPGEVKQLQPGERFAIDQQNHLNHLKDDRIKKLANQIVMTALGYRYDGKRKKWIAKHPACQLVLFEDLSRYAFYDERSRLENRNLMRWSRREIPKQVAQIGGLYGLLVGEVGAQYSSRFHAKSGAPGIRCRVVKEHELYITEGGQKVRNQKFLDSLVENNIIEPDDARRLEPGDLIRDQGGDKFATLDERGELVITHADINAAQNLQKRFWTRTHGLYRIRCESREIKDAVVLVPSDKDQKEKMENLFGIGYLQPFKQENDVYKWVKGEKIKGKKTSSQSDDKELVSEILQEASVMADELKGNRKTLFRDPSGYVFPKDRWYTGGRYFGTLEHLLKRKLAERRLFDGGSSRRGLFNGTDSNTNVE

SEQ ID NO: 197 - protein, 1108 aa, Bacillus thermoamylovorans
MATRSFILKIEPNEEVKKGLWKTHEVLNHGIAYYMNILKLIRQEAIYEHHEQDPKNPKKVSKAEIQAELWDFVLKMQKCNSFTHEVDKDVVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPFTDSNEPIVKEIKWMEKSRNQSVRRLDKDMFIQALERFLSWESWNLKVKEEYEKVEKEHKTLEERIKEDIQAFKSLEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKFVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYEERSRFENSKLMKWSRREIPRQVALQGEIYGLQVGEVGAQFSSRFHAKTGSPGIRCSVVTKEKLQDNRFFKNLQREGRLTLDKIAVLKEGDLYPDKGGEKFISLSKDRKLVTTHADINAAQNLQKRFWTRTHGFYKVYCKAYQVDGQTVYIPESKDQKQKIIEEFGEGYFILKDGVYEWGNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 198 - protein, 1108 aa, Bacillus sp.
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 199 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 200 - protein, 1115 aa, Alicyclobacillus hesperidum
MTVRSIRVKLAVGSPQYRDVRRGLWKTHEIMNQGVRYYCEWLVLMRQEPIYDEDEHGLTVVQRTREDIQAELLSRLRTLQSAHQHSGDMGTDEELLSLMRQLYEQLVPSSVDKNKSGDARMIARNFFNPLTNPNSQGGLGISNAGRKPKWLLKKLSGDPTWEEDYKKAMEQKQESSVSFLLLELRRFGLHPIFLPYTDTVLEVSWAPKKARQWVRKWDYDLFQQSIERMLSWESWTRRVKERFEKLVESEKKFYDENFATDPEFIKLAETLEGELQASSQGFVAVDEHAFQIRPRSMRGFDRVADEWCKLADDAPIEEYEAAIKRVQARLGRNFGSYVLFAHLAKPEYWSLWRSDPTKILRFARLRALQRAVARAKRHARLTLPDAIHHPIWIRYDAKGKNIYSYRLLIPEKRSKRYYVEFSSLIMPDGENRWAEHRNIRVPLAFSRQWERLHFSIMEDGSLCVQYRDPGVDEPLRAELGGAKIQFDRRYLIRRSSTLSAGECGPVYLNVSVDVNPAHRPDVQVLQSAKLVSVSRDTNRIYLRPENLSAYWKSQGDGTLPLRVMSVDLGVRSSAAVVICRLEHRDSVVSSGRRTATIYRIAGTDEFVAVQERAFLLRLPGEGKGTNEDAPLRDVYAQLGTIRQGIQILRSLLRLCDTKTPDERQEALHGLAQSLEPSGAWKDELHPHLVMLQGVVHDSVDNWKQKVISVHRQMERILGHAVREWKVARKNAGKPPIRRGAGGLSLRRIRQLEQERRTLVAWSNHAREPGQVVRIKRGTQVAQWLVERVNHLKEDRLKKLADLLIMTALGYVYDETKPSGHKWDKRYPPCQIILMEDLSRYRFQSDRPPSENSQLMAWSHRRLLEILKLQADLHKLIVGTVFPAFSSRFDAQSGAPGVRCRSVKKQDIENAAQGKGWLARELQRLNWTLEWLQPNDLIPTGDGELFVTPACCDRQKGIKIVHADLNAAQNLQRRFWGGHAESLCRVTCDVVERDGRRYAVPRISNAFADSFYKVFGQGVFVSTDEEDVYRWMVGEKISSRGRSRGRTSDEEAEAETWIDEAREQQGKVIALFRDASGQIHGGDWLVAKVFWGWVERLVTARLLSRMSEREAAAHKE

SEQ ID NO: 201 - protein, 1129 aa, Alicyclobacillus acidiphilus
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 202 - protein, 1144 aa, Alicyclobacillus macrosporangiidus
MAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 203 - protein, 1027 aa, Sulfobacillus thermosulfidooxidans
RQSREDASPQIIISASDLKADLLYHARQQQKEHVPRITGSDAEVLGALRQVYELIVPSSVGKSGDSKTIARKFLSPLTDPDSAGGRDQSASGRKPTWTKMKAEGNPLWEEKFRQWKDRKDNDPTPFVLNQLADYGLLPLIRLFTDVGENIFDPKKPGQFVRPWDRSMFQQAIERLMSWESWNQRVRQEWEALTQKHSAFYREQFTAEPDAALYRVAQSLEEEMRKEHQGFATDAPEAFRIRRVALKGFDRLLERWQKTLGKNGQSATLLDDIRRVQSDLGDKFGSAPLYQKLVDERWQRLWTVDPTFLQRYAAFNDLTQRLQRAKRVANLTLPDAVAHPIWSRYEGPNASSGNRYHIHLPTTGQPSSVTFDRILWPDGDGGWYERKRVTVFLRPSHQVDRIREAPTDSVVDNFPLVVEDQSARTILRASWGGAKLEYDRNRLPRQLKKGVPDSIYLSLTLNLDTTKPSGLFHMQQNGRVWIRKDVVMQYYNEIPGDNVQFKPLYVMSVDLGIRSAAAVSIFSVQLKTGIEEHRLTYPVADCPGLVAVHERSVLLTMPGERREQRDRRYEQQRQGLRELRTDMRGMNDLLRGAYVDGDRREEFLARLSKLEETSPELWEPVYRSLNDSKMAPAAEWERLVVYCHRQVEQSLSSRIQNLRSGRSAYRMSGGLSLDHVQDLERIRGIIASWTNHPRIPGSVVRWQQGRSHTVALGRHILELKRDRVKKVANYLIMTALGYAYDSKRARGEKWVRRYPSCHLMVFEDLTRYRFRTDRPRSENRQLMRWTHQELIAVTGIQAEPHGILVGTMYAGFSSRFDAVTKAPGVRGATVRQILRTRGMVRLKEIAADVGVDINTLRPHDVLPTGDGEYLLSVVRHRDSYRLKQVHADINAAHNLQRRLWTQDEVFRVSCRLALNSERVVATPPPSYNKRYGKGFFEKGDNGVYIWKTGGKIKISDMLEEDMDIPEDTAELLRGNSVTLFRDPSGTIAGGNWLEAKEFWGRVNSLVNKGVRDKILGGIPVDNSSAHAE

SEQ ID NO: 204 - protein, 1070 aa, Spirochaeta sp.
MGLLLPSLSRTVNVTIHLILHPRKKGSRHREYAVMLDHAVRKIFLAHNWIKRAEAERQKFEADLYKIDRVPQEARDWLDEFCRERTESTGSIDGYHIRRKAVLGWEALVEAWDQKDCLSVEDRIAAARDLQDNPGMDKFGDIWLYEALASAPCVWQKDGEPNAQILLDYVDAGEAEYKRSHYKVPAYRHPDPLLHPIFCDFGQSRWSISFDIHEFKKNGEKNPVNIHALTMGLVSKKRIVKTELKWSSKRLNSNLALSLESPEDAIEVSRATRLGRAAVGASQDRAVNIAGLFESAGWNGRLQAPRKQLEALAKLEEDKSAEALAKALRNRIKWFITFSPKLQPHGPWMEYAERFSGEAPSRAAVIKGKYTVIHQDKTRRRPLAKLHLCRMPGLRVLSVDLGHRHAAACAVWETLSSESMEKKCREAGCLPPAPEDLYLHLKKKNKTAVYRRIGGNFLPDGNEHPAPWAKLDRQFIIDLQGEEGCTRMALAGEIWQVHCMEKVFGRSIPLVDRLVRAGWGEKNKQPEILQELKQKGWVPLEVSKTNTGYHYSLCVDSLMTLAVNTVRFALRRHACRARIAYYMEGGAIPEGGLPENSGNKDFIVEALMLWYELATDSRWNGSWEANFWDENFDKKLAEIQDAVNEREGDKAKIIKQKERKELLKKEFIPLAEGLLENSRRISIASQWRMVWNEEDAIWQSELRSLRDWILPKGTRGKKRTIRHVGGLSLSRLAVIKSLYRVQKSFYTRMKPEGEPMDGTMAVGEGFGQKILDDLETMKEQRVKQLASRVVEAALGTGRIKKPENNKTPKRPFTAVDEPCHAVVIENLTHYRPENKRTRRENRQLMTWSSSKVKKYLFESCQLHGLYLFEVQASYTSRQDSRTGAPGVRCSELSVKKFLESPFRQREIAHAEENMAQENPCNRYLIALHNKWKNREYDKTAPPLRIPHWGGEIFVSALTGNTLQADLNAAANIGLQALLDPDWPGRWWYVPAVKGCDGRRIPHSKCSGAACLDNWRVGLKNNLYTGVRTPLPGKNKGSTSGEDVHKSNAVEKSTINLWRDISVLPLTEGQW

SEQ ID NO: 205 - protein, 1108 aa, synthetic construct
MATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYGERSRFENSRLMKWSRREIPRQVALQGEIYGLQVGEVGAQFSSRFHAKTGSPGIRCRVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSM

SEQ ID NO: 206 - protein, 1252 aa, unidentified
MTKLRHRQKKLTHDWAGSKKREVLGSNGKLQNPLLMPVKKGQVTEFRKAFSAYARATKGEMTDGRKNMFTHSFEPFKTKPSLHQCELADKAYQSLHSYLPGSLAHFLLSAHALGFRIFSKSGEATAFQASSKIEAYESKLASELACVDLSIQNLTISTLFNALTTSVRGKGEETSADPLIARFYTLLTGKPLSRDTQGPERDLAEVISRKIASSFGTWKEMTANPLQSLQFFEEELHALDANVSLSPAFDVLIKMNDLQGDLKNRTIVFDPDAPVFEYNAEDPADIIIKLTARYAKEAVIKNQNVGNYVKNAITTTNANGLGWLLNKGLSLLPVSTDDELLEFIGVERSHPSCHALIELIAQLEAPELFEKNVFSDTRSEVQGMIDSAVSNHIARLSSSRNSLSMDSEELERLIKSFQIHTPHCSLFIGAQSLSQQLESLPEALQSGVNSADILLGSTQYMLTNSLVEESIATYQRTLNRINYLSGVAGQINGAIKRKAIDGEKIHLPAAWSELISLPFIGQPVIDVESDLAHLKNQYQTLSNEFDTLISALQKNFDLNFNKALLNRTQHFEAMCRSTKKNALSKPEIVSYRDLLARLTSCLYRGSLVLRRAGIEVLKKHKIFESNSELREHVHERKHFVFVSPLDRKAKKLLRLTDSRPDLLHVIDEILQHDNLENKDRESLWLVRSGYLLAGLPDQLSSSFINLPIITQKGDRRLIDLIQYDQINRDAFVMLVTSAFKSNLSGLQYRANKQSFVVTRTLSPYLGSKLVYVPKDKDWLVPSQMFEGRFADILQSDYMVWKDAGRLCVIDTAKHLSNIKKSVFSSEEVLAFLRELPHRTFIQTEVRGLGVNVDGIAFNNGDIPSLKTFSNCVQVKVSRTNTSLVQTLNRWFEGGKVSPPSIQFERAYYKKDDQIHEDAAKRKIRFQMPATELVHASDDAGWTPSYLLGIDPGEYGMGLSLVSINNGEVLDSGFIHINSLINFASKKSNHQTKVVPRQQYKSPYANYLEQSKDSAAGDIAHILDRLIYKLNALPVFEALSGNSQSAADQVWTKVLSFYTWGDNDAQNSIRKQHWFGASHWDIKGMLRQPPTEKKPKPYIAFPGSQVSSYGNSQRCSCCGRNPIEQLREMAKDTSIKELKIRNSEIQLFDGTIKLFNPDPSTVIERRRHNLGPSRIPVADRTFKNISPSSLEFKELITIVSRSIRHSPEFIAKKRGIGSEYFCAYSDCNSSLNSEANAAANVAQKFQKQLFFEL

SEQ ID NO: 207 - protein, 1050 aa, unidentified
MRSNYHGGRNARQWRKQISGLARRTKETVFTYKFPLETDAAEIDFDKAVQTYGIAEGVGHGSLIGLVCAFHLSGFRLFSKAGEAMAFRNRSRYPTDAFAEKLSAIMGIQLPTLSPEGLDLIFQSPPRSRDGIAPVWSENEVRNRLYTNWTGRGPANKPDEHLLEIAGEIAKQVFPKFGGWDDLASDPDKALAAADKYFQSQGDFPSIASLPAAIMLSPANSTVDFEGDYIAIDPAAETLLHQAVSRCAARLGRERPDLDQNKGPFVSSLQDALVSSQNNGLSWLFGVGFQHWKEKSPKELIDEYKVPADQHGAVTQVKSFVDAIPLNPLFDTTHYGEFRASVAGKVRSWVANYWKRLLDLKSLLATTEFTLPESISDPKAVSLFSGLLVDPQGLKKVADSLPARLVSAEEAIDRLMGVGIPTAADIAQVERVADEIGAFIGQVQQFNNQVKQKLENLQDADDEEFLKGLKIELPSGDKEPPAINRISGGAPDAAAEISELEEKLQRLLDARSEHFQTISEWAEENAVTLDPIAAMVELERLRLAERGATGDPEEYALRLLLQRIGRLANRVSPVSAGSIRELLKPVFMEEREFNLFFHNRLGSLYRSPYSTSRHQPFSIDVGKAKAIDWIAGLDQISSDIEKALSGAGEALGDQLRDWINLAGFAISQRLRGLPDTVPNALAQVRCPDDVRIPPLLAMLLEEDDIARDVCLKAFNLYVSAINGCLFGALREGFIVRTRFQRIGTDQIHYVPKDKAWEYPDRLNTAKGPINAAVSSDWIEKDGAVIKPVETVRNLSSTGFAGAGVSEYLVQAPHDWYTPLDLRDVAHLVTGLPVEKNITKLKRLTNRTAFRMVGASSFKTHLDSVLLSDKIKLGDFTIIIDQHYRQSVTYGGKVKISYEPERLQVEAAVPVVDTRDRTVPEPDTLFDHIVAIDLGERSVGFAVFDIKSCLRTGEVKPIHDNNGNPVVGTVAVPSIRRLMKAVRSHRRRRQPNQKVNQTYSTALQNYRENVIGDVCNRIDTLMERYNAFPVLEFQIKNFQAGAKQLEIVYGS

SEQ ID NO: 208 - protein, 1273 aa, unidentified
MKKFELKQNFRNNYSGKTLRNFRQTLAQIANKKSSDSILTIKFKLDCSKTGKLPKYENLISLYDTIEDIKKGTLSYYLFTLIVSGFKFFGSASQAKAFSTKDIFKDNDFYNQFKIQSHLDLPDFVPSKIYQRLKKNVRSTNGKDNAFKASVIVAEYRKEIGKLKNKDESSEHQCEELFKKIGTALETRFSSWQDLINNCSTGCEIIDEILNDSFGTLPSIKKMVLASTTQSSDGEQDGIAIAYDPDSTFIKSDELLNPYFAVATILKSMPPEIQQDKKSAYVKANLTTPTHNALSWIFGKGLTLFQTESTEKLCAMFNVSDKRVIEQVQDAAKAVKLPAELDLNHCTLKFQDFRSSLGGHLDSWTTNYLKRLDELNDLLLNLPKNLSLPDIFMIDGKDFIEYSGCNRDEIQQMIDFVVNEQNRIKLQESLNALLGKGNNQICSDDISTVKDFSEIVNSLHSFVQQIDNSLEQSSNEANSIFSELKKKIEKNEKWDIWKNNLKKIPKLNKLSGGVPDAWKEIREIEQKFHEISENQKKHFTEVMEWIDAGNGTIDIFESRFKYDELLKKSKKNNLQSADELAFRSVLNKLGRFARQGNDLVCEKIKNWFKEQNIFDSSKDFNRYFINQKGFIFKHPSSKKDNSPYNLSANLLEKRYEVTNTVGALLEQCESDPAIVNDPFSMRSLVEFRALWFSINISGISKEQHIPTKIAQPKLDDSTYQESVSPTLKYRLEKEQITSSELNSIFTVYKSLLSGLSIRLSRNSFYLRTKFSWIGNNSLIYCPKETTWKIPAAYFKSDLWNEYKDKQILIVNEEYDVDVVKTFESVYKIVKSKDNNEKNRILPLLKQLPHDWMFKLPFGASNAEKCKVLKLEKNNKKFKPLSVSKDSLARLSGPSTYFNQIDEIMMNDESELSEMTLLADEPVRQQMSNGKIEIIPDDYVMSLAIPITRSLKKGNTESFPFKNIVSIDQGEAGFAYAVFKLSDCGNERAEPIATGLIPIPSIRRLIHSVKKYRGKKQRIQNFNQKFDSTMFTLRENVTGDICGLIVALMKKYNAFPILEKQVGNLESGSKQLMLVYKAVNSKFLAAKVDMQNDQRRSWWYQGNSWNTPILRISNPNQSNNKNIVKNINGKKYEELKIYPGYSVSAYMTSCICHVCGRNALELLKNDDSTGKVKKYQINQDGEVTIGGEVIKLYRKPDRLTPVKNLAKKGNRERTYASINERAPMSKDTTQSRYFCVFKNCPCHNKEQHADVNAAINIGRRFLKDCILDDNKEKD

SEQ ID NO: 209 - protein, 1258 aa, unidentified
MNARDWRKHVGVLAQQHKETTRTYTFPLDTTGSAIDFDAALQAYNAVEGVGYGSLLGLACAVHLSGFRLFSTGKEAATFRNRARYPNAAFQAALRKELGTTITTLTPETLDRLFSSRPKRRNGVPLPWNQDSIRDRLYTNWVKPRPGDTPDAVLFQIATGIAQEITEDVSSWTDLAKNSDRGLKAAHRYFARVGGFPAFDNLTPPATVQPTDTTIDYDPNAPFHLVSHADQTLIHQSISLCAHRIRQEDPALDPNKSGFIKQLQNNFLSQTFYGLSWLFGAGYVHFRECTANDLAIQYGIPNNCRDGIHQIKSFADAILPNTFFEKKHYRKDSRSVGKKAKSWISNYWQRLLQLQTWVDDHTWVTLPQELTEAQFKPLFRGLLVDAVELMAIAERLPQRLADCRDSLDCLMGKGPQAATKNDVEIVEKVREEIESFVGQIEQLGNQLRHQLENENNDQVHRDNLHQLKNRLPLDLRRPQALNKISGGVPDVAKSIRGLETQLDQVLKERRSHFGRLTKWAKECGITLDPLQPLIESEKQRVAERGSAHDAKELAIRLLLQRIGRLGHRLSPTNATAIQELLRPVFAVKREFNLFFHNHMGALYRSPYSTSRHQPFQINVDVAHGTDWIGTIETLIQNLFTQIQDDALLRDLVQLEGFVFSHKLRALPGVIPSELARPNNLQQMGLPALLLVLLQADQVHRETVLRVFNLYGSAINGYLFQALRPGFIVRAGFQRLETKKLRYVPKAQSWQYPDRLHHAKSAIKNSLSAGWIKKNHQGAILPQKTLTALVKQKSLKDTGVPEYLVQAPHDWYVPIDLRGPAIPIEGLTVGTEGPELTQLGPMKDDCAFRAIGPSSFKSKIDAGLLPQDVKYGDMTLIFDQHYQQSISFANGTFSIQYQPTSLQVKAAIPVVDKRPRDTRNNSHLYDRIVAIDLGERKIGYAIFDLKQVLKSEQLEPMREDGKPLIGSISIRSIRGLMKAVQTHRNRRQPNYRIDQTYSKALMHYRESVIGDVCNAIDTLCARYGGFPVLESSVRNFEVGSAQLKTVYGSVSRRYTWSAVDAHKNQRQQYWLGGTKDKIPIWTHPYLMTREWDEKNSKWSNRSKPLKMHPGVEVHPAGTSQICHQCKRNPIGALWNVADTVVLDDQGQLDLDDGTIRLNSGYIDTTEIKRARRKKIRLPENKPLTGSHKTSHVRAVARRNLRQPPKSTRAKDTTQSRYTCLYVDCGHECHADENAAINIGRKYLQERIHIEASRQALSTR

SEQ ID NO: 210 - protein, 1269 aa, unidentified
MVAGLKKIKRDGVTMKSNYHGGVKARAWRKRIGGLARRQKETVFTYKFPLETEEAGIDFDKAVQTYGIAEGISQGSLIGLVCAFHLSGFRLFSKADETKAFCNQGRYPNQAFAEKLRNELSVTLPKLSPQSLDVLFQSSPKSKNGVAPEWSKNAIRNRLYTNWTGKGAGTNPDEHLLEIAEDIAAEIDSDLDGWKDLEEHPEKGLSAADRYFQAQGDFPSLTGLPPSVPLTPQNSTVAFEGDPVCLNPSDNTLLHQAVARCAGRILQEQPNLSPDKNRFINQLQDELVSSQNNGLSWLFGVGFKYWKEMSVDQLADDYKVKSTDLDALKQVKSFIDAIPLNPLFDTPHYGEFRASVAGKMRSWVKNYWKRLLDLKSQLGTANINLPEGLDEQRAENLFSGLLIDSKGLRQVTDKLPSRLKKAEDTIDRLMGDGNPTSDDIEQVETVAAEISAFIGQVEQFNNQLEQRLENPLEGDDETFLKQLKIDLPAEFKKPPAINRISGGSPDPTAEIAELEEKLDRLMSARKEHYETIAEWASANKVTLDPMEAMTTLEAQRLTERGAEGDQEEFALRLLLQRIGRLANRLSPQGATAIRDLLRPVFTEKREFNLFFHNRMGSLYRSPYSTSRHQPFTIDVAVAKNTDWMDALDGIAETIMKGLSQAGDELSLRQLEEDEVSREVCLKAFNLYVSAINGCLFRALREGFIVRTKFQRLERDVLSYVPKTKLWNYPQRLDTARGPIHSALAAAWINKEGSVIDPVETVTALSDTGFSDDGIPEYLVQAPHDWYLRDWINISGFSLSQRLRGLPDTVPGELALVRSADDVRIPPMLALTPIDLRDISKPVSGLPVKKNITGLKRQKKQTAFRMVGPSSFKSHLDSTLLSEEVKLGDFTLIFDQYYKQRVSYNGRVKITFEPDRLHVEAAVPVIDKRVRPSTEEDALFDHLLAIDLGEKRVGYAVYDIKACLRTGDIKPLEDGDGKPIVGSVAVPSIRRLMKAVRSHRQQRQPNQKVNQTYSTALMNYRENVIGDVCNRIDTLMEKYNAFPVLESSVMNFEAGSRQLEMVYGSVLHRYTYSKIDAHTAKRKEYWYTGEYWDHPYLMAHKWNERTRSYSGSLSALTLYPGVMVHPAGTSQRCHQCKRNPMVEIKQLTGQVEINADGSLELDDGTICLYEGYDYSPEEYKKAKREKRRLDPNVPLSGRHQAKHVSAVAKRNLRRPTVSMMSGDTTQARYVCLYTDCDFTGHADENAAINIGWKYLTERIALSESKDKAGV

SEQ ID NO: 211 - protein, 1210 aa, unidentified
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFERFRKLKNIKVLGQMKKI

SEQ ID NO: 212 - DNA, 184 bp, synthetic construct
  source 1..184: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaa

SEQ ID NO: 213 - DNA, 194 bp, synthetic construct
  source 1..194: mol_type = other DNA
gctattctcgcagctcaccagtttgagagctatgctggaaacagcatagcaagttcaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaa

SEQ ID NO: 214 - DNA, 179 bp, synthetic construct
  source 1..179: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggcacaattaacatctcaatcaaggtaaatgcttgagctgcgagaa

SEQ ID NO: 215 - DNA, 179 bp, synthetic construct
  source 1..179: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggagcatttaccttgattgagatgttaattgtgtgagctgcgagaa

SEQ ID NO: 216 - DNA, 182 bp, synthetic construct
  source 1..182: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggcaggtttttgacgaaagtgatccagatgatccagtgagctgcgagaa

SEQ ID NO: 217 - DNA, 182 bp, synthetic construct
  source 1..182: mol_type = other DNA
gctattctcgcagctcaccagttttagagctagaaatagcaagttaaaataaggctagtccgttatcaacttgaaaaagtggcaccgagtcggtgcgagtcggtgcgacgagcgcggcgatatcatcatccatggctggatcatctggatcactttcgtcaaaaacctgtgagctgcgagaa

SEQ ID NO: 218 - DNA, 96 bp, synthetic construct
  source 1..96: mol_type = other DNA
gaagccggccttgcacatgcgttttagagctagaaatagcaagttaaaataaggctagtccgttatcaacttgaaaaagtggcaccgagtcggtgc

SEQ ID NO: 219 - DNA, 164 bp, synthetic construct
  source 1..164: mol_type = other DNA
gaagccggccttgcacatgcgttttagagctagaaatagcaagttaaaataaggctagtccgttatcaacttgaaaaagtggcaccgagtcggtgcatatcatcatccatggtaccgttcgtatagcatacattatacgaagttattgagctgcgagaatagcc

SEQ ID NO: 220 - DNA, 172 bp, synthetic construct
  source 1..172: mol_type = other DNA
gaagccggccttgcacatgcgttttagagctagaaatagcaagttaaaataaggctagtccgttatcaacttgaaaaagtggcaccgagtcggtgcgacgagcgcggcgatatcatcatccatggtaccgttcgtatagcatacattatacgaagttattgagctgcgagaa

SEQ ID NO: 221 - DNA, 189 bp, synthetic construct
  source 1..189: mol_type = other DNA
gctattctcgcagctcaccagttttagagctagaaatagcaagttaaaataaggctagtccgttatcaacttgaaaaagtggcaccgagtcggtgctcgacgacgagcgcggcgatatcatcatccatggccggatgatcctgacgacggagaccgccgtcgtcgacaagccggcctgagctgcgagaa

SEQ ID NO: 222 - DNA, 181 bp, synthetic construct
  source 1..181: mol_type = other DNA
gctattctcgcagctcaccagttttagagctagaaatagcaagttaaaataaggctagtccgttatcaacttgaaaaagtggcaccgagtcggtgcgagcgcggcgatatcatcatccatggccggatgatcctgacgacggagaccgccgtcgtcgacaagccggcctgagctgcgagaa

SEQ ID NO: 223 - DNA, 178 bp, synthetic construct
  source 1..178: mol_type = other DNA
gctattctcgcagctcaccagttttagagctagaaatagcaagttaaaataaggctagtccgttatcaacttgaaaaagtggcaccgagtcggtgccgcggcgatatcatcatccatggccggatgatcctgacgacggagaccgccgtcgtcgacaagccggcctgagctgcgagaa

SEQ ID NO: 224 - DNA, 175 bp, synthetic construct
  source 1..175: mol_type = other DNA
gctattctcgcagctcaccagttttagagctagaaatagcaagttaaaataaggctagtccgttatcaacttgaaaaagtggcaccgagtcggtgcggcgatatcatcatccatggccggatgatcctgacgacggagaccgccgtcgtcgacaagccggcctgagctgcgagaa

SEQ ID NO: 225 - DNA, 171 bp, synthetic construct
  source 1..171: mol_type = other DNA
gctattctcgcagctcaccagttttagagctagaaatagcaagttaaaataaggctagtccgttatcaacttgaaaaagtggcaccgagtcggtgcatatcatcatccatggccggatgatcctgacgacggagaccgccgtcgtcgacaagccggcctgagctgcgagaa

SEQ ID NO: 226 - DNA, 194 bp, synthetic construct
  source 1..194: mol_type = other DNA
gctattctcgcagctcaccagttttagagctagaaatagcaagttaaaataaggctagtccgttatcaacttgaaaaagtggcaccgagtcggtgctcgacgacgagcgcggcgatatcatcatccatggccggatgatcctgacgacggagaccgccgtcgtcgacaagccggcctgagctgcgagaatagcc

SEQ ID NO: 227 - DNA, 189 bp, synthetic construct
  source 1..189: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagaccgccgtcgtcgacaagccggcctgagctgcgagaatagcc

SEQ ID NO: 228 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcatatcatcatccatggccggatgatcctgacgacggagaccgccgtcgtcgacaagccggcctgagctgcgagaatagcc

SEQ ID NO: 229 - DNA, 194 bp, synthetic construct
  source 1..194: mol_type = other DNA
gctgtctccgccgcccgccagttttagagctagaaatagcaagttaaaataaggctagtccgttatcaacttgaaaaagtggcaccgagtcggtgcctgcccatccgcggcggcacgggggtcgcagtcgccatgccggatgatcctgacgacggagaccgccgtcgtcgacaagccggcccgggcggcggaga

SEQ ID NO: 230 - DNA, 189 bp, synthetic construct
  source 1..189: mol_type = other DNA
gctgtctccgccgcccgccagttttagagctagaaatagcaagttaaaataaggctagtccgttatcaacttgaaaaagtggcaccgagtcggtgccatccgcggcggcacgggggtcgcagtcgccatgccggatgatcctgacgacggagaccgccgtcgtcgacaagccggcccgggcggcggaga

SEQ ID NO: 231 - DNA, 184 bp, synthetic construct
  source 1..184: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagaccgccgtcgtcgacaagccggcccgggcggcggaga

SEQ ID NO: 232 - DNA, 179 bp, synthetic construct
  source 1..179: mol_type = other DNA
gctgtctccgccgcccgccagttttagagctagaaatagcaagttaaaataaggctagtccgttatcaacttgaaaaagtggcaccgagtcggtgcggcacgggggtcgcagtcgccatgccggatgatcctgacgacggagaccgccgtcgtcgacaagccggcccgggcggcggaga

SEQ ID NO: 233 - DNA, 174 bp, synthetic construct
  source 1..174: mol_type = other DNA
gctgtctccgccgcccgccagttttagagctagaaatagcaagttaaaataaggctagtccgttatcaacttgaaaaagtggcaccgagtcggtgcgggggtcgcagtcgccatgccggatgatcctgacgacggagaccgccgtcgtcgacaagccggcccgggcggcggaga

SEQ ID NO: 234 - DNA, 199 bp, synthetic construct
  source 1..199: mol_type = other DNA
gctgtctccgccgcccgccagttttagagctagaaatagcaagttaaaataaggctagtccgttatcaacttgaaaaagtggcaccgagtcggtgcctgcccatccgcggcggcacgggggtcgcagtcgccatgccggatgatcctgacgacggagaccgccgtcgtcgacaagccggcccgggcggcggagacagcg

SEQ ID NO: 235 - DNA, 194 bp, synthetic construct
  source 1..194: mol_type = other DNA
gctgtctccgccgcccgccagttttagagctagaaatagcaagttaaaataaggctagtccgttatcaacttgaaaaagtggcaccgagtcggtgccatccgcggcggcacgggggtcgcagtcgccatgccggatgatcctgacgacggagaccgccgtcgtcgacaagccggcccgggcggcggagacagcg

SEQ ID NO: 236 - DNA, 189 bp, synthetic construct
  source 1..189: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagaccgccgtcgtcgacaagccggcccgggcggcggagacagcg

SEQ ID NO: 237 - DNA, 184 bp, synthetic construct
  source 1..184: mol_type = other DNA
gctgtctccgccgcccgccagttttagagctagaaatagcaagttaaaataaggctagtccgttatcaacttgaaaaagtggcaccgagtcggtgcggcacgggggtcgcagtcgccatgccggatgatcctgacgacggagaccgccgtcgtcgacaagccggcccgggcggcggagacagcg

SEQ ID NO: 238 - DNA, 179 bp, synthetic construct
  source 1..179: mol_type = other DNA
gctgtctccgccgcccgccagttttagagctagaaatagcaagttaaaataaggctagtccgttatcaacttgaaaaagtggcaccgagtcggtgcgggggtcgcagtcgccatgccggatgatcctgacgacggagaccgccgtcgtcgacaagccggcccgggcggcggagacagcg

SEQ ID NO: 239 - DNA, 96 bp, synthetic construct
  source 1..96: mol_type = other DNA
gcgtggtggggccgccagcggttttagagctagaaatagcaagttaaaataaggctagtccgttatcaacttgaaaaagtggcaccgagtcggtgc

SEQ ID NO: 240 - DNA, 180 bp, synthetic construct
  source 1..180: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggatgatcctgacgacggagaccgccgtcgtcgacaagccggtgagctgcgagaa

SEQ ID NO: 241 - DNA, 178 bp, synthetic construct
  source 1..178: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggatgatcctgacgacggagaccgccgtcgtcgacaagccgtgagctgcgagaa

SEQ ID NO: 242 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatgatcctgacgacggagaccgccgtcgtcgacaagcctgagctgcgagaa

SEQ ID NO: 243 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgatcctgacgacggagaccgccgtcgtcgacaagctgagctgcgagaa

SEQ ID NO: 244 - DNA, 182 bp, synthetic construct
  source 1..182: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgcggatgatcctgacgacggagaccgccgtcgtcgacaagccggccgggcggcggaga

SEQ ID NO: 245 - DNA, 180 bp, synthetic construct
  source 1..180: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgggatgatcctgacgacggagaccgccgtcgtcgacaagccggcgggcggcggaga

SEQ ID NO: 246 - DNA, 178 bp, synthetic construct
  source 1..178: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatggatgatcctgacgacggagaccgccgtcgtcgacaagccgcgggcggcggaga

SEQ ID NO: 247 - DNA, 176 bp, synthetic construct
  source 1..176: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgatgatcctgacgacggagaccgccgtcgtcgacaagcccgggcggcggaga

SEQ ID NO: 248 - DNA, 189 bp, synthetic construct
  source 1..189: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagaccgccgtcgtcgacaagccggcctcctccaggcaatacgcg

SEQ ID NO: 249 - DNA, 184 bp, synthetic construct
  source 1..184: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagaccgccgtcgtcgacaagccggcctcctccaggcaat

SEQ ID NO: 250 - DNA, 182 bp, synthetic construct
  source 1..182: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgcccggatgatcctgacgacggagaccgccgtcgtcgacaagccggctcctccaggcaat

SEQ ID NO: 251 - DNA, 180 bp, synthetic construct
  source 1..180: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccggatgatcctgacgacggagaccgccgtcgtcgacaagccggtcctccaggcaat

SEQ ID NO: 252 - DNA, 178 bp, synthetic construct
  source 1..178: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccgatgatcctgacgacggagaccgccgtcgtcgacaagccgtcctccaggcaat

SEQ ID NO: 253 - DNA, 176 bp, synthetic construct
  source 1..176: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccatgatcctgacgacggagaccgccgtcgtcgacaagcctcctccaggcaat

SEQ ID NO: 254 - DNA, 97 bp, synthetic construct
  source 1..97: mol_type = other DNA
gagccgagcacgaggggatacgttttagagctagaaatagcaagttaaaataaggctagtccgttatcaacttgaaaaagtggcaccgagtcggtgc

SEQ ID NO: 255 - DNA, 167 bp, synthetic construct
  source 1..167: mol_type = other DNA
gctattctcgcagctcaccagttttagagctagaaatagcaagttaaaataaggctagtccgttatcaacttgaaaaagtggcaccgagtcggtgcggcgatatcatcatccatggatgatcctgacgacggagaccgccgtcgtcgacaagcctgagctgcgagaa

SEQ ID NO: 256 - DNA, 162 bp, synthetic construct
  source 1..162: mol_type = other DNA
gctattctcgcagctcaccagttttagagctagaaatagcaagttaaaataaggctagtccgttatcaacttgaaaaagtggcaccgagtcggtgctatcatcatccatggatgatcctgacgacggagaccgccgtcgtcgacaagcctgagctgcgagaa

SEQ ID NO: 257 - DNA, 157 bp, synthetic construct
  source 1..157: mol_type = other DNA
gctattctcgcagctcaccagttttagagctagaaatagcaagttaaaataaggctagtccgttatcaacttgaaaaagtggcaccgagtcggtgctcatccatggatgatcctgacgacggagaccgccgtcgtcgacaagcctgagctgcgagaa

SEQ ID NO: 258 - DNA, 163 bp, synthetic construct
  source 1..163: mol_type = other DNA
gctattctcgcagctcaccagttttagagctagaaatagcaagttaaaataaggctagtccgttatcaacttgaaaaagtggcaccgagtcggtgcggcgatatcatcatccatggatgatcctgacgacggagaccgccgtcgtcgacaagcctgagctgcg

SEQ ID NO: 259 - DNA, 158 bp, synthetic construct
  source 1..158: mol_type = other DNA
gctattctcgcagctcaccagttttagagctagaaatagcaagttaaaataaggctagtccgttatcaacttgaaaaagtggcaccgagtcggtgctatcatcatccatggatgatcctgacgacggagaccgccgtcgtcgacaagcctgagctgcg

SEQ ID NO: 260 - DNA, 153 bp, synthetic construct
  source 1..153: mol_type = other DNA
gctattctcgcagctcaccagttttagagctagaaatagcaagttaaaataaggctagtccgttatcaacttgaaaaagtggcaccgagtcggtgctcatccatggatgatcctgacgacggagaccgccgtcgtcgacaagcctgagctgcg

SEQ ID NO: 261 - DNA, 167 bp, synthetic construct
  source 1..167: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatgatcctgacgacggagaccgccgtcgtcgacaagcccgggcggcggaga

SEQ ID NO: 262 - DNA, 162 bp, synthetic construct
  source 1..162: mol_type = other DNA
gctgtctccgccgcccgccagttttagagctagaaatagcaagttaaaataaggctagtccgttatcaacttgaaaaagtggcaccgagtcggtgcgtcgcagtcgccatgatgatcctgacgacggagaccgccgtcgtcgacaagcccgggcggcggaga

SEQ ID NO: 263 - DNA, 157 bp, synthetic construct
  source 1..157: mol_type = other DNA
gctgtctccgccgcccgccagttttagagctagaaatagcaagttaaaataaggctagtccgttatcaacttgaaaaagtggcaccgagtcggtgcagtcgccatgatgatcctgacgacggagaccgccgtcgtcgacaagcccgggcggcggaga

SEQ ID NO: 264 - DNA, 163 bp, synthetic construct
  source 1..163: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatgatcctgacgacggagaccgccgtcgtcgacaagcccgggcggcg

SEQ ID NO: 265 - DNA, 158 bp, synthetic construct
  source 1..158: mol_type = other DNA
gctgtctccgccgcccgccagttttagagctagaaatagcaagttaaaataaggctagtccgttatcaacttgaaaaagtggcaccgagtcggtgcgtcgcagtcgccatgatgatcctgacgacggagaccgccgtcgtcgacaagcccgggcggcg

SEQ ID NO: 266 - DNA, 153 bp, synthetic construct
  source 1..153: mol_type = other DNA
gctgtctccgccgcccgccagttttagagctagaaatagcaagttaaaataaggctagtccgttatcaacttgaaaaagtggcaccgagtcggtgcagtcgccatgatgatcctgacgacggagaccgccgtcgtcgacaagcccgggcggcg

SEQ ID NO: 267 - DNA, 180 bp, synthetic construct
  source 1..180: mol_type = other DNA
gagaagcggcgtccggggctagttttagagctagaaatagcaagttaaaataaggctagtccgttatcaacttgaaaaagtggcaccgagtcggtgctctttgtccagagtcacagccataccggatgatcctgacgacggagaccgccgtcgtcgacaagccggccccccggacgccgc

SEQ ID NO: 268 - DNA, 179 bp, synthetic construct
  source 1..179: mol_type = other DNA
gggcacggggccatgtacaagttttagagctagaaatagcaagttaaaataaggctagtccgttatcaacttgaaaaagtggcaccgagtcggtgcggcgtcggcagcccgatcccgttgccggatgatcctgacgacggagaccgccgtcgtcgacaagccggcctacatggccccgt

SEQ ID NO: 269 - DNA, 185 bp, synthetic construct
  source 1..185: mol_type = other DNA
gtgtcaggtggggcggggctagttttagagctagaaatagcaagttaaaataaggctagtccgttatcaacttgaaaaagtggcaccgagtcggtgcgctggctcctcccctggcaccataccggatgatcctgacgacggagaccgccgtcgtcgacaagccggccccccgccccacctgacac

SEQ ID NO: 270 - DNA, 184 bp, synthetic construct
  source 1..184: mol_type = other DNA
gagtgggtcagacgagcaggagttttagagctagaaatagcaagttaaaataaggctagtccgttatcaacttgaaaaagtggcaccgagtcggtgccgactcctcccccatgcagccctccatcccggatgatcctgacgacggagaccgccgtcgtcgacaagccggcctgctcgtctgacc

SEQ ID NO: 271 - DNA, 97 bp, synthetic construct
  source 1..97: mol_type = other DNA
gcagccacccgctctcggcccgttttagagctagaaatagcaagttaaaataaggctagtccgttatcaacttgaaaaagtggcaccgagtcggtgc

SEQ ID NO: 272 - DNA, 97 bp, synthetic construct
  source 1..97: mol_type = other DNA
gtgtagtcaggccgctcacccgttttagagctagaaatagcaagttaaaataaggctagtccgttatcaacttgaaaaagtggcaccgagtcggtgc

SEQ ID NO: 273 - DNA, 97 bp, synthetic construct
  source 1..97: mol_type = other DNA
gctgacaagtctacggaacctgttttagagctagaaatagcaagttaaaataaggctagtccgttatcaacttgaaaaagtggcaccgagtcggtgc

SEQ ID NO: 274 - DNA, 96 bp, synthetic construct
  source 1..96: mol_type = other DNA
gctcctccagcgccttgaccgttttagagctagaaatagcaagttaaaataaggctagtccgttatcaacttgaaaaagtggcaccgagtcggtgc

SEQ ID NO: 275 - DNA, 96 bp, synthetic construct
  source 1..96: mol_type = other DNA
gctattctcgcagctcaccagttttagagctagaaatagcaagttaaaataaggctagtccgttatcaacttgaaaaagtggcaccgagtcggtgc

SEQ ID NO: 276 - DNA, 96 bp, synthetic construct
  source 1..96: mol_type = other DNA
agaagcggcgtccggggctagttttagagctagaaatagcaagttaaaataaggctagtccgttatcaacttgaaaaagtggcaccgagtcggtgc

SEQ ID NO: 277 - DNA, 96 bp, synthetic construct
  source 1..96: mol_type = other DNA
gggcacggggccatgtacaagttttagagctagaaatagcaagttaaaataaggctagtccgttatcaacttgaaaaagtggcaccgagtcggtgc

SEQ ID NO: 278 - DNA, 96 bp, synthetic construct
  source 1..96: mol_type = other DNA
gcgtattgcctggaggatgggttttagagctagaaatagcaagttaaaataaggctagtccgttatcaacttgaaaaagtggcaccgagtcggtgc

SEQ ID NO: 279 - DNA, 96 bp, synthetic construct
  source 1..96: mol_type = other DNA
tgtcaggtggggcggggctagttttagagctagaaatagcaagttaaaataaggctagtccgttatcaacttgaaaaagtggcaccgagtcggtgc

SEQ ID NO: 280 - DNA, 96 bp, synthetic construct
  source 1..96: mol_type = other DNA
agtgggtcagacgagcaggagttttagagctagaaatagcaagttaaaataaggctagtccgttatcaacttgaaaaagtggcaccgagtcggtgc

SEQ ID NO: 281 - DNA, 96 bp, synthetic construct
  source 1..96: mol_type = other DNA
gctgtctccgccgcccgccagttttagagctagaaatagcaagttaaaataaggctagtccgttatcaacttgaaaaagtggcaccgagtcggtgc

SEQ ID NO: 282 - DNA, 96 bp, synthetic construct
  source 1..96: mol_type = other DNA
ggggtcgcagtcgccatggcgttttagagctagaaatagcaagttaaaataaggctagtccgttatcaacttgaaaaagtggcaccgagtcggtgc

SEQ ID NO: 283 - DNA, 184 bp, synthetic construct
  source 1..184: mol_type = other DNA
  misc_difference 148..149: note = cg, gc, at, ta, gg, tt, ga, ag, cc, tc, ct, aa, tg, gt, ca, or ac
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagnncgccgtcgtcgacaagccggcctgagctgcgagaa

SEQ ID NO: 284 - DNA, 183 bp, synthetic construct
  source 1..183: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgccggatgatcctgacgacggagaccgccgtcgtcgacaagccggcctgagctgcgagaa

SEQ ID NO: 285 - DNA, 183 bp, synthetic construct
  source 1..183: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgccggatgatcctgacgacggagagcgccgtcgtcgacaagccggcctgagctgcgagaa

SEQ ID NO: 286 - DNA, 189 bp, synthetic construct
  source 1..189: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccccggatgatcctgacgacggagtccgccgtcgtcgacaagccggcctcctccaggcaatacgcg

SEQ ID NO: 287 - DNA, 189 bp, synthetic construct
  source 1..189: mol_type = other DNA
gctgtctccgccgcccgccagttttagagctagaaatagcaagttaaaataaggctagtccgttatcaacttgaaaaagtggcaccgagtcggtgcgcggcggcacgggggtcgcagtcgccatgccggatgatcctgacgacggagctcgccgtcgtcgacaagccggcccgggcggcggagacagcg

SEQ ID NO: 288 - DNA, 96 bp, synthetic construct
  source 1..96: mol_type = other DNA
gtcacctccaatgactaggggttttagagctagaaatagcaagttaaaataaggctagtccgttatcaacttgaaaaagtggcaccgagtcggtgc

SEQ ID NO: 289 - DNA, 96 bp, synthetic construct
  source 1..96: mol_type = other DNA
gggcaaccacaaacccacgagttttagagctagaaatagcaagttaaaataaggctagtccgttatcaacttgaaaaagtggcaccgagtcggtgc

SEQ ID NO: 290 - DNA, 194 bp, synthetic construct
  source 1..194: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggctatgccggatgatcctgacgacggagtccgccgtcgtcgacaagccggccctagctgagctgcgagaa

SEQ ID NO: 291 - DNA, 189 bp, synthetic construct
  source 1..189: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgccggatgatcctgacgacggagtccgccgtcgtcgacaagccggccctatgagctgcgagaa

SEQ ID NO: 292 - DNA, 184 bp, synthetic construct
  source 1..184: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggccggatgatcctgacgacggagtccgccgtcgtcgacaagccggcctgagctgcgagaa

SEQ ID NO: 293 - DNA, 179 bp, synthetic construct
  source 1..179: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggatgatcctgacgacggagtccgccgtcgtcgacaagccgtgagctgcgagaa

SEQ ID NO: 294 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgatcctgacgacggagtccgccgtcgtcgacaagctgagctgcgagaa

SEQ ID NO: 295 - DNA, 169 bp, synthetic construct
  source 1..169: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatcctgacgacggagtccgccgtcgtcgacatgagctgcgagaa

SEQ ID NO: 296 - DNA, 164 bp, synthetic construct
  source 1..164: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcctgacgacggagtccgccgtcgtcgtgagctgcgagaa

SEQ ID NO: 297 - DNA, 159 bp, synthetic construct
  source 1..159: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgacgacggagtccgccgtcgtgagctgcgagaa

SEQ ID NO: 298 - DNA, 154 bp, synthetic construct
  source 1..154: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggacgacggagtccgccgtgagctgcgagaa

SEQ ID NO: 299 - DNA, 149 bp, synthetic construct
  source 1..149: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggacggagtccgtgagctgcgagaa

SEQ ID NO: 300 - DNA, 144 bp, synthetic construct
  source 1..144: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcggagttgagctgcgagaa

SEQ ID NO: 301 - DNA, 182 bp, synthetic construct
  source 1..182: mol_type = other DNA
gaagccggccttgcacatgcgttttagagctagaaatagcaagttaaaataaggctagtccgttatcaacttgaaaaagtggcaccgagtcggtgctcgacgacgagcgcggcgatatcatcatccatggtaccgttcgtatagcatacattatacgaagttattgagctgcgagaatagcc

SEQ ID NO: 302 - DNA, 177 bp, synthetic construct
  source 1..177: mol_type = other DNA
gaagccggccttgcacatgcgttttagagctagaaatagcaagttaaaataaggctagtccgttatcaacttgaaaaagtggcaccgagtcggtgcgacgagcgcggcgatatcatcatccatggtaccgttcgtatagcatacattatacgaagttattgagctgcgagaatagcc

SEQ ID NO: 303 - DNA, 177 bp, synthetic construct
  source 1..177: mol_type = other DNA
gaagccggccttgcacatgcgttttagagctagaaatagcaagttaaaataaggctagtccgttatcaacttgaaaaagtggcaccgagtcggtgctcgacgacgagcgcggcgatatcatcatccatggtaccgttcgtatagcatacattatacgaagttattgagctgcgagaa

SEQ ID NO: 304 - DNA, 159 bp, synthetic construct
  source 1..159: mol_type = other DNA
gaagccggccttgcacatgcgttttagagctagaaatagcaagttaaaataaggctagtccgttatcaacttgaaaaagtggcaccgagtcggtgcatatcatcatccatggtaccgttcgtatagcatacattatacgaagttattgagctgcgagaa

SEQ ID NO: 305 - DNA, 96 bp, synthetic construct
  source 1..96: mol_type = other DNA
ccccacgatggaggggaagagttttagagctagaaatagcaagttaaaataaggctagtccgttatcaacttgaaaaagtggcaccgagtcggtgc

SEQ ID NO: 306 - DNA, 96 bp, synthetic construct
  source 1..96: mol_type = other DNA
ccttctcctggagccgcgacgttttagagctagaaatagcaagttaaaataaggctagtccgttatcaacttgaaaaagtggcaccgagtcggtgc

SEQ ID NO: 307 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcattatatgttcttacagtatggcggcccggattgtaaaaacatataatgtgagctgcgagaa

SEQ ID NO: 308 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcgttatagggtattacagtatggcggtcggtactgcaataccctataacgtgagctgcgagaa

SEQ ID NO: 309 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgtatcattttcatatagttagcacctgcacactatatgaaaatgatacatgagctgcgagaa

SEQ ID NO: 310 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgtctactatctgtatatgcgacacatgtggcataaagacatagtagacatgagctgcgagaa

SEQ ID NO: 311 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcatcgaccctgacgcatgcggaggcggcgctccatgcgtctgacctcatttgagctgcgagaa

SEQ ID NO: 312 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggttagtacccaaatgacaaaaggtcatccttttatcatttgggtactaactgagctgcgagaa

SEQ ID NO: 313 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcttattaaaacccgttccgcttctgtcaaagcggcatcggttttataaactgagctgcgagaa

SEQ ID NO: 314 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggcgtgatggtcgtgaacctcaacatgacgacgaacacgacctcgcggcctgagctgcgagaa

SEQ ID NO: 315 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtctacatcttgaatatatcaagttataactttgaattatatcagtttatatgagctgcgagaa

SEQ ID NO: 316 - DNA, 188 bp, synthetic construct
  source 1..188: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggaattatatctaaaagcactaagctccgccatactgcttttagatataatatgagctgcgagaa

SEQ ID NO: 317 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggatatggggaagtgaatcagtacaaccgccacagtacctgagctgcgagaa

SEQ ID NO: 318 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggtactgtggcggttgtactgattcacttccccatatctgagctgcgagaa

SEQ ID NO: 319 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgggtggtacaggtgccacattagttgtaccatttatgtgagctgcgagaa

SEQ ID NO: 320 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcataaatggtacaactaatgtggcacctgtaccacccatgagctgcgagaa

SEQ ID NO: 321 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggttgtttttccagatccagttggtcctgtaaatataagtgagctgcgagaa

SEQ ID NO: 322 - DNA, 176 bp, synthetic construct
  source 1..176: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggcttatatttacaggaccaactggatctggaaaaacaactgagctgcgagaa

SEQ ID NO: 323 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggtactgtggcggttgtactgattcacttccccatattgagctgcgagaa

SEQ ID NO: 324 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtactgtggcggttgtactgattcacttccccatatgagctgcgagaa

SEQ ID NO: 325 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggactgtggcggttgtactgattcacttccccattgagctgcgagaa

SEQ ID NO: 326 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatatggggaagtgaatcagtacaaccgccacagtactgagctgcgagaa

SEQ ID NO: 327 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtatggggaagtgaatcagtacaaccgccacagtatgagctgcgagaa

SEQ ID NO: 328 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatggggaagtgaatcagtacaaccgccacagttgagctgcgagaa

SEQ ID NO: 329 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggataaatggtacaactaatgtggcacctgtaccaccctgagctgcgagaa

SEQ ID NO: 330 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtaaatggtacaactaatgtggcacctgtaccacctgagctgcgagaa

SEQ ID NO: 331 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggaaatggtacaactaatgtggcacctgtaccactgagctgcgagaa

SEQ ID NO: 332 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggggtggtacaggtgccacattagttgtaccatttattgagctgcgagaa

SEQ ID NO: 333 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggggtggtacaggtgccacattagttgtaccatttatgagctgcgagaa

SEQ ID NO: 334 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggtggtacaggtgccacattagttgtaccattttgagctgcgagaa

SEQ ID NO: 335 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggttatatttacaggaccaactggatctggaaaaacaatgagctgcgagaa

SEQ ID NO: 336 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtatatttacaggaccaactggatctggaaaaacatgagctgcgagaa

SEQ ID NO: 337 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggatatttacaggaccaactggatctggaaaaactgagctgcgagaa

SEQ ID NO: 338 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggttgtttttccagatccagttggtcctgtaaatataatgagctgcgagaa

SEQ ID NO: 339 - DNA, 172 bp, synthetic construct
  source 1..172: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatggtgtttttccagatccagttggtcctgtaaatatatgagctgcgagaa

SEQ ID NO: 340 - DNA, 170 bp, synthetic construct
  source 1..170: mol_type = other DNA
gctattctcgcagctcaccagttttagagctagaaatagcaagttaaaataaggctagtccgttatcaacttgaaaaagtggcaccgagtcggtgcgacgagcgcggcgatatcatcatccatgggtttttccagatccagttggtcctgtaaatattgagctgcgagaa

SEQ ID NO: 341 - DNA, 151 bp, synthetic construct
  source 1..151: mol_type = other DNA
gctgtctccgccgcccgccagttttagagctagaaatagcaagttaaaataaggctagtccgttatcaacttgaaaaagtggcaccgagtcggtgcagtcgccatgatatggggaagtgaatcagtacaaccgccacagtaccgggcggcg

SEQ ID NO: 342 - DNA, 179 bp, synthetic construct
  source 1..179: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccatatggggaagtgaatcagtacaaccgccacagtactcctccaggcaatacgcg

SEQ ID NO: 343 - DNA, 170 bp, synthetic construct
  source 1..170: mol_type = other DNA
gagaagcggcgtccggggctagttttagagctagaaatagcaagttaaaataaggctagtccgttatcaacttgaaaaagtggcaccgagtcggtgctctttgtccagagtcacagccataatatggggaagtgaatcagtacaaccgccacagtacccccggacgccgc

SEQ ID NO: 344 - DNA, 169 bp, synthetic construct
  source 1..169: mol_type = other DNA
gggcacggggccatgtacaagttttagagctagaaatagcaagttaaaataaggctagtccgttatcaacttgaaaaagtggcaccgagtcggtgcggcgtcggcagcccgatcccgttgatatggggaagtgaatcagtacaaccgccacagtactacatggccccgt

SEQ ID NO: 345 - DNA, 175 bp, synthetic construct
  source 1..175: mol_type = other DNA
gtgtcaggtggggcggggctagttttagagctagaaatagcaagttaaaataaggctagtccgttatcaacttgaaaaagtggcaccgagtcggtgcgctggctcctcccctggcaccataatatggggaagtgaatcagtacaaccgccacagtacccccgccccacctgacac

SEQ ID NO: 346 - DNA, 174 bp, synthetic construct
  source 1..174: mol_type = other DNA
gagtgggtcagacgagcaggagttttagagctagaaatagcaagttaaaataaggctagtccgttatcaacttgaaaaagtggcaccgagtcggtgccgactcctcccccatgcagccctccatcatatggggaagtgaatcagtacaaccgccacagtactgctcgtctgacc

SEQ ID NO: 347 - DNA, 161 bp, synthetic construct
  source 1..161: mol_type = other DNA
gctgtctccgccgcccgccagttttagagctagaaatagcaagttaaaataaggctagtccgttatcaacttgaaaaagtggcaccgagtcggtgccgggggtcgcagtcgccatgatatggggaagtgaatcagtacaaccgccacagtaccgggcggcg

SEQ ID NO: 348 - DNA, 165 bp, synthetic construct
  source 1..165: mol_type = other DNA
gggcacggggccatgtacaagttttagagctagaaatagcaagttaaaataaggctagtccgttatcaacttgaaaaagtggcaccgagtcggtgccgggggtcgcagtcgccatgatatggggaagtgaatcagtacaaccgccacagtaccgggcggcggaga

SEQ ID NO: 349 - DNA, 174 bp, synthetic construct
  source 1..174: mol_type = other DNA
gggcacggggccatgtacaagttttagagctagaaatagcaagttaaaataaggctagtccgttatcaacttgaaaaagtggcaccgagtcggtgcgcggcggcacgggggtcgcagtcgccatgatatggggaagtgaatcagtacaaccgccacagtaccgggcggcggaga

SEQ ID NO: 350 - DNA, 174 bp, synthetic construct
  source 1..174: mol_type = other DNA
gcgtattgcctggaggatgggttttagagctagaaatagcaagttaaaataaggctagtccgttatcaacttgaaaaagtggcaccgagtcggtgcgaaccacgcggcgaatgccggcgtccgccatatggggaagtgaatcagtacaaccgccacagtactcctccaggcaat

SEQ ID NO: 351 - DNA, 165 bp, synthetic construct
  source 1..165: mol_type = other DNA
gggcacggggccatgtacaagttttagagctagaaatagcaagttaaaataaggctagtccgttatcaacttgaaaaagtggcaccgagtcggtgcggcgaatgccggcgtccgccatatggggaagtgaatcagtacaaccgccacagtactcctccaggcaat

SEQ ID NO: 352 - DNA, 170 bp, synthetic construct
  source 1..170: mol_type = other DNA
gggcacggggccatgtacaagttttagagctagaaatagcaagttaaaataaggctagtccgttatcaacttgaaaaagtggcaccgagtcggtgcggcgaatgccggcgtccgccatatggggaagtgaatcagtacaaccgccacagtactcctccaggcaatacgcg

SEQ ID NO: 353 - DNA, 165 bp, synthetic construct
  source 1..165: mol_type = other DNA
gggcacggggccatgtacaagttttagagctagaaatagcaagttaaaataaggctagtccgttatcaacttgaaaaagtggcaccgagtcggtgcggcgtcggcagcccgatcccgttgatatggggaagtgaatcagtacaaccgccacagtactacatggcc

SEQ ID NO: 354 - DNA, 151 bp, synthetic construct
  source 1..151: mol_type = other DNA
gggcacggggccatgtacaagttttagagctagaaatagcaagttaaaataaggctagtccgttatcaacttgaaaaagtggcaccgagtcggtgcgatcccgttgatatggggaagtgaatcagtacaaccgccacagtactacatggcc

SEQ ID NO: 355 - DNA, 155 bp, synthetic construct
  source 1..155: mol_type = other DNA
gggcacggggccatgtacaagttttagagctagaaatagcaagttaaaataaggctagtccgttatcaacttgaaaaagtggcaccgagtcggtgcgatcccgttgatatggggaagtgaatcagtacaaccgccacagtactacatggccccgt

SEQ ID NO: 356 - DNA, 169 bp, synthetic construct
  source 1..169: mol_type = other DNA
gctattctcgcagctcaccagttttagagctagaaatagcaagttaaaataaggctagtccgttatcaacttgaaaaagtggcaccgagtcggtgcgcgcggcgatatcatcatccatggatgatcctgacgacggagaccgccgtcgtcgacaagcctgagctgcgag

SEQ ID NO: 357 - DNA, 174 bp, synthetic construct
  source 1..174: mol_type = other DNA
gctattctcgcagctcaccagttttagagctagaaatagcaagttaaaataaggctagtccgttatcaacttgaaaaagtggcaccgagtcggtgccgatatcatcatccatggcggatgatcctgacgacggagaccgccgtcgtcgacaagccggctgagctgcgagaatag

SEQ ID NO: 358 - DNA, 168 bp, synthetic construct
  source 1..168: mol_type = other DNA
gctgtctccgccgcccgccagttttagagctagaaatagcaagttaaaataaggctagtccgttatcaacttgaaaaagtggcaccgagtcggtgcgcggcacgggggtcgcagtcgccatgatgatcctgacgacggagaccgccgtcgtcgacaagcccgggcggc

SEQ ID NO: 359 - DNA, 171 bp, synthetic construct
  source 1..171: mol_type = other DNA
gcgtattgcctggaggatgggttttagagctagaaatagcaagttaaaataaggctagtccgttatcaacttgaaaaagtggcaccgagtcggtgcaatgccggcgtccgcccggatgatcctgacgacggagaccgccgtcgtcgacaagccggctcctccaggcaatac

SEQ ID NO: 360 - DNA, 158 bp, synthetic construct
  source 1..158: mol_type = other DNA
gcgtattgcctggaggatgggttttagagctagaaatagcaagttaaaataaggctagtccgttatcaacttgaaaaagtggcaccgagtcggtgcggcgtccgccatgatcctgacgacggagaccgccgtcgtcgacaagcctcctccaggcaata

SEQ ID NO: 361 - DNA, 179 bp, synthetic construct
  source 1..179: mol_type = other DNA
gggaaatgcatcttgcacaagttttagagctagaaatagcaagttaaaataaggctagtccgttatcaacttgaaaaagtggcaccgagtcggtgcagcccctccatgctctctagctgttgccattgggcttgtcgacgacggcggtctccgtcgtcaggatcattgcaagatgcatt

SEQ ID NO: 362 - DNA, 153 bp, synthetic construct
  source 1..153: mol_type = other DNA
gtgtcaggtggggcggggctagttttagagctagaaatagcaagttaaaataaggctagtccgttatcaacttgaaaaagtggcaccgagtcggtgctggcaccataatgatcctgacgacggagaccgccgtcgtcgacaagccccccgccc

SEQ ID NO: 363 - DNA, 96 bp, synthetic construct
  source 1..96: mol_type = other DNA
gtggggacagccccgtctctgttttagagctagaaatagcaagttaaaataaggctagtccgttatcaacttgaaaaagtggcaccgagtcggtgc

SEQ ID NO: 364 - DNA, 97 bp, synthetic construct
  source 1..97: mol_type = other DNA
gctcttgggaaaaaaaccctagttttagagctagaaatagcaagttaaaataaggctagtccgttatcaacttgaaaaagtggcaccgagtcggtgc

SEQ ID NO: 365 - DNA, 97 bp, synthetic construct
  source 1..97: mol_type = other DNA
gtcttgggaaaaaaaccctaagttttagagctagaaatagcaagttaaaataaggctagtccgttatcaacttgaaaaagtggcaccgagtcggtgc

SEQ ID NO: 366 - DNA, 96 bp, synthetic construct
  source 1..96: mol_type = other DNA
gaaaaaaaccctaagggctggttttagagctagaaatagcaagttaaaataaggctagtccgttatcaacttgaaaaagtggcaccgagtcggtgc

SEQ ID NO: 367 - DNA, 97 bp, synthetic construct
  source 1..97: mol_type = other DNA
gctgaggatccttgtgagtgtgttttagagctagaaatagcaagttaaaataaggctagtccgttatcaacttgaaaaagtggcaccgagtcggtgc

SEQ ID NO: 368 - DNA, 97 bp, synthetic construct
  source 1..97: mol_type = other DNA
gtgaggatccttgtgagtgttgttttagagctagaaatagcaagttaaaataaggctagtccgttatcaacttgaaaaagtggcaccgagtcggtgc

SEQ ID NO: 369 - DNA, 96 bp, synthetic construct
  source 1..96: mol_type = other DNA
ggatccttgtgagtgttggggttttagagctagaaatagcaagttaaaataaggctagtccgttatcaacttgaaaaagtggcaccgagtcggtgc

SEQ ID NO: 370 - DNA, 96 bp, synthetic construct
  source 1..96: mol_type = other DNA
gatccttgtgagtgttgggtgttttagagctagaaatagcaagttaaaataaggctagtccgttatcaacttgaaaaagtggcaccgagtcggtgc

SEQ ID NO: 371 - DNA, 96 bp, synthetic construct
  source 1..96: mol_type = other DNA
gttgggtgggaacagctcccgttttagagctagaaatagcaagttaaaataaggctagtccgttatcaacttgaaaaagtggcaccgagtcggtgc

SEQ ID NO: 372 - DNA, 96 bp, synthetic construct
  source 1..96: mol_type = other DNA
gggtgggaacagctcccagggttttagagctagaaatagcaagttaaaataaggctagtccgttatcaacttgaaaaagtggcaccgagtcggtgc

SEQ ID NO: 373 - DNA, 97 bp, synthetic construct
  source 1..97: mol_type = other DNA
gcttctgtgcagcagtttcccgttttagagctagaaatagcaagttaaaataaggctagtccgttatcaacttgaaaaagtggcaccgagtcggtgc

SEQ ID NO: 374 - DNA, 97 bp, synthetic construct
  source 1..97: mol_type = other DNA
gtttccctggccactaaataggttttagagctagaaatagcaagttaaaataaggctagtccgttatcaacttgaaaaagtggcaccgagtcggtgc

SEQ ID NO: 375 - DNA, 97 bp, synthetic construct
  source 1..97: mol_type = other DNA
gttccctggccactaaatagtgttttagagctagaaatagcaagttaaaataaggctagtccgttatcaacttgaaaaagtggcaccgagtcggtgc

SEQ ID NO: 376 - DNA, 96 bp, synthetic construct
  source 1..96: mol_type = other DNA
gattagatagaagccctccagttttagagctagaaatagcaagttaaaataaggctagtccgttatcaacttgaaaaagtggcaccgagtcggtgc

SEQ ID NO: 377 - DNA, 97 bp, synthetic construct
  source 1..97: mol_type = other DNA
gattagatagaagccctccaagttttagagctagaaatagcaagttaaaataaggctagtccgttatcaacttgaaaaagtggcaccgagtcggtgc

SEQ ID NO: 378 - protein, 527 aa, unidentified
MEKNRAVLYLRLSKEDVDKVNKGDDSSSIKSQRLLLTDFALERGFKIVGVYSDDDESGLYDDRPDFERMMTDAKLDEFDIIIAKTQSRFSRNMEHIEKYLHHDLPNLGIRFIGAVDGVDTESDENKKSRQINGLVNEWYCEDLSKNIRSAFKAKMKDGQFLGSSCPYGYKKDPQNHNHLVVDDYAAKVVQKIFNLYLEGYGKAKIGSILSSEGILIPTLYKKDILKQNYHNSKALDTTQNWSYQTIHTILNNEVYLGHLIQNKVNTMSYKDKNKRILPKEKWIIVRNTHEPIITEEMFQDVQKLQKNRTRSVENIEPNGLFSGLIFCADCKHAMSRKYARRGEKGFVGYVCKTYKTQGKNFCESHSIDYDELEEAVLFSIKNEARSILQQEEIDELRKVQAYDETKSYYEMQLENIKSRMEKIEKYKKKTYDNYMDDLISRDDYKKYVTEYDKEIGGLKQQQELINSKTDLEKEISTQYDEWVEAFINYVDIDKLTREIVIELIEKIEVNKDGSINIYYKFKNPYIS

SEQ ID NO: 379 - protein, 510 aa, unidentified
MNTVIYARYSAGPRQTDQSIDGQLRVCTEFCKQRGLTVVDTYCDRHISGRTDERPEFQRLIADAKAHKFEAVVVYKTDRFARNKYDSAIYKRELRRNGIQIFYAAEAIPEGPEGIILESLMEGLAEYYSAELAQKIKRGLNESALKCQSLGSGRPLGYTVDEQKHFQIDPESSQAVKTIFEMYIKGESNAAICDYLNARGLRTSQGNLFNKNSINRIIKNRKYIGEYRYNDIVVEGGMPAIISKETFCMAQAEMERRRTHRAPVSPKAEYLLAGKLFCGHCKGPMQGVSGTGKSGNKWYYYYCANTRGKERTCDKKQVSRDRLEKAVVDFTVRYILQENVLEELSKKVYAAQERQNNTASEIAFYEKKLAENKKAIANILRAIESGAMTQALPARLQELENEQTVIQGELSYLKGARLAFTEDQILFALLQHLDPRPGESERDYHRRIITDFVSEVYLYDDRMLIYFNISSADGKLKHADLSAIESGVFDAGLISSSSRASSFSTRCALI

SEQ ID NO: 380 - protein, 482 aa, unidentified
MNEKNLEIGAAYIRVSTDDQTELSPDAQLRVILEAAKKDGIIIPQEFVFMEDRGRSGRRADNRPEFQRMISTARQNPSPFRYLYLWKFSRFARNQEESAFYKGILRKKCGVTIKSVSEPIMEGMFGRLVEMIIEWSDEFYSVNLSGEVLRGMTQKALEHGYQLTPCLGYDAVGHGRPYVINEEQYQIVEFIHRSFFDGKDMTWIAREANRRGYHTRRGNPFDTRAVRIILTNSFYVGLVKWNDVTFQGTHECRESVTSVFSANQERLNRIHRPRGRRQASSCKHWLSGLLKCSICGASLGYNQTKDLTKRGHAFQCWKYTKGIHPGSCSVSSLKAEAAVLESLQMILETGEVEYTYEQREKHLDDNKLTLIQKSLERLDTKELRIREAYESGIDTLDEFKTNKARLQRERDQLMEELEELHSQEEPEDVPGKEILIERIQNVYDLLQSPDVDNDDKGNAVRSIIKKIVYIKESKTFCFYYYV

SEQ ID NO: 381 - protein, 406 aa, unidentified
MERTIKVIQPGTVKIPTKKRVAAYARVSSGKDAMLHSLSAQVSYYSNMIQQKNEWSYVGIYADEAITGTKDRRVEFNRLIQDCTDGKIDMIITKSISRFARNTLTMLEVVRKLKNINVDVYFEKENIHSISGDGELMLTILASFAQEESRSVSENCKWRIRKGFEQGELINLRFLYGYRINKGKIEIYEKEAEIVRMIFDDYLNGEGCTRIGNKLRKMKVNKLRGGMWNSERVVDIIKNEKYTGNALLQKKYVKDHLSKKLVRNKGILTQYYAEGTHPAIIDIKTFEIAQKIMEANRTKFQGKCGSNRYLFTSKIECGICGKNYRHKDREGKSTWVCANHLKYGNSRCIAKPLNEEKLKKLINEALELKYFDEEIFIRNIKRIKVTGNQTIEFILKDGKVIEEGMI

SEQ ID NO: 382 - protein, 401 aa, unidentified
MKKIKIDRAIQERPATRKQTRNEKIRQSLTEHVDVQVIPAITDREGYEKPKLRVCAYCRVSTDMDTQALSYELQVQNYTDYIRGNDEWRFAGIYADRGISGTSLKHRDEFNRMIEDCKAGKIDLIITKAVTRFARNVLDCISTIRMLKQLEHPVAVYFETERINTLDTTSETYLGLISLFAQGESESKSESLKWSYIRRWKRGTGIYPAWSLLGYEMGEDGKWQIVEAEAELVRIIYDMYLNGYSSPQIAEILTRSGVPTATNQTVWSSGGVLGILRNEKYCGNVLCQKTMTVDVFSHKAIKNTGQKTQYFIEGHHDPIILRSDWDRVQQMIDEKYYRKRRGRRTKPRIVLKGCLAGFTQIDLDWDEDDIARIFYSTTPAAEVATPAMADHIEIIKVKGEN

SEQ ID NO: 383 - protein, 476 aa, unidentified
MKTAAAYIRVSTDDQVEYSPDSQIKLIRDYAKRNDYILPDEFIFRDDGISGKSAKHRPEFTKMIALAKSPEHPFDAILVWKFSRFARNQEESIVFKNILRKIGVEVRSVSEPISEDPFGSLVERIIEWTDEYYIINLSGEVKRGMLEKISRGQPVVPPPVGYKMENGQYIPDENAHFIKEIFEAYAAGEGARHIAQRLAAQGCLTKRGNPIDNRFVDYVLHNPVYIGKLRWSVNSHAASSRHYDSADIIVFDGTHEPLISSELWESVQKRLHEVKTLYPKYQRREQPVSFMLKGLVRCSSCGSTLCYCRTSEPSLQCHSYARGSCRQSHSINIATANEAVIKGLQLAVDKLDFAIAPAKPHYSADAPGTNKLLAAEYKKMERIKAAYANGTDTLEEYAANKKKISAEIARLEAELQQESNVKPINKKAFAKRVSEIIKYISDPHNSEAAKNQALRTVISYIIFDRAATTFNIIFHF

SEQ ID NO: 384 - protein, 550 aa, unidentified
MKIAIYARKSKYSPTGESVENQIQLCKEYLQAKYKSETLEIDEYKDEGYSGGNTNRPDFKKLIAQIEDYDMLICYRLDRISRNVADFSSTLTLLQNNKCDFVSIKEQFDTTSPMGRAMIYISSVFAQLERETIAERIRDNMMELAKMGRWLGGTIPMGFDSEPITFIDENMKERSMTKLIPNVEELKVIELIYEKYLQLGSMGKVVTYLLQNNIKTKKGKDFTLGSIKVILTNPIYVKANQEVVNHLKTQGITICGDVDGKKALLTYNKTTGISNDVGTKTIVKDKSEWIAAVANHKGIIPADKWLQAQNIKDKNKDSFPALGRSNTTIASRVLRCDKCESTMGVTHGHINPVTGKKHYYYNCTLKKRSKGVRCDNKPAKAAEVDEAILITLENMFKAKSSIIDNLKAKNKARRIEMISSNRVDVINKIIEDKTKQIDNLVNKLSLDDDLTDILFKKIKGLKAEIKELEDELLTLTSDNIKLNEDEVVLDFTEKLLEKCSIIRTLDILEQQQIVDALIPLVTWNGDTEVLNIYPLGSPELELKEAESKKK

SEQ ID NO: 385 - protein, 493 aa, unidentified
MKEKVSERKTGAIYIRVSTDKQEELSPDAQLRLLLDYAKKDSIDVPKEYIFQDNGISGRKANKRPAFQNMIALAKSKEHPIDTIIVWKFSRFARNQEESIVYKSLLKKNNVDVVSVSEPLIDGPFGSLIERIIEWMDEYYSIRLSGEVMRGMTQNAMRGHYQSDAPIGYTSPGDKKPPVINPDTVQIPLMIKDMFLSGSTQLQIARKLNDSGYRTKRGNLWDARGVRYVLENPFYIGKSRWNYTERGRRLKPADEVIYADGNWEALWDEDTFKEIQKRLALNMRKSKSRDISAAKHWLSGLLICSSCGGTLAFGGAHNMRGFQCWKYSKGFCSESHYISTGPIEKMVLEYLEAVMHSPALSYTVISSSSVDASSKLSDLERQLQKIDAKEKRIKAAYLNEIDTLEEYKANKTALEEERRTVEKEIEELTLSDVKYSKEDLDKKMKQNISDLLRVLRDESADYIQKGNMMRNVVDHIVFNRKNTSLDVFLKLVV

SEQ ID NO: 386 - protein, 404 aa, unidentified
MKITKKQPLRPRGRSEDKRQSTKNVIRDAYINGPQKEVQIIPAKRDMEAETEKKKLRVCAYCRVSTDEDTQASSYELQVQNYTRMIRENPEWEFAGIFADEGISGTSVLHREHFLEMIEKCKAGEIDLIITKQVSRFARNVLDSLNYIFMLRKLDPPVGVYFETEKLNTLDKSSDMVITVLSLVAQSESEQKSNSLKWSFKRRRAQGLGIYPSWALLGYRLDDEKNWEIVEDEADIVRTIYSLYLDGYSSTQIAELLTKSGIPTVKGLSVWSSGSVLGILKNEKFCGDALCQKTVTIDFFTHKSVKNNGIEPQYFVEGHHIPIIEKNDWLLAQQIRKERRYRKRRSTHRKPRIVVKGALSGFMIVDTSWDEEYVDSLLISATQKPEPAPVIAEEDENFIVIEKE

SEQ ID NO: 387 - protein, 498 aa, unidentified
MADIQPVKNGALYIRVSTHLQEELSPDAQKRLLMEYAEAHNIIVLKEHIYIDSGISGRSARQRPQFNNMIAEAKSKEHPFDVILVWKYSRFARNQEESIVYKSMLKRENVDVISVSEPISDDPFGSLIERIIEWMDEYYSIRLSGEVSRGMAENAMRGNYQARPPLGYRIPGYRQTPVIVPEEAELIQLIFDLYTEKKMGIFEIVRYLNEHGYQTGHKKPFQRRSVTYILKNPTYIGKTIWNQHDQDHKLRDKSEWIIADGKHEPIISKEQFDKAQKRIESTYKPAYRKPTSVCHHWLSSLLKCSSCGRTLVVKRTASKKKDRMYVNFQCYGYQKGICNTNQSISAIKLEPVIMHALEDAMTSGKIHFDVLNPTTLDSSQKQQFLTRLNEIEKKEERIKRAYRDGIDTLEEYKENKSIIQTEKEMLLKKIEHIEEPALSPEEAKPIMMDRIKNVYEIITNPDIGMEEKNKAARSIIEKIVFDRATGSVNIFFYLAHCP

SEQ ID NO: 388 - protein, 500 aa, unidentified [sequence omitted]

SEQ ID NO: 389 - protein, 485 aa, unidentified
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 390 - protein, 594 aa, unidentified
MSPFIAPDVPEHLLDTVRVFLYARQSKGRSDGSDVSTEAQLAAGRALVASRNAQGGARWVVAGEFVDVGRSGWDPNVTRADFERMMGEVRAGEGDVVVVNELSRLTRKGAHDALEIDNELKKHGVRFMSVLEPFLDTSTPIGVAIFALIAALAKQDSDLKAERLKGAKDEIAALGGVHSSSAPFGMRAVRKKVDNLVISVLEPDEDNPDHVELVERMAKMSFEGVSDNAIATTFEKEKIPSPGMAERRATEKRLASIKARRLNGAEKPIMWRAQTVRWILNHPAIGGFAFERVKHGKAHINVIRRDPGGKPLTPHTGILSGSKWLELQEKRSGKNLSDRKPGAEVEPTLLSGWRFLGCRICGGSMGQSQGGRKRNGDLAEGNYMCANPKGHGGLSVKRSELDEFVASKVWARLRTADMEDEHDQAWIAAAAERFALQHDLAGVADERREQQAHLDNVRRSIKDLQADRKAGLYVGREELETWRSTVLQYRSYEAECTTRLAELDEKMNGSTRVPSEWFSGEDPTAEGGIWASWDVYERREFLSFFLDSVMVDRGRHPETKKYIPLKDRVTLKWAELLKEEDEASEATERELAAL

SEQ ID NO: 391 - protein, 528 aa, Bacillus cereus
MYPYDVPDYAGSYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKIS

SEQ ID NO: 392 - protein, 486 aa, unidentified
MYPYDVPDYAGSAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQS

SEQ ID NO: 393 - protein, 472 aa, Staphylococcus lugdunensis
MYPYDVPDYAGSKVAIYTRVSSAEQANEGYSIHEQKKKLISYCEIHDWNEYKVFTDAGISGGSMKRPALQKLMKHLSSFDLVLVYKLDRLTRNVRDLLDMLEEFEQYNVSFKSATEVFDTTSAIGKLFITMVGAMAEWERETIRERSLFGSRAAVREGNYIREAPFCYDNIEGKLHPNEYAKVIDLIVSMFKKGISANEIARRLNSSKVHVPNKKSWNRNSLIRLMRSPVLRGHTKYGDMLIENTHEPVLSEHDYNAINNAISSKTHKSKVKHHAIFRGALVCPQCNRRLHLYAGTVKDRKGYKYDVRRYKCETCSKNKDVKNVSFNESEVENKFVNLLKSYELNKFHIRKVEPVKKIEYDIDKINKQKINYTRSWSLGYIEDDEYFELMEEINATKKMIEEQTTENKQSVSKEQIQSINNFILKGWEELTIKDKEELILSTVDKIEFNFIPKDKKHKTNTLDINNIHFKFS

SEQ ID NO: 394 - DNA, 52 bp, unidentified
  source 1..52: mol_type = unassigned DNA
gtggtttgtctggtcaaccaccgcggtctcagtggtgtacggtacaaaccca

SEQ ID NO: 395 - DNA, 52 bp, synthetic construct
  source 1..52: mol_type = other DNA
gtggtttgtctggtcaaccaccgcgcgctcagtggtgtacggtacaaaccca

SEQ ID NO: 396 - DNA, 52 bp, synthetic construct
  source 1..52: mol_type = other DNA
gtggtttgtctggtcaaccaccgcggcctcagtggtgtacggtacaaaccca

SEQ ID NO: 397 - DNA, 52 bp, synthetic construct
  source 1..52: mol_type = other DNA
gtggtttgtctggtcaaccaccgcgatctcagtggtgtacggtacaaaccca

SEQ ID NO: 398 - DNA, 52 bp, synthetic construct
  source 1..52: mol_type = other DNA
gtggtttgtctggtcaaccaccgcgtactcagtggtgtacggtacaaaccca

SEQ ID NO: 399 - DNA, 52 bp, synthetic construct
  source 1..52: mol_type = other DNA
gtggtttgtctggtcaaccaccgcgggctcagtggtgtacggtacaaaccca

SEQ ID NO: 400 - DNA, 52 bp, synthetic construct
  source 1..52: mol_type = other DNA
gtggtttgtctggtcaaccaccgcgttctcagtggtgtacggtacaaaccca

SEQ ID NO: 401 - DNA, 52 bp, synthetic construct
  source 1..52: mol_type = other DNA
gtggtttgtctggtcaaccaccgcggactcagtggtgtacggtacaaaccca

SEQ ID NO: 402 - DNA, 52 bp, synthetic construct
  source 1..52: mol_type = other DNA
gtggtttgtctggtcaaccaccgcgagctcagtggtgtacggtacaaaccca

SEQ ID NO: 403 - DNA, 52 bp, synthetic construct
  source 1..52: mol_type = other DNA
gtggtttgtctggtcaaccaccgcgccctcagtggtgtacggtacaaaccca

SEQ ID NO: 404 - DNA, 52 bp, synthetic construct
  source 1..52: mol_type = other DNA
gtggtttgtctggtcaaccaccgcgtcctcagtggtgtacggtacaaaccca

SEQ ID NO: 405 - DNA, 52 bp, synthetic construct
  source 1..52: mol_type = other DNA
gtggtttgtctggtcaaccaccgcgctctcagtggtgtacggtacaaaccca

SEQ ID NO: 406 - DNA, 52 bp, synthetic construct
  source 1..52: mol_type = other DNA
gtggtttgtctggtcaaccaccgcgaactcagtggtgtacggtacaaaccca

SEQ ID NO: 407 - DNA, 52 bp, synthetic construct
  source 1..52: mol_type = other DNA
gtggtttgtctggtcaaccaccgcgcactcagtggtgtacggtacaaaccca

SEQ ID NO: 408 - DNA, 52 bp, synthetic construct
  source 1..52: mol_type = other DNA
gtggtttgtctggtcaaccaccgcgacctcagtggtgtacggtacaaaccca

SEQ ID NO: 409 - DNA, 52 bp, synthetic construct
  source 1..52: mol_type = other DNA
gtggtttgtctggtcaaccaccgcgtgctcagtggtgtacggtacaaaccca

SEQ ID NO: 410 - DNA, 46 bp, synthetic construct
  source 1..46: mol_type = other DNA
ggccggcttgtcgacgacggcggtctccgtcgtcaggatcatccgg

SEQ ID NO: 411 - DNA, 46 bp, synthetic construct
  source 1..46: mol_type = other DNA
ggccggcttgtcgacgacggcgaactccgtcgtcaggatcatccgg

SEQ ID NO: 412 - DNA, 46 bp, synthetic construct
  source 1..46: mol_type = other DNA
ggccggcttgtcgacgacggcggactccgtcgtcaggatcatccgg

SEQ ID NO: 413 - DNA, 46 bp, synthetic construct
  source 1..46: mol_type = other DNA
ggccggcttgtcgacgacggcgcactccgtcgtcaggatcatccgg

SEQ ID NO: 414 - DNA, 46 bp, synthetic construct
  source 1..46: mol_type = other DNA
ggccggcttgtcgacgacggcgtactccgtcgtcaggatcatccgg

SEQ ID NO: 415 - DNA, 46 bp, synthetic construct
  source 1..46: mol_type = other DNA
ggccggcttgtcgacgacggcgagctccgtcgtcaggatcatccgg

SEQ ID NO: 416 - DNA, 46 bp, synthetic construct
  source 1..46: mol_type = other DNA
ggccggcttgtcgacgacggcgggctccgtcgtcaggatcatccgg

SEQ ID NO: 417 - DNA, 46 bp, synthetic construct
  source 1..46: mol_type = other DNA
ggccggcttgtcgacgacggcgcgctccgtcgtcaggatcatccgg

SEQ ID NO: 418 - DNA, 46 bp, synthetic construct
  source 1..46: mol_type = other DNA
ggccggcttgtcgacgacggcgtgctccgtcgtcaggatcatccgg

SEQ ID NO: 419 - DNA, 46 bp, synthetic construct
  source 1..46: mol_type = other DNA
ggccggcttgtcgacgacggcgacctccgtcgtcaggatcatccgg

SEQ ID NO: 420 - DNA, 46 bp, synthetic construct
  source 1..46: mol_type = other DNA
ggccggcttgtcgacgacggcggcctccgtcgtcaggatcatccgg

SEQ ID NO: 421 - DNA, 46 bp, synthetic construct
  source 1..46: mol_type = other DNA
ggccggcttgtcgacgacggcgccctccgtcgtcaggatcatccgg

SEQ ID NO: 422 - DNA, 46 bp, synthetic construct
  source 1..46: mol_type = other DNA
ggccggcttgtcgacgacggcgtcctccgtcgtcaggatcatccgg

SEQ ID NO: 423 - DNA, 46 bp, synthetic construct
  source 1..46: mol_type = other DNA
ggccggcttgtcgacgacggcgatctccgtcgtcaggatcatccgg

SEQ ID NO: 424 - DNA, 46 bp, synthetic construct
  source 1..46: mol_type = other DNA
ggccggcttgtcgacgacggcgctctccgtcgtcaggatcatccgg

SEQ ID NO: 425 - DNA, 46 bp, synthetic construct
  source 1..46: mol_type = other DNA
ggccggcttgtcgacgacggcgttctccgtcgtcaggatcatccgg

SEQ ID NO: 426 - DNA, 38 bp, synthetic construct
  source 1..38: mol_type = other DNA
ggcttgtcgacgacggcggtctccgtcgtcaggatcat

SEQ ID NO: 427 - DNA, 38 bp, synthetic construct
  source 1..38: mol_type = other DNA
ggcttgtcgacgacggcgaactccgtcgtcaggatcat

SEQ ID NO: 428 - DNA, 38 bp, synthetic construct
  source 1..38: mol_type = other DNA
ggcttgtcgacgacggcggactccgtcgtcaggatcat

SEQ ID NO: 429 - DNA, 38 bp, synthetic construct
  source 1..38: mol_type = other DNA
ggcttgtcgacgacggcgcactccgtcgtcaggatcat

SEQ ID NO: 430 - DNA, 38 bp, synthetic construct
  source 1..38: mol_type = other DNA
ggcttgtcgacgacggcgtactccgtcgtcaggatcat

SEQ ID NO: 431 - DNA, 38 bp, synthetic construct
  source 1..38: mol_type = other DNA
ggcttgtcgacgacggcgagctccgtcgtcaggatcat

SEQ ID NO: 432 - DNA, 38 bp, synthetic construct
  source 1..38: mol_type = other DNA
ggcttgtcgacgacggcgggctccgtcgtcaggatcat

SEQ ID NO: 433 - DNA, 38 bp, synthetic construct
  source 1..38: mol_type = other DNA
ggcttgtcgacgacggcgcgctccgtcgtcaggatcat

SEQ ID NO: 434 - DNA, 38 bp, synthetic construct
  source 1..38: mol_type = other DNA
ggcttgtcgacgacggcgtgctccgtcgtcaggatcat

SEQ ID NO: 435 - DNA, 38 bp, synthetic construct
  source 1..38: mol_type = other DNA
ggcttgtcgacgacggcgacctccgtcgtcaggatcat

SEQ ID NO: 436 - DNA, 38 bp, synthetic construct
  source 1..38: mol_type = other DNA
ggcttgtcgacgacggcggcctccgtcgtcaggatcat

SEQ ID NO: 437 - DNA, 38 bp, synthetic construct
  source 1..38: mol_type = other DNA
ggcttgtcgacgacggcgccctccgtcgtcaggatcat

SEQ ID NO: 438 - DNA, 38 bp, synthetic construct
  source 1..38: mol_type = other DNA
ggcttgtcgacgacggcgtcctccgtcgtcaggatcat

SEQ ID NO: 439 - DNA, 38 bp, synthetic construct
  source 1..38: mol_type = other DNA
ggcttgtcgacgacggcgatctccgtcgtcaggatcat

SEQ ID NO: 440 - DNA, 38 bp, synthetic construct
  source 1..38: mol_type = other DNA
ggcttgtcgacgacggcgctctccgtcgtcaggatcat

SEQ ID NO: 441 - DNA, 38 bp, synthetic construct
  source 1..38: mol_type = other DNA
ggcttgtcgacgacggcgttctccgtcgtcaggatcat

SEQ ID NO: 442 - DNA, 34 bp, synthetic construct
  source 1..34: mol_type = other DNA
taccgttcgtataatgtatgctatacgaagttat

SEQ ID NO: 443 - DNA, 34 bp, synthetic construct
  source 1..34: mol_type = other DNA
ataacttcgtataatgtatgctatacgaacggta

SEQ ID NO: 444 - DNA, 27 bp, synthetic construct
  source 1..27: mol_type = other DNA
tttaccttgattgagatgttaattgtg

SEQ ID NO: 445 - DNA, 50 bp, synthetic construct
  source 1..50: mol_type = other DNA
gcgagtttttatttcgtttatttcaattaaggtaactaaaaaactccttt

SEQ ID NO: 446 - DNA, 34 bp, synthetic construct
  source 1..34: mol_type = other DNA
ctggatcatctggatcactttcgtcaaaaacctg

SEQ ID NO: 447 - DNA, 57 bp, synthetic construct
  source 1..57: mol_type = other DNA
ttcgggtgctgggttgttgtctctggacagtgatccatgggaaactactcagcacca

SEQ ID NO: 448 - DNA, 38 bp, synthetic construct
  source 1..38: mol_type = other DNA
gatatggggaagtgaatcagtacaaccgccacagtacc

SEQ ID NO: 449 - DNA, 38 bp, synthetic construct
  source 1..38: mol_type = other DNA
tgggtggtacaggtgccacattagttgtaccatttatg

SEQ ID NO: 450 - DNA, 38 bp, synthetic construct
  source 1..38: mol_type = other DNA
gttgtttttccagatccagttggtcctgtaaatataag

SEQ ID NO: 451 - DNA, 30 bp, synthetic construct
  source 1..30: mol_type = other DNA
tggggaagtgaatcagtacaaccgccacag

SEQ ID NO: 452 - DNA, 28 bp, synthetic construct
  source 1..28: mol_type = other DNA
ggggaagtgaatcagtacaaccgccaca

SEQ ID NO: 453 - DNA, 26 bp, synthetic construct
  source 1..26: mol_type = other DNA
gggaagtgaatcagtacaaccgccac

SEQ ID NO: 454 - DNA, 30 bp, synthetic construct
  source 1..30: mol_type = other DNA
ctgtggcggttgtactgattcacttcccca

SEQ ID NO: 455 - DNA, 28 bp, synthetic construct
  source 1..28: mol_type = other DNA
tgtggcggttgtactgattcacttcccc

SEQ ID NO: 456 - DNA, 26 bp, synthetic construct
  source 1..26: mol_type = other DNA
gtggcggttgtactgattcacttccc

SEQ ID NO: 457 - DNA, 30 bp, synthetic construct
  source 1..30: mol_type = other DNA
tttttccagatccagttggtcctgtaaata

SEQ ID NO: 458 - DNA, 28 bp, synthetic construct
  source 1..28: mol_type = other DNA
ttttccagatccagttggtcctgtaaat

SEQ ID NO: 459 - DNA, 26 bp, synthetic construct
  source 1..26: mol_type = other DNA
tttccagatccagttggtcctgtaaa

SEQ ID NO: 460 - DNA, 30 bp, synthetic construct
  source 1..30: mol_type = other DNA
tatttacaggaccaactggatctggaaaaa

SEQ ID NO: 461 - DNA, 28 bp, synthetic construct
  source 1..28: mol_type = other DNA
atttacaggaccaactggatctggaaaa

SEQ ID NO: 462 - DNA, 26 bp, synthetic construct
  source 1..26: mol_type = other DNA
tttacaggaccaactggatctggaaa

SEQ ID NO: 463 - DNA, 50 bp, synthetic construct
  source 1..50: mol_type = other DNA
cattatatgtttttacaatccgggccgccatactgtaagaacatataatg

SEQ ID NO: 464 - DNA, 50 bp, synthetic construct
  source 1..50: mol_type = other DNA
cgttatagggtattgcagtaccgaccgccatactgtaataccctataacg

SEQ ID NO: 465 - DNA, 50 bp, synthetic construct
  source 1..50: mol_type = other DNA
tgtatcattttcatatagtgtgcaggtgctaactatatgaaaatgataca

SEQ ID NO: 466 - DNA, 50 bp, synthetic construct
  source 1..50: mol_type = other DNA
tgtctactatgtctttatgccacatgtgtcgcatatacagatagtagaca

SEQ ID NO: 467 - DNA, 50 bp, synthetic construct
  source 1..50: mol_type = other DNA
aatgaggtcagacgcatggagcgccgcctccgcatgcgtcagggtcgatg

SEQ ID NO: 468 - DNA, 50 bp, synthetic construct
  source 1..50: mol_type = other DNA
gttagtacccaaatgataaaaggatgaccttttgtcatttgggtactaac

SEQ ID NO: 469 - DNA, 50 bp, synthetic construct
  source 1..50: mol_type = other DNA
gtttataaaaccgatgccgctttgacagaagcggaacgggttttaataag

SEQ ID NO: 470 - DNA, 50 bp, synthetic construct
  source 1..50: mol_type = other DNA
ggccgcgaggtcgtgttcgtcgtcatgttgaggttcacgaccatcacgcc

SEQ ID NO: 471 - DNA, 50 bp, synthetic construct
  source 1..50: mol_type = other DNA
tataaactgatataattcaaagttataacttgatatattcaagatgtaga

SEQ ID NO: 472 - DNA, 50 bp, synthetic construct
  source 1..50: mol_type = other DNA
tattatatctaaaagcagtatggcggagcttagtgcttttagatataatt

SEQ ID NO: 473 - DNA, 52 bp, unidentified
  source 1..52: mol_type = unassigned DNA
tgggtttgtaccgtacaccactgagaccgcggtggttgaccagacaaaccac

SEQ ID NO: 474 - DNA, 52 bp, synthetic construct
  source 1..52: mol_type = other DNA
tgggtttgtaccgtacaccactgagcgcgcggtggttgaccagacaaaccac

SEQ ID NO: 475 - DNA, 52 bp, synthetic construct
  source 1..52: mol_type = other DNA
tgggtttgtaccgtacaccactgaggccgcggtggttgaccagacaaaccac

SEQ ID NO: 476 - DNA, 52 bp, synthetic construct
  source 1..52: mol_type = other DNA
tgggtttgtaccgtacaccactgagatcgcggtggttgaccagacaaaccac

SEQ ID NO: 477 - DNA, 52 bp, synthetic construct
  source 1..52: mol_type = other DNA
tgggtttgtaccgtacaccactgagtacgcggtggttgaccagacaaaccac

SEQ ID NO: 478 - DNA, 52 bp, synthetic construct
  source 1..52: mol_type = other DNA
tgggtttgtaccgtacaccactgagcccgcggtggttgaccagacaaaccac

SEQ ID NO: 479 - DNA, 52 bp, synthetic construct
  source 1..52: mol_type = other DNA
tgggtttgtaccgtacaccactgagaacgcggtggttgaccagacaaaccac

SEQ ID NO: 480 - DNA, 52 bp, synthetic construct
  source 1..52: mol_type = other DNA
tgggtttgtaccgtacaccactgagtccgcggtggttgaccagacaaaccac

SEQ ID NO: 481 - DNA, 52 bp, synthetic construct
  source 1..52: mol_type = other DNA
tgggtttgtaccgtacaccactgagctcgcggtggttgaccagacaaaccac

SEQ ID NO: 482 - DNA, 52 bp, synthetic construct
  source 1..52: mol_type = other DNA
tgggtttgtaccgtacaccactgagggcgcggtggttgaccagacaaaccac

SEQ ID NO: 483 - DNA, 52 bp, synthetic construct
  source 1..52: mol_type = other DNA
tgggtttgtaccgtacaccactgaggacgcggtggttgaccagacaaaccac

SEQ ID NO: 484 - DNA, 52 bp, synthetic construct
  source 1..52: mol_type = other DNA
tgggtttgtaccgtacaccactgagagcgcggtggttgaccagacaaaccac

SEQ ID NO: 485 - DNA, 52 bp, synthetic construct
  source 1..52: mol_type = other DNA
tgggtttgtaccgtacaccactgagttcgcggtggttgaccagacaaaccac

SEQ ID NO: 486 - DNA, 52 bp, synthetic construct
  source 1..52: mol_type = other DNA
tgggtttgtaccgtacaccactgagtgcgcggtggttgaccagacaaaccac

SEQ ID NO: 487 - DNA, 52 bp, synthetic construct
  source 1..52: mol_type = other DNA
tgggtttgtaccgtacaccactgaggtcgcggtggttgaccagacaaaccac

SEQ ID NO: 488 - DNA, 52 bp, synthetic construct
  source 1..52: mol_type = other DNA
tgggtttgtaccgtacaccactgagcacgcggtggttgaccagacaaaccac

SEQ ID NO: 489 - DNA, 46 bp, synthetic construct
  source 1..46: mol_type = other DNA
ccggatgatcctgacgacggagaccgccgtcgtcgacaagccggcc

SEQ ID NO: 490 - DNA, 46 bp, synthetic construct
  source 1..46: mol_type = other DNA
ccggatgatcctgacgacggagttcgccgtcgtcgacaagccggcc

SEQ ID NO: 491 - DNA, 46 bp, synthetic construct
  source 1..46: mol_type = other DNA
ccggatgatcctgacgacggagtccgccgtcgtcgacaagccggcc

SEQ ID NO: 492 - DNA, 46 bp, synthetic construct
  source 1..46: mol_type = other DNA
ccggatgatcctgacgacggagtgcgccgtcgtcgacaagccggcc

SEQ ID NO: 493 - DNA, 46 bp, synthetic construct
  source 1..46: mol_type = other DNA
ccggatgatcctgacgacggagtacgccgtcgtcgacaagccggcc

SEQ ID NO: 494 - DNA, 46 bp, synthetic construct
  source 1..46: mol_type = other DNA
ccggatgatcctgacgacggagctcgccgtcgtcgacaagccggcc

SEQ ID NO: 495 - DNA, 46 bp, synthetic construct
  source 1..46: mol_type = other DNA
ccggatgatcctgacgacggagcccgccgtcgtcgacaagccggcc

SEQ ID NO: 496 - DNA, 46 bp, synthetic construct
  source 1..46: mol_type = other DNA
ccggatgatcctgacgacggagcgcgccgtcgtcgacaagccggcc

SEQ ID NO: 497 - DNA, 46 bp, synthetic construct
  source 1..46: mol_type = other DNA
ccggatgatcctgacgacggagcacgccgtcgtcgacaagccggcc

SEQ ID NO: 498 - DNA, 46 bp, synthetic construct
  source 1..46: mol_type = other DNA
ccggatgatcctgacgacggaggtcgccgtcgtcgacaagccggcc

SEQ ID NO: 499 - DNA, 46 bp, synthetic construct
  source 1..46: mol_type = other DNA
ccggatgatcctgacgacggaggccgccgtcgtcgacaagccggcc

SEQ ID NO: 500 - DNA, 46 bp, synthetic construct
  source 1..46: mol_type = other DNA
ccggatgatcctgacgacggagggcgccgtcgtcgacaagccggcc

SEQ ID NO: 501 - DNA, 46 bp, synthetic construct
  source 1..46: mol_type = other DNA
ccggatgatcctgacgacggaggacgccgtcgtcgacaagccggcc

SEQ ID NO: 502 - DNA, 46 bp, synthetic construct
  source 1..46: mol_type = other DNA
ccggatgatcctgacgacggagatcgccgtcgtcgacaagccggcc

SEQ ID NO: 503 - DNA, 46 bp, synthetic construct
  source 1..46: mol_type = other DNA
ccggatgatcctgacgacggagagcgccgtcgtcgacaagccggcc

SEQ ID NO: 504 - DNA, 46 bp, synthetic construct
  source 1..46: mol_type = other DNA
ccggatgatcctgacgacggagaacgccgtcgtcgacaagccggcc

SEQ ID NO: 505 - DNA, 38 bp, synthetic construct
  source 1..38: mol_type = other DNA
atgatcctgacgacggagaccgccgtcgtcgacaagcc

SEQ ID NO: 506 - DNA, 38 bp, synthetic construct
  source 1..38: mol_type = other DNA
atgatcctgacgacggagttcgccgtcgtcgacaagcc

SEQ ID NO: 507 - DNA, 38 bp, synthetic construct
  source 1..38: mol_type = other DNA
atgatcctgacgacggagtccgccgtcgtcgacaagcc

SEQ ID NO: 508 - DNA, 38 bp, synthetic construct
  source 1..38: mol_type = other DNA
atgatcctgacgacggagtgcgccgtcgtcgacaagcc

SEQ ID NO: 509 - DNA, 38 bp, synthetic construct
  source 1..38: mol_type = other DNA
atgatcctgacgacggagtacgccgtcgtcgacaagcc

SEQ ID NO: 510 - DNA, 38 bp, synthetic construct
  source 1..38: mol_type = other DNA
atgatcctgacgacggagctcgccgtcgtcgacaagcc

SEQ ID NO: 511 - DNA, 38 bp, synthetic construct
  source 1..38: mol_type = other DNA
atgatcctgacgacggagcccgccgtcgtcgacaagcc

SEQ ID NO: 512 - DNA, 38 bp, synthetic construct
  source 1..38: mol_type = other DNA
atgatcctgacgacggagcgcgccgtcgtcgacaagcc

SEQ ID NO: 513 - DNA, 38 bp, synthetic construct
  source 1..38: mol_type = other DNA
atgatcctgacgacggagcacgccgtcgtcgacaagcc

SEQ ID NO: 514 - DNA, 38 bp, synthetic construct
  source 1..38: mol_type = other DNA
atgatcctgacgacggaggtcgccgtcgtcgacaagcc

SEQ ID NO: 515 - DNA, 38 bp, synthetic construct
  source 1..38: mol_type = other DNA
atgatcctgacgacggaggccgccgtcgtcgacaagcc

SEQ ID NO: 516 - DNA, 38 bp, synthetic construct
  source 1..38: mol_type = other DNA
atgatcctgacgacggagggcgccgtcgtcgacaagcc

SEQ ID NO: 517 - DNA, 38 bp, synthetic construct
  source 1..38: mol_type = other DNA
atgatcctgacgacggaggacgccgtcgtcgacaagcc

SEQ ID NO: 518 - DNA, 38 bp, synthetic construct
  source 1..38: mol_type = other DNA
atgatcctgacgacggagatcgccgtcgtcgacaagcc

SEQ ID NO: 519 - DNA, 38 bp, synthetic construct
  source 1..38: mol_type = other DNA
atgatcctgacgacggagagcgccgtcgtcgacaagcc

SEQ ID NO: 520 - DNA, 38 bp, synthetic construct
  source 1..38: mol_type = other DNA
atgatcctgacgacggagaacgccgtcgtcgacaagcc

SEQ ID NO: 521 - DNA, 34 bp, synthetic construct
  source 1..34: mol_type = other DNA
ataacttcgtatagcatacattatacgaacggta

SEQ ID NO: 522 - DNA, 34 bp, synthetic construct
  source 1..34: mol_type = other DNA
taccgttcgtatagcatacattatacgaagttat

SEQ ID NO: 523 - DNA, 27 bp, synthetic construct
  source 1..27: mol_type = other DNA
cacaattaacatctcaatcaaggtaaa

SEQ ID NO: 524 - DNA, 50 bp, synthetic construct
  source 1..50: mol_type = other DNA
aaaggagttttttagttaccttaattgaaataaacgaaataaaaactcgc

SEQ ID NO: 525 - DNA, 34 bp, synthetic construct
  source 1..34: mol_type = other DNA
caggtttttgacgaaagtgatccagatgatccag

SEQ ID NO: 526 - DNA, 57 bp, synthetic construct
  source 1..57: mol_type = other DNA
tggtgctgagtagtttcccatggatcactgtccagagacaacaacccagcacccgaa

SEQ ID NO: 527 - DNA, 38 bp, synthetic construct
  source 1..38: mol_type = other DNA
ggtactgtggcggttgtactgattcacttccccatatc

SEQ ID NO: 528 - DNA, 38 bp, synthetic construct
  source 1..38: mol_type = other DNA
cataaatggtacaactaatgtggcacctgtaccaccca

SEQ ID NO: 529 - DNA, 38 bp, synthetic construct
  source 1..38: mol_type = other DNA
cttatatttacaggaccaactggatctggaaaaacaac

SEQ ID NO: 530 - DNA, 50 bp, synthetic construct
  source 1..50: mol_type = other DNA
cattatatgttcttacagtatggcggcccggattgtaaaaacatataatg

SEQ ID NO: 531 - DNA, 50 bp, synthetic construct
  source 1..50: mol_type = other DNA
cgttatagggtattacagtatggcggtcggtactgcaataccctataacg

SEQ ID NO: 532 - DNA, 50 bp, synthetic construct
  source 1..50: mol_type = other DNA
tgtatcattttcatatagttagcacctgcacactatatgaaaatgataca

SEQ ID NO: 533 - DNA, 50 bp, synthetic construct
  source 1..50: mol_type = other DNA
tgtctactatctgtatatgcgacacatgtggcataaagacatagtagaca

SEQ ID NO: 534 - DNA, 50 bp, synthetic construct
  source 1..50: mol_type = other DNA
catcgaccctgacgcatgcggaggcggcgctccatgcgtctgacctcatt

SEQ ID NO: 535 - DNA, 50 bp, synthetic construct
  source 1..50: mol_type = other DNA
gttagtacccaaatgacaaaaggtcatccttttatcatttgggtactaac

SEQ ID NO: 536 - DNA, 50 bp, synthetic construct
  source 1..50: mol_type = other DNA
cttattaaaacccgttccgcttctgtcaaagcggcatcggttttataaac

SEQ ID NO: 537 - DNA, 50 bp, synthetic construct
  source 1..50: mol_type = other DNA
ggcgtgatggtcgtgaacctcaacatgacgacgaacacgacctcgcggcc

SEQ ID NO: 538 - DNA, 50 bp, synthetic construct
  source 1..50: mol_type = other DNA
tctacatcttgaatatatcaagttataactttgaattatatcagtttata

SEQ ID NO: 539 - DNA, 50 bp, synthetic construct
  source 1..50: mol_type = other DNA
aattatatctaaaagcactaagctccgccatactgcttttagatataata

SEQ ID NO: 540 - DNA, 52 bp, synthetic construct
  source 1..52: mol_type = other DNA
gtggtttctttgatgaacaactgcagtctcagtggtgtacggtacaaaccca

SEQ ID NO: 541 - DNA, 52 bp, synthetic construct
  source 1..52: mol_type = other DNA
gtggtttgtttggttaaccacagcggtctcagtggtgtacggtacaaaccca

SEQ ID NO: 542 - DNA, 52 bp, synthetic construct
  source 1..52: mol_type = other DNA
gtggtttgtttgatgaaccactgaagtctcagtggtgtacggtacaaaccca

SEQ ID NO: 543 - DNA, 60 bp, synthetic construct
  source 1..60: mol_type = other DNA
ggcggcggcggcagcggcggcggcggcagcggcggcggcggcagcggcggcggcggcagc

SEQ ID NO: 544 - DNA, 24 bp, synthetic construct
  source 1..24: mol_type = other DNA
ggcggcgcgagcccggcgggcggc

SEQ ID NO: 545 - DNA, 36 bp, synthetic construct
  source 1..36: mol_type = other DNA
ggcggcgcgagcccggcggcgccggcgccggcgggc

SEQ ID NO: 546 - DNA, 126 bp, synthetic construct
  source 1..126: mol_type = other DNA
gcggaagcggcgaaagaagcggcgaaagaagcggcgaaagaagcggcgaaagcgctggaagcggaagcggcggcgaaagaagcggcggcgaaagaagcggcggcgaaagaagcggcggcgaaagcg

SEQ ID NO: 547 - DNA, 66 bp, synthetic construct
  source 1..66: mol_type = other DNA
gcgcatcatagcgaagatccgggcggcggcggcagcggcggcggcggcagcggcggcggcggcagc

SEQ ID NO: 548 - DNA, 45 bp, synthetic construct
  source 1..45: mol_type = other DNA
ggcggcggcggcagcgaagcggcggcgaaaggcggcggcggcagc

SEQ ID NO: 549 - DNA, 45 bp, synthetic construct
  source 1..45: mol_type = other DNA
gaagcggcggcgaaaggcggcggcggcagcgaagcggcggcgaaa

SEQ ID NO: 550 - DNA, 102 bp, synthetic construct
  source 1..102: mol_type = other DNA
agcggcggcagcagcggcggcagcagcggcagcgaaaccccgggcaccagcgaaagcgcgaccccggaaagcagcggcggcagcagcggcggcagcagcacc

SEQ ID NO: 551 - protein, 20 aa, synthetic construct
GGGGSGGGGSGGGGSGGGGS

SEQ ID NO: 552 - protein, 8 aa, synthetic construct
GGASPAGG

SEQ ID NO: 553 - protein, 12 aa, synthetic construct
GGASPAAPAPAG

SEQ ID NO: 554 - protein, 42 aa, synthetic construct
AEAAKEAAKEAAKEAAKALEAEAAAKEAAAKEAAAKEAAAKA

SEQ ID NO: 555 - protein, 22 aa, synthetic construct
AHHSEDPGGGGSGGGGSGGGGS

SEQ ID NO: 556 - protein, 15 aa, synthetic construct
GGGGSEAAAKGGGGS

SEQ ID NO: 557 - protein, 15 aa, synthetic construct
EAAAKGGGGSEAAAK

SEQ ID NO: 558 - protein, 34 aa, synthetic construct
SGGSSGGSSGSETPGTSESATPESSGGSSGGSST

SEQ ID NO: 559 - DNA, 31364 bp, synthetic construct
  source 1..31364: mol_type = other DNA
cgaacttgtttattgcagcttataatggttacaaataaagcaatagcatcacaaatttcacaaataaagcatttttttcactgcattctagttgtggtttgtccaaactcatcaatgtatcttatcatgtctggatctccccgctgtggcggccgctctagaactagtggatcccccgggctgcaggaattcgatatcaagcttatcgataccgtcgacctcgagtttaaacggtactcatcagactttcctcttcttcttgggctcgaattcgctgccgtcggcggttctttttgagccgccagagggtgatgaattttctatgaggagggtagaggtgtctggagtctctgtgatggctgcctttcgggccgcttggtcagccatccggttgcctctagcctcggcgctgtgtcccttttgatgtcctggacaatggattatgctaagtcttttgggcagaaagagggcttttagtagggccaagatctcgtctttatttttgatctctttgccttctgatgtgagccacccacgccttctgtatatttctccatggatatgggcagtagcaaaagcataacggctatcagtataaacatttagcttcttaccttctgccatctttagggcctgggtgagtgctatcagttcagcccgctgagcggatgtcccggctggcagggctttagcccagattacctcggtctcggtggtcaccgcagctcccgccttacgctgtccctcttgtaagagactgcttccatccgtgtaccaggtgtggtcggcgtctgggagcggctggtccgttaggtcgggtcgggttccgtgggcttcggccaggatatcaaggcagttgtgttgcagcccttcctcaggcagtgggagcagcgtagccgggttcagggctaccaccggtccgaactggacccggtccgtgtccaaaagcaaggcctgatagtgagtcatccgggcgttggaaagccagcggtcggggggttgtttgactagtgcctctactgcatggggggccagaatgactagtggctgtcccatggttagcttgcctgcatcctttgtcagtacggcaatggctgctaccatccgtaggcaagggggccacccagctgctactgggtctagctttttggacaggtaggccaccggccgacgccaaggtcccagtttttgcgttaggacacctttggcgtagccctgcttctcgtcgacaaagagttcaaagggcttagtcaaatctggcaaccccagggctggggcagttagaagagcttgcttgatttcttgataggccttttgttggtctgggccccaattaaacagagtccccggtttggtgagagggtacaggggggctgccatttctgcaaacccagggatgaagaggcgacagaagcctgccttccctaggaactcccttagttgtcgaggggtcttcggagtaggctgccccatcacagtctcttttctggcctcagtcagccatctctgaccctcttttagaagataccccagatacttgacctgtttctggcaaatttgggctttcttggccgaggcccgatacccgaggttccctagggtttgtaacagggcccgagtaccttgttggcagtctagctcagaagtggcggccagcagtaagtcatccacgtactgtagcaggatcaagtctgggtgctggatccggaagtctgctaggtctctgtgcagtgcctcattaaacagggtgggactgtttttgaaaccctgtgggagtctggtccaggtcaattgtcctgagattcccatctctggatctctccactcaaaggcgaagagaggctgactggtggggtggagtctcaggcagaaaaaggcatcctttaaatcaagcacagtgtaccactggtgggacggtgggagcccgctcaagaggttgtaagggttgggcacggtggggtggatgtcttccacccgcttgttgacttctctcagatcctggacaggcctataatcattagtccctggtttcttaacgggtagcaggggcgtgttccagggggactggcagggtaccagtattccctggtccaacagtctctgtatgtggggcttgatccccagtctggcttcttgtgacatggggtattgttttatggacacgggggtagaggttgctttcagaggtatgatcagaggagcttggcgaactgccagtcccatgcccccggtttccgcccaggcctgaggaaaatcagacagccatgtggaccctagagaaacatctggctcttttgaggtctcatgtagccgatactcatcttctatatttagggtgctgctgccgccgctgctgccgccactgctctctggtgttgctgactcgcttgttcctggtgtctcgctgccagaggatcctccgctagatcctccagagtcacctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatagcgtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatgtccaggccgatgctgtacttcttgtcgactttccgcttcttctttggtgactcgaactcgcttccgtcggctgtccgtttcatgtaccaactgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcgaatggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcaagagaaatgttctggcacctgcacttgcactggggacagcctattttgctagtttgttttgtttcgttttgttttgatggagagcgtatgttcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattcgagctcggtacccggggatcctctagtcagctgacgcgtgatgccaaaaaagaaaagaaaagtgtatccctatgatgtccccgattatgccggttcaagagccctggtcgtgattagactgagccgagtgacagacgccaccacaagtcccgagagacagctggaatcatgccagcagctctgtgctcagcggggttgggatgtggtcggcgtggcagaggatctggacgtgagcggggccgtcgatccattcgacagaaagaggaggcccaacctggcaagatggctcgctttcgaggaacagccctttgatgtgatcgtcgcctacagagtggaccggctgacccgctcaattcgacatctccagcagctggtgcattgggctgaggaccacaagaaactggtggtcagcgcaacagaagcccacttcgatactaccacaccttttgccgctgtggtcatcgcactgatgggcactgtggcccagatggagctcgaagctatcaaggagcgaaacaggagcgcagcccatttcaatattagggccggtaaatacagaggctccctgcccccttggggatatctccctaccagggtggatggggagtggagactggtgccagaccccgtccagagagagcggattctggaagtgtaccacagagtggtcgataaccacgaaccactccatctggtggcacacgacctgaatagacgcggcgtgctctctccaaaggattattttgctcagctgcagggaagagagccacagggaagagaatggagtgctactgcactgaagagatctatgatcagtgaggctatgctgggttacgcaacactcaatggcaaaactgtccgggacgatgacggagcccctctggtgagggctgagcctattctcaccagagagcagctcgaagctctgcgggcagaactggtcaagactagtcgcgccaaacctgccgtgagcaccccaagcctgctcctgagggtgctgttctgcgccgtctgtggagagccagcatacaagtttgccggcggagggcgcaaacatccccgctatcgatgcaggagcatggggttccctaagcactgtggaaacgggacagtggccatggctgagtgggacgccttttgcgaggaacaggtgctggatctcctgggtgacgctgagcggctggaaaaagtgtgggtggcaggatctgactccgctgtggagctggcagaagtcaatgccgagctcgtggatctgacttccctcatcggatctcctgcatatagagctgggtccccacagagagaagctctggacgcacgaattgctgcactcgctgctagacaggaggaactggagggcctggaggccaggccctctggatgggagtggcgagaaaccggacagaggtttggggattggtggagggagcaggacaccgcagccaagaacacatggctgagatccatgaatgtccggctcacattcgacgtgcgcggtggcctgactcgaaccatcgattttggcgacctgcaggagtatgaacagcacctgagactggggtccgtggtcgaaagactgcacactgggatgtcctagcgcggccgcatcgataagcttgtcgacgatatctctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcctcgagcttcctcgctcactgactcgctgcgctcggtcgttcggctgcggcgagcggtatcagctcactcaaaggcggtaatacggttatccacagaatcaggggataacgcaggaaagaagttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactaagtaaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaaccggcctcttcatcgggaatgcgcgcgaccttcagcatcgccggcatgtccccctggcggacgggaagtattgcattcggaccgcactttttttaaataaaaaaaatttttttctctctcttttttttttttagagacgaagtctcgctatgttgcccaggctggtcttgactagagatactccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgaattcaagctgctagaggatcttgctatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaataaccgggcagggggatcgatccgaattcccgggctcgagtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatcatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgaatctatgtcgggtgcggagaaagaggtaatgaaatggcagagcaagatttgtggtttgtctggtcaaccaccgcggtctcagtggtgtacggtacaaacccaatgggcaacagcttcagcaccagcgccttcggccctgtggccttttctctgggcctcctgctcgtgctgcctgccgcttttccagctcctgtgttcaccctggaagatttcgtgggagattggcggcagaccgccggctacaacctggaccaagtgctggaacagggcggagtgtccagcctgtttcagaacctgggcgtctccgtgacccctatccagcggatcgtgctgagcggcgagaacggcctgaaaatcgacatccatgtgattatcccctacgagggcctgagcggagatcagatgggccagatcgagaaaatcttcaaggtggtgtaccccgtcgacgaccaccacttcaaggtgatcctgcactacggcaccctggtgatcgacggcgttacccctaacatgatcgactacttcggcagaccctatgagggaattgccgtgttcgacggcaagaaaatcaccgtgaccggcacactgtggaacggcaacaagatcatcgatgagcgcctgatcaacccagacggcagcctgctgttcagagtgacaatcaatggcgtgacaggctggagactttgtgaaagaatcctggccgccaccaacttcagcctgctgaagcaggccggcgacgtggaagagaatcccggacctggttctggcgagggcagaggatctctgctgacatgcggcgatgtggaagagaatcctggacccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagaagcagagccaggtgagtttggacaaaccacaactagaatctgtgccttctagttgccagccatctgttgtttgcccctcccccgtgccttccttgaccctggaaggtgccactcccactgtcctttcctaataaaatgagaaaattgcatcgcattgtctgagtaggtgtcattctattctggggggtggggtggggcaggacagcaagggggaggattgggaagacaatagcaggcatgctggggatgcggtgggctctatggaaatactccatctctcgcgaaaaaaaaaacgcgtcaaacaaaaagcgctataaaagataagccctcacgtgttattttttctgtcttactctttgaatcatctaaatgtatcttaatgtcaggagtgaggtagaggacccagttgtttggtttaaatgactagccagttttcccaacaccaggtgttgaacatccctcactgatgtgagatgctgcctttttcacagtctcaatccccatgtgcatttctatttattttccttccactttattcacatctttttcagtatccacaaccatattgttttaaatagcttcatgatatgttttaatatttggtagagctagaaccctttcttggttctttttcttttcttcttctcctccttttcctcctccttctttctcctcctccttcttctttctttttagaaatttcccggctattcttgttttgtagtcttccagataaataattttgaatcactttgtcatgttccagaaaataatactgccagaatttttattgtgatcacagaagttttacagattcatctgggggaaaaaaacccatcttcccagaatacagaaagggtgagggaaagacaaacatcaaggacaagtctcaggttctggacttggagaccaagaggatttggggcatccgggagcagggcagggaggtgtgatggctgcctttcactgagtaggaggatgcccagacttgggggaactatggaaagtttggttcagactttctgagttggagatgtctatgcgcagtccacagggcgaagccccggaggtagaggccctccttactgcctccctcattcagtctgaggtccattctccacatggacactgaagtgactcaaggcttcccttgttggatcaggatagtctccagggccccgggtggtctgggcctgcccacctccccatcccctctcctgcctgccctcctcttgctcactggaccccacccaccagcctccttctattcctcctgacatttgcacttgctgttccgtctctgcttggaacatcctagctctttgcatggcttgctccttcccatcttttaatctaagctcaaatatcactccttcagatatgcctttcccaaatatcctagctaaagagaactgctccccacttcccacaagccctagcaaactgggactctccttcttgctttctttctctctgccccttctgtgttttttccatagaccttaacttgttactgttttatttgttgatgtgtttacatcatttgtccccctgtgatggttagttttatgtgtcaacttggctaggctgtaggggctgttcgtcaatcaaacactgatctatgtgtagctgtgaagggattttgtagctgtgattaatagttgactttaagtaaggaagattatcctggagaatgtgggggaaggggcagacctcagccaatcagttgaaaggccttaagaacaaagctgagatttccccgaggaagaagaaattctgcctgtggactgcagcatcagctcctgcttgagaggttccaggctgcccttcctgacagcctgtcctatgattttggacttgccttgccaggtttttcctctttcttgaccccccaaaaccatatatatctcctgctggtttgtttctctggtgtaatcctgagggatgaacactcctccctcccagttcactctgggagaggttggggatcatgtttgtctatttccccatgcccggcccatgcgtgtttattgaatcactgaacaaccacgcattaggaagccagtcatatgtgcttcttcagaacctcatgtccattgccagatctccccggcttttgtggctagaggacaagtgagagatagtagcttacccacagaccttggccctgaggcccccaggggcctgagcctgctgtaagggaggagggagcccctgaggtctcacacacctccctggggatctggcattttcccctggggctggcctcagagctgggcgggggcagattatggagtgggttgtagagggagcacctccccactagcaatcatggttttttctgcgcctctcatagggaggcctacaggcctccaccttcagctcttcaggagccacaggcagctgagtgacttctgcatcacagtcttctcagagacaaacttgcagtaagaaagaggggcccgtgagggaccccagagaaggctgttgtcaaagcaggatgagagtgaactcttccatgggggacacccagcgtctccaagctcttttatgctgtcttcaaggggtctagagagcttcagcccaacatgagacccagtccagcagcatttccctgcgaagtgaaagttaggaccctgactagatacaccacgctgacctcagccagaatatcaagatgctgaggcgctgagatgctgggatgctaaggtgctaaggtgctggggtgctggggtgctaggatgctgaggttttgtgatactgggtcgctgagatgctgggatactggggtgctgaagtgctggggtactggggtgttgccatgctgaaatgctggggtgttgggatgctgaggtgctgggttgctgggatgctgggatagtccttggatgctgcggtgctgagatgctggtctgctggtctgctggggtgttgggatgctgggattttgggatgctggagtgctgcagtgctgcagtgctgagatgctggagtgctggggtgctggaatactgtagtactggtgtgctggagcattgagatgctagggcactgggatgctaaggtgctgagatgctgcagtgctggggtgttgggatgccgaggtgttgggatgcttaagtgctggggtgttgggatgctggggtgctggaacactatggtgctggggtgctggagtgttgagatactgtagtggtgggatgcttaagtgctggggtgctgggtgttgggatgcctaggtgctggggtgccgagatgctggagtactagtgtgctgggatgctgaagtgctggggtcctgagatgctgcagtgctagggtgctgagattctgggctgctggagtgttggggtgctgggatgctggagtgctgagatgcttggacaatggggtgctggaatactatggtgctggggtgctggggtattgagatgctagggtactgggatgctgaagtgctgagatcccggagtgctgggctgctgggccacaggctcttgaatccattcgtctgcccaggggaagaaaccagaagataaagagctaatgaaggagctttggttgagagggaggaagtaatggaaggagcaacatcttgtggaggagcaggagagaatggacctcaggttgggagagagggccgggctacaggccagagaggcagaaggattccagcagagtgtgggctccgggagccaaggggaaacaggtttctgggaggagagagtccagtactgctgaagtggcaagtccgctgaggaccgggaagcttcatttggctttatgaccgggaggaatttggaactgtgactagagtacttagggggaaggaggcaagactggagccagattgctctgggttgaggggtgagtgggaggtgaagcagggcactgtcactcctttgaagggtggcagagagctggaattggtgctggatgggctgtggggtgacagggtcatgtggaaagcccctggggggcactcggaaaaggagaagctgacagtacagtgagaggacagctaagggaaagcggaatggcagaacacgcactgccgggaggaatgaggatagggtcaggagtgccgggggcagtgaggccagcctggggtcaggtggcaggacgtgtccgggaagctggtctgcactgcagcccacactggctcagccttgaggttccctgtgtggttggggtaggaagttgaaccctctgggaatggaagatggaaccagctctgcgagccaagctcagcttttatctatgggtctctgagggctggcagagctgagtggggacaactgtgatccgtgaggctctcaggttgaggtggcccctccgggagggcttcattttcccagcgggtaggttctaagcagcagtggctgggcaggtgggtccaacacagagccagagaagggtgaatgggcctcccggcaccccacccctgctgcccctgagctcagtgatggagggggacagcacagctgagcccaagtgctttggtgtggccctgagggaaagctgcagcctgcctggggcccggcatggatgggacacttgaggcagagggacaatagtgggcgctgcagtgaggctggctcttggagaggtttcctgaggagtgctgcctgagacgggcagggagaacagagacaaagttggtgacagggaatgaaagctgactgaaggactttacccagacctatgaggatatctctctcagcaggaagcaggaggggactgtgtgaggactggccaagagctggagtgttgggaaaatgactctttctccgacccctctgtcctagctctggcccccggactgcggaggtctgcttccacccccattggtcgatcgttgtcccttgtcacagccattgagaattttggcagggagcatgttcttagagcatttttaggctctgcgggacataacagctctgcctcagagcacatgcctttctcagctcctgaaagccactgatcaaattggaacattttgtaccttagggatgaggatatcaactctcccagccacttagagggataaatgtgatgatgcattcaattgtgactacatctcccaactgttgcttcagctgctctcctatagcacatggcgggaggcgtgcatcccagtagctacctccccacttttggggagatgtggttccatccatgaaacccgggtacccgcctaccaggtcccggcctatcaggtggcagggtctggtcaaagaagggcatgtgtggtcttcagcaagggagacaggacggtggtgcagagcgtctagaccctcagggcaagtctcccccacacctgctcccggggcagttgtctttgtgacctcccatccccctctgtttcatcctctataaaatgaggggctgagccccaaaataacaggcttctttgccatgatgcaaaactgctgaatctttctttctgacacacaaggcatcgagcagcctctgaaagaaccaaagccactagcaggcttcctgacttgggtttgtaggtactgaatactcccttgaaaaataaaaacatagaggcacttttctcctggctgtttattacagaacgaagaaaaaacacactggcttgaaacagacgccagatttcaaatgtagaggtgaaatacgaggtggcaattaaaatgtgattacagaaagtctggacactgagaaaagtttacaggacagtgggtgtgggttttctataacagacacttaaatatacatgacgataattgcagatagaaaccatcaaagacaaaccccaaatcaactaataatgtttacagatgttcccccccaaaccacagagccttacatcaaaacaaatactgaaaggctttaaaccaggaacagctcgccttaaccccacgagggtgcacacaagctgggctttttctctcggtctgaatggtaaagggaggaggatactctagctcctccaggtggattgctgagacagggctcggctcacacactgtctctgcgcctctcccaaatctggagaactctcccagcctcctggtaaagtgtctctgtggggcacttaacgataaaacagcttctgctgtaaagctcattaggaaagagctagcggagactgaaaggttcgcaaaagagattaagaatcacacaaggcaataggatttttagtgaacatagaaataaatggccaagtggttttctatttggcatttgtcaacttgcacaacaactcttggtcatatccacattgctcattgcattaaaaccataagcgactcagccacctagcttaacaaggtatcactggagcaaacaacacggtctgcatatttgtaacattgtataataaacacaaaacaatgcatagtaaacacaactctactgaaacaaaagccgtcgctttatttacaaagtcacaaaatgaagtataaatacttctgtcattaatgtttaggaaaaccatttacaaaattttcaaatatgtacacgtagcttgaaaaatcaccagctttccattttgtcacaggtagagagagggataagcatgggctgacaacaccactcaaattgtaacgggagacaactgcgggtatggatcgacaccacttcctagagtgatgtcaccatgggggtttctatgggcatcctgctcagatttaaagtgccccagcatcctgggtgacttgcccagaattctgggctgtggcattttgagcagcagcatgctgttccaaaatgtcgtcgatcagcctcaagttgcacacccagtcttcatctgggctcacacaggagcctttcaagagagcttcaatgaaatctacctcattgcagtcaggtgacgaaatcagatcatttagtgggggttggggctggcgcaaaaagtcggcaggtggcagctcagggggaatatccgttctgtcgaacggacctgggaactggctggcagcaacggcagaagcagcagcagcggtggcagcagcagccacatagcttggtggctcgatgccctgtatggggctcaggggactaaagctggccataccctgctggaggaacttggtggtgtttgctacaggcaccgggccctgtaccgggctctgcctgaggctctggctgcccagcaggctgaagctggggttgttggccgggggcacttgtgttcccatcgcagcgggcacttgtgcctcccaatcagatggcctctgaaggcaggcctggccagaaggtgagtgctgctgaacgctattatccacttggctgaggggtgttttccccgaaactgctgtggtcacagctgctgccgctgtgacccatgcagcattgttgaacgcagtgggcattcttggcacactaggccgtctgagctggtggggactcaaggactgggtgcccagggagctgggacagaacccaggcaggggcacttctggtggggtggccttggggctctgcatatgctggcagacagagtcaagtctgcccgggggagtctggcctgagtgtgagaggatgggacactgggggctggaggtgaaaattccttgccgcttccccagagttggtgagatcactcccatgccctcgcagctctggtgcctggtgagtgggatcattcccggactcagattgttctgaagaagcccagttctgggtggcatcaagtgcttgctagatggggggcttgccttgatccggctacacttggaggtgacttgttcttggacggctacatacagaaagagagaagtggggatgagttccaaaggcatcctcgacttcggctgtggccaccggagggtagctcctggcccaacacggacttctcacctcccgcccttggctctctactgagctcccccctgctccccaattcctcgccattcccctcatttctctgccctcagcccggactgcagttcttctgggaagctgccccaactccctaggtctgtgctcaccaagagcagatcacactggactgaaatgccagctgatttgtctcttcaagaaaattggaagctcctggaggtcagggtccatgtctgcttttacactcagtgctctgtatgcaggcccggcactgcccaccctttgacaggtggtgcatattttgtagaaggaaggaaggggccgggtggggtgggctgggctggtggcgggagctagctcagcctcttagattctctacccgatggatgtgacctgggacagcaagtgagtgtggtgagtgagtgcagacggtgctttgttcccctcttgtctcatagcctagatggcctctgagcccagatctggggctcagacaacatttgttcaactgaacggtaatgggtttcctttctgaaggctgaaatctgggagctgacattctggactccctgagttctgaagagcccggggatggagagacacggagcagaagatggaaggtagagtcccaggtgcctaagatggggaatacatctcccctcattgtcatgagagtccactctagctgatatctactgtggccaatatctaccggtacttttttggggtggacactgagtcatgcagcagtcttatggtttacccaaggtcaggtaggggagacagtgcagtcagagcacaagcccagtgtgtctgacccacccaagaatccatgctcgtatctacaaaaatgattttttctcttgtaatggtgcctaggttcttttattatcatggcatgtgtatgtttttcaactaggttacaatctggccttataaggttaacctcctggaggccaccagccttcctgaaacttgtctgtgctgtccctgcaactggagtgtgcctgatgtggcactccagcccggacaagtgggacacagactccgctgttatcaggcccaaagatgtcttccataagaccagaagagcaatggtgtagaggtgtcatgggctacaataaagatgctgacctcctgtctgagggcaagcagcctcttctggccctcagacaaatgctgagtgttcccaagactaccctcggcctggtccaatctcatcccactggtgcgtaagggttgctgaactcatgacttcttggctagcctgcaacctccacggagtgggaactacatcaggcattttgctaactgctgtatcctaggccaataaatgttgatcacatttatagctgccatggtagggtggggacccctgctatctatctgtggaggctctgggagcccctgacacaaactttctgaagcagagcctccccaaccccttttccattccctatacctgacagatggcccaggaacccattagaaatggaaggtcactgcagcagtatgtgaatgtgcgtgtgggagaagggcaggatcagagccctgggggtgtggcagcccccaagtgattctaatccagatcctagggttgtttccctgtcccattgaaatagctgctttaaggggcctgactcagggaaatcagtctcttgaattaagtggtgattttggagtcatttagaccaggccttcaattgggatccactagttctagagcggccgggcccagggaaccccgcaggcgggggcggccagtttcccgggttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccactacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggttacctgttgtttctcccactaaaactttttaagggcaggaatcaccgccgtaactctagcacttagcacagtacttggcttgtaagaggtcctcgatgatggtttgttgaatgaatacattaaataattaaccacttgaaccctaagaaagaagcgattctatttcatattaggcattgtaatgacttaaggtaaagagcagtgctattaacggagtctaactgggaatccagcttgtttgggctatttactagttgtgtggctgtgggcaacttacttcacctctctgggcttaagtcattttatgtatatctgaggtgctggctacctcttggagttattgagaggattataagacagtctatgtgaatcagcaacccttgcatggcccctggcggggaacagtaataatagccatcatcatgtttacttacatagtcctaattagtcttcaaaacagccctgtagcaatggtatgattattaccattttacagatgaggaacctttgaagcctcagagaggctaacagacataccctaggtcatacagttattaagagaaggagctctgtctcgaacctagctctctctctctcgagtaataccagttaaaaaataggctacaaataggtactcaaaaaaatggtagtggctgttgtttttattcagttgctgaggaaaaaatgtgggcaaatgagctattaactataacggtcctaaggtagcgagtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatcctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgtttaaactacggcccggtagggataacagggtaattacccagcttttgtctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccggagcttagggataacagggtaatccactacgtagtttaaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaacttcgaaaaaaggccacctccgagatgatcctgacgacggagaccgccgtcgtgcaccgactcggtgccactttttcaagttgataacggactagccttatttaaacttgctatgctgtttccagcatagctcttaaacactctcggaggtggccagtacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctctcaagcaggagctgatgctgcagtccacagaaaaaacgttttacccggaggcttgtcgacgacggcggtctccgtcgtgcaccgactcggtgccactttttcaagttgataacggactagccttatttaaacttgctatgctgtttccagcatagctcttaaactgctccgggtaaaacgcgtcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcgagatttccccgaggaagaagaaattctgc